SEQUENCE LISTING (5 sequences)

SEQ ID NO: 1 - protein, 17 aa, synthetic construct
  REGION 1..17: note = Synthetic
NVTNNFDERPISLGGNK

SEQ ID NO: 2 - protein, 8 aa, synthetic construct
  REGION 1..8: note = Synthetic
VSDFGLAR

SEQ ID NO: 3 - protein, 13 aa, synthetic construct
  REGION 1..13: note = Synthetic
SANILLDEDFTAK

SEQ ID NO: 4 - protein, 10 aa, synthetic construct
  REGION 1..10: note = Synthetic
FHSFSFYELK

SEQ ID NO: 5 - protein, 14 aa, synthetic construct
  REGION 1..14: note = Synthetic
IVGTTAYMAPEALR

(19) United States
(12) Patent Application Publication
(10) Pub. No.: US 2024/0343733 A1
(43) Pub. Date: Oct. 17, 2024

(54) COMPOUNDS FOR TARGETING DEGRADATION OF IRAK4 PROTEINS

(71) Applicants: BIOGEN MA INC., Cambridge, MA (US); C4 THRAPEUTICS, INC., Watertown, MA (US)

(72) Inventors: Kevin M. Guckian, Northborough, MA (US); Emily Anne Peterson, Belmont, MA (US); Fang Gao, Somerville, MA (US); Ryan Evans, Somerville, MA (US); Eric Stefan, Watertown, MA (US); Jeremy L. Yap, Sudbury, MA (US); Corey Don Anderson, Brighton, MA (US); Morgan Welzel O'Shea, Waltham, MA (US); Jae Young M. Ahn, Somerville, MA (US); Christopher G. Nasveschuk, Stoneham, MA (US); James A. Henderson, Weston, MA (US)

(21) Appl. No.: 18/576,940

(22) PCT Filed: Jul. 7, 2022
(86) PCT No.: PCT/US2022/073522

§ 371 Date: Jan. 5, 2024

Related U.S. Application Data
(60) Provisional application No. 63/219,160, filed on Jul. 7, 2021.
(60) Provisional application No. 63/354,017, filed on Jun. 21, 2022.

(51) Int. Cl. C07D 487/04; A61K 31/4545; A61K 31/513; A61K 31/519; C07D 401/14; C07D 471/04; C07D 487/10; C07D 519/00

(52) U.S. Cl. CPC C07D 487/04; A61K 31/4545; A61K 31/513; A61K 31/519; C07D 401/14; C07D 471/04; C07D 487/10; C07D 519/00

(57) ABSTRACT

This disclosure relates to compounds of Formula (A): IRAK-L-DSM (A), or a pharmaceutically acceptable salt thereof, wherein DSM is a degradation signaling moiety that is covalently attached to the linker L, L is a linker that covalently attaches IRAK to DSM; and IRAK is an IRAK4 binding moiety represented by Formula (I) that is covalently attached to linker L; in which all of the variables are as defined in the application. Compounds or pharmaceutically acceptable salts thereof as described herein are capable of activating the selective ubiqitination of IRAK4 proteins via the ubiquitin-proteasome pathways (UPP) and cause degradation of IRAK4 proteins. The present disclosure also provides methods of treating disorders responsive to modulation of IRAK4 activity and/or degradation of IRAK4 with at least one compound described herein.

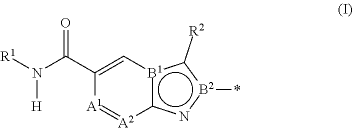

### CROSS-REFERENCE TO RELATED APPLICATIONS

This application claims priority to U.S. Provisional Patent Application Nos. 63/219,160, filed Jul. 7, 2021 and 63/354,017, filed Jun. 21, 2022. The entire contents of each of the foregoing applications are expressly incorporated herein by reference.

### TECHNICAL FIELD

Provided are certain agents that target the degradation of interleukin-1 receptor-associated kinase 4 (IRAK4), and methods of making and using such agents.

### BACKGROUND

Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection.

Covalent attachment of multiple ubiquitin molecules by an E3 ubiquitin ligase to a terminal lysine residue marks the protein for proteasome degradation, where the protein is digested into small peptides and eventually into its constituent amino acids that serve as building blocks for new proteins. There are over 600 E3 ubiquitin ligases which facilitate the ubiquitination of different proteins in vivo, which can be divided into four families: HECT-domain E3s, U-box E3s, monomeric RING E3s and multi-subunit E3s.

It is known that the ubiquitin-proteasome pathway (UPP) can be harnessed for therapeutic intervention by using chimeric compounds capable of activating the ubiquitination of a Target Protein, where the chimeric compound comprises a Target Protein binding element that is covalently linked to ubiquitination recognition element. Such chimeric compounds that are capable of binding a Target Protein and a ubiquitin ligase may cause the Target Protein to be selectively degraded via the UPP. The discovery, for example, that thalidomide binds to the cereblon E3 ubiquitin ligase has led to research investigating the incorporation of thalidomide and certain derivatives into chimeric compounds for the targeted destruction of proteins.

Protein kinases are a large multigene family consisting of more than 500 proteins which play a critical role in the development and treatment of a number of human diseases in oncology, neurology and immunology. Kinases catalyze the phosphorylation of proteins, lipids, sugars, nucleosides and other cellular metabolites and play key roles in all aspects of eukaryotic cell physiology. Especially, protein kinases and lipid kinases participate in the signaling events which control the activation, growth, differentiation and survival of cells in response to extracellular mediators or stimuli such as growth factors, cytokines or chemokines. In general, protein kinases are classified in two groups, those that preferentially phosphorylate tyrosine residues and those that preferentially phosphorylate serine and/or threonine residues.

Kinases are important therapeutic targets for the development of anti-inflammatory drugs (Cohen, 2009. Current Opinion in Cell Biology 21, 1-8), for example kinases that are involved in the orchestration of adaptive and innate immune responses. Many diseases are associated with abnormal cellular responses triggered by kinase-mediated events. Kinase targets of particular interest are members of the IRAK family.

The interleukin-1 receptor-associated kinases (IRAKs) are critically involved in the regulation of intracellular signaling networks controlling inflammation (Ringwood and Li, 2008. Cytokine 42, 1-7). IRAKs are expressed in many cell types and can mediate signals from various cell receptors including toll-like receptors (TLRs).

IRAK1 was first identified through biochemical purification of the IL-1 dependent kinase activity that co-immunoprecipitates with the IL-1 type 1 receptor (Cao et al., 1996. Science 271(5252): 1128-31). IRAK2 was identified by the search of the human expressed sequence tag (EST) database for sequences homologous to IRAK1 (Muzio et al., 1997. Science 278(5343): 1612-5). IRAK3 (also called IRAKM) was identified using a murine EST sequence encoding a polypeptide with significant homology to IRAK1 to screen a human phytohemagglutinin-activated peripheral blood leukocyte (PBL) cDNA library (Wesche et al., 1999. J. Biol. Chem. 274(27): 19403-10). IRAK4 was identified by database searching for IRAK-like sequences and PCR of a universal cDNA library (Li et al., 2002. Proc. Natl. Acad. Sci. USA 99(8):5567-5572).

IRAK4 is thought to be the initial protein kinase activated downstream of the interleukin-1 (IL-1) receptor and all toll-like-receptors (TLRs) except TLR3, and initiates signaling in the innate immune system via the rapid activation of IRAK1 and slower activation of IRAK2.

Given that IRAK4 plays an important role in signaling networks controlling inflammation, there is a great need to develop chimeric compounds capable of activating the ubiquitination and degradation of IRAK4 proteins. It is an object of the present disclosure to provide new compounds, methods, compositions and methods of manufacture that are useful for the selective degradation of IRAK4 protein in vivo via the ubiquitin-proteasome pathway (UPP).

### SUMMARY

In a first aspect, the present disclosure is a compound of formula (A):

IRAK-L-DSM (A),

or a pharmaceutically acceptable salt thereof, wherein:
DSM is a degradation signaling moiety that is covalently attached to the linker L,
L is a linker that covalently attaches IRAK to DSM; and
IRAK is an IRAK4 binding moiety represented by Formula (I) that is covalently attached to linker L;

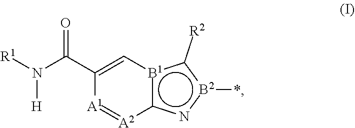

wherein:
A¹ is selected from N, CH and CR³, and A² is selected from N, CH and CR⁴, provided only one of A¹ or A² may be N;
one of B¹ and B² is N, and the other is C;
R¹ is selected from:
   i. phenyl optionally substituted with 1 to 3 R⁵,
   ii. a 5 or 6 membered heteroaryl having 1 to 3 heteroatoms independently selected from nitrogen, oxygen and sulfur, said heteroaryl is optionally substituted with 1 to 3 R⁵,
   iii. a 5 or 6 membered partially or fully saturated heterocycle having 1 to 2 heteroatoms independently selected from oxygen and nitrogen, said heterocycle may be optionally substituted with 1 to 3 R⁵,
   iv. a partially or fully saturated C₃₋₆ cycloalkyl which may be optionally substituted with 1 to 3 R⁵,
   v. a 7 to 10 membered fused heterobicyclic ring system having 1, 2 or 3 heteroatoms independently selected from nitrogen and oxygen, said heterobicylic ring system is optionally substituted with 1 to 3 R⁵, and
   vi. a 7 to 10 membered fused carbobicyclic ring system, said carbobicyclic ring system is optionally substituted with 1 to 3 R⁵;
R² is hydrogen, C₁₋₄ alkyl or halogen;
R³ and R⁴ are each independently selected from halogen, C₁₋₄alkyl, nitrile and —OR⁶, wherein the C₁₋₄alkyl is optionally substituted with C₁₋₄alkoxy or at least one halogen;
R⁵ for each occurrence, is independently selected from CN, hydroxyl, C₁₋₄ alkyl, oxo, halogen, —NR⁸R⁹, C₁₋₄ alkoxy, —O—C₁₋₄ alkyl, C₃₋₆cycloalkyl, —C₁₋₄alkyl-C₃₋₆cycloalkyl, C(O)NR¹⁰R¹¹, a C₄₋₇ heterocycle, and a 5 or 6 membered heteroaryl having 1 to 2 heteroatoms independently selected from nitrogen, oxygen and sulfur, said C₁₋₄ alkyl is optionally substituted with one or more (e.g., 1 to 6, 1 to 3, or 1, 2, 3, 4, 5 or 6) substituents independently selected from CN, halo, C₁₋₄alkoxy, and hydroxyl, said C₃₋₆cycloalkyl and heteroaryl is optionally substituted with 1 to 2 substituents independently selected from the group consisting of C₁₋₄ alkyl, hydroxyl and halogen; or two R⁵ groups together with the intervening atoms can form a ring selected from phenyl, C₄₋₆ carbocycle, C₄₋₆ heterocycle, or a 7-membered bridged ring system optionally having 1 heteroatom selected from nitrogen and oxygen, wherein said phenyl, C₄₋₆ carbocycle and C₄₋₆ heterocycle are each optionally substituted with 1 to 2 C₁₋₄ alkyl, halogen or C₁₋₄ haloalkyl;
R⁶ is hydrogen, C₁₋₅alkyl, C₃₋₆cycloalkyl, a 4 to 7 membered partially or fully saturated heterocycle containing 1 or 2 heteroatoms selected from nitrogen and oxygen, a 5 to 10 membered spiro carbocyclic ring and a 4 to 10 membered heterocycle having 1 to 2 heteroatoms independently selected from nitrogen and oxygen; wherein the C₁₋₅alkyl represented by R⁶ is optionally substituted with 1 to 3 substituents R^{6a} independently selected from halogen, hydroxyl, C₁₋₅alkyl, C₁₋₄alkoxy, C₁₋₄ haloalkoxy, C₃₋₆cycloalkyl, phenyl, a 4 to 7 membered partially or fully saturated heterocycle containing 1 or 2 heteroatoms selected from nitrogen and oxygen, and a fully saturated 5 to 8 membered bridged-heterocyclic ring system having 1 to 2 heteroatoms independently selected from nitrogen and oxygen; wherein the C₃₋₆cycloalkyl represented by R⁶ is optionally substituted with 1 to 3 substituents R^{6b} independently selected from halogen, C₁₋₄alky, C₁₋₄ haloalkyl, and C₁₋₄alkoxy; wherein the 4 to 7 membered partially or fully saturated heterocycle, the 5 to 10 membered spiro carbocyclic ring and 5 to 10 membered spiro heterobicyclic ring system represented by R⁶ is optionally substituted with 1 to 3 substituents R^{6c} independently selected from C₁₋₄alkyl and oxo; and wherein said C₃₋₆cycloalkyl, phenyl, 4 to 7 membered partially or fully saturated heterocycle represented by R^{6a} are optionally substituted with 1 to 3 R⁷;
each R⁷ is independently selected from oxo, halogen, C₁₋₄ haloalkyl and C₁₋₄ alkyl;
R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl;
R¹⁰ and R¹¹ are each independently selected from hydrogen and C₁₋₄ alkyl; and
[symbol] represents a bond to the linker L.

In another aspect, the present disclosure provides methods of treating a disorder responsive to modulation of IRAK4 activity and/or degradation of IRAK4 in a subject comprising administering to the subject an effective amount of at least one compound described herein. The present disclosure also includes the use of at least one compound described herein, or a pharmaceutically acceptable salt thereof, for the manufacture of a medicament for the treatment of a disorder responsive to modulation of IRAK4 activity and/or degradation of IRAK4. Also provided are compounds described herein, or pharmaceutically acceptable salts thereof, for use in treating a disorder responsive to modulation of IRAK4 activity and/or degradation of IRAK4. Methods of making the compounds described herein and any synthetic intermediates are also included in the present disclosure.

Other features or advantages will be apparent from the following detailed description of several embodiments, and also from the appended claims.

### BRIEF DESCRIPTION OF THE DRAWINGS

FIG. 1 (FIG. 1) shows the pharmacokinetic profile of Compound 48 following 5 mg/kg IV and 10 mg/kg PO dosing in male Beagle dogs.

FIG. 2 (FIG. 2) shows the pharmacokinetic profile of Compound 169 following 5 mg/kg IV and 10 mg/kg PO dosing in male Beagle dogs

FIG. 3 (FIG. 3) shows IRAK4 degradation following 10 mg/kg PO administration of vehicle, Compound 48, and Compound 169 in male beagle dog PBMCs.

FIG. 4 (FIG. 4) shows the pharmacokinetic profile of Compound 48 following 5 mg/kg IV and 10 mg/kg PO dosing in male cynomolgus monkeys.

FIG. 5 (FIG. 5) shows the pharmacokinetic profile of Compound 169 following 5 mg/kg IV and 10 mg/kg PO dosing in male cynomolgus monkeys.

FIG. 6 (FIG. 6) shows IRAK4 degradation following 10 mg/kg PO dosing of vehicle, Compound 48, and Compound 169 in male cynomolgus monkey PBMCs.

### DETAILED DESCRIPTION

Compounds or pharmaceutically acceptable salts thereof as described herein are capable of activating the selective ubiqitination of IRAK4 proteins via the ubiquitin-proteasome pathways (UPP) and cause degradation of IRAK4 proteins. In some embodiments, compounds or pharmaceutically acceptable salts thereof as described herein can modulate IRAK4 activities.

Compounds of the present disclosure, and pharmaceutical formulations thereof, may be useful in the treatment or prevention of conditions and/or disorders through mediation of IRAK4 function such as, for example, autoimmune disease, an inflammatory disease, bone diseases, metabolic diseases, neurological and neurodegenerative diseases, Alzheimer's disease, Ischemic stroke, Cerebral Ischemia, hypoxia, TBI (Traumatic Brain Injury), CTE (Chronic Traumatic Encephalopathy), epilepsy, Parkinson's disease (PD), Multiple Sclerosis (MS) and Amyotrophic Lateral Sclerosis (ALS).

### I. Definitions

Compounds are described using standard nomenclature. Unless defined otherwise, all technical and scientific terms used herein have the same meaning as is commonly understood by one of ordinary skill in the relevant art.

The terms “a” and “an” do not denote a limitation of quantity, but rather denote the presence of at least one of the referenced item. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the disclosure and does not pose a limitation on the scope of the disclosure unless otherwise claimed.

As used herein, the term “alkyl” refers to a fully saturated branched or unbranched hydrocarbon moiety. In some embodiments, the alkyl comprises 1 to 20 carbon atoms, 1 to 10 carbon atoms, 1 to 8 carbon atoms, 1 to 6 carbon atoms, or 1 to 4 carbon atoms. In some embodiments, an alkyl comprises from 6 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, or n-hexyl. Similarly, the alkyl portion (i.e., alkyl moiety) of an alkoxy or a haloalkyl have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons.

As used herein, the term “aryl” refers to a carbocyclic (all carbon) aromatic monocyclic or bicyclic ring system containing 6-10 carbon atoms. Examples of 6-10 membered aryl groups include phenyl and naphthyl. In some embodiments, the aryl is phenyl.

The term “bridged ring system”, as used herein, is a ring system where two non-adjacent atoms of the ring are connected (bridged) by one or more (preferably from one to three) atoms selected from C, N, O, and S. In one embodiment, a bridged ring system have from 6 to 8 ring members.

The term “fused ring system”, as used herein, is a ring system that has two ring structures sharing two adjacent ring atoms. In one embodiment, a fused ring system have from 8 to 12 ring members.

The term “spiro ring system,” as used herein, is a ring system that has two ring structures having one ring atom in common. In one embodiment, spiro ring systems have from 5 to 8 ring members.

The term “cycloalkyl” refers to partially or fully saturated monocyclic or bicyclic or spiro hydrocarbon groups of 3-7 carbon atoms, 3-6 carbon atoms, or 5-7 carbon atoms. In some embodiments, cycloalkyl is a 3- to 6-membered fully saturated monocyclic cycloalkyl (e.g., cyclopropyl, cyclobutyl, cyclopentyl, and cyclohexyl).

As used herein, the terms “carbocycle” and “carbocyclic ring” refer to saturated or partially unsaturated (i.e., non-aromatic) monocyclic or bicyclic hydrocarbon groups of, for example, 3-10, 3-8, 3-7, 3-5, 3-6, 4-6, 5-7 or 7-10 carbon atoms. 3 to 7 membered monocyclic carbocycles include, but are not limited to, cyclopropyl, cyclobutyl, cyclopentyl, cyclohexyl, cycloheptyl, cyclopropenyl, cyclobutenyl, cyclopenentyl, cyclohexenyl, cycloheptenyl, cyclobutadienyl, cyclopentadienyl, cyclohexadienyl, cycloheptadienyl and cycloheptatrienyl. Bicyclic carbocycles include, but are not limited to, bicyclo[2.1.1]hexyl, bicyclo[2.2.1]heptyl, bicyclo[2.2.1]heptenyl, 6,6-dimethylbicyclo-[3.1.1]heptyl, 2,6,6-trimethylbicyclo[3.1.1]heptyl, spiro[2.2]pentanyl and spiro[3.3]heptanyl. 7 to 10 membered bicyclic carbocycles include, but are not limited to, bicyclo[2.2.1]heptyl, bicyclo[2.2.1]heptenyl, 6,6-dimethylbicyclo[3.1.1]heptyl,2,6,6-trimethylbicyclo[3.1.1]heptyl, spiro[3.3]heptanyl, spiro[2.5]octanyl, bicyclo[3.3.0]octanyl, bicyclo[2.2.2]octanyl, bicyclo[3.3.1]nonanyl, bicyclo[3.3.2]decanyl and decalinyl.

As used herein the term “bridged-carbocyclic ring” refers to a cyclic moiety connected at two non-adjacent ring atoms of the carbocycle (e.g. bicyclo[1.1.1]pentane, bicyclo [2.2.1] heptane and bicyclo [3.2.1] octane).

As used herein the term “fused bicyclic ring system” or “fused carbobicyclic ring system” refers to a carbocycle connected at two non-adjacent ring atoms of the carbocycle. Fused bicyclic ring systems include, but are not limited to, 1,2,3,4-tetrahydronaphthalene, (1S,5R)-1-methylbicyclo[3.1.0]hexane, bicyclo[3.1.0]hexane, bicyclo[4.1.0]heptane and 2,3-dihydro-1H-indene.

As used herein the term “spiro carbocyclic ring” means a two-ring system wherein both rings share one common carbon atom. Examples of spiro carbocyclic rings include spiro[2.5]octane, spiro[2.3]hexane, spiro[2.4]heptane, spiro[3.4]octane and the like. “Halogen” or “halo” may be fluorine, chlorine, bromine or iodine (preferred halogens as substituents are fluorine and chlorine).

As used herein, the term “haloalkyl” or “halo-substituted alkyl” or refers to an alkyl group as defined herein, wherein at least one of the hydrogen atoms is replaced by a halo atom. The haloalkyl group can be monohalo-alkyl, dihaloalkyl or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Typically the polyhaloalkyl group contains up to 9, or 8, or 7, or 6, or 5, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl group refers to an alkyl group having all hydrogen atoms replaced with halo atoms.

As used herein, the term “heteroaryl” refers to an aromatic 5- to 6-membered monocyclic or an 8- to 10-membered bicyclic ring system, having 1 to 4 heteroatoms independently selected from O, N and S, and wherein N can be oxidized (e.g., N(O)) or quaternized, and S can be optionally oxidized to sulfoxide and sulfone.

Examples of “5 or 6 membered heteroaryl” or “5- to 6-membered monocyclic heteroaryl” include, but are not limited to, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyrazinyl, pyrimidinyl, pyridazinyl, triazinyl, tetrazinyl, and the like. In some embodiments, a 5 to 6 membered heteroaryl is selected from pyrrolyl, pyridyl, pyrazolyl, thienyl, furanyl, oxazolyl, isoxazolyl, isothiazolyl, thiazolyl, imidazolyl, tetrazolyl, triazinyl, pyrimidyl, pyrazinyl, and thiazolyl. In some embodiments, a 5 to 6 membered heteroaryl is selected from pyridinyl, pyrimidinyl, 2H-1,2,3-triazolyl, isoxazolyl, isothiazolyl, thiazolyl, pyrazolyl and thienyl.

Examples of a 5-membered heteroaryl include, but are not limited to, pyrazolyl, imidazolyl, oxazolyl, isoxazolyl, 1,2,3-oxadiazolyl, 1,3,4-oxadiazolyl, 1,2,4-oxadizolyl, 1,2,3-thiadiazolyl, 1,3,4-thiadiazolyl, 1,2,4-thiadiazolyl, 1,2,3-triazolyl, 1,2,4-triazolyl, and tetrazolyl. Examples of 8- to 10-membered bicyclic heteroaryls include, but are not limited to, imidazolthiazolyl, imidazopyridinyl, imidazo[1,2-a]pyridinyl, imidazo[2,1-b]thiazolyl, indazolyl, 2H-indazolyl, indolyl, isoindolyl, 2λ²-isoindolinyl, benzimidazolyl, benzofuranyl, benzothiophenyl, benzothiazolyl, quinolinyl, isoquinolinyl, quinazolinyl, purinyl, thienopyridinyl and thieno[3,2-b]pyridinyl. Examples of 9- to 10-membered bicyclic heteroaryls include, but are not limited to, imidazopyridinyl, imidazo[1,2-a]pyridinyl, indazolyl, 2H-indazolyl, indolyl, isoindolyl, 2λ²-isoindolinyl, benzimidazolyl, benzofuranyl, benzothiophenyl, benzothiazolyl, quinolinyl, isoquinolinyl, quinazolinyl, purinyl, thienopyridinyl and thieno[3,2-b]pyridinyl.

In some embodiments, a 5-membered heteroaryl is selected from

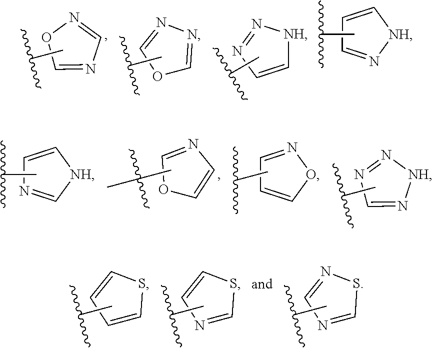

In some embodiments, a 6-membered heteroaryl is selected from

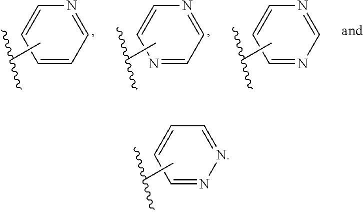

Examples of 9 to 10 membered heteroaryls include indolyl, indazolyl, benzofuranyl, quinoxalinyl, pyrazolo[1,5-a]pyridinyl, [1,2,4]triazolo[4,3-a]pyridinyl, isothiazolo[4,3-b]pyridinyl, pyrazolo[1,5-a]pyrimidinyl, pyrido[3,2-d]pyrimidinyl, imidazo[1,2-b]pyridazinyl, thieno[2,3-b]pyrazinyl, 1H-benzo[d]imidazolyl, benzo[d]thiazolyl, 1,6-naphthyridinyl, and 1,5-naphthyridinyl. In some embodiments, a 9 to 10 membered heteroaryl is selected from pyrazolo[1,5-a]pyridinyl, [1,2,4]triazolo[4,3-a]pyridinyl, isothiazolo[4,3-b]pyridinyl, pyrazolo[1,5-a]pyrimidinyl, pyrido[3,2-d]pyrimidinyl, imidazo[1,2-b]pyridazinyl, thieno[2,3-b]pyrazinyl, 1H-benzo[d]imidazolyl, benzo[d]thiazolyl, 1,6-naphthyridinyl, 1,5-naphthyridinyl, and 2H-indazolyl.

In some embodiments, a heteroaryl is an 8- to 9-membered bicyclic heteroaryl selected from:

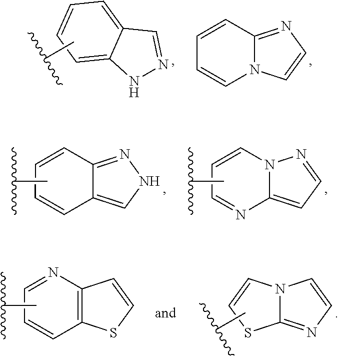

The term “heterocycle” or “monocyclic heterocycle” refers to a monocyclic ring which is partially or fully saturated and contains 1 to 2 heteroatoms, independently selected from sulfur, oxygen and/or nitrogen. Monocyclic heterocycles include, but are not limited to, oxtanyl, tetrahydrofuranyl, dihydrofuranyl, 1,4-dioxanyl, morpholinyl, 1,4-dithianyl, piperazinyl, piperidinyl, 1,3-dioxolanyl, pyrrolinyl, pyrrolidinyl, tetrahydropyranyl, oxathiolanyl, dithiolanyl, 1,3-dioxanyl, 1,3-dithianyl, oxathianyl, thiomorpholinyl, thiomorpholinyl 1,1 dioxide, tetrahydro-thiopyran 1,1-dioxide, 1,4-diazepanyl.

In some embodiments, a monocyclic heterocycle is selected from:

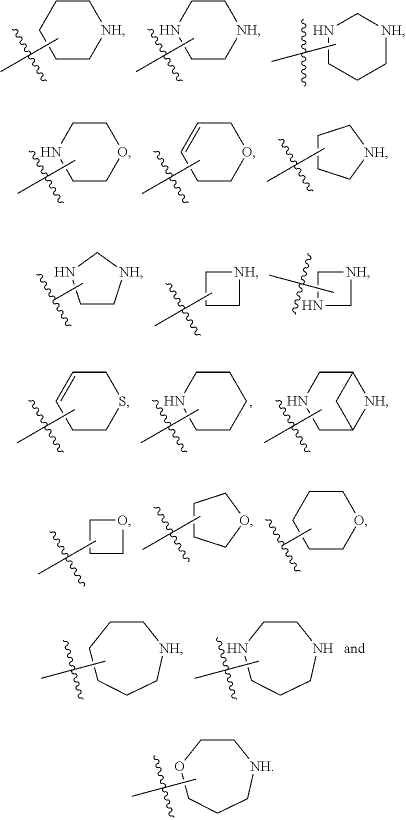

The term “bicyclic heterocycle” refers to a bicyclic ring which is partially or fully saturated and contains 1 to 2 heteroatoms, independently selected from sulfur, oxygen and/or nitrogen. Bicyclic heterocycles include, but are not limited to, 2,6-diazaspiro[3.3]heptane.

The term “partially or fully saturated heterocycle” refers to a nonaromatic ring that is either partially or fully saturated and may exist as a single ring, bicyclic ring (including fused heterocyclic rings) or a spiro ring. Unless specified otherwise, the heterocyclic ring is generally a 3 to 7 membered ring containing 1 to 3 heteroatoms (preferably 1, 2 or 3 heteroatoms) independently selected from sulfur, oxygen and/or nitrogen. Partially saturated or fully saturated heterocyclic rings include groups such as epoxy, aziridinyl, azetidinyl, tetrahydrofuranyl, dihydrofuranyl, dihydropyridinyl, pyrrolidinyl, imidazolidinyl, imidazolinyl, 1H-dihydroimidazolyl, hexahydropyrimidinyl, piperidinyl, piperazinyl, pyrazolidinyl, 2H-pyranyl, 4H-pyranyl, oxazinyl, morpholino, thiomorpholino, tetrahydrothienyl, tetrahydrothienyl 1,1-dioxide, oxazolidinyl, thiazolidinyl, 7-oxabicyclo[2.2.1]heptane, and the like. A partially saturated heterocyclic ring also includes groups wherein a heterocyclic ring is fused to an aryl or heteroaryl ring (e.g., 2,3-dihydrobenzo furanyl, indolinyl (or 2,3-dihydroindolyl), 2,3-dihydrobenzothiophenyl, 2,3-dihydro benzothiazolyl, 1,3-dihydro-2H-benzo[d]imidazol-2-one, 1,2,3,4-tetrahydro quinolinyl, 1,2,3,4-tetrahydroisoquinolinyl, 5,6,7,8-tetrahydropyrido[3,4-b]pyrazinyl).

In some embodiments, a partially or fully saturated heterocycle is selected from:

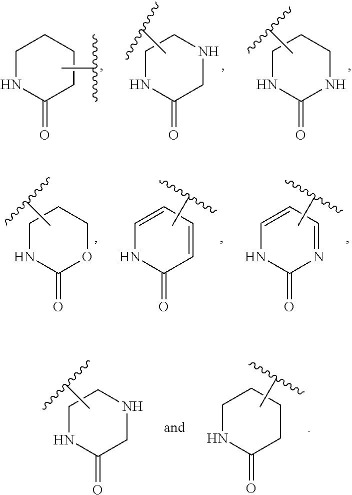

As used herein the term “bridged-heterocyclic ring system” refers to a 5 to 10 membered heterobicyclic moiety connected at two non-adjacent ring atoms of the heterocycle containing at least one heteroatom (e.g., oxygen, sulfur, nitrogen or combinations thereof) within a 5 to 10 membered cyclic ring system. Examples of the “bridged-heterocyclic ring system” include, but are not limited to, 2-oxabicyclo[2.1.1]hexane, 3-oxabicyclo[4.1.0]heptane, 2-oxabicyclo[2.2.1]heptane, 2-oxabicyclo[2.2.2]octane, 8-oxabicyclo[3.2.1]octane, and 2,6-dioxabicyclo[3.2.1]octane.

As used herein the term “fused heterobicyclic ring system” refers to two ring systems that share two adjacent ring atoms and at least one of the rings containing a ring atom that is a heteroatom selected from O, N and S. Examples of fused heterobicylic ring systems include, but are not limited to, 1,3-dihydroisobenzofuran, 4-methyl-3,4-dihydro-2H-benzo[b][1,4]oxazine, pyrazolo[1,5-a]pyrimidine, 5,6-dihydro-4H-pyrrolo[1,2-b]pyrazole, 6,7-dihydro-5H-cyclopenta[b]pyridine, 2-oxabicyclo[2.1.0]pentane, indolin-2-one, 2,3-dihydrobenzofuran, 1-methyl-2-oxo-1,2,3,4-tetrahydroquinoline, 3,4-dihydroquinolin-2(1H)-one, chromane, isochromane, 4,5,6,7-tetrahydro-3H-imidazo[4,5-c]pyridine, 8-azabicyclo [3.2.1]octan-3-ol, octahydropyrrolo[1,2-a]pyrazine, 5,6,7,8-tetrahydroimidazo[1,2-a]pyrazine, 3,8 diazabicyclo[3.2.1]octane, 8-oxa-3-azabicyclo[3.2.1]octane, 7-oxabicyclo [2.2.1]heptane, 1H-pyrazole, 2,5-diazabicyclo[2.2.1]heptane, 5,6,7,8-tetrahydro-[1,2,4]triazolo[4,3-a]pyrazine, 3-oxabicyclo[3.1.0]hexane, or 3-azabicyclo[3.1.0]hexane. A partially saturated heterocyclic ring also includes groups wherein the heterocyclic ring is fused to an aryl or heteroaryl ring (e.g., 2,3-dihydrobenzofuranyl, indolinyl (or 2,3-dihydro indolyl), 2,3-dihydrobenzothiophenyl, 2,3-dihydrobenzothiazolyl, 1,2,3,4-tetrahydro quinolinyl, 1,2,3,4-tetrahydroisoquinolinyl, 5,6,7,8-tetrahydropyrido[3,4-b]pyrazinyl, 6,7-dihydro-5H-pyrazolo[5,1-b][1,3]oxazine, and the like. In some embodiments, “fused heterobicyclic ring system” refers fused bicyclic heteoaryl.

In some embodiments, the term “7 to 10 membered fused heterobicyclic ring system” is limited to a 7 to 10 membered bicyclic heteroaryl, such as pyrazolo[1,5-a]pyrimidine, pyrazolo[1,5-a]pyridine, [1,2,4]triazolo[4,3-a]pyridine, [1,2,4]triazolo[1,5-a]pyridine, isothiazolo[4,3-b]pyridine, pyrrolo[1,2-a]pyrimidine, pyrido[3,2-d]pyrimidine, imidazo[1,2-b]pyridazine, thieno[2,3-b]pyrazine, 1H-benzo[d]imidazole, benzo[d]thiazole, 1,6-naphthyridine and 1,5-naphthyridine.

As used herein the term “spiro heterobicyclic ring system” means a two-ring system wherein both rings share one common atom. Examples of spiro heterobicyclic ring systems include oxaspiro[2.4]heptanyl, 5-oxaspiro[2.4]heptanyl, 4-oxaspiro[2.4]heptane, 4-oxaspiro[2.5]octanyl, 6-oxaspiro[2.5]octanyl, oxaspiro[2.5]octanyl, oxaspiro[3.4]octanyl, oxaspiro[bicyclo[2.1.1]hexane-2,3′-oxetan]-1-yl, oxaspiro[bicyclo[3.2.0]heptane-6,1′-cyclobutan]-7-yl, 2,6-diazaspiro[3.3]heptanyl, -oxa-6-azaspiro[3.3]heptane, 2,2,6-diazaspiro[3.3]heptane, 3-azaspiro[5.5]undecanyl, 3,9-diazaspiro[5.5]undecanyl, 7-azaspiro[3.5]nonane, 2,6-diazaspiro[3.4]octane, 8-azaspiro[4.5]decane, 1,6-diazaspiro[3.3]heptane, 5-azaspiro[2.5]octane, 4,7-diazaspiro[2.5]octane, 5-oxa-2-azaspiro[3.4]octane, 6-oxa-1-azaspiro[3.3]heptane, 3-azaspiro[5.5]undecanyl, 3,9-diazaspiro[5.5]undecanyl, and the like.

As used herein “Hydroxyl” or “Hydroxy” refers to the group —OH.

The term “oxo” (═O) refers to an oxygen atom connected to a carbon or sulfur atom by a double bond. Examples include carbonyl, sulfinyl, or sulfonyl groups (—C(O)—, —S(O)— or —S(O)₂—) such as, a ketone, aldehyde, or part of an acid, ester, amide, lactone, or lactam group and the like.

As used herein, when a group/variable (e.g., L, Z1, Z2 etc.) is defined as “bond”, it means that the two moieties attached to the group/variable are connected directly to each other. For example, when L in Formula (A) is a bond, it means that the IRAK moiety and the DSM moiety are connected directly.

IRAK-L-DSM (A).

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

Unless specified otherwise, the term “compounds of the present disclosure” refers to compounds of formula (A), as well as all stereoisomers (including diastereoisomers and enantiomers), rotamers, tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound of the disclosure. “Salts” include in particular “pharmaceutical acceptable salts”.

The term “pharmaceutically acceptable salts” refers to salts that retain the biological effectiveness and properties of the compounds of this disclosure and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

In some embodiments, the disclosure provides deuterated compounds in which any or more positions occupied by hydrogen can include enrichment by deuterium above the natural abundance of deuterium. For example, one or more hydrogen atoms are replaced with deuterium at an abundance that is at least 3340 times greater than the natural abundance of deuterium, which is 0.015% (i.e., at least 50.1% incorporation of deuterium), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In one embodiment, hydrogen is present at all positions at its natural abundance.

Isotopically-labeled compounds of formula (A) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the disclosure include those wherein the solvent of crystallization may be isotopically substituted, e.g. D₂O, d₆-acetone, d₆-DMSO.

It will be recognized by those skilled in the art that the compounds of the present disclosure may contain chiral centers and as such may exist in different stereoisomeric forms. As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1RS,2RS) as a racemic mixture of (1R,2R) and (1S,2S); (1RS,2SR) as a racemic mixture of (1R,2S) and (1S,2R)).

“Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “inhibit”, “inhibition” or “inhibiting” refers to the reduction or suppression of a given condition, symptom, or disorder, or disease, or a significant decrease in the baseline activity of a biological activity or process.

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

The phrase “pharmaceutically acceptable” indicates that the substance, composition or dosage form must be compatible chemically and/or toxicologically, with the other ingredients comprising a formulation, and/or the mammal being treated therewith.

As used herein, the term “treat”, “treating” or “treatment” of any disease or disorder, refers to the management and care of a patient for the purpose of combating the disease, condition, or disorder and includes the administration of a compound of the present disclosure to prevent the onset of the symptoms or complications, alleviating the symptoms or complications, or eliminating the disease, condition or disorder.

As used herein the term “stroke” has the meaning normally accepted in the art. The term can broadly refer to the development of neurological deficits associated with the impaired blood flow regardless of cause. Potential causes include, but are not limited to, thrombosis, hemorrhage and embolism. The term “ischemic stroke” refers more specifically to a type of stroke that is of limited extent and caused due to a blockage of blood flow.

As used herein, a subject is “in need of” a treatment if such subject would benefit biologically, medically or in quality of life from such treatment (preferably, a human).

As used herein the term “co-administer” refers to the presence of two active agents in the blood of an individual. Active agents that are co-administered can be concurrently or sequentially delivered.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

### II. Compounds of the Disclosure

The compounds of the present disclosure comprise a degradation signaling moiety (DSM) that can bind to an E3 ligase (e.g., the cereblon protein), an IRAK binding or targeting moiety and optionally a Linker that covalently links the DSM to the IRAK binding or targeting moiety.

In a first embodiment, the compound of the present disclosure is a compound of Formula (A):

IRAK-L-DSM (A),

or a pharmaceutically acceptable salt thereof, wherein the IRAK, L and DSM portions in Formula (A) as as described in the first aspect above. In some embodiments, the DSM, IRAK and Linker portions in Formula (A) are as described below.

### A. IRAK4 Binding or Targeting Moiety

In a second embodiment of the present disclosure, for the compound of formula (A), IRAK is an IRAK4 binding moiety represented by Formula (IA) or (IB):

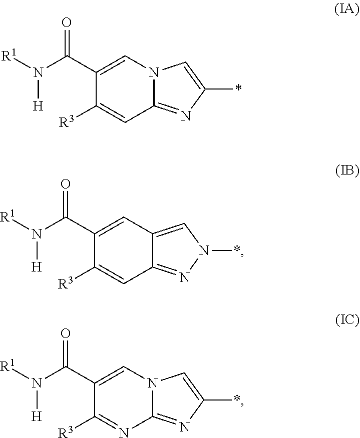

or a pharmaceutically acceptable salt thereof, and the definitions for the other variables are as defined in the first embodiment.

In a third embodiment of the present disclosure, for the compound of formula (A), IRAK is an IRAK4 binding moiety represented by Formula (IA) or (IB):

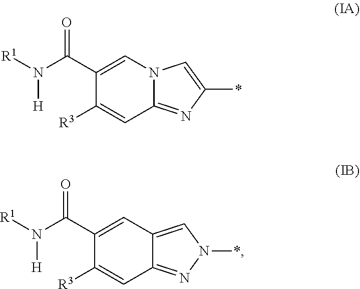

or a pharmaceutically acceptable salt thereof, and the definitions for the other variables are as defined in the first embodiment.

In a fourth embodiment of the present disclosure, for the compound of formula (A), or a pharmaceutically acceptable salt thereof, IRAK is an IRAK4 binding moiety represented by one of Formula (I), (IA), (IB), or (IC) wherein R¹ is selected from phenyl optionally substituted with 1 to 3 R⁵; 5 or 6 membered heteroaryl having 1 to 2 nitrogen atoms, said heteroaryl is optionally substituted with 1 to 3 R⁵; 5 or 6 membered partially or fully saturated heterocycle having 1 to 2 heteroatoms independently selected from oxygen and nitrogen, said heterocycle may be optionally substituted with 1 to 3 R⁵; and 9 to 10 membered bicyclic heteroaryl having 1, 2 or 3 nitrogen atoms, said ring system is optionally substituted with 1 to 3 R⁵; and the definitions for the other variables are as defined in the first, second, or third embodiment.

In a fifth embodiment of the present disclosure, for the compound of formula (A), or a pharmaceutically acceptable salt thereof, IRAK is an IRAK4 binding moiety represented by one of Formula (I), (IA), (IB), or (IC) wherein R¹ is selected from phenyl optionally substituted with 1 to 2 R⁵; pyrazole optionally substituted with 1 to 2 R⁵; pyridine optionally substituted with 1 to 2 R⁵; pyridone optionally substituted with 1 to 2 R⁵; pyrimidine optionally substituted with 1 to 2 R⁵; and pyrazolo[1,5-a]pyrimidine optionally substituted with 1 to 2 R⁵; and the definitions for the other variables are as defined in the first, second, or third embodiment.

In a sixth embodiment of the present disclosure, for the compound of formula (A), or a pharmaceutically acceptable salt thereof, IRAK is an IRAK4 binding moiety represented by one of Formula (I), (IA), (IB), or (IC) wherein R¹ is selected from phenyl optionally substituted with 1 to 2 R⁵; pyrazole optionally substituted with 1 to 2 R⁵; pyridine optionally substituted with 1 to 2 R⁵; pyrimidine optionally substituted with 1 to 2 R⁵; and pyrazolo[1,5-a]pyrimidine optionally substituted with 1 to 2 R⁵; and the definitions for the other variables are as defined in the first, second, or third embodiment.

In a seventh embodiment of the present disclosure, for the compound of formula (A), or a pharmaceutically acceptable salt thereof, IRAK is an IRAK4 binding moiety represented by one of Formula (I), (IA), (IB), or (IC) wherein R¹ is represented by one of the following formulae:

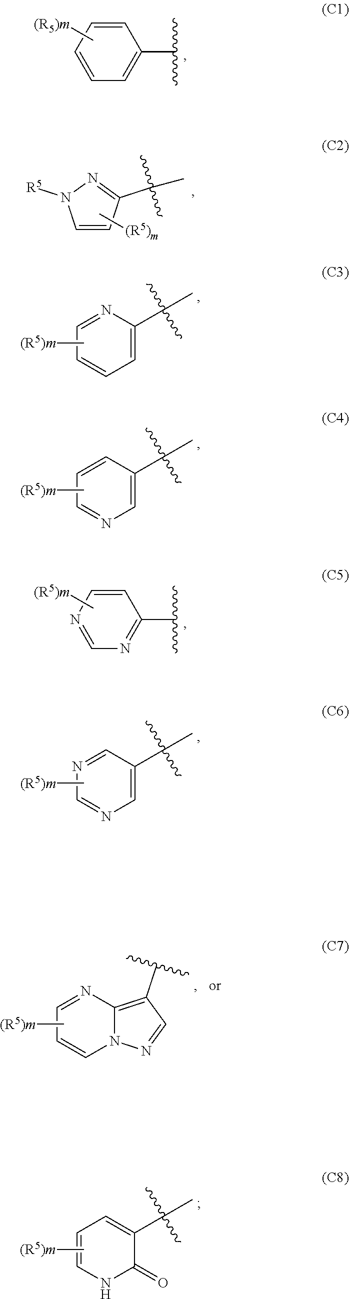

wherein m is 0, 1 or 2; and the definitions for the other variables are as defined in the first, second, or third embodiment.

In an eighth embodiment of the present disclosure, for the compound of formula (A), or a pharmaceutically acceptable salt thereof, IRAK is an IRAK4 binding moiety represented by one of Formula (I), (IA), (IB), or (IC) wherein R¹ is represented by one of the following formulae:

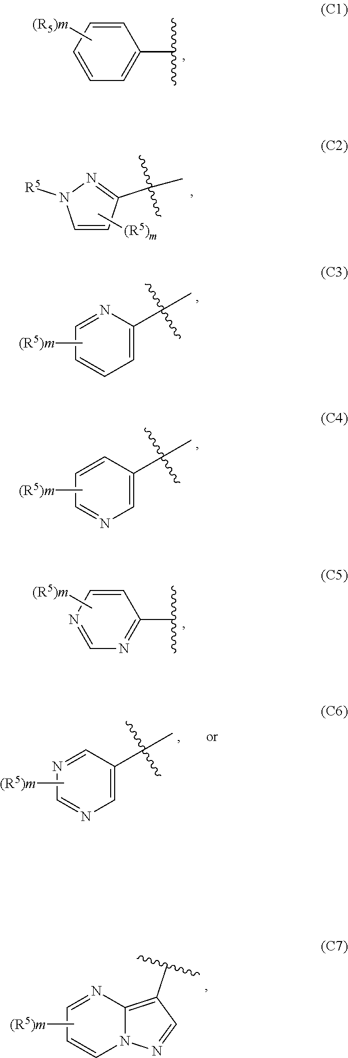

wherein m is 0, 1 or 2; and the definitions for the other variables are as defined in the first, second, or third embodiment.

In a ninth embodiment of the present disclosure, for the compound of formula (A), or a pharmaceutically acceptable salt thereof, IRAK is an IRAK4 binding moiety represented by one of Formula (I), (IA), (IB), or (IC) wherein R¹ is represented by one of the following formulae:

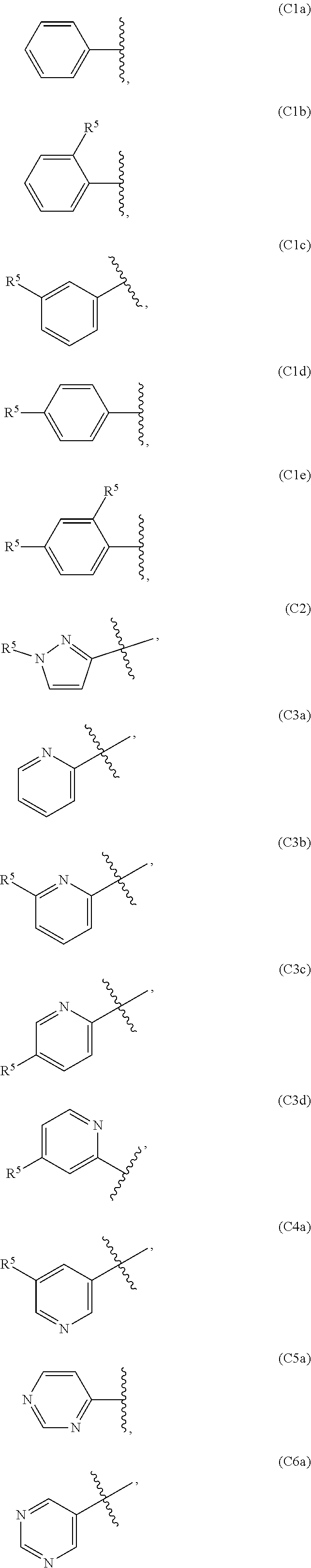

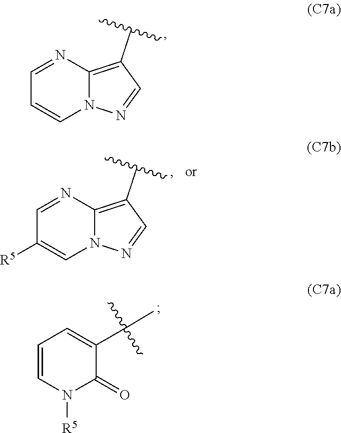

and the definitions for the other variables are as defined in the first, second, or third, embodiment.

In a tenth embodiment of the present disclosure, for the compound of formula (A), or a pharmaceutically acceptable salt thereof, IRAK is an IRAK4 binding moiety represented by one of Formula (I), (IA), (IB), or (IC) wherein R¹ is represented by one of the following formulae:

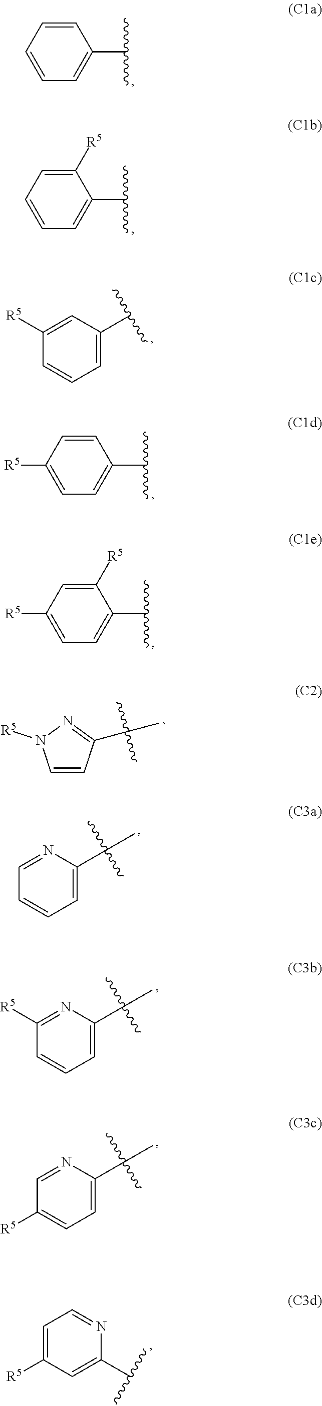

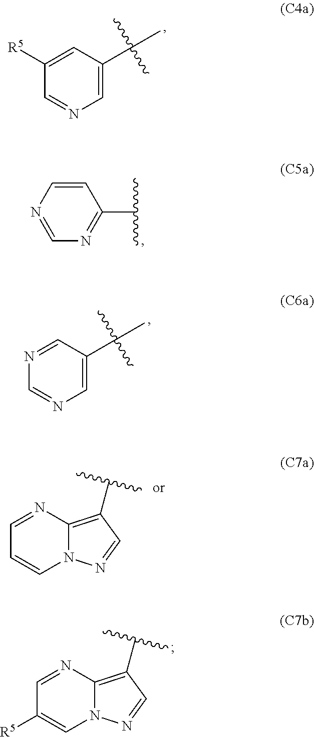

and the definitions for the other variables are as defined in the first, second, or third, embodiment.

In an eleventh embodiment of the present disclosure, for the compound of formula (A), or a pharmaceutically acceptable salt thereof, IRAK is an IRAK4 binding moiety represented by one of Formula (I), (IA), (IB), or (IC) wherein R¹ is represented by one of the following formulae:

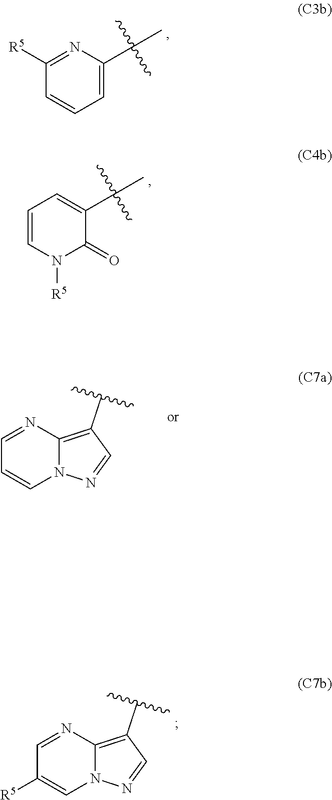

and the definitions for the other variables are as defined in the first, second, or third embodiment.

In a twelfth embodiment of the present disclosure, for the compound of formula (A), or a pharmaceutically acceptable salt thereof, IRAK is an IRAK4 binding moiety represented by one of Formula (I), (IA), (IB), or (IC) wherein R¹ is represented by one of the following formulae:

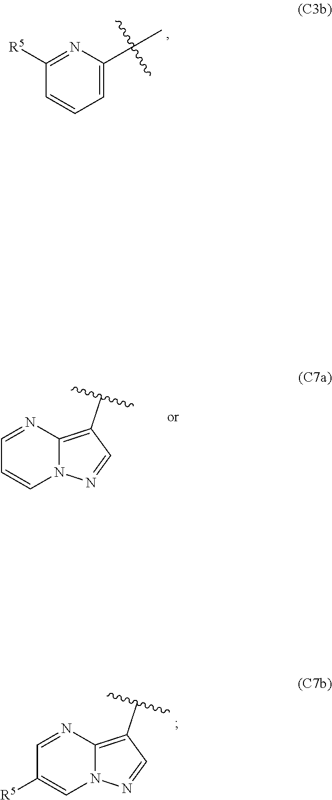

and the definitions for the other variables are as defined in the first, second, or third embodiment.

In a thirteenth embodiment of the present disclosure, for the compound of formula (A), or a pharmaceutically acceptable salt thereof, IRAK is an IRAK4 binding moiety represented by one of Formula (I), (IA), (IB), or (IC) wherein R² is hydrogen; and the definitions for the other variables are as defined in the first, second, third, fourth, fifth, sixth, seventh, eighth, ninth, tenth, eleventh, or twelfth embodiment.

In a fourteenth embodiment of the present disclosure, for the compound of formula (A), or a pharmaceutically acceptable salt thereof, IRAK is an IRAK4 binding moiety represented by one of the following formulae:

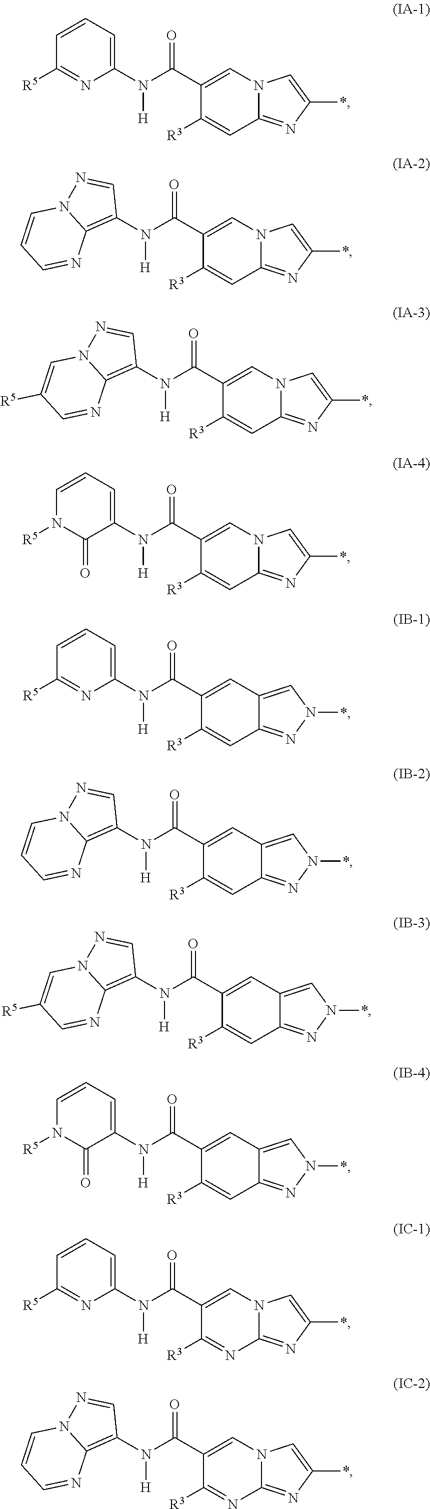

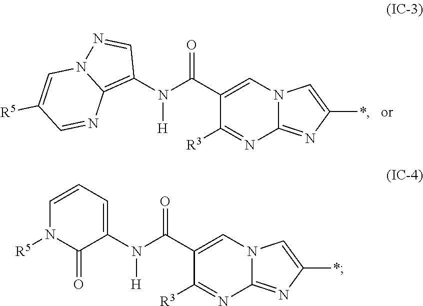

and the definitions for the other variables are as defined in the first embodiment.

In a fifteenth embodiment of the present disclosure, for the compound of formula (A), or a pharmaceutically acceptable salt thereof, IRAK is an IRAK4 binding moiety represented by one of the following formulae:

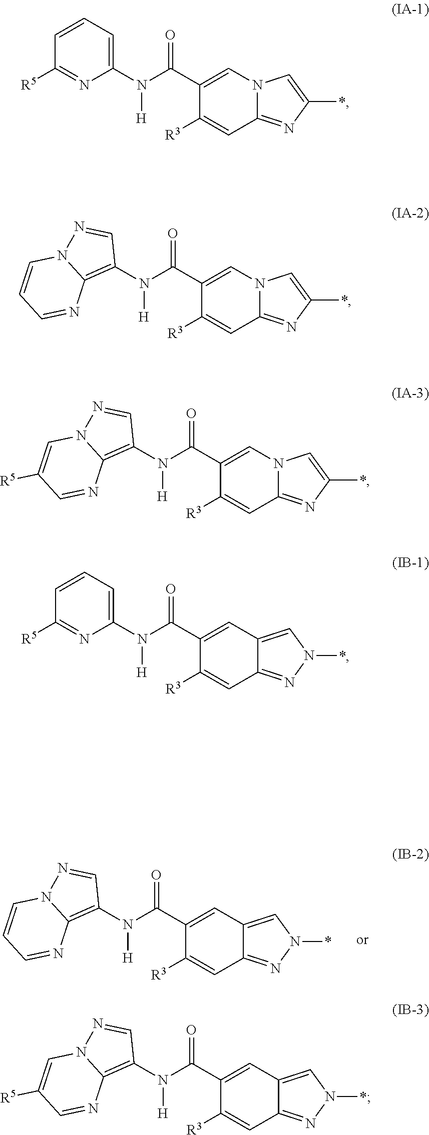

and the definitions for the other variables are as defined in the first embodiment.

In a sixteenth embodiment of the present disclosure, for the compound of formula (A), or a pharmaceutically acceptable salt thereof, IRAK is an IRAK4 binding moiety represented by one of Formula (I), (IA), (IB), (IC), (IA-1), (IA-2), (IA-3), (IA-4), (IB-1), (IB-2), (IB-3), (IB-4), (IC-1), (IC-2), (IC-3), or (IC-4), wherein R³ is C₁₋₄alkyl or —OR⁶, wherein the C₁₋₄alkyl is optionally substituted with at least one halogen; and R⁶ is C₁₋₅alkyl; and the definitions for the other variables are as defined in the first, second, third, fourth, fifth, sixth, seventh, eighth, ninth, tenth, eleventh, twelfth, thirteenth, fourteenth, or fifteenth embodiment.

In a seventeenth embodiment of the present disclosure, for the compound of formula (A), or a pharmaceutically acceptable salt thereof, IRAK is an IRAK4 binding moiety represented by one of Formula (I), (IA), (IB), (IC), (IA-1), (IA-2), (IA-3), (IA-4), (IB-1), (IB-2), (IB-3), (IB-4), (IC-1), (IC-2), (IC-3), or (IC-4), wherein R³ is —CF₃ or —O—CH(CH₃)₂; and the definitions for the other variables are as defined in the first, second, third, fourth, fifth, sixth, seventh, eighth, ninth, tenth, eleventh, twelfth, thirteenth, fourteenth, or fifteenth embodiment.

In an eighteenth embodiment of the present disclosure, for the compound of formula (A), or a pharmaceutically acceptable salt thereof, IRAK is an IRAK4 binding moiety represented by one of Formula (I), (IA), (IB), (IC), (IA-1), (IA-2), (IA-3), (IA-4), (IB-1), (IB-2), (IB-3), (IB-4), (IC-1), (IC-2), (IC-3), or (IC-4), wherein R³ is —O—CH(CH₃)₂; and the definitions for the other variables are as defined in the first, second, third, fourth, fifth, sixth, seventh, eighth, ninth, tenth, eleventh, twelfth, thirteenth, fourteenth, or fifteenth embodiment.

In a nineteenth embodiment of the present disclosure, for the compound of formula (A), or a pharmaceutically acceptable salt thereof, IRAK is an IRAK4 binding moiety represented by one of Formula (I), (IA), (IB), (IC), (IA-1), (IA-2), (IA-3), (IA-4), (IB-1), (IB-2), (IB-3), (IB-4), (IC-1), (IC-2), (IC-3), or (IC-4), wherein R⁵ for each occurrence, is independently selected from C₁₋₄ alkyl, halogen, C₁₋₄ haloalkyl, and C₃₋₄cycloalkyl, and wherein said C₃₋₄cycloalkyl is optionally substituted with 1 halo; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, or eighteenth embodiment.

In a twentieth embodiment of the present disclosure, for the compound of formula (A), or a pharmaceutically acceptable salt thereof, IRAK is an IRAK4 binding moiety represented by one of Formula (I), (IA), (IB), (IC), (IA-1), (IA-2), (IA-3), (IA-4), (IB-1), (IB-2), (IB-3), (IB-4), (IC-1), (IC-2), (IC-3), or (IC-4), wherein R⁵ for each occurrence, is independently selected from C₁₋₄ alkyl, halogen, and C₁₋₄ haloalkyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, or eighteenth embodiment.

In a twenty-first embodiment of the present disclosure, for the compound of formula (A), or a pharmaceutically acceptable salt thereof, IRAK is an IRAK4 binding moiety represented by one of Formula (I), (IA), (IB), (IC), (IA-1), (IA-2), (IA-3), (IA-4), (IB-1), (IB-2), (IB-3), (IB-4), (IC-1), (IC-2), (IC-3), or (IC-4), wherein R⁵ for each occurrence, is independently selected from —CH₃, —CHF₂, —CF₃, F, cyclopropyl, and

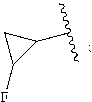 [SMILES: *C1CC1F]

and the definitions for the other variables are as defined nineteenth or twentieth embodiment.

In a twenty-second embodiment of the present disclosure, for the compound of formula (A), or a pharmaceutically acceptable salt thereof, IRAK is an IRAK4 binding moiety represented by one of Formula (I), (IA), (IB), (IC), (IA-1), (IA-2), (IA-3), (IA-4), (IB-1), (IB-2), (IB-3), (IB-4), (IC-1), (IC-2), (IC-3), or (IC-4), wherein R⁵ for each occurrence, is independently selected from —CH₃, —CHF₂, —CF₃ and F; and the definitions for the other variables are as defined in the nineteenth or twentieth embodiment.

In a twenty-third embodiment of the present disclosure, for the compound of formula (A), or a pharmaceutically acceptable salt thereof, IRAK is an IRAK4 binding moiety represented by one of the following formulae:

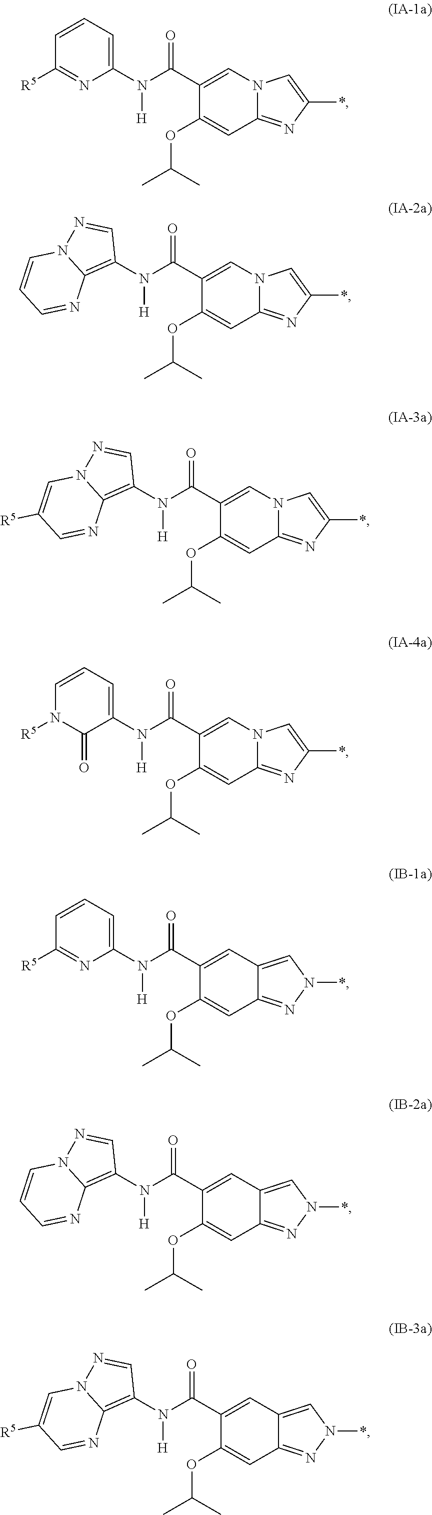

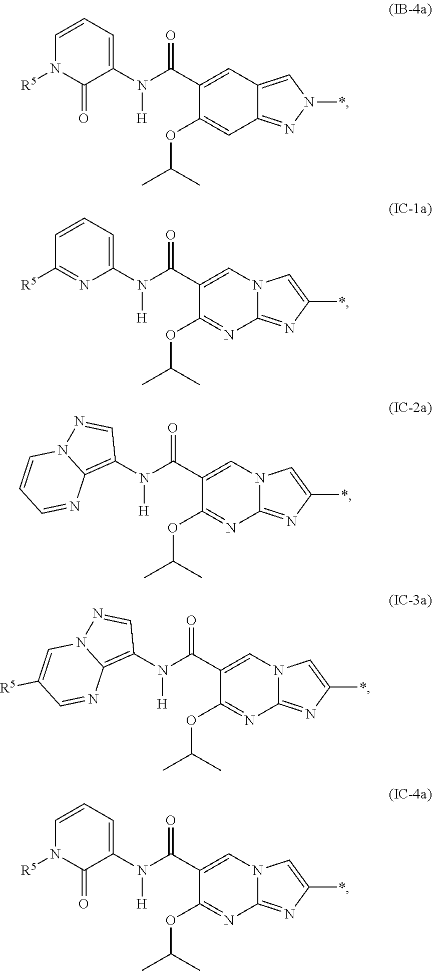

wherein R⁵ is C₁₋₃ alkyl or C₁₋₃ haloalkyl or C₃₋₄cycloalkyl, and wherein said C₃₋₄ cycloalkyl is optionally substituted with 1 halo; and the definitions for the other variables are as defined in the first embodiment.

In a twenty-fourth embodiment of the present disclosure, for the compound of formula (A), or a pharmaceutically acceptable salt thereof, IRAK is an IRAK4 binding moiety represented by one of the following formulae:

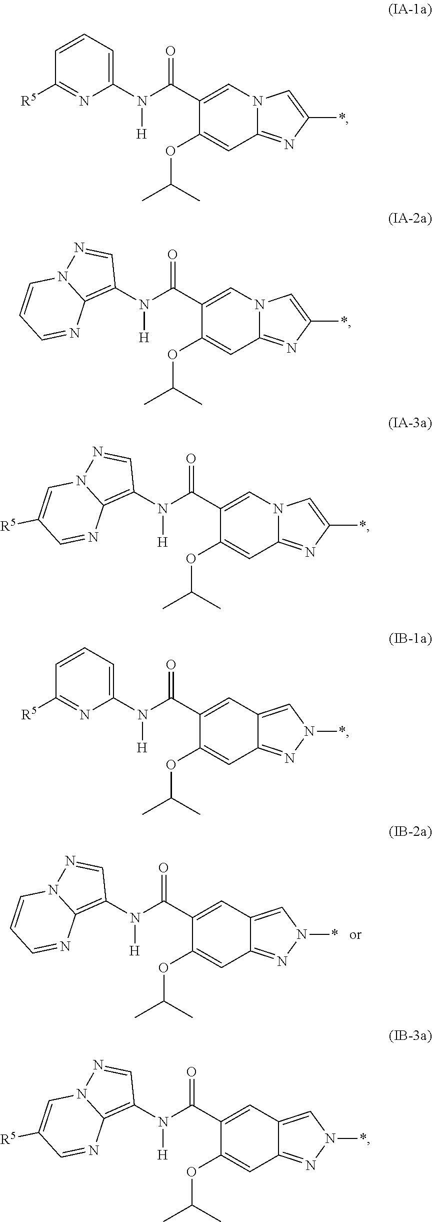

wherein R⁵ is C₁₋₃ alkyl or C₁₋₃ haloalkyl; and the definitions for the other variables are as defined in the first embodiment.

In a twenty-fifth embodiment of the present disclosure, for the compound of formula (A), or a pharmaceutically acceptable salt thereof, IRAK is an IRAK4 binding moiety represented by one of Formula (IA-1a), (IA-2a), (IA-3a), (IA-4a), (IB-1a), (IB-2a), (IB-3a), (IB-4a), (IC-1a), (IC-2a), (IC-3a), or (IC-4a), wherein R⁵ is CH₃, CHF₂, CF₃, cyclopropyl, or

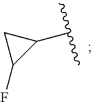 [SMILES: *C1CC1F]

and the definitions for the other variables are as defined in the twenty-third or twenty-fourth embodiment.

In a twenty-sixth embodiment of the present disclosure, for the compound of formula (A), or a pharmaceutically acceptable salt thereof, IRAK is an IRAK4 binding moiety represented by one of Formula (IA-1a), (IA-2a), (IA-3a), (IA-4a), (IB-1a), (IB-2a), (IB-3a), (IB-4a), (IC-1a), (IC-2a), (IC-3a), or (IC-4a), wherein R⁵ is CH₃, CHF₂ or CF₃; and the definitions for the other variables are as defined in the twenty-third or twenty-fourth embodiment.

### B. Degradation Signaling Moiety (DSM)

The degradation signaling moiety (DSM) in compounds of formula (A) or a pharmaceutically acceptable salt thereof can be a suitable moiety that binds to an E3 ubiquitin ligase (e.g., the cereblon protein), for example, a degron or E3 ubiquitin ligase binding or targeting moiety described in WO2020/210630 titled “Tricyclic Degraders of Ikaros and Aiolos”; WO2020/181232 titled “Heterocyclic Compounds for Medical Treatment”; WO2020/132561 titled “Targeted Protein Degradation”; WO2019/204354 titled “Spirocyclic Compounds”; WO2019/099868 titled “Degraders and Degrons for Targeted Protein Degradation”; WO2018/237026 titled “N/O-Linked Degrons and Degronimers for Protein Degradation”; WO2017/197051 titled “Amine-Linked C3-Glutarimide Degronimers for Target Protein Degradation”; WO2017/197055 titled “Heterocyclic Degronimers for Target Protein Degradation”; WO2017/197036 titled “Spirocyclic Degronimers for Target Protein Degradation”; WO2017/197046 titled “C3-Carbon Linked Glutarimide Degronimers for Target Protein Degradation”; and WO2017/197056 titled “Bromodomain Targeting Degronimers for Target Protein Degradation”. Other degradation signaling moiety or E3 ubiquitin ligase binding or targeting moiety that can be used are those described in WO2015/160845; WO2016/105518; WO2016/118666; WO2016/149668; WO2016/197032; WO2016/197114; WO2017/007612; WO2017/011371; WO2017/011590; WO2017/030814; WO2017/046036; WO2017/176708; WO2017/176957; WO2017/180417; WO2018/053354; WO2018/071606; WO2018/102067; WO2018/102725; WO2018/118598; WO2018/119357; WO2018/119441; WO2018/119448; WO2018/140809; WO2018/144649; WO2018/119448; WO2018/226542; WO2019/023553, WO2019/195201, WO2019/199816, and WO2019/099926. The entire teachings of the above-referenced PCT publications are incorporated herein by reference.

In a twenty-seventh embodiment of the present disclosure, for the compound of formula (A), or a pharmaceutically acceptable salt thereof, DSM is a degradation signaling moiety of formula (D).

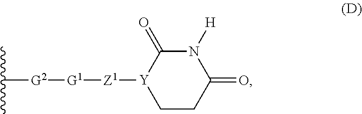

wherein

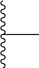

represents a bond to the linker L; Y is CR^{D1} or N; Z¹ is selected from a bond, —NR^{D2}—, —O— and —CH₂—; G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more (e.g., 1 to 6, 1 to 3, or 1, 2, 3, 4, 5 or 6) R^{D3}; G² is selected from Het₁, *NR^{D4}—C₄₋₆ cycloalkyl-*, *—NR^{D4}-Het₁-, *—NR^{D4}-Het₁-C₁₋₄ alkyl-*, *—C₁₋₄ alkyl-C(R^{D1})=Het₁-2, *—C(O)—C₁₋₄ alkyl-Het₁-*, *-Het₁-C₁₋₆ alkyl-*, *-Het₁-O—*, *—C(O)—C₁₋₄ alkyl-Het₁-C(O)—*, *—C(O)-Het₁-C(O)—*, *—C(O)-phenyl-C₁₋₄ alkyl-NHC(O)—*, *—C(O)—C₁₋₆ alkyl-NR^{D4}*, *—NR^{D4}-cycloalkyl-**, *—O-Het₁-*, or *—NR^{D4}—C₁₋₄alkyl-Het₁-*; wherein *- represents a bond to the linker L, and *- represents a bond to G¹; Het₁ is 4- to 7-membered monocyclic heterocycle or 7- to 11-membered bicyclic heterocycle, each of which is optionally substituted with one or more (e.g., 1 to 6, 1 to 3, or 1, 2, 3, 4, 5 or 6) R^{D5}; R^{D1} is selected from H, C₁₋₆ alkyl, or halogen; R^{D2} is H or C₁₋₃ alkyl; R^{D3} is, for each occurrence, independently selected from H, halogen, C₁₋₄ alkyl, and C₁₋₄haloalkyl; R^{D4} is H or C₁₋₃ alkyl; and R^{D5} is, for each occurrence, independently selected from H, halogen, hydroxyl, C₁₋₄ alkyl, C₁₋₄haloalkyl, and C₁₋₄ alkoxy; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, or twenty-sixth embodiment.

In a twenty-eighth embodiment of the present disclosure, for the compound of formula (A), or a pharmaceutically acceptable salt thereof, DSM is a degradation signaling moiety of formula (D):

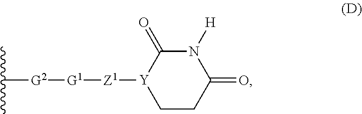

wherein

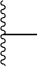

represents a bond to the linker L; Y is CR^{D1} or N; Z¹ is selected from bond, —NR^{D2}—, —O— and —CH₂—; G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more (e.g., 1 to 6, 1 to 3, or 1, 2, 3, 4, 5 or 6) R^{D3}; G² is selected from Het₁, *—NR^{D4}-Het₁-*, *—NR^{D4}-Het₁-C₁₋₄ alkyl-*, *—C₁₋₄ alkyl-C(R^{D1})=Het₁-*, *—C(O)—C₁₋₄ alkyl-Het₁-*, *-Het₁-C₁₋₆ alkyl-*, *-Het₁-O—, *—C(O)—C₁₋₄ alkyl-Het₁-C(O)—*, *—C(O)-Het₁-C(O)—*, *—C(O)-phenyl-C₁₋₄ alkyl-NHC(O)—*; wherein *- represents a bond to the linker L, and *- represents a bond to G¹; Het₁ is 4- to 7-membered monocyclic heterocycle or 7- to 11-membered bicyclic heterocycle, each of which is optionally substituted with one or more (e.g., 1 to 6, 1 to 3, or 1, 2, 3, 4, 5 or 6) R^{D5}; R^{D1} is selected from H, C₁₋₆ alkyl or halogen; R^{D2} is H or C₁₋₃ alkyl; R^{D3} is, for each occurrence, independently selected from H, halogen, C₁₋₄ alkyl, and C₁₋₄haloalkyl; R^{D4} is H or C₁₋₃ alkyl; and R^{D5} is, for each occurrence, independently selected from H, halogen, hydroxyl, C₁₋₄ alkyl, C₁₋₄haloalkyl, and C₁₋₄ alkoxy; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, or twenty-sixth embodiment.

In a twenty-ninth embodiment of the present disclosure, for the compound of formula (A), or a pharmaceutically acceptable salt thereof, DSM is a degradation signaling moiety of formula (D), wherein Het₁ is a 4 to 7 membered monocyclic saturated heterocycle containing 1 or 2 nitrogen atoms or a 7 to 8 membered saturated spiro bicyclic heterocycle containing 1 or 2 nitrogen atoms, each of which is optionally substituted with 1 or 2 R^{D5}; and the definitions for the other variables are as defined in the twenty-seventh or twenty-eighth embodiment.

In a thirtieth embodiment of the present disclosure, for the compound of formula (A), or a pharmaceutically acceptable salt thereof, DSM is a degradation signaling moiety of formula (D), wherein Het₁ is piperidine, piperazine, 1,4-diazepane, morpholine, 2-azaspiro[3.3]heptane, 2,5-diazaspiro[3.4]octane, 2,7-diazaspiro[3.5]nonane, or 2,6-diazaspiro[3.3]heptane, each of which is optionally substituted with 1 or 2 R^{D5}; and the definitions for the other variables are as defined in the twenty-seventh or twenty-eighth embodiment.

In a thirty-first embodiment of the present disclosure, for the compound of formula (A), or a pharmaceutically acceptable salt thereof, DSM is a degradation signaling moiety of formula (D) wherein Het₁ is piperidine, piperazine, 2-azaspiro[3.3]heptane, or 2,6-diazaspiro[3.3]heptane, each of which is optionally substituted with 1 or 2 R^{D5}; and the definitions for the other variables are as defined in the twenty-seventh or twenty-eighth embodiment.

In a thirty-second embodiment of the present disclosure, for the compound of formula (A), or a pharmaceutically acceptable salt thereof, DSM is a degradation signaling moiety of formula (D), wherein Het₁ is represented by any one of the following formulae:

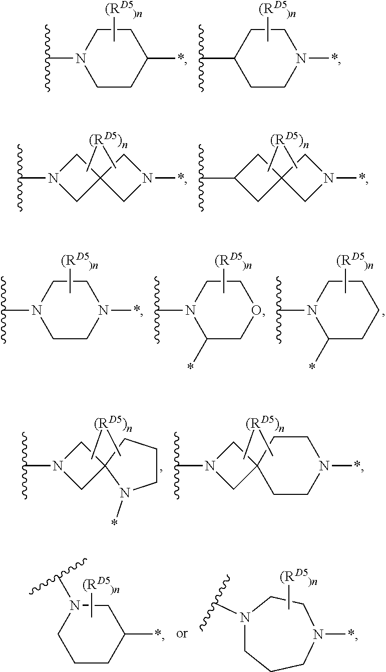

wherein n is 0, 1 or 2,

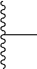

represents a bond directly or indirectly to the linker L, and -* represents directly or indirectly to G and the definitions for the other variables are as defined in the thirtieth embodiment.

In a thirty-third embodiment of the present disclosure, for the compound of formula (A), or a pharmaceutically acceptable salt thereof, DSM is a degradation signaling moiety of formula (D-I), (D-II), (D-III), (D-IV), or (D-V):

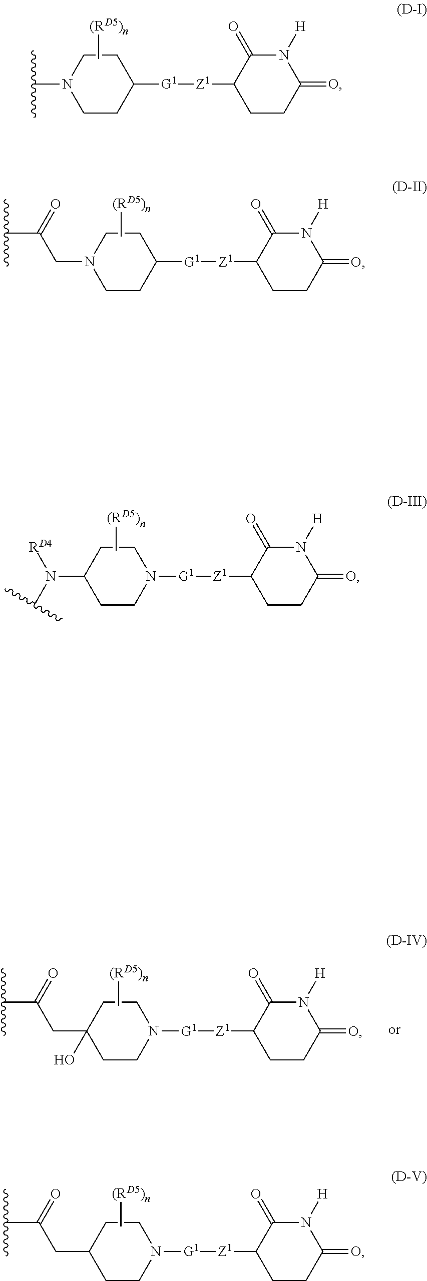

wherein

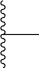

represents a bond to the linker L; Z¹ is selected from bond, —NR^{D2}— and —O—; G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more (e.g., 1 to 6, 1 to 3, or 1, 2, 3, 4, 5 or 6) R^{D3}; R^{D2} is C₁₋₃ alkyl; R^{D3} is, for each occurrence, independently selected from H, halogen and C₁₋₄ alkyl; R^{D4} is C₁₋₃ alkyl; R^{D5} is halogen; and n is 0, 1 or 2; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, or thirty-second embodiment.

In a thirty-fourth embodiment of the present disclosure, for the compound of formula (A), or a pharmaceutically acceptable salt thereof, DSM is a degradation signaling moiety of formula (D-I), (D-II), (D-III) or (D-IV):

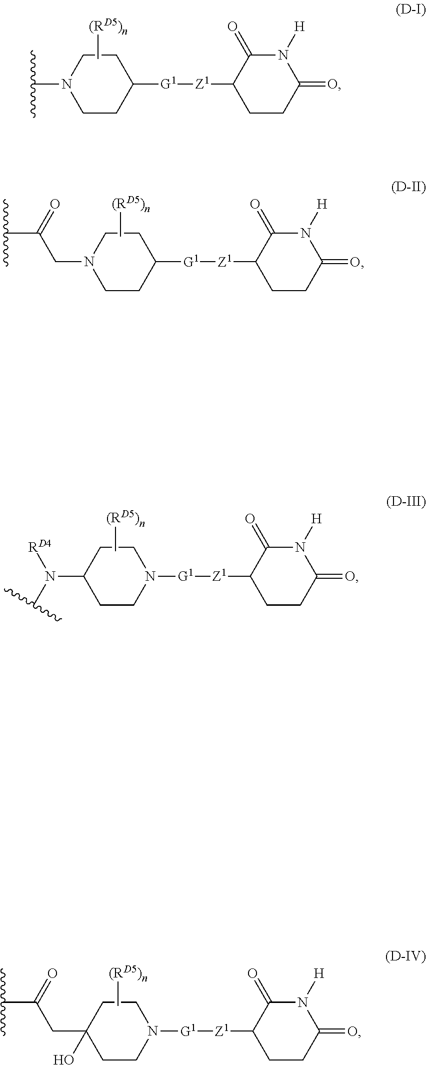

wherein

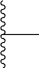

represents a bond to the linker L; Z¹ is selected from bond, —NR^{D2}— and —O—; G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more (e.g., 1 to 6, 1 to 3, or 1, 2, 3, 4, 5 or 6) R^{D3}; R^{D2} is C₁₋₃ alkyl; R^{D3} is, for each occurrence, independently selected from H, halogen and C₁₋₄ alkyl; R^{D4} is C₁₋₃ alkyl; R^{D5} is halogen; and n is 0, 1 or 2; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, or thirty-second embodiment.

In a thirty-fifth embodiment of the present disclosure, for the compound of formula (A), or a pharmaceutically acceptable salt thereof, DSM is a degradation signaling moiety of formula (D), (D-I), (D-II), (D-III), (D-IV), or (D-V), wherein G¹ is selected from phenyl, pyrazolyl, pyridinyl, pyrimidinyl, 1,3-dihydro-2H-benzo[d]imidazol-2-one, benzo[d]oxazol-2(3H)-one, 7,9-dihydro-8H-purin-8-one, 1,3-dihydro-2H-imidazo[4,5-b]pyridin-2-one, pyrazinyl, indazolyl, and indolyl, each of which is optionally substituted with 1 or 2 R^{D3}; and the definitions for the other variables are as defined in the twenty-seventh, twenty-eighth, twenty-ninth, thirtieth, thirty-first, thirty-second, thirty-third, or thirty-fourth embodiment.

In a thirty-sixth embodiment of the present disclosure, for the compound of formula (A), or a pharmaceutically acceptable salt thereof, DSM is a degradation signaling moiety of formula (D), (D-I), (D-II), (D-III), (D-IV), or (D-V) wherein G¹ is selected from phenyl, pyrazolyl, pyridinyl and pyrimidinyl, 1,3-dihydro-2H-benzo[d]imidazol-2-one, indazolyl, and indolyl, each of which is optionally substituted with 1 or 2 R^{D3}; and the definitions for the other variables are as defined in the twenty-seventh, twenty-eighth, twenty-ninth, thirtieth, thirty-first, thirty-second, thirty-third, or thirty-fourth embodiment.

In a thirty-seventh embodiment of the present disclosure, for the compound of formula (A), or a pharmaceutically acceptable salt thereof, DSM is a degradation signaling moiety of formula (D), (D-I), (D-II), (D-III), (D-IV), or (D-V), wherein G¹ is represented by any one of the following formulae:

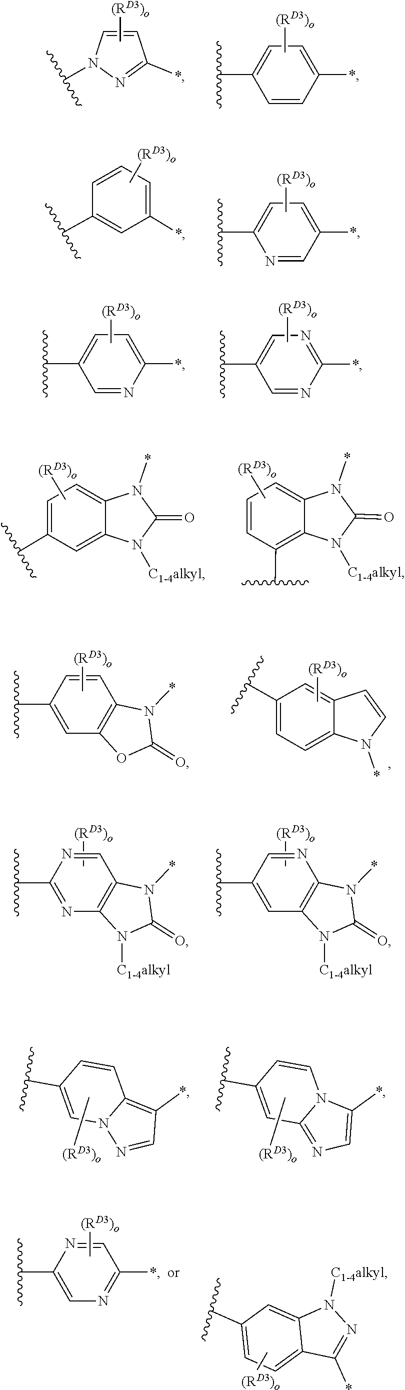

wherein o is 0, 1 or 2,

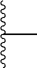

represents a bond to G², and -* represents a bond to Z¹; and the definitions for the other variables are as defined in the twenty-seventh, twenty-eighth, twenty-ninth, thirtieth, thirty-first, thirty-second, thirty-third, or thirty-fourth embodiment.

In a thirty-eighth embodiment of the present disclosure, for the compound of formula (A), or a pharmaceutically acceptable salt thereof, DSM is a degradation signaling moiety of formula (D), (D-I), (D-II), (D-III), (D-IV), or (D-V), wherein G¹ is 6- to 10-membered aryl or 5- to 10-membered heteroaryl; wherein the 6- to 10-membered aryl and 5- to 10-membered heteroaryl represented by G¹ are each optionally substituted with 1 or 2 R^{D3}; and the definitions for the other variables are as defined in the twenty-seventh, twenty-eighth, twenty-ninth, thirtieth, thirty-first, thirty-second, thirty-third, or thirty-fourth embodiment.

In a thirty-ninth embodiment of the present disclosure, for the compound of formula (A), or a pharmaceutically acceptable salt thereof, DSM is a degradation signaling moiety of formula (D), (D-I), (D-II), (D-III), (D-IV), or (D-V), wherein G¹ is represented by any one of the following formulae:

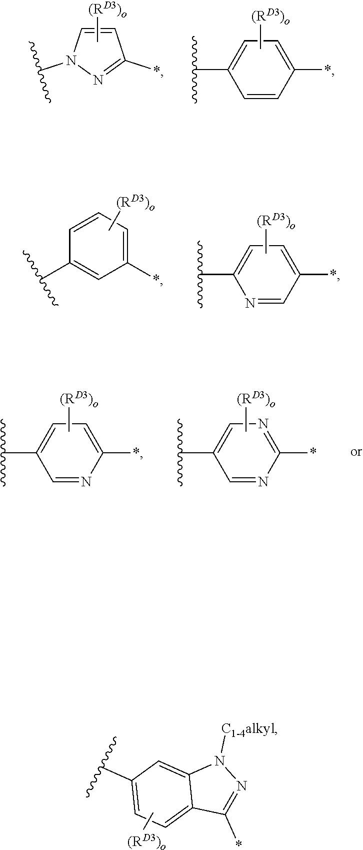

wherein o is 0, 1 or 2,

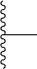

represents a bond to G², and -* represents a bond to Z¹; and the definitions for the other variables are as defined in the twenty-seventh, twenty-eighth, twenty-ninth, thirtieth, thirty-first, thirty-second, thirty-third, or thirty-fourth embodiment.

In a fortieth embodiment of the present disclosure, for the compound of formula (A), or a pharmaceutically acceptable salt thereof, DSM is a degradation signaling moiety of formula (D), (D-I), (D-II), (D-III), (D-IV), or (D-V) wherein R^{D1} is H, —CH₃ or F; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, or thirty-ninth embodiment.

In a forty-first embodiment of the present disclosure, for the compound of formula (A), or a pharmaceutically acceptable salt thereof, DSM is a degradation signaling moiety of formula (D), (D-I), (D-II), (D-III), (D-IV), or (D-V), wherein R^{D2} is H; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, or fortieth embodiment.

In a forty-second embodiment of the present disclosure, for the compound of formula (A), or a pharmaceutically acceptable salt thereof, DSM is a degradation signaling moiety of formula (D), (D-I), (D-II), (D-III), (D-IV), or (D-V) wherein R^{D3} is, for each occurrence, independently selected from H, Cl, F and —CH₃; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, or forty-first embodiment.

In a forty-third embodiment of the present disclosure, for the compound of formula (A), or a pharmaceutically acceptable salt thereof, DSM is a degradation signaling moiety of formula (D), (D-I), (D-II), (D-III), (D-IV), (D-V), wherein R^{D4} is —CH₃; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, or forty-second embodiment.

In a forty-fourth embodiment of the present disclosure, for the compound of formula (A), or a pharmaceutically acceptable salt thereof, DSM is a degradation signaling moiety of formula (D), (D-I), (D-II), (D-III), (D-IV), (D-V) wherein R^{D5} for each occurrence, is independently F or OH; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, or forty-third embodiment. In a forty-fifth embodiment of the present disclosure, for the compound of formula (A), or a pharmaceutically acceptable salt thereof, DSM represents any one of the following attached to L:

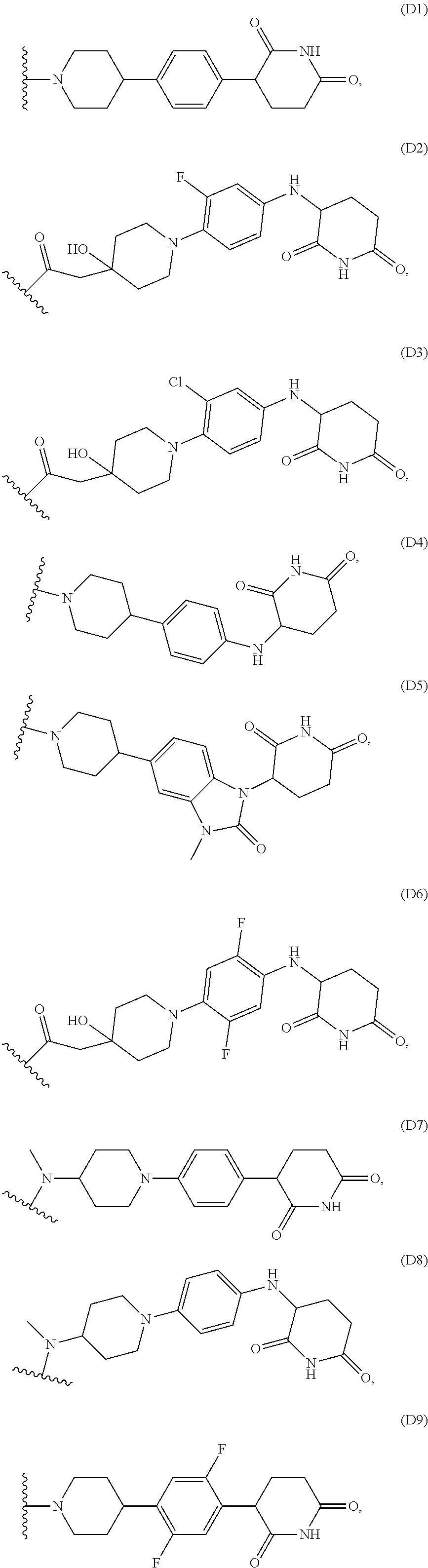

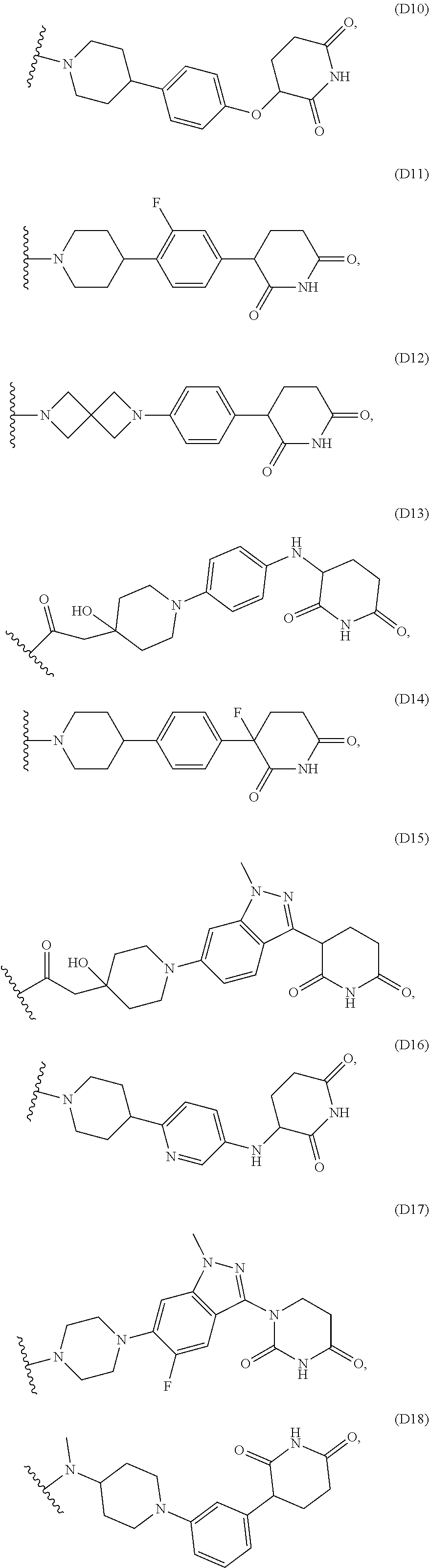

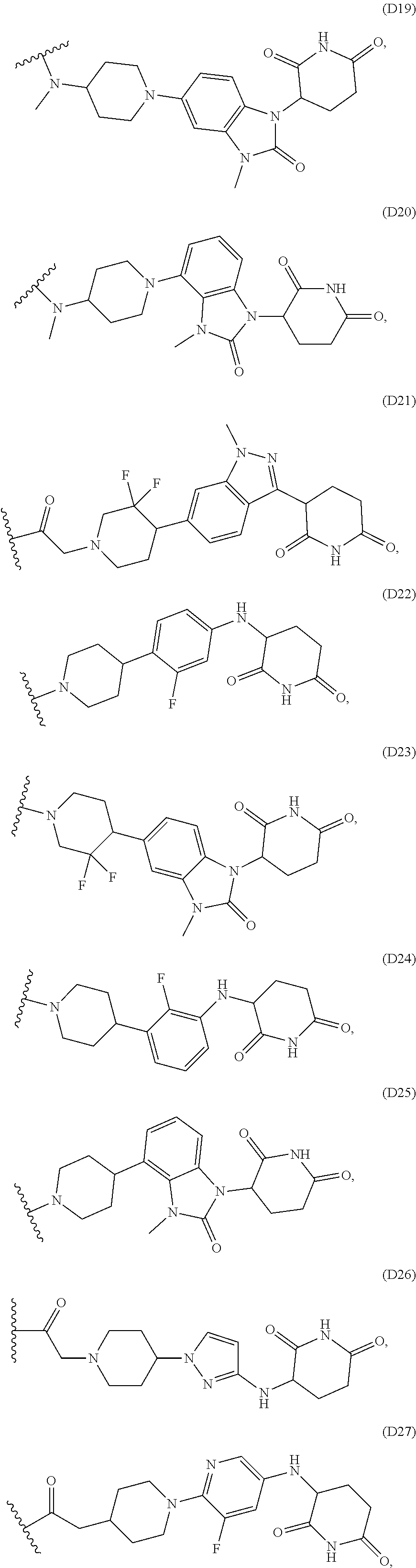

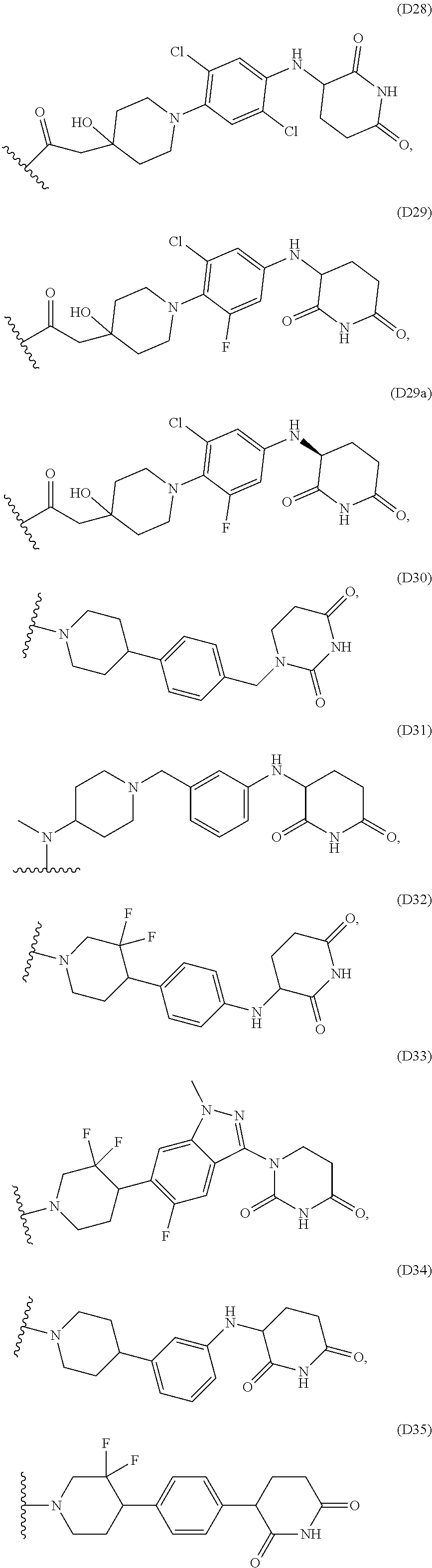

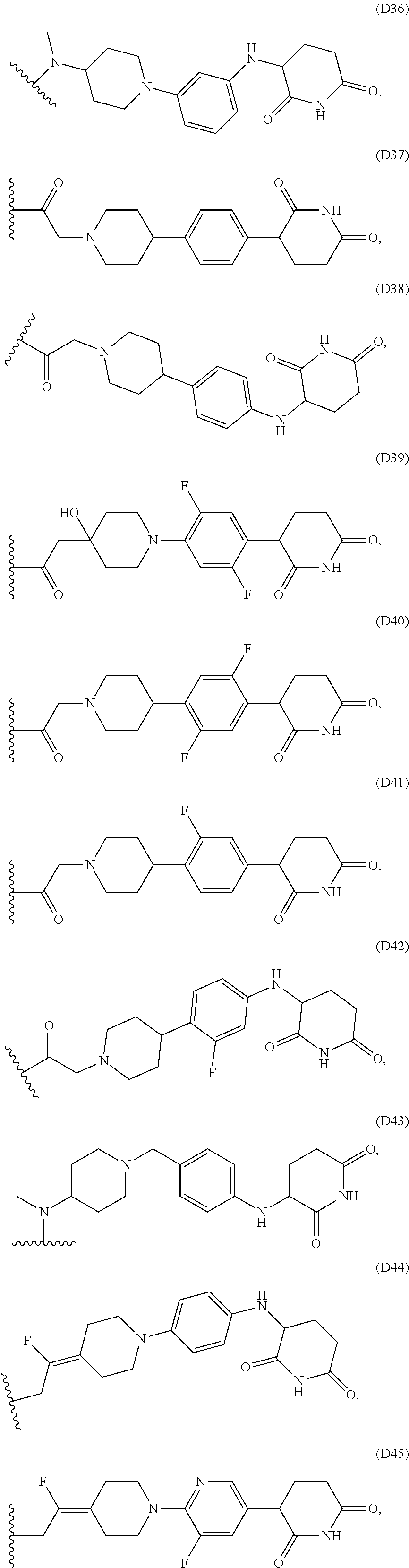

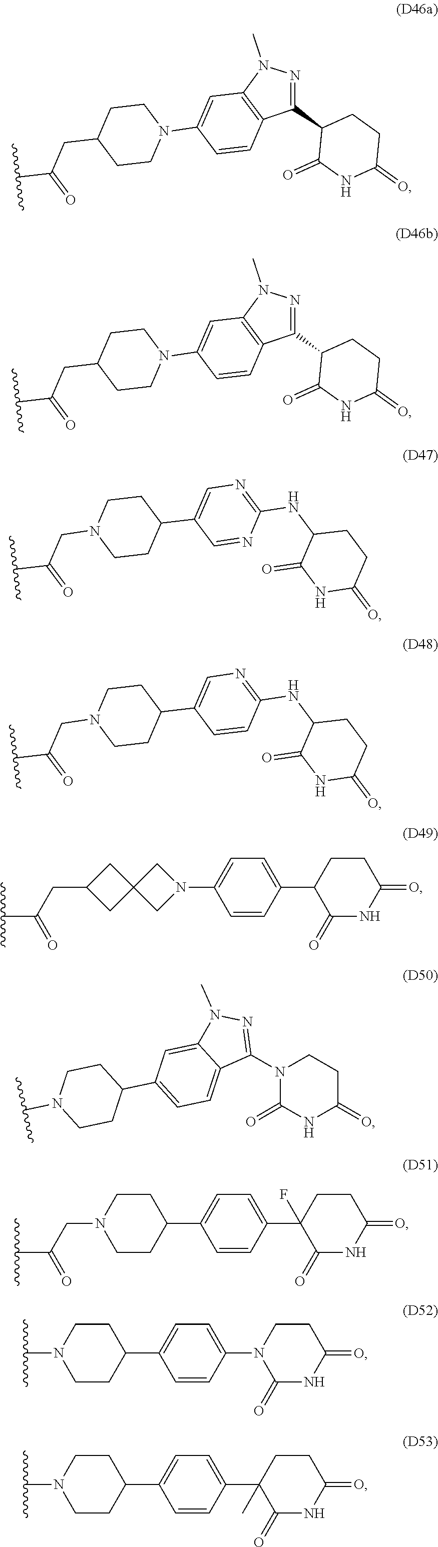

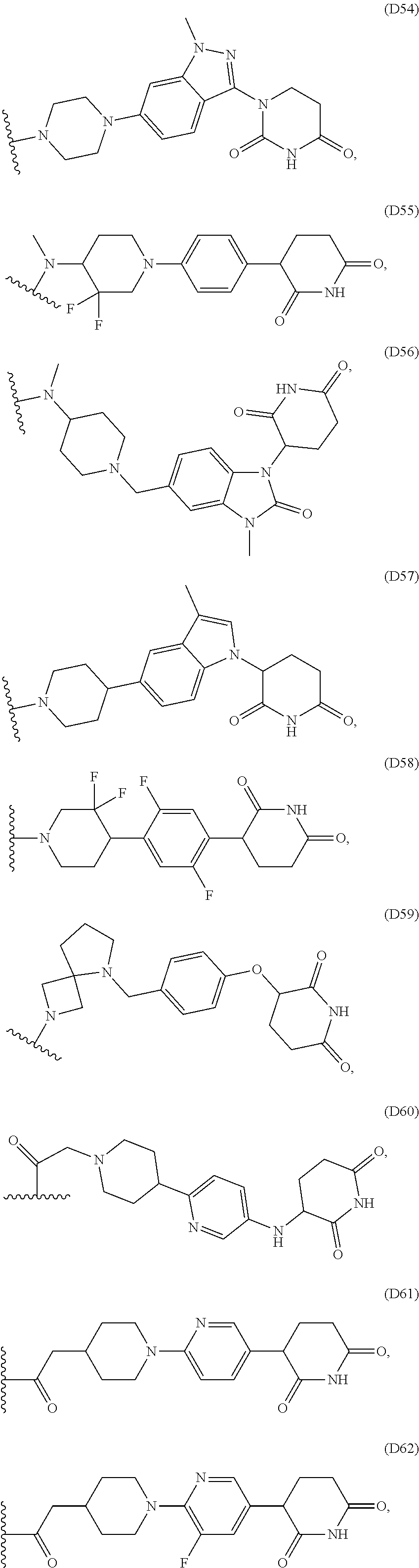

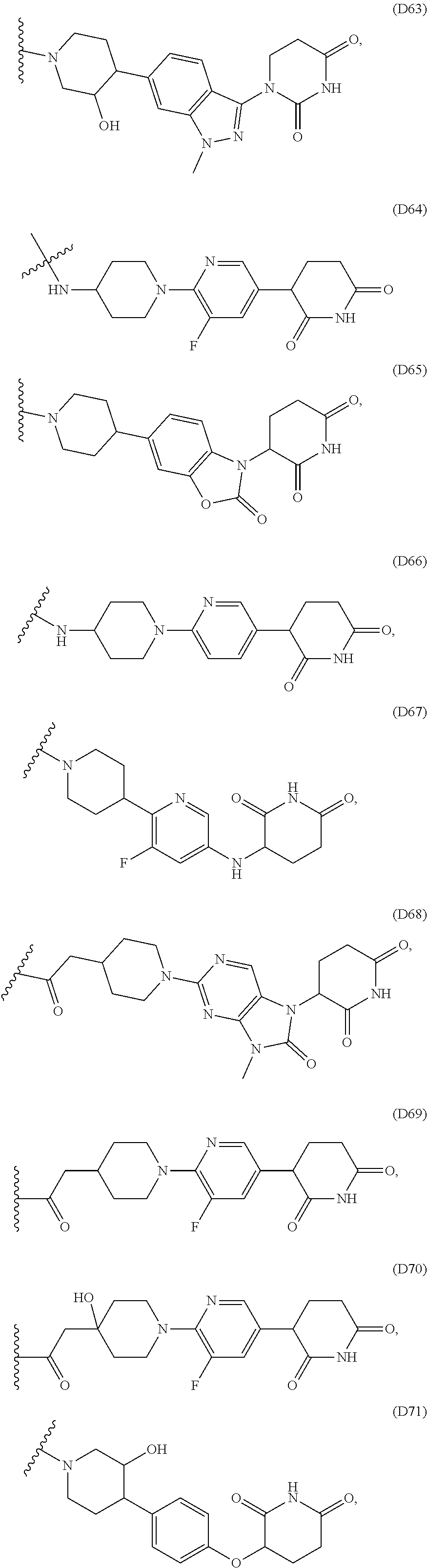

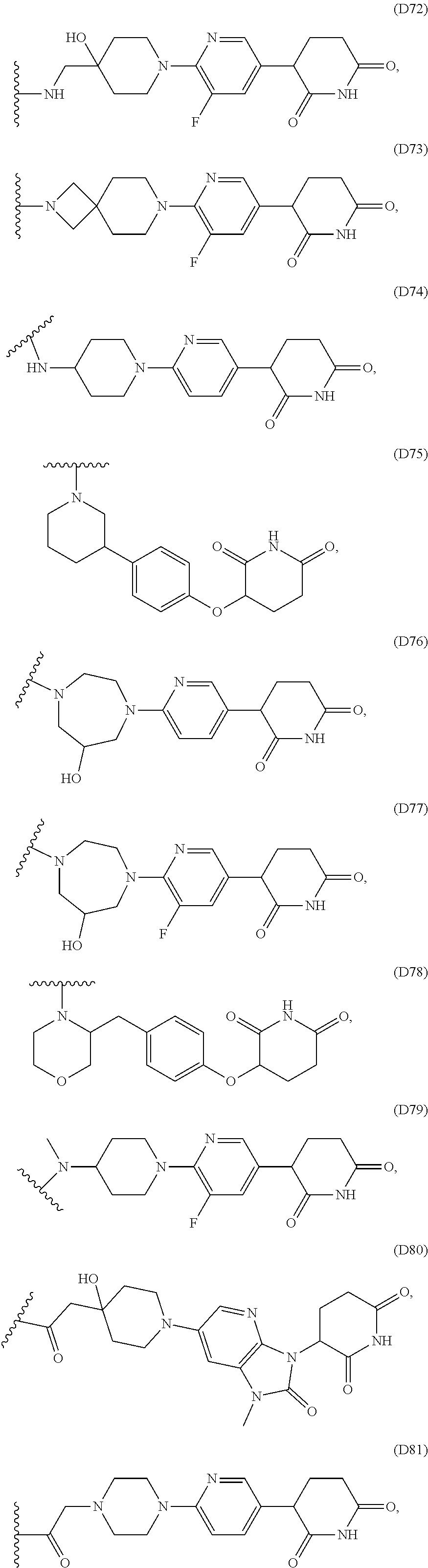

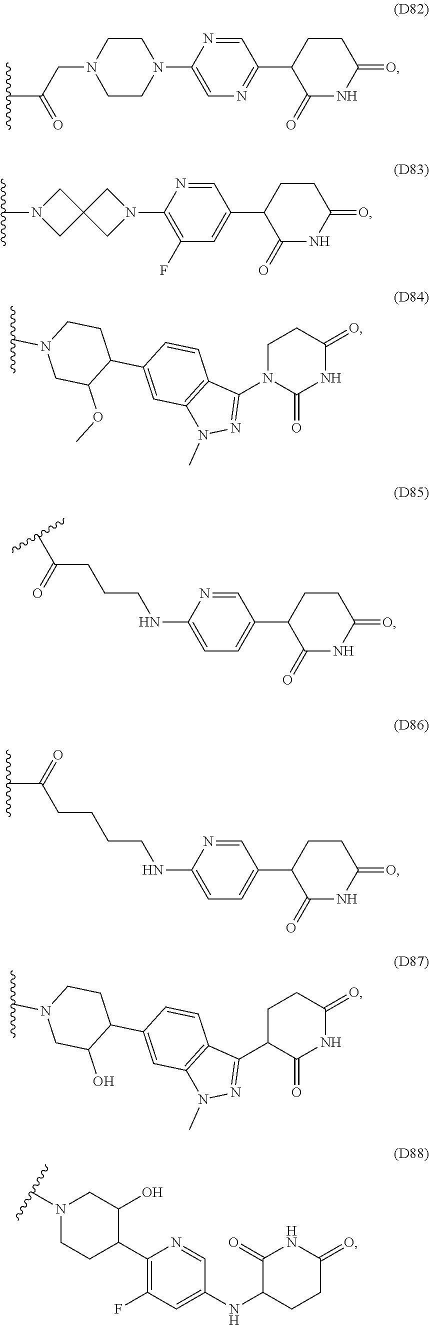

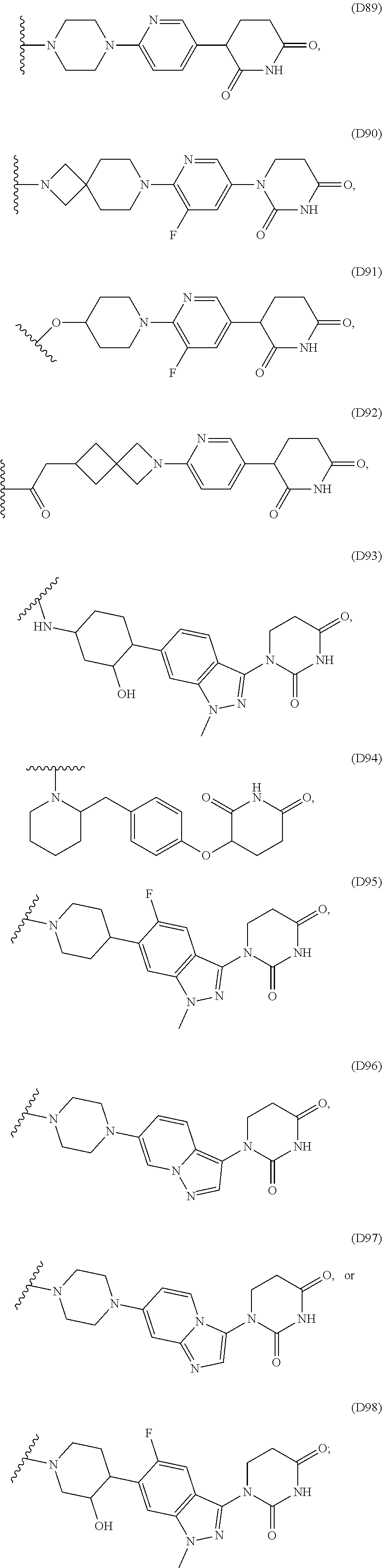

and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, or twenty-sixth embodiment.

### C. Linker

In a forty-sixth embodiment of the present disclosure, for the compound of formula (A), or a pharmaceutically acceptable salt thereof, L is a bond, C₁₋₈ alkyl or is represented by formula (L-1), (L-2) or (L-3):

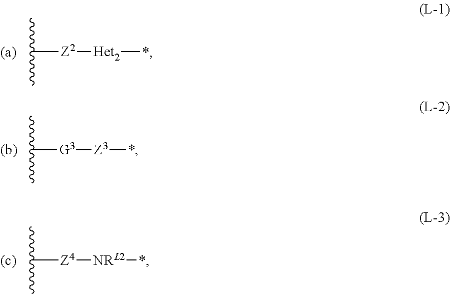

wherein Z² is bond or C₁₋₄ alkyl optionally substituted with one or more (e.g., 1 to 6, 1 to 3, or 1, 2, 3, 4, 5 or 6) halogen; Het₂ is 4- to 7-membered heterocycle optionally substituted by one or more (e.g., 1 to 6, 1 to 3, or 1, 2, 3, 4, 5 or 6) R^{L1}; G³ is C₃₋₇ cycloalkyl or 4- to 7-membered heterocycle; wherein the C₃₋₇ cycloalkyl and 4- to 7-membered heterocycle represented by G³ are each optionally substituted with one or more (e.g., 1 to 6, 1 to 3, or 1, 2, 3, 4, 5 or 6) R^{L3}; Z³ is C₁₋₄ alkyl, —C(O)—, or *—C₁₋₄ alkyl-C(O)—*, wherein *- represents a bond connected to G³; -* is a bond connected to the DSM; and the C₁₋₄ alkyl is optionally substituted with one or more halogen; Z⁴ is C₁₋₄ alkyl optionally substituted by R^{L4}; R^{L1} is, for each occurrence, independently selected from H, halogen, C₁₋₄ alkyl and C₁₋₄haloalkyl; R^{L2} is H or C₁₋₄ alkyl; R^{L3} is, for each occurrence, independently selected from H, halogen, C₁₋₄ alkyl and C₁₋₄haloalkyl; R^{L4} is halo, —OR^{L5}, or C₁₋₄ alkyl optionally substituted by halogen, C₃₋₇ cycloalkyl, phenyl, 4- to 7-membered monocyclic saturated heterocycle, or 5- to 6-membered heteroaryl, wherein the C₃₋₇ cycloalkyl, phenyl, 4- to 7-membered monocyclic saturated heterocycle, and 5- to 6-membered heteroaryl are each optionally substituted with one to three substituents independently selected from halogen, C₁₋₄ alkyl, C₁₋₄ haloalkyl, C₁₋₄ alkoxy and C₁₋₄ haloalkoxy; R^{L5} is H, C₁₋₄ alkyl or C₁₋₄ haloalkyl;

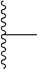

represents a bond to the IRAK binding moiety; and -* represents a bond to the degradation signaling moiety DSM; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, or forty-fifth embodiment.

In a forty-seventh embodiment of the present disclosure, for the compound of formula (A), or a pharmaceutically acceptable salt thereof, L is a bond, C₁₋₈ alkyl or is represented by formula (L-1), (L-2) or (L-3):

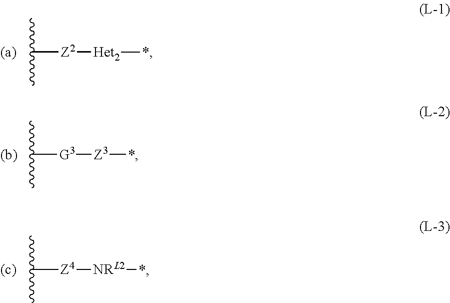

wherein Z² is bond or C₁₋₄ alkyl optionally substituted with one or more (e.g., 1 to 6, 1 to 3, or 1, 2, 3, 4, 5 or 6) halogen; Het₂ is 4- to 7-membered heterocycle optionally substituted by one or more (e.g., 1 to 6, 1 to 3, or 1, 2, 3, 4, 5 or 6) R^{L1}; G³ is C₃₋₇ cycloalkyl or 4- to 7-membered heterocycle; wherein the C₃₋₇ cycloalkyl and 4- to 7-membered heterocycle represented by G³ are each optionally substituted with one or more (e.g., 1 to 6, 1 to 3, or 1, 2, 3, 4, 5 or 6) R^{L3}; Z³ is C₁₋₄ alkyl or *—C₁₋₄ alkyl-C(O)—*, wherein *- represents a bond connected to G³; -* is a bond connected to the DSM; and the C₁₋₄ alkyl is optionally substituted with one or more halogen; Z⁴ is C₁₋₄ alkyl optionally substituted by R^{L4}; R^{L1} is, for each occurrence, independently selected from H, halogen, C₁₋₄ alkyl and C₁₋₄haloalkyl; R^{L2} is H or C₁₋₄ alkyl; R^{L3} is, for each occurrence, independently selected from H, halogen, C₁₋₄ alkyl and C₁₋₄haloalkyl; R^{L4} is halo, —OR^{L5}, or C₁₋₄ alkyl optionally substituted by halogen, C₃₋₇ cycloalkyl, phenyl, 4- to 7-membered monocyclic saturated heterocycle, or 5- to 6-membered heteroaryl, wherein the C₃₋₇ cycloalkyl, phenyl, 4- to 7-membered monocyclic saturated heterocycle, and 5- to 6-membered heteroaryl are each optionally substituted with one to three substituents independently selected from halogen, C₁₋₄ alkyl, C₁₋₄ haloalkyl, C₁₋₄ alkoxy and C₁₋₄ haloalkoxy; R^{L5} is H, C₁₋₄ alkyl or C₁₋₄ haloalkyl;

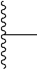

represents a bond to the IRAK binding moiety; and -* represents a bond to the degradation signaling moiety DSM; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, or forty-fifth embodiment.

In a forty-eighth embodiment of the present disclosure, for the compound of formula (A), or a pharmaceutically acceptable salt thereof, L is a bond or is represented by formula (L-1), (L-2) or (L-3), wherein Z² is bond or —CH₂—; Het₂ is selected from azetidinyl, piperidinyl and pyrrolidinyl; wherein the azetidinyl, piperidinyl and pyrrolidinyl represented by Het₂ are each optionally substituted by one or more (e.g., 1 to 6, 1 to 3, or 1, 2, 3, 4, 5 or 6) R^{L1}; G³ is cyclohexyl or piperidinyl; wherein the cyclohexyl and piperidinyl represented by G³ are each optionally substituted with one or more (e.g., 1 to 6, 1 to 3, or 1, 2, 3, 4, 5 or 6) R^{L3}; Z³ is —CH₂— or *—CH₂—C(O)—*; and Z⁴ is —CH₂— optionally substituted by R^{L4}; and the definitions for the other variables are as defined in the forty-sixth or forty-seventh embodiment.

In a forty-ninth embodiment of the present disclosure, for the compound of formula (A), or a pharmaceutically acceptable salt thereof, L is a bond or is represented by formula (L-1), (L-2) or (L-3), wherein R^{L1} is H; R^{L2} is H; R^{L3} is H; and R^{L4} is benzyl; and the definitions for the other variables are as defined in the forty-sixth, forty-seventh, or forty-eighth embodiment.

In a fiftieth embodiment of the present disclosure, for the compound of formula (A), or a pharmaceutically acceptable salt thereof, L is represented by formula (L-1) and Het₂ is represented by one of the formulae:

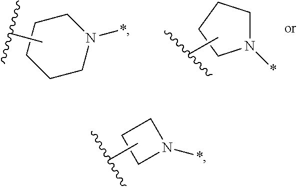

wherein

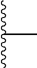

represents a bond to Z²; and -* represents a bond to the degradation signaling moiety DSM; and the definitions for the other variables are as defined in the forty-sixth, forty-seventh, or forty-eighth embodiment.

In a fifty-first embodiment of the present disclosure, for the compound of formula (A), or a pharmaceutically acceptable salt thereof, L is represented by formula (L-2) and G³ is represented by one of the formulae:

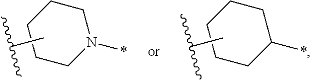

wherein

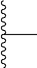

represents a bond to the IRAK binding moiety; and -* represents a bond to Z³; and the definitions for the other variables are as defined in the forty-sixth, forty-seventh, or forty-eighth embodiment.

In a fifty-second embodiment of the present disclosure, for the compound of formula (A), or a pharmaceutically acceptable salt thereof, L is represented by formula (L-1) and Het₂ is:

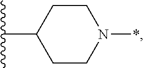

wherein

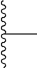

represents a bond to Z²; and -* represents a bond to the degradation signaling moiety DSM; and the definitions for the other variables are as defined in the forty-sixth, forty-seventh, or forty-eighth embodiment.

In a fifty-third embodiment of the present disclosure, for the compound of formula (A), or a pharmaceutically acceptable salt thereof, L is represented by formula (L-2) and G³ is represented by:

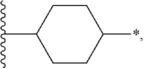

wherein

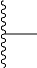

represents a bond to the IRAK binding moiety; and -* represents a bond to Z³; and the definitions for the other variables are as defined in the forty-sixth, forty-seventh, or forty-eighth embodiment.

In a fifty-fourth embodiment of the present disclosure, for the compound of formula (A), or a pharmaceutically acceptable salt thereof, L is represented by any one of the following formulae:

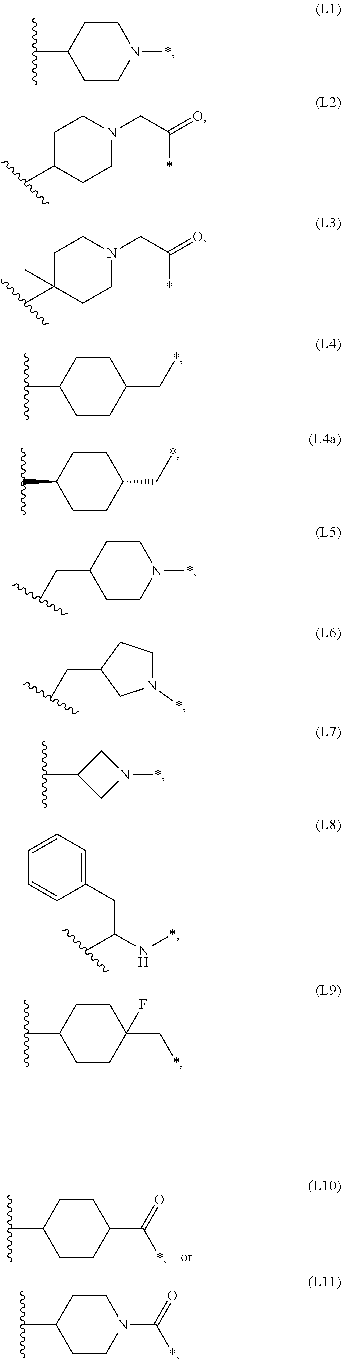

wherein

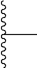

represents a bond to the IRAK binding moiety; and -* represents a bond to the degradation signaling moiety DSM; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, or forty-fifth embodiment.

In a fifty-fifth embodiment, for the compound of formula (A), the compound is represented by one of the following formulae:

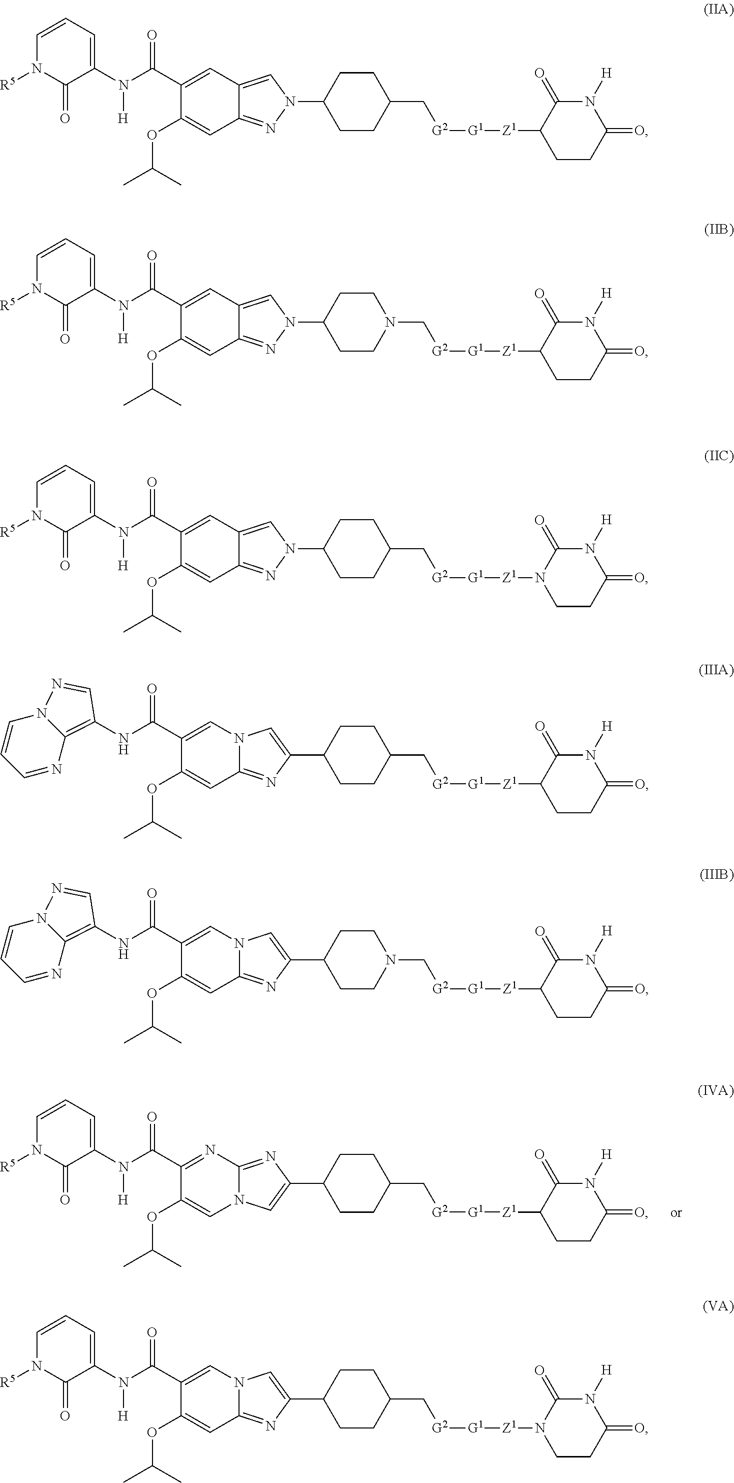

or a pharmaceutically acceptable salt thereof, wherein Z¹ is a bond or —O—; G¹ is phenyl, 6-membered heteroaryl or 9-membered partially saturated bicyclic heterocycle, each of which is optionally substituted with 1 or 2 substituents independently selected from halo and C₁₋₂alkyl; G² is Het₁, *—NR^{D4}-Het₁-*, or *—C(O)—C₁₋₂ alkyl-Het₁-*; wherein *- represents a bond to the linker L, and *- represents a bond to G¹; Het₁ is piperidine optionally substituted with 1 or 2 halo or OH; R⁵ is C₃₋₄cycloalkyl is optionally substituted with 1 halo; R^{D4} is H or C₁₋₂alkyl; and the remaining variables are as described in the first embodiment.

In a fifty-sixth embodiment, for the compound of formula (A), (IIA), (IIB), (IIC), (IIIA), (IIIB), (IVA), or (VA), or a pharmaceutically acceptable salt thereof, wherein G¹ is phenyl, pyridinyl, indazoyl, or 1,3-dihydro-2H-benzo[d]imidazol-2-one, each of which is optionally substituted with 1 or 2 substituents independently selected from halo and C₁₋₂alkyl; G² is Het₁, *—NH-Het₁-*, or *—C(O)—CH₂—Het₁-*; wherein *- represents a bond to the linker L, and *- represents a bond to G¹; Het₁ is piperidine optionally substituted with 1 or 2 halo or OH; and the remaining variables are as described in the fifty-fifth embodiment.

In a fifty-seventh embodiment, for the compound of formula (A), (IIA), (IIB), (IIC), (IIIA), (IIIB), (IVA), or (VA), or a pharmaceutically acceptable salt thereof, wherein G¹ is

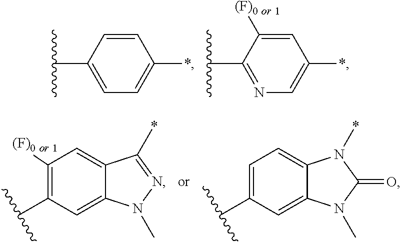

wherein

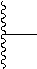

represents a bond to G², and -* represents a bond to Z¹;
Het₁ is

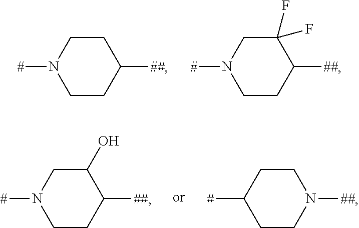

wherein #- represents a bond to the linker, —NH—, or —C(O)—CH₂— and ##- represents a bond to G¹;
R⁵ is cyclopropyl or

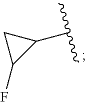 [SMILES: *C1CC1F]

and the remaining variables are as described in the fifty-sixth embodiment.

In a fifty-eighth embodiment of the present disclosure, the compound of formula (A), or a pharmaceutically acceptable salt thereof, is a compound of any one of Examples 1 to 199 or a pharmaceutically acceptable salt thereof.

In one embodiment of the present disclosure, the compound of formula (A) is not a compound of the following formula:

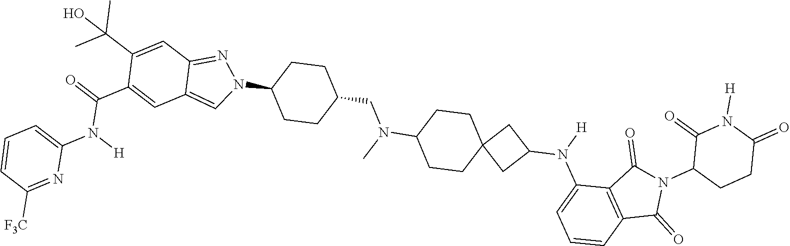 [SMILES: CN(C[C@H]1CC[C@H](n2cc3cc(C(=O)Nc4cccc(C(F)(F)F)n4)c(C(C)(C)O)cc3n2)CC1)C1CCC2(CC1)CC(Nc1cccc3c1C(=O)N(C1CCC(=O)NC1=O)C3=O)C2]

or a pharmaceutically acceptable salt thereof.

### III. Pharmaceutical Composition and Methods of Uses

Another aspect of the present disclosure is a pharmaceutical composition comprising at least one compound described herein (e.g., a compound or a pharmaceutically acceptable salt thereof described in any of the embodiments described above), and at least one pharmaceutically acceptable carrier.

The compounds of the present disclosure are typically used as a pharmaceutical composition (e.g., a compound of the present disclosure and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present disclosure and a solvent (i.e., solvate) or water (i.e., hydrate).

Compounds of the present disclosure have been found to modulate IRAK4 activity and may be beneficial for the treatment of neurological, neurodegenerative and other additional diseases

In some embodiments, the compounds described herein (e.g., a compound or a pharmaceutically acceptable salt thereof described in any of the embodiments described above) can be used to cause the degradation of IRAK4 proteins. In some embodiments, the compounds described herein (e.g., a compound or a pharmaceutically acceptable salt thereof described in any of the embodiments described above) can be used to modulate (e.g., decrease) the level of IRAK4 proteins. In some embodiments, the compounds or pharmaceutically acceptable salts thereof described herein (e.g., a compound or a pharmaceutically acceptable salt thereof described in any of the embodiments described above) can be used to modulate (e.g., decrease) the activity of IRAK4, or to otherwise affect the properties and/or behavior of IRAK4, e.g., stability, phosphorylation, kinase activity, interactions with other proteins, etc.

In some embodiments, the present disclosure provides methods of decreasing protein levels of IRAK4 and/or IRAK4 enzymatic activity. In some embodiments, such methods include contacting a cell with an effective amount of a compound described herein (e.g., a compound or a pharmaceutically acceptable salt thereof described in any of the embodiments described above).

One aspect of the present disclosure includes a method of treating a disorder responsive to degradation of IRAK4 and/or inhibition of IRAK4 activity in a subject comprising administering to the subject an effective amount of at least one compound described herein (e.g., a compound or a pharmaceutically acceptable salt thereof described in any of the embodiments described above), or a pharmaceutical composition described herein.

One embodiment of the disclosure includes a method for treating an autoimmune disease, cancer, cardiovascular disease, a disease of the central nervous system, a disease of the skin, an ophthalmic disease and condition, and bone disease in a subject, the method comprising administering to the patient a therapeutically effective amount of a compound disclosed herein, or a pharmaceutically acceptable salt thereof, thereby treating the autoimmune disease, cancer, cardiovascular disease, disease of the central nervous system, disease of the skin, ophthalmic disease and condition, and bone disease in the subject.

In one embodiment, the cardiovascular disease is selected from stroke and atherosclerosis. In one embodiment, the disease of the central nervous system is a neurodegenerative disease. In one embodiment, the disease of the skin is selected from rash, contact dermatitis, psoriasis, Hidradenitis Suppurativa and atopic dermatitis. In one embodiment, the bone disease is selected from osteoporosis and osteoarthritis.

In one embodiment, the present disclosure provides methods of treating autoimmune disorders, inflammatory disorders, and cancers in a subject in need thereof comprising administering to the subject an effective amount of at least one compound described herein (e.g., a compound or a pharmaceutically acceptable salt thereof described in any of the embodiments described above), or a pharmaceutical composition described herein.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barre syndrome (GBS), Hashimoto's disease, idiopathic thrombocytopenic purpura, lupus erythematosus, Cutaneous Lupus Erythematosus (CLE), Neuromyelitis optica (NMO), mixed connective tissue disease, multiple sclerosis, myasthenia gravis, pemphigus vulgaris, pernicious anaemia, polymyositis, primary biliary cirrhosis, Sjogren's syndrome, temporal arteritis, and Wegener's granulomatosis.

In one embodiment, the autoimmune disease is selected from rheumatoid arthritis, systemic lupus erythematosus, multiple sclerosis, diabetes, systemic sclerosis, and Sjogren's syndrome. In one embodiment, the autoimmune disease is type 1 diabetes.

The term “inflammatory disorders” includes diseases or disorders involving acute or chronic inflammation such as allergies, asthma, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis. In some embodiments, the present disclosure provides a method of treating rheumatoid arthritis or lupus. In some embodiments, the present disclosure provides a method of treating multiple sclerosis. In some embodiments, the present disclosure provides a method of treating systemic lupus erythematosus or atopic dermatitis.

One embodiment of the disclosure includes a method for treating an inflammatory disease in a subject, the method comprising administering to the patient a therapeutically effective amount of a compound described herein, or a pharmaceutically acceptable salt thereof, thereby treating the inflammatory disease in the subject.

In one embodiment, the inflammatory disease is a pulmonary disease or a disease of the airway. In one embodiment, the pulmonary disease and disease of the airway is selected from Adult Respiratory Disease Syndrome (ARDS), Chronic Obstructive Pulmonary Disease (COPD), pulmonary fibrosis, interstitial lung disease, asthma, chronic cough, and allergic rhinitis.

In one embodiment, the inflammatory disease is selected from transplant rejection, CD14 mediated sepsis, non-CD14 mediated sepsis, inflammatory bowel disease, Behcet's syndrome, ankylosing spondylitis, sarcoidosis, and gout. In one embodiment, the inflammatory bowel disease is selected from Crohn's disease and ulcerative colitis.

One embodiment of the disclosure includes a method for treating an ischemic fibrotic disease, the method comprising administering to the patient a therapeutically effective amount of a compound described herein, or a pharmaceutically acceptable salt thereof, thereby treating the ischemic fibrotic disease in the subject. In one embodiment, the ischemic fibrotic disease is selected from stroke, acute lung injury, acute kidney injury, ischemic cardiac injury, acute liver injury, and ischemic skeletal muscle injury.

One embodiment of the disclosure includes a method for treating post-organ transplantation fibrosis, the method comprising administering to the patient a therapeutically effective amount of a compound described herein, or a pharmaceutically acceptable salt thereof, thereby treating post-organ transplantation fibrosis in the subject.

One embodiment of the disclosure includes a method for treating hypertensive or diabetic end organ disease, the method comprising administering to the patient a therapeutically effective amount of a compound described herein, or a pharmaceutically acceptable salt thereof, thereby treating hypertensive or diabetic end organ disease in the subject.

One embodiment of the disclosure includes a method for treating hypertensive kidney disease, the method comprising administering to the patient a therapeutically effective amount of a compound described herein, or a pharmaceutically acceptable salt thereof, thereby treating hypertensive kidney disease in the subject.

One embodiment of the disclosure includes a method for treating idiopathic pulmonary fibrosis (IPF), the method comprising administering to the patient a therapeutically effective amount of a compound described herein, or a pharmaceutically acceptable salt thereof, thereby treating IPF in the subject.

One embodiment of the disclosure includes a method for treating scleroderma or systemic sclerosis, the method comprising administering to the patient a therapeutically effective amount of a compound described herein, or a pharmaceutically acceptable salt thereof, thereby treating scleroderma or systemic sclerosis in the subject.

One embodiment of the disclosure includes a method for treating liver cirrhosis, the method comprising administering to the patient a therapeutically effective amount of a compound described herein, or a pharmaceutically acceptable salt thereof, thereby treating liver cirrhosis in the subject.

One embodiment of the disclosure includes a method for treating fibrotic diseases wherein tissue injury and/or inflammation are present, the method comprising administering to the patient a therapeutically effective amount of a compound described herein, or a pharmaceutically acceptable salt thereof, thereby treating fibrotic diseases where tissue injury and/or inflammation are present in the subject. The fibrotic diseases include, for example, pancreatitis, peritonitis, burns, glomerulonephritis, complications of drug toxicity, and scarring following infections.

Scarring of the internal organs is a major global health problem, which is the consequence of subclinical injury to the organ over a period of time or as the sequela of acute severe injury or inflammation. All organs may be affected by scarring and currently there are few therapies the specifically target the evolution of scarring. Increasing evidence indicates that scarring per se provokes further decline in organ function, inflammation and tissue ischemia. This may be directly due the deposition of the fibrotic matrix which impairs function such as in contractility and relaxation of the heart and vasculature or impaired inflation and deflation of lungs, or by increasing the space between microvasculature and vital cells of the organ that are deprived of nutrients and distorting normal tissue architecture. However recent studies have shown that myofibroblasts themselves are inflammatory cells, generating cytokines, chemokines and radicals that promote injury; and myofibroblasts appear as a result of a transition from cells that normally nurse and maintain the microvasculature, known as pericytes. The consequence of this transition of phenotype is an unstable microvasculature that leads to aberrant angiogenesis, or rarefaction.

The present disclosure relates to methods and compositions for treating, preventing, and/or reducing scarring in organs. More particularly, the present disclosure relates to methods and composition for treating, preventing, and/or reducing scarring in kidneys. Some non-limiting examples of organs include: kidney, hearts, lungs, stomach, liver, pancreas, hypothalamus, stomach, uterus, bladder, diaphragm, pancreas, intestines, colon, and so forth.

It is contemplated that the present disclosure, methods and compositions described herein can be used as an antifibrotic, or used to treat, prevent, and/or reduce the severity and damage from fibrosis. It is additionally contemplated that the present disclosure, methods and compositions described herein can be used to treat, prevent, and/or reduce the severity and damage from fibrosis.

The compounds of the present disclosure (e.g., a compound or a pharmaceutically acceptable salt thereof described in any of the embodiments described above) may be useful in the treatment of cancer, for example a cancer selected from solid tumor cancers and hematopoietic cancers.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer). In some embodiments, the present disclosure provides a method of treating leukemia or lymphoma.

Examples of solid tumor cancers include central nervous system cancer, brain cancer, breast cancer, head and neck cancer, lung cancer; esophageal and esophagogastric junction cancer, gastric cancer, colorectal cancer, rectal cancer, anal cancer, hepatobiliary cancer, pancreatic cancer, non-melanoma skin cancer, melanoma, renal cancer, prostate cancer, bladder cancer, uterine cancer, cervical cancer, ovarian cancer, bone cancer, neuroendocrine cancer, mesothelioma cancer, testicular cancer, thymoma and thymic carcinoma, and thyroid cancer.

Examples of hematopoietic cancers include B-cell neoplasms (including rare B-cell malignancies), Hodgkin lymphoma, non-Hodgkin lymphoma, post-transplant lymphoproliferative disorder, hairy cell leukemia, histiocytic and dendritic neoplasms.

Examples of B-cell neoplasms include chronic lymphocytic leukemia (CLL), mantle cell lymphoma (MCL), small lymphocytic lymphoma (SLL), Waldenstrom's macroglobulinemia, diffuse large B-cell lymphoma (DLBCL), follicular lymphoma, Burkitt lymphoma, Marginal Zone Lymphoma, immunoblastic large cell lymphoma, Richter Syndrome, and precursor B-lymphoblastic lymphoma, primary and secondary multiple myeloma, B-cell prolymphocytic leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, lymphomatoid granulomatosis, and acute lymphoblastic leukemia.

In some embodiments, the cancer is selected from chronic lymphocytic leukemia (CLL), diffuse large B-cell lymphoma (DLBCL), mantle cell lymphoma (MCL), small lymphocytic lymphoma (SLL), and Waldenstrom's macroglobulinemia. In one embodiment, the cancer is chronic lymphocytic leukemia (CLL). In another embodiment, the cancer is diffuse large B-cell lymphoma (DLBCL).

In certain embodiments, the present disclosure relates to the aforementioned methods, wherein said subject is a mammal. In certain embodiments, the present disclosure relates to the aforementioned methods, wherein said subject is a primate. In certain embodiments, the present disclosure relates to the aforementioned methods, wherein said subject is a human.

According to the disclosure an “effective dose” or an “effective amount” of the compound or pharmaceutical composition is that amount effective for treating or lessening the severity of one or more of the diseases, disorders or conditions as recited above. The effective dose of a compound provided herein, or a pharmaceutically acceptable salt thereof, administered to a subject can be 10 μg-500 mg.

The formulations may be prepared using conventional dissolution and mixing procedures. For example, the bulk drug substance (i.e., compound of the present disclosure or stabilized form of the compound (e.g., complex with a cyclodextrin derivative or other known complexation agent)) is dissolved in a suitable solvent in the presence of one or more of the excipients described above. The compound of the present disclosure is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethylene glycol; for tablets also c) binders, e.g., magnesium aluminum silicate, starch paste, gelatin, tragacanth, methylcellulose, sodium carboxymethylcellulose and/or polyvinylpyrrolidone; if desired d) disintegrants, e.g., starches, agar, alginic acid or its sodium salt, or effervescent mixtures; and/or e) absorbents, colorants, flavors and sweeteners. Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations.

Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compounds and compositions, according to the methods of the present disclosure, may be administered using any amount and any route of administration effective for treating or lessening the severity of one or more of the diseases, disorders or conditions recited above.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

The compound of the present disclosure or pharmaceutical composition thereof for use in a subject (e.g., human) is typically administered orally or parenterally at a therapeutic dose of less than or equal to about 100 mg/kg, 75 mg/kg, 50 mg/kg, 25 mg/kg, 10 mg/kg, 7.5 mg/kg, 5.0 mg/kg, 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

In certain embodiments, the present disclosure relates to the aforementioned methods, wherein said compound is administered intramuscularly, intravenously, subcutaneously, orally, pulmonary, rectally, intrathecally, topically or intranasally. The active compound may also be administered intravenously or intraperitoneally by infusion or injection. Solutions of the active compound or its salts can be prepared in water, optionally mixed with a nontoxic surfactant. In certain embodiments, the present disclosure relates to the aforementioned methods, wherein said compound is administered parenterally. In certain embodiments, the present disclosure relates to the aforementioned methods, wherein said compound is administered systemically.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The compound or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

### IV. Exemplifications

A. Abbreviations and acronyms used herein include the following:
ACN: means acetonitrile (CH₃CN)
AcOH: means Acetic acid;
t-Amyl-OH: means 2-methylbutan-2-ol
Aq.: means aqueous;
Ar: means argon;
br: means broad;
tBuXPhos Pd G3 means [(2-Di-tert-butylphosphino-2′,4′,6′-triisopropyl-1,1′-biphenyl)-2-(2′-amino-1,1′-biphenyl)] palladium(II) methanesulfonate
° C.: means degrees Celsius;
CAN means ceric ammonium nitrate [(NH₄)₂Ce(NO₃)₆]
CDCl₃: means deutero-chloroform;
CDI: means 1,1′-carbonyldiimidazole;
CH₂Cl₂: means methylene chloride
CaCl₂): means Calcium chloride;
Cs₂CO₃: means cesium carbonate;
d: means doublet;
dd: means double doublet;
δ: means chemical shift;
D₂O: means deuterated water;
DBU: means 1,8-Diazabicyclo[5.4.0]undec-7-ene;
DCM: dichloromethane;
DDQ means 2,3-dichloro-5,6-dicyano-1,4-benzoquinone
DEA: diethylamine
Dess-Martin Periodinane means 3-Oxo-1λ⁵,2-benziodoxole-1,1,1(3H)-triyl triacetate
DIPEA: diisopropyl ethylamine;
DMF: dimethylformamide
DMSO: means dimethylsulfoxide;
DMSO-d₆: means hexadeuterodimethyl sulfoxide;
ESI: electrospray ionization
Et: means ethyl;
Et₃N means triethylamine
EtOH: ethanol;
EtOAc: means ethyl acetate;
g: means gram;
h: means hour;
HATU: means 1-[bis(dimethylamino)methylene]-1H-1,2,3-triazolo[4,5-b]pyridinium 3-oxid hexafluorophosphate;
HBr: means hydrogen bromide;
HCO₂H: means formic acid;
HCl: means hydrochloric acid;
HPLC: means high pressure liquid chromatography;
¹H NMR: means proton nuclear magnetic resonance;
H₂O: means water;
IPA: means isopropyl alcohol;
K₂CO₃: means potassium carbonate;
KOH: means potassium hydroxide;
L: means litre;
LC: means liquid chromatography;
LC-MS: means liquid chromatography mass spectrometry;
LDA means lithium diisopropylamide
m: means multiplet;
M: means molar;
mins: means minutes;
mL: means millilitres;
μL: means micro litres;
mmol: means millimole;
m/z: mass to charge ratio
mg: means milligram;
Me: means methyl;
MeCN: means acetonitrile;
MeOH: means methanol;
MeOH-d₄: means deutero-methanol;
MHz: means mega Hertz;
Min(s): minute(s)
MS m/z: means mass spectrum peak;
MTBE: means tert-butyl methyl ether;
M/V: means Mass volume ratio;
N2 or N₂: means nitrogen;
NH₃: means ammonia;
NH₄Cl means ammonium chloride
Na: means sodium;
NaH: means sodium hydride;
NaHCO₃: means sodium bicarbonate;
NaOH: means sodium hydroxide;
NaOCN means sodium cyanate
Na₂SO₄: means sodium sulfate;
NH₄Cl: means ammonium chloride;
NH₄OAc means ammonium acetate
NH₄HCO₃: means ammonium bicarbonate;
NH₄OH: is ammonium hydroxide;
Pd₂(dba)₃: means Tris(dibenzylideneacetone)dipalladium(O);
Pd(dppf)Cl₂: means [1,1′-bis(diphenylphosphino)ferrocene]dichloropalladium(II);
Pd-PEPPSI-IHeptCl means Dichloro[1,3-bis(2,6-di-4-heptylphenyl)imidazol-2-ylidene](3-chloropyridyl)palladium(II)
Pd(t-Bu₃P)₂ means Bis(tri-ter-butylphosphine)palladium(0)
PE or Pet ether: means petroleum ether;
Psi: means pounds per square inch;
PTSA means p-Toluenesulfonic acid monohydrate
q: means quartet;
R_{f} means retention factor
RT: or means room temperature;
RuPhos means 2-Dicyclohexylphosphino-2′,6′-diisopropoxybiphenyl
s: means singlet;
sat.: means saturated;
soln.: means solution;
SFC: means supercritical fluid chromatography;
t: means triplet;
TEA: means triethylamine;
TFA: means trifluoroacetic acid;
THF: means tetrahydrofuran;
TLC: means thin layer chromatography;
μmol: means micromole;
UPLC means ultra performance liquid chromatography
V: volumes
XPhos: means 2-dicyclohexylphosphino-2′,4′,6′-triisopropylbiphenyl.

### B. General Methods

The compounds of the Examples were analyzed or purified according to one of the purification methods referred to below unless otherwise described.

Where preparative TLC or silica gel chromatography have been used, one skilled in the art may choose any combination of solvents to purify the desired compound. Silica gel column chromatography was performed using 20-40 μM (particle size), 100-200 mesh, 250-400 mesh, or 400-632 mesh silica gel using either a Teledyne ISCO Combiflash® RF, a Biotage® Isolera One 3.3.0, a Biotage® Flash Isolera Prime, a Grace Reveleris X2 with ELSD purification, a Gilson-281 with ELSD purification systems or using pressurized nitrogen (˜10-15 psi) to drive solvent through the column (“flash chromatography”).

Except where otherwise noted, reactions were run under an atmosphere of nitrogen. Where indicated, solutions and reaction mixtures were concentrated by rotary evaporation under vacuum.

### C. Analytical Methods

### NMR

### Instrument Specifications:

Bruker AVANCE III 400
Bruker AVANCE III HD 400
Bruker AVANCE NEO 400

### LC/MS

### Instrument Specifications:

Agilent 1200 Series LC/MSD system with DAD and Agilent LC\MS G6110A, mass-spectrometer.

Agilent(Degasser:1200;Pump:1260;Hip-ALS:1200;TCC:1200;DAD:1100)

Series LC/MS system with DAD\ELSD and Agilent LC\MS G6110A, mass-spectrometer.

Agilent(Degasser:1200;Pump:1260;Hip-ALS:1100;TCC:1260;DAD:1100)

Series LC/MS system with DAD and Agilent LC\MS G1956A, mass-spectrometer.

Agilent(Degasser:1200;Pump:1200;Hip-ALS:1100;TCC:1200;DAD:1200)

Series LC/MS system with DAD and Agilent LC\MS G1956A, mass-spectrometer.

Agilent 1290 Infinity II-6130 Quadrupole MS (single Quad)

SHIMADZU LC-20AD Series LC/MS system with SPD-M20A and SHIMADZU LC\MS LCMS-2020, mass-spectrometer.

SHIMADZU LC-20AD Series LC/MS system with SPD-M20A\ELSD and

SHIMADZU LC\MS LCMS-2020, mass-spectrometer

SHIMADZU LC-20AD Series LC/MS system with SPD-M40 and SHIMADZU LC\MS LCMS-2020, mass-spectrometer.

SHIMADZU LC-20AB Series LC/MS system with SPD-M20A and SHIMADZU LC\MS LCMS-2020, mass-spectrometer.

SHIMADZU LC-20AB Series LC/MS system with SPD-M20A\ELSD and SHIMADZU LC\MS LCMS-2020, mass-spectrometer.

Waters Acquity UPLC H-Class-SQ Detector 2

Ultima 3000 Dionex UHPLC-Thermo LCQ fleet ion trap

### HPLC

### Instrument Specifications:

SHIMADZU LC-20AD Series LC system with SPD-M20A

SHIMADZU LC-20AB Series LC system with SPD-M40

SHIMADZU LC-20AB Series LC system with SPD-M20A

Waters Acquity HPLC (binary/Quaternary Pump)

Agilent 1260 Infinity II LC system with PDA detector

### Prep-HPLC

### Instrument Specifications:

Shimadzu Nexera Prep-Pump-LC-20 AP with auto sampler and auto fraction collector

Gilson 331/332 HPLC pump system

Waters-MS prep-QDA

### SFC

### Instrument Specifications:

Waters 150/200 purification system

Waters investigator

Waters UPC2

### Sepiatec Screening System

Typically, the compounds of Formula (A) can be prepared according to the schemes provided below. The following examples serve to illustrate the disclosure without limiting the scope thereof. Methods for preparing such compounds are described hereinafter.

The disclosure further includes any variant of the present processes, in which the reaction components are used in the form of their salts or optically pure material. Compounds of the disclosure and intermediates can also be converted into each other according to methods generally known to those skilled in the art.

### D. LC-MS Methods

### Method 1

0.1% Formic acid in water (Aqueous phase)

100% Acetonitrile (Organic Phase)

Mode: gradient % B (5 to 95 in 3.7 minute)

Run Time: 4.8 minute

Column: Acquity UPLC BEH/X-Bridge BEH C18, 1.7 μm/2.5 μm, 2.1×50 mm

Flow rate: 0.5 mL/0.6 mL per minute

Temp: 40° C.

### Method 2

10 mM Ammonium Acetate in water (Aqueous phase)

100% Acetonitrile (Organic Phase)

Mode: gradient % B (5 to 95 in 3.7 minute)

Run Time: 4.8 minute

Column: Acquity UPLC BEH/X-Bridge BEH C18, 1.7 μm/2.5 μm, 2.1×50 mm

Flow rate: 0.5 mL/0.6 mL per minute

Temp: 40° C.

### Method 3

0.1% TFA in water (Aqueous phase)

100% Acetonitrile (Organic Phase)

Mode: gradient % B (5 to 95 in 3.7 minute)

Run Time: 4.8 minute

Column: Acquity UPLC BEH/X-Bridge BEH C18, 1.7 μm/2.5 μm, 2.1×50 mm

Flow rate: 0.5 mL/0.6 mL per minute

Temp: 40° C.

### Method 4

10 mM Ammonium Bicarbonate in water (Aqueous phase)

100% Acetonitrile (Organic Phase)

Mode: gradient % B (5 to 95 in 3.7 minute)

Run Time: 4.8 minute

Column: Acquity UPLC BEH/X-Bridge BEH C18, 1.7 μm/2.5 μm, 2.1×50 mm

Flow rate: 0.5 mL/0.6 mL per minute

Temp: 40° C.

### Method 5

Mobile phase: A: 0.0375% TFA in H₂O v/v

B: 0.01875% TFA in ACN, v/v

Column: Kinetex EVO C18 30*2.1 mm, 5 μm

Flow rate: 1.5 mL/min

Temp: 50° C.

Gradient: 5-95% B, 0-60% B, 30-90% B, or 50-100% B in 1.55 min

### Method 6

Mobile phase: A: 0.025% NH₃H₂O in H₂O, v/v
B: ACN

Column: Kinetex EVO C18 30*2.1 mm, 5 μm

Flow rate: 1.5 mL/min

Temp: 50° C.

Gradient: 5-95% B, 0-60% B, 30-90% B, or 50-100% B in 1.55 min

### E. Synthesis of Degradation Signaling Moieties

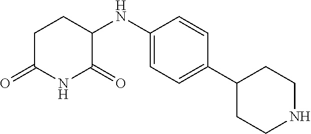 [SMILES: O=C1CCC(Nc2ccc(C3CCNCC3)cc2)C(=O)N1]

Intermediate 3-((4-(piperidin-4-yl)phenyl)amino)piperidine-2,6-dione was prepared according to the method described in page 267 of WO2018237026A1.

### Synthesis of 3-[3-fluoro-4-(4-piperidyl)anilino]piperidine-2,6-dione

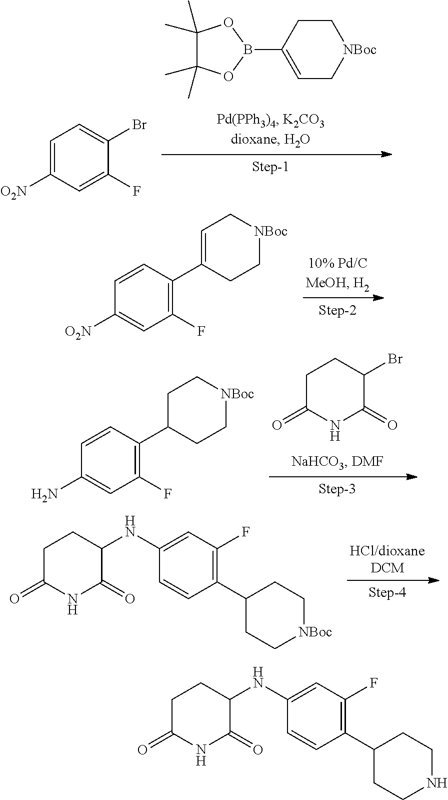

### Step-1:

A solution of 1-bromo-2-fluoro-4-nitro-benzene (6 g, 27.27 mmol) and tert-butyl 4-(4,4,5,5-tetramethyl-1,3,2-dioxaborolan-2-yl)-3,6-dihydro-2H-pyridine-1-carboxylate (8.43 g, 27.27 mmol) in dioxane (60 mL) and water (15 mL) in a round bottom flask was purged with argon gas for 10 minutes, followed by the addition of potassium carbonate, granular (11.31 g, 81.82 mmol). The solution was purged with argon gas for another 20 minutes before palladium;triphenylphosphane (1.58 g, 1.36 mmol) was added and the reaction was stirred at 90° C. for 16 hours. The progress of the reaction was monitored by TLC and LC-MS. After completion of the reaction, the reaction mixture was filtered through celite bed and washed with ethyl acetate. The filtrate was concentrated under reduced pressure and the crude product was diluted with water and extracted with ethyl acetate (2×150 ml). The combined organic layer was concentrated in vacuo and purified by normal phase column chromatography (Davisil silica, 5% ethyl acetate in pet ether) to obtain tert-butyl 4-(2-fluoro-4-nitro-phenyl)-3,6-dihydro-2H-pyridine-1-carboxylate (5.95 g, 18.27 mmol, 67.01% yield) as a light yellow solid. LC-MS (ES⁺): m/z 267.15 [M-tBu+H]⁺.

### Step-2:

To a stirred solution of tert-butyl 4-(2-fluoro-4-nitro-phenyl)-3,6-dihydro-2H-pyridine-1-carboxylate (3 g, 9.31 mmol) in methanol (70 mL) was added palladium, 10% on carbon, type 487, dry (3 g, 28.19 mmol) at room temperature. The reaction mixture was stirred for 6 hours at this temperature under hydrogen atmosphere, and the reaction progress was monitored by LC-MS. After completion of reaction, the reaction mixture was filtered through celite and the filtrate was concentrated under reduced pressure to afford compound tert-butyl 4-(4-amino-2-fluoro-phenyl)piperidine-1-carboxylate (2.5 g, 5.95 mmol, 63.88% yield) as purple solid, which was taken to the next step without purification. LC-MS (ES⁺): m/z 239.30 [M-tBu+H]⁺.

### Step-3:

In a sealed tube, a solution of tert-butyl 4-(4-amino-2-fluoro-phenyl)piperidine-1-carboxylate (2.5 g, 8.49 mmol) and 3-bromopiperidine-2,6-dione (4.08 g, 21.23 mmol) in DMF (40 mL) was stirred for 10 minutes before sodium bicarbonate (3.57 g, 42.46 mmol) was added and the reaction was heated at 60° C. for 16 hours. The progress of reaction was monitored by LC-MS and TLC. After completion of the reaction, the reaction mixture was filtered and concentrated in vacuo. The crude product was purified by column chromatography (Devisil silica, 0-30% ethyl acetate in pet ether) to furnish tert-butyl 4-[4-[(2,6-dioxo-3-piperidyl)amino]-2-fluoro-phenyl]piperidine-1-carboxylate (1.8 g, 3.64 mmol, 42.86% yield) as a brown solid. LC-MS (ES⁻): m/z 404.3 [M−H]⁻.

### Step-4:

To a solution of tert-butyl 4-(4-((2,6-dioxopiperidin-3-yl)amino)-2-fluorophenyl)piperidine-1-carboxylate (100 mg, 246.63 μmol) in DCM (1 mL) was added HCl/dioxane (2 mL). The mixture was stirred at 25° C. for 0.5 hour. After completion of the reaction as confirmed by LC-MS, the solvent was removed and the residue was dissolved in MeCN (30 mL), adjusted to pH=7 with NaHCO₃, and filtered. The filtrate was concentrated in vacuo and used in the next step directly. Compound 3-[3-fluoro-4-(4-piperidyl)anilino]piperidine-2,6-dione (75 mg, 233.34 μmol, 94.61% yield) was obtained as a white solid. LC-MS (ES⁺): m/z 306.2 [M+H]⁺.

### Synthesis of 3-[3-(4-piperidyl)anilino]piperidine-2,6-dione

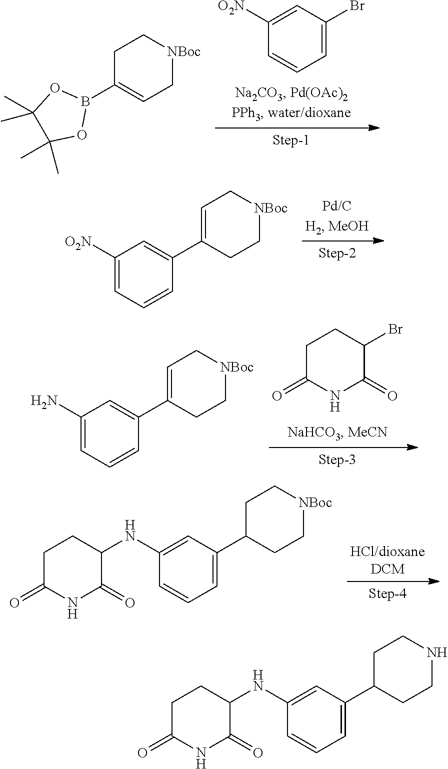

### Step-1:

To a solution of 1-bromo-3-nitrobenzene (5 g, 24.75 mmol) and tert-butyl 4-(4,4,5,5-tetramethyl-1,3,2-dioxaborolan-2-yl)-5,6-dihydropyridine-1(2H)-carboxylate (8.42 g, 27.23 mmol) in water (15 mL) and dioxane (50 mL) were added sodium carbonate (7.87 g, 74.26 mmol) and palladium acetate (555.70 mg, 2.48 mmol). The mixture was stirred at 90° C. for 12 hours. After LC-MS showed consumption of the reactant, the reaction mixture was diluted with water (80 mL) and extracted with ethyl acetate (50 mL×3). The combined organic layers were washed with brine (100 mL), dried over Na₂SO₄, filtered, and concentrated under reduced pressure. The residue was purified by column chromatography (silica gel, petroleum ether/ethyl acetate=1/0 to 1/1). Compound tert-butyl 4-(3-nitrophenyl)-5,6-dihydropyridine-1(2H)-carboxylate (6.5 g, 16.87 mmol, 68.17% yield) was obtained as a yellow solid. LC-MS (ES⁺): m/z 249.1 [M-tBu+H]⁺.

### Step-2:

To a solution of tert-butyl 4-(3-nitrophenyl)-3,6-dihydro-2H-pyridine-1-carboxylate (4 g, 13.14 mmol) in methanol (50 mL) was added 10 wt. % Pd/C (400 mg). The mixture was stirred at 25° C. for 5 hours under H₂ atmosphere (15 psi) and the reaction was monitored by TLC. Upon completion of the reaction, the reaction mixture was filtered, and the filtrate was concentrated in vacuo. Compound tert-butyl 4-(3-aminophenyl)piperidine-1-carboxylate (3.5 g, 12.66 mmol, 96.35% yield) was obtained as a white solid. ¹H NMR (400 MHz, DMSO-d₆) δ=6.91 (t, J=7.7 Hz, 1H), 6.51-6.22 (m, 3H), 4.92 (s, 2H), 4.03 (br d, J=12.1 Hz, 2H), 2.90-2.64 (m, 2H), 2.49-2.43 (m, 1H), 1.68 (br d, J=12.6 Hz, 2H), 1.40 (s, 10H).

### Step-3:

To a solution of tert-butyl 4-(3-aminophenyl)piperidine-1-carboxylate (2.5 g, 9.05 mmol) and 3-bromopiperidine-2,6-dione (1.74 g, 9.05 mmol) in MeCN (3 mL) was added NaHCO₃ (2.28 g, 27.14 mmol) and the mixture was stirred at 90° C. for 12 hours. After 73% of the desired product was detected by LC-MS, the reaction mixture was concentrated under reduced pressure to remove the solvent. The residue was diluted with water (20 mL) and extracted with ethyl acetate (20 mL×3). The combined organic layers were washed with brine (50 mL), dried over Na₂SO₄, filtered and concentrated under reduced pressure. The residue was purified by column chromatography (silica gel, petroleum ether/ethyl acetate=1/0 to 0/1). Compound tert-butyl 4-(3-((2,6-dioxopiperidin-3-yl)amino)phenyl)piperidine-1-carboxylate (2.5 g, 6.45 mmol, 71.33% yield) was obtained as a yellow solid. LC-MS (ES⁺): m/z 332.0 [M-tBu+H]⁺.

### Step-4:

To stirred solution of tert-butyl 4-[3-[(2,6-dioxo-3-piperidyl)amino]phenyl]piperidine-1-carboxylate (160 mg, 412.93 μmol) in DCM (2 mL) was added 4M HCl in dioxane (4 M, 1.03 mL) at 0° C. and the reaction was stirred at room temperature for 3 hours. After completion of the reaction, the solvent was removed under reduced pressure. The residue was washed with MTBE (10 mL×2) and then dried under reduced pressure to afford crude 3-[3-(4-piperidyl)anilino]piperidine-2,6-dione HCl salt (120 mg, 351.24 μmol, 85.06% yield) as a pale yellow solid. LC-MS (ES⁺): m/z 288.4 [M+H]⁺.

### Synthesis of 3-((6-(piperidin-4-yl)pyridin-3-yl)amino)piperidine-2,6-dione

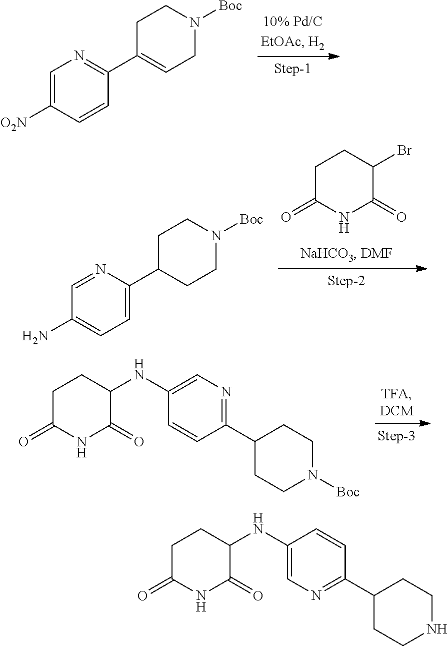

### Step-1:

To a stirred solution of tert-butyl 4-(5-nitro-2-pyridyl)-3,6-dihydro-2H-pyridine-1-carboxylate (10 g, 32.75 mmol) in ethyl acetate (100 mL) was added 10 wt. % palladium on carbon, type 487, dry (3.49 g, 32.75 mmol) and the reaction was stirred under hydrogen atmosphere for 16 hours. The reaction progress was monitored by TLC and LC-MS. After completion, the reaction mixture was filtered through a pad of celite and the filtrate was concentrate to dryness. The resulting crude product was purified by column chromatography(silica gel 60-120 mesh, 0-30% ethyl acetate in pet ether) to afford tert-butyl 4-(5-amino-2-pyridyl)piperidine-1-carboxylate (7 g, 23.47 mmol, 71.66% yield). LC-MS (ES⁻): m/z 276.24 [M−H]⁻.

### Step-2:

To a stirred solution of tert-butyl 4-(5-amino-2-pyridyl)piperidine-1-carboxylate (6.5 g, 23.44 mmol) and 3-bromopiperidine-2,6-dione (13.50 g, 70.31 mmol) in DMF (40 mL) was added sodium bicarbonate (19.69 g, 234.35 mmol) in a sealed tube. The reaction mixture was stirred at 85° C. for 16 hours. The progress of the reaction was monitored by TLC and LC-MS. Upon completion of the reaction, the reaction mixture was poured into ice water and the product was extracted with ethyl acetate. The organic layer was washed with cold brine solution, dried over anhydrous sodium sulfate and concentrated to give the crude product, which was purified by column chromatography (silica gel 230-400 mesh, 0-100% ethyl acetate in pet ether) to afford tert-butyl 4-[5-[(2,6-dioxo-3-piperidyl)amino]-2-pyridyl]piperidine-1-carboxylate (2.84 g, 6.40 mmol, 27.32% yield) as a light green solid. LC-MS (ES⁻): m/z 387.28 [M−H]⁻.

### Step-3:

To a stirred solution of tert-butyl 4-[5-[(2,6-dioxo-3-piperidyl)amino]-2-pyridyl]piperidine-1-carboxylate (1 g, 2.57 mmol) in DCM (10 mL) was added TFA (5.92 g, 51.92 mmol, 4 mL) at 0° C. The reaction mixture was stirred for 3 hours and the reaction progress was monitored by TLC and LC-MS. Upon completion of the reaction, the reaction mixture was evaporated to obtain the crude product, which was triturated with diethyl ether and concentrated in vacuo to afford 3-[[6-(4-piperidyl)-3-pyridyl]amino]piperidine-2,6-dione (700 mg, 2.03 mmol, 78.74% yield) as a green solid. LC-MS (ES⁺): m/z 289.46 [M+H]⁺.

### Synthesis of 3-[4-(3,3-difluoro-4-piperidyl)anilino]piperidine-2,6-dione

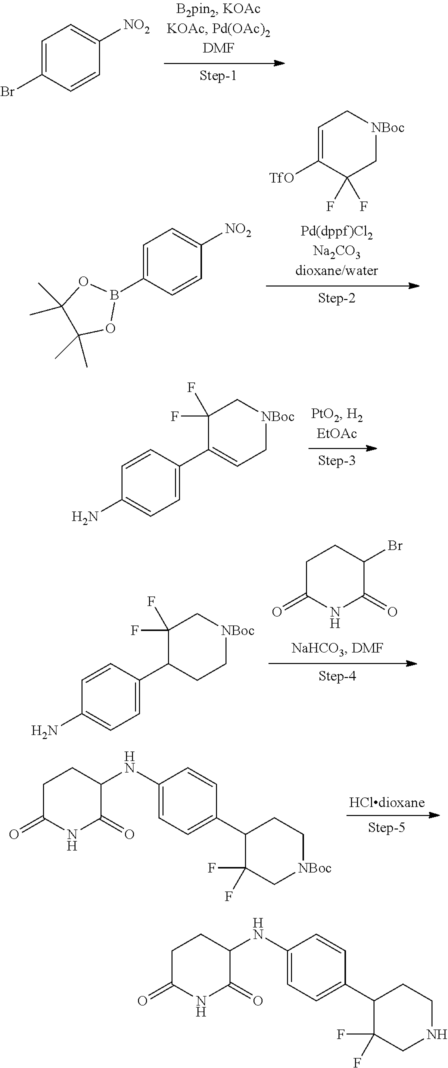

### Step-1:

To a stirred a solution of 1-bromo-4-nitro-benzene (5 g, 24.75 mmol, 2.56 mL) in DMF (40 mL) was added 4,4,5,5-tetramethyl-2-(4,4,5,5-tetramethyl-1,3,2-dioxaborolan-2-yl)-1,3,2-dioxaborolane (6.91 g, 27.23 mmol) and potassium acetate (6.07 g, 61.88 mmol). The resulting mixture was purged with argon gas for 30 minutes before palladium acetate (166.71 mg, 742.55 μmol) was added and reaction was refluxed at 60° C. for 6 hours. After completion of the reaction as indicated by TLC, the mixture was poured into cold water (100 mL) and the resulting solid was filtered and dried under high vacuum to afford 4,4,5,5-tetramethyl-2-(4-nitrophenyl)-1,3,2-dioxaborolane (3.5 g, 9.84 mmol, 39.74% yield) as a brown-black solid. 1H NMR (400 MHz, CDCl3) δ 8.19 (d, J=8.8 Hz, 2H), 7.96 (d, J=8.8 Hz, 2H), 1.37 (s, 12H).

### Step-2:

In a sealed tube, a solution of tert-butyl 3,3-difluoro-4-(trifluoromethylsulfonyloxy)-2,6-dihydropyridine-1-carboxylate (8.0 g, 21.78 mmol) and 4,4,5,5-tetramethyl-2-(4-nitrophenyl)-1,3,2-dioxaborolane (7.05 g, 28.32 mmol) in 1,4-dioxane (80 mL) were added sodium carbonate (4.62 g, 43.56 mmol) and cyclopentyl(diphenyl)phosphane; dichloropalladium; iron (1.59 g, 2.18 mmol) under argon atmosphere. The resulting mixture was stirred at 55 C for 3 hours, and progress of the reaction was monitored by TLC and LC-MS. After completion of the reaction, it was washed with water and extracted with ethyl acetate (3×250 mL). The combined organic layer was dried over anhydrous sodium sulfate and concentrated under reduced pressure. The crude product was purified by column chromatography (silica gel 230-400 mesh, EtOAc in pet ether) to afford tert-butyl 3,3-difluoro-4-(4-nitrophenyl)-2,6-dihydropyridine-1-carboxylate (4.4 g, 11.64 mmol, 53.42% yield) as a gummy solid. H NMR (400 MHz, CDCl3) δ 8.27 (d, J=8.8 Hz, 2H), 7.74 (d, J=8.8 Hz, 2H), 6.83 (bs, 1H), 4.22 (bs, 2H), 3.97 (t, J=6.8 Hz, 2H).

### Step-3:

To a stirred solution of tert-butyl 3,3-difluoro-4-(4-nitrophenyl)-2,6-dihydropyridine-1-carboxylate (9.0 g, 26.45 mmol) in ethyl acetate (100 mL) were added platinum (IV) oxide (6.01 g, 26.45 mmol). The reaction flask was evacuated and back filled with hydrogen gas using a hydrogen bladder and the reaction was stirred under hydrogen atmosphere at room temperature for 16 hours. After completion of the reaction as shown by TLC, the reaction mixture was filtered through celite bed and the filtrate was concentrated and purified by column chromatography (silica gel, ethyl acetate/pet ether) to afford tert-butyl 4-(4-aminophenyl)-3,3-difluoro-piperidine-1-carboxylate (5.4 g, 14.63 mmol, 55.31% yield) as a white solid. LC-MS (ES⁺): m/z 257.2 [M-tBu+H]⁺.

### Step-4:

To a stirred solution of tert-butyl 4-(4-aminophenyl)-3,3-difluoro-piperidine-1-carboxylate (5.0 g, 16.01 mmol) and 3-bromopiperidine-2,6-dione (9.22 g, 48.02 mmol) in DMF (50 mL) was added sodium bicarbonate (8.07 g, 96.04 mmol) at room temperature. The reaction mixture was stirred at 80° C. for 16 hours. Progress of the reaction was monitored by TLC and LC-MS. After completion, the reaction was quenched with water (100 mL) and extracted with EtOAc (3×100 mL). The combined organic layer was dried over anhydrous Na₂SO₄ and concentrated in vacuo. The crude compound was purified by column chromatography (silica gel 100-200 mesh, 15% EtOAc in pet ether) to afford tert-butyl 4-[4-[(2,6-dioxo-3-piperidyl)amino]phenyl]-3,3-difluoro-piperidine-1-carboxylate (5.17 g, 11.77 mmol, 73.54% yield). LC-MS (ES⁻): m/z 422.24 [M−H]⁻.

### Step-5:

To a stirred solution of tert-butyl 4-[4-[(2,6-dioxo-3-piperidyl)amino]phenyl]-3,3-difluoro-piperidine-1-carboxylate (0.5 g, 1.18 mmol) in dioxane (2 mL) was added HCl (4 M, 5 mL) under nitrogen atmosphere. The reaction was stirred at 0-28° C. for 2 hours and monitored by TLC and LC-MS. After completion of the reaction, the reaction mixture was concentrated to dryness and washed with diethyl ether(10 mL×2) to afford 3-[4-(3,3-difluoro-4-piperidyl)anilino]piperidine-2,6-dione HCl salt (0.4 g, 1.06 mmol, 89.45% yield) as a solid. LC-MS (ES⁺): m/z 324.09 [M+H]⁺.

### Synthesis of 1-(4-(piperidin-4-yl)benzyl)dihydropyrimidine-2,4(1H,3H)-dione

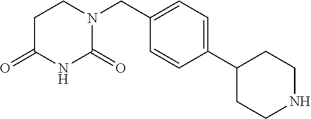 [SMILES: O=C1CCN(Cc2ccc(C3CCNCC3)cc2)C(=O)N1]

Intermediate 1-(4-(piperidin-4-yl)benzyl)dihydropyrimidine-2,4(1H,3H)-dione was prepared according to the method described on page 353 of WO2020132561A1.

### Synthesis of 3-[4-(4-piperidyl)phenoxy]piperidine-2,6-dione

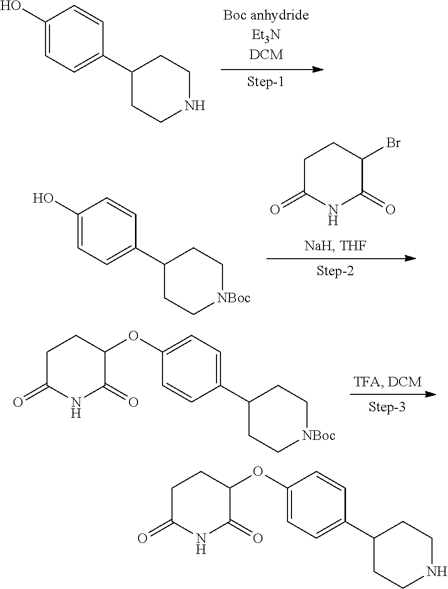

### Solution

A solution of 4-(4-piperidyl)phenol HBr salt (2.00 g, 7.75 mmol) in DCM (20 mL) was added into a 100 mL round bottom flask. Tert-butoxycarbonyl tert-butyl carbonate (2.03 g, 9.30 mmol, 2.13 mL) and triethylamine (3.92 g, 38.74 mmol, 5.40 mL) were added and the resulting mixture was stirred at room temperature for 2 hours. After completion of the reaction (confirmed by TLC), the reaction mixture was diluted with ethyl acetate (50 mL), and consecutively washed with water (20 mL) and brine (20 mL). The organic layer was dried over anhydrous sodium sulfate, filtered and concentrated to obtain the crude product, which was purified by flash column chromatography (silica gel 230-400 mesh, 0-80% ethyl acetate in pet ether) to afford tert-butyl 4-(4-hydroxyphenyl)piperidine-1-carboxylate (1.8 g, 6.45 mmol, 83.22% yield) as a white solid. LC-MS (ES⁺): m/z 178.2 [M-Boc+H]⁺.

### Step-2:

Sodium hydride (93.78 mg, 3.61 mmol) was added slowly to a stirred solution of tert-butyl 4-(4-hydroxyphenyl)piperidine-1-carboxylate (1.0 g, 3.61 mmol) in THE (10 mL) at 0° C. After addition, the reaction mixture was heated at 70° C. for 30 minutes. It was cooled 0° C. again before 3-bromopiperidine-2,6-dione (553.83 mg, 2.88 mmol) was added very slowly, after which the reaction mixture was heated at 70° C. for 2 hours. Progress of the reaction was monitored by TLC. Upon completion, the reaction was quenched by ammonium chloride and extracted with ethyl acetate, concentrated under reduce pressure to give the crude product, which was purified by column chromatography (silica gel 230-400 mesh, 0-50% ethyl acetate in pet-ether) to afford tert-butyl 4-[4-[(2,6-dioxo-3-piperidyl)oxy]phenyl]piperidine-1-carboxylate (0.5 g, 1.05 mmol, 29.17% yield). LC-MS (ES⁺): m/z 411.41 [M+Na]⁺.

### Step-3:

To a solution of tert-butyl 4-[4-[(2,6-dioxo-3-piperidyl)oxy]phenyl]piperidine-1-carboxylate (0.55 g, 1.42 mmol) in DCM (5 mL) was added. 2,2,2-trifluoroacetic acid (161.44 mg, 1.42 mmol, 109.08 μL) at 0° C. and the reaction mixture was stirred at room temperature for 1 hour. The reaction mixture was then concentrated in vacuo to give the crude product, which was triturated with diethyl ether (20 mL) to afford 3-[4-(4-piperidyl)phenoxy]piperidine-2,6-dione TFA salt (0.5 g, 1.13 mmol, 80.02% yield) as a white solid. LC-MS (ES⁺): m/z 289.28 [M+H]⁺.

### Synthesis of 3-[4-(2,5-diazaspiro[3.4]octan-5-ylmethyl)phenoxy]piperidine-2,6-dione

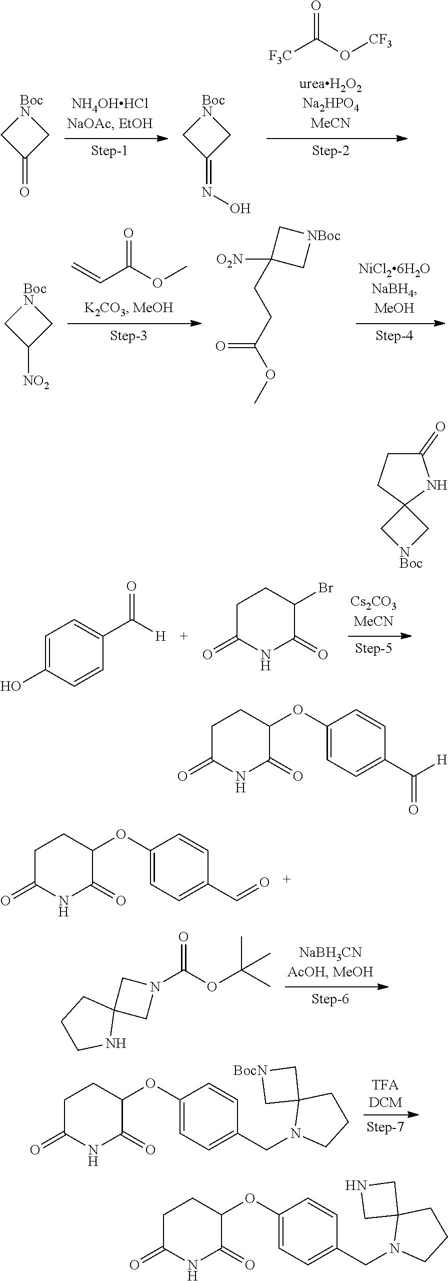

### Step-1:

To a stirred solution of tert-butyl 3-oxoazetidine-1-carboxylate (200 g, 1.17 mol) in ethanol (2000 mL), hydroxylamine hydrochloride (162.37 g, 2.34 mol, 97.23 mL) and sodium acetate, anhydrous (383.33 g, 4.67 mol) were added. The reaction mixture was stirred for 2 hours at 75-80° C. After completion of the reaction as indicated by TLC, the reaction was cooled to room temperature and filtered through celite. The filtrate was concentrated in vacuo and the crude product was extracted with ethyl acetate, washed with brine solution, dried over Na₂SO₄, and evaporated to afford tert-butyl 3-(hydroxyimino) azetidine-1-carboxylate (198 g, 1.02 mol, 87.38% yield) as a white crystalline solid. 1H NMR (400 MHz, DMSO-d6) δ 10.96 (s, 1H), 4.50 (d, J=10.8 Hz, 4H), 1.40 (s, 9H).

### Step-2:

To a stirred solution of tert-butyl 3-hydroxyiminoazetidine-1-carboxylate (135 g, 725.00 mmol) in acetonitrile (1800 mL), urea hydrogen peroxide (409.20 g, 4.35 mol) and disodium hydrogen phosphate (617.52 g, 4.35 mol) were added. Then (2,2,2-trifluoroacetyl) 2,2,2-trifluoroacetate (456.82 g, 2.17 mol, 306.59 mL) was added slowly (exotherm was observed) and the reaction mixture was heated to reflux for 3-4 hours at 60-70° C. After the reaction was complete as shown by TLC, the reaction was quenched with ice cold water and the mixture was extracted with ethyl acetate, washed with brine solution, dried over Na₂SO₄, and concentrated under reduced pressure. The residue was purified by column chromatography (15-20% ethyl acetate in pet ether) to afford tert-butyl 3-nitroazetidine-1-carboxylate (60 g, 280.89 mmol, 38.88% yield). 1H NMR (400 MHz, CDCl3) δ 5.20-5.10 (m, 1H), 4.43-4.30 (m, 4H), 1.45 (s, 9H).

### Step-3:

A stirred solution of tert-butyl 3-nitroazetidine-1-carboxylate (5 g, 24.73 mmol) in methanol (50 mL) was cooled to 0° C. Potassium carbonate (5.13 g, 37.09 mmol) followed by methyl acrylate (2.55 g, 29.67 mmol, 2.67 mL) were added to the reaction mixture at 0° C. and the reaction mixture was stirred for 3 hours at this temperature. After the completion of the reaction, the reaction mixture was concentrated under reduced pressure. The residue was diluted with aqueous saturated ammonium chloride solution and extracted with ethyl acetate. The organic layer was washed with brine, dried over Na₂SO₄, and concentrated under reduced pressure. The residue was purified by column chromatography (silica gel, 15-20% ethyl acetate in pet ether) to afford tert-butyl 3-(3-methoxy-3-oxo-propyl)-3-nitro-azetidine-1-carboxylate (4 g, 13.18 mmol, 53.31% yield) as a brown gummy solid. LC-MS (ES⁺): m/z [M+H]⁺. 1H NMR (400 MHz, CDCl3) δ 4.46 (d, J=10 Hz, 2H), 4.04 (d, J=10 Hz, 2H), 3.70 (s, 3H), 2.56-2.52 (m, 2H), 2.38-2.34 (m, 2H), 1.44 (s, 9H).

Step-4: A solution of tert-butyl 3-(3-methoxy-3-oxo-propyl)-3-nitro-azetidine-1-carboxylate (40 g, 138.75 mmol) in methanol (400 mL) was cooled to −10° C. and sodium borohydride (15.75 g, 416.24 mmol) was added. Then nickel(II) chloride hexahydrate, 98% (23.67 g, 83.25 mmol) was added portionwise over 1 hour (solution color changed from green to black). The reaction mixture was stirred for 1 hour at −10° C. After the reaction was complete as shown by TLC, the reaction was quenched with potassium carbonate solution (76.6 g in 80 mL water) at 0° C. The reaction mixture was stirred for 2 hours at room temperature, filtered through celite, and washed with ethyl acetate. The filtrate was extracted with ethyl acetate and washed with brine solution and dried over sodium sulfate. The organic layer was concentrated in vacuo to obtain a brown gummy liquid. The crude product was triturated with pentane and evaporated to give the product tert-butyl 2,5-diazaspiro[3.4]octane-2-carboxylate (25 g, 104.96 mmol, 75.65% yield). 1H NMR (400 MHz, DMSO-d6) δ 8.20 (s, 1H), 3.86 (s, 4H), 2.23-2.15 (m, 4H), 1.37 (s, 9H).

### Step-5:

To a stirred solution of 4-hydroxybenzaldehyde (20 g, 163.77 mmol, 17.70 mL) in ACN (300 mL) was added dicesium carbonate (160.08 g, 491.32 mmol) and the reaction was stirred for 30 minutes at 70° C. Then 3-bromopiperidine-2,6-dione (73.11 g, 380.77 mmol) was added to the reaction mixture and it was further stirred for 18 hours at 70° C. Progress of the reaction was monitored by TLC/LC-MS. After completion of the reaction, the solvent was removed under reduced pressure, and the crude product was diluted with water and extracted with ethyl acetate (3×200 mL). The combined organic layers were dried with anhydrous Na₂SO₄ and concentrated in vacuo. The crude compound was purified by column chromatography (Davisil silica, 40% ethyl acetate in pet ether) to afford 4-[(2,6-dioxo-3-piperidyl)oxy]benzaldehyde (10.26 g, 43.70 mmol, 26.68% yield) as an off-white solid. LC-MS (ES⁺): m/z 234.35 [M+H]⁺.

### Step-6:

In a sealed tube, a solution of 4-[(2,6-dioxo-3-piperidyl)oxy]benzaldehyde (0.250 g, 1.07 mmol), tert-butyl 2,5-diazaspiro[3.4]octane-2-carboxylate (227.56 mg, 1.07 mmol), acetic acid (0.250 g, 4.16 mmol, 238.10 μL) in methanol (3 mL) was stirred at 60° C. for 3 hours. Then the reaction was warmed up to room temperature and sodium cyanoborohydride (134.72 mg, 2.14 mmol) was added and stirred for 16 hours at this temperature. Progress of the reaction was monitored by LC-MS. After completion of the reaction, it was quenched with water. Subsequently, the reaction mixture was concentrated under reduced pressure to get the crude product, which was purified by reverse phase preparative HPLC to afford tert-butyl 5-[[4-[(2,6-dioxo-3-piperidyl)oxy]phenyl]methyl]-2,5-diazaspiro[3.4]octane-2-carboxylate (0.150 g, 345.74 μmol, 32.25% yield) as an off-white solid. LC-MS (ES⁺): m/z 430.42 [M+H]⁺.

### Step-7:

To a solution of tert-butyl 5-[[4-[(2,6-dioxo-3-piperidyl)oxy]phenyl]methyl]-2,5-diazaspiro[3.4]octane-2-carboxylate (0.150 g, 349.24 μmol) in DCM (2 mL) was added TFA (398.20 mg, 3.49 mmol, 269.05 μL) at 0° C. and the reaction mixture was stirred at room temperature for 4 hours. The reaction mixture was concentrated in vacuo to get the crude product, which was triturated with diethyl ether (5 mL) to afford 3-[4-(2,5-diazaspiro[3.4]octan-5-ylmethyl)phenoxy]piperidine-2,6-dione TFA salt (0.150 g, 312.30 μmol, 89.42% yield) as a brown semi solid. LC-MS (ES⁺): m/z 330.08 [M+H]⁺.

### Synthesis of 3-[4-(4-piperidyl)phenyl]piperidine-2,6-dione

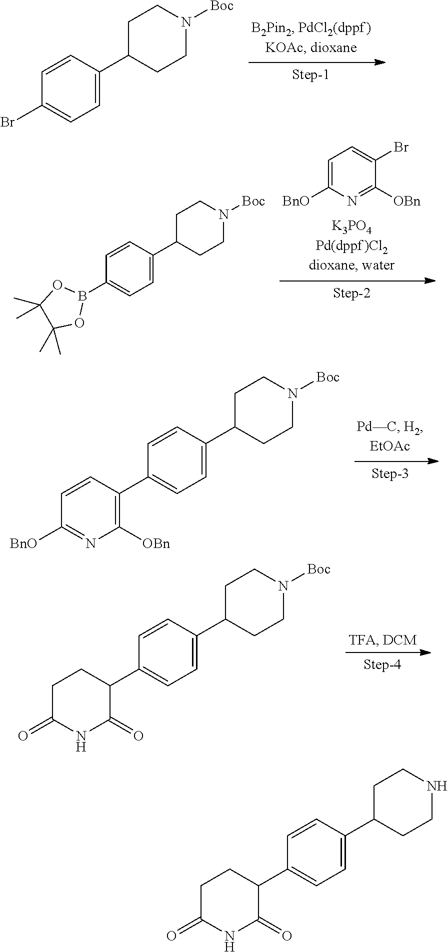 [SMILES: CC(C)(C)OC(=O)N1CCC(c2ccc(Br)cc2)CC1 ; Brc1ccc(OCc2ccccc2)nc1OCc1ccccc1 ; CC(C)(C)OC(=O)N1CCC(c2ccc(B3OC(C)(C)C(C)(C)O3)cc2)CC1 ; CC(C)(C)OC(=O)N1CCC(c2ccc(-c3ccc(OCc4ccccc4)nc3OCc3ccccc3)cc2)CC1 ; CC(C)(C)OC(=O)N1CCC(c2ccc(C3CCC(=O)NC3=O)cc2)CC1 ; O=C1CCC(c2ccc(C3CCNCC3)cc2)C(=O)N1]

### Step-1:

To a 500 mL round bottom flask was added a solution of tert-butyl 4-(4-bromophenyl)piperidine-1-carboxylate (10 g, 29.39 mmol) in 1,4 dioxane (100 mL) was added 4,4,5,5-tetramethyl-2-(4,4,5,5-tetramethyl-1,3,2-dioxaborolan-2-yl)-1,3,2-dioxaborolane (11.19 g, 44.08 mmol) followed by the addition of potassium acetate (8.65 g, 88.17 mmol) at room temperature under argon atmosphere. The reaction mixture was degassed with argon for 20 minutes, after which cyclopentyl(diphenyl)phosphane; dichloromethane; dichloropalladium; iron (2.40 g, 2.94 mmol) was added and the reaction was heated at 100° C. for 6 hours while monitoring with TLC and LC-MS. After completion of the reaction, the volatiles were removed under reduced pressure and the residue was extracted with ethyl acetate (200 mL×3) and water (200 mL). The combined organic layers were washed with brine solution (200 mL), dried over anhydrous sodium sulfate, filtered, and concentrated under reduced pressure. The crude product was purified by column chromatography (silica gel 100-200 mesh, 0-30% EtOAc in pet-ether) to afford tert-butyl 4-[4-(4,4,5,5-tetramethyl-1,3,2-dioxaborolan-2-yl)phenyl]piperidine-1-carboxylate (10 g, 24.27 mmol, 82.58% yield) as a pale yellow solid. LC-MS (ES⁺): m/z 332.41 [M−56+H]⁺.

### Step-2:

To a 500 mL round bottom flask was added a solution of tert-butyl 4-[4-(4,4,5,5-tetramethyl-1,3,2-dioxaborolan-2-yl)phenyl]piperidine-1-carboxylate (10 g, 25.82 mmol) in 1,4 dioxane (120 mL) and water (30 mL), followed by the addition of 2,6-dibenzyloxy-3-bromo-pyridine (10.04 g, 27.11 mmol) and potassium phosphate tribasic anhydrous (16.44 g, 77.46 mmol) at room temperature under argon atmosphere. The reaction mixture was degassed with argon for 20 minutes, after which cyclopentyl(diphenyl) phosphane; dichloropalladium; iron (1.89 g, 2.58 mmol) was added and the reaction was heated at 110° C. for 16 hours while monitoring with TLC and LC-MS. Upon completion of the reaction, the catalyst was filtered off through celite bed and washed with ethyl acetate (100 mL×3). The filtrate was washed with water (100 mL) and brine solution (100 mL). The combined organic layers were dried over anhydrous sodium sulfate, filtered, and concentrated under reduced pressure. The crude product was purified by column chromatography (silica gel 230-400 mesh, 0-40% ethyl acetate in pet-ether) to afford the desired product as a yellow thick liquid, which was triturated with pet ether to furnish pure tert-butyl 4-[4-(2,6-dibenzyloxy-3-pyridyl)phenyl]piperidine-1-carboxylate (7 g, 11.57 mmol, 44.80% yield) as a white color solid. LC-MS (ES⁺): m/z 551.43 [M+H]⁺.

Step-3: A solution of tert-butyl 4-[4-(2,6-dibenzyloxy-3-pyridyl)phenyl]piperidine-1-carboxylate (14 g, 25.42 mmol) in ethyl acetate (420 mL) was added 10% wt. palladium on charcoal (14 g, 25.42 mmol), and the reaction was stirred under hydrogen pressure (70 psi) at room temperature for 16 hours. The reaction progress was monitored by TLC and LC-MS. After the reaction was complete, the catalyst was filtered off through celite and washed with ethyl acetate (200 mL). The filtrate was concentrated under reduced pressure and the residue was triturated in pentane (100 mL) and diethyl ether (100 mL), dried, and concentrated under reduced pressure to afford tert-butyl 4-[4-(2,6-dioxo-3-piperidyl)phenyl]piperidine-1-carboxylate (8.6 g, 23.05 mmol, 90.65% yield) as a white solid. LC-MS (ES⁻): m/z 371.23 [M−H]⁻.

### Step-4:

To a stirred solution of tert-butyl 4-[4-(2,6-dioxo-3-piperidyl)phenyl]piperidine-1-carboxylate (250 mg, 671.22 μmol) in DCM (5 mL) was added TFA (5.92 g, 51.92 mmol, 4 mL) at 0° C. The reaction was stirred for 2 hours, and the reaction progress was monitored by LC-MS and TLC. Upon completion, the reaction mixture was concentrated in vacuo to yield the crude product, which was triturated with diethyl ether to obtained the desired product 3-[4-(4-piperidyl)phenyl]piperidine-2,6-dione TFA salt (250 mg, 404.22 μmol, 60.22% yield) as a brown liquid. LC-MS (ES⁻): m/z 371.23 [M−H]⁻.

### Synthesis of 3-(3-fluoro-4-(piperidin-4-yl)phenyl)piperidine-2,6-dione

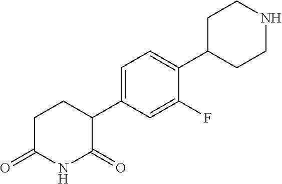 [SMILES: O=C1CCC(c2ccc(C3CCNCC3)c(F)c2)C(=O)N1]

The procedures were substantially similar to those of 3-[4-(4-piperidyl) phenyl]piperidine-2,6-dione, except the synthesis started with tert-butyl 4-(4-bromo-2-fluoro-phenyl)piperidine-1-carboxylate instead of tert-butyl 4-(4-bromophenyl)piperidine-1-carboxylate and palladium hydroxide was used instead of palladium for step-3. LC-MS (ES⁺): m/z 291.37 [M+H]⁺.

### Synthesis of 3-(2,5-difluoro-4-(piperidin-4-yl)phenyl)piperidine-2,6-dione

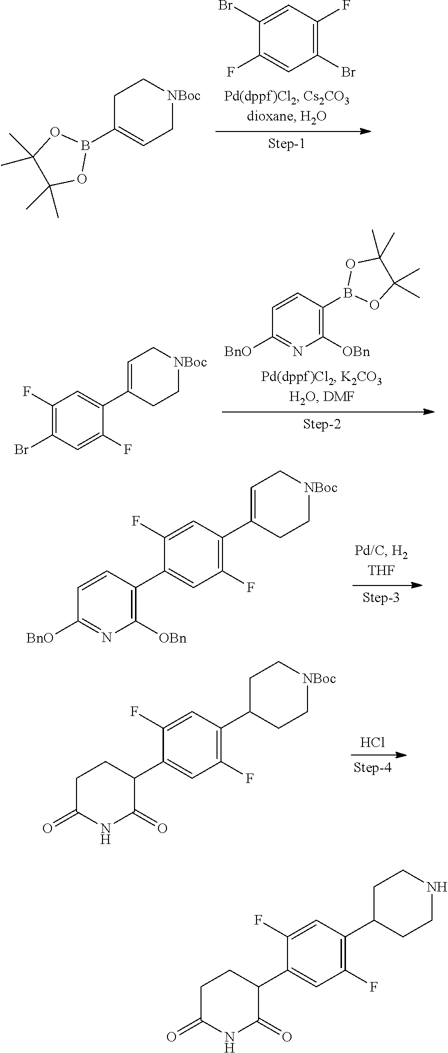

### Step-1:

A mixture of tert-butyl 4-(4,4,5,5-tetramethyl-1,3,2-dioxaborolan-2-yl)-5,6-dihydropyridine-1(2H)-carboxylate (10 g, 32.34 mmol),1,4-dibromo-2,5-difluoro-benzene (9.67 g, 35.57 mmol), cyclopentyl(diphenyl)phosphane;dichloropalladium;iron (2.37 g, 3.23 mmol), cesium carbonate (42.15 g, 129.36 mmol) in dioxane (100 mL) and water (20 mL) was degassed and purged with N₂ three times. The mixture was stirred at 80° C. for 16 hours under N₂ atmosphere.

After completion of the reaction as confirmed by LC-MS, the suspension was filter through a pad of celite. The filtrate was diluted with water (200 mL×2), and extracted with ethyl acetate (200 mL×3). The combined organic layers were washed with brine (100 mL×2), dried over anhydrous sodium sulfate, filtered, and concentrated in vacuo. The residue was purified by column chromatography (silica gel, pet ether/ethyl acetate=10/1 to 5/1). Compound tert-butyl 4-(4-bromo-2,5-difluorophenyl)-5,6-dihydropyridine-1(2H)-carboxylate (5.16 g, 11.31 mmol, 34.96% yield) was obtained as a white solid. LC-MS (ES⁺): m/z 317.9 [M-tBu+H]⁺.

### Step-2:

To a solution of tert-butyl 4-(4-bromo-2,5-difluorophenyl)-5,6-dihydropyridine-1(2H)-carboxylate (4.7 g, 12.56 mmol) in water (10 mL) and 2,6-bis(benzyloxy)-3-(4,4,5,5-tetramethyl-1,3,2-dioxaborolan-2-yl)pyridine (5.24 g, 12.56 mmol) was added cyclopentyl (diphenyl)phosphane; dichloropalladium; iron (918.98 mg, 1.26 mmol) and potassium carbonate (5.21 g, 37.68 mmol). The mixture was stirred at 80° C. for 16 hours under nitrogen atmosphere. After complete consumption of the reactant as confirmed by LC-MS, the reaction mixture was diluted with water (150 mL) and extracted with ethyl acetate (100 mL×3). The combined organic layers were washed with brine (100 mL), dried over anhydrous sodium sulfate, filtered, and concentrated under reduced pressure. The residue was purified by column chromatography (silica gel, pet ether/ethyl acetate=100/1 to 10/1). Compound tert-butyl 4-(4-(2,6-bis(benzyloxy)pyridin-3-yl)-2,5-difluorophenyl)-5,6-dihydropyridine-1(2H)-carboxylate (6.4 g, 10.95 mmol, 87.16% yield) was obtained as a light-yellow solid. LC-MS (ES⁺): m/z 585.3 [M+H]⁺.

### Step-3:

To a solution of tert-butyl 4-(4-(2,6-bis(benzyloxy)pyridin-3-yl)-2,5-difluorophenyl)-5,6-dihydropyridine-1(2H)-carboxylate (6.4 g, 10.95 mmol) in THE (60 mL) was added Pd/C (1.75 g, 1.64 mmol, 0.1 purity) under N₂ atmosphere. The suspension was degassed and purged with H₂ three times. The mixture was stirred under H₂ (15 Psi) at 25° C. for 5 hours. After complete consumption of the reactant as indicated by LC-MS, the reaction mixture was filtered and the filtrate was concentrated to give a solid. The crude product was used in the next step without further purification. Compound tert-butyl 4-(4-(2,6-dioxopiperidin-3-yl)-2,5-difluorophenyl)piperidine-1-carboxylate (4 g, 6.29 mmol, 57.49% yield) was obtained as an off-white solid. LC-MS (ES⁺): m/z 353.1 [M-tBu+H]⁺.

### Step-4:

A solution of tert-butyl 4-[4-(2,6-dioxo-3-piperidyl)-2,5-difluoro-phenyl]piperidine-1-carboxylate (4 g, 9.79 mmol) and HCl (16.00 g, 438.83 mmol, 20 mL) were stirred at 25° C. for 2 hours. After completion of the reaction as shown by TLC, the reaction mixture was concentrated under reduced pressure to remove solvent. The residue was used in the next step without further purification. Compound 3-(2,5-difluoro-4-(piperidin-4-yl)phenyl)piperidine-2,6-dione HCl salt (3.4 g, 9.76 mmol, 99.69% yield) was obtained as an off-white solid. LC-MS (ES⁺): m/z 309.2 [M+H]⁺.

### Synthesis of 3-[4-(3,3-difluoro-4-piperidyl)phenyl]piperidine-2,6-dione

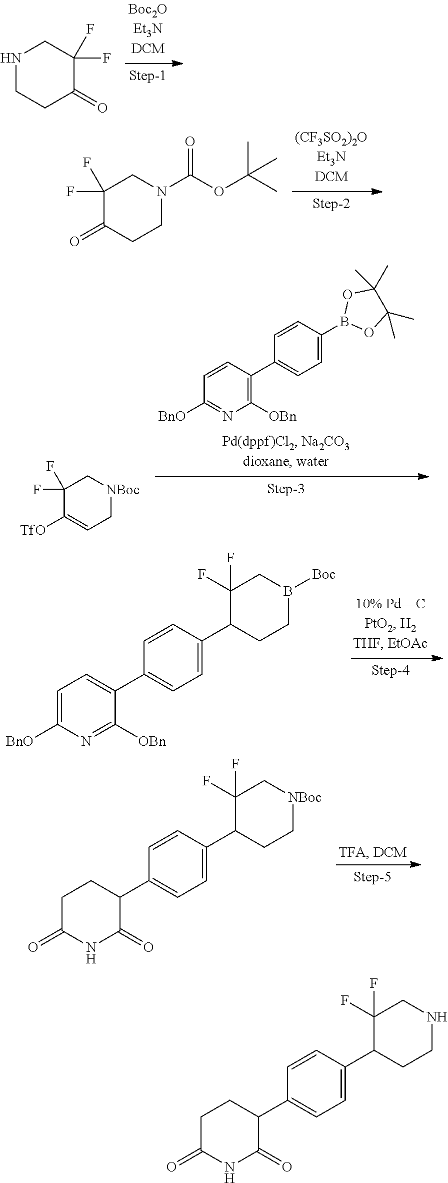

### Step-1:

To a stirred solution of 3,3-difluoropiperidin-4-one (0.5 g, 3.70 mmol) in DCM (10 mL) was added triethylamine (561.70 mg, 5.55 mmol, 773.69 μL) and reaction mixture was stirred for 10 minutes. Tert-butoxycarbonyl tert-butyl carbonate (969.18 mg, 4.44 mmol, 1.02 mL) was then added and stirred at room temperature for 16 hours. Progress of the reaction was monitored by TLC and LC-MS. Upon completion, the reaction was quenched by addition of water (10 mL) and stirred for 5 min. The mixture was then extracted with DCM (2×10 mL), and the organic layer was washed with 10 mL of brine, dried over anhydrous sodium sulfate, and concentrated under reduced pressure to obtain the crude product as a brown gummy material. (700 mg, 48.25% yield). ¹H NMR (400 MHz, DMSO-d₆) δ 6.38 (s, 2H), 3.60 (t, J=11.6 Hz, 2H), 3.37 (bs, 2H), 1.68 (bs, 2H), 1.39 (s, 9H). The compound is in hydrate form.

### Step-2:

To a stirred solution of tert-butyl 3,3-difluoro-4-oxo-piperidine-1-carboxylate (5 g, 21.26 mmol) in DCM (50 mL) was added triethylamine (6.45 g, 63.77 mmol, 8.89 mL) and the reaction was stirred for 1 hour at −30° C. This is followed by the addition of trifluoromethylsulfonyl trifluoromethanesulfonate (9.00 g, 31.88 mmol, 5.36 mL) and the reaction was stirred −30° C. for 16 hours and monitored by LC-MS and TLC. Upon completion, the reaction was quenched with water (3×50 ml) and extracted with DCM (3×50 ml). The organic layer was dried over sodium sulfate and concentrated under reduced pressure to yield a crude product, which was purified by column chromatography (Devisil-silica, 7% ethyl acetate/petroleum ether) to afford compound tert-butyl 3,3-difluoro-4-(trifluoromethylsulfonyloxy)-2,6-dihydropyridine-1-carboxylate (1.8 g, 4.42 mmol, 20.80% yield) as a yellow gummy liquid. LC-MS (ES⁺): m/z 268.16 [M−100+H]⁺.

### Step-3:

To the stirred solution of tert-butyl 3,3-difluoro-4-(trifluoromethylsulfonyloxy)-2,6-dihydropyridine-1-carboxylate (3.5 g, 9.53 mmol) and 2,6-dibenzyloxy-3-[4-(4,4,5,5-tetramethyl-1,3,2-dioxaborolan-2-yl)phenyl]pyridine (5.64 g, 11.44 mmol) in dioxane (40 mL) water (10 mL) was added sodium carbonate (2.52 g, 23.82 mmol). The mixture was degassed with N₂ and cyclopentyl(diphenyl)phosphane;dichloropalladium;iron (697.26 mg, 952.93 μmol) was added at room temperature. The reaction was stirred for 12 hours at 60° C., and the progress was monitored by TLC and LC-MS. After the reaction was complete, it was diluted with water (50 mL) and extracted with ethyl acetate (150 mL×3). The combined organic layers were dried over sodium sulfate and concentrated under reduced pressure to yield the crude product, which was purified by column chromatography (20-30% ethyl acetate in pet ether) to afford tert-butyl 4-[4-(2,6-dibenzyloxy-3-pyridyl)phenyl]-3,3-difluoro-2,6-dihydropyridine-1-carboxylate (2.0 g, 2.84 mmol, 29.80% yield) as a brown solid. LC-MS (ES⁺): m/z 585.44 [M+H]⁺.

### Step-4:

To the stirred solution of tert-butyl 4-[4-(2,6-dibenzyloxy-3-pyridyl)phenyl]-3,3-difluoro-2,6-dihydropyridine-1-carboxylate (2 g, 3.42 mmol) in THF (40 mL), ethyl acetate (10 mL) was added 10 wt. % palladium on carbon wet (1.82 g, 17.10 mmol) and dioxoplatinum (932.15 mg, 4.11 mmol). The reaction was stirred for 12 hours at room temperature under hydrogen atmosphere, and the reaction progress was monitored by the TLC and LC-MS. After completion, the reaction mixture was filtered through celite using ethyl acetate and filtrate was concentrated under reduced pressure to yield the crude product, which was triturated with diethyl ether. The diethyl ether layer was decanted and desired product was dried under reduced pressure to afford tert-butyl 4-[4-(2,6-dioxo-3-piperidyl)phenyl]-3,3-difluoro-piperidine-1-carboxylate (995 mg, 2.22 mmol, 64.92% yield). LC-MS (ES⁻): m/z 407.12 [M−H]⁻.

### Step-5:

To a stirred solution of tert-butyl 4-[4-(2,6-dioxo-3-piperidyl)phenyl]-3,3-difluoro-piperidine-1-carboxylate (0.1 g, 244.84 μmol) in DCM (2 mL) was added TFA (4.44 g, 38.94 mmol, 3 mL) under nitrogen and the reaction was stirred at 0-28° C. for 2 hours. The reaction progress was monitored by TLC and LC-MS. Upon completion, the reaction was evaporated to dryness and washed with diethyl ether(10 mL×2) to afford 3-[4-(3,3-difluoro-4-piperidyl)phenyl]piperidine-2,6-dione TFA salt (85 mg, 100.63 μmol, 41.10% yield) as a solid. LC-MS (ES⁺): m/z 309.00 [M+H]⁺.

### Synthesis of 3-[4-(3,3-difluoro-4-piperidyl)-2,5-difluoro-phenyl]piperidine-2,6-dione

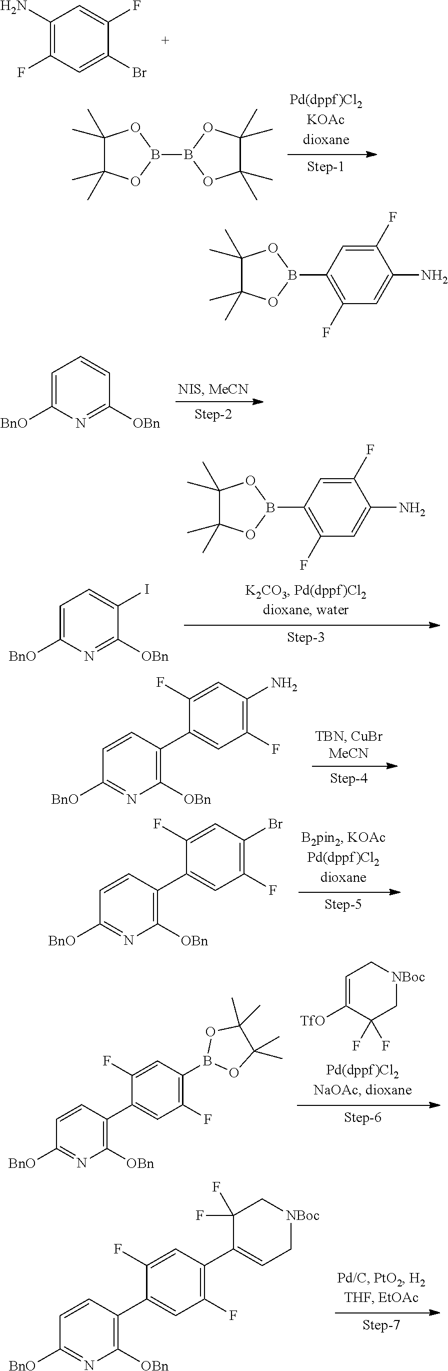

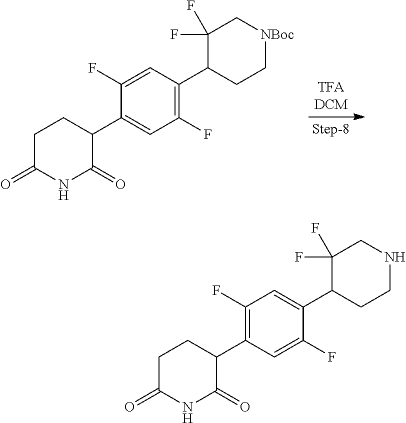 [SMILES: CC(C)(C)OC(=O)N1CCC(c2cc(F)c(C3CCC(=O)NC3=O)cc2F)C(F)(F)C1 ; O=C1CCC(c2cc(F)c(C3CCNCC3(F)F)cc2F)C(=O)N1]

### Step-1:

To a solution of 4-bromo-2,5-difluoro-aniline (5.2 g, 25.00 mmol) and 4,4,5,5-tetramethyl-2-(4,4,5,5-tetramethyl-1,3,2-dioxaborolan-2-yl)-1,3,2-dioxaborolane (12.70 g, 50.00 mmol) in dioxane (3 mL) was added potassium acetate (7.36 g, 75.00 mmol) at room temperature. The reaction mixture was degassed with argon for 10 minutes and cyclopentyl(diphenyl)phosphane; dichloromethane; dichloropalladium; iron (1.02 g, 1.25 mmol) was added. The reaction mixture was degassed with argon for an additional 5 minutes and it was stirred at 100° C. for 12 hours. Subsequently, the reaction mixture was concentrated in vacuo to get the crude product, which was purified by column chromatography (davisil silica, 12% ethyl acetate in pet ether) to afford 2,5-difluoro-4-(4,4,5,5-tetramethyl-1,3,2-dioxaborolan-2-yl)aniline (7 g, 11.36 mmol, 45.46% yield) as a pale yellow solid. LC-MS (ES⁺): m/z 255.46 [M+H]⁺.

Step-2: To a stirred solution of 2,6-dibenzyloxypyridine (6 g, 20.59 mmol) in acetonitrile (200 mL) was added 1-iodopyrrolidine-2,5-dione (4.63 g, 20.59 mmol) slowly at 0° C. The reaction was then warmed up and stirred at 80° C. for 2 hours, while monitoring by LCMS and TLC. After completion of the reaction, the reaction mixture was concentrated in vacuo and extracted with cold water (100 ml) and ethyl acetate (200 ml). The combined organic layers were washed with water, brine, dried over anhydrous Na₂SO₄, filtered, and concentrated under reduced pressure. The crude compound was washed with pentane to afford the product 2,6-dibenzyloxy-3-iodo-pyridine (6 g, 9.06 mmol, 43.99% yield) as a pale-yellow solid. LC-MS (ES⁺): m/z 418.28 [M+H]⁺.

Step-3: In a sealed tube, a solution of 2,6-dibenzyloxy-3-iodo-pyridine (10 g, 23.97 mmol) and 2,5-difluoro-4-(4,4,5,5-tetramethyl-1,3,2-dioxaborolan-2-yl)aniline (7.34 g, 28.76 mmol) in dioxane (30 mL) and water (0.3 mL) was added potassium carbonate, anhydrous, 99% (9.94 g, 71.90 mmol) at room temperature. The reaction mixture was degassed with argon for 10 minutes before cyclopentyl(diphenyl)phosphane;dichloropalladium;iron (1.75 g, 2.40 mmol) was added. The reaction mixture was degassed with argon for an additional 5 minutes and it was stirred at 110° C. for 16 hours. Subsequently, the reaction mixture was concentrated in vacuo to get the crude product, which was purified by column chromatography (silica gel 200-400 mesh, 10% ethyl acetate in pet ether) to afford 4-(2,6-dibenzyloxy-3-pyridyl)-2,5-difluoro-aniline (6 g, 12.90 mmol, 53.83% yield) as a pale-yellow solid. LC-MS (ES⁺): m/z 419.22 [M+H]⁺.

### Step-4:

A solution of copper(I) bromide (2.06 g, 14.34 mmol, 436.72 μL), tert-butyl nitrite (2.96 g, 28.68 mmol, 3.41 mL) in acetonitrile (50 mL) was cooled to 0° C. Then, 4-(2,6-dibenzyloxy-3-pyridyl)-2,5-difluoro-aniline (6 g, 14.34 mmol) in acetonitrile (20 mL) and added to the reaction mixture at the same temperature. The reaction was warmed up to 25° C. slowly and stirred for 16 hours and monitored by TLC. After completion of the reaction, water (100 mL) was added to the reaction mixture and extracted with ethyl acetate (100 mL×2) and the organic layer was concentrated in vacuo to afford 2,6-dibenzyloxy-3-(4-bromo-2,5-difluoro-phenyl)pyridine (4.4 g, 7.27 mmol, 50.70% yield) as a pale yellow oil. LC-MS (ES⁺): m/z 482.28 [M+H]⁺.

### Step-5:

In a sealed tube, a solution of 2,6-dibenzyloxy-3-(4-bromo-2,5-difluoro-phenyl)pyridine (4.4 g, 9.12 mmol) and (4,4,5,5-tetramethyl-2-(4,4,5,5-tetramethyl-1,3,2-dioxaborolan-2-yl)-1,3,2-dioxaborolane (4.63 g, 18.25 mmol) in dioxane (50 mL) was added potassium acetate (2.69 g, 27.37 mmol) at room temperature. The reaction mixture was degassed with argon for 10 minutes and cyclopentyl(diphenyl)phosphane; dichloropalladium; iron (333.75 mg, 456.13 μmol) was added. The reaction mixture was degassed with argon for an additional 5 minutes and then stirred at 110° C. for 16 hours. Subsequently, the reaction mixture was concentrated in vacuo to get the crude product, which was purified by column chromatography (Davisil silica, 10% ethyl acetate in pet ether) to afford 2,6-dibenzyloxy-3-[2,5-difluoro-4-(4,4,5,5-tetramethyl-1,3,2-dioxaborolan-2-yl)phenyl]pyridine (5.6 g, 4.97 mmol, 54.52% yield) as a pale brown oil. LC-MS (ES⁺): m/z 530.46 [M+H]⁺.

### Step-6:

In a sealed tube, a solution of tert-butyl 3,3-difluoro-4-(trifluoromethylsulfonyloxy)-2,6-dihydropyridine-1-carboxylate (3.0 g, 8.17 mmol) and 2,6-dibenzyloxy-3-[2,5-difluoro-4-(4,4,5,5-tetramethyl-1,3,2-dioxaborolan-2-yl)phenyl]pyridine (5.19 g, 9.80 mmol) in dioxane (120 mL) was added sodium acetate, anhydrous (2.01 g, 24.50 mmol) at room temperature. The reaction mixture was degassed with argon for 10 minutes before cyclopentyl(diphenyl) phosphane; dichloropalladium; iron (298.83 mg, 408.40 μmol) was added. The reaction mixture was degassed with argon for an additional 5 minutes and it was stirred at 110° C. for 16 hours. Subsequently, the reaction mixture was concentrated in vacuo to get the crude product, which was purified by column chromatography (silica gel 200-400 mesh, 12% ethyl acetate in pet ether) to afford tert-butyl 4-[4-(2,6-dibenzyloxy-3-pyridyl)-2,5-difluoro-phenyl]-3,3-difluoro-2,6-dihydropyridine-1-carboxylate (2.6 g, 3.25 mmol, 39.79% yield) as a pale brown solid. LC-MS (ES⁺): m/z 621.43 [M+H]⁺.

### Step-7:

To a stirred solution of tert-butyl 4-[4-(2,6-dibenzyloxy-3-pyridyl)-2,5-difluoro-phenyl]-3,3-difluoro-2,6-dihydropyridine-1-carboxylate (2.6 g, 4.19 mmol) in THE (20 mL) and ethyl acetate (80 mL), palladium, 10% on carbon, type 487, dry (445.82 mg, 4.19 mmol), platinum (IV) oxide hydrate (1.03 g, 4.19 mmol) were added to the reaction and the mixture was stirred under H₂ balloon for 16 hours, The reaction was monitored by LC-MS. After completion of the reaction, the reaction mixture was filtered through celite bed and washed with ethyl acetate (20 mL). The filtrate was concentrated under reduced pressure to give tert-butyl 4-[4-(2,6-dioxo-3-piperidyl)-2,5-difluoro-phenyl]-3,3-difluoro-piperidine-1-carboxylate (1.46 g, 1.95 mmol, 46.58% yield) as a pale brown sticky mass. LC-MS (ES⁻): m/z 443.41 [M−H]⁻.

### Step-8:

To a stirred solution of tert-butyl 4-[4-(2,6-dioxo-3-piperidyl)-2,5-difluoro-phenyl]-3,3-difluoro-piperidine-1-carboxylate (1.46 g, 3.29 mmol) in DCM (50 mL), trifluoroacetic acid (1.87 g, 16.43 mmol, 1.27 mL) was added to the reaction mixture and stirred at 25° C. for 16 hours. The reaction was monitored by LCMS. After completion of the reaction, the reaction mixture was concentrated in vacuo and the crude product was washed with 50% ethyl acetate in pet ether (70 mL) to afford 3-[4-(3,3-difluoro-4-piperidyl)-2,5-difluoro-phenyl]piperidine-2,6-dione TFA salt (0.6475 g, 1.26 mmol, 38.34% yield). LC-MS (ES⁺): m/z 345.15 [M+H]⁺.

### Synthesis of 3-methyl-3-[4-(4-piperidyl)phenyl]piperidine-2,6-dione

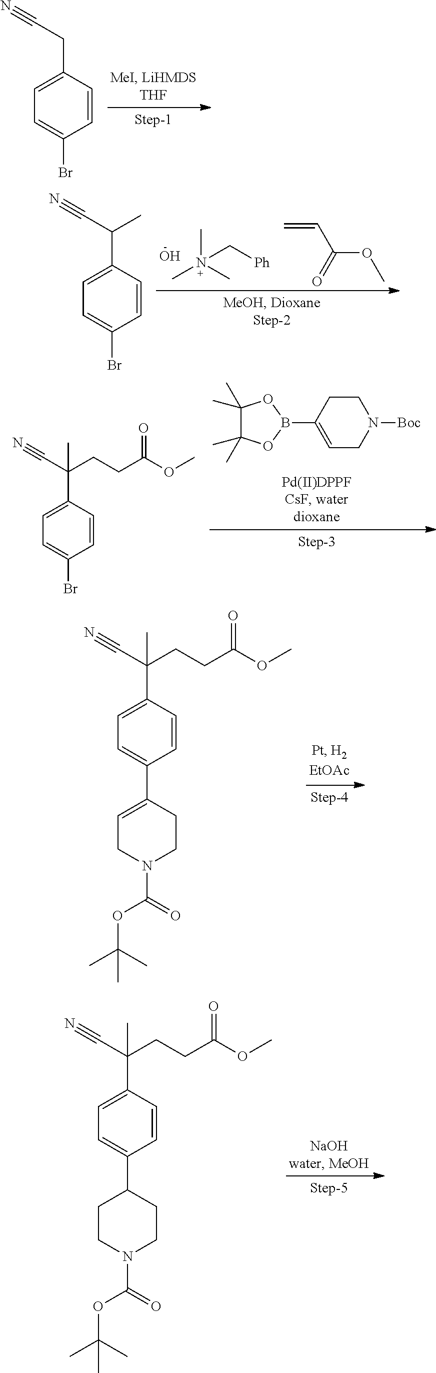

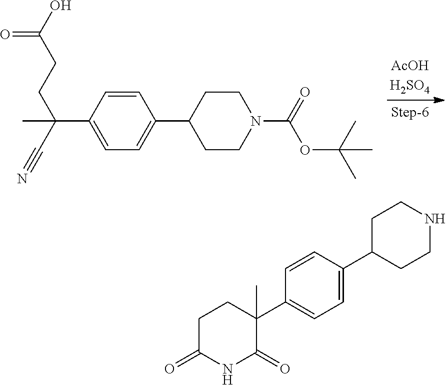

### Step-1:

To a solution of 2-(4-bromophenyl)acetonitrile (2 g, 10.20 mmol, 1.34 mL) in THE (20 mL) was added lithium bis(trimethylsilyl)amide (1 M, 12.24 mL) at −78° C. under an atmosphere of argon. The mixture was stirred at −78° C. for 0.5 hour then iodomethane (1.59 g, 11.22 mmol, 698.61 μL) was added, and the mixture was stirred at −78° C. for 2 hours. The reaction mixture was quenched by addition of ammonium chloride (50 mL) and extracted with ethyl acetate (50 mL*2). The combined organic layers were washed with brine (50 mL), dried over Na₂SO₄, filtered, and concentrated under reduced pressure. The residue was purified by flash silica gel chromatography (10 g silica, 0-10% ethyl acetate/petroleum ether gradient at 70 mL/min) to give 2-(4-bromophenyl)propanenitrile (1.41 g, 6.64 mmol, 65.13% yield) as a yellow oil. ¹H NMR (400 MHz, CDCl₃) δ 7.54-7.52 (m, 2H), 7.27-7.24 (m, 2H), 3.88 (q, J=7.2 Hz, 1H), 1.65-1.63 (d, J=7.2 Hz, 3H).

### Step-2:

To a solution of 2-(4-bromophenyl)propanenitrile (1 g, 4.76 mmol) in dioxane (10 mL) was added benzyltrimethylammonium hydroxide, 40% w/w in methanol (796.15 mg, 1.90 mmol) and 2-(4-bromophenyl)propanenitrile (1 g, 4.76 mmol) at 0° C. and the mixture was stirred at 25° C. for 4 hours. The reaction mixture was quenched by addition of ammonium chloride (20 mL) at 0° C. and extracted with ethyl acetate (50 mL×2). The combined organic layers were washed with brine (50 mL), dried over Na₂SO₄, filtered, and concentrated under reduced pressure to give methyl 4-(4-bromophenyl)-4-cyano-pentanoate (1.05 g, 3.51 mmol, 73.73% yield) as a yellow oil. ¹H NMR (400 MHz, CDCl₃) δ 7.47-7.45 (m, 2H), 7.26-7.24 (m, 2H), 3.56 (s, 3H), 2.42-2.14 (m, 4H), 1.66 (s, 3H).

### Step-3:

A mixture of methyl 4-(4-bromophenyl)-4-cyano-pentanoate (1.05 g, 3.55 mmol), tert-butyl 4-(4,4,5,5-tetramethyl-1,3,2-dioxaborolan-2-yl)-3,6-dihydro-2H-pyridine-1-carboxylate (1.32 g, 4.25 mmol), cyclopentyl(diphenyl)phosphane;dichloropalladium;iron (129.71 mg, 177.27 μmol) and fluorocesium (1.62 g, 10.64 mmol, 392.15 μL) in water (2 mL) and dioxane (10 mL) was degassed and purged with nitrogen three times, and then the mixture was stirred at 90° C. for 12 hours under a nitrogen atmosphere. The reaction mixture was quenched by addition of water (50 mL) and extracted with ethyl acetate (50 mL×2). The combined organic layers were washed with NaCl (50 mL), dried over Na₂SO₄, filtered, and concentrated under reduced pressure. The residue was purified by flash silica gel chromatography (10 g silica, 0-20% ethyl acetate in petroleum ether gradient at 60 mL/min) to give tert-butyl 4-[4-(1-cyano-4-methoxy-1-methyl-4-oxo-butyl)phenyl]-3,6-dihydro-2H-pyridine-1-carboxylate (1.3 g, 3.23 mmol, 91.09% yield) as a yellow oil. LC-MS (ES⁺): m/z 299.1 [M+H-Boc]⁺.

### Step-4:

To a solution of tert-butyl 4-[4-(1-cyano-4-methoxy-1-methyl-4-oxo-butyl)phenyl]-3,6-dihydro-2H-pyridine-1-carboxylate (1.3 g, 3.26 mmol) in ethyl acetate (20 mL) was added palladium, 5% on activated carbon paste (347.17 mg, 3.26 mmol) under a nitrogen atmosphere. The suspension was degassed and purged with hydrogen three times. The mixture was stirred under hydrogen at 25° C. for 4 hours. The reaction mixture was filtered and concentrated under reduced pressure. The product, tert-butyl 4-[4-(1-cyano-4-methoxy-1-methyl-4-oxo-butyl)phenyl]piperidine-1-carboxylate (1.3 g, 3.25 mmol, 99.50% yield), was used in the next step without further purification. LC-MS (ES⁺): m/z 423.3 [M+Na]⁺.

### Step-5:

To a solution of tert-butyl 4-[4-(1-cyano-4-methoxy-1-methyl-4-oxo-butyl)phenyl]piperidine-1-carboxylate (11.7 g, 29.21 mmol) in water (10 mL) and methanol (100 mL) was added sodium hydroxide, pearl (2.34 g, 58.43 mmol, 1.10 mL) and the mixture was stirred at 25° C. for 12 hours. The reaction mixture was concentrated under reduced pressure to remove MeOH, was diluted with H2O (50 mL), and extracted with ethyl acetate (100 mL×2). The water layer was adjusted pH with 1M HCl to 5, and extracted with DCM (100 mL×3), dried over Na₂SO₄, filtered and concentrated under reduced pressure to give 4-[4-(1-tert-butoxycarbonyl-4-piperidyl)phenyl]-4-cyano-pentanoic acid (9.5 g, 23.35 mmol, 79.94% yield) was as a white solid and was used in the next step without further purification. ¹H NMR (400 MHz, DMSO-d₆) δ=12.57-12.04 (m, 1H), 7.46-7.38 (m, 2H), 7.32 (d, J=8.4 Hz, 2H), 4.15-4.00 (m, 2H), 2.94-2.65 (m, 3H), 2.33-2.13 (m, 3H), 2.11-1.97 (m, 1H), 1.75 (br d, J=12.5 Hz, 2H), 1.67 (s, 3H), 1.55-1.44 (m, 2H), 1.42 (s, 9H).

### Step-6:

A mixture of 4-[4-(1-tert-butoxycarbonyl-4-piperidyl)phenyl]-4-cyano-pentanoic acid (6.5 g, 16.82 mmol), acetic acid (52.50 g, 874.27 mmol, 50 mL) and sulfuric acid (1.65 g, 16.82 mmol, 10 mL) was stirred at 100° C. for 6 hours. The reaction mixture was concentrated under reduced pressure and the residue was purified by reversed phase flash chromatography (flow: 100 mL/min; gradient: from 100-50% water in acetonitrile (with HCl modifier) over 15 min; column: 330 g Flash Column Welch Ultimate XB_C18 20-40 μm; 120 A) to give 3-methyl-3-[4-(4-piperidyl)phenyl]piperidine-2,6-dione hydrochloride (4.40 g, 13.07 mmol, 77.73% yield) as a yellow solid. ¹H NMR (400 MHz, DMSO-d₆) δ=10.94 (s, 1H), 9.10-8.74 (m, 2H), 7.28-7.21 (m, 4H), 3.36 (br s, 2H), 2.98 (br t, J=10.3 Hz, 2H), 2.88-2.78 (m, 1H), 2.49-2.41 (m, 1H), 2.40-2.32 (m, 1H), 2.14-2.02 (m, 2H), 1.93-1.82 (m, 4H), 1.42 (s, 3H).

### Synthesis of 3-fluoro-3-[4-(4-piperidyl)phenyl]piperidine-2,6-dione

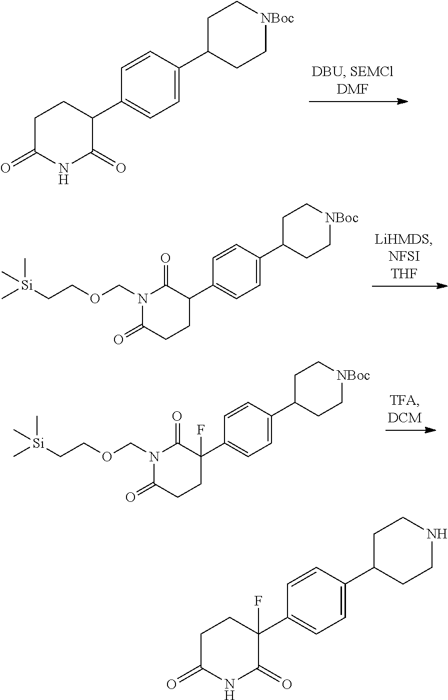 [SMILES: CC(C)(C)OC(=O)N1CCC(c2ccc(C3CCC(=O)NC3=O)cc2)CC1 ; CC(C)(C)OC(=O)N1CCC(c2ccc(C3CCC(=O)N(COCC[Si](C)(C)C)C3=O)cc2)CC1 ; CC(C)(C)OC(=O)N1CCC(c2ccc(C3(F)CCC(=O)N(COCC[Si](C)(C)C)C3=O)cc2)CC1 ; O=C1CCC(F)(c2ccc(C3CCNCC3)cc2)C(=O)N1]

### Step-1:

To a stirred solution of tert-butyl 4-[4-(2,6-dioxo-3-piperidyl)phenyl]piperidine-1-carboxylate (8.4 g, 22.55 mmol) in DMF (10 mL) were added 1,8-diazabicyclo[5.4.0]undec-7-ene (6.87 g, 45.11 mmol, 6.73 mL) and 2-(trimethylsilyl)ethoxymethyl chloride (5.64 g, 33.83 mmol, 5.99 mL) at 0° C. The reaction mixture was stirred at 25° C. for 16 hours. The progress of the reaction was monitored by TLC and LC-MS. After completion of the reaction, it was cooled to room temperature, diluted with water, and extracted with ethyl acetate. The combined organic phase was dried over anhydrous sodium sulfate, filtered and concentrated under reduced pressure. The crude product was purified by column chromatography (silica gel 100-200 mesh, 0-50% EtOAc in pet ether) to afford tert-butyl 4-(4-(2,6-dioxo-1-((2-(trimethylsilyl)ethoxy)methyl)piperidin-3-yl)phenyl)piperidine-1-carboxylate (6.2 g, 11.59 mmol, 51.40% yield) as yellow color gummy liquid. LC-MS (ES⁻): m/z 501.36 [M−H]⁻.

### Step-2:

To a stirred solution of tert-butyl 4-(4-(2,6-dioxo-1-((2-(trimethylsilyl)ethoxy) methyl)piperidin-3-yl)phenyl)piperidine-1-carboxylate (6.0 g, 11.94 mmol) in THE (120 mL) was added lithium bis(trimethylsilyl)amide (3.99 g, 23.87 mmol) and N-fluorobenzene sulfonimide (3.76 g, 11.94 mmol) at 0° C. The reaction mixture was stirred at −78° C. for 20 minutes. The progress of the reaction was monitored by TLC and LCMS. After completion of the reaction, the mixture was cooled to room temperature, quenched with NH₄Cl solution (200 mL), and extracted with ethyl acetate (500 mL). The combined organic phase was dried over anhydrous sodium sulfate, filtered, and concentrated under reduced pressure. The crude product was purified by prep-HPLC to afford tert-butyl 4-(4-(3-fluoro-2,6-dioxo-1-((2-(trimethylsilyl)ethoxy)methyl)piperidin-3-yl)phenyl)piperidine-1-carboxylate (1.34 g, 1.78 mmol, 14.88% yield) as yellow color gummy liquid. LC-MS (ES⁻): m/z 519.29 [M−H]⁻.

### Step-3:

To a solution of tert-butyl 4-[4-[3-fluoro-2,6-dioxo-1-(2-trimethylsilylethoxymethyl)-3-piperidyl]phenyl]piperidine-1-carboxylate (0.580 g, 1.11 mmol) in DCM (6 mL) was added TFA (1.27 g, 11.14 mmol, 858.13 μL) at 0° C. and the reaction mixture was stirred at room temperature for 16 hours. The reaction mixture was concentrated in vacuo to give the crude product, which was triturated with diethyl ether (50 mL) to afford 3-fluoro-3-[4-(4-piperidyl)phenyl]piperidine-2,6-dione TFA salt (0.580 g, 1.00 mmol, 90.14% yield) as an off-white semi solid. LC-MS (ES⁺): m/z 291.22 [M+H]⁺.

### Synthesis of 1-[4-(4-piperidyl)phenyl]hexahydropyrimidine-2,4-dione

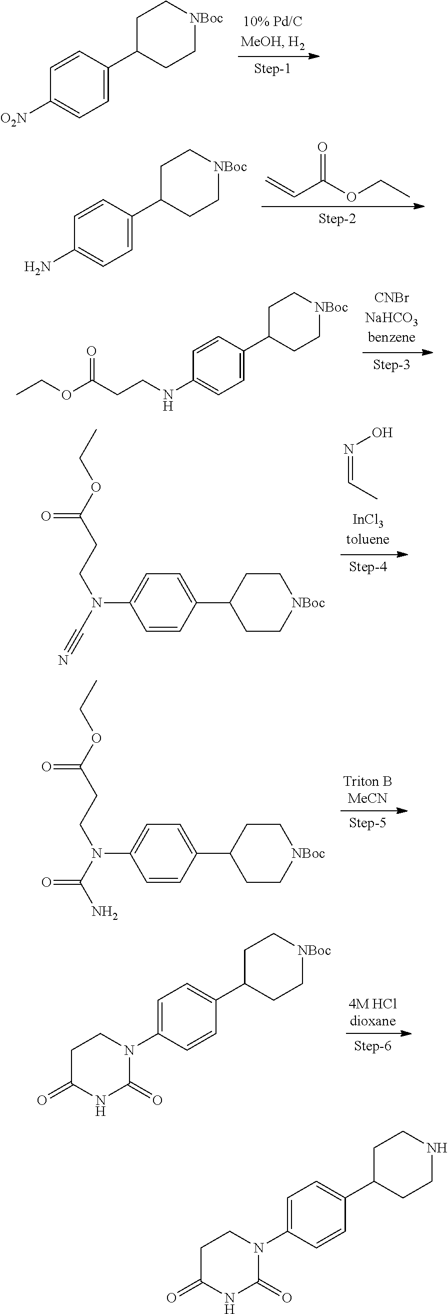

### Step-1:

A solution of tert-butyl 4-(4-nitrophenyl)-3,6-dihydro-2H-pyridine-1-carboxylate (15.0 g, 49.29 mmol) in methanol (300 mL) was degassed argon gas for 10 minutes. To the reaction mixture was added 10 wt. % palladium on carbon (10.49 g, 98.57 mmol) at room temperature and the hydrogenation was carried out at 70 psi using parr apparatus for 16 hours. The progress of the reaction was monitored by LC-MS. Upon completion, the reaction was filtered through celite bed and washed with methanol (4×20 mL). The organic layer was concentrated under reduced pressure at 45° C. to afford the desired product tert-butyl 4-(4-aminophenyl)piperidine-1-carboxylate (11.8 g, 34.14 mmol, 69.26% yield) as an off-white solid. which was taken to the next step without any further purification. LC-MS (ES⁺): m/z 177.17 [M−100+H]⁺.

### Step-2:

A mixture of tert-butyl 4-(4-aminophenyl)piperidine-1-carboxylate (16 g, 57.89 mmol), DBU lactic acid (ionic liquid) (10.28 g, 34.74 mmol) and ethyl acrylate (7.53 g, 75.26 mmol, 8.02 mL) was stirred at 90° C. for 3 hours. The progress of the reaction was monitored by TLC and LC-MS. Upon completion, the reaction was allowed cool to room temperature, and diluted with ethyl acetate. The aqueous layer was separated, and the organic layer was dried over anhydrous sodium sulfate and concentrated to yield the crude product, which was purified by CombiFlash® using 5-10% ethyl acetate in hexane as eluent to afford tert-butyl 4-[4-[(3-ethoxy-3-oxo-propyl)amino]phenyl]piperidine-1-carboxylate (12.5 g, 31.54 mmol, 54.48% yield) as a gummy yellow liquid. LC-MS (ES⁺): m/z 321.2 [M-tBu+H]⁺.

### Step-3:

To the stirred solution of tert-butyl 4-[4-[(3-ethoxy-3-oxo-propyl)amino]phenyl]piperidine-1-carboxylate (15 g, 39.84 mmol) in benzene (100 mL), carbononitridic bromide (6.75 g, 63.75 mmol, 3.34 mL) and sodium bicarbonate (5.36 g, 63.75 mmol) were added simultaneously and the reaction was stirred for 24 hours at room temperature. After complete consumption of the starting material as monitored by TLC, the reaction mixture was diluted with ethyl acetate (20 ml). The organic phase was washed with water, separated, dried over sodium sulfate and concentrated under vacuum to give a crude residue, which was purified by column chromatography to afford tert-butyl 4-[4-[cyano-(3-ethoxy-3-oxo-propyl)amino]phenyl]piperidine-1-carboxylate (12.5 g, 29.58 mmol, 74.24% yield).as a semi solid. LC-MS (ES⁺): m/z 402.2 [M+H]⁺.

### Step-4:

A stirred solution of tert-butyl 4-[4-[cyano-(3-ethoxy-3-oxo-propyl)amino]phenyl]piperidine-1-carboxylate (12.5 g, 31.13 mmol), trichloroindigane (2.07 g, 9.34 mmol) and (1Z)-acetaldehyde oxime (5.52 g, 93.40 mmol) in toluene (100 mL) was refluxed for 1 hour. After complete consumption of the starting material as monitored by TLC, the reaction mixture was concentrated in vacuo and washed with pentane to obtain tert-butyl 4-[4-[carbamoyl-(3-ethoxy-3-oxo-propyl)amino]phenyl]piperidine-1-carboxylate (12 g, 26.03 mmol, 83.61% yield) as a gummy liquid, which was used in the next step without further purification. LC-MS (ES⁺): m/z 364.4 [M-tBu+H]⁺.

### Step-5:

A solution of tert-butyl 4-[4-[carbamoyl-(3-ethoxy-3-oxo-propyl)amino]phenyl]piperidine-1-carboxylate (12 g, 28.60 mmol) in acetonitrile (120 mL) was heated at 60° C. with stirring. Triton B (40% in methanol) (17.94 g, 42.91 mmol, 19.50 mL) was added to the mixture and the reaction was stirred at the same temperature for 10 minutes. After complete consumption of the starting material (confirmed by TLC and LC-MS), the reaction mixture was concentrated in vacuo and the crude residue was purified by column chromatography to afford tert-butyl 4-[4-(2,4-dioxohexahydropyrimidin-1-yl)phenyl]piperidine-1-carboxylate (8 g, 21.21 mmol, 74.14% yield) as a white solid. LC-MS (ES⁺): m/z 318.1 [M-tBu+H]⁺.

### Step-6:

To a stirred suspension of tert-butyl 4-[4-(2,4-dioxohexahydropyrimidin-1-yl)phenyl]piperidine-1-carboxylate (13.50 g, 36.15 mmol) in dioxane (40 mL) was added 4 M HCl in dioxane (50 mL) at 0° C. and reaction mixture was stirred for 3 hours at room temperature. After completion of the reaction as evidenced from LC-MS, the volatiles are removed under vacuum to afford 1-[4-(4-piperidyl)phenyl]hexahydropyrimidine-2,4-dione HCl salt (11.1 g, 34.77 mmol, 96.18% yield) as a white solid. LC-MS (ES⁺): m/z 274.4 [M+H]⁺.

### Synthesis of 3-[4-(2,6-diazaspiro[3.3]heptan-2-yl)phenyl]piperidine-2,6-dione

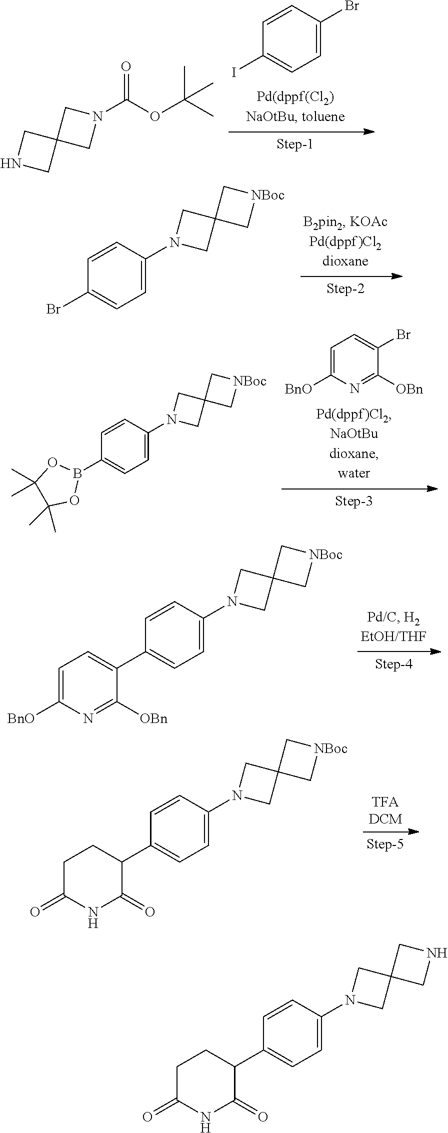

### Step-1:

In a 50 mL Schlenk tube, tert-butyl 2,6-diazaspiro[3.3]heptane-2-carboxylate (8.0 g, 40.35 mmol) and 1-bromo-4-iodo-benzene (11.42 g, 40.35 mmol) in toluene (80 mL) was degassed with nitrogen for 15 minutes. Then [1,1′-bis(diphenylphosphino) ferrocene]dichloropalladium(II), complex with dichloromethane (3.30 g, 4.04 mmol) and sodium tert-butoxide (19.39 g, 201.75 mmol) were added and the mixture was further degassed for 5 minutes. The reaction mixture was sealed and heated at 65° C. for 60 hours and monitored by TLC and UPLC. After complete consumption of the starting material, the solvent was removed under reduced pressure and the residue was purified by column chromatography (silica gel, 0-20% ethyl acetate in pet ether) to afford tert-butyl 6-(4-bromophenyl)-2,6-diazaspiro[3.3]heptane-2-carboxylate (8.5 g, 22.85 mmol, 56.64% yield) as off white solid. LC-MS (ES⁺): m/z 354.9 [M+H]⁺.

### Step-2:

In a sealed tube, a stirred solution of tert-butyl 6-(4-bromophenyl)-2,6-diazaspiro[3.3]heptane-2-carboxylate (5 g, 14.15 mmol) in dioxane (60 mL) was added bis(pinacolato)diboron (5.03 g, 19.82 mmol) followed by potassium acetate (4.17 g, 42.46 mmol). The reaction mixture was degassed with nitrogen for 10 minutes, followed by addition of Pd(dppf)Cl₂ (1.16 g, 1.42 mmol), and the mixture was degassed with nitrogen for 10 minutes, before being heated at 90° C. for 16 hours. The reaction progress was monitored by TLC and UPLC. After completion of the reaction, the reaction mixture was cooled to room temperature and the mixture was filtered through a pad of celite and washed with ethyl acetate. The filtrate was concentrated in vacuo to give the crude compound which was purified by column chromatography (silica gel 100-200 mesh) to afford tert-butyl 6-[4-(4,4,5,5-tetramethyl-1,3,2-dioxaborolan-2-yl)phenyl]-2,6-diazaspiro[3.3]heptane-2-carboxylate (5.1 g, 12.35 mmol, 87.24% yield) as a white solid. LC-MS (ES⁺): m/z 401.2 [M+H]⁺.

### Step-3:

To a stirred solution of 2,6-dibenzyloxy-3-bromo-pyridine (0.5 g, 1.35 mmol) and tert-butyl 6-(4-(4,4,5,5-tetramethyl-1,3,2-dioxaborolan-2-yl)phenyl)-2,6-diazaspiro[3.3]heptane-2-carboxylate (648.75 mg, 1.62 mmol) in dioxane (4.00 mL) and water (2 mL) was added sodium tert-butoxide (389.36 mg, 4.05 mmol) and the reaction mixture was degassed for 15 minutes before cyclopentyl(diphenyl)phosphane; dichloropalladium; iron (197.63 mg, 270.10 μmol) was added. The reaction mixture was stirred at 100° C. for 16 hours. After completion of the reaction as confirmed by LC-MS, the reaction mixture was filtered through celite pad and concentrated under reduced pressure at 50° C. The crude compound was purified by flash column chromatography (silica gel 100-200 mesh, 0-50% ethyl acetate in pet-ether) to afford tert-butyl 6-[4-(2,6-dibenzyloxy-3-pyridyl)phenyl]-2,6-diazaspiro[3.3]heptane-2-carboxylate (0.35 g, 596.33 μmol, 44.16% yield). LC-MS (ES⁺): m/z 564.45 [M+H]⁺.

### Step-4:

To a stirred solution of tert-butyl 6-[4-(2,6-dibenzyloxy-3-pyridyl)phenyl]-2,6-diazaspiro[3.3]heptane-2-carboxylate (0.3 g, 532.21 μmol) in ethanol (20 mL) and THE (20 mL) was added 10% palladium on carbon wet (0.3 g, 2.82 mmol) under nitrogen atmosphere. Then the reaction mixture was stirred at room temperature for 16 hours under a hydrogen balloon. After completion of the reaction as confirmed by LC-MS, the reaction mixture was filtered through a pad of celite and concentrated under reduced pressure at 45° C. The crude compound was purified by flash column chromatography (silica gel 100-200 mesh, 0-30% ethyl acetate in pet-ether) to afford tert-butyl 6-[4-(2,6-dioxo-3-piperidyl)phenyl]-2,6-diazaspiro[3.3]heptane-2-carboxylate (0.15 g, 350.35 μmol, 65.83% yield). LC-MS (ES⁺): m/z 386.36 [M+H]⁺.

### Step-5:

To a stirred solution of tert-butyl 6-[4-(2,6-dioxo-3-piperidyl)phenyl]-2,6-diazaspiro[3.3]heptane-2-carboxylate (0.1 g, 259.43 μmol) in DCM (10 mL) was added trifluoroacetic acid (147.90 mg, 1.30 mmol, 99.93 μL) at 0° C. The reaction mixture was stirred at room temperature for 6 hours. After completion of the reaction as confirmed by.LC-MS, the reaction mixture was concentrated under reduced pressure at 40° C. to afford 3-[4-(2,6-diazaspiro[3.3]heptan-2-yl)phenyl]piperidine-2,6-dione TFA salt (0.1 g, 239.18 μmol, 92.19% yield). LC-MS (ES⁺): m/z 286.32 [M+H]⁺.

### Synthesis of 1-[1-methyl-6-(4-piperidyl) indazol-3-yl]hexahydropyrimidine-2,4-dione

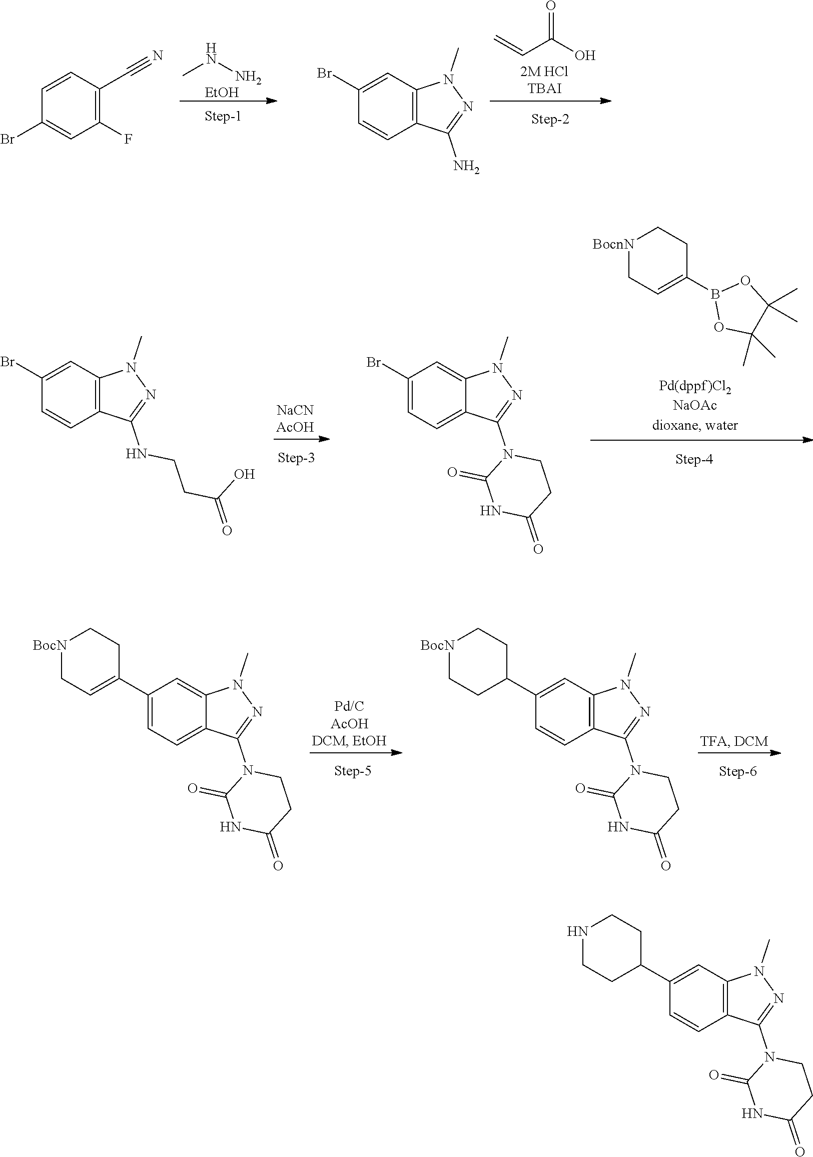

### Step-1:

To a stirred solution of 4-bromo-2-fluorobenzonitrile (25 g, 125.00 mmol) in ethanol (500 mL) was added methyl hydrazine (85% aqueous solution) (51.83 g, 1.12 mol) at room temperature. The reaction mixture was heated at 125° C. in autoclave (1000 ml) for 7 hours. The reaction mixture was cooled to room temperature and poured into ice cold water (2000 ml) and stirred well for 30 minutes. The solidified mass was filtered-off, washed with water, and dried well to afford 6-bromo-1-methyl-1H-indazol-3-amine (25 g, 105.05 mmol, 84.05% yield) as an off-white solid. LC-MS (ES⁺): m/z 291.37 [M+H]⁺.

### Step-2:

To the stirred solution of 6-bromo-1-methyl-indazol-3-amine (50 g, 221.17 mmol) in HCl (2 M aqueous solution) (500.00 mL) was added tetrabutylammonium bromide (7.13 g, 22.12 mmol) at room temperature. The reaction mixture was heated to 55° C. (internal temperature) and acrylic acid (23.91 g, 331.75 mmol, 22.77 mL) was added dropwise at this temperature. The reaction was then heated to 100° C. (external) for 12 hours. After the reaction was complete, the reaction mixture was cooled to room temperature and diluted with ice cold water (1000 ml). It was neutralized to pH 6.5 to 7 with 2 M NaHCO₃ solution (1000 ml) with good stirring. The solid precipitation was filtered-off, washed with excess ice cold water, and dried well to afford 3-[(6-bromo-1-methyl-indazol-3-yl)amino]propanoic acid (54 g, 163.30 mmol, 73.84% yield) as an off-white solid. LC-MS (ES⁺): m/z 298.28 [M+H]⁺.

### Step-3:

To a stirred solution of 3-[(6-bromo-1-methyl-indazol-3-yl)amino]propanoic acid (160 g, 536.67 mmol) in acetic acid (1.07 kg, 17.76 mol, 1.02 L) was added sodium cyanate, 95% (46.67 g, 717.88 mmol). The reaction mixture was heated at 100° C. for 12 hours and the progress was monitored by TLC. Upon completion, the reaction was cooled to room temperature and filtered through a Büchner funnel and washed with water(2×500 mL). The product was dried completely to yield 1-(6-bromo-1-methyl-indazol-3-yl)hexahydro pyrimidine-2,4-dione (175 g, 527.69 mmol, 98.33% yield) as an off-white solid. LC-MS (ES⁺): m/z 323.27 [M+H]⁺.

### Step-4:

To a solution of 1-(6-bromo-1-methyl-indazol-3-yl)hexahydropyrimidine-2,4-dione (15 g, 46.42 mmol) and tert-butyl 4-(4,4,5,5-tetramethyl-1,3,2-dioxaborolan-2-yl)-3,6-dihydro-2H-pyridine-1-carboxylate (18.66 g, 60.34 mmol) in 1,4-dioxane (150 mL) and water (30 mL) was added sodium acetate, anhydrous (11.42 g, 139.26 mmol) at room temperature. The reaction mixture was degassed with argon gas for 10 minutes and 1,1′-bis(diphenylphosphino)ferrocene]palladium(II) dichloride (3.40 g, 4.64 mmol) was added. The reaction mixture was degassed with argon for an additional 5 minutes before it was stirred at 90° C. for 16 hours. Subsequently, the reaction mixture was concentrated in vacuo to yield the crude product, which was purified by column chromatography (silica gel 230-400 mesh, 70% ethyl acetate in pet ether) to afford tert-butyl 4-[3-(2,4-dioxohexahydropyrimidin-1-yl)-1-methyl-indazol-6-yl]-3,6-dihydro-2H-pyridine-1-carboxylate (18 g, 34.69 mmol, 74.73% yield) as a brown solid. LC-MS (ES⁺): m/z 426.44 [M+H]⁺.

### Step-5:

A solution of tert-butyl 4-[3-(2,4-dioxohexahydropyrimidin-1-yl)-1-methyl-indazol-6-yl]-3,6-dihydro-2H-pyridine-1-carboxylate(3.6 g, 8.46 mmol) in ethanol(30 ml) and DCM (10 ml) and a catalytically amount of glacial acetic acid (508.09 mg, 8.46 mmol, 3 ml) was added to a Parr Shaker hydrogenator. Palladium on carbon, 10 wt. % (3.08 g, 25.38 mmol) was added to this mixture under inert atmosphere, and the resulting reaction was stirred for 16 hours at room temperature. The reaction progress was monitored by TLC and LC-MS. Upon completion, the reaction was filtered through celite bed and washed with 10% MeOH/DCM. The filtrate was concentrated under reduced pressure to afford tert-butyl 4-[3-(2,4-dioxohexahydropyrimidin-1-yl)-1-methyl-indazol-6-yl]piperidine-1-carboxylate (3.6 g, 8.17 mmol, 96.55% yield). LC-MS (ES⁺): m/z 428.45 [M+H]⁺.

### Step-6:

To a stirred solution of tert-butyl 4-[3-(2,4-dioxohexahydropyrimidin-1-yl)-1-methyl-indazol-6-yl]piperidine-1-carboxylate (2.7 g, 6.32 mmol) in DCM (20 mL) was added TFA (22.20 g, 194.70 mmol, 15 mL) at 0° C. The reaction was stirred for 3 hours, and the reaction progress was monitored by TLC and LC-MS. Upon completion, the reaction mixture was evaporated to obtain the crude product, which was triturated with diethyl ether and concentrated in vacuo to afford 1-[1-methyl-6-(4-piperidyl) indazol-3-yl]hexahydropyrimidine-2,4-dione TFA salt (2.5 g, 4.92 mmol, 77.93% yield) as a brown solid. LC-MS (ES⁺): m/z 328.48 [M+H]⁺.

### Synthesis of 1-[6-(3,3-difluoro-4-piperidyl)-5-fluoro-1-methyl-indazol-3-yl]hexahydropyrimidine-2,4-dione

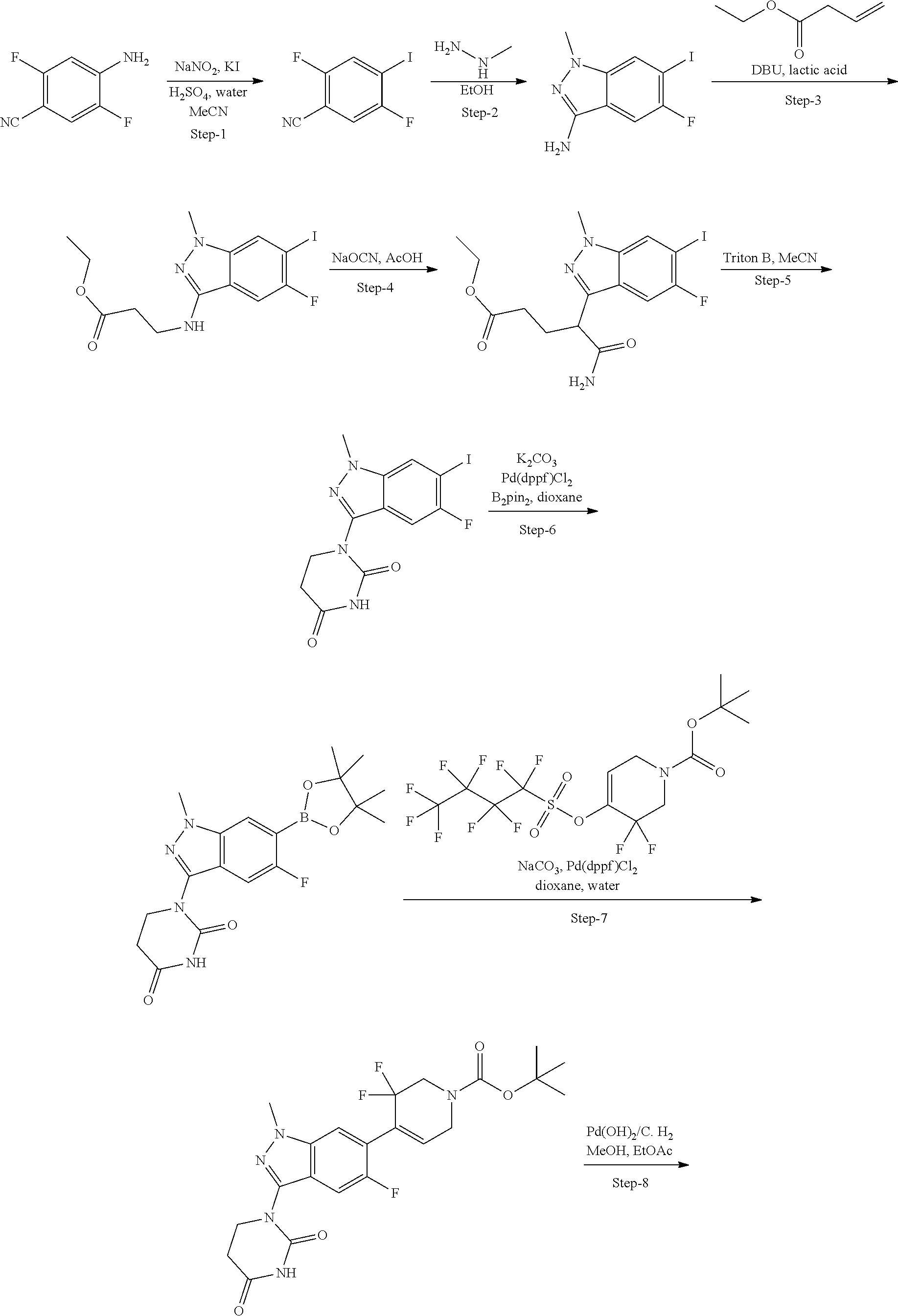

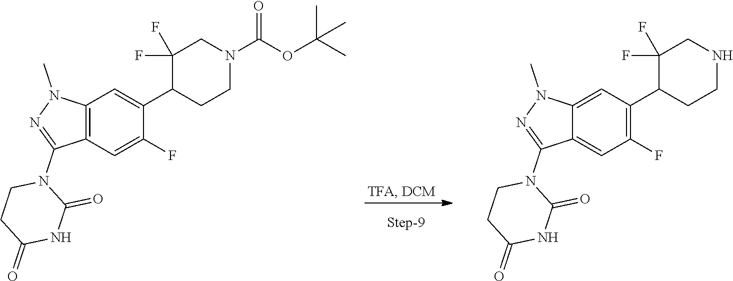 [SMILES: Cn1nc(N2CCC(=O)NC2=O)c2cc(F)c(C3CCN(C(=O)OC(C)(C)C)CC3(F)F)cc21 ; Cn1nc(N2CCC(=O)NC2=O)c2cc(F)c(C3CCNCC3(F)F)cc21]

### Step-1:

In a 5000 mL four-neck round-bottom flask, a solution of 4-amino-2,5-difluoro-benzonitrile (50 g, 324.43 mmol) in ice water (150 mL) and sulfuric acid (150 mL) at 0° C. was added acetonitrile (200 mL). Sodium nitrite (40.29 g, 583.97 mmol, 18.57 mL) in water (120 mL) was added at 0° C. over a period of 1 hour and the resulting mixture was further stirred at this temperature for 1 hour. Potassium iodide (107.71 g, 648.86 mmol) in water (120 mL) was then added at 0° C. and stirred for 80 minutes. The reaction mixture was quenched with sodium thiosulfate at 0° C., stirred for 30 minutes, filtered, washed with water (1000 mL) and dried under reduced pressure. The crude compound was purified by flash column chromatography (silica gel 230-400 mesh. 0-10% ethyl acetate in petroleum ether) to afford 2,5-difluoro-4-iodo-benzonitrile (45 g, 152.83 mmol, 47.11% yield) as an off-white solid. The product was directly taken to the next step. 1H NMR (400 MHz, DMSO-d₆): δ 8.21-8.17 (m, 2H), 8.02-7.99 (m, 2H).

### Step-2:

In a 1000 mL three-neck round bottom flask, a suspension of 2,5-difluoro-4-iodo-benzonitrile (70 g, 264.15 mmol) in ethanol (700 mL) was added methylhydrazine in water 85% (57.27 g, 1.06 mol, 65.83 mL) at ambient temperature. The resulted mixture was stirred at 80° C. for 16 hours. The reaction mixture was cooled to 0° C., diluted with water (1800 mL). After solid was formed, it was stirred for another 30 minutes, filtered, washed with water (1200 mL), petroleum ether (1200 mL) and dried under reduced pressure to give 5-fluoro-6-iodo-1-methyl-indazol-3-amine (45 g, 147.52 mmol, 55.85% yield) as a pale-yellow solid. LC-MS (ES⁺): m/z 292.0 [M+H]⁺.

### Step-3:

In a 1000 mL three-neck round bottom flask, a suspension of 1,8-diazabicyclo[5.4.0]undec-7-ene (36.98 g, 242.92 mmol, 36.26 mL) was added lactic acid 85% aq. soln. (21.88 g, 242.92 mmol, 18.24 mL) at 0° C. The resulted mixture was stirred at ambient temperature for 16 hours. To the above reaction mixture were added 5-fluoro-6-iodo-1-methyl-indazol-3-amine (57 g, 186.86 mmol) and ethyl but-3-enoate (149.30 g, 1.31 mol, 158.83 mL) at ambient temperature. The resulted mixture was stirred at 80° C. for 48 hours. The reaction mixture was cooled to 0° C., quenched with water (500 mL), extracted with ethyl acetate (3×400 mL), washed with brine (300 mL), dried over anhydrous sodium sulfate, filtered, and concentrated under reduced pressure. The crude compound was purified by flash column chromatography (silica gel 230-400 mesh, 20-30% ethyl acetate in petroleum ether) to give ethyl 3-[(5-fluoro-6-iodo-1-methyl-indazol-3-yl)amino]propanoate (55 g, 125.74 mmol, 67.29% yield) as a pale-yellow semi-solid. LC-MS (ES⁺): m/z 392.0 [M+H]⁺.

### Step-4:

Into a 1000 mL three-neck round bottom flask was containing a well-stirred suspension of ethyl 3-[(5-fluoro-6-iodo-1-methyl-indazol-3-yl)amino]propanoate (55 g, 125.74 mmol) in acetic acid (550 mL) was added sodium cyanate (16.35 g, 251.48 mmol) at ambient temperature. The resulted mixture was stirred at 80° C. for 16 hours. The reaction mixture was concentrated under reduced pressure, cooled to 0° C., quenched with 10% sodium bicarbonate (1300 mL), and extracted with dichloromethane (600 mL). The organic layer was washed with 10% sodium bicarbonate (500 mL), brine (400 mL), dried over anhydrous sodium sulfate, filtered, and concentrated under reduced pressure. The crude compound was purified by flash column chromatography (silica gel 230-400 mesh, 90-100% ethyl acetate in petroleum ether) to afford ethyl 3-[carbamoyl-(5-fluoro-6-iodo-1-methyl-indazol-3-yl)amino]propanoate (40 g, 89.56 mmol, 71.23% yield) as an off-white solid. LC-MS (ES⁺): m/z 435.0 [M+H]⁺.

### Step-5:

Into a 1000 mL single neck round bottom flask was containing a well-stirred solution of ethyl 3-[carbamoyl-(5-fluoro-6-iodo-1-methyl-indazol-3-yl)amino]propanoate (67.4 g, 150.91 mmol) in acetonitrile (330 mL) was added benzyltrimethylammonium hydroxide, 40% in methanol (18.93 g, 45.27 mmol, 20.58 mL) at ambient temperature. The resulted mixture was stirred at ambient temperature for 1 hour. The reaction mixture was diluted with petroleum ether (330 mL), and the resulting solid was filtered, washed with petroleum ether (500 mL), dried under reduced pressure to afford 1-(5-fluoro-6-iodo-1-methyl-indazol-3-yl)hexahydropyrimidine-2,4-dione (50.67 g, 128.25 mmol, 84.98% yield) as an off-white solid. LC-MS (ES⁺): m/z 389.0 [M+H]⁺.

### Step-6:

In a 250 mL sealed tube containing a stirred solution of 1-(5-fluoro-6-iodo-1-methyl-indazol-3-yl)hexahydropyrimidine-2,4-dione (2.5 g, 6.44 mmol) in dioxane (20 mL) were added bis(pinacolato)diboron (7.07 g, 27.85 mmol) and potassium acetate(5.47 g, 55.70 mmol). The reaction mixture was degassed with nitrogen for 10 minutes before Pd(dppf)Cl₂ CH₂Cl₂ (1.52 g, 1.86 mmol) was added to the reaction mixture and the reaction mixture was degassed with nitrogen for another 10 minutes. The reaction mixture was heated at 100° C. for 16 hours while the reaction progress was monitored by TLC and UPLC. The reaction mixture was cooled to room temperature, diluted with water, and then extracted with ethyl acetate. The combined organic layers were washed with brine, dried over anhydrous sodium sulfate. The organic layer was concentrated under reduced pressure to get the crude product, which was purified by column chromatography (Biotage Isolera, desired product eluted at 60% to 65% ethyl acetate in petroleum ether). Compound 1-[5-fluoro-1-methyl-6-(4,4,5,5-tetramethyl-1,3,2-dioxaborolan-2-yl) indazol-3-yl]hexahydropyrimidine-2,4-dione (2 g, 3.13 mmol, 48.65% yield) was obtained as an off-white solid. LC-MS (ES⁺): m/z 389.3 [M+H]⁺.

### Step-7:

Into 250 mL sealed tube containing well stirred solution of 1-[5-fluoro-1-methyl-6-(4,4,5,5-tetramethyl-1,3,2-dioxaborolan-2-yl) indazol-3-yl]hexahydropyrimidine-2,4-dione (1.0 g, 2.58 mmol) and tert-butyl 3,3-difluoro-4-(1,1,2,2,3,3,4,4,4-nonafluorobutylsulfonyl oxy)-2,6-dihydropyridine-1-carboxylate (1.47 g, 2.83 mmol) in 1′4-dioxane (16 mL) and water (4 mL) was added sodium carbonate (819.09 mg, 7.73 mmol). The mixture was purged with nitrogen gas for 10 minutes. Then [1,1′-Bis(diphenylphosphino)ferrocene]dichloropalladium(II) (1:1) (210.20 mg, 257.60 μmol) was added and the reaction mixture was purged with nitrogen for another 2 minutes. The resulting mixture was stirred at 60° C. for 2 hours. The progress of reaction was monitored by TLC and LC-MS. After completion of the reaction, the mixture was cooled to room temperature, the reaction mass was diluted with ethyl acetate (100 mL) and water (50 mL). The organic layers were separated, dried over anhydrous Na₂SO₄, filtered and concentrated under reduced pressure to get the crude. The obtained crude product was purified by flash column chromatography (silica gel 100-200 mesh, 50-80% ethyl acetate in petroleum ether) to obtain tert-butyl 4-[3-(2,4-dioxohexahydropyrimidin-1-yl)-5-fluoro-1-methyl-indazol-6-yl]-3,3-difluoro-2,6-dihydropyridine-1-carboxylate (1.0 g, 1.84 mmol, 71.25% yield) as a pale brown color solid. LC-MS (ES⁺): m/z 480.5 [M+H]⁺.

### Step-8:

Into a 25 mL flask containing a well stirred solution of tert-butyl 4-[3-(2,4-dioxohexahydropyrimidin-1-yl)-5-fluoro-1-methyl-indazol-6-yl]-3,3-difluoro-2,6-dihydropyridine-1-carboxylate (1.4 g, 2.92 mmol) in anhydrous methanol (5 mL) was added palladium hydroxide on carbon, 20 wt. %, 50% water (820.14 mg, 5.84 mmol) at room temperature. The contents were stirred at room temperature for 16 hours under hydrogen gas. Progress of the reaction was monitored by UPLC and TLC. After complete conversion of starting material, the reaction mixture was filtered through a pad of celite under nitrogen atmosphere and the filtrate was concentrated under reduced pressure. The crude product was purified by column chromatography (Biotage® Isolera, desired product eluted at 10% to 15% methanol in DCM) to afford tert-butyl 4-[3-(2,4-dioxohexahydropyrimidin-1-yl)-5-fluoro-1-methyl-indazol-6-yl]-3,3-difluoro-piperidine-1-carboxylate (0.6 g, 1.06 mmol, 36.15% yield) as an off white solid. LC-MS (ES⁺): m/z 426.2 [M-tBu+H]⁺.

### Step-9:

Into a 50 mL single neck round bottom flask containing a solution of tert-butyl 4-[3-(2,4-dioxohexahydropyrimidin-1-yl)-5-fluoro-1-methyl-indazol-6-yl]-3,3-difluoro-piperidine-1-carboxylate (70 mg, 145.39 μmol) in DCM (10 mL) was added Hydrogen chloride, 4M in 1,4-dioxane, 99% (800.00 mg, 21.94 mmol, 1 mL) at 0 C, the resulting reaction mixture was stirred at room temperature for 1 hr. The progress of reaction was monitored by TLC and UPLC. After completion of the reaction, the reaction mixture was concentrated under vacuum and washed with diethyl ether to give a product 1-[6-(3,3-difluoro-4-piperidyl)-5-fluoro-1-methyl-indazol-3-yl]hexahydropyrimidine-2,4-dione HCl salt (60 mg, 119.77 μmol, 82.38% yield) as an off-white solid. LC-MS (ES⁺): m/z 382.2 [M+H]⁺.

### Synthesis of 1-(1-methyl-6-piperazin-1-yl-indazol-3-yl)hexahydropyrimidine-2,4-dione

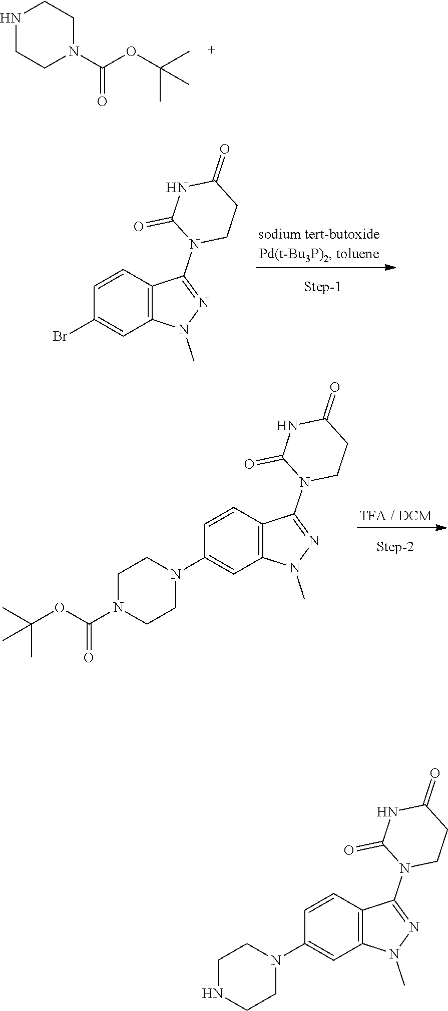 [SMILES: CC(C)(C)OC(=O)N1CCNCC1 ; Cn1nc(N2CCC(=O)NC2=O)c2ccc(Br)cc21 ; Cn1nc(N2CCC(=O)NC2=O)c2ccc(N3CCN(C(=O)OC(C)(C)C)CC3)cc21 ; Cn1nc(N2CCC(=O)NC2=O)c2ccc(N3CCNCC3)cc21]

### Step-1:

In a 100 mL round bottom flask, to a stirred solution of 1-(6-bromo-1-methyl-indazol-3-yl)hexahydropyrimidine-2,4-dione (0.5 g, 1.55 mmol) in toluene (10 mL) was added tert-butyl piperazine-1-carboxylate (288.18 mg, 1.55 mmol) and sodium tert-butoxide (297.40 mg, 3.09 mmol) at room temperature. The reaction mixture was degassed with argon for 10 minutes, then Pd(t-Bu₃P)₂ (79.07 mg, 154.73 μmol) was added, then again degassed for 5 minutes. It was stirred at 110° C. for 16 hours, while the progress of reaction was monitored by LC-MS. The reaction mixture was evaporated to give a residue, which was poured into water (20 mL), and the resulting solution was extracted with DCM (2×50 mL). The combined organic layers were washed with brine (50 mL), dried over anhydrous Na₂SO₄, and evaporated to afford the crude product, which was triturated with diethyl ether (30 mL) to afford tert-butyl 4-[3-(2,4-dioxohexahydropyrimidin-1-yl)-1-methyl-indazol-6-yl]piperazine-1-carboxylate (0.310 g, 614.96 μmol, 39.74% yield) as a pale yellow solid. LC-MS (ES⁺): m/z 429.50 [M+H]⁺.

### Step-2:

To a stirred solution of tert-butyl 4-[3-(2,4-dioxohexahydropyrimidin-1-yl)-1-methyl-indazol-6-yl]piperazine-1-carboxylate (0.3 g, 700.14 μmol) in DCM (10 mL) was added TFA (1.48 g, 12.98 mmol, 1 mL) at 0° C. and stirring was continued for 6 hours at room temperature. The reaction progress was monitored by LC-MS. After the completion of reaction, the solvent was evaporated under vacuum to obtain the crude product. The crude was triturated in diethyl ether (20 mL) and the solid was filtered and dried to afford 1-(1-methyl-6-piperazin-1-yl-indazol-3-yl)hexahydropyrimidine-2,4-dione (0.280 g, 591.28 μmol, 84.45% yield) as a pale-yellow solid. LC-MS (ES⁺): m/z 329.30 [M+H]⁺.

### Synthesis of 1-(5-fluoro-1-methyl-6-piperazin-1-yl-indazol-3-yl)hexahydropyrimidine-2,4-dione

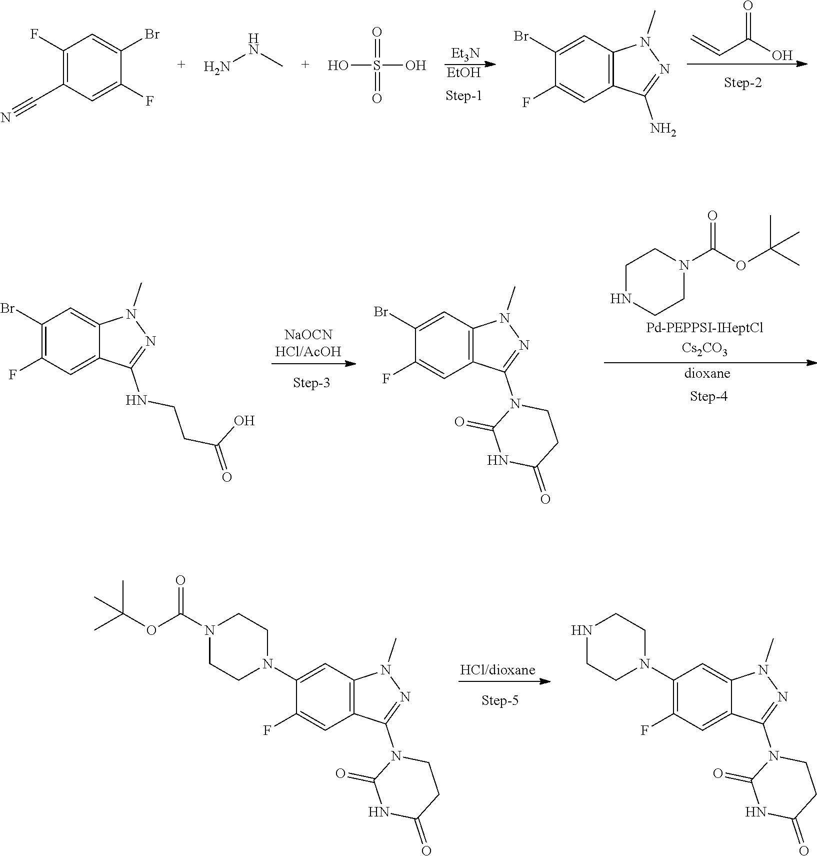

### Step-1:

To a solution of 4-bromo-2,5-difluoro-benzonitrile (10 g, 45.87 mmol) in EtOH (30 mL) was added methylhydrazine sulfuric acid (19.84 g, 137.62 mmol) and Et₃N (18.57 g, 183.49 mmol, 25.61 mL). The mixture was stirred at 80° C. for 12 hours. LC-MS showed starting material was consumed completely and one main peak with desired mass was detected. The mixture was cooled down to 30° C., water (300 mL) was added. The mixture was filtered and the filter cake was washed with water (5 mL×2), and then concentrated at 40° C. under vacuum to afford 6-bromo-5-fluoro-1-methyl-indazol-3-amine (6.5 g, 25.30 mmol, 55.16% yield, 95% purity) as a yellow solid. ¹H-NMR (400 MHz, DMSO-d₆) δ=7.46 (d, J=8.4 Hz, 1H), 7.06-7.04 (m, 1H), 5.68 (s, 2H), 3.83 (d, J=0.8 Hz, 3H). LC-MS (ES⁺): m/z 245 [M+H]⁺.

### Step-2:

To a solution of 6-bromo-5-fluoro-1-methyl-indazol-3-amine (22 g, 90.14 mmol) and acrylic acid (9.74 g, 135.21 mmol, 9.28 mL) in 2 M aq. HCl (220 mL) was added tetrabutylammonium bromide (2.91 g, 9.01 mmol). The mixture was stirred at 100° C. for 12 hours. LC-MS showed complete consumption and one main peak with desired mass was detected. All the reaction mixture was basified with a saturated solution of NaHCO₃ until pH=8. The solution was acidified with acetic acid to pH=5. A white solid was precipitated, filtered, and washed with water (250 ml), then dried under reduced pressure to afford 3-[(6-bromo-5-fluoro-1-methyl-indazol-3-yl)amino] propanoic acid (28 g, 88.57 mmol, 98.26% yield) as a white solid. LC-MS (ES⁺): m/z 318.2 [M+H]⁺.

### Step-3:

To a solution of 3-[(6-bromo-5-fluoro-1-methyl-indazol-3-yl)amino]propanoic acid (26 g, 82.25 mmol) in AcOH (260 mL) was added NaOCN (11.36 g, 164.49 mmol). The mixture was stirred at 60° C. for 16 hours. To the mixture was added HCl (260 mL). The mixture was stirred at 60° C. for another 3 hours. LCMS showed starting material was consumed completely and one main peak with desired mass was detected. The reaction mixture was cooled down to room temperature and stirred for 1 hour, filtered and washed with water (250 mL). The cake was dried under vacuum to afford 1-(6-bromo-5-fluoro-1-methyl-indazol-3-yl)hexahydropyrimidine-2,4-dione (18 g, 47.63 mmol, 57.91% yield, 90.26% purity) as a white solid. ¹H-NMR (400 MHz, DMSO-d₆) δ=10.59 (s, 1H), 8.16 (d, J=5.6 Hz, 1H), 7.62 (d, J=9.2 Hz, 1H), 4.00 (s, 3H), 3.93-3.90 (m, 2H), 2.77-2.73 (m, 2H).

### Step-4:

To a solution of 1-(6-bromo-5-fluoro-1-methyl-indazol-3-yl)hexahydropyrimidine-2,4-dione (5 g, 14.66 mmol) and tert-butyl piperazine-1-carboxylate (8.19 g, 43.97 mmol) in dioxane (50 mL) was added Pd-PEPPSI-IHeptCl (427.40 mg, 439.71 μmol) and Cs₂CO₃ (14.33 g, 43.97 mmol) at 25° C. under N₂ atmosphere. The reaction mixture was stirred at 100° C. under N₂ for 16 hours. LC-MS showed complete consumption and desired mass detected. The reaction mixture was diluted with water (200 mL), extract with EtOAc (100 mL×3). The combined organic layer was dried over Na₂SO₄, filtered, and concentrated to a residue which was triturated by EtOAc:MTBE (1:5). The suspension was filtered and dried to afford tert-butyl 4-[3-(2,4-dioxohexahydropyrimidin-1-yl)-5-fluoro-1-methyl-indazol-6-yl]piperazine-1-carboxylate (3.4 g, 6.85 mmol, 46.76% yield) as a gray solid. ¹H-NMR (400 MHz, DMSO-d₆) δ=10.53 (s, 1H), 7.38 (d, J=12.8 Hz, 1H), 7.16 (d, J=6.8 Hz, 1H), 3.94 (s, 3H), 3.89-3.87 (m, 2H), 3.52 (br s, 4H), 3.06-2.98 (m, 4H), 2.75-2.73 (m, 2H), 1.43 (s, 9H).

### Step-5:

A solution of tert-butyl 4-[3-(2,4-dioxohexahydropyrimidin-1-yl)-5-fluoro-1-methyl-indazol-6-yl]piperazine-1-carboxylate (2.4 g, 5.38 mmol) in 4M HCl/dioxane (30 mL) was stirred at 25° C. for 2 hours. TLC showed reactant was consumed and a new spot was formed. The reaction mixture was concentrated to a residue which was triturated with MTBE (200 mL), filtered, and the filter cake was dried under vacuum to afford 1-(5-fluoro-1-methyl-6-piperazin-1-yl-indazol-3-yl)hexahydropyrimidine-2,4-dione (2 g, 4.70 mmol, 87.47% yield) as a gray solid. ¹H-NMR (400 MHz, DMSO-d₆) δ=10.54 (s, 1H), 9.22 (br s, 2H), 7.41 (d, J=12.4 Hz, 1H), 7.23 (d, J=7.2 Hz, 1H), 3.97 (s, 3H), 3.91-3.88 (m, 2H), 3.31 (br s, 8H), 2.76-2.72 (m, 2H).

### Synthesis of 3-[3-methyl-2-oxo-5-(4-piperidyl)benzimidazol-1-yl]piperidine-2,6-dione

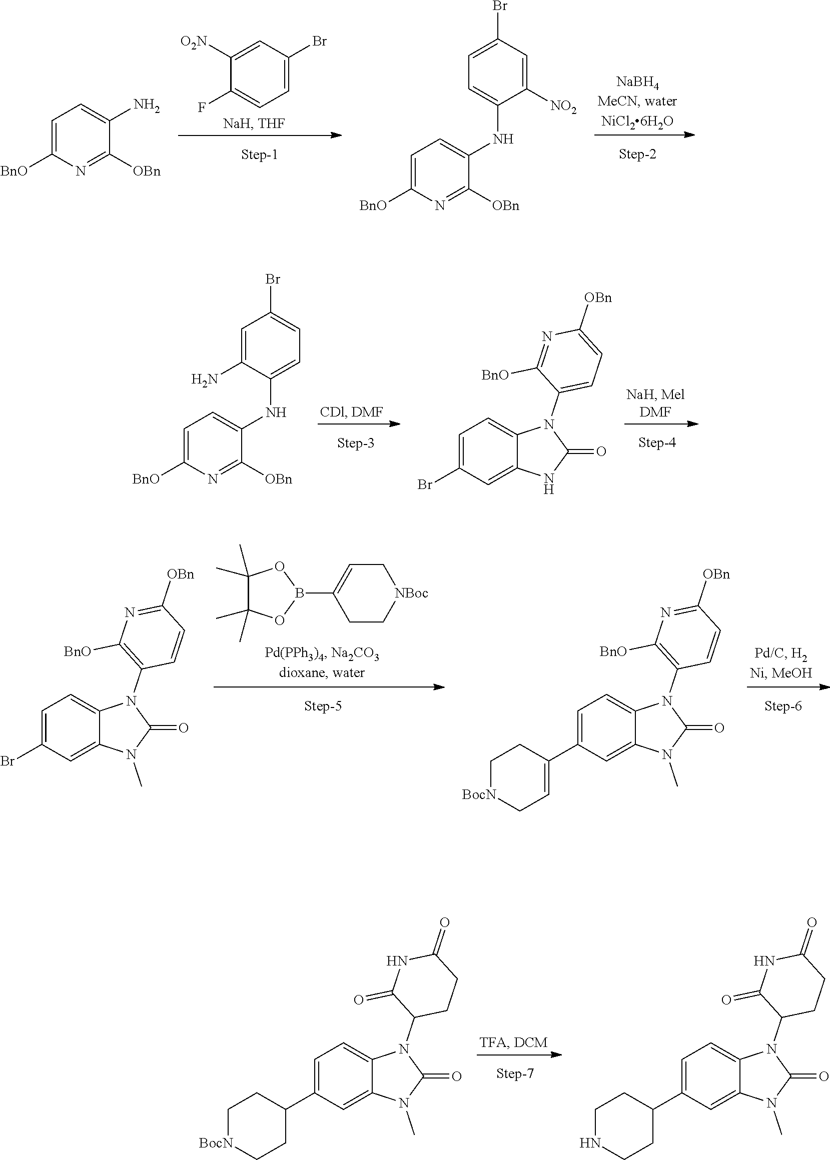

### Step-1:

To sodium hydride (in oil dispersion) 60% dispersion in mineral oil (53.51 g, 2.33 mol) was added THE (2300 mL) and the suspension was cooled to 5-10° C. A solution of 2,6-dibenzyloxypyridin-3-amine (230 g, 750.76 mmol) in THF (1400 mL) was added at 5-10° C. over 20 minutes with exothermicity observed. The temperature was maintained for 30 minutes. To this solution was added 4-bromo-1-fluoro-2-nitrobenzene, 98% (247.75 g, 1.13 mol, 138.41 mL) in THE (1600 mL) at 5-10° C. over 20 minutes. The solution was warmed to room temperature and the temperature was maintained for 16 hours. TLC (20% EtOAc in pet ether) confirmed the formation of product. The reaction mass was quenched with 10% water in THE (5 V) at below 10° C., with observed exothermicity. Saturated NaCl solution (10 V) was added at below 15° C. and warmed to room temperature. The layers were separated, and the organic layer was concentrated under vacuum. The aqueous layer was taken and extracted with DCM (15 V) and kept aside. The organic layer was combined with crude and washed with water (5 V) and concentrated completely under vacuum at 45° C. The crude was charged into DCM (2.5 V) at 45° C. and maintained for 15 min until dissolution, then added pet ether (10 V) at 45° C. and maintained for 1 hr at 45° C. Cooled to RT and maintained for 30 min. Filtered and washed with pet ether (2*3 V) to afford 2,6-dibenzyloxy-N-(4-bromo-2-nitro-phenyl)pyridin-3-amine (400 g, 686 mmol, 91% yield). LC-MS (ES⁺): m/z 506.32 [M+H]⁺.

### Step-2:

A solution of 2,6-dibenzyloxy-N-(4-bromo-2-nitro-phenyl)pyridin-3-amine (50 g, 98.75 mmol) in ACN (450 mL) and water (50 mL) was cooled to 0-5° C. and sodium borohydride (7.47 g, 197.49 mmol, 6.98 mL) was added portionwise for 60 hours, during which room temperature was maintained for 4 hours. TLC was used to monitor the progress of the reaction. Sodium borohydride (7.47 g, 197.49 mmol, 6.98 mL) was added at 0-5° C. and temperature maintained for 2 hours. Then the reaction was quenched with 10% NH₄Cl solution (5 V), water added (5 V), followed by DCM (10 V), then stirred at RT for 15 min. The aqueous layer was extracted with DCM (10 V) and the combined organic layers were washed with water (10 V) and concentrated completely under vacuum at 40° C. Pet ether was used to strip the residue (3 V), then charged into 10% EtOAc in pet ether (5 V) into a crude residue and heated to 45° C. The temperature was maintained at 45° C. for 30 min, cooled to RT, and maintained for 30 min. The pure product was filtered and washed with pet ether (3 V). LC-MS (ES⁺): m/z 476.33 [M+H]⁺.

### Step-3:

To the stirred solution of 4-bromo-N1-(2,6-dibenzyloxy-3-pyridyl)benzene-1,2-diamine (200 g, 419.85 mmol) in DMF (800 mL) was added di(imidazol-1-yl)methanone (177.00 g, 1.09 mol) at 25-35° C. with observed exothermicity. CDI was charged as a single lot. Initial temperature 25° C. was monitored with the final temperature of 35° C. noted at 15 minutes. The reaction was stirred for 14 hours at room temperature. TLC showed the consumption of starting material. The reaction was charged into water (420 mL) at room temperature. Precipitation was formed (Note: Slow addition required a minimum of 1 h for bulk scale) and the mixture was stirred for 3 hours. The solid was filtered and washed with water and pet ether (2×35 ml). The product was dried under vacuum for 7 hours at 50° C. to afford 6-bromo-3-(2,6-dibenzyloxy-3-pyridyl)-1H-benzimidazol-2-one (200 g, 391.43 mmol, 93.23% yield). LC-MS (ES⁺): m/z 502.1 [M+H]⁺.

### Step-4:

To a stirred solution of 6-bromo-3-(2,6-dibenzyloxy-3-pyridyl)-1H-benzimidazol-2-one (108 g, 214.99 mmol) in DMF (1000 mL) was added sodium hydride (60% dispersion in mineral oil) (14.83 g, 644.96 mmol) portionwise at 0-28° C. The reaction mixture was stirred for 1 hour, followed by dropwise addition of methyl iodide (stored over copper) (31.16 g, 214.99 mmol, 13.37 mL) over half an hour. Progress of the reaction was monitored by TLC and LC-MS-. The reaction mixture was diluted with ice cold water, and the resulting solid was obtained, filtered, and dried over vacuum. The solid was extracted with ethyl acetate, then washed with brine, dried over sodium sulfate, and concentrated to dryness. The crude compound was washed with pentane to afford the product 5-bromo-1-(2,6-dibenzyloxy-3-pyridyl)-3-methyl-benzimidazol-2-one (95 g, 183.81 mmol, 85.50% yield, 99.91% purity) as a light brown solid. LC-MS (ES⁺): m/z 516.14 [M+H]⁺.

### Step-5:

To a solution of 5-bromo-1-(2,6-dibenzyloxy-3-pyridyl)-3-methyl-benzimidazol-2-one (20 g, 38.73 mmol) in 1,4-dioxane (160 mL) and water (40 mL) was added sodium carbonate (12.32 g, 116.19 mmol) and tert-butyl 4-(4,4,5,5-tetramethyl-1,3,2-dioxaborolan-2-yl)-3,6-dihydro-2H-pyridine-1-carboxylate (15.57 g, 50.35 mmol). The reaction was purged with nitrogen for 20 minutes, then charged with palladium (0) tetrakis(triphenylphosphine) (2.24 g, 1.94 mmol) and heated to 90-100° C. for 5 hours. TLC confirmed the formation of product. The reaction was cooled to room temperature and filtered through a celite bed and washed with EtOAc. The filtrate was taken and distilled completely under vacuum at 45° C. The crude product was dissolved in EtOAc (15 V) and separated with water (10 V). The organic layer was washed with water (5 V), brine (5 V), then dried over anhydrous Na₂SO₄. The organic layer was concentrated in vacuo at 45° C. then purified by column chromatography (100-200 mesh silica gel, 0-30% ethyl acetate in pet ether) to afford tert-butyl 4-[1-(2,6-dibenzyloxy-3-pyridyl)-3-methyl-2-oxo-benzimidazol-5-yl]-3,6-dihydro-2H-pyridine-1-carboxylate (21 g, 33.06 mmol, 99% yield). LC-MS (ES⁺): m/z 619.41 [M+H]⁺.

### Step-6:

To a solution of tert-butyl 4-[1-(2,6-dibenzyloxy-3-pyridyl)-3-methyl-2-oxo-benzimidazol-5-yl]-3,6-dihydro-2H-pyridine-1-carboxylate (40 g, 64.65 mmol) in methanol (1600 mL) was added palladium, 10% on carbon, type 487, dry (12.00 g, 112.76 mmol) and nickel (12.00 g, 204.45 mmol). Hydrogen gas (10 kg) was applied and the reaction was maintained at 60-65° C. for 16 hours. The reaction mass was cooled to room temperature then filtered and washed with DCM and MeOH. The filtrate was taken and distilled completely under vacuum at 45° C. To the crude residue was added IPA (3 V) and heated to 60° C. for 15 minutes. Pet ether (3 V) was added and the mixture cooled to room temperature, and stirred at this temperature for 1 hour. The solid was filtered and washed with pet ether to afford tert-butyl 4-[1-(2,6-dioxo-3-piperidyl)-3-methyl-2-oxo-benzimidazol-5-yl]piperidine-1-carboxylate (21 g, 44 mmol, 69% yield). LC-MS (ES⁻): m/z 441.18 [M−H]⁻.

### Step-7:

To a solution of tert-butyl 4-[1-(2,6-dioxo-3-piperidyl)-3-methyl-2-oxo-benzimidazol-5-yl]piperidine-1-carboxylate (7.5 g, 16.95 mmol) in DCM (75 mL) was added trifluoroacetic acid (55.87 g, 490.03 mmol, 37.75 mL) at 0-5° C. slowly and the temperature was maintained for 15 minutes. The reaction was warmed to room temperature and maintained for 3 hours. LCMS complied with the formation of product. DCM and TFA were removed under vacuum at 40° C. and the crude stripped off with toluene (2×5 V) and diethyl ether added with the formation of solid observed. The reaction was decanted after adding diethyl ether (3×5 V), then dried at 45° C. The crude was dissolved in MeOH (10 V), stirred for 10 minutes, and filtered through a sintered funnel and washed with MeOH with slight undissolved particles observed. The distilled filtrate was completely evaporated under vacuum at 45° C. to afford 3-[3-methyl-2-oxo-5-(4-piperidyl)benzimidazol-1-yl]piperidine-2,6-dione (7.72 g, 16.5 mmol, 97% yield). LC-MS (ES⁻): m/z 343.35 [M−H]⁻.

### Synthesis of 3-[5-(3,3-difluoro-4-piperidyl)-3-methyl-2-oxo-benzimidazol-1-yl]piperidine-2,6-dione

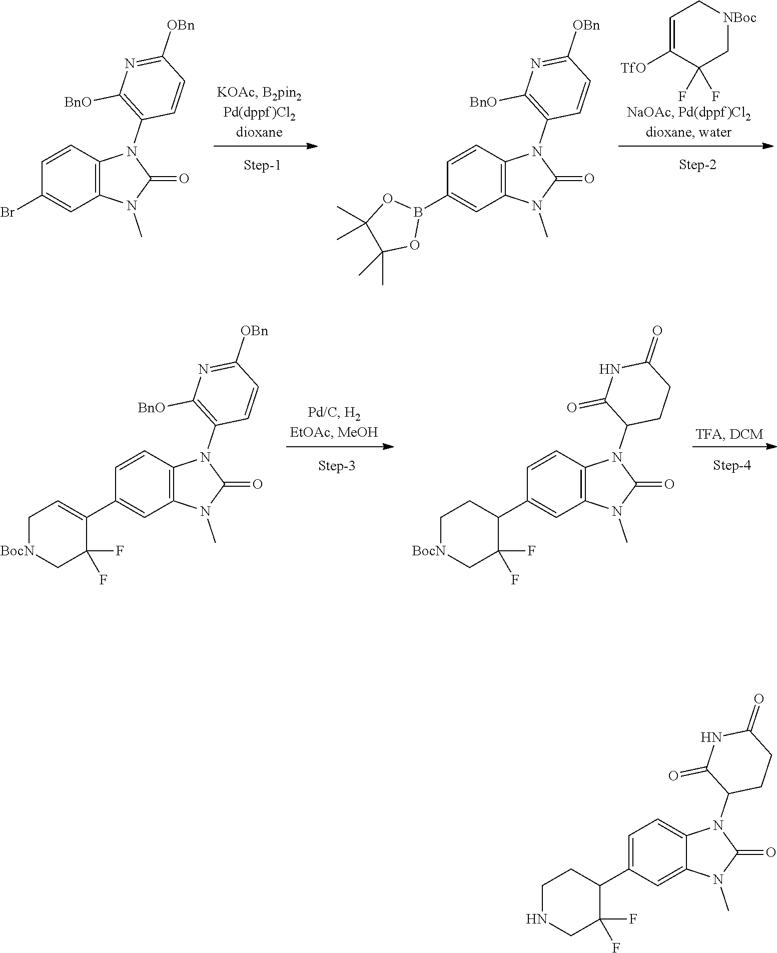 [SMILES: Cn1c(=O)n(-c2ccc(OCc3ccccc3)nc2OCc2ccccc2)c2ccc(Br)cc21 ; Cn1c(=O)n(-c2ccc(OCc3ccccc3)nc2OCc2ccccc2)c2ccc(B3OC(C)(C)C(C)(C)O3)cc21 ; CC(C)(C)OC(=O)N1CC=C(OS(=O)(=O)C(F)(F)F)C(F)(F)C1 ; Cn1c(=O)n(-c2ccc(OCc3ccccc3)nc2OCc2ccccc2)c2ccc(C3=CCN(C(=O)OC(C)(C)C)CC3(F)F)cc21 ; Cn1c(=O)n(C2CCC(=O)NC2=O)c2ccc(C3CCN(C(=O)OC(C)(C)C)CC3(F)F)cc21 ; Cn1c(=O)n(C2CCC(=O)NC2=O)c2ccc(C3CCNCC3(F)F)cc21]

### Step-1:

In a 100 mL sealed tube, to a solution of 5-bromo-1-(2,6-dibenzyloxy-3-pyridyl)-3-methyl-benzimidazol-2-one (1.5 g, 2.90 mmol) in 1,4 dioxane (1 mL) were added 4,4,5,5-tetramethyl-2-(4,4,5,5-tetramethyl-1,3,2-dioxaborolan-2-yl)-1,3,2-dioxaborolane (1.11 g, 4.36 mmol) and potassium acetate (855.25 mg, 8.71 mmol) at room temperature under argon gas. The reaction mixture was degassed with argon for 20 minutes before cyclopentyl(diphenyl)phosphane;dichloromethane;dichloropalladium;iron (118.61 mg, 145.24 μmol) was added and the reaction heated at 100° C. for 6 hours while monitoring with TLC and LC-MS. After completion of the reaction, the solvent was removed under reduced pressure and extracted using EtOAc (50 mL×3) and water (50 mL). The combined organic layers were washed with brine solution (50 mL), dried over anhydrous sodium sulfate, filtered, and concentrated under reduced pressure. The residue was purified by column chromatography (100-200 mesh silica gel, 0-30% EtOAc in pet-ether) to afford 1-(2,6-dibenzyloxy-3-pyridyl)-3-methyl-5-(4,4,5,5-tetramethyl-1,3,2-dioxaborolan-2-yl)benzimidazol-2-one (1.3 g, 2.17 mmol, 74.66% yield) as a pale yellow solid. LC-MS (ES⁺): m/z 264.36 [M+H]⁺.

### Step-2:

In a sealed tube, to a solution of 1-(2,6-dibenzyloxy-3-pyridyl)-3-methyl-5-(4,4,5,5-tetramethyl-1,3,2-dioxaborolan-2-yl)benzimidazol-2-one (1.2 g, 2.13 mmol) in dioxane (12 mL) and water (4 mL) were added tert-butyl 3,3-difluoro-4-(trifluoromethylsulfonyloxy)-2,6-dihydropyridine-1-carboxylate (938.67 mg, 2.56 mmol) and sodium acetate (524.13 mg, 6.39 mmol) at room temperature under argon gas. The reaction mixture was degased with argon for 20 minutes. After degassing, cyclopentyl(diphenyl)phosphane; dichloropalladium; iron (155.83 mg, 212.97 μmol) was added and the reaction was heated at 100° C. for 16 hours while monitoring with TLC and LC-MS. The catalyst was filtered off through celite and washed with ethyl acetate (20 mL×3). The filtrate was washed with water (20 mL) and brine solution (20 mL). The combined organic layers were dried over anhydrous sodium sulfate, filtered, and concentrated under reduced pressure. The crude was purified by column chromatography (230-400 mesh silica gel, 0-60% EtOAc in pet-ether) to afford tert-butyl 4-[1-(2,6-dibenzyloxy-3-pyridyl)-3-methyl-2-oxo-benzimidazol-5-yl]-3,3-difluoro-2,6-dihydropyridine-1-carboxylate (1.3 g, 1.97 mmol, 92.30% yield) as a colorless thick liquid. LC-MS (ES⁺): m/z 655.34 [M+H]⁺.

### Step-3:

To a stirred solution of tert-butyl 4-[1-(2,6-dibenzyloxy-3-pyridyl)-3-methyl-2-oxo-benzimidazol-5-yl]-3,3-difluoro-2,6-dihydropyridine-1-carboxylate (1.3 g, 1.99 mmol) in ethyl acetate (20 mL) and ethanol (5 mL) was added palladium, 10% on carbon, type 487, dry (975.00 mg, 9.16 mmol) and dioxoplatinum (433.33 mg, 1.91 mmol). The reaction was stirred for 6 hours at room temperature under hydrogen atmosphere. The reaction progress was monitored by TLC and LC-MS. The reaction mixture was filtered through celite using ethyl acetate (50 mL) and the filtrate was concentrated under reduced pressure. The crude product was triturated in diethyl ether (30 mL), then decanted and dried under reduced pressure to obtain the product tert-butyl 4-[1-(2,6-dioxo-3-piperidyl)-3-methyl-2-oxo-benzimidazol-5-yl]-3,3-difluoro-piperidine-1-carboxylate (0.460 g, 922.91 μmol, 46.48% yield) as a white color solid. LC-MS (ES⁺): m/z 479.35 [M+H]⁺.

### Step-4:

To a stirred solution of tert-butyl 4-[1-(2,6-dioxo-3-piperidyl)-3-methyl-2-oxo-benzimidazol-5-yl]-3,3-difluoro-piperidine-1-carboxylate (0.45 g, 940.46 μmol.) in DCM (10 mL) at 0° C. was added TFA (3.33 g, 29.20 mmol, 2.25 mL) over 5 minutes. The reaction mixture was stirred at 25° C. for 4 hours while the reaction progress was monitored by TLC. After completion of the reaction, the reaction mixture was concentrated and co-distilled with toluene (10 ml) and diethyl ether (2×50 ml) to afford the product 3-[5-(3,3-difluoro-4-piperidyl)-3-methyl-2-oxo-benzimidazol-1-yl]piperidine-2,6-dione (0.3 g, 536.15 μmol, 57.01% yield) as a yellow solid. LC-MS (ES⁺): m/z 379.53 [M+H]⁺.

### Synthesis of 3-[3-methyl-2-oxo-4-(4-piperidyl)benzimidazol-1-yl]piperidine-2,6-dione

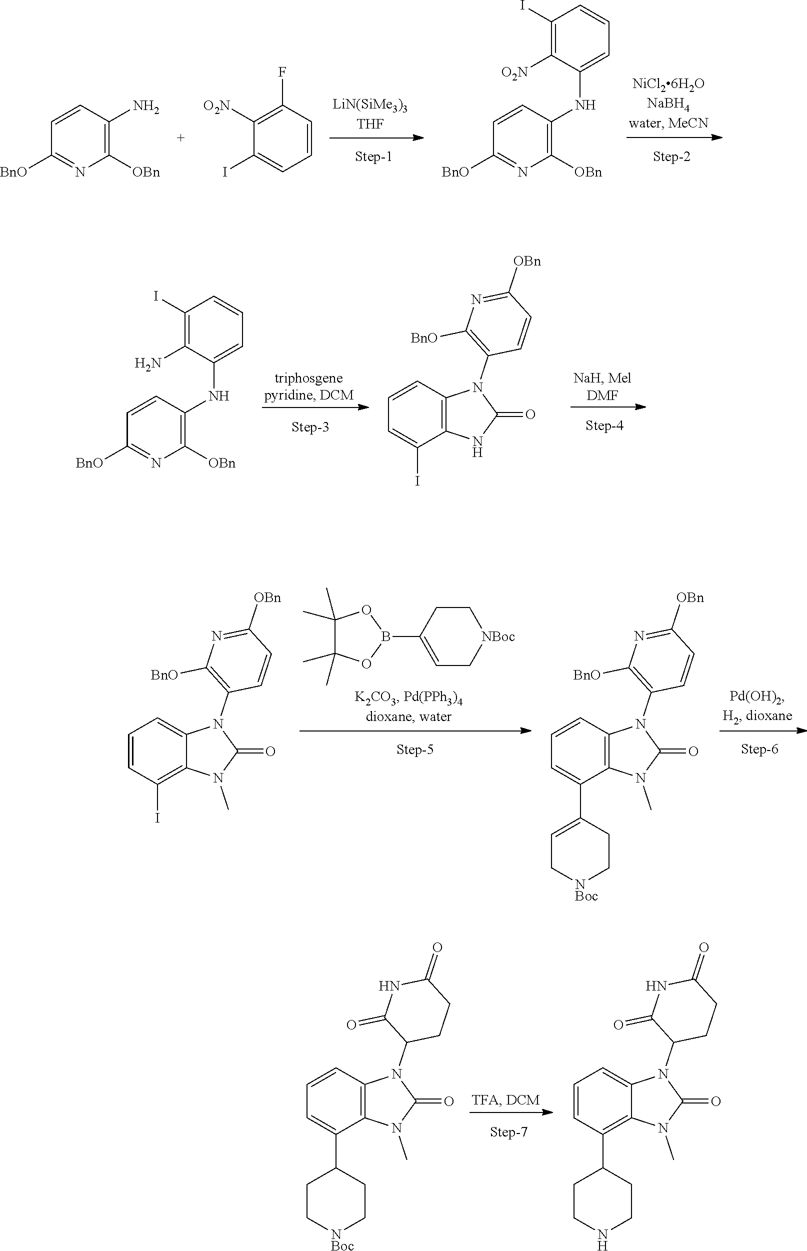

### Step-1:

2,6-dibenzyloxypyridin-3-amine (50 g, 163.21 mmol) was dissolved with THE (500 mL) and cooled to −78° C. Lithium bis(trimethylsilyl)amide (40.96 g, 244.81 mmol) was added dropwise, then stirred for 1 hour at −78° C. 1-fluoro-3-iodo-2-nitro-benzene (43.58 g, 163.21 mmol) was added dropwise as a solution in THE (500 mL) at −78° C., then stirred for 1 hour at −78° C. After the reaction was complete as confirmed by TLC, the reaction was then quenched with 10% ammonium chloride solution (150 mL). The solvent was evaporated to a black gummy solid. Pet ether was added and stirred well for 15 minutes until formation of a brown solid, which was filtered through a Buchner funnel and washed with pet ether (2×300 mL). The filter cake was dried under vacuum to afford 2,6-dibenzyloxy-N-(3-iodo-2-nitro-phenyl)pyridin-3-amine (80 g, 144.57 mmol). LC-MS (ES⁺): m/z 554.20 [M+H]⁺.

### Step-2:

A solution of 2,6-dibenzyloxy-N-(3-iodo-2-nitro-phenyl)pyridin-3-amine (80 g, 144.57 mmol) in acetonitrile (720 mL) and water (80 mL) was added nickel(II) chloride hexahydrate, 98% (8.22 g, 28.91 mmol). The reaction was cooled to 0° C. and sodium borohydride (13.67 g, 361.44 mmol) was added portionwise over 1 hour. The reaction mixture was stirred for 30 minutes at room temperature. Upon completion of the reaction as confirmed by TLC, the reaction was filtered through celite, and washed with ethyl acetate. The organic layers were separated and washed with brine solution and dried over anhydrous Na₂SO₄. The organic layer was evaporated to obtain a black gummy solid. To this crude residue, pet ether was added and stirred until a brown solid was obtained. The solid was filtered through a Buchner funnel, then washed with pet ether and dried under vacuum to afford N1-(2,6-dibenzyloxy-3-pyridyl)-3-iodo-benzene-1,2-diamine (36 g, 66 mmol, 45% yield). LC-MS (ES⁺): m/z 524.23 [M+H]⁺.

### Step-3:

A solution of N1-(2,6-dibenzyloxy-3-pyridyl)-3-iodo-benzene-1,2-diamine (5.58 g, 10.66 mmol) in DCM (120 mL) was cooled to 0° C. Pyridine (8.43 g, 106.62 mmol, 8.62 mL) was added dropwise and the solution stirred for 30 minutes at 0° C. Triphosgene (4.75 g, 15.99 mmol) was added dropwise at 0° C. as a solution. The reaction mixture was stirred for 1 hour at room temperature while monitoring by TLC. Upon completion, the reaction was quenched with saturated NaHCO₃ solution, which was added slowly at 0° C. with observed effervescence. The reaction mass was extracted with DCM, then washed with brine solution and dried over anhydrous Na₂SO₄. The organic layers were evaporated to obtain a pale brown solid. To this crude solid, diethyl ether was added and stirred well, before filtering through a Buchner funnel. The product was washed with diethyl ether and dried under vacuum to afford 3-(2,6-dibenzyloxy-3-pyridyl)-7-iodo-1H-benzimidazol-2-one (5.1 g, 8.9 mmol, 83% yield) LC-MS (ES⁺): m/z 550.55 [M+H]⁺.

### Step-4:

A solution of 3-(2,6-dibenzyloxy-3-pyridyl)-7-iodo-1H-benzimidazol-2-one (47.82 g, 87.06 mmol) in DMF (410 mL) and cooled to 0° C. Sodium hydride (60% dispersion in mineral oil) (5.60 g, 243.75 mmol) was added portion-wise, then the reaction mixture stirred for 30 minutes at room temperature. Iodomethane (18.53 g, 130.58 mmol, 8.13 mL) was added dropwise at 0° C. and the reaction mixture stirred for 1 hour at room temperature. Upon completion of the reaction as confirmed by TLC, the reaction was decanted slowly into ice cold water. An off-white solid precipitated and was filtered through a Buchner funnel, then washed with ice cold water and dried under vacuum. The solid was azeotroped with toluene (2×200 mL) to obtain a pale brown solid. Pet ether was added and stirred well for 10 minutes before filtering the solid through Buchner funnel and washing with pet ether (3×100 mL). The product was dried under vacuum to afford 1-(2,6-dibenzyloxy-3-pyridyl)-4-iodo-3-methyl-benzimidazol-2-one as a light brown solid (47 g, 83 mmol, 95% yield). LC-MS (ES⁺): m/z 564.03 [M+H]⁺.

### Step-5:

To a stirred solution of 1-(2,6-dibenzyloxy-3-pyridyl)-4-iodo-3-methyl-benzimidazol-2-one (25 g, 44.37 mmol) in dioxane (210 mL) and water (90 mL) were added potassium carbonate (18.40 g, 133.12 mmol) and tert-butyl 4-(4,4,5,5-tetramethyl-1,3,2-dioxaborolan-2-yl)-3,6-dihydro-2H-pyridine-1-carboxylate (20.58 g, 66.56 mmol). The reaction mixture was degassed with nitrogen for 10 minutes before palladium triphenylphosphane (5.13 g, 4.44 mmol) was added. The reaction was stirred at 100° C. for 4 hours and monitored by TLC and LC-MS. The reaction mass was filtered and concentrated under vacuum, then purified by column chromatography (100-200 mesh silica gel, 10-20% EtOAc in pet ether) to afford tert-butyl 4-[1-(2,6-dibenzyloxy-3-pyridyl)-3-methyl-2-oxo-benzimidazol-4-yl]-3,6-dihydro-2H-pyridine-1-carboxylate (20 g, 31.63 mmol, 71.27% yield) as a yellow solid. LC-MS (ES⁺): m/z 619.19 [M+H]⁺.

### Step-6:

To a stirred solution of tert-butyl 4-[1-(2,6-dibenzyloxy-3-pyridyl)-3-methyl-2-oxo-benzimidazol-4-yl]-3,6-dihydro-2H-pyridine-1-carboxylate (21.00 g, 33.94 mmol) in 1,4-dioxane (600 mL) was added dihydroxypalladium (5.72 g, 40.73 mmol). The reaction mixture was stirred for 12 hours at 60-65° C. under hydrogen gas at 150 psi. TLC and LC-MS were checked to confirm reaction completion (10% methanol in DCM, R_{f} value: 0.4). Upon completion, the reaction was filtered through celite and washed with ethyl acetate. The filtrate was concentrated under reduced pressure to give the crude product, which was triturated with diethyl ether to afford tert-butyl 4-[1-(2,6-dibenzyloxy-3-pyridyl)-3-methyl-2-oxo-benzimidazol-4-yl]-3,6-dihydro-2H-pyridine-1-carboxylate (10 g, 19.98 mmol, 58.86% yield). LC-MS (ES⁺): m/z 441.54 [M+H]⁺.

### Step-7:

A solution of tert-butyl 4-[1-(2,6-dioxo-3-piperidyl)-3-methyl-2-oxo-benzimidazol-4-yl]piperidine-1-carboxylate (10 g, 22.60 mmol) in DCM (150 mL) was cooled to 0° C. Trifluoroacetic acid (25.77 g, 225.99 mmol, 17.41 mL) was added and the reaction mixture stirred for 12 hours at room temperature. TLC confirmed reaction completion (10% methanol in DCM, R_{f} value: 0.2). Upon completion the reaction solvent was evaporated, and diethyl ether (2×100 mL) added to the crude mixture. Diethyl ether was removed and the product was dried under vacuum to afford 3-[3-methyl-2-oxo-4-(4-piperidyl)benzimidazol-1-yl]piperidine-2,6-dione (10.71 g, 22.39 mmol, 99.06% yield, 95.40% purity, TFA salt) as an off-white solid. LC-MS (ES⁺): m/z 343.33 [M+H]⁺.

### Synthesis of 3-[3-methyl-4-[4-(methylamino)-1-piperidyl]-2-oxo-benzimidazol-1-yl]piperidine-2,6-dione

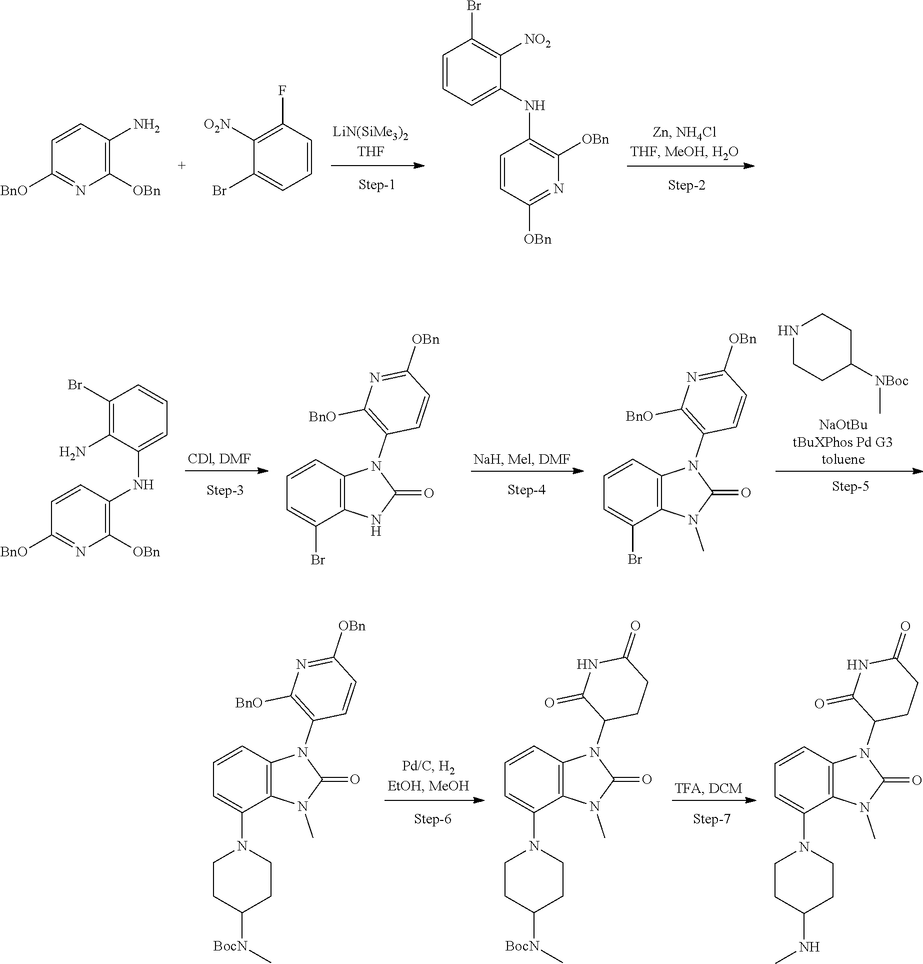

### Step-1:

A solution of 2,6-dibenzyloxypyridin-3-amine (2 g, 6.53 mmol) in THF (50 mL) was cooled to −78° C. To this was added lithium bis(trimethylsilyl)azanide (1.09 g, 6.53 mmol, 6.5 mL) dropwise over 15 minutes at −78° C. The reaction was maintained at −78° C. for 1 hour, followed by the dropwise addition of 1-bromo-3-fluoro-2-nitro-benzene (1.44 g, 6.53 mmol). The reaction mixture was stirred for another 2 hours. Completion of the reaction was confirmed by TLC (20% EtOAc/Pet ether) and LC-MS. The reaction mixture was diluted with 10% ammonium chloride solution and concentrated under reduced pressure. The crude material was purified by column chromatography (pet ether and ethyl acetate) to afford 2,6-dibenzyloxy-N-(3-bromo-2-nitro-phenyl)pyridin-3-amine (2.5 g, 4.08 mmol, 62.42% yield) as a yellow solid. LC-MS (ES⁺): m/z 506.32 [M+H]⁺.

### Step-2:

To a stirred solution of 2,6-dibenzyloxy-N-(3-bromo-2-nitro-phenyl)pyridin-3-amine (20 g, 39.50 mmol) in THE (65 mL) and methanol (65 mL) was added zinc (25.83 g, 394.99 mmol, 3.62 mL) followed by the addition of a suspension of ammonia hydrochloride (31.69 g, 592.48 mmol) in water (65 mL). The reaction mixture was stirred at room temperature for 2 h and the progress of the reaction monitored by TLC. Upon completion of the reaction, the contents were passed through a celite bed. The filtrate was concentrated under vacuum and extracted by EtOAc (250 ml). The organic layers were separated and dried over anhydrous Na₂SO₄, then evaporated under vacuum. The crude material was purified by column chromatography using Devisil silica (eluting solvent 0-70% EtOAc in hexane) to afford 3-bromo-N1-(2,6-dibenzyloxy-3-pyridyl)benzene-1,2-diamine (15 g, 27.56 mmol, 69.78% yield) as a brown solid. LC-MS (ES⁺): m/z 398.46 [M-Br+H]⁺.

### Step-3:

A solution of 3-bromo-N1-(2,6-dibenzyloxy-3-pyridyl)benzene-1,2-diamine (210 g, 440.84 mmol) in DMF (1.17 L) was added di(imidazol-1-yl)methanone (200.15 g, 1.23 mol) at room temperature. The reaction mixture was stirred for 16 hours at room temperature. TLC confirmed the consumption of starting material (40% ethyl acetate in pet ether, R_{f} value: 0.4). Upon completion the reaction, the mixture was poured into ice cold water. An off-white solid was precipitated and filtered through Buchner funnel. The wet solid was washed with water and dried under vacuum to afford 7-bromo-3-(2,6-dibenzyloxy-3-pyridyl)-1H-benzimidazol-2-one (220 g, 378.33 mmol, 85.82% yield). LC-MS (ES⁻): m/z 500.41 [M−H]⁻.

### Step-4:

To a stirred solution of 7-bromo-3-(2,6-dibenzyloxy-3-pyridyl)-1H-benzimidazol-2-one (220 g, 437.93 mmol) in DMF (2200 mL) was added sodium hydride, 60% dispersion in mineral oil, (28.19 g, 1.23 mol) at 0° C. The reaction mixture was warmed to RT and maintained for 1 h. The reaction was cooled again to 0° C. and iodomethane (93.24 g, 656.90 mmol, 40.89 mL) added dropwise at 0-5° C. The reaction mass was allowed to warm to RT and maintained for 1 h. The progress of the reaction was followed and confirmed by TLC (20% ethyl acetate:pet ether

Rf value: 0.3). Upon completion, the reaction was quenched into ice cold water and an off-white solid precipitated that was isolated by vacuum filtration & Buchner funnel, and washed with water (1000 mL). The wet solid obtained was dried under vacuum to afford 4-bromo-1-(2,6-dibenzyloxy-3-pyridyl)-3-methyl-benzimidazol-2-one as an off-white solid (221 g, 420.66 mmol, 96.05% yield). LC-MS (ES⁺): m/z 516.09 [M+H]⁺.

### Step-5:

In a sealed tube, a solution of 4-bromo-1-(2,6-dibenzyloxy-3-pyridyl)-3-methyl-benzimidazol-2-one (0.5 g, 968.27 μmol), tert-butyl N-methyl-N-(4-piperidyl)carbamate (207.50 mg, 968.27 μmol) in toluene (10 mL) was added sodium 2-methylpropan-2-olate (279.16 mg, 2.90 mmol). The reaction was degassed with argon for 15 minutes, then tBuXPhos PdG3 (76.88 mg, 96.83 μmol) was added to the reaction mixture and degassed for another 5 minutes. The reaction mixture was then heated at 90° C. for 5 hours. The progress of the reaction was monitored by LC-MS. The reaction mixture was filtered through a celite bed and the filtrate concentrated to give the crude compound which was purified by column chromatography (100-200 mesh silica gel, 0-70% ethyl acetate in pet ether) to afford tert-butyl N-[1-[1-(2,6-dibenzyloxy-3-pyridyl)-3-methyl-2-oxo-benzimidazol-4-yl]-4-piperidyl]-N-methyl-carbamate (0.25 g, 307.80 μmol, 31.79% yield) as a yellow liquid. LC-MS (ES⁺): m/z 672.41 [M+Na]⁺.

### Step-6:

Tert-butyl N-[1-[1-(2,6-dibenzyloxy-3-pyridyl)-3-methyl-2-oxo-benzimidazol-4-yl]-4-piperidyl]-N-methyl-carbamate (0.415 g, 638.68 μmol) was solvated in ethanol (3 mL) and methanol (3 mL), and purged with nitrogen for 10 minutes. To this solution was added palladium, 10% on carbon, Type 487, dry (67.97 mg, 638.68 μmol) and the reaction mixture stirred under a hydrogen atmosphere (rubber bladder) at RT for 5 hr. The progress of reaction was monitored by TLC (10% methanol DCM; Rf value: 0.3). After completion the reaction mixture was filtered through a celite bed and washed with methanol (50 mL×2) and the organic layer concentrated to furnish the product tert-butyl N-[1-[1-(2,6-dioxo-3-piperidyl)-3-methyl-2-oxo-benzimidazol-4-yl]-4-piperidyl]-N-methyl-carbamate (0.3 g, 610.75 μmol, 95.63% yield) as a brown solid. LC-MS (ES⁺): m/z 672.41 [M+H]⁺.

### Step-7:

To a stirred solution of tert-butyl N-[1-[1-(2,6-dioxo-3-piperidyl)-3-methyl-2-oxo-benzimidazol-4-yl]-4-piperidyl]-N-methyl-carbamate (0.3 g, 636.20 μmol) in DCM (50 mL) at 0° C. was added TFA (72.54 mg, 636.20 μmol, 49.01 μL) over 10 minutes. The reaction mixture was stirred at 25° C. for 4 hours and the reaction was monitored by TLC (10% methanol in DCM, R_{f} value: 0.2). After reaction completion, the mixture was concentrated and co-distilled with toluene (10 ml) and diethyl ether (2×50 ml) to afford the product 3-[3-methyl-4-[4-(methylamino)-1-piperidyl]-2-oxo-benzimidazol-1-yl]piperidine-2,6-dione TFA salt (0.23 g, 447.87 μmol, 70.40% yield) as a brown solid. LC-MS (ES⁺): m/z 372.28 [M+H]⁺.

### Synthesis of 3-[3-methyl-5-(4-piperidyl) indol-1-yl]piperidine-2,6-dione

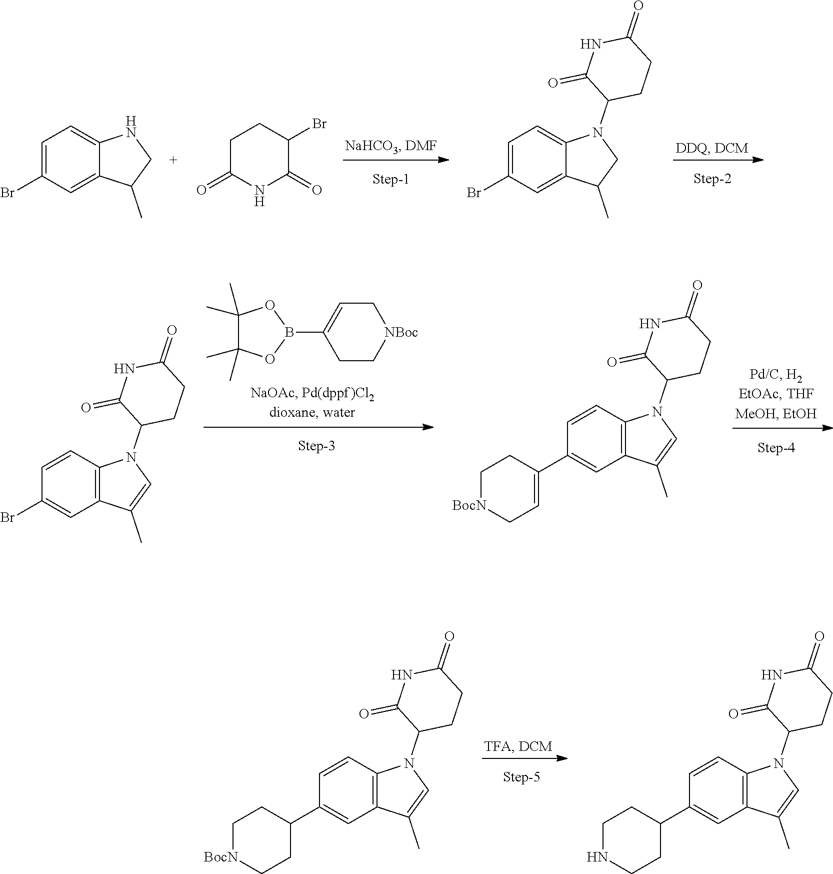

### Step-1:

To a solution of 5-bromo-3-methyl-indoline (5.5 g, 25.93 mmol) in DMF (70 mL) in a sealed tube, was added 3-bromopiperidine-2,6-dione (7.47 g, 38.90 mmol) and sodium bicarbonate (6.54 g, 77.80 mmol, 3.03 mL). The reaction mixture was stirred under heating at 70° C. for 48 h. The reaction was monitored by TLC and LC-MS, then poured into ice water and extracted with ethyl acetate. The organic layers were washed with brine water, dried over Na₂SO₄, and concentrated under reduced pressure. The crude was purified by column chromatography using 30% ethyl acetate in hexane as eluent to afford 3-(5-bromo-3-methyl-indolin-1-yl)piperidine-2,6-dione (2.5 g, 7.43 mmol, 28.64% yield, 96% purity). LC-MS (ES⁺): m/z 323.26 [M+H]⁺.

### Step-2:

To a stirred solution of compound 3-(5-bromo-3-methyl-indolin-1-yl)piperidine-2,6-dione (2.5 g, 7.74 mmol) in DCM (80 mL) was added DDQ (2.11 g, 9.28 mmol) slowly at 0° C. After addition, stirring was continued for 1 hour at room temperature. The reaction was monitored by LC-MS and TLC. After product formation was confirmed by LC-MS, the reaction mixture was extracted with DCM and the organic layers washed with 1M NaOH. Then the organic layer was dried over Na₂SO₄ and evaporated under reduced pressure. The crude was purified by column chromatography (silica gel, 40% ethyl acetate in hexane) to afford 3-(5-bromo-3-methyl-indol-1-yl)piperidine-2,6-dione (911.38 mg, 2.83 mmol, 36.58% yield). LC-MS (ES⁺): m/z 321.11 [M+H]⁺.

### Step-3:

3-(5-bromo-3-methyl-indol-1-yl)piperidine-2,6-dione (0.05 g, 155.68 μmol) was charged into a 250 mL round-bottomed flask and solvated in 1,4-dioxane (2 mL) and water (0.2 mL). Tert-butyl 4-(4,4,5,5-tetramethyl-1,3,2-dioxaborolan-2-yl)-3,6-dihydro-2H-pyridine-1-carboxylate (62.58 mg, 202.39 μmol) and sodium acetate, anhydrous (38.31 mg, 467.04 μmol) were added at room temperature under argon gas. The reaction mixture was degassed with argon for 20 minutes. After degassing, cyclopentyl(diphenyl)phosphane; dichloropalladium; iron (11.39 mg, 15.57 μmol) was added and the reaction was heated at 80° C. for 6 hours, while monitoring with TLC and LC-MS. The catalyst was filtered through celite and washed with ethyl acetate (10 mL×3). The filtrate was concentrated under reduced pressure to obtain the crude product which was purified by column chromatography (silica gel 100-200 mesh, 0-50% ethyl acetate in pet ether) to afford tert-butyl 4-[1-(2,6-dioxo-3-piperidyl)-3-methyl-indol-5-yl]-3,6-dihydro-2H-pyridine-1-carboxylate (0.04 g, 49.11 μmol, 31.55% yield) as a grey colored solid. LC-MS (ES⁻): m/z 422.51 [M−H]⁻.

### Step-4:

Tert-butyl 4-[1-(2,6-dioxo-3-piperidyl)-3-methyl-indol-5-yl]-3,6-dihydro-2H-pyridine-1-carboxylate (0.1 g, 236.13 μmol) was charged into a round-bottom flask and solvated in ethyl acetate (2 mL). To this stirring solution was added palladium, 10% on carbon, type 487, dry (25.13 mg, 236.13 μmol), then H₂ pressure was applied from a bladder and the reaction was stirred continuously at room temperature for 16 hours. The reaction progress was checked by LC-MS, then the reaction mixture was filtered through a celite bed, washed with ethyl acetate (10 mL) and methanol (10 mL)/The filtrate was concentrated under reduced pressure to get the crude product, which was triturated with n-pentane (5 mL) and concentrated under reduced pressure to obtain the desired product tert-butyl 4-[1-(2,6-dioxo-3-piperidyl)-3-methyl-indol-5-yl]piperidine-1-carboxylate (0.08 g, 131.60 μmol, 55.73% yield) as a grey color solid. LC-MS (ES⁻): m/z 424.34 [M−H]⁻.

### Step-5:

To a stirred solution of tert-butyl 4-[1-(2,6-dioxo-3-piperidyl)-3-methyl-indol-5-yl]piperidine-1-carboxylate (0.03 g, 70.50 μmol) in DCM (5 mL) was added TFA (40.19 mg, 352.51 μmol, 27.16 μL) at 0° C. The reaction was stirred for 16 hours at room temperature. The reaction progress was monitored by LC-MS. After the completion of reaction, the solvent was evaporated under vacuum to obtain crude product which was triturated in diethyl ether (10 mL) and then filtered to afford 3-[3-methyl-5-(4-piperidyl) indol-1-yl]piperidine-2,6-dione TFA salt (0.015 g, 29.36 μmol, 41.64% yield) as a grey color solid. LC-MS (ES⁺): m/z 326.35 [M+H]⁺.

### Synthesis of 3-[3-methyl-5-[4-(methylamino)-1-piperidyl]-2-oxo-benzimidazol-1-yl]piperidine-2,6-dione

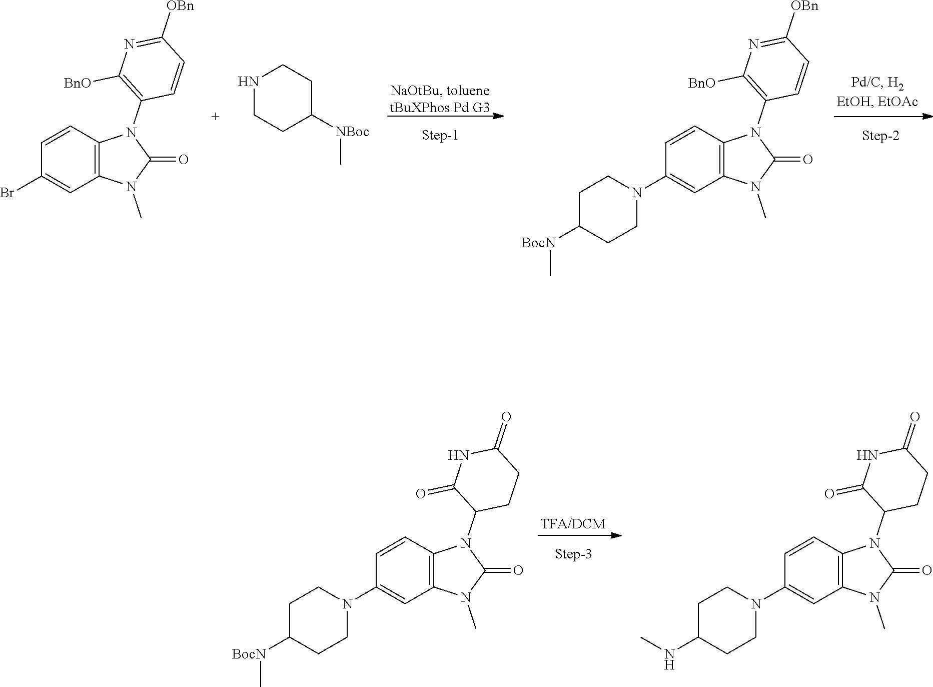 [SMILES: Cn1c(=O)n(-c2ccc(OCc3ccccc3)nc2OCc2ccccc2)c2ccc(Br)cc21 ; CN(C(=O)OC(C)(C)C)C1CCNCC1 ; "NaOtBu, toluenetBuXPhos Pd G3" ; CN(C(=O)OC(C)(C)C)C1CCN(c2ccc3c(c2)n(C)c(=O)n3-c2ccc(OCc3ccccc3)nc2OCc2ccccc2)CC1 ; CN(C(=O)OC(C)(C)C)C1CCN(c2ccc3c(c2)n(C)c(=O)n3C2CCC(=O)NC2=O)CC1 ; CNC1CCN(c2ccc3c(c2)n(C)c(=O)n3C2CCC(=O)NC2=O)CC1]

### Step-1:

In a sealed tube, a solution of 5-bromo-1-(2,6-dibenzyloxy-3-pyridyl)-3-methyl-benzimidazol-2-one (1 g, 1.94 mmol) and tert-butyl N-methyl-N-(4-piperidyl)carbamate (622.52 mg, 2.90 mmol) in toluene (60 mL) was added sodium 2-methylpropan-2-olate (558.30 mg, 5.81 mmol). The reaction was degassed with argon for 15 minutes, then tBuXPhos PdG3 (153.76 mg, 193.65 μmol) was added to the reaction mixture and degassed again for 5 minutes. The reaction mixture was then heated at 90° C. for 16 hours, and the progress of the reaction monitored by LC-MS. The reaction mixture was filtered through celite bed and the filtrate was concentrated in vacuo and then purified by column chromatography (100-200 mesh silica gel, 0-70% ethyl acetate in pet ether) to afford tert-butyl N-[1-[1-(2,6-dibenzyloxy-3-pyridyl)-3-methyl-2-oxo-benzimidazol-5-yl]-4-piperidyl]-N-methyl-carbamate (0.7 g, 1.02 mmol, 52.85% yield) as a yellow liquid. LC-MS (ES⁺): m/z 650.97 [M+H]⁺.

### Step-2:

A stirring solution of tert-butyl N-[1-[1-(2,6-dibenzyloxy-3-pyridyl)-3-methyl-2-oxo-benzimidazol-5-yl]-4-piperidyl]-N-methyl-carbamate (0.6 g, 923.39 μmol) in a mixture of ethanol (50 mL) and ethyl acetate (50 mL) was purged with hydrogen gas followed by addition of palladium, 10% on carbon, type 487, dry (523.21 mg, 4.92 mmol) and concentrated HCl (254.14 mg, 7.06 mmol, 2 mL). The reaction mixture was stirred under hydrogen atmosphere (1 atm pressure) at room temperature for 5 hours. The progress of the reaction monitored by LC-MS. After complete consumption of the starting material, the reaction mixture was filtered through a celite bed and washed with methanol (50 mL×2). The filtrate was concentrated to furnish the tert-butyl N-[1-[1-(2,6-dioxo-3-piperidyl)-3-methyl-2-oxo-benzimidazol-5-yl]-4-piperidyl]-N-methyl-carbamate (0.25 g, 334.01 μmol, 36.17% yield) as a yellow solid. LC-MS (ES⁺): m/z 472.93 [M+H]⁺.

### Step-3:

To a stirred solution of tert-butyl N-[1-[1-(2,6-dioxo-3-piperidyl)-3-methyl-2-oxo-benzimidazol-5-yl]-4-piperidyl]-N-methyl-carbamate (0.32 g, 678.62 μmol) at 0° C. was added TFA (77.38 mg, 678.62 μmol, 52.28 μL) over 10 minutes. The reaction mixture was stirred at 25° C. for 4 hours and the reaction monitored by TLC (10% methanol in DCM R_{f} value: 0.2). After completion, the reaction mixture was concentrated and co-distilled with toluene (10 ml) and diethyl ether (2×50 ml) to afford the product 3-[3-methyl-5-[4-(methylamino)-1-piperidyl]-2-oxo-benzimidazol-1-yl]piperidine-2,6-dione TFA salt (0.25 g, 475.69 μmol, 70.10% yield) as an off-white solid. LC-MS (ES⁺): m/z 372.28 [M+H]⁺.

### Synthesis of 3-[3-methyl-5-[[4-(methylamino)-1-piperidyl]methyl]-2-oxo-benzimidazol-1-yl]piperidine-2,6-dione

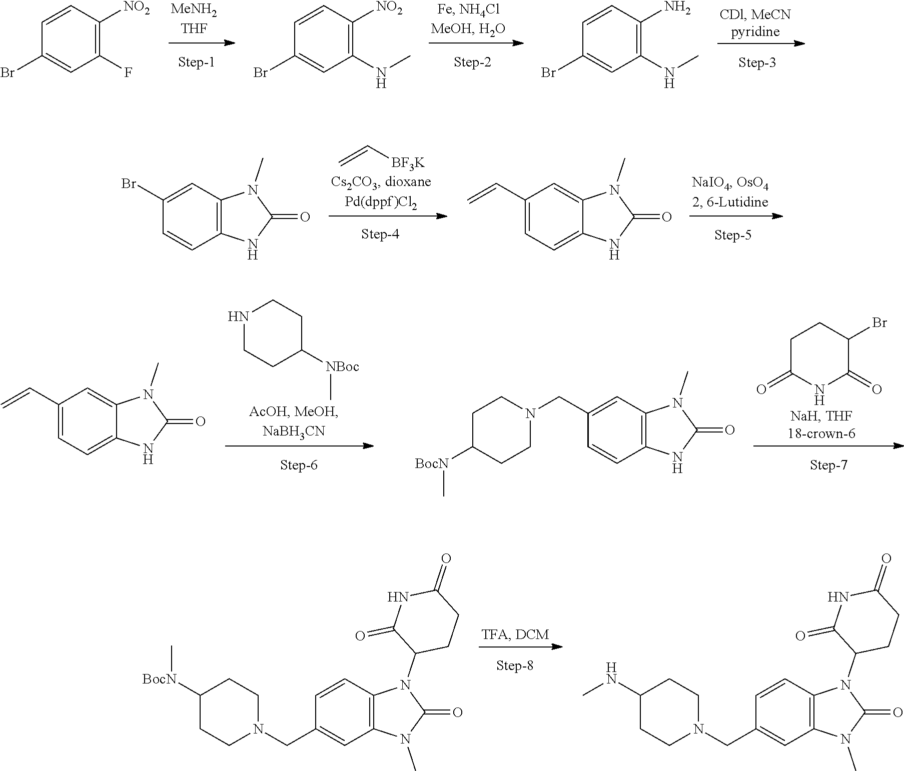

### Step-1:

To a stirred solution of 4-bromo-2-fluoro-1-nitro-benzene (10 g, 4.55 mmol) in THE (100 mL) cooled to 0° C., methanamine (141.17 g, 4.55 mmol, 157.03 μL) was added dropwise. The reaction was heated to 60° C. for 16 hours and the progress of the reaction was monitored by LC-MS and TLC (10% EtOAc in pet ether; R_{f}=0.7). The reaction was concentrated to give the crude solid which was washed with pentane twice and dried to afford 5-bromo-N-methyl-2-nitro-aniline (600 mg, 2.49 mmol, 54.85% yield). LC-MS (ES⁺): m/z 231.24 [M+H]⁺.

### Step-2:

To a stirred solution of 5-bromo-N-methyl-2-nitro-aniline (5 g, 10.82 mmol) in ethanol (50 mL) and water (50 mL) was added iron (3.02 g, 54.10 mmol) and ammonium chloride, 98+% (2.89 g, 54.10 mmol). The reaction was heated to about 90° C. for 16 hours and the progress of the reaction was monitored by LC-MS and TLC (30% EtOAc in pet ether, R_{f}=0.5). The reaction was filtered through celite, concentrated, diluted with water, and extracted with EtOAc. The organic layer was washed with brine solution, dried over sodium sulfate, and the solvent evaporated. The crude was washed with diethyl ether and pentane to afford 4-bromo-N2-methyl-benzene-1,2-diamine (4 g, 954.92 mmol, 88.24% yield) as a dark red liquid. LC-MS (ES⁺): m/z 203.27 [M+2H]⁺.

### Step-3:

To the stirred solution of 4-bromo-N2-methyl-benzene-1,2-diamine (5 g, 24.87 mmol) in acetonitrile (40 mL) was added di(imidazol-1-yl)methanone (24.19 g, 149.21 mmol) and pyridine (5.90 g, 74.60 mmol, 6.03 mL). The reaction mixture was heated to 85° C. for 16 hours and the progress of the reaction was monitored by LC-MS and TLC (50% EtOAc in pet ether). The reaction mixture was poured into cold water, the precipitated crude product was filtered, and then washed with excess of cold water to remove pyridine. The obtained crude compound was washed with diethyl ether and pentane to give 5-bromo-3-methyl-1H-benzimidazol-2-one (5 g, 21.58 mmol, 86.78% yield) as an off-white solid. LC-MS (ES⁺): m/z 227.17 [M+H]⁺.

### Step-4:

To a stirred solution of 5-bromo-3-methyl-1H-benzimidazol-2-one (2.5 g, 11.01 mmol) in dioxane (25 mL) was added potassium trifluoro(vinyl)boranuide (1.47 g, 11.01 mmol) and cesium carbonate (3.59 g, 11.01 mmol). The reaction was purged with argon for 15 minutes followed by the addition of cyclopentyl(diphenyl)phosphane; dichloromethane; dichloropalladium; iron (899.15 mg, 1.10 mmol). The reaction was heated to 90° C. for 4 hours and the progress of the reaction was monitored by LC-MS and TLC (50% ethyl acetate in pet ether, R_{f}=0.6). The reaction mixture was quenched with cold water and extracted with ethyl acetate. The organic layer was washed with brine solution, dried over sodium sulfate, and the solvent concentrated to get crude which was purified by reverse phase column chromatography (1% ammonium acetate/acetonitrile) to afford 3-methyl-5-vinyl-1H-benzimidazol-2-one (1.5 g, 7.75 mmol, 70.39% yield) as an off-white solid. LC-MS (ES⁺): m/z 175.38 [M+H]⁺.

### Step-5:

A stirred solution of 3-methyl-5-vinyl-1H-benzimidazol-2-one (1.5 g, 8.61 mmol) in 1,4-dioxane (15 mL) and water (15 mL) was cooled to 0° C. and 2,6-dimethylpyridine (1.85 g, 17.22 mmol, 2.00 mL) was added. This was followed by the addition of sodium periodate (3.68 g, 17.22 mmol) and tetraoxoosmium (218.91 mg, 861.09 μmol). The reaction mass was stirred at 28° C. for 2 hours and the progress of the reaction was monitored by LC-MS and TLC (50% Et OAc in Pet ether, R_{f}=0.5). The reaction mixture was quenched with EtOAc, filtered, and concentrated to give the crude product, which was purified by reverse phase column chromatography (1% ammonium acetate/acetonitrile) to afford 3-methyl-2-oxo-1H-benzimidazole-5-carbaldehyde (1 g, 5.62 mmol, 65.26% yield) as a brown color solid. LC-MS (ES⁺): m/z 177.39 [M+H]⁺.

### Step-6:

To a stirred solution of 3-methyl-2-oxo-1H-benzimidazole-5-carbaldehyde (2.0 g, 11.35 mmol) in methanol (20 mL) cooled to 0° C. was added acetic acid (681.72 mg, 11.35 mmol, 649.26 μL) and tert-butyl N-methyl-N-(4-piperidyl)carbamate (2.43 g, 11.35 mmol), and molecular sieves. The reaction was heated to 65° C. for 4 hours, then cooled to 0° C. Sodium cyanoborohydride (713.39 mg, 11.35 mmol) was added portion-wise over a period of 15 minutes. The reaction was stirred at 28° C. for 16 hours. The progress of the reaction was monitored by LC-MS and TLC (50% ethyl acetate in pet ether, R_{f}=0.5). The reaction mixture was quenched with water (5 ml) and concentrated to get the crud product, which was purified by reverse phase column chromatography (1% ammonium acetate/acetonitrile) to get partially purified compound tert-butyl N-methyl-N-[1-[(3-methyl-2-oxo-1H-benzimidazol-5-yl)methyl]-4-piperidyl]carbamate (2.0 g, 3.63 mmol, 31.99% yield). This was taken for next step without further purification. LC-MS (ES⁺): m/z 375.35 [M+H]⁺.

### Step-7:

A stirred solution of tert-butyl N-methyl-N-[1-[(3-methyl-2-oxo-1H-benzimidazol-5-yl)methyl]-4-piperidyl]carbamate (2.5 g, 6.68 mmol) in THE (DRY) (25 mL) was cooled to 0° C. and NaH (767.41 mg, 33.38 mmol) was added in portions, followed by 18-crown-6 (882.28 mg, 3.34 mmol, 747.70 μL). The reaction mass stirred at 28° C. for 2 hours, cooled to 0° C., and 3-bromopiperidine-2,6-dione (1.28 g, 6.68 mmol) was added. The reaction was stirred at 65° C. for 6 hours while the progress of the reaction was monitored by LC-MS and TLC (50% EtOAc in pet ether, R_{f}=0.5). The reaction mixture was quenched with cold water and extracted with ethyl acetate. The organic layer was separated, washed with brine solution, and dried over sodium sulfate. The solvent was evaporated to get crude compound which was purified using prep-HPLC to afford tert-butyl N-[1-[[1-(2,6-dioxo-3-piperidyl)-3-methyl-2-oxo-benzimidazol-4-yl]methyl]-4-piperidyl]-N-methyl-carbamate (600 mg, 1.09 mmol, 16.29% yield) as an off-white solid. LC-MS (ES⁺): m/z 486.95 [M+H]⁺.

### Step-8:

A stirred solution of tert-butyl N-[1-[[1-(2,6-dioxo-3-piperidyl)-3-methyl-2-oxo-benzimidazol-5-yl]methyl]-4-piperidyl]-N-methyl-carbamate (50 mg, 102.97 μmol) in DCM (2 mL) was cooled to 0° C. and TFA (117.41 mg, 1.03 mmol, 79.33 μL) was added. The reaction mixture was stirred at 28° C. for 2 hours; the progress of the reaction was monitored by LC-MS and TLC (10% MeOH in DCM, R_{f}=0.4). The reaction mixture was concentrated to remove DCM and excess TFA. The crude product was isolated and cooled to 0° C. and washed with cold diethyl ether (5 ml×3) and pentane, then lyophilized to give 3-[3-methyl-5-[[4-(methylamino)-1-piperidyl]methyl]-2-oxo-benzimidazol-1-yl]piperidine-2,6-dione TFA salt (18 mg, 31.11 μmol, 30.21% yield) as an off-white solid. LC-MS (ES⁺): m/z 386.32 [M+H]⁺.

### Synthesis of 3-[4-[4-(methylamino)-1-piperidyl]anilino]piperidine-2,6-dione

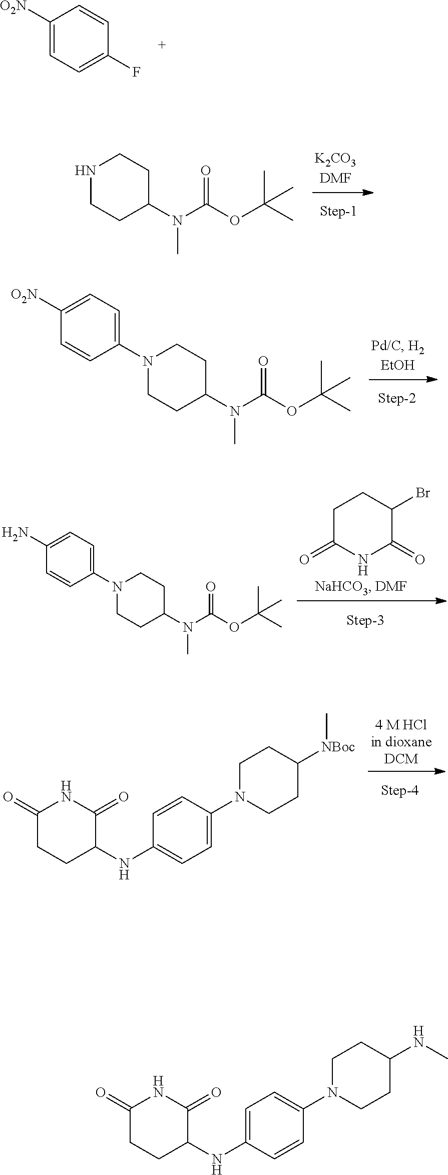

### Step-1:

To a stirred solution of 1-fluoro-4-nitro-benzene (2 g, 14.17 mmol, 1.50 mL) in DMF (10 mL) was added tert-butyl N-methyl-N-(4-piperidyl)carbamate (3.04 g, 14.17 mmol) and potassium carbonate granular (3.92 g, 28.35 mmol) and the reaction was heated to 80° C. for 4 hours. TLC (R_{f}: 0.4 in 10% Ethyl acetate in pet ether) and LC-MS were checked for completion of the reaction. After completion, the reaction was concentrated under vacuum to get the crude product which was purified by flash column chromatography (silica gel) to afford tert-butyl N-methyl-N-[1-(4-nitrophenyl)-4-piperidyl]carbamate (2 g, 5.84 mmol, 41.23% yield). LC-MS (ES⁺): m/z 336.28 [M+H]⁺.

### Step-2:

To the stirred solution of tert-butyl N-methyl-N-[1-(4-nitrophenyl)-4-piperidyl]carbamate (2 g, 5.96 mmol) in ethanol (20 mL) was added palladium, 10% on carbon, type 487, dry (634.59 mg, 5.96 mmol) and the reaction was stirred under H2 atmosphere for 4 hours. TLC (R_{f}. 0.4 in 50% ethyl acetate in pet ether) and LCMS were checked for completion of reaction. The reaction was filtered through a celite bed and washed with methanol. The solvent was evaporated under vacuum, and the residue was washed with pentane to afford tert-butyl N-[1-(4-aminophenyl)-4-piperidyl]-N-methyl-carbamate (1.5 g, 4.67 mmol, 78.24% yield). LC-MS (ES⁺): m/z 303.31 [M+H]⁺.

### Step-3:

Tert-butyl N-[1-(4-aminophenyl)-4-piperidyl]-N-methyl-carbamate (1 g, 3.27 mmol) was dissolved in DMF (10 mL), and 3-bromopiperidine-2,6-dione (1.26 g, 6.55 mmol) and sodium bicarbonate (1.10 g, 13.10 mmol) were added. The reaction was heated to 100° C. for 16 hours. The reaction progress was monitored by TLC (R_{f}: 0.4 in 50% ethyl acetate in pet ether) which showed consumption of starting material. The reaction mixture was then quenched with water, extracted with ethyl acetate, and the organic layers were washed with brine, then dried over anhydrous Na₂SO₄ and concentrated under reduced pressure. The crude compound was purified by column chromatography (100-200 mesh silica gel, ethyl acetate and pet ether) to obtain tert-butyl N-[1-[4-[(2,6-dioxo-3-piperidyl)amino]phenyl]-4-piperidyl]-N-methyl-carbamate (0.6 g, 1.35 mmol, 41.36% yield) as a pale yellow solid. LC-MS (ES⁺): m/z 417.56 [M+H]⁺.

### Step-4:

To a stirred solution of tert-butyl N-[1-[4-[(2,6-dioxo-3-piperidyl)amino]phenyl]-4-piperidyl]-N-methyl-carbamate (0.5 g, 1.20 mmol) in DCM (5 mL), was added 4 M HCl in dioxane (43.77 mg, 1.20 mmol) slowly at 0° C. and the reaction was stirred at 0-25° C. for 2 hours. TLC (R_{f}: 0.4 in 50% ethyl acetate in pet ether) and LCMS were checked for completion of reaction. After completion, the reaction was concentrated under reduced pressure and washed with pentane and diethyl ether to afford 3-[4-[4-(methylamino)-1-piperidyl]anilino]piperidine-2,6-dione HCl salt (0.35 g, 823.28 μmol, 68.58% yield). LC-MS (ES⁺): m/z 317.37 [M+H]⁺.

### Synthesis of 3-[4-[[4-(methylamino)-1-piperidyl]methyl]anilino]piperidine-2,6-dione

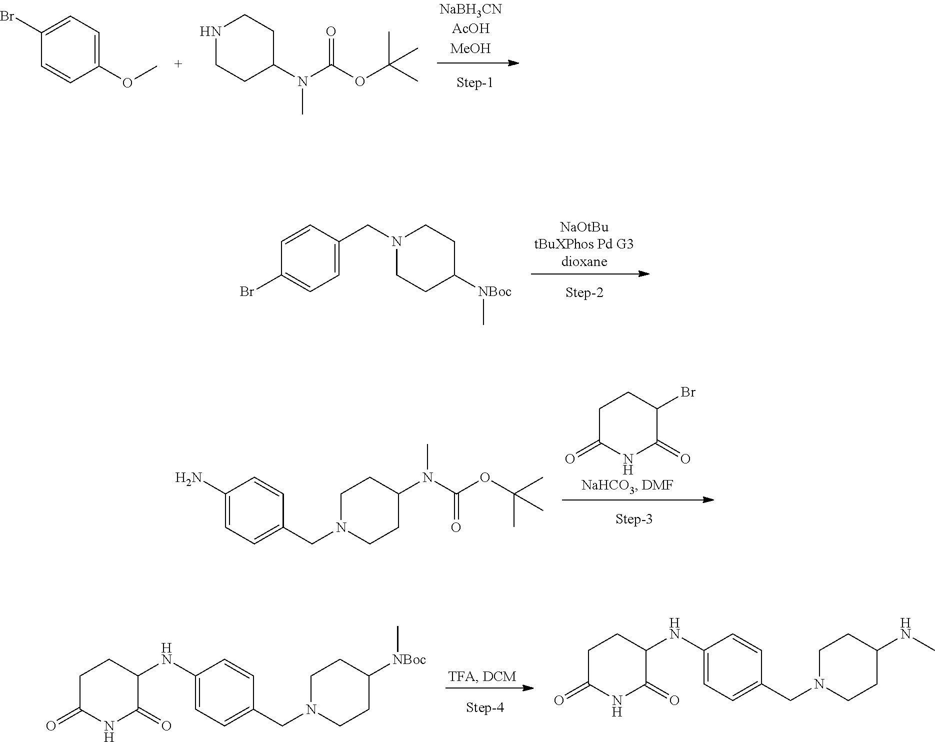

### Step-1:

To a stirred solution of 4-bromobenzaldehyde (1 g, 5.40 mmol), tert-butyl N-methyl-N-(4-piperidyl)carbamate (1.16 g, 5.40 mmol) in methanol (30 mL) was added acetic acid (324.57 mg, 5.40 mmol, 309.12 μL). Then reaction mixture was stirred at room temperature for 10 minutes under N₂ atmosphere. Then sodium cyanoborohydride (679.29 mg, 10.81 mmol) was slowly added. Then the reaction mixture was stirred at room temperature for 16 hours. TLC and LC-MS were checked for completion of reaction. After completion of the reaction, the reaction mixture was concentrated under vacuum. Then the workup was done by using EtOAc and water. The combined organic layer was concentrated under reduced pressure. The crude was further purified using silica gel flash column chromatography to afford tert-butyl N-[1-[(4-bromophenyl)methyl]-4-piperidyl]-N-methyl-carbamate (1.06, 2.77 mmol, 51.16% yield). ¹H NMR (400 MHz, CDCl₃) δ: 7.49 (dd, J=8.4 & 8.2 Hz 2H), 7.23 (dd, J=8.4 & 3.2 Hz 2H), 4.65 (s, 2H), 3.42 (s, 2H), 2.90 (m, 2H), 2.72 (s, 3H), 2.04 (m, 2H), 1.99 (m, 1H), 1.72 (m, 2H), 1.57 (s, 9H).

### Step-2:

To a stirred solution of tert-butyl N-[1-[(4-bromophenyl)methyl]-4-piperidyl]-N-methyl-carbamate (1 g, 2.61 mmol) in 1,4 dioxane (10 mL) was added sodium tert-butoxide (626.78 mg, 6.52 mmol) and ammonia gas by purging for 15 minutes at 0° C. This was followed by the addition of tBuXPhos Pd G3 (310.82 mg, 391.32 μmol) and the reaction was stirred for 16 hours at 90° C. The progress of the reaction was monitored by TLC (40% EtOAc:PE, R_{f} value: 0.3) and LC-MS. After completion of the reaction, the reaction mixture was filtered through celite bed and the filtrate concentrated to a crude that was purified by flash column chromatography (neutral alumina, 40% ethyl acetate in pet ether) to afford tert-butyl N-[1-[(4-aminophenyl)methyl]-4-piperidyl]-N-methyl-carbamate (0.750 g, 1.80 mmol, 69.12% yield). LC-MS (ES⁺): m/z 320.44 [M+H]⁺.

### Step-3:

Tert-butyl N-[1-[(4-aminophenyl)methyl]-4-piperidyl]-N-methyl-carbamate (1 g, 3.13 mmol) was dissolved in DMF (40 mL) and 3-bromopiperidine-2,6-dione (1.80 g, 9.39 mmol)and sodium bicarbonate (788.97 mg, 9.39 mmol) were added. The reaction mixture was heated to 100° C. for 16 hours. The reaction progress was monitored by TLC which showed consumption of starting material. The reaction mixture was then quenched with water, extracted with ethyl acetate, and the organic layer was washed with brine, dried over anhydrous Na₂SO₄ and concentrated under reduced pressure. The crude compound was purified by reverse phase prep-HPLC to afford tert-butyl N-[1-[[4-[(2,6-dioxo-3-piperidyl)amino]phenyl]methyl]-4-piperidyl]-N-methyl-carbamate (0.45 g, 877.97 μmol, 28.05% yield). LC-MS (ES⁺): m/z 431.32 [M+H]⁺.

### Step-4:

To a stirred solution of tert-butyl N-[1-[[4-[(2,6-dioxo-3-piperidyl)amino]phenyl]methyl]-4-piperidyl]-N-methyl-carbamate (0.15 g, 348.40 μmol) in DCM (5 mL) at 0° C. was added TFA (1.48 g, 12.98 mmol, 1 mL) over 5 minutes. The reaction mixture was stirred at 25° C. for 4 hours, and the reaction monitored by TLC (10% methanol in DCM, R_{f} value: 0.2). After reaction completion, the mixture was concentrated and co-distilled with toluene (10 ml) and diethyl ether (2×50 ml) to give a crude compound that was purified by prep-HPLC to afford 3-[4-[[4-(methylamino)-1-piperidyl]methyl]anilino]piperidine-2,6-dione (0.022 g, 62.48 μmol, 17.93% yield) as an off-white solid. LC-MS (ES⁺): m/z 330.95 [M+H]⁺.

### Synthesis of 3-[3-[[4-(methylamino)-1-piperidyl]methyl]anilino]piperidine-2,6-dione

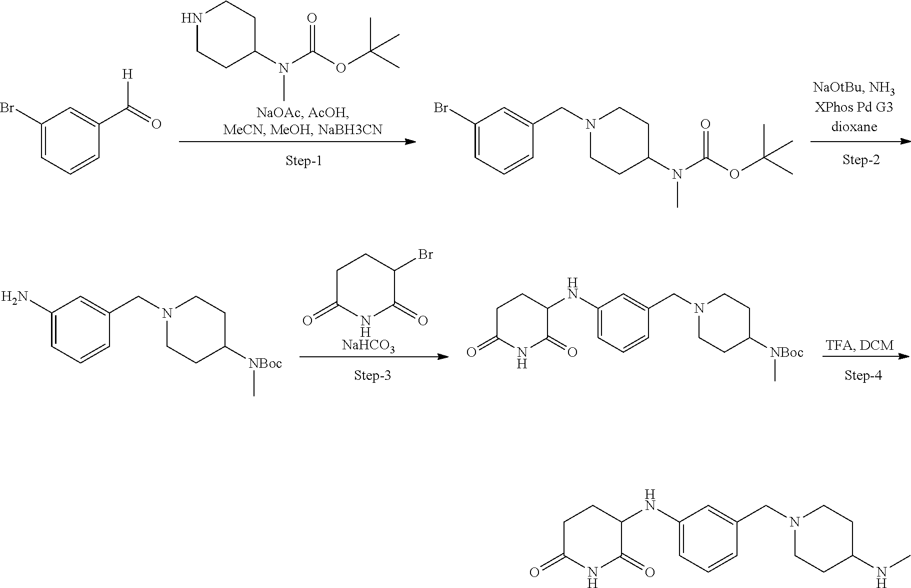

### Step-1:

To a stirred solution of 3-bromo benzaldehyde (5 g, 27.15 mmol) in a mixture of MeCN:MeOH (1:1 ratio, 20 mL) was added tert-butyl methyl(piperidin-4-yl)carbamate (6.95 g, 32.58 mmol) followed by sodium acetate (6.68 g, 81.45 mmol), and catalytic acetic acid (0.1 mL). The reaction was stirred at 100° C. for 3 hours. After 3 hours, the reaction mixture was cooled to 0° C. and sodium cyanobohydride (1.68 g, 27.15 mmol) added portion-wise and allowed to stir at room temperature for 16 hours. After complete consumption of the starting material, the reaction mixture was quenched with cold water. The solvent was evaporated under reduced pressure, diluted with water, and extracted with ethyl acetate. The combined organic layer was washed with brine solution, dried over anhydrous sodium sulfate, filtered, and concentrated to get crude compound which was purified using column chromatography (silica gel 100-200 mesh, EtOAc and pet ether) to afford tert-butyl (1-(3-bromobenzyl)piperidin-4-yl)(methyl)carbamate formic acid salt (5.5 g, 10.91 mmol, 40.20% yield). LC-MS (ES⁺): m/z 385.4 [M+H]⁺.

### Step-2:

In a sealed tube, a solution of tert-butyl (1-(3-bromobenzyl)piperidin-4-yl)(methyl)carbamate (3 g, 7.85 mmol) in 1,4-dioxane (20 mL) was added NaO^{t}Bu (2.26 g, 23.54 mmol). It was purged with ammonia gas for 20 minutes, followed by the addition of XPhos Pd G3 (1.25 g, 1.57 mmol). The resulting reaction mixture was heated and stirred at 90° C. for 16 hours. The progress of reaction was monitored by LC-MS and thin layer chromatography. After complete consumption of the starting material, the reaction mixture was filtered and the filtrate was concentrated to dryness. The crude compound was diluted with water and extracted with ethyl acetate. The combined organic layer was washed with brine solution, dried over anhydrous sodium sulfate, filtered, and concentrated to get tert-butyl 2-[1-[(3-aminophenyl)methyl]-4-piperidyl]propanoate (2 g, 1.24 mmol, 15.82% yield). LC-MS (ES⁺): m/z 320.38 [M+H]⁺.

### Step-3:

To a stirred solution of tert-butyl N-[1-[(3-aminophenyl)methyl]-4-piperidyl]-N-methyl-carbamate (2.0 g, 6.28 mmol) in DMF (20 mL) was added NaHCO₃ (1.58 g, 18.84 mmol) and the solution was purged with argon gas for 15 minutes. Then 3-bromopiperidine-2,6-dione (3.62 g, 18.84 mmol) was added and the resulting reaction mixture was heated at 90° C. with stirring for 16 hours. The progress of reaction was monitored by LC-MS and thin layer chromatography. After complete consumption of the starting material, the reaction mixture was concentrated to dryness and purified by prep-HPLC to afford tert-butyl (1-(3-((2,6-dioxopiperidin-3-yl)amino)benzyl)piperidin-4-yl)(methyl) carbamate formic acid salt (0.4 g, 792.17 μmol, 12.61% yield). LC-MS (ES⁺): m/z 320.38 [M+H]⁺.

### Step-4:

A stirred solution of tert-butyl (1-(3-((2,6-dioxopiperidin-3-yl)amino)benzyl) piperidin-4-yl)(methyl) carbamate TFA salt (0.03 g, 55.09 μmol) in DCM (3 mL) was cooled to 0° C. and TFA (444.00 mg, 3.89 mmol, 0.3 mL) was added. The reaction mixture was stirred at room temperature for 2 hours. The progress of reaction was monitored by LC-MS and thin layer chromatography (10% MeOH in DCM. R_{f} value: 0.3). After complete consumption of the starting material, the reaction mixture was concentrated to dryness to afford 3-[3-[[4-(methylamino)-1-piperidyl]methyl]anilino]piperidine-2,6-dione TFA salt (0.023 g, 42.36 μmol, 76.89% yield) as a light red solid. LC-MS (ES⁺): m/z 331.51 [M+H]⁺.

### Synthesis of 3-[3-[4-(methylamino)-1-piperidyl]anilino]piperidine-2,6-dione

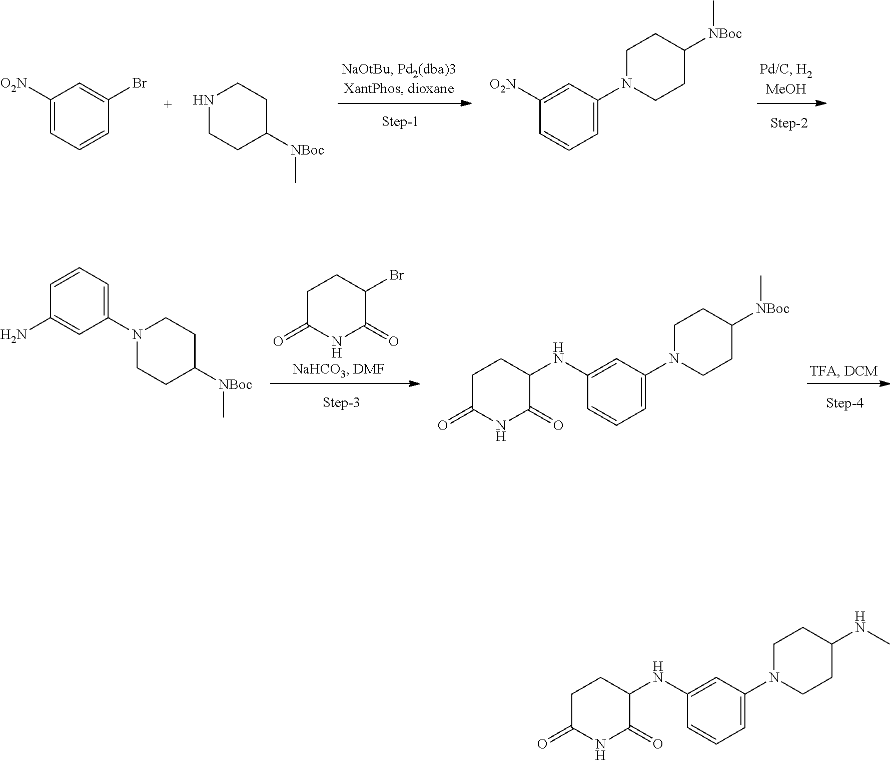

### Step-1:

To a stirred solution of tert-butyl N-methyl-N-(4-piperidyl)carbamate (5.30 g, 24.75 mmol) in dioxane (30 mL) were added sodium tert-butoxide (4.76 g, 49.50 mmol), tris(dibenzylideneacetone)dipalladium(O) (1.13 g, 1.12 mmol), 4,5-Bis(diphenylphosphino)-9,9-dimethylxanthene (286.44 mg, 495.04 μmol). The reaction was stirred for 15 minutes before adding 1-bromo-3-nitro-benzene (5.0 g, 24.75 mmol, 52.52 μL). The reaction mixture stirred at 100° C. for 16 hours while monitoring by TLC (Mobile phase: 50% EtOAc:Pet ether; R_{f} (Product): 0.5). After completion, the reaction mixture was quenched with ice and the precipitated solid was filtered and dried under vacuum to afford tert-butyl N-methyl-N-[1-(3-nitrophenyl)-4-piperidyl]carbamate (5.0 g, 14.61 mmol, 59.02% yield) as a yellow solid. LC-MS (ES⁺): m/z 336.2 [M+H]⁺.

### Step-2:

To a stirred solution of tert-butyl N-methyl-N-[1-(3-nitrophenyl)-4-piperidyl]carbamate (5.0 g, 14.91 mmol) in methanol (50 mL) was added palladium on carbon (5.00 g, 46.98 mmol) and the reaction mixture stirred at room temperature for 16 hours while monitoring by TLC (Mobile phase: 50% EtoAc:Pet ether. R_{f} (Product): 0.5). After completion, the reaction mixture was filtered through celite, and the organic layer was concentrated under reduced pressure to get the crude product which was purified by column chromatography (100 to 200 mesh silica gel, 0 to 50% EtOAc in pet ether) to afford tert-butyl N-[1-(3-aminophenyl)-4-piperidyl]-N-methyl-carbamate (3.0 g, 9.53 mmol, 63.91% yield) as a yellow solid. LC-MS (ES⁺): m/z 306.18 [M+H]⁺.

### Step-3:

To a stirred solution of tert-butyl N-[1-(3-aminophenyl)-4-piperidyl]-N-methyl-carbamate (2.5 g, 8.19 mmol) and 3-bromopiperidine-2,6-dione (3.14 g, 16.37 mmol) in DMF (3 mL) was added sodium bicarbonate (2.75 g, 32.74 mmol). The reaction mixture was stirred at 80° C. for 16 hours while monitoring by TLC. The reaction mixture was cooled to room temperature, diluted with water, and extracted with ethyl acetate. The organic phase was dried over sodium sulfate, filtered, and concentrated under reduced pressure to get crude product which was purified by column chromatography (100 to 200 mesh silica gel, 0 to 50% EtOAc in pet ether) to afford tert-butyl N-[1-[3-[(2,6-dioxo-3-piperidyl)amino]phenyl]-4-piperidyl]-N-methyl-carbamate (1.3 g, 2.90 mmol, 35.46% yield) as a yellow colored gummy liquid. LC-MS (ES⁺): m/z 417.53 [M+H]⁺.

### Step-4:

To a stirred solution of tert-butyl N-[1-[3-[(2,6-dioxo-3-piperidyl)amino]phenyl]-4-piperidyl]-N-methyl-carbamate (80 mg, 192.07 μmol) in DCM (5 mL) was added TFA (131.40 mg, 1.15 mmol, 88.79 μL) at 0° C. and stirred at room temperature for 2 hours, while monitoring by TLC. The reaction mixture was concentrated under reduced pressure and the residue triturated with diethyl ether (2×100 mL). The precipitated solid was filtered and dried under vacuum to afford 3-[3-[4-(methylamino)-1-piperidyl]anilino]piperidine-2,6-dione TFA salt (72 mg, 159.15 μmol, 82.86% yield) as a blue solid. LC-MS (ES⁺): m/z 317.52 [M+H]⁺.

### Synthesis of 3-[3-[4-(methylamino)-1-piperidyl]phenyl]piperidine-2,6-dione

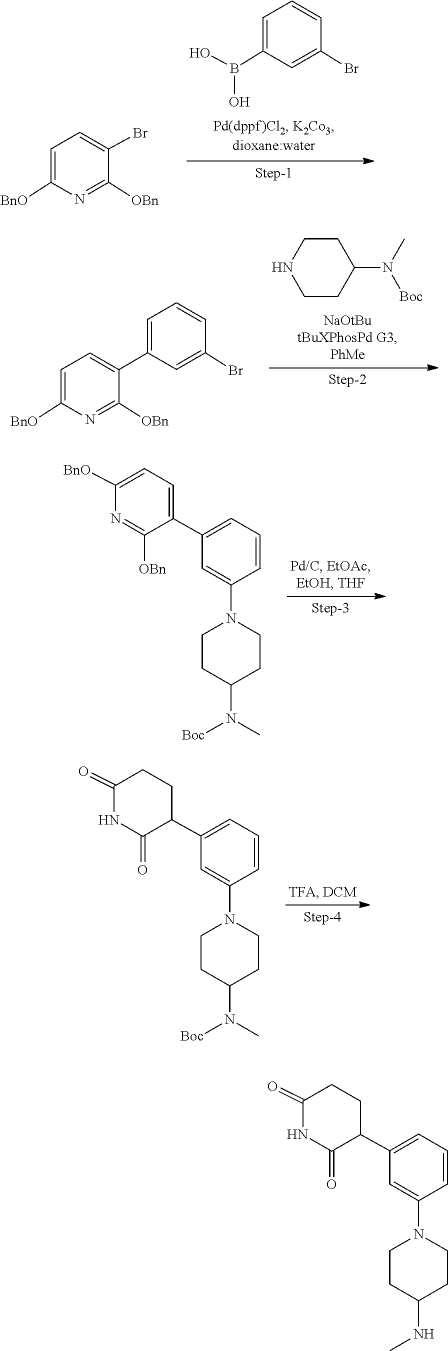

### Step-1:

A solution of (3-bromophenyl)boronic acid (1 g, 4.98 mmol), 2,6-dibenzyloxy-3-iodo-pyridine (2.08 g, 4.98 mmol), potassium carbonate (2.06 g, 14.94 mmol), cyclopentyl(diphenyl)phosphane;dichloropalladium;iron (364.35 mg, 497.94 μmol) and 2,6-dibenzyloxy-3-iodo-pyridine (2.08 g, 4.98 mmol) in dioxane:water (4:1 ratio 5 mL) was stirred for 16 hours at 90° C. The progress of the reaction was monitored by LC-MS. After completion of the reaction, the reaction mixture was diluted with cold water and extracted with ethyl acetate. The combined organic layer was washed with water, brine, dried over anhydrous Na₂SO₄, filtered, and concentrated under reduced pressure to give compound 2,6-dibenzyloxy-3-(3-bromophenyl)pyridine (1.500 g, 1.41 mmol, 28.35% yield). LCMS (ES⁺): m/z 446.2 [M+H]⁺.

### Step-2:

A solution of 2,6-dibenzyloxy-3-(3-bromophenyl)pyridine (1.3 g, 2.91 mmol), tert-butyl N-methyl-N-(4-piperidyl)carbamate (749.02 mg, 3.50 mmol), sodium tert-butoxide (559.82 mg, 5.83 mmol) and tBuXPhos Pd G3 (462.52 mg, 582.52 μmol) in toluene (15 mL) was stirred for 16 hours at 100° C. The reaction mixture was concentrated under reduced pressure, diluted with cold water and extracted with ethyl acetate. The combined organic layer was washed with water, brine, dried over anhydrous Na₂SO₄, filtered, and concentrated under reduced pressure to afford tert-butyl N-[1-[3-(2,6-dibenzyloxy-3-pyridyl)phenyl]-4-piperidyl]-N-methyl-carbamate (0.9 g, 947.00 μmol, 32.51% yield). LCMS (ES⁺): m/z 580.3 [M+H]⁺.

### Step-3:

To a stirred solution of tert-butyl N-[1-[3-(2,6-dibenzyloxy-3-pyridyl)phenyl]-4-piperidyl]-N-methyl-carbamate (1.8 g, 3.10 mmol) in a mixture of ethyl acetate: ethanol THF=1:5:4 (30 mL) was added 10% palladium on carbon (type 487, 1.8 g). Then the reaction mixture was stirred under H₂ (1 atm pressure) for 16 hours. The reaction mixture was passed through celite bed, then washed with methanol and concentrated under reduced pressure to obtain the desired crude compound. The crude material was purified by reverse phase column chromatography (Column/dimensions: X-SELECT C18 (19×250×5 um) Mobile phase A: 0.1% FA in water (aq) Mobile phase B: ACN (org) Gradient (Time/% B): 0/20,2/20,10/50,15/50,15.1/98,18/98,18.1/20,21/20. Flow rate: 16 ml/min. Solubility: ACN+THF+WATER) to afford compound tert-butyl N-[1-[3-(2,6-dioxo-3-piperidyl)phenyl]-4-piperidyl]-N-methyl-carbamate (1.00 g, 2.48 mmol, 79.92% yield). LCMS (ES⁻): m/z 400.3 [M−H]⁺.

### Step-4:

A solution of tert-butyl N-[1-[3-(2,6-dioxo-3-piperidyl)phenyl]-4-piperidyl]-N-methyl-carbamate (0.040 g, 99.63 μmol) and 20% 2,2,2-trifluoroacetic acid (11.36 mg, 99.63 μmol, 7.68 μL) in DCM (1 mL) was stirred for 4 hours at 0° C., then at room temperature. The progress of the reaction was monitored by LC-MS. After completion of the reaction, the reaction mixture was concentrated under reduced pressure and triturated with diethyl ether to afford 3-[3-[4-(methylamino)-1-piperidyl]phenyl]piperidine-2,6-dione TFA salt (27 mg, 64.16 μmol, 64.40% yield). LCMS (ES⁺): m/z 302.3 [M+H]⁺.

### Synthesis of 3-[4-[4-(methylamino)-1-piperidyl]phenyl]piperidine-2,6-dione

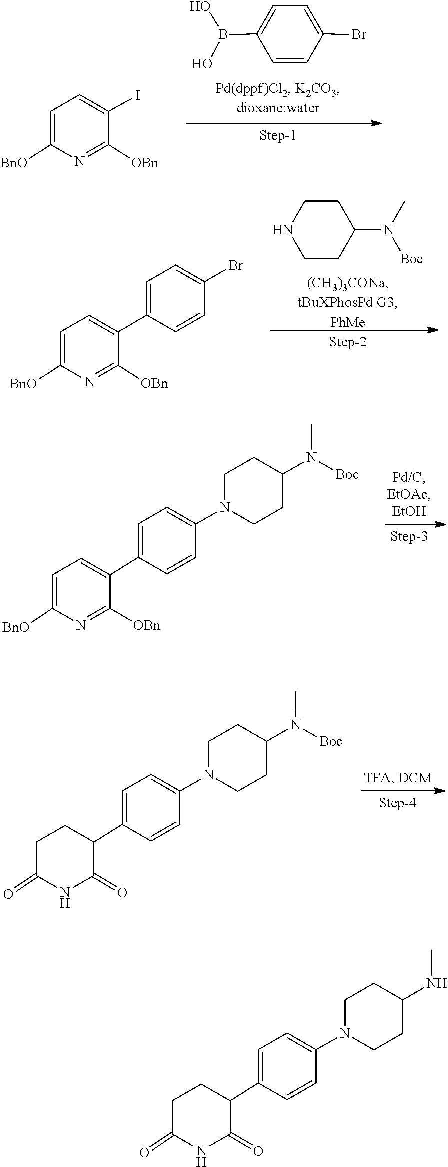

### Step-1:

In a seal tube, a solution of (4-bromophenyl)boronic acid (4 g, 19.92 mmol) and 2,6-dibenzyloxy-3-iodo-pyridine (8.31 g, 19.92 mmol) in dioxane and water (20 mL) was added potassium carbonate (8.25 g, 59.75 mmol). The reaction mixture was purged with argon for 20 minutes before Pd(dppf)Cl₂ (1.46 g, 1.99 mmol) was added and the reaction was stirred for 16 hours at 90° C., The reaction progress was monitored by LC-MS. After completion of the reaction, the reaction mixture was filtered and concentrated. It was then diluted with water and extracted with ethyl acetate. The organic layer was dried over anhydrous sodium sulfate and concentrated under reduced pressure to obtain the crude product, which was purified by column chromatography (60-120 mesh silica gel, 0-4% ethyl acetate in pet ether) to afford 2,6-dibenzyloxy-3-(4-bromophenyl)pyridine formic acid salt (7 g, 9.93 mmol, 49.83% yield) as an off-white solid. LCMS (ES⁺): m/z 446.1 [M+H]⁺.

### Step-2:

To a stirred solution of tert-butyl N-methyl-N-(4-piperidyl)carbamate TFA salt (1.47 g, 4.48 mmol) in toluene (20 mL) was added (CH₃)₃CONa (861.24 mg). After 10 minutes, 2,6-dibenzyloxy-3-(4-bromophenyl)pyridine (2 g, 4.48 mmol) was added and the resulting reaction mixture was stirred for 16 hours with heating. Progress of the reaction was monitored by LC-MS. The reaction crude was filtered and concentrated. The crude mixture was diluted in ethyl acetate and washed with water. The organic layer was dried over sodium sulfate and concentrated under reduced pressure. The crude product was purified by column chromatography to afford tert-butyl N-[1-[4-(2,6-dibenzyloxy-3-pyridyl)phenyl]-4-piperidyl]-N-methyl-carbamate (3 g, 3.83 mmol, 85.45% yield). LCMS (ES⁺): m/z 581.00 [M+H]⁺.

### Step-3:

To a stirred solution of tert-butyl N-[1-[4-(2,6-dibenzyloxy-3-pyridyl)phenyl]-4-piperidyl]-N-methyl-carbamate (3 g, 5.17 mmol) in EtOAc (10 mL), and EtOH (10 mL) was added Pd/C (3.14 g, 25.87 mmol) under hydrogen atmosphere. The resulting reaction mixture was stirred at room temperature for 16 hours. Progress of the reaction was monitored by LC-MS. After consumption of the starting material, the resulted crude was filtered and concentrated in vacuo. The crude product was purified by prep-HPLC (Column/dimensions: X-BRIDGE PHENYL-C18 (19*250*5 um), Mobile phase A: 5 mM ammonium acetate in water (aq), Mobile phase B: ACN (org)) to afford the compound tert-butyl N-[1-[4-(2,6-dioxo-3-piperidyl)phenyl]-4-piperidyl]-N-methyl-carbamate (1.7 g, 4.21 mmol, 81.37% yield). LCMS (ES⁺): m/z 402.5 [M+H]⁺.

### Step-4:

To a solution of tert-butyl N-[1-[4-(2,6-dioxo-3-piperidyl)phenyl]-4-piperidyl]-N-methyl-carbamate formic acid salt (0.05 g, 111.73 μmol) in DCM (2 mL) at 0° C. was added TFA (12.74 mg, 111.73 μmol, 8.61 μL) and the reaction mixture was stirred at room temperature for 2 hours. The reaction mixture was concentrated in vacuo to get the crude product, which was triturated with diethyl ether (5 mL) to afford 3-[4-[4-(methylamino)-1-piperidyl]phenyl]piperidine-2,6-dione formic acid salt (0.03 g, 84.15 μmol, 75.32% yield) as a light brown solid. LCMS (ES⁺): m/z 302.5 [M+H]⁺.

### Synthesis of 3-[4-[3,3-difluoro-4-(methylamino)-1-piperidyl]phenyl]piperidine-2,6-dione

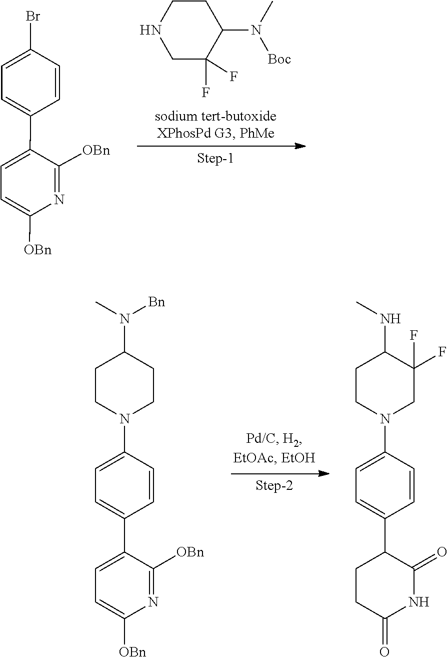 [SMILES: Brc1ccc(-c2ccc(OCc3ccccc3)nc2OCc2ccccc2)cc1 ; CN(C(=O)OC(C)(C)C)C1CCNCC1(F)F ; CN(Cc1ccccc1)C1CCN(c2ccc(-c3ccc(OCc4ccccc4)nc3OCc3ccccc3)cc2)CC1 ; CNC1CCN(c2ccc(C3CCC(=O)NC3=O)cc2)CC1(F)F]

### Step-1:

In a sealed tube, to the stirred solution of 2,6-dibenzyloxy-3-(4-bromophenyl)pyridine (1 g, 2.24 mmol) in toluene (10 mL) was added sodium tert-butoxide (645.93 mg, 6.72 mmol). After 10 minutes, N-benzyl-3,3-difluoro-N-methyl-piperidin-4-amine (646.04 mg, 2.69 mmol) was added, and the resulting reaction mixture was stirred for 16 hours at 100° C. Progress of the reaction was monitored by LC-MS. The reaction mixture was filtered and concentrated in vacuo. The residue was diluted with water (50 mL) and extracted with ethyl acetate (50 ml). The organic layer was dried over sodium sulfate and concentrated under reduced pressure. The crude product was purified by reverse-phase column chromatography using 80% acetonitrile (200 mL) and water (300 mL) to afford N-benzyl-1-[4-(2,6-dibenzyloxy-3-pyridyl)phenyl]-3,3-difluoro-N-methyl-piperidin-4-amine (0.4 g, 614.15 μmol, 27.41% yield). LCMS (ES⁺): m/z 606.5[M+H]⁺.

### Step-2:

A stirred solution of N-benzyl-1-[4-(2,6-dibenzyloxy-3-pyridyl)phenyl]-3,3-difluoro-N-methyl-piperidin-4-amine (1 g, 1.65 mmol) in EtOH (7 mL) and ethyl acetate (7 mL) was degassed with argon for 10 minutes. Palladium on carbon (1.00 g, 8.25 mmol) was added to the reaction mixture and it was stirred for 16 hours at room temperature under a H₂-balloon. Upon completion of the reaction, it was filtered through celite bed, washed with EtOH and EtOAc. The filtrate was evaporated under reduced pressure to give 3-[4-[3,3-difluoro-4-(methylamino)-1-piperidyl]phenyl]piperidine-2,6-dione TFA salt (0.45 g, 968.22 μmol, 58.65% yield) as a dark green solid. LCMS (ES⁺): m/z 338.5[M+H]⁺.

### Synthesis of 2-[1-[4-[(2,6-dioxo-3-piperidyl)amino]phenyl]-4-hydroxy-4-piperidyl]acetic acid

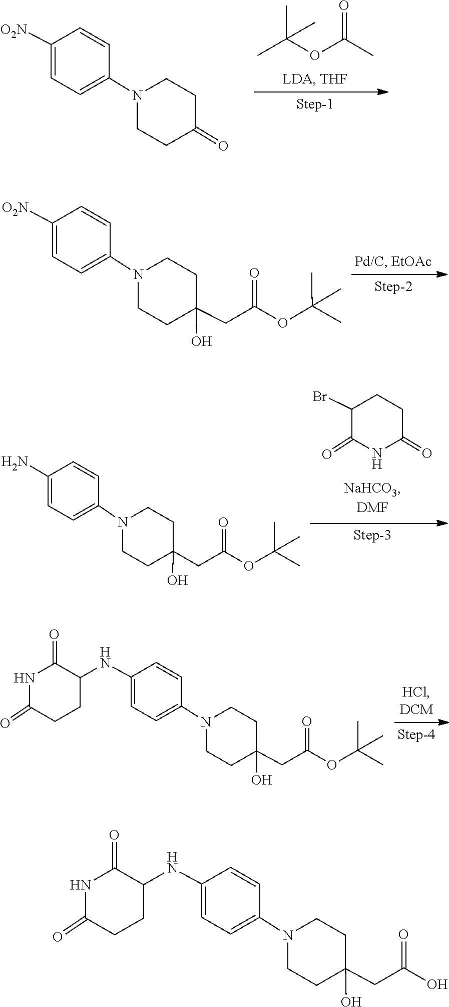

### Step-1:

Into a 500 mL multi neck round bottom flask containing a well stirred solution of tert-butyl acetate (2.64 g, 22.70 mmol, 3.06 mL) in anhydrous THE (75 mL) was added (diisopropylamino)lithium (2 M, 22.70 mL) at −78° C. under inert atmosphere. Then the resulting contents were stirred at −78° C. for 30 minutes. Later, hydroxy-oxo-[4-(4-oxo-1-piperidyl)phenyl]ammonium (5.02 g, 22.70 mmol) in THF (50 mL) was added to the reaction mixture at −78° C. and the reaction was allowed warm to −20° C. and stirred for 3 hours at the same temperature. After consumption of the starting material as indicated by TLC, the reaction was quenched with saturated ammonium chloride (100 mL). Reaction mixture was partitioned between ethyl acetate (250 mL) and water (200 mL). The organic layer was separated, washed with brine solution (100 mL) and dried over anhydrous sodium sulfate, and filtered. The filtrate was concentrated under reduced pressure to get the crude which was purified by flash column chromatography (silica gel 60-120 mesh, 0-50% EtOAc/n-hexane) to afford [4-[4-(2-tert-butoxy-2-oxo-ethyl)-4-hydroxy-1-piperidyl]phenyl]-hydroxy-oxo-ammonium (3 g, 6.68 mmol, 29.41% yield) as a yellow colored solid. LC-MS (ES⁺): m/z 337.4 [M+H]⁺.

### Step-2:

Into a 100 mL single neck round bottom flask containing a well stirred solution of [4-[4-(2-tert-butoxy-2-oxo-ethyl)-4-hydroxy-1-piperidyl]phenyl]-hydroxy-oxo-ammonium (3 g, 8.89 mmol) in ethyl acetate (30 mL) was added 10% palladium on activated carbon (50% wet with water) (946.26 mg, 8.89 mmol) under inert atmosphere at room temperature. Later, the reaction was stirred under hydrogen atmosphere for 16 hours at room temperature. After consumption of the starting material by TLC, the reaction mixture was filtered through a pad of celite, and the celite pad was washed with ethyl acetate (300 mL). The filtrate was concentrated under reduced pressure to give the crude tert-butyl 2-[1-(4-aminophenyl)-4-hydroxy-4-piperidyl]acetate (2.5 g, 8.04 mmol, 90.42% yield) as an off white solid. LCMS (ES⁺): m/z 307.2 [M+H]⁺.

### Step-3:

Into a 100 mL sealed tube containing a well stirred solution of 3-bromopiperidine-2,6-dione (1.57 g, 8.16 mmol) and tert-butyl 2-[1-(4-aminophenyl)-4-hydroxy-4-piperidyl]acetate (2.5 g, 8.16 mmol) in DMF (30 mL) was added sodium bicarbonate (2.06 g, 24.48 mmol) at ambient temperature under nitrogen atmosphere. Then the reaction was heated up to 70° C. for 16 hours. After consumption of the starting material as indicated by LCMS, the reaction mixture was poured into cold water (150 mL). Reaction mixture was partitioned between ethyl acetate (350 mL) and water (100 mL). The organic layer was separated, washed with brine solution (100 mL), dried over anhydrous sodium sulfate, and filtered. The filtrate was concentrated under reduced pressure to get the crude which was purified by flash column chromatography (silica gel 60-120 mesh, 0-100% EtOAc/n-hexane) to afford tert-butyl 2-[1-[4-[(2,6-dioxo-3-piperidyl)amino]phenyl]-4-hydroxy-4-piperidyl]acetate (2 g, 4.75 mmol, 58.24% yield) as a blue color solid. LC-MS(ES⁺): m/z 418.4 [M+H]⁺.

### Step-4:

Into a single neck round bottom flask containing a well stirred solution of tert-butyl 2-[1-[4-[(2,6-dioxo-3-piperidyl)amino]phenyl]-4-hydroxy-4-piperidyl]acetate (1 g, 2.40 mmol) in DCM (5 mL) was added hydrogen chloride solution in dioxane (4 M, 10 mL) at room temperature under nitrogen atmosphere and the resulting contents were stirred at the same temperature for 2 hours. After consumption of the starting material as indicated by TLC, the reaction mixture was concentrated under reduced pressure to get the crude which was azeotroped with toluene (2×15 mL) and triturated with MTBE (2×20 mL) to afford 2-[1-[4-[(2,6-dioxo-3-piperidyl)amino]phenyl]-4-hydroxy-4-piperidyl]acetic acid HCl salt (900 mg, 2.05 mmol, 85.62% yield) as a brown solid. LC-MS (ES⁺): m/z 361.2 [M+H]⁺.

### Synthesis of 2-[1-[2-chloro-4-[[(3S)-2,6-dioxo-3-piperidyl]amino]-6-fluoro-phenyl]-4-hydroxy-4-piperidyl]acetic acid

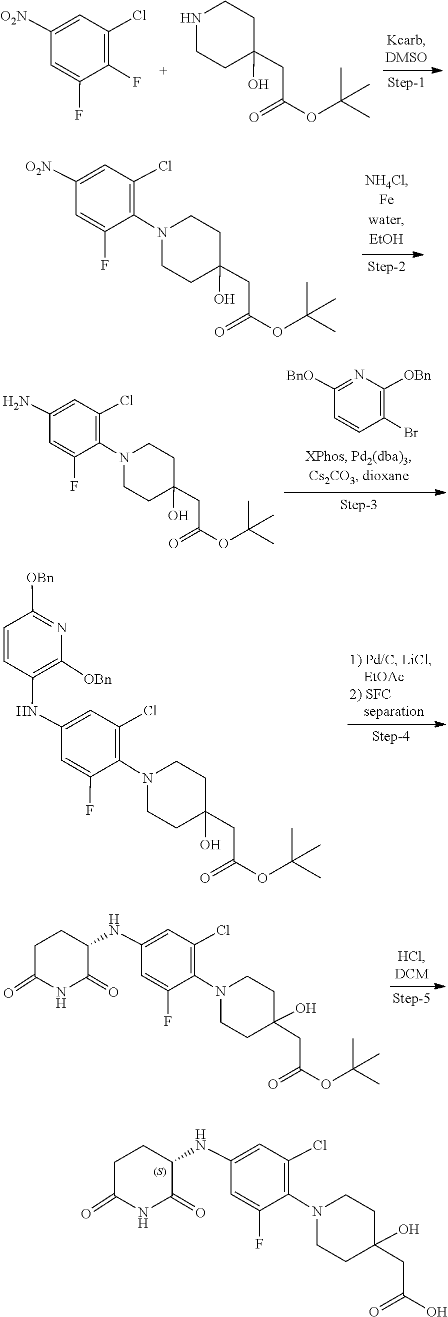

### Step-1:

To a solution of tert-butyl 2-(4-hydroxy-4-piperidyl)acetate (8 g, 37.16 mmol) and 1-chloro-2,3-difluoro-5-nitro-benzene (6.54 g, 33.78 mmol) in DMSO (80 mL) was added potassium carbonate (14.01 g, 101.34 mmol). The mixture was stirred at 110° C. for 1 hour. The reaction mixture was cooled to 20° C. and filtered. The filtrate was quenched with water (200 ml). The resulting mixture was filtered under vacuum and the filter cake was dried under vacuum to give a product tert-butyl 2-[1-(2-chloro-6-fluoro-4-nitro-phenyl)-4-hydroxy-4-piperidyl]acetate (13 g, 33.43 mmol, 98.97% yield) as a yellow solid. ¹H NMR (400 MHz, CDCl₃) δ=8.07-8.06 (m, 1H), 7.85-7.81 (dd, 1H), 3.85 (s, 1H), 3.65-3.52 (t, 2H), 3.24-3.12 (d, 2H), 2.46 (s, 2H), 1.78-1.71 (m, 4H), 1.49 (s, 9H).

### Step-2:

To the mixture of tert-butyl 2-[1-(2-chloro-6-fluoro-4-nitro-phenyl)-4-hydroxy-4-piperidyl]acetate (13 g, 33.43 mmol) in water (40 mL), ethanol (200 mL) were added ammonium chloride (8.94 g, 167.17 mmol, 5.84 mL) and iron powder (11.20 g, 200.61 mmol, 1.43 mL). The mixture was stirred at 90° C. for 1 hour. The reaction mixture was cooled to 25° C. and diatomite filtration was performed. The reaction mixture was concentrated under reduced pressure to remove EtOH. The residue was diluted with water (300 mL) and extracted with ethyl acetate (300 mL×2). The combined organic layers were washed with brine (200 mL×2), dried over anhydrous sodium sulfate, filtered and concentrated under reduced pressure. Tert-butyl 2-[1-(4-amino-2-chloro-6-fluoro-phenyl)-4-hydroxy-4-piperidyl]acetate (11 g, 30.65 mmol, 91.69% yield) was obtained as an orange oil. ¹H NMR (400 MHz, CDCl₃) δ=6.50-6.49 (m, 1H), 6.29-6.25 (dd, 1H), 3.72-3.61 (m, 3H), 3.43-3.32 (m, 2H), 3.89-3.78 (m, 2H), 2.45 (s, 2H), 1.75-1.72 (m, 4H), 1.48 (s, 9H).

### Step-3:

A stirred solution of tert-butyl 2-[1-(4-amino-2-chloro-6-fluoro-phenyl)-4-hydroxy-4-piperidyl]acetate (4.2 g, 11.70 mmol) and 2,6-dibenzyloxy-3-bromo-pyridine (6.50 g, 17.56 mmol) in dioxane (45 mL) was degassed with nitrogen for 15 minutes, then cesium carbonate (11.44 g, 35.11 mmol), XPhos (557.97 mg, 1.17 mmol) and Pd₂(dba)₃ (1.07 g, 1.17 mmol) were added at 25° C. The mixture was degassed with nitrogen for another 5 minutes, and was then heated to 100° C. for 16 hours under nitrogen atmosphere. The mixture was cooled to 25° C. and diluted with water (300 mL), and extracted with ethyl acetate (200 mL×2). The combined organic layers were washed with brine (300 mL×2), dried over with anhydrous sodium sulfate, filtered and concentrated under reduced pressure. The residue was purified by column chromatography (silica gel, petroleum ether/ethyl acetate=0/1 to 5/1) to afford tert-butyl 2-[1-[2-chloro-4-[(2,6-dibenzyloxy-3-pyridyl)amino]-6-fluoro-phenyl]-4-hydroxy-4-piperidyl]acetate (5.7 g, 8.79 mmol, 75.13% yield) as a yellow oil. LC-MS (ES⁺): m/z 648.2 [M+H]⁺.

### Step-4-1:

To the mixture of tert-butyl 2-[1-[2-chloro-4-[(2,6-dibenzyloxy-3-pyridyl)amino]-6-fluoro-phenyl]-4-hydroxy-4-piperidyl]acetate (5.6 g, 8.64 mmol) in ethyl acetate (57 mL) was added Pd/C (570 mg) and lithium chloride (732.55 mg, 17.28 mmol) under N₂ atmosphere. The mixture was stirred at 25° C. for 16 hours under H₂ (35 Psi). The mixture was filtered, and the filter cake was washed with ethyl acetate. The filtrate was concentrated to give a residue, which was purified by column chromatography (silica gel, petroleum ether/ethyl acetate=5/1 to 2/1) to afford tert-butyl 2-[1-[2-chloro-4-[(2,6-dioxo-3-piperidyl)amino]-6-fluoro-phenyl]-4-hydroxy-4-piperidyl]acetate (1.9 g, 4.04 mmol, 46.80% yield) as a blue solid. ¹H NMR (400 MHz, DMSO-d₆) δ=10.79 (s, 1H), 6.57 (s, 1H), 6.46-6.41 (dd, 1H), 6.21 (d, 1H), 4.43 (s, 1H), 4.35-4.30 (m, 1H), 3.31-3.16 (m, 2H), 2.78-2.63 (m, 3H), 2.54-2.52 (m, 1H), 2.33 (s, 2H), 2.12-1.98 (m, 1H), 1.91-1.68 (m, 3H), 1.63-1.54 (m, 2H), 1.41 (s, 9H). LC-MS (ES⁺): m/z 470.1 [M+H]⁺.

### Step-4-2:

Tert-butyl 2-[1-[2-chloro-4-[(2,6-dioxo-3-piperidyl)amino]-6-fluoro-phenyl]-4-hydroxy-4-piperidyl]acetate (2.2 g, 4.68 mmol) was purified by prep-SFC using the following conditions.

Sample preparation: add IPA and CH₂Cl₂ 100 ml into sample

Instrument: Waters 80Q

Mobile Phase:50% IPA (Neu) in supercritical CO₂

Flow Rate:70 g/min

Cycle Time:4.4 min, total time:550 min

Single injection volume:1.5 ml

Back Pressure:100 bar to keep the CO₂ in Supercritical flow

Compound tert-butyl 2-[1-[2-chloro-4-[[(3S)-2,6-dioxo-3-piperidyl]amino]-6-fluoro-phenyl]-4-hydroxy-4-piperidyl]acetate (900 mg, 1.84 mmol, 39.27% yield) was obtained as a blue solid and confirmed by HPLC and SFC.

Compound tert-butyl 2-[1-[2-chloro-4-[[(3R)-2,6-dioxo-3-piperidyl]amino]-6-fluoro-phenyl]-4-hydroxy-4-piperidyl]acetate (1 g, 2.13 mmol, 45.45% yield) was obtained as a blue solid.

### Step-5:

To a solution of tert-butyl 2-[1-[2-chloro-4-[[(3S)-2,6-dioxo-3-piperidyl]amino]-6-fluoro-phenyl]-4-hydroxy-4-piperidyl]acetate (0.25 g, 531.99 μmol) in DCM (3 mL) was added 4 M hydrochloric acid in 1,4 dioxane (3 mL). The mixture was stirred at 20° C. for 16 hours. After completion of the reaction, the reaction mixture was concentrated under reduced pressure and the residue was triturated with diethyl ether and filtered to give the product 2-[1-[2-chloro-4-[[(3S)-2,6-dioxo-3-piperidyl]amino]-6-fluoro-phenyl]-4-hydroxy-4-piperidyl]acetic acid HCl salt (240 mg, 522.33 μmol, 98.18% yield) as a blue solid. LC-MS (ES⁺): m/z 414.1 [M+H]⁺.

### Synthesis of 2-[1-[3-(2,6-dioxo-3-piperidyl)-1-methyl-indazol-6-yl]-4-hydroxy-4-piperidyl]acetic acid

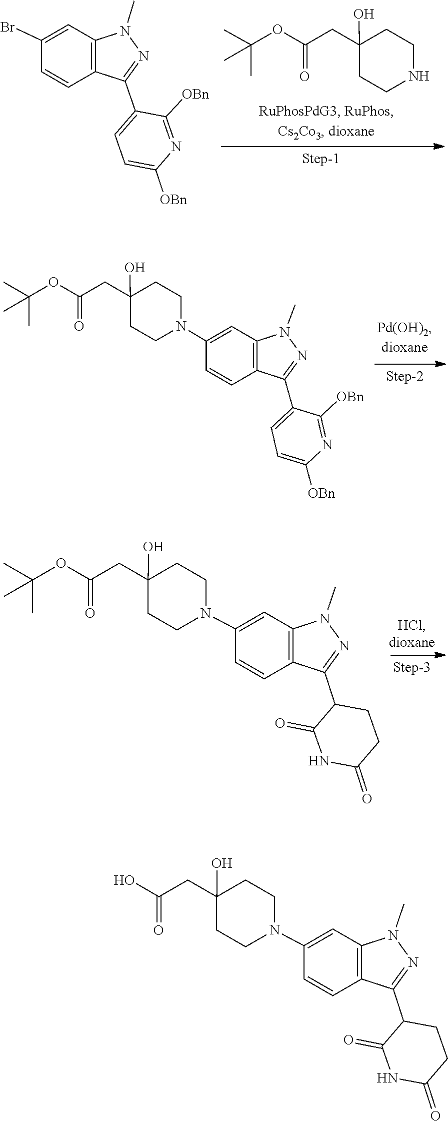 [SMILES: Cn1nc(-c2ccc(OCc3ccccc3)nc2OCc2ccccc2)c2ccc(Br)cc21 ; CC(C)(C)OC(=O)CC1(O)CCNCC1 ; Cn1nc(-c2ccc(OCc3ccccc3)nc2OCc2ccccc2)c2ccc(N3CCC(O)(CC(=O)OC(C)(C)C)CC3)cc21 ; Cn1nc(C2CCC(=O)NC2=O)c2ccc(N3CCC(O)(CC(=O)OC(C)(C)C)CC3)cc21 ; Cn1nc(C2CCC(=O)NC2=O)c2ccc(N3CCC(O)(CC(=O)O)CC3)cc21]

### Step-1:

In a sealed-tube, a solution of 6-bromo-3-(2,6-dibenzyloxy-3-pyridyl)-1-methyl-indazole (1.4 g, 2.80 mmol) in 1,4-dioxane (10.0 mL) was added tert-butyl 2-(4-hydroxy-4-piperidyl)acetate (602.34 mg, 2.80 mmol) and cesium carbonate (2.73 g, 8.39 mmol) with stirring at room temperature under nitrogen atmosphere. The reaction mixture was degassed with nitrogen for 15 minutes before RuPhos (130.56 mg, 279.78 μmol) and RuPhosPdG3 (234.00 mg, 279.78 μmol) were added to the reaction mixture. The mixture was degassed again with nitrogen for 5 minutes and heated to 100° C. for 2.5 hours. After completion of the reaction by TLC, the reaction mixture was diluted with ethyl acetate (50.0 mL), washed with water (20.0 ml) and brine solution (30.0 mL). The organic layer was dried over anhydrous sodium sulfate and concentrated under reduced pressure to give the crude product, which was purified by column chromatography (silica gel 100-200 mesh, 50% ethyl acetate in pet ether) to afford tert-butyl 2-[1-[3-(2,6-dibenzyloxy-3-pyridyl)-1-methyl-indazol-6-yl]-4-hydroxy-4-piperidyl]acetate (1.1 g, 1.64 mmol, 58.64% yield) as an off-white solid. LC-MS (ES⁺): m/z 635.2 [M+H]⁺.

### Step-2:

To a stirred solution of tert-butyl 2-[1-[3-(2,6-dibenzyloxy-3-pyridyl)-1-methyl-indazol-6-yl]-4-hydroxy-4-piperidyl]acetate (2.0 g, 3.15 mmol) in 1,4-dioxane (30 mL) purged with nitrogen gas, was added palladium hydroxide on carbon, 20 wt. % dry basis (442.48 mg, 3.15 mmol) and the reaction mixture was stirred under hydrogen atmosphere at room temperature for 16 hours. After completion of the reaction, the reaction mixture was filtered through celite bed, washed with ethyl acetate (200 mL) and concentrated under reduced pressure to get the crude product, which was purified by column chromatography (silica gel, 75% ethyl acetate in pet ether) to afford tert-butyl 2-[1-[3-(2,6-dioxo-3-piperidyl)-1-methyl-indazol-6-yl]-4-hydroxy-4-piperidyl]acetate (1.2 g, 2.59 mmol, 82.34% yield) as an off white solid. LC-MS (ES⁺): m/z 457.2 [M+H]⁺.

### Step-3:

To a stirred solution of tert-butyl 2-[1-[3-(2,6-dioxo-3-piperidyl)-1-methyl-indazol-6-yl]-4-hydroxy-4-piperidyl]acetate (1.2 g, 2.59 mmol) in 1,4-dioxane (15 mL) cooled to 0° C. was added 4.0 M hydrogen chloride solution in dioxane (648.58 mmol) dropwise and the reaction was stirred at room temperature for 50 hours. After completion of the reaction, the reaction mixture was concentrated, washed with hexane (100 mL) and dried to get the product 2-[1-[3-(2,6-dioxo-3-piperidyl)-1-methyl-indazol-6-yl]-4-hydroxy-4-piperidyl]acetic acid HCl salt (1.15 g, 2.16 mmol, 83.40% yield) as an off white solid. LC-MS (ES⁺): m/z 401.2 [M+H]⁺.

### Synthesis of 2-(1-(4-(2,6-dioxopiperidin-3-yl)-2,5-difluorophenyl)-4-hydroxypiperidin-4-yl)acetic acid

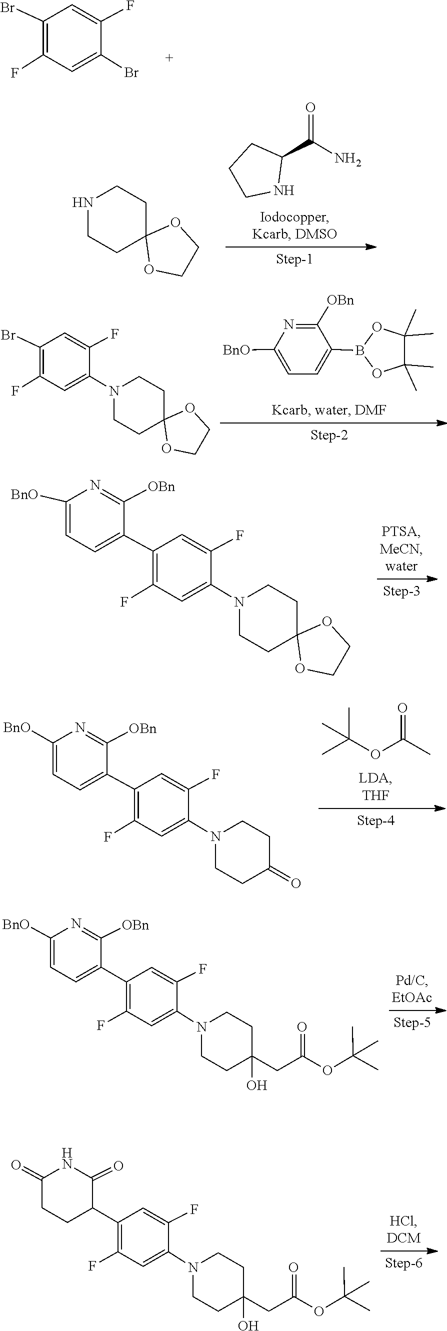

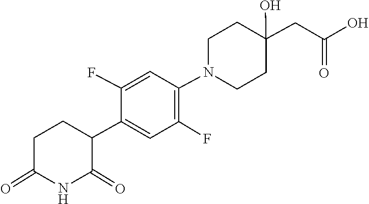 [SMILES: O=C(O)CC1(O)CCN(c2cc(F)c(C3CCC(=O)NC3=O)cc2F)CC1]

### Step-1:

A mixture of 1,4-dioxa-8-azaspiro[4.5]decane (20 g, 139.68 mmol, 17.86 mL), 1,4-dibromo-2,5-difluoro-benzene (113.93 g, 419.04 mmol), iodocopper (6.65 g, 34.92 mmol, 1.18 mL), potassium carbonate (57.92 g, 419.04 mmol) and (2S)-pyrrolidine-2-carboxamide (7.97 g, 69.84 mmol) in DMSO (120 mL) was degassed and purged with N₂ three times, and then the mixture was stirred at 60° C. for 6 hours under N₂ atmosphere. The reaction mixture was diluted with water (10 mL) and extracted with ethyl acetate (15 mL×3). The combined organic layers were washed with brine (10 mL×2), dried over anhydrous sodium sulfate, filtered, and concentrated under reduced pressure. The residue was purified by flash column chromatography (ISCO®; 20 g SepaFlash® Silica Flash Column, 0-10% ethyl acetate/petroleum ether as eluent at 50 mL/min). Compound 8-(4-bromo-2,5-difluoro-phenyl)-1,4-dioxa-8-azaspiro[4.5]decane (1.8 g, 4.31 mmol, 3.09% yield) was obtained as a white solid. LC-MS (ES⁺): m/z 334.0 [M+H]⁺.

### Step-2:

A mixture of 8-(4-bromo-2,5-difluoro-phenyl)-1,4-dioxa-8-azaspiro[4.5]decane (1.8 g, 5.39 mmol),2,6-bis(benzyloxy)-3-(4,4,5,5-tetramethyl-1,3,2-dioxaborolan-2-yl)pyridine (3.37 g, 8.08 mmol), cyclopentyl(diphenyl)phosphane; dichloropalladium; iron (394.15 mg, 538.67 μmol), K₂CO₃ (2.23 g, 16.16 mmol) in DMF (20 mL) and water (4 mL) was degassed and purged with N₂ three times. The mixture was stirred at 25° C. for 0.5 hour under N₂ atmosphere. Then the mixture was stirred at 80° C. for 16 hours under N₂ atmosphere. After the reaction was complete, the reaction mixture was diluted with water (10 mL) and extracted with ethyl acetate (20 mL×3). The combined organic layers were washed with CaCl₂ solution (20 mL) and brine (20 mL). It was then dried over anhydrous sodium sulfate, filtered, and concentrated under reduced pressure. The residue was purified by flash column chromatography (ISCO®; 40 g SepaFlash® Silica Flash Column, 0-50% ethyl acetate/petroleum ether at 60 mL/min). Compound 8-(4-(2,6-bis(benzyloxy)pyridin-3-yl)-2,5-difluorophenyl)-1,4-dioxa-8-azaspiro[4.5]decane (1.8 g, 2.46 mmol, 45.61% yield) was obtained as a white solid. LC-MS(ES⁺): m/z 545.2 [M+H]⁺.

### Step-3:

To a solution of 8-[4-(2,6-dibenzyloxy-3-pyridyl)-2,5-difluoro-phenyl]-1,4-dioxa-8-azaspiro[4.5]decane (1.0 g, 1.36 mmol) in acetone (90 mL) and water (21 mL) was added PTSA (935.97 mg, 5.44 mmol). The mixture was stirred at 50° C. for 16 hours. After completion. the reaction mixture was concentrated under reduced pressure to remove acetone and the residue was diluted with NaHCO₃ solution (30 mL) and extracted with ethyl acetate (30 mL×3). The combined organic layers were washed with brine (20 mL×1), dried over anhydrous sodium sulfate, filtered, and concentrated under reduced pressure. Compound 1-[4-(2,6-dibenzyloxy-3-pyridyl)-2,5-difluoro-phenyl]piperidin-4-one (1.03 g, 1.03 mmol, 75.72% yield) was obtained as a white solid. LC-MS (ES⁺): m/z 501.2 [M+H]⁺.

### Step-4:

To a solution of LDA (1 M, 3.02 mL) in THF (45 mL) was added tert-butyl acetate (336.27 mg, 2.89 mmol, 389.65 μL) dropwise at −70° C. After the addition, the mixture was stirred at −78° C. for 1 hour, then a solution of 1-[4-(2,6-dibenzyloxy-3-pyridyl)-2,5-difluoro-phenyl]piperidin-4-one (1.8 g, 2.52 mmol) in THE (45 mL) was added dropwise via a funnel. After 30 minutes at −70° C., the mixture was stirred at 20° C. for 1 hour. After completion of the reaction, the reaction mixture was quenched by addition of NH₄Cl solution (10 mL) and extracted with ethyl acetate (15 mL×3). The combined organic layers were washed with brine (15 mL×2), dried over anhydrous sodium sulfate, filtered, and concentrated under reduced pressure. The residue was purified by flash column chromatography (ISCO®; 40 g SepaFlash® Silica Flash Column, 0-40% ethyl acetate/petroleum ether as eluent at 50 mL/min). Tert-butyl 2-(1-(4-(2,6-bis(benzyloxy)pyridin-3-yl)-2,5-difluorophenyl)-4-hydroxypiperidin-4-yl)acetate (600 mg, 846.45 μmol, 33.63% yield) was obtained as a yellow solid. LC-MS (ES⁺): m/z 617.3 [M+H]⁺.

### Step-5:

To a solution of tert-butyl 2-[1-[4-(2,6-dibenzyloxy-3-pyridyl)-2,5-difluoro-phenyl]-4-hydroxy-4-piperidyl]acetate (0.6 g, 972.93 μmol) in methanol (5 mL) was added 10% Pd/C (590.83 mg, 486.47 μmol). The mixture was stirred at 25° C. for 16 hours. After completion of the reaction, the reaction mixture was concentrated under reduced pressure to remove methanol. The crude product tert-butyl 2-(1-(4-(2,6-dioxopiperidin-3-yl)-2,5-difluorophenyl)-4-hydroxypiperidin-4-yl)acetate (0.4 g, 912.28 μmol, 93.77% yield) was used in the next step without further purification. LC-MS(ES⁺): m/z 437.1 [M−H]⁺.

### Step-6:

To a solution of tert-butyl 2-(1-(4-(2,6-dioxopiperidin-3-yl)-2,5-difluorophenyl)-4-hydroxypiperidin-4-yl)acetate (0.4 g, 912.28 μmol) in DCM (2 mL) was added HCl (12 M, 760.23 μL). The mixture was stirred at 25° C. for 5 hours. The residue was purified by prep-HPLC (ACSWH-GX-U/Phenomenex Luna C18 150×40 mm×15 um; water (0.1% TFA)/ACN; 10-40% gradient; Time (min): 11). Compound 2-(1-(4-(2,6-dioxopiperidin-3-yl)-2,5-difluorophenyl)-4-hydroxypiperidin-4-yl)acetic acid (0.1 g, 238.77 μmol, 26.17% yield) was obtained as a white solid. ¹H NMR (400 MHz, DMSO-d₆) δ=12.53 (s, 1H), 10.85 (s, 1H), 7.10 (dd, J=7.2, 13.2 Hz, 1H), 6.85 (dd, J=7.2, 12.0 Hz, 1H), 4.43-4.12 (m, 1H), 4.09-3.87 (m, 2H), 3.15-2.95 (m, 3H), 2.78-2.65 (m, 1H), 2.53 (br d, J=3.6 Hz, 1H), 2.40 (s, 2H), 2.19 (dq, J=3.6, 13.0 Hz, 1H), 2.00-1.91 (m, 1H), 1.85-1.75 (m, 2H), 1.72-1.64 (m, 2H).

### Synthesis of 2-[1-[4-[(2,6-dioxo-3-piperidyl)amino]-2,5-difluoro-phenyl]-4-hydroxy-4-piperidyl]acetic acid

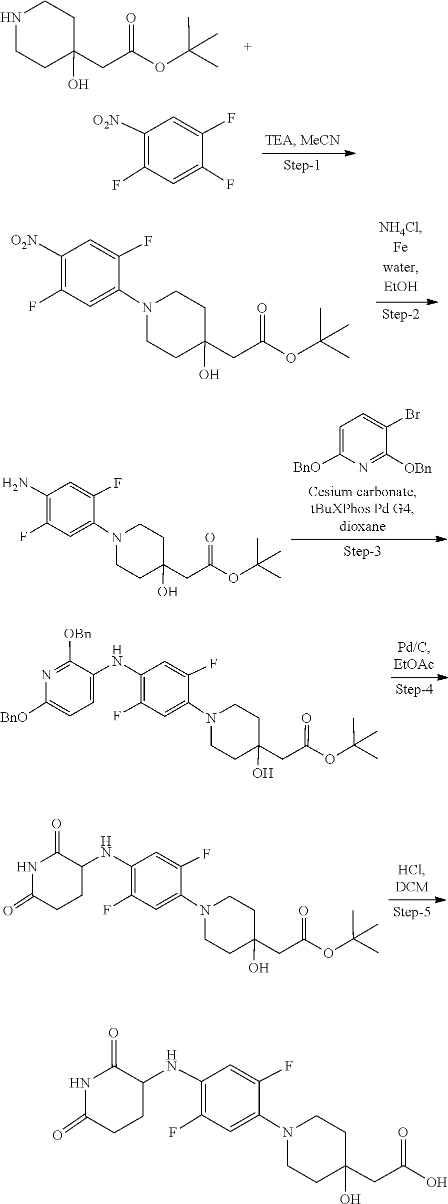

### Step-1:

To a solution of 1,2,4-trifluoro-5-nitro-benzene (4 g, 22.59 mmol, 2.60 mL) and tert-butyl 2-(4-hydroxy-4-piperidyl)acetate (4.86 g, 22.59 mmol) in acetonitrile (50 mL) was added TEA (85.72 mg, 847.07 μmol, 118.06 μL), then the mixture was stirred at 20° C. for 1 hour. After completion of the reaction, the mixture was concentrated under reduced pressure to give a residue, which was purified by column chromatography (silica gel, petroleum ether/ethyl acetate=1/0 to 1/1) to afford tert-butyl 2-[1-(2,5-difluoro-4-nitro-phenyl)-4-hydroxy-4-piperidyl]acetate (5 g, 13.32 mmol, 58.97% yield) as a yellow solid. LC-MS (ES⁺): m/z 373.2 [M+H]⁺.

### Step-2:

To a solution of tert-butyl 2-[1-(2,5-difluoro-4-nitro-phenyl)-4-hydroxy-4-piperidyl]acetate (2 g, 5.37 mmol) in ethanol (20 mL) and water (4 mL) was added iron (1.20 g, 21.48 mmol) and ammonium chloride (2.30 g, 42.97 mmol), then the mixture was stirred at 20° C. for 3 hours. After completion of the reaction, the mixture was filtered and concentrated under reduced pressure to give tert-butyl 2-[1-(4-amino-2,5-difluoro-phenyl)-4-hydroxy-4-piperidyl]acetate (1.5 g, 4.24 mmol, 78.95% yield) as a brown solid. LC-MS(ES⁺): m/z 343.2 [M+H]⁺.

### Step-3:

To a solution of 2,6-dibenzyloxy-3-bromo-pyridine (1.47 g, 3.98 mmol) and tert-butyl 2-[1-(4-amino-2,5-difluoro-phenyl)-4-hydroxy-4-piperidyl]acetate (1.5 g, 4.38 mmol) in dioxane (15 mL) was added cesium carbonate (3.89 g, 11.95 mmol) and tBuXPhos Pd G3 (316.79 mg, 398.28 μmol). Then the mixture was stirred at 90° C. for 16 hours under N₂ atmosphere. Upon completion, the reaction was quenched by water (30 mL), and then extracted with ethyl acetate (15 mL×3). The combined organic layers were washed with brine (10 mL×3), dried over anhydrous sodium sulfate, filtered, and concentrated under reduced pressure. The residue was purified by column chromatography (silica gel, petroleum ether/ethyl acetate=1/0 to 1/1) to afford tert-butyl 2-[1-[4-[(2,6-dibenzyloxy-3-pyridyl)amino]-2,5-difluoro-phenyl]-4-hydroxy-4-piperidyl]acetate (1.3 g, 1.95 mmol, 49.05% yield) as a brown oil. LC-MS(ES⁺): m/z 632.5[M+H]⁺.

### Step-4:

To a solution of tert-butyl 2-[1-[4-[(2,6-dibenzyloxy-3-pyridyl)amino]-2,5-difluoro-phenyl]-4-hydroxy-4-piperidyl]acetate (1.3 g, 2.06 mmol) in ethyl acetate (15 mL) was added 10% Pd (219.00 mg, 205.79 μmol), and the mixture was stirred at 20° C. for 16 hours under H₂ (15 psi). After completion of the reaction, the reaction mixture was filtered and concentrated under reduced pressure to give tert-butyl 2-[1-[4-[(2,6-dioxo-3-piperidyl)amino]-2,5-difluoro-phenyl]-4-hydroxy-4-piperidyl]acetate (0.8 g, 1.76 mmol, 85.72% yield) as a brown oil. LC-MS(ES⁺): m/z 454.3 [M+H]⁺.

### Step-5:

To a solution of tert-butyl 2-[1-[4-[(2,6-dioxo-3-piperidyl)amino]-2,5-difluoro-phenyl]-4-hydroxy-4-piperidyl]acetate (0.8 g, 1.76 mmol) in DCM (8 mL) was added hydrochloric acid (12 M, 1.47 mL) at 0° C., and the mixture was stirred at 25° C. for 1 hour. The reaction mixture was concentrated under reduced pressure to give a residue. The residue was diluted with acetonitrile (5 mL) and stirred at 25° C. for 15 minutes. It was then filtered and the filter cake was dried over vacuum to give 2-[1-[4-[(2,6-dioxo-3-piperidyl)amino]-2,5-difluoro-phenyl]-4-hydroxy-4-piperidyl]acetic acid HCl salt (600 mg, 1.38 mmol, 78.40% yield) as a purple solid. LC-MS(ES⁺): m/z 398.1 [M+H]⁺.

### Synthesis of 1-[2,5-dichloro-4-[(2,6-dioxo-3-piperidyl)amino]phenyl]-4-hydroxy-4-piperidyl]acetic acid

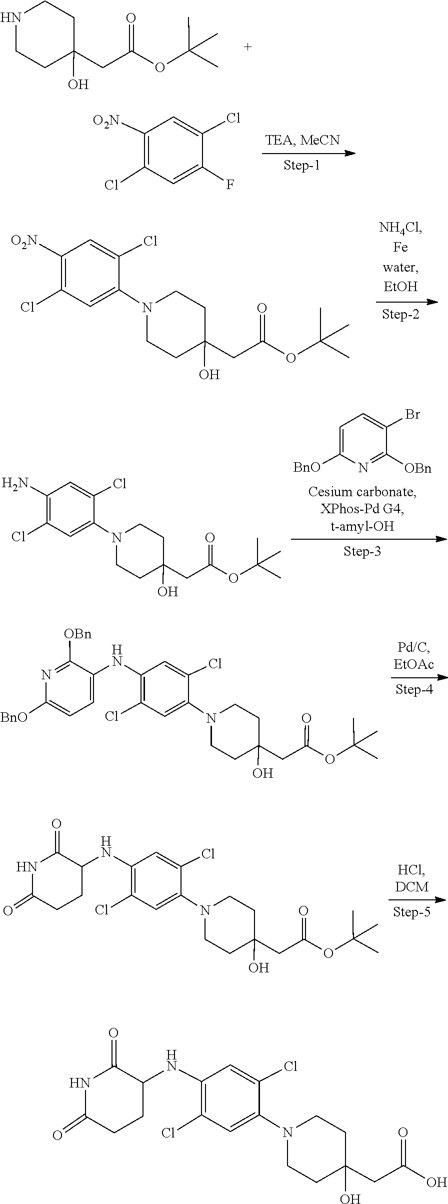

### Step-1:

To a solution of 1,4-dichloro-2-fluoro-5-nitro-benzene (2 g, 9.52 mmol, 1.30 mL) and tert-butyl 2-(4-hydroxy-4-piperidyl)acetate (2.26 g, 10.48 mmol) in acetonitrile (20 mL) was added TEA (1.45 g, 14.29 mmol, 1.99 mL). The mixture was stirred at 25° C. for 2 hours. Upon completion of the reaction, the reaction mixture was quenched by water (50 mL) and extracted with ethyl acetate (20 mL×3). The combined organic layers were washed with brine (5 mL×3), dried over Na₂SO₄, filtered, and concentrated under reduced pressure. The product was taken forward to the next step without purification. Compound tert-butyl 2-[1-(2,5-dichloro-4-nitro-phenyl)-4-hydroxy-4-piperidyl]acetate (3.5 g, 8.48 mmol, 89.02% yield) was obtained as a yellow solid. LC-MS (ES⁺): m/z 405.1 [M+H]⁺.

### Step-2:

To a solution of tert-butyl 2-[1-(2,5-dichloro-4-nitro-phenyl)-4-hydroxy-4-piperidyl]acetate (3.5 g, 8.64 mmol) in ethanol (30 mL) and water (6 mL) was added iron (1.93 g, 34.54 mmol, 245.46 μL). The mixture was stirred at 25° C. for 5 hours. The reaction mixture was filtered and concentrated under reduced pressure and extracted with ethyl acetate (10 mL×3). The combined organic layers were washed with brine (5 mL×3), dried over Na₂SO₄, filtered, and concentrated under reduced pressure to give tert-butyl 2-[1-(4-amino-2,5-dichloro-phenyl)-4-hydroxy-4-piperidyl]acetate (3 g, 7.89 mmol, 91.32% yield) as a yellow solid. LC-MS (ES⁺): m/z 375.1 [M+H]⁺.

### Step-3:

To a solution of tert-butyl 2-[1-(4-amino-2,5-dichloro-phenyl)-4-hydroxy-4-piperidyl]acetate (1 g, 2.66 mmol) and 2,6-dibenzyloxy-3-bromo-pyridine (1.18 g, 3.20 mmol) in t-Amyl-OH (10 mL) was added cesium carbonate (2.60 g, 7.99 mmol) and X-Phos-Pd G4 (229.28 mg, 266.46 μmol). The mixture was stirred at 90° C. for 16 hours. The reaction mixture was filtered and concentrated under reduced pressure, then extracted with ethyl acetate (10 mL×3). The combined organic layers were washed with brine (5 mL×3), dried over anhydrous sodium sulfate, filtered, and concentrated under reduced pressure. The residue was purified by column chromatography (silica gel, petroleum ether/ethyl acetate=1/0 to 1/1) to afford tert-butyl 2-[1-[2,5-dichloro-4-[(2,6-dibenzyloxy-3-pyridyl)amino]phenyl]-4-hydroxy-4-piperidyl]acetate (550 mg, 723.94 μmol, 27.17% yield) as a yellow oil. LC-MS (ES⁺): m/z 664.1 [M+H]⁺.

### Step-4:

To a solution of tert-butyl 2-[1-[2,5-dichloro-4-[(2,6-dibenzyloxy-3-pyridyl)amino]phenyl]-4-hydroxy-4-piperidyl]acetate (550 mg, 827.55 μmol) in ethyl acetate (6 mL) was added 10% Pd/C (50 mg), and the mixture was stirred at 25° C. for 6 hours under H₂ (15 psi). The reaction mixture was filtered and concentrated under reduced pressure. The material was taken forward crude. Compound tert-butyl 2-[1-[2,5-dichloro-4-[(2,6-dioxo-3-piperidyl)amino]phenyl]-4-hydroxy-4-piperidyl]acetate (150 mg, 245.36 μmol, 29.65% yield) was obtained as a black solid. LC-MS (ES⁺): m/z 486.2 [M+H]⁺.

### Step-5:

To a solution of tert-butyl 2-[1-[2,5-dichloro-4-[(2,6-dioxo-3-piperidyl)amino]phenyl]-4-hydroxy-4-piperidyl]acetate (150 mg, 308.40 μmol) in DCM (2 mL) was added hydrochloric acid (12 M, 257.00 μL) at 0° C., then the mixture was stirred at 25° C. for 1 hour. After completion, the reaction mixture was concentrated under reduced pressure to give a residue, which was purified by prep-HPLC (TFA condition) to give 1-[2,5-dichloro-4-[(2,6-dioxo-3-piperidyl)amino]phenyl]-4-hydroxy-4-piperidyl]acetic acid (90 mg, 180.51 μmol, 58.53% yield) as a black solid. LC-MS (ES⁺): m/z 429.9 [M+H]⁺.

### Synthesis of 2-[1-[4-[(2,6-dioxo-3-piperidyl)amino]-2-fluoro-phenyl]-4-hydroxy-4-piperidyl]acetic acid

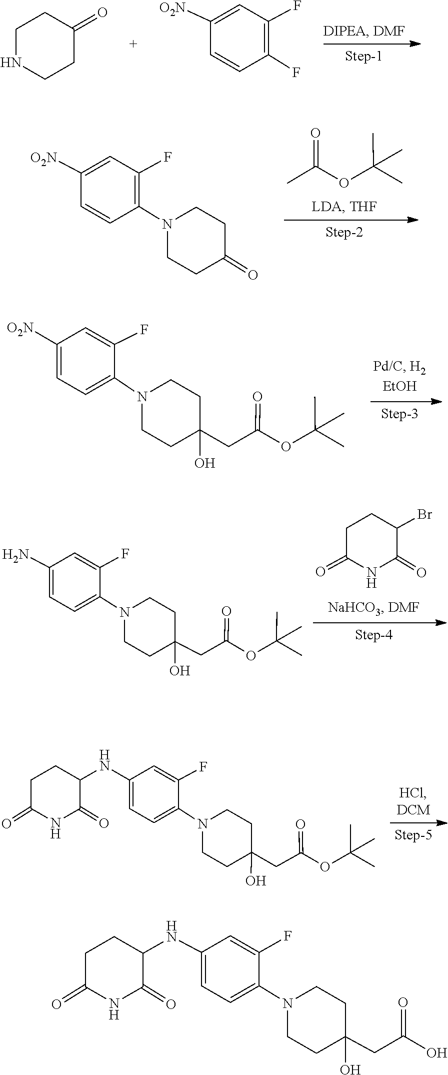

### Step-1:

To a stirred solution of piperidin-4-one HCl salt (20 g, 147.50 mmol) and 1,2-difluoro-4-nitro-benzene (26.99 g, 169.63 mmol, 18.74 mL) in DMSO (200 mL) was added N,N-diisopropylethylamine (19.06 g, 147.50 mmol, 25.69 mL). The reaction was stirred at 80° C. overnight and was monitored by TLC. After 16 hours and complete consumption of the reactant as observed by TLC, ice cold water was added to the reaction mixture and the solid was filtered through Buchner funnel. The solid was dried completely to obtain 1-(2-fluoro-4-nitro-phenyl)piperidin-4-one (28 g, 115.66 mmol, 78.41% yield). LC-MS (ES⁻): m/z 237.1 [M−H]⁻.

### Step-2:

To a stirred solution of tert-butyl acetate (7.31 g, 62.97 mmol, 8.47 mL) in THF was added lithium diisopropylamide (13.49 g, 125.94 mmol) at −78′C. The mixture was allowed to stir for an hour, after which 1-(2-fluoro-4-nitro-phenyl)piperidin-4-one (15 g, 62.97 mmol) was added. The reaction was continued under nitrogen atmosphere for 2 hours. After completion of the reaction, the mixture was quenched with saturated ammonium chloride solution and the product was extracted with ethyl acetate (2×200 mL) and concentrated to provide the crude product. The crude product was purified using flash column chromatography (silica gel, 40% ethyl acetate in pet ether) to afford tert-butyl 2-[1-(2-fluoro-4-nitro-phenyl)-4-hydroxy-4-piperidyl]acetate (17.6 g, 43.71 mmol, 69.41% yield) as a gummy brown liquid. LC-MS (ES⁺): m/z 355.2 [M+H]⁺.

### Step-3:

To the stirred solution of tert-butyl 2-[1-(2-fluoro-4-nitro-phenyl)-4-hydroxy-4-piperidyl]acetate (17.6 g, 49.67 mmol) in ethanol (200 mL) was added Palladium, 10% on carbon, type 487, dry (15 g, 140.95 mmol). The reaction was carried out under hydrogen atmosphere at room temperature for 5 hours. The reaction was monitored by TLC. Upon completion of the reaction, the reaction mixture was concentrated, and the crude product was purified using flash column chromatography (silica gel, 45% ethyl acetate in pet ether) to afford compound tert-butyl 2-[1-(4-amino-2-fluoro-phenyl)-4-hydroxy-4-piperidyl]acetate (13 g, 38.99 mmol, 78.51% yield). LC-MS (ES⁺): m/z 325.2 [M+H]⁺.

### Step-4:

To a stirred solution of tert-butyl 2-[1-(4-amino-2-fluoro-phenyl)-4-hydroxy-4-piperidyl]acetate (13 g, 40.08 mmol) and 3-bromopiperidine-2,6-dione (15.39 g, 80.15 mmol) in DMF (100 mL) was added sodium bicarbonate (6.73 g, 80.15 mmol). The reaction was carried out at 65° C. overnight and was monitored by TLC. After completion of the reaction, the product was extracted by workup with ethyl acetate and water. The extracted organic layer was dried over anhydrous sodium sulfate and concentrated under high vacuum to get the crude, which was purified using flash column chromatography (silica gel, 45% ethyl acetate in pet ether to give tert-butyl 2-[1-[4-[(2,6-dioxo-3-piperidyl)amino]-2-fluoro-phenyl]-4-hydroxy-4-piperidyl]acetate (11.5 g, 65.41% yield). LC-MS (ES⁺): m/z 436.2 [M+H]⁺.

### Step-5:

To the stirred solution of tert-butyl 2-[1-[4-[(2,6-dioxo-3-piperidyl)amino]-2-fluoro-phenyl]-4-hydroxy-4-piperidyl]acetate (411 mg, 943.77 μmol) in DCM (10 mL) was added hydrogen chloride in 1,4-dioxane, 99% (4 M, 4.72 mL) dropwise at 0° C. The reaction mixture stirred at room temperature for 24 hours and monitored by UPLC. After the reaction was complete, the reaction mixture was evaporated to dryness under reduced pressure. The product was redissolved in DCM, and MTBE was added to afford precipitation. Centrifugation to decant the solid. The solvent was removed. The solid was dried under high vacuum to give 2-[1-[4-[(2,6-dioxo-3-piperidyl)amino]-2-fluoro-phenyl]-4-hydroxy-4-piperidyl]acetic acid HCl salt (365 mg, 789.96 μmol, 83.70% yield) as a gray solid. LC-MS (ES⁺): m/z 380.3 [M+H]⁺.

### Synthesis of 2-[1-[2-chloro-4-[(2,6-dioxo-3-piperidyl)amino]phenyl]-4-hydroxy-4-piperidyl]acetic acid

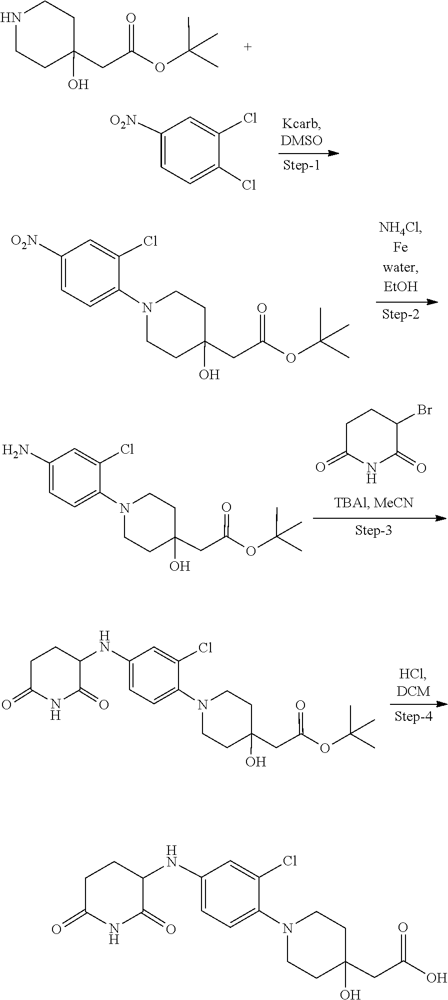

### Step-1:

To a solution of 1,2-dichloro-4-nitro-benzene (5 g, 26.04 mmol) and 1,2-dichloro-4-nitro-benzene (5 g, 26.04 mmol) in DMSO (50 mL) was added potassium carbonate (10.80 g, 78.13 mmol). The mixture was stirred at 110° C. for 1 hour. The reaction was cooled to 20° C. and poured into water (500 mL) and the mixture was extracted with EtOAc (200 mL×3). The combined organic phase was washed with brine (200×2 mL), dried with anhydrous sodium sulfate, filtered and concentrated in vacuo to afford tert-butyl 2-[1-(2-chloro-4-nitro-phenyl)-4-hydroxy-4-piperidyl]acetate (9.4 g, 22.8 mmol, 87.6% yield). ¹H NMR (400 MHz, DMSO-d₆) δ=8.20 (d, J=2.8 Hz, 1H), 8.12 (dd, J=2.8, 8.8 Hz, 1H), 7.28 (d, J=8.8 Hz, 1H), 4.65 (s, 1H), 3.29 (br d, J=12.0 Hz, 2H), 3.19-3.08 (m, 2H), 2.39 (s, 2H), 1.88-1.78 (m, 2H), 1.76-1.67 (m, 2H), 1.41 (s, 9H).

### Step-2:

A mixture of tert-butyl 2-[1-(2-chloro-4-nitro-phenyl)-4-hydroxy-4-piperidyl]acetate (9.4 g, 25.35 mmol) in ethanol (190 mL) and water (38 mL) was added ammonium chloride (4.07 g, 76.05 mmol) and iron powder (4.25 g, 76.05 mmol). The reaction mixture was stirred at 90° C. for 16 hours. After the reaction was complete, the reaction mixture was filtered to remove iron powder, concentrated to remove solvent. It was then poured into water (400 mL) and the mixture was extracted with EtOAc (200 mL×3). The combined organic phase was washed with brine (200 mL×2), dried with anhydrous sodium sulfate, filtered, and concentrated in vacuo to give tert-butyl 2-[1-(4-amino-2-chloro-phenyl)-4-hydroxy-4-piperidyl]acetate (8.64 g, 22.94 mmol, 90% yield). ¹H NMR (400 MHz, DMSO-d₆) δ=6.88 (d, J=8.4 Hz, 1H), 6.61 (d, J=2.4 Hz, 1H), 6.47 (dd, J=2.4, 8.4 Hz, 1H), 4.96 (br s, 2H), 4.43 (s, 1H), 2.89-2.80 (m, 2H), 2.79-2.72 (m, 2H), 2.34 (s, 2H), 1.82-1.72 (m, 2H), 1.68-1.60 (m, 2H), 1.41 (s, 9H).

### Step-3:

To a stirred solution of tert-butyl 2-[1-(4-amino-2-chloro-phenyl)-4-hydroxy-4-piperidyl]acetate (6.4 g, 18.78 mmol) in acetonitrile (100 mL) was added TBAI (13 g, 9.39 mmol), NaHCO₃ (4.41 g, 56.33 mmol). After 5 minutes of stirring, 3-bromopiperidine-2,6-dione (3.61 g, 18.78 mmol) was added at room temperature. After 10 minutes, the temperature of the reaction was raised to 90° C. and the reaction continued for about 72 hours. The reaction mixture was concentrated under reduced pressure to remove solvent. The residue was diluted with water (400 mL) and extracted with EtOAc (150 mL×3). The combined organic layers were washed with brine (20 mL×2), dried over anhydrous sodium sulfate, filtered and concentrated under reduced pressure. The residue was purified by column chromatography (silica gel, petroleum ether/ethyl acetate=1:1) to give tert-butyl 2-[1-[2-chloro-4-[(2,6-dioxo-3-piperidyl)amino]phenyl]-4-hydroxy-4-piperidyl]acetatecarbamate (4.0 g, 8.41 mmol, 44.8% yield) as a blue solid. ¹H NMR (400 MHz, DMSO-d₆) δ=10.78 (s, 1H), 6.95 (d, J=8.8 Hz, 1H), 6.74 (d, J=2.4 Hz, 1H), 6.59 (dd, J=2.4, 8.8 Hz, 1H), 5.83 (d, J=8.0 Hz, 1H), 4.47 (s, 1H), 4.32-4.25 (m, 1H), 2.91-2.83 (m, 2H), 2.81-2.75 (m, 2H), 2.74-2.68 (m, 1H), 2.58 (t, J=4.0 Hz, 1H), 2.35 (s, 2H), 2.11-2.03 (m, 1H), 1.85 (dd, J=4.4, 12.0 Hz, 1H), 1.81-1.73 (m, 2H), 1.68-1.61 (m, 2H), 1.41 (s, 9H). LC-MS (ES⁺): m/z 452.2 [M+H]⁺.

### Step-4:

Into a 25 mL single-neck round-bottom flask containing a well-stirred solution of tert-butyl 2-[1-[2-chloro-4-[(2,6-dioxo-3-piperidyl)amino]phenyl]-4-hydroxy-4-piperidyl]acetate (150 mg, 331.90 μmol) in anhydrous DCM (2 mL) was added 4 M HCl in 1,4 dioxane (331.90 μmol, 3 mL) at ambient temperature under nitrogen atmosphere. The resulting mixture was stirred at room temperature for 16 hours. The reaction mixture was concentrated under reduced pressure to afford 2-[1-[2-chloro-4-[(2,6-dioxo-3-piperidyl)amino]phenyl]-4-hydroxy-4-piperidyl]acetic acid HCl salt (140 mg, 320.61 μmol, 96.60% yield) as an off-white solid. LC-MS (ES⁺): m/z 396.1 [M+H]⁺.

### Synthesis of 2-[4-[4-(2,6-dioxo-3-piperidyl)phenyl]-1-piperidyl]acetic acid

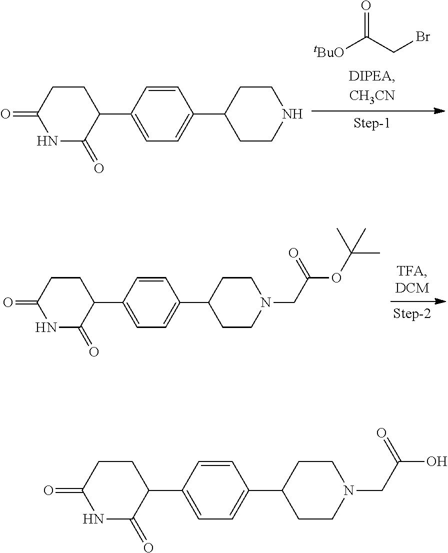 [SMILES: CC(C)(C)OC(=O)CBr ; O=C1CCC(c2ccc(C3CCNCC3)cc2)C(=O)N1 ; CC(C)(C)OC(=O)CN1CCC(c2ccc(C3CCC(=O)NC3=O)cc2)CC1 ; O=C(O)CN1CCC(c2ccc(C3CCC(=O)NC3=O)cc2)CC1]

### Step-1:

A solution of 3-[4-(4-piperidyl)phenyl]piperidine-2,6-dione TFA salt (0.150 g, 388.23 μmol) in acetonitrile (3 mL) was stirred in a sealed tube at room temperature under nitrogen atmosphere. To the reaction mixture was added N,N-diisopropylethylamine (150.53 mg, 1.16 mmol, 202.87 μL), followed by tert-butyl 2-bromoacetate (75.73 mg, 388.23 μmol, 56.94 μL) at the same temperature. The reaction mixture was then stirred at 70° C. for 1 hour. The reaction progress was monitored by TLC and LCMS. After completion of the reaction, the solvent was removed under reduced pressure. The crude product was quenched with water, extracted with ethyl acetate, and washed with brine solution. The combined organic layers were concentrated under reduced pressure to afford tert-butyl 2-[4-[4-(2,6-dioxo-3-piperidyl)phenyl]-1-piperidyl]acetate (0.150 g, 319.69 μmol, 82.34% yield) as a light yellow color solid. LC-MS (ES⁺): m/z 387.50 [M+H]⁺.

### Step-2:

To a stirred solution of tert-butyl 2-[4-[4-(2,6-dioxo-3-piperidyl)phenyl]-1-piperidyl]acetate (0.2 g, 517.49 μmol) in DCM (5 mL) was added 2,2,2-trifluoroacetic acid (885.08 mg, 7.76 mmol, 598.03 μL) at 0° C. under N₂ atmosphere. The reaction mixture was stirred at room temperature for 12 hours. The progress of the reaction was monitored by TLC. Upon completion, the reaction mixture was concentrated in vacuo and the residue was washed with diethyl ether (5 mL) to afford 2-[4-[4-(2,6-dioxo-3-piperidyl)phenyl]-1-piperidyl]acetic acid TFA salt (0.14 g, 226.29 μmol, 43.73% yield) a black gummy. LC-MS (ES⁺): m/z 331.46 [M+H]⁺.

### Synthesis of 2-[4-[4-(3-fluoro-2,6-dioxo-3-piperidyl)phenyl]-1-piperidyl]acetic acid

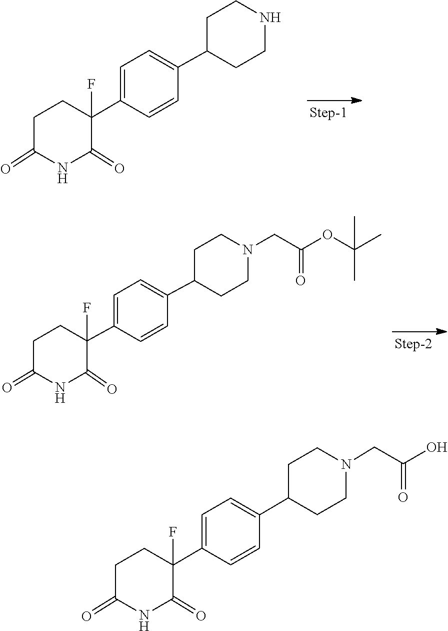 [SMILES: O=C1CCC(F)(c2ccc(C3CCNCC3)cc2)C(=O)N1 ; CC(C)(C)OC(=O)CN1CCC(c2ccc(C3(F)CCC(=O)NC3=O)cc2)CC1 ; O=C(O)CN1CCC(c2ccc(C3(F)CCC(=O)NC3=O)cc2)CC1]

Procedures are similar to those of 2-[4-[4-(2,6-dioxo-3-piperidyl)phenyl]-1-piperidyl]acetic acid, except the synthesis started with 3-fluoro-3-[4-(4-piperidyl)phenyl]piperidine-2,6-dione.

### Step-1:

Compound tert-butyl 2-[4-[4-(3-fluoro-2,6-dioxo-3-piperidyl)phenyl]-1-piperidyl]acetate (0.070 g, 162.68 μmol, 29.90% yield) was obtained as a colorless semi solid. LC-MS (ES⁺): m/z 405.31 [M+H]⁺.

### Step-2:

Compound 2-[4-[4-(3-fluoro-2,6-dioxo-3-piperidyl)phenyl]-1-piperidyl]acetic acid TFA salt (0.060 g, 90.83 μmol, 52.48% yield) was obtained as colorless semi solid. LC-MS (ES⁺): m/z 349.65 [M+H]⁺.

### Synthesis of 2-(4-(5-((2,6-dioxopiperidin-3-yl)amino)pyridin-2-yl)piperidin-1-yl)acetic acid

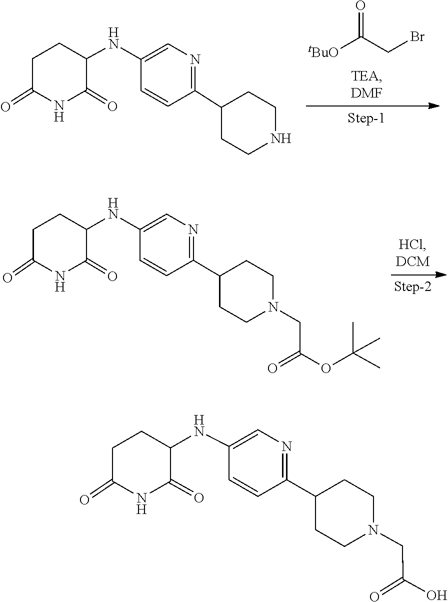 [SMILES: O=C1CCC(Nc2ccc(C3CCNCC3)nc2)C(=O)N1 ; CC(C)(C)OC(=O)CBr ; CC(C)(C)OC(=O)CN1CCC(c2ccc(NC3CCC(=O)NC3=O)cn2)CC1 ; O=C(O)CN1CCC(c2ccc(NC3CCC(=O)NC3=O)cn2)CC1]

### Step-1:

To a solution of 3-[[6-(4-piperidyl)-3-pyridyl]amino]piperidine-2,6-dione (130 mg, 450.85 μmol) and tert-butyl 2-bromoacetate (105.53 mg, 541.02 μmol, 79.34 μL) in DMF (5 mL) was added TEA (364.97 mg, 3.61 mmol, 502.72 μL). The mixture was stirred at 25° C. for 16 hours. After LC-MS showed the complete consumption of the reactant, the reaction mixture was concentrated under reduced pressure to remove DMF and the residue was purified by reverse phase prep-HPLC (ACSWH-GX-O/Phenomenex Luna C18 75×30 mm×3 um/water(0.1% TFA)-ACN/Begin B:2-End B:32/Gradient Time(min): 7). Compound tert-butyl 2-[4-[5-[(2,6-dioxo-3-piperidyl)amino]-2-pyridyl]-1-piperidyl]acetate (90 mg, 223.61 μmol, 49.60% yield) was obtained as a white solid. LC-MS (ES⁺): m/z 403.2 [M+H]⁺.

### Step-2:

To a solution of tert-butyl 2-(4-(5-((2,6-dioxopiperidin-3-yl)amino)pyridin-2-yl)piperidin-1-yl)acetate (90 mg, 223.61 μmol) in DCM (1 mL) was added HCl (12 M, 186.34 μL) and the mixture was stirred at 25° C. for 5 hours. After complete consumption of the reactant as confirmed by LC-MS, the reaction mixture was concentrated under reduced pressure to remove DCM. Compound 2-(4-(5-((2,6-dioxopiperidin-3-yl)amino)pyridin-2-yl)piperidin-1-yl)acetic acid HCl salt (60 mg, 156.72 μmol, 70.09% yield) was obtained as a white solid. LC-MS (ES⁺): m/z 347.15 [M+H]⁺.

### Synthesis of 2-[4-[4-(2,6-dioxo-3-piperidyl)-2-fluoro-phenyl]-1-piperidyl]acetic acid

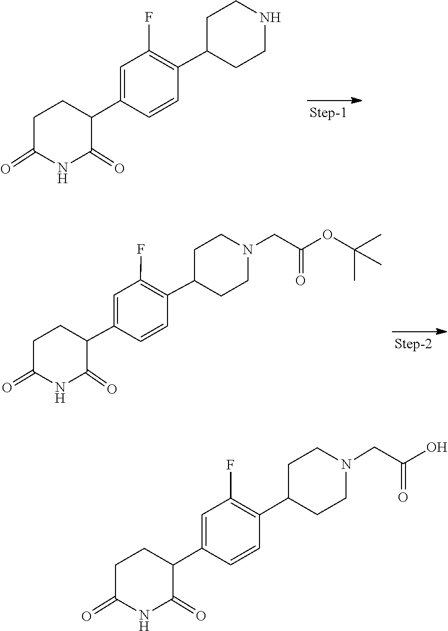 [SMILES: O=C1CCC(c2ccc(C3CCNCC3)c(F)c2)C(=O)N1 ; CC(C)(C)OC(=O)CN1CCC(c2ccc(C3CCC(=O)NC3=O)cc2F)CC1 ; O=C(O)CN1CCC(c2ccc(C3CCC(=O)NC3=O)cc2F)CC1]

Procedures are identical to those of 2-(4-(5-((2,6-dioxopiperidin-3-yl)amino)pyridin-2-yl)piperidin-1-yl)acetic acid, except the synthesis started with 3-(3-fluoro-4-(piperidin-4-yl)phenyl)piperidine-2,6-dione.

### Step-1:

Compound tert-butyl 2-(4-(4-(2,6-dioxopiperidin-3-yl)-2-fluorophenyl)piperidin-1-yl)acetate (147 mg, 348.90 μmol, 33.77% yield) was obtained as a white solid. LC-MS (ES⁺): m/z 405.2 [M+H]⁺.

### Step-2:

Compound 2-[4-[4-(2,6-dioxo-3-piperidyl)-2-fluoro-phenyl]-1-piperidyl]acetic acid HCl salt (130 mg, 337.81 μmol, 97.60% yield) was obtained as a white solid. LC-MS (ES⁺): m/z 349.0 [M+H]⁺.

### Synthesis of 2-(4-(4-(2,6-dioxopiperidin-3-yl)-2,5-difluorophenyl)piperidin-1-yl)acetic acid

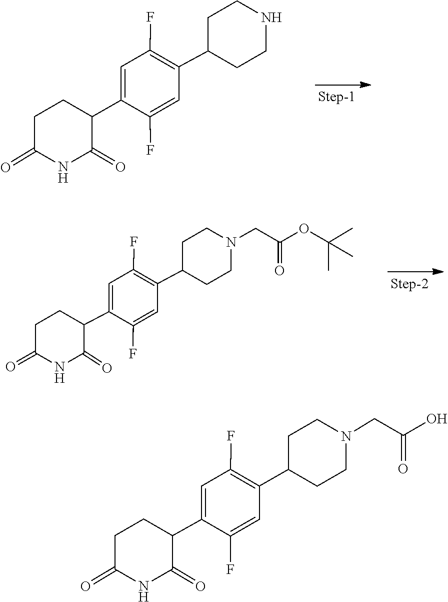 [SMILES: O=C1CCC(c2cc(F)c(C3CCNCC3)cc2F)C(=O)N1 ; CC(C)(C)OC(=O)CN1CCC(c2cc(F)c(C3CCC(=O)NC3=O)cc2F)CC1 ; O=C(O)CN1CCC(c2cc(F)c(C3CCC(=O)NC3=O)cc2F)CC1]

Procedures are identical to those of 2-(4-(5-((2,6-dioxopiperidin-3-yl)amino)pyridin-2-yl)piperidin-1-yl)acetic acid, except the synthesis started with 3-[2,5-difluoro-4-(4-piperidyl)phenyl]piperidine-2,6-dione.

### Step-1:

Compound tert-butyl 2-(4-(4-(2,6-dioxopiperidin-3-yl)-2,5-difluorophenyl)piperidin-1-yl)acetate (240 mg, 568.10 μmol, 70.06% yield) was obtained as a white solid. LC-MS (ES⁺): m/z 423.2[M+H]⁺.

### Step-2:

Compound 2-(4-(4-(2,6-dioxopiperidin-3-yl)-2,5-difluorophenyl)piperidin-1-yl)acetic acid (200 mg, 545.91 μmol, 96.10% yield) was obtained as an off-white solid. The crude product was checked by TLC and used directly in the next step without purification.

### Synthesis of 2-[1-[5-[(2,6-dioxo-3-piperidyl)amino]-3-fluoro-2-pyridyl]-4-piperidyl]acetic acid

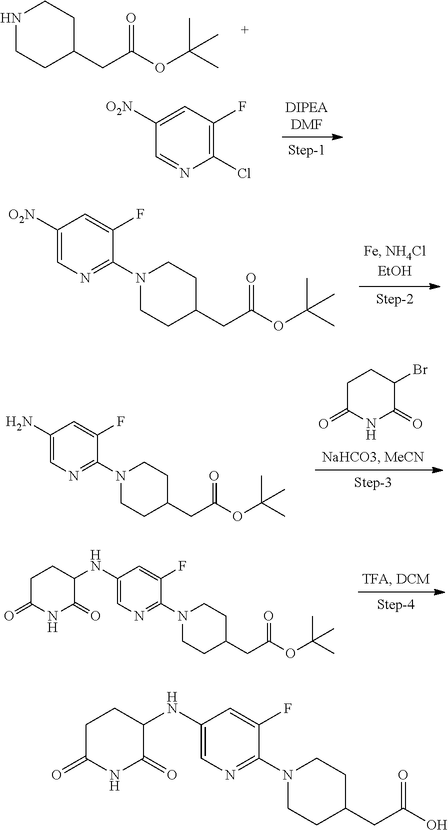

### Step-1:

A 100 mL single neck round bottom flask containing a well stirred solution of tert-butyl 2-(4-piperidyl)acetate (2 g, 10.04 mmol) in DMF (30 mL) were added DIPEA (3.89 g, 30.11 mmol, 5.24 mL) followed by 2-chloro-3-fluoro-5-nitro-pyridine (1.77 g, 10.04 mmol). The reaction mixture was stirred a 90° C. for 12 hours and monitored by TLC. After completion of the reaction, the reaction mixture was cooled to room temperature and quenched with cold water (300 mL). The aqueous layer was extracted with EtOAc (3×100 mL) and the combined organic layer was washed with water (2×100 mL), brine solution, dried over anhydrous sodium sulfate and concentrated under reduced pressure to afford the tert-butyl 2-[1-(3-fluoro-5-nitro-2-pyridyl)-4-piperidyl]acetate (2.5 g, 6.58 mmol, 65.52% yield) as a dark brown solid. LC-MS (ES⁺): m/z 340.2 [M+H]⁺.

### Step-2:

Into a 250 mL single neck round bottom flask containing a well stirred solution of tert-butyl 2-[1-(3-fluoro-5-nitro-2-pyridyl)-4-piperidyl]acetate (1 g, 2.45 mmol) in ethanol (25 mL) and water (8 mL) were added a solution of ammonium chloride (656.50 mg, 12.27 mmol, 429.09 μL) in water (3 mL). This was followed by the portionwise addition of iron powder (685.39 mg, 12.27 mmol) at room temperature. The reaction mixture was heated to 75° C. and stirred for 4 hours. After 58% of the product was detected by LC-MS, the reaction mixture was cooled to room temperature, filtered through celite bed and washed with EtOAc (50 mL). The reaction mixture was concentrated under reduced pressure, diluted with water (40 mL), and extracted with EtOAc (3×40 mL). The combined organic layers were washed with water (2×40 mL), brine solution (40 mL), dried over anhydrous sodium sulfate and concentrated under reduced pressure. The crude product was purified by column chromatography (silica gel, 65-70% EtOAC in pet ether) to afford tert-butyl 2-[1-(5-amino-3-fluoro-2-pyridyl)-4-piperidyl]acetate (570 mg, 1.76 mmol, 71.68% yield) as a brown gum. LC-MS (ES⁺): m/z 310.2 [M+H]⁺.

### Step-3:

Into a 50 single neck round bottom flask containing a well stirred solution of tert-butyl 2-[1-(5-amino-3-fluoro-2-pyridyl)-4-piperidyl]acetate (780 mg, 2.43 mmol) in acetonitrile (15 mL) were added sodium bicarbonate (1.02 g, 12.13 mmol) followed by of 3-bromopiperidine-2,6-dione (931.40 mg, 4.85 mmol) and the resultant reaction mixture was stirred at 80° C. for 16 hours. The reaction was monitored by LC-MS. After about 60% conversion, 3-bromopiperidine-2,6-dione (931.40 mg, 4.85 mmol) was added to the reaction mixture and it was stirred for an additional 16 hours. After about 65% conversion, the reaction mixture was cooled to room temperature and concentrated under reduced pressure. The residue was purified by flash column chromatography (silica gel, 65-80% EtOAc in pet-ether) to afford the crude product, which was further purified by reverse phase column chromatography (30 g of HP-C18 column using a gradient of 0.1% NH₄OAc in CAN with the desired product eluting at 49-53% 0.1% NH₄OAc in ACN (10 mL/min flow rate) to afford tert-butyl 2-[1-[5-[(2,6-dioxo-3-piperidyl)amino]-3-fluoro-2-pyridyl]-4-piperidyl]acetate (400 mg, 948.45 μmol, 39.11% yield) as a beige solid. LC-MS (ES⁺): m/z 421.2 [M+H]⁺.

### Step-4:

Into a 25 mL single neck round bottom flask containing a well stirred solution of tert-butyl 2-[1-[5-[(2,6-dioxo-3-piperidyl)amino]-3-fluoro-2-pyridyl]-4-piperidyl]acetate (130 mg, 242.39 μmol) in DCM (3 mL) was added TFA (1.48 g, 12.98 mmol, 1 mL) dropwise at 0° C. The reaction mixture was stirred at room temperature for 1 hour and monitored by LC-MS. After completion of the reaction, the volatiles were distilled off under reduced pressure to get a brown residue, which was triturated with MTBE (10 mL) to afford crude 2-[1-[5-[(2,6-dioxo-3-piperidyl)amino]-3-fluoro-2-pyridyl]-4-piperidyl]acetic acid TFA salt (102 mg, 183.36 μmol, 75.65% yield) as a brown gum. LC-MS (ES⁺): m/z 365.2 (M+H)+.

### Synthesis of 3-((5-(piperidin-4-yl)pyridin-2-yl)amino)piperidine-2,6-dione

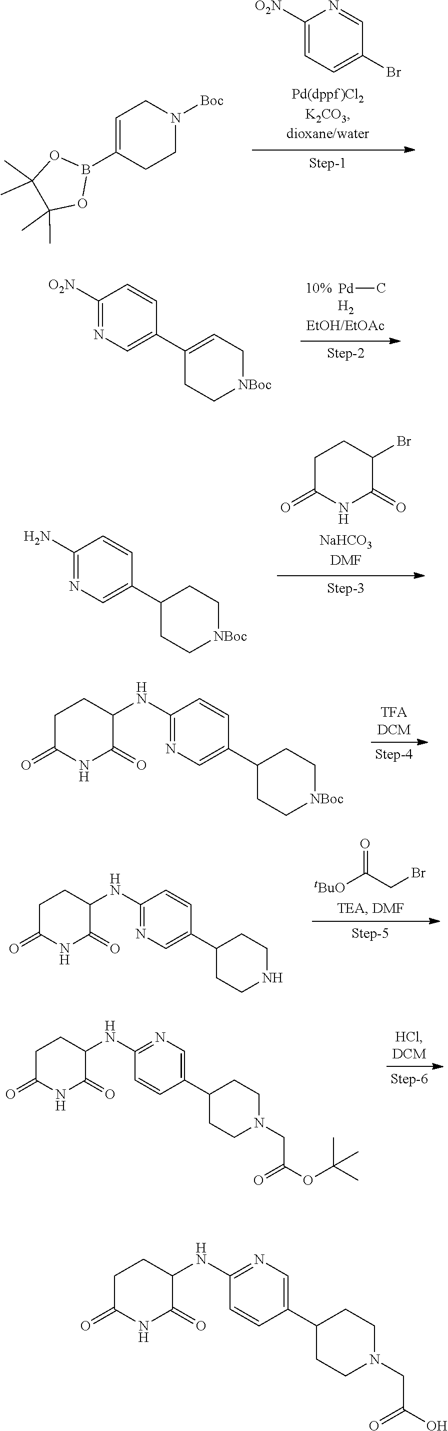

### Step-1:

A solution of 5-bromo-2-nitro-pyridine (15 g, 73.89 mmol) in dioxane (150 mL) were added tert-butyl 4-(4,4,5,5-tetramethyl-1,3,2-dioxaborolan-2-yl)-3,6-dihydro-2H-pyridine-1-carboxylate (25.13 g, 81.28 mmol), potassium carbonate, anhydrous, 99% (30.64 g, 221.68 mmol) in water (30 mL). The mixture was purged with nitrogen gas for 20 minutes before cyclopentyl(diphenyl)phosphane;dichloropalladium;iron (2.70 g, 3.69 mmol) was added and the reaction refluxed at 80° C. for 4 hours. The reaction progress was monitored by TLC and LC-MS. After completion, the reaction was diluted with cold water and extracted with ethyl acetate. The organic layer was washed with brine solution and concentrated to dryness. The resulting crude product was purified by column chromatography(silica gel 100-200 mesh, 0-20% ethyl acetate in pet ether) to afford tert-butyl 4-(6-nitro-3-pyridyl)-3,6-dihydro-2H-pyridine-1-carboxylate (18 g, 57.18 mmol, 77.39% yield) as an off-white solid. LC-MS (ES⁺): m/z 306.42[M+H]⁺.

### Step-2:

To a stirred solution of tert-butyl 4-(6-nitro-3-pyridyl)-3,6-dihydro-2H-pyridine-1-carboxylate (5 g, 16.38 mmol) in ethyl acetate (50 mL) was added palladium, 10% on carbon, type 487, dry (4.36 g, 40.94 mmol). The reaction was stirred under hydrogen gas for 16 hours. The reaction progress was monitored by TLC and LC-MS. After completion, the reaction was filtered through celite bed and washed with ethyl acetate. The filtrate was concentrated in vacuo to afford tert-butyl 4-(6-amino-3-pyridyl)piperidine-1-carboxylate (4.4 g, 15.45 mmol, 94.35% yield) as a solid. LC-MS (ES⁺): m/z 278.46 [M+H]⁺.

### Step-3:

To a stirred solution of tert-butyl 4-(6-amino-3-pyridyl)piperidine-1-carboxylate (2 g, 7.21 mmol) in DMF (20 mL) was added sodium bicarbonate (6.06 g, 72.11 mmol) followed by 3-bromopiperidine-2,6-dione (13.85 g, 72.11 mmol) under argon atmosphere in a sealed tube. The reaction mixture was stirred at 80° C. for 16 hours and the reaction progress was monitored by TLC. The reaction mixture was poured into ice cold water and stirred for 30 minutes. The solid product was separated by filtration and washed with water and pet ether. The product in the filtrate was extracted with ethyl acetate. The solid product was then dissolved in dichloromethane/methanol (5/1) and combined with the extracted product in ethyl acetate. It was dried over sodium sulfate, and evaporated to dryness to obtain the crude product, which was purified by column chromatography (silica gel 230-400 mesh, 0-100% ethyl acetate in pet ether) to afford tert-butyl 4-[6-[(2,6-dioxo-3-piperidyl)amino]-3-pyridyl]piperidine-1-carboxylate (2.8 g, 4.61 mmol, 63.97% yield) as a light yellow solid. LC-MS (ES⁺): m/z 389.25 [M+H]⁺.

### Step-4:

To a solution of tert-butyl 4-[6-[(2,6-dioxo-3-piperidyl)amino]-3-pyridyl]piperidine-1-carboxylate (1.1 g, 2.83 mmol) in dichloromethane (10 mL) was added trifluoroacetic acid (322.88 mg, 2.83 mmol, 218.16 μL) at 0° C. and the reaction was stirred at room temperature for 1 hour. The reaction mixture was then concentrated in vacuo to obtain the crude product, which was triturated with diethyl ether (50 mL) to afford 3-[[5-(4-piperidyl)-2-pyridyl]amino]piperidine-2,6-dione TFA salt (1.1 g, 2.05 mmol, 72.41% yield) as an off-white solid. LC-MS (ES⁺): m/z 289.47 [M+H]⁺.

### Step-5:

To a solution of 3-((5-(piperidin-4-yl)pyridin-2-yl)amino)piperidine-2,6-dione (70 mg, 242.77 μmol) and tert-butyl 2-bromoacetate (52.09 mg, 267.04 μmol, 39.16 μL) in DMF (1 mL) was added TEA (196.52 mg, 1.94 mmol, 270.69 μL). The mixture was stirred at 25° C. for 2 hours. After complete consumption of the reactant as shown by LC-MS, the mixture was diluted with water (25 mL) and extracted with ethyl acetate (20 mL×3). The combined organic layers were washed with brine (10 mL×2), dried over anhydrous sodium sulfate, filtered, and concentrated under reduced pressure. The crude product tert-butyl 2-(4-(6-((2,6-dioxopiperidin-3-yl)amino)pyridin-3-yl)piperidin-1-yl)acetate (107 mg, 265.85 μmol, 109.51% yield) was used in the next step without further purification. LC-MS (ES⁺): m/z 403.3 [M+H]⁺.

### Step-6:

To a solution of tert-butyl 2-[4-[6-[(2,6-dioxo-3-piperidyl)amino]-3-pyridyl]-1-piperidyl]acetate (107 mg, 265.85 μmol) in DCM (1 mL) was added HCl (12 M, 22.15 μL). The mixture was stirred at 25° C. for 1 hour. After complete consumption of the reactant as shown by LC-MS, the reaction mixture was concentrated in vacuo, and the crude product 2-(4-(6-((2,6-dioxopiperidin-3-yl)amino)pyridin-3-yl)piperidin-1-yl)acetic acid HCl salt (108 mg, 282.10 μmol, 106.11% yield) was used in the next step without further purification. LC-MS (ES⁺): m/z 347.15 [M+H]⁺.

### Synthesis of 2-(4-(2-((2,6-dioxopiperidin-3-yl)amino)pyrimidin-5-yl)piperidin-1-yl)acetic acid

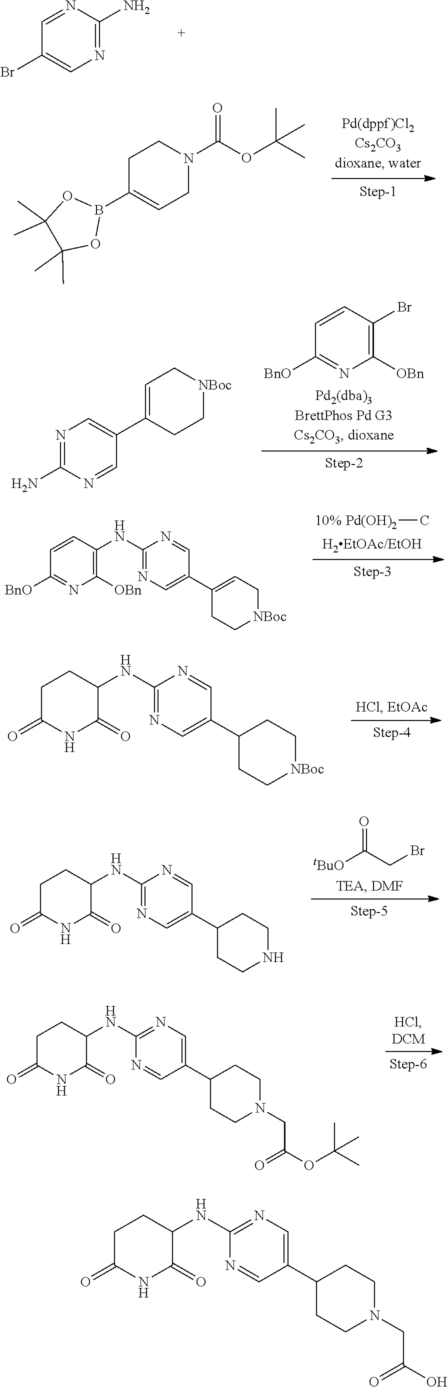

### Step-1:

To the solution of tert-butyl 4-(4,4,5,5-tetramethyl-1,3,2-dioxaborolan-2-yl)-3,6-dihydro-2H-pyridine-1-carboxylate (8.53 g, 27.59 mmol) and 5-bromopyrimidin-2-amine (4 g, 22.99 mmol) in dioxane (40 mL) and water (4 mL) was added cesium carbonate (14.98 g, 45.98 mmol) and cyclopentyl(diphenyl)phosphane;dichloromethane;dichloropalladium;iron (1.88 g, 2.30 mmol), and the reaction mixture was stirred at 120° C. for 12 hours. After completion of the reaction as confirmed by LC-MS, the mixture was filtered and concentrated to give a residue, which was purified by flash column chromatography (silica gel, pet ether/ethyl acetate=3/1-1/1). The desired product tert-butyl 4-(2-aminopyrimidin-5-yl)-3,6-dihydro-2H-pyridine-1-carboxylate (5.27 g, 19.07 mmol, 82.96% yield) was obtained as yellow solid. LC-MS (ES⁺): m/z 277.2 [M+H]⁺.

### Step-2:

To the mixture of tert-butyl 4-(2-aminopyrimidin-5-yl)-3,6-dihydro-2H-pyridine-1-carboxylate (110 mg, 398.07 μmol) and 2,6-dibenzyloxy-3-bromo-pyridine (176.86 mg, 477.68 μmol) in dioxane (5 mL) was added (1E,4E)-1,5-diphenylpenta-1,4-dien-3-one; palladium (36.45 mg, 39.81 μmol), dicyclohexyl(2,4,6-triisopropyl-3,6-dimethoxy-[1,1-biphenyl]-2-yl)phosphine (42.73 mg, 79.61 μmol) and cesium carbonate (389.10 mg, 1.19 mmol), and the reaction was stirred at 100° C. for 12 hours. After completion of the reaction as confirmed by LC-MS, the mixture was purified by flash column chromatography (silica gel, pet ether/ethyl acetate=5/1). The desired product tert-butyl 4-[2-[(2,6-dibenzyloxy-3-pyridyl)amino]pyrimidin-5-yl]-3,6-dihydro-2H-pyridine-1-carboxylate (181 mg, 260.14 μmol, 65.35% yield) was obtained as a yellow oil. LC-MS (ES⁺): m/z 566.2 [M+H]⁺.

### Step-3:

To the solution of tert-butyl 4-[2-[(2,6-dibenzyloxy-3-pyridyl)amino]pyrimidin-5-yl]piperidine-1-carboxylate (1 g, 1.76 mmol) in ethyl acetate (10 mL) was added palladium hydroxide on carbon, 20 wt. % (247.39 mg, 1.76 mmol). The mixture was purged with H₂ three times, and the stirred under H₂ atmosphere at 45° C. for 3 hours. After consumption of the reactant as shown by LC-MS, the mixture was filtered and concentrated to give a residue, which was purified by prep-TLC. (PE/EA=1/2-0/1). The desired product tert-butyl 4-[2-[(2,6-dioxo-3-piperidyl)amino]pyrimidin-5-yl]piperidine-1-carboxylate (315 mg, 680.23 μmol, 38.62% yield) was obtained as brown solid. LC-MS (ES⁺): m/z 389.9 [M+H]⁺.

### Step-4:

To the solution of tert-butyl 4-[2-[(2,6-dioxo-3-piperidyl)amino]pyrimidin-5-yl]piperidine-1-carboxylate (100 mg, 256.77 μmol) in ethyl acetate (5 mL) was added hydrogen chloride solution 1.0 M in ethyl acetate (18.72 mg, 513.55 μmol, 23.41 μL) at 0° C. The reaction was stirred at 25° C. for 2 hours. After consumption of the reactant as shown by LC-MS, the mixture was concentrated to give the crude product 3-[[5-(4-piperidyl)pyrimidin-2-yl]amino]piperidine-2,6-dione HCl salt (43.2 mg, 132.60 μmol, 51.64% yield) as a brown solid. LC-MS (ES⁺): m/z 290.1 [M+H]⁺.

### Step-5:

To a solution of 3-[[5-(4-piperidyl)pyrimidin-2-yl]amino]piperidine-2,6-dione (80 mg, 276.50 μmol) and tert-butyl 2-bromoacetate (59.33 mg, 304.15 μmol, 44.61 μL) in DMF (2 mL) was added TEA (223.83 mg, 2.21 mmol, 308.31 μL). The mixture was stirred at 25° C. for 2 hours. After completion of the reaction as confirmed by LC-MS, the reaction mixture was diluted with water (25 mL) and extracted with ethyl acetate (20 mL×3). The combined organic layers were washed with brine (10 mL×2), dried over anhydrous sodium sulfate, filtered, and concentrated under reduced pressure. The crude product tert-butyl 2-(4-(2-((2,6-dioxopiperidin-3-yl)amino)pyrimidin-5-yl)piperidin-1-yl)acetate (80 mg, 198.28 μmol, 71.71% yield) was used in the next step without further purification. LC-MS (ES⁺): m/z 404.3 [M+H]⁺.

### Step-6:

To a solution of tert-butyl 2-[4-[2-[(2,6-dioxo-3-piperidyl)amino]pyrimidin-5-yl]-1-piperidyl]acetate (80 mg, 198.28 μmol) in DCM (1 mL) was added HCl (12 M, 165.23 μL) and the mixture was stirred at 25° C. for 1 hour. After complete consumption of the reactant as shown by LC-MS, the reaction mixture was concentrated in vacuo to give the crude product 2-(4-(2-((2,6-dioxopiperidin-3-yl)amino)pyrimidin-5-yl)piperidin-1-yl)acetic acid HCl salt (95 mg, 247.51 μmol, 124.83% yield), which was used in the next step without further purification. LC-MS (ES⁺): m/z 348.15 [M+H]⁺.

### Synthesis of 2-(4-(3-((2,6-dioxopiperidin-3-yl)amino)-1H-pyrazol-1-yl)piperidin-1-yl)acetic acid

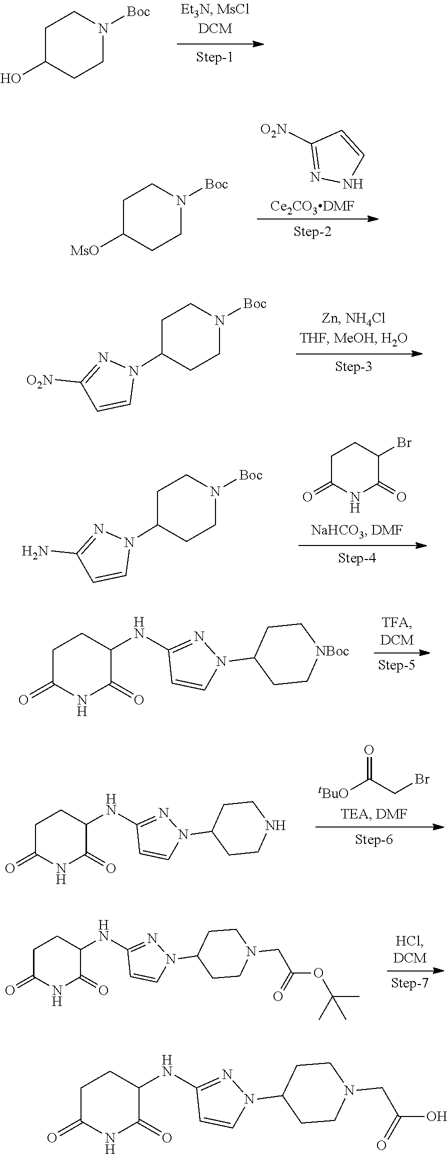

### Step-1:

To a solution of tert-butyl 4-hydroxypiperidine-1-carboxylate (30 g, 149.06 mmol) in DCM (300 mL) was added triethyl amine (150.83 g, 1.49 mol, 207.76 mL) and stirred for 5 minutes. Mesyl chloride (25.61 g, 223.59 mmol, 17.31 mL) was added to reaction mixture at 0° C. and the resulting mixture was stirred at 27° C. for 16 hours. The reaction mixture was quenched with water and extracted with DCM (100 mL×3). The organic layer was washed with water (100 mL) and brine solution (100 mL). The combined organic layers were dried over anhydrous sodium sulfate, filtered, and concentrated under reduced pressure. The crude product was purified by column chromatography (silica gel 230-400 mesh, 0-100% ethyl acetate in pet ether) to afford tert-butyl 4-methylsulfonyloxy piperidine-1-carboxylate (40 g, 136.03 mmol, 91.26% yield, 95% purity) tert-butyl 4-methylsulfonyloxypiperidine-1-carboxylate (40 g, 136.03 mmol, 91.26% yield). ¹H NMR (400 MHz, DMSO-d₆) δ 4.84-4.79 (m, 1H), 3.63-3.57 (m, 2H), 3.17-2.51 (m, 5H), 1.93-1.88 (m, 2H), 1.65-1.56 (m, 2H), 1.40 (s, 9H).

### Step-2:

To a solution of 3-nitro-1H-pyrazole (10 g, 88.44 mmol) and tert-butyl 4-methyl sulfonyloxypiperidine-1-carboxylate (37.06 g, 132.66 mmol) in DMF (200 mL) was added cesium carbonate (86.44 g, 265.31 mmol) and the reaction was stirred for 16 hours at 65° C. Then, the reaction mixture was quenched by water and extracted by ethyl acetate. The organic layer was concentrated under reduced pressure and the crude mixture was purified by column chromatography (30%-40% ethyl acetate in pet ether) to afford tert-butyl 4-(3-nitropyrazol-1-yl)piperidine-1-carboxylate (4 g, 11.88 mmol, 13.43% yield) as a white semi liquid. LC-MS (ES⁺): m/z 241.2 [[M-C(CH₃)₃]+H]+H]⁺.

### Step-3:

To a solution of tert-butyl 4-(3-nitropyrazol-1-yl)piperidine-1-carboxylate (4 g, 13.50 mmol) in THE (20 mL) and methanol (20 mL) was added ammonia;hydrochloride (14.44 g, 269.98 mmol) in water (5 mL), followed by the addition of a suspension of zinc (8.83 g, 134.99 mmol). The reaction mixture was stirred at room temperature for 16 hours. Upon the completion of the reaction, the mixture was passed through celite bed and the filtrate was diluted with water (50 ml) and extracted by ethyl acetate (250 ml). The organic layer was separated and dried over anhydrous Na₂SO₄. The organic layer was evaporated under vacuum to get the crude compound, which was purified by column chromatography (Devisil silica, 0-100% ethyl acetate in hexane) to give tert-butyl 4-(3-aminopyrazol-1-yl)piperidine-1-carboxylate (2.5 g, 6.57 mmol, 48.68% yield) as a brown solid. LC-MS (ES⁺): m/z 211.2 [[M-C(CH₃)₃]+H]+H]⁺.

### Step-4:

To a solution of tert-butyl 4-(3-aminopyrazol-1-yl)piperidine-1-carboxylate (2.0 g, 7.51 mmol) and 3-bromopiperidine-2,6-dione (4.33 g, 22.53 mmol) in DMF (10 mL) was added sodium bicarbonate (6.31 g, 75.09 mmol) in a sealed tube. The reaction mixture was stirred at 75° C. for 16 hours. Upon completion of the reaction, the mixture was poured in ice cooled water and extracted using ethyl acetate. The organic layer was washed with cooled brine solution to get the crude product. It was purified by reverse phase chromatography over celite using 10% formic acid in water to get tert-butyl 4-[3-[(2,6-dioxo-3-piperidyl)amino]pyrazol-1-yl]piperidine-1-carboxylate (1.1 g, 2.84 mmol, 37.83% yield) as a light ash color solid. LC-MS (ES⁺): m/z 378.3 [M+H]⁺.

### Step-5:

To a solution of tert-butyl 4-[3-[(2,6-dioxo-3-piperidyl)amino]pyrazol-1-yl]piperidine-1-carboxylate (0.900 g, 2.38 mmol) in DCM (10 mL) was added 2,2,2-trifluoroacetic acid (271.89 mg, 2.38 mmol, 183.71 μL) at 0° C. and the reaction mixture was stirred at room temperature for 2 hours. The reaction mixture was concentrated in vacuo and triturated with diethyl ether (100 mL) to afford 3-[[1-(4-piperidyl)pyrazol-3-yl]amino]piperidine-2,6-dione (0.900 g, 1.61 mmol, 67.51% yield) as a black solid. LC-MS (ES⁺): m/z 278.5 [M+H]⁺.

### Step-6:

To a solution of 3-((1-(piperidin-4-yl)-1H-pyrazol-3-yl)amino)piperidine-2,6-dione (180 mg, 649.07 μmol) and tert-butyl 2-bromoacetate (139.26 mg, 713.97 μmol, 104.71 μL) in DMF (2 mL) was added TEA (525.43 mg, 5.19 mmol, 723.73 μL). The mixture was stirred at 25° C. for 2 hours. After complete consumption of the reactant as shown by LC-MS, the reaction was diluted with water (15 mL) and extracted with ethyl acetate (10 mL×3). The combined organic layers were washed with brine (5 mL×2), dried over anhydrous sodium sulfate, filtered, and concentrated under reduced pressure. Compound tert-butyl 2-(4-(3-((2,6-dioxopiperidin-3-yl)amino)-1H-pyrazol-1-yl)piperidin-1-yl)acetate (120 mg, 288.95 μmol, 44.52% yield) was used in the next step without further purification. LC-MS (ES⁺): m/z 392.2 [M+H]⁺.

### Step-7:

To a solution of tert-butyl 2-[4-[3-[(2,6-dioxo-3-piperidyl)amino]pyrazol-1-yl]-1-piperidyl]acetate (120 mg, 306.54 μmol) in DCM (1 mL) was added HCl (12 M, 255.45 μL). The mixture was stirred at 25° C. for 1 hour. After complete consumption of the reactant as confirmed by LC-MS, the reaction mixture was concentrated in vacuo. Compound 2-(4-(3-((2,6-dioxopiperidin-3-yl)amino)-1H-pyrazol-1-yl)piperidin-1-yl)acetic acid HCl salt (140 mg, 376.53 μmol, 122.83% yield) was used in the next step without further purification. LC-MS (ES⁺): m/z 336.15 [M+H]⁺.

### Synthesis of 2-[2-[4-(2,6-dioxo-3-piperidyl)phenyl]-2-azaspiro[3.3]heptan-6-yl]acetic acid

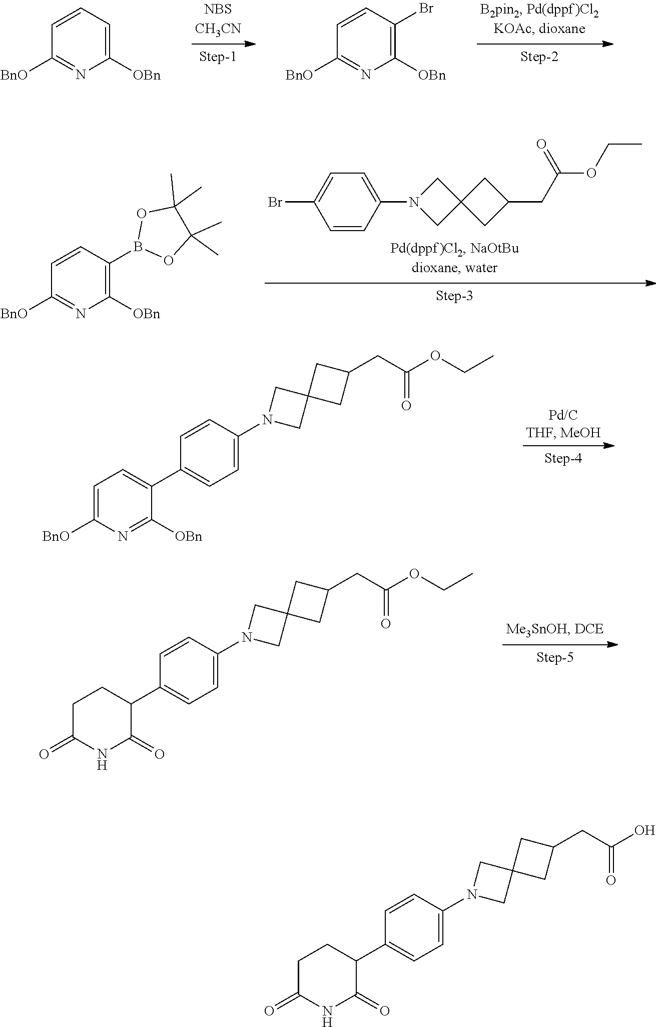

### Step-1:

To a stirred solution of 2,6-dibenzyloxypyridine (57 g, 144.78 mmol) in acetonitrile (500 mL) was added N-bromosuccinimide (25.77 g, 144.78 mmol) at room temperature. The resulting reaction mixture was stirred at 80° C. for 12 hours. The progress of the reaction was monitored by LC-MS and TLC. After consumption of the starting material as indicated by TLC, the reaction mixture was concentrated under reduced pressure to give the crude product, which was partitioned between EtOAc (2×250 mL) and water (100 mL). The organic layer was washed with brine solution (100 mL) and dried over anhydrous sodium sulfate and filtered. The filtrate was concentrated under reduced pressure to afford 2,6-dibenzyloxy-3-bromo-pyridine (60 g, 90.75 mmol, 62.68% yield) as an off-white solid. LC-MS (ES⁺): m/z 292.2 [M-Br+H]⁺.

### Step-2:

To a stirred solution of 2,6-dibenzyloxy-3-bromo-pyridine (35 g, 94.53 mmol) and 4,4,5,5-tetramethyl-2-(4,4,5,5-tetramethyl-1,3,2-dioxaborolan-2-yl)-1,3,2-dioxaborolane (36.01 g, 141.80 mmol) in dioxane (400 mL) was added potassium acetate (27.83 g, 283.60 mmol) at room temperature. The reaction mixture was degassed with argon for 10 minutes and cyclopentyl(diphenyl)phosphane; dichloromethane; dichloropalladium; iron (3.86 g, 4.73 mmol) was added at room temperature. The reaction mixture was degassed with argon again for 5 minutes and the reaction mixture was stirred at 100° C. for 16 hours. Progress of the reaction was monitored by LC-MS. After completion of the reaction, the reaction mixture was concentrated under reduced pressure to get the crude product, which was purified by column chromatography (Davisil silica, 5% ethyl acetate in pet ether) to afford 2,6-dibenzyloxy-3-(4,4,5,5-tetramethyl-1,3,2-dioxaborolan-2-yl)pyridine (30 g, 38.21 mmol, 40.42% yield) as a light green gummy. LC-MS (ES⁺): m/z 418.53 [M+H]⁺.

### Step-3:

To a stirred solution of ethyl 2-[2-(4-bromophenyl)-2-azaspiro[3.3]heptan-6-yl]acetate (0.5 g, 1.48 mmol) in 1,4-dioxane (10 mL) and water (2 mL) was added sodium tert-butoxide (142.06 mg, 1.48 mmol), and the reaction mixture was degassed for 15 minutes. Then [1,1′-bis(diphenylphosphino)ferrocene]dichloropalladium(II) (216.33 mg, 295.65 μmol) was added slowly and the reaction mixture was stirred at 100° C. for 16 hours. After completion of the reaction as confirmed by LC-MS, the reaction mixture was filtered through celite pad and concentrated under reduced pressure at 50° C. The resulting crude was purified by flash column chromatography (silica gel 100-200 mesh, 0-30% methanol in DCM) to afford ethyl 2-[2-[4-(2,6-dibenzyloxy-3-pyridyl)phenyl]-2-azaspiro[3.3]heptan-6-yl]acetate (0.35 g, 603.08 μmol, 40.80% yield). LC-MS (ES⁺): m/z 549.32 [M+H]⁺.

### Step-4:

To a stirred solution of ethyl 2-[2-[4-(2,6-dibenzyloxy-3-pyridyl)phenyl]-2-azaspiro[3.3]heptan-6-yl]acetate (0.2 g, 364.52 μmol) in THE (10 mL) and ethanol (10 mL) was added 10% palladium on carbon (193.96 mg, 1.82 mmol). The reaction mixture was stirred at room temperature for 16 hours while monitoring by LC-MS. Upon completion of the reaction, the reaction mixture was filtered through celite pad and then concentrated under reduced pressure at 45° C. to afford ethyl 2-[2-[4-(2,6-dioxo-3-piperidyl)phenyl]-2-azaspiro[3.3]heptan-6-yl]acetate (0.2 g, 355.36 μmol, 97.49% yield). LC-MS (ES⁺): m/z 371.14 [M+H]⁺.

### Step-5:

To a stirred solution of ethyl 2-[2-[4-(2,6-dioxo-3-piperidyl)phenyl]-2-azaspiro[3.3]heptan-6-yl]acetate (0.15 g, 404.92 μmol) in DCE (20 mL) was added trimethyltin hydroxide (439.31 mg, 2.43 mmol) and the reaction mixture was stirred at 100° C. for 16 hours. After completion of the reaction as confirmed by LC-MS, the reaction mixture was quenched with HCl in dioxane (0.5 mL) and concentrated under reduced pressure at 50° C. to afford 2-[2-[4-(2,6-dioxo-3-piperidyl)phenyl]-2-azaspiro[3.3]heptan-6-yl]acetic acid (0.2 g, 277.93 μmol, 68.64% yield). LC-MS (ES⁺): m/z 343.44 [M+H]⁺.

### Synthesis of 2-[4-[3-(2,6-dioxo-3-piperidyl)-1-methyl-indazol-6-yl]-3,3-difluoro-1-piperidyl]acetic acid

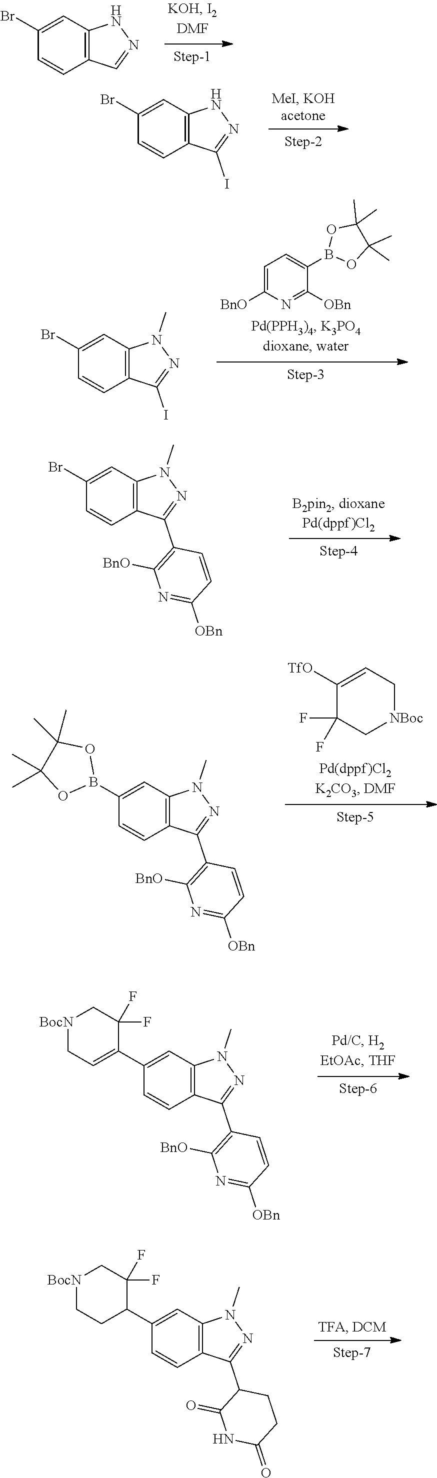

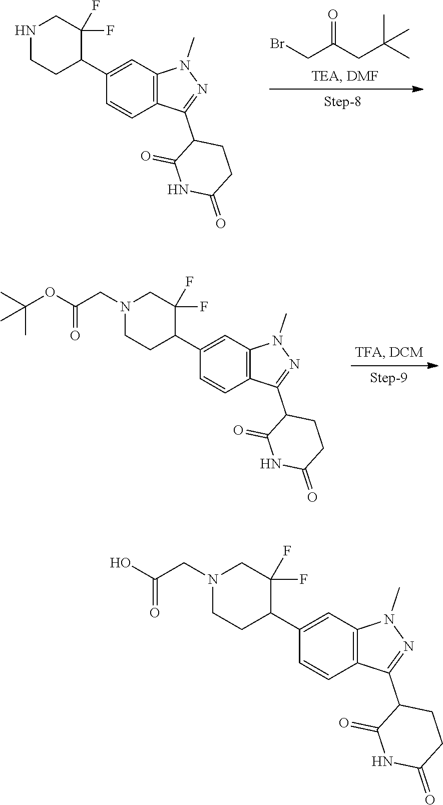

### Step-1:

To a suspension of 6-bromo-1H-indazole (15 g, 76.13 mmol) in DMF (120.00 mL) was added KOH (10.25 g, 182.71 mmol) at 0° C. portion-wise over a period of 10 minutes and the reaction mixture was stirred at room temperature for 15 minutes. Iodine (19.63 g, 153.47 mmol) was added and the reaction mixture was stirred at room temperature for 3 hours. The progress of reaction was monitored by TLC and LC-MS. The reaction mixture was partitioned between ethyl acetate and a 1:1 mixture of aqueous saturated NaCl and saturated sodium thiosulfate. The aqueous layer was extracted with ethyl acetate. The combined organic layer was washed with water and brine, dried over anhydrous sodium sulfate and concentrated under reduced pressure to afford 6-bromo-3-iodo-1H-indazole (19.5 g, 57.88 mmol, 76.03% yield) as a light yellow solid. LC-MS (ES⁺): m/z 323.19 [M+H]⁺.

### Step-2:

To a stirred solution of 6-bromo-3-iodo-1H-indazole (19 g, 58.84 mmol) in Acetone (200.19 mL) under room temperature under nitrogen atmosphere. The reaction mixture was cooled to 0° C. then potassium hydroxide (4.95 g, 88.25 mmol) was added and maintained at the same temperature. The reaction mixture was stirred for 30 minutes at this temperature. To the reaction mixture was added iodomethane (8.35 g, 58.84 mmol, 3.66 mL) drop wise. The reaction mixture was warmed to room temperature and then stirred for 3 hours. The progress of reaction was monitored by TLC and LC-MS. After completion of the reaction, the solvent was removed under reduced pressure, washed with ethyl acetate, and filtered. The filtrate was concentrated under reduced pressure and the crude product was purified (60-120 mesh silica gel, 30% ethyl acetate in hexane) to afford 6-bromo-3-iodo-1-methyl-indazole (10.5 g, 29.99 mmol, 50.97% yield) as yellow color solid. LC-MS (ES⁺): m/z 337.22 [M+H]⁺.

### Step-3:

A solution of 6-bromo-3-iodo-1-methyl-indazole (3 g, 8.90 mmol), 2,6-dibenzyloxy-3-(4,4,5,5-tetramethyl-1,3,2-dioxaborolan-2-yl)pyridine (7.43 g, 17.81 mmol) in 1,4 dioxane (85 mL), water (30 mL) at room temperature was degassed with argon for 10 minutes. To the reaction mixture were added tripotassium phosphate (5.67 g, 26.71 mmol) and palladium; triphenylphosphane (617.30 mg, 534.20 μmol) at same temperature. The reaction mixture was degassed with argon for another 10 minutes, and was then stirred at 110° C. for 4 hours. The reaction progress was monitored by TLC and LC-MS. After completion, the reaction mixture was concentrated under reduced pressure and extracted with ethyl acetate and water. The organic layer was washed with brine solution, dried over sodium sulfate and concentrated under reduced pressure. The crude product was purified by column chromatography (Davisil silica) to afford 6-bromo-3-(2,6-dibenzyloxy-3-pyridyl)-1-methyl-indazole (1.5 g, 2.92 mmol, 32.75% yield) as a light yellow solid. LC-MS (ES⁺): m/z 500.40 [M+H]⁺.

### Step-4:

To a stirred solution of 6-bromo-3-(2,6-dibenzyloxy-3-pyridyl)-1-methyl-indazole (1 g, 2.00 mmol) and 4,4,5,5-tetramethyl-2-(4,4,5,5-tetramethyl-1,3,2-dioxaborolan-2-yl)-1,3,2-dioxaborolane (507.48 mg, 2.00 mmol) in 1,4 dioxane (10 mL) was added potassium acetate (588.40 mg, 6.00 mmol) at room temperature. The reaction mixture was degassed with argon for 5 minutes and cyclopentyl(diphenyl)phosphane; dichloromethane; dichloropalladium;iron (97.92 mg, 119.91 μmol) was added to the reaction mixture. The reaction mixture was degassed with argon for 1 minute before it was stirred at 90° C. for 16 hours. The progress of reaction was monitored by LC-MS. After completion of reaction, the reaction mixture was filtered through celite bed and bed was washed with dioxane. The organic layer was concentrated under reduced pressure and the obtained residue was purified by column chromatography (Davisil silica, 40% ethyl acetate in pet-ether) to afford 3-(2,6-dibenzyloxy-3-pyridyl)-1-methyl-6-(4,4,5,5-tetramethyl-1,3,2-dioxaborolan-2-yl) indazole (0.9 g, 1.52 mmol, 75.94% yield) as a brown solid. LC-MS (ES⁺): m/z 548.45 [M+H]⁺.

### Step-5:

To a stirred solution of tert-butyl 3,3-difluoro-4-(trifluoromethylsulfonyloxy)-2,6-dihydropyridine-1-carboxylate (369.00 mg, 1.00 mmol) and 3-(2,6-dibenzyloxy-3-pyridyl)-1-methyl-6-(4,4,5,5-tetramethyl-1,3,2-dioxaborolan-2-yl) indazole (0.5 g, 913.32 μmol) in DMF (5 mL) was added potassium carbonate, granular (378.69 mg, 2.74 mmol), cyclopentyl(diphenyl)phosphane; dichloropalladium; iron (40.10 mg, 54.80 μmol). The reaction mixture was degassed with argon for 15 minutes at room temperature and was then stirred at 85° C. for 3 hours. The progress of reaction was monitored by LC-MS. After completion of the reaction, the reaction mixture was filtered through celite bed and washed with dichloromethane (3×20 mL). The combined organic layer was washed with brine solution (10 mL) and concentrated under reduced pressure at 45° C. to get crude product. The crude product was purified by column chromatography (Davisil silica, 10% ethyl acetate in pet-ether) to afford tert-butyl 4-[3-(2,6-dibenzyloxy-3-pyridyl)-1-methyl-indazol-6-yl]-3,3-difluoro-2,6-dihydropyridine-1-carboxylate (0.5 g, 682.09 μmol, 74.68% yield) as a yellow gummy liquid. LC-MS (ES⁺): m/z 639.97 [M+H]⁺.

### Step-6:

To a stirred solution of tert-butyl 4-[3-(2,6-dibenzyloxy-3-pyridyl)-1-methyl-indazol-6-yl]-3,3-difluoro-2,6-dihydropyridine-1-carboxylate (0.51 g, 798.50 μmol) in EtOAc (10 mL) and THF (10 mL) was degassed with nitrogen gas for 10 minutes and palladium on carbon (934.73 mg, 8.78 mmol) was added at room temperature. The reaction mixture was stirred under hydrogen atmosphere (balloon pressure) at room temperature for 16 hours. Progress of reaction was monitored by TLC and LC-MS. After completion of the reaction, the reaction mixture was filtered through celite bed and washed with THE (50 mL) and EtOAc (50 mL). The filtrate was concentrated under reduced pressure to afford tert-butyl 4-[3-(2,6-dioxo-3-piperidyl)-1-methyl-indazol-6-yl]-3,3-difluoro-piperidine-1-carboxylate (0.35 g, 556.53 μmol, 69.70% yield) as a colorless gum. LC-MS (ES⁺): m/z 463.48 [M+H]⁺.

### Step-7:

To a stirred solution of tert-butyl 4-[3-(2,6-dioxo-3-piperidyl)-1-methyl-indazol-6-yl]-3,3-difluoro-piperidine-1-carboxylate (0.220 g, 475.69 μmol) in DCM (10.48 mL) was added TFA (296.00 mg, 2.60 mmol, 0.2 mL) at 0° C. and continued stirring for 4 hours at room temperature. The reaction progress was monitored by LC-MS. After completion of the reaction, solvent was evaporated under vacuum to obtain crude product. The crude was triturated in diethyl ether (10 mL), and the formed solid was filtered and dried to afford 3-[6-(3,3-difluoro-4-piperidyl)-1-methyl-indazol-3-yl]piperidine-2,6-dione TFA salt (0.180 g, 324.94 μmol, 68.31% yield) as a grey color solid. LC-MS (ES⁺): m/z 363.43 [M+H]⁺.

### Step-8:

To a solution of 3-[6-(3,3-difluoro-4-piperidyl)-1-methyl-indazol-3-yl]piperidine-2,6-dione (75 mg, 206.97 μmol) and tert-butyl 2-bromoacetate (44.41 mg, 227.67 μmol, 33.39 μL) in DMF (2 mL) was added TEA (167.55 mg, 1.66 mmol, 230.78 μL). The mixture was stirred at 25° C. for 16 hours. After consumption of the reactant as confirmed by LC-MS, the mixture was diluted with water (20 mL) and extracted with ethyl acetate (15 mL×3). The combined organic layers were washed with brine (10 mL), dried over anhydrous sodium sulfate, filtered, and concentrated under reduced pressure. The residue was purified by column chromatography (silica gel, petroleum ether/ethyl acetate=50/1 to 1/1). Compound tert-butyl 2-(4-(3-(2,6-dioxopiperidin-3-yl)-1-methyl-1H-indazol-6-yl)-3,3-difluoropiperidin-1-yl)acetate (52 mg, 87.30 μmol, 42.18% yield) was obtained as a light yellow oil. LC-MS (ES⁺): m/z 477.2 [M+H]⁺.

### Step-9:

To a stirred solution of tert-butyl 2-[4-[3-(2,6-dioxo-3-piperidyl)-1-methyl-indazol-6-yl]-3,3-difluoro-1-piperidyl]acetate (0.130 g, 272.81 μmol) in DCM (5 mL) under nitrogen atmosphere, 2,2,2-trifluoroacetic acid (311.07 mg, 2.73 mmol, 210.18 μL) was added at 0° C. and then the reaction mixture was stirred for 1 hour at 25° C. After completion of the reaction, DCM was evaporated under vacuum. The crude material was triturated with diethyl ether to afford 2-[4-[3-(2,6-dioxo-3-piperidyl)-1-methyl-indazol-6-yl]-3,3-difluoro-1-piperidyl]acetic acid (88 mg, 148.62 μmol, 54.48% yield) as an off-white solid. LC-MS (ES⁺): m/z 421.80 [M+H]⁺.

### Synthesis of 2-[1-[3-(2,6-dioxo-3-piperidyl)-1-methyl-indazol-6-yl]-4-piperidyl]acetic acid Br N

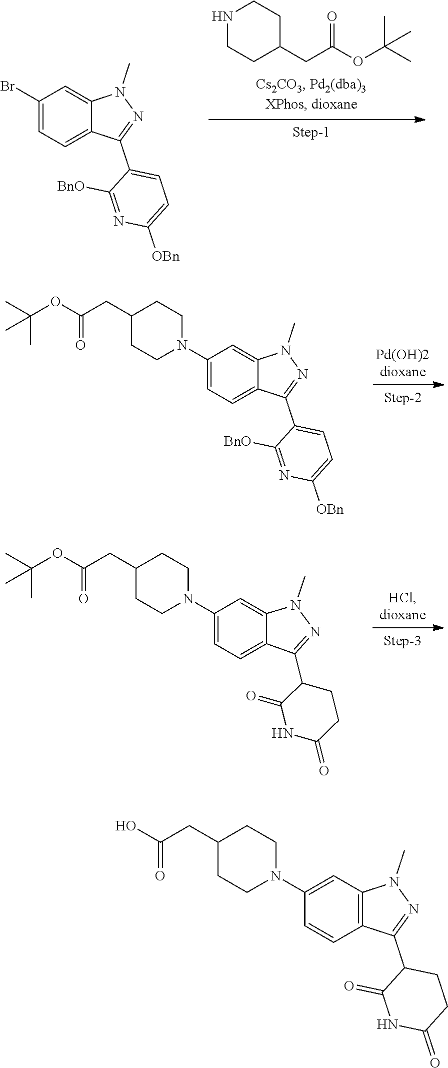

### Step-1:

In a sealed tube, a solution of 6-bromo-3-(2,6-dibenzyloxy-3-pyridyl)-1-methyl-indazole (2 g, 3.68 mmol) in Dioxane (20 mL) were added tris(dibenzylideneacetone) dipalladium(0) (202.04 mg, 220.63 μmol) and X-Phos (175.30 mg, 367.72 μmol). The resulting solution was purged with nitrogen gas for 20 minutes, then added cesium carbonate (3.59 g, 11.03 mmol). The sealed reaction mixture was stirred at 100° C. temperature for 16 hours. The progress of the reaction was monitored by LC-MS and TLC. After completion of the reaction, the reaction mixture was filtered through celite bed, washed with ethyl acetate (200 mL) and concentrated under reduced pressure to get the crude product, which was purified by column chromatography (silica gel, 40% ethyl acetate and 60% pet ether) to afford tert-butyl 2-[1-[3-(2,6-dibenzyloxy-3-pyridyl)-1-methyl-indazol-6-yl]-4-piperidyl]acetate (1.82 g, 2.36 mmol, 64.15% yield) as an off-white solid. LC-MS (ES⁺): m/z 619.2 [M+H]⁺.

### Step-2:

To a stirred solution of tert-butyl 2-[1-[3-(2,6-dibenzyloxy-3-pyridyl)-1-methyl-indazol-6-yl]-4-piperidyl]acetate (2 g, 3.23 mmol) in 1,4-dioxane (30 mL) purged with nitrogen gas was added palladium hydroxide on carbon, 20 wt. % 50% water (10.78 mg, 76.73 μmol). The reaction mixture was stirred under hydrogen atmosphere at room temperature for 16 hours. The progress of the reaction was monitored by LC-MS. After completion of the reaction, the reaction mixture was filtered through celite bed, washed with ethyl acetate (200 mL) and concentrated under reduced pressure to get the product tert-butyl 2-[1-[3-(2,6-dioxo-3-piperidyl)-1-methyl-indazol-6-yl]-4-piperidyl]acetate (1.4 g, 3.03 mmol, 93.80% yield) as an off-white solid. LC-MS (ES⁺): m/z 441.2 [M+H]⁺.

### Step-3:

To a stirred solution of tert-butyl 2-[1-[3-(2,6-dioxo-3-piperidyl)-1-methyl-indazol-6-yl]-4-piperidyl]acetate (1.4 g, 3.03 mmol) in 1,4-dioxane (20 mL) cooled to 0° C. was added 4.0 M hydrogen chloride solution in dioxane (757.94 mmol) dropwise and stirred at room temperature for 16 hours. The progress of the reaction was monitored by LC-MS. After completion of the reaction, the reaction mixture was directly concentrated, washed with hexane (100 mL) and dried to get the product 2-[1-[3-(2,6-dioxo-3-piperidyl)-1-methyl-indazol-6-yl]-4-piperidyl]acetic acid HCl salt (1.25 g, 2.58 mmol, 85.22% yield) as an off-white solid. LC-MS (ES⁺): m/z 385.2 [M+H]⁺.

### Synthesis of 2-[1-[3-[(3R)-2,6-dioxo-3-piperidyl]-1-methyl-indazol-6-yl]-4-piperidyl]acetic acid and 2-[1-[3-[(3S)-2,6-dioxo-3-piperidyl]-1-methyl-indazol-6-yl]-4-piperidyl]acetic acid

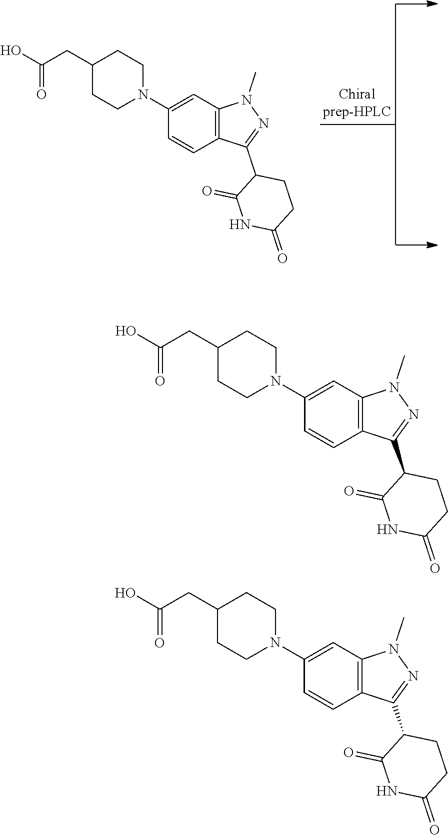 [SMILES: Cn1nc(C2CCC(=O)NC2=O)c2ccc(N3CCC(CC(=O)O)CC3)cc21 ; Cn1nc([C@H]2CCC(=O)NC2=O)c2ccc(N3CCC(CC(=O)O)CC3)cc21 ; Cn1nc([C@@H]2CCC(=O)NC2=O)c2ccc(N3CCC(CC(=O)O)CC3)cc21]

Chiral separation of 2-[1-[3-(2,6-dioxo-3-piperidyl)-1-methyl-indazol-6-yl]-4-piperidyl]acetic acid (500 mg, 1.30 mmol) by normal phase chiral prep HPLC afforded 2-[1-[3-[(3R)-2,6-dioxo-3-piperidyl]-1-methyl-indazol-6-yl]-4-piperidyl]acetic acid (120 mg, 305.10 μmol, 23.46% yield) and 2-[1-[3-[(3S)-2,6-dioxo-3-piperidyl]-1-methyl-indazol-6-yl]-4-piperidyl]acetic acid (100 mg, 258.59 μmol, 19.88% yield) using the method below.

Column: Chiralpak IC (250×21 mm) 5

Mobile Phase: DCM/IPA: 60/40

Flow rate: 18 ml/min

Run time: 18.0 min.

Wave length: 250 nm

Solubility: DCM+TFE

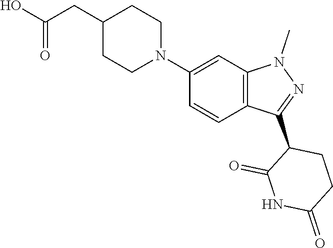 [SMILES: Cn1nc([C@H]2CCC(=O)NC2=O)c2ccc(N3CCC(CC(=O)O)CC3)cc21]

### 2-[1-[3-[(3R)-2,6-dioxo-3-piperidyl]-1-methyl-indazol-6-yl]-4-piperidyl]acetic acid

¹H NMR (400 MHz, DMSO-d₆) δ 12.10 (bs, 1H), 10.85 (s, 1H), 7.48 (d, J=8.4 Hz, 1H), 6.90 (d, J=8.3 Hz, 1H), 6.84 (bs, 1H), 4.26-4.23 (m, 1H), 3.88 (s, 3H), 3.76 (d, J=11.8 Hz, 2H), 2.77-2.68 (m, 2H), 2.63-2.59 (m, 2H), 2.32-2.27 (m, 1H), 2.21-2.13 (m, 3H), 1.84-1.76 (m, 3H), 1.35-1.33 (m, 2H).

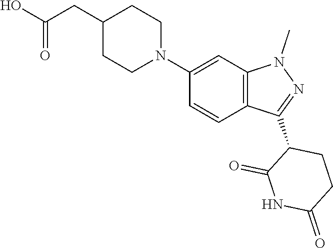 [SMILES: Cn1nc([C@@H]2CCC(=O)NC2=O)c2ccc(N3CCC(CC(=O)O)CC3)cc21]

### 2-[1-[3-[(3S)-2,6-dioxo-3-piperidyl]-1-methyl-indazol-6-yl]-4-piperidyl]acetic acid

¹H NMR (400 MHz, DMSO-d₆) δ 12.10 (bs, 1H), 10.85 (s, 1H), 7.48 (d, J=8.9 Hz, 1H), 6.91 (d, J=7.8 Hz, 1H), 6.84 (bs, 1H), 4.26-4.23 (m, 1H), 3.88 (s, 3H), 3.77-3.75 (m, 2H), 2.77-2.68 (m, 2H), 2.63-2.56 (m, 2H), 2.32-2.27 (m, 1H), 2.21-2.13 (m, 3H), 1.84-1.76 (m, 3H), 1.35-1.33 (m, 2H).

### 3-((5-fluoro-6-(piperidin-4-yl)pyridin-3-yl)amino)piperidine-2,6-dione

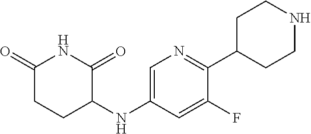 [SMILES: O=C1CCC(Nc2cnc(C3CCNCC3)c(F)c2)C(=O)N1]

This compound was prepared according to the method described on page 706 of WO2021/127561A1.

### Synthesis of 3-[4-(3-piperidyl)phenoxy]piperidine-2,6-dione

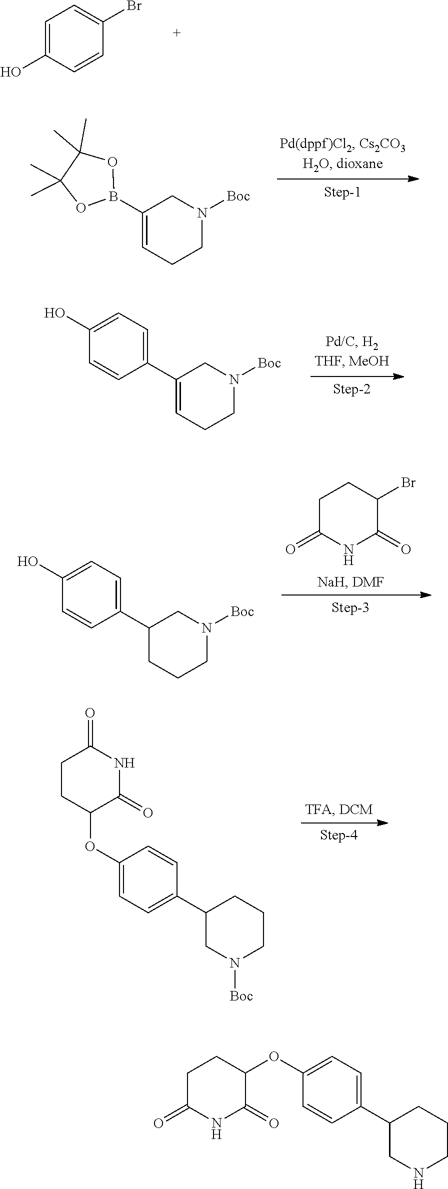

### Step-1:

To a stirred solution of 4-bromophenol (5 g, 28.90 mmol) in water (2 mL) and dioxane (25 mL) was added tert-butyl 5-(4,4,5,5-tetramethyl-1,3,2-dioxaborolan-2-yl)-3,6-dihydro-2H-pyridine-1-carboxylate (8.94 g, 28.90 mmol) and cesium carbonate (28.25 g, 86.70 mmol). The reaction mixture was degassed with argon for 10 minutes before [1,1′-bis(diphenylphosphino)ferrocene]dichloropalladium(II), complex with dichloromethane (1.89 g, 2.31 mmol) was added and the resulting mixture was stirred at 100° C. for 16 h. Upon completion of the reaction, the reaction mixture was cooled to room temperature and filtered through a short bed of celite. The filtrate was diluted with ethyl acetate (2×150 mL), washed with water (30 mL), dried over anhydrous Na₂SO₄ and concentrated under reduced pressure. The crude product was purified by column chromatography (silica gel, 0-30% ethyl acetate in hexane) to afford tert-butyl 5-(4-hydroxyphenyl)-3,6-dihydro-2H-pyridine-1-carboxylate (4.5 g, 14.38 mmol, 49.76% yield) as an off-white solid. LCMS (ES⁻): m/z 274.32 [M−H]⁻.

### Step-2:

In a round bottom flask, to a stirred solution of tert-butyl 5-(4-hydroxyphenyl)-3,6-dihydro-2H-pyridine-1-carboxylate (2.5 g, 9.08 mmol) in THF (10 mL), methanol (10 mL) was added 10% Palladium on carbon wet (2.50 g, 23.49 mmol) and the reaction was stirred under hydrogen atmosphere at 25° C. for 16 h. Upon completion of the reaction, the reaction mixture was filtered through celite bed, and washed with 10% methanol and dichloromethane (70 mL). The filtrate and concentrated under reduced pressure to give tert-butyl 3-(4-hydroxyphenyl)piperidine-1-carboxylate (2.4 g, 7.70 mmol, 84.82% yield). LCMS (ES⁻): m/z 275.81 [M−H]⁻.

### Step-3:

To a stirred solution of tert-butyl 3-(4-hydroxyphenyl)piperidine-1-carboxylate (2 g, 7.21 mmol) in DMF (50 mL) was added sodium hydride (60% dispersion in mineral oil) (904.25 mg, 21.63 mmol) slowly at 0° C. It was then stirred at rt for 1 h after which 3-bromopiperidine-2,6-dione (4.15 g, 21.63 mmol) was added slowly at 0° C. The reaction was stirred at rt for another 6 h. Upon completion of the reaction, the reaction mixture was quenched with ice water (15 vol) and extracted with ethyl acetate (3×30 vol). The combined organic layers were dried over anhydrous Na₂SO₄ and concentrated in vacuo. The crude compound was purified by reverse phase column chromatography (Reveleris C18 80 g, 0-60% 0.05% ammonium Bicarbonate in water/ACN) to afford tert-butyl 3-[4-[(2,6-dioxo-3-piperidyl)oxy]phenyl]piperidine-1-carboxylate (1.3 g, 2.01 mmol, 27.85% yield) as off white solid. LCMS (ES⁻): m/z 387.37 [M−H]⁻.

### Step-4:

To a stirred solution of tert-butyl 3-[4-[(2,6-dioxo-3-piperidyl)oxy]phenyl]piperidine-1-carboxylate (0.050 g, 128.71 μmol) in DCM (3.86 mL) was added trifluoroacetic acid (14.68 mg, 128.71 μmol, 9.92 μL) and stirred at rt for 3 h. Upon completion of the reaction, the reaction mixture was concentrated in vacuo and the residue was triturated with diethyl ether to afford 3-[4-(3-piperidyl)phenoxy]piperidine-2,6-dione (0.04 g, 94.44 μmol, 73.37% yield, TFA salt) as an off white solid. LCMS (ES⁺): m/z 289.3 [M+H]⁺.

### Synthesis of 3-[4-[[(3S)-morpholin-3-yl]methyl]phenoxyl piperidine-2,6-dione

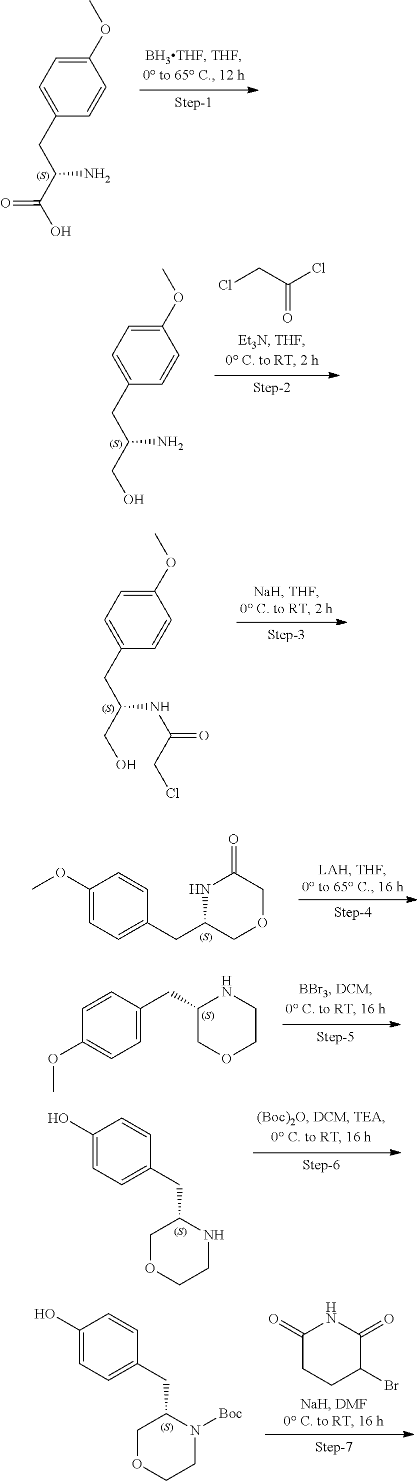

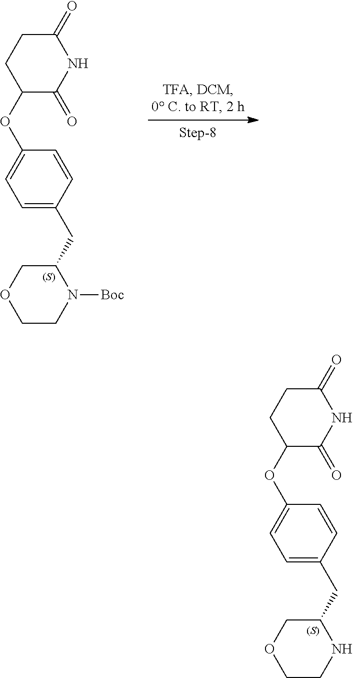

### Step-1:

A stirred solution of (S)-2-amino-3-(4-methoxyphenyl)propanoic acid (40.0 g, 204.9 mmol) in THF (609.8 mL) was cooled to 0° C. and borane;tetrahydrofuran (1 M, 1.02 L) was added dropwise. After addition, the reaction mixture was allowed to stir at 65° C. for 12 h. After completion of the reaction, the reaction mixture was diluted with methanol at 0° C. The mixture was concentrated under reduced pressure, and the obtained residue was diluted with saturated sodium bicarbonate solution (200 mL) and extracted with 10% methanol in DCM (3×250 mL). The organic layer ware separated, dried over anhydrous sodium sulfate and evaporated under reduced pressure to give the crude product, which was triturated with diethyl ether (200 mL) to afford (S)-2-amino-3-(4-methoxyphenyl)propan-1-ol (18.5 g, 98.07 mmol, 47.86% yield) as a white solid. LCMS (ES⁺): m/z 182.34 [M+H]⁺

### Step-2:

A stirred solution of (S)-2-amino-3-(4-methoxyphenyl)propan-1-ol (18.5, 102.08 mmol) in THF (800 mL) was cooled to 0° C. before TEA (30.99 g, 606.24 mmol, 42.68 mL) and chloroacetyl chloride, 98% (11.53 g, 102.08 mmol, 8.14 mL) were added dropwise. The reaction was stirred at room temperature for 2 h. After completion of the reaction, the reaction mixture was concentrated under reduced pressure to give the crude product, which was triturated with diethyl ether (100 mL) to afford (S)-2-chloro-N-(1-hydroxy-3-(4-methoxyphenyl)propan-2-yl)acetamide (4) (26 g, 30.1 mmol, 29.49% yield) as an orange solid. LCMS (ES⁻): m/z 256.33 [M−H]⁻

### Step-3:

To a stirred solution of (S)-2-chloro-N-(1-hydroxy-3-(4-methoxyphenyl)propan-2-yl)acetamide (18.0 g, 69.85 mmol) in THF (700 mL) was added sodium hydride (60% dispersion in mineral oil) (4.82 g, 209.54 mmol) portionwise at 0° C. over a period of 10 min. The reaction mixture was stirred at room temperature for 2 h. After completion of the reaction, the reaction mixture was quenched with cold water (100 mL) and extracted with ethyl acetate (3×200 mL). The combined organic layer was separated, dried over sodium sulfate and concentrated under reduced pressure to give the crude product (19 g), which was purified by column chromatography (Davisil-silica) using 50% ethyl acetate in pet ether as eluent to afford (S)-5-(4-methoxybenzyl)morpholin-3-one (9.5 g, 42.94 mmol, 61.48% yield) as an off-white solid. LCMS (ES⁺): m/z 222.2 [M+H]⁺

### Step-4:

A stirred solution of (S)-5-(4-methoxybenzyl)morpholin-3-one (9.5 g, 42.94 mmol) in THF (200 mL) was cooled to 0° C. and lithium aluminium hydride (2M, 150.28 mL) was added dropwise. The reaction mixture to stirred at room temperature for 10 min and then heated at 65° C. for 16 h. After completion of the reaction, the reaction mixture was quenched with cold aqueous saturated ammonium chloride solution (100 mL) and extracted with ethyl acetate (3×200 mL). The combined organic layer was separated, dried over sodium sulfate and concentrated under reduced pressure to give the crude product (13 g), which was triturated with diethyl ether to afford (S)-3-(4-methoxybenzyl)morpholine (9.0 g, 37.38 mmol, 86.05% yield) as a gummy liquid. LCMS (ES⁺): m/z 208.24 [M+H]⁺

### Step-5:

To a stirred solution of (S)-3-(4-methoxybenzyl)morpholine (9.0 g, 43.42 mmol) in DCM (250 mL) was added tribromoborane (1M, 219.17 mL) dropwise at 0° C. The reaction mixture was stirred at RT for 16 h. After completion of the reaction, the reaction mixture was quenched with cold aqueous saturated sodium bicarbonate solution (200 mL) at 0° C. until pH=8. The basic mixture was extracted with 10% methanol in DCM (3×200 mL). The organic layer was separated and dried over anhydrous sodium sulfate and concentrated under reduced pressure to obtain the crude product, which was triturated with diethyl ether (100 mL) to afford (S)-4-(morpholin-3-ylmethyl)phenol (7) (9.0 g, 39.3 mmol, 39.30% yield) as a brown gummy oil. LCMS (ES⁺): m/z 194.28 [M+H]⁺

### Step-6:

To a stirred solution of (S)-4-(morpholin-3-ylmethyl)phenol (9.0 g, 46.57 mmol) in DCM (200 mL) and TEA (7.07 g, 69.86 mmol, 9.74 mL) was added tert-butoxycarbonyl tert-butyl carbonate (10.16 g, 46.57 mmol, 10.69 mL) dropwise at 0° C. The reaction mixture was stirred at RT for 16 h. After completion of the reaction, the reaction mixture was quenched with cold water (100 mL) and extracted with DCM (3×200 mL). The organic layer was separated and dried over anhydrous sodium sulphate and concentrated under reduced pressure to obtain crude oil. The obtained crude was purified by column chromatography (Davisil-silica) using 30% ethyl acetate in pet ether to afforded tert-butyl (S)-3-(4-hydroxybenzyl)morpholine-4-carboxylate (7.0 g, 23.06 mmol, 49.5% yield) as colourless gummy oil. LCMS (ES⁺): m/z 292.70 [M+H]⁺

### Step-7:

To a stirred solution of tert-butyl (S)-3-(4-hydroxybenzyl)morpholine-4-carboxylate (7.0 g, 23.86 mmol) in DMF (25 mL) was added sodium hydride (60% dispersion in mineral oil (1.37 g, 59.65 mmol) and stirred the reaction mixture at 0° C. for 30 min. Added 3-bromopiperidine-2,6-dione (6.87 g, 35.79 mmol) and stirred the reaction mixture at 28° C. for 16 h. After completion of the reaction, the reaction mixture was quenched with cold aqueous saturated ammonium chloride solution (100 mL) and the reaction mixture was extracted with ethyl acetate (3×30 mL). The combined organic layer was separated, dried over sodium sulfate and concentrated under reduced pressure to give the crude product (10 g), which was purified by column chromatography (Davisil-silica) using 30% ethyl acetate in pet ether as eluent to afford tert-butyl (3S)-3-(4-((2,6-dioxopiperidin-3-yl)oxy)benzyl)morpholine-4-carboxylate (5.33 g, 13.09 mmol, 54.86% yield) as an off-white solid. LCMS (ES⁻): m/z 403.31 [M−H]⁻

### Step-8:

To a stirred solution of tert-butyl (3S)-3-(4-((2,6-dioxopiperidin-3-yl)oxy)benzyl)morpholine-4-carboxylate (1.0 g, 2.47 mmol) in DCM (20 mL) was added trifluoracetic acid, 99% (5.64 g, 49.45 mmol, 3.81 mL) dropwise at 0° C. The reaction mixture was stirred at 25° C. for 2 h. Upon completion of the reaction, the reaction mixture was concentrated under reduced pressure to get crude product (1 g), which was triturated with pet ether (50 mL) and pentane (50 mL) to afford 3-[4-[[(3S)-morpholin-3-yl]methyl]phenoxy]piperidine-2,6-dione (0.8 g, 704.22 μmol, 28.48% yield, HCl salt) as white solid. LCMS (ES⁺): m/z 305.39 [M+H]⁺

### 3-[4-[[(3R)-morpholin-3-yl]methyl]phenoxy]piperidine-2,6-dione

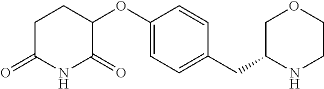 [SMILES: O=C1CCC(Oc2ccc(C[C@@H]3COCCN3)cc2)C(=O)N1]

This compound was prepared substantially following the synthesis of 3-[4-[[(3S)-morpholin-3-yl]methyl]phenoxy]piperidine-2,6-dione, using (2R)-2-amino-3-(4-methoxyphenyl)propanoic acid as starting material. LCMS (ES⁺): m/z 305.19 [M+H]⁺.

### Synthesis of 3-[4-(2-piperidylmethyl)phenoxy]piperidine-2,6-dione

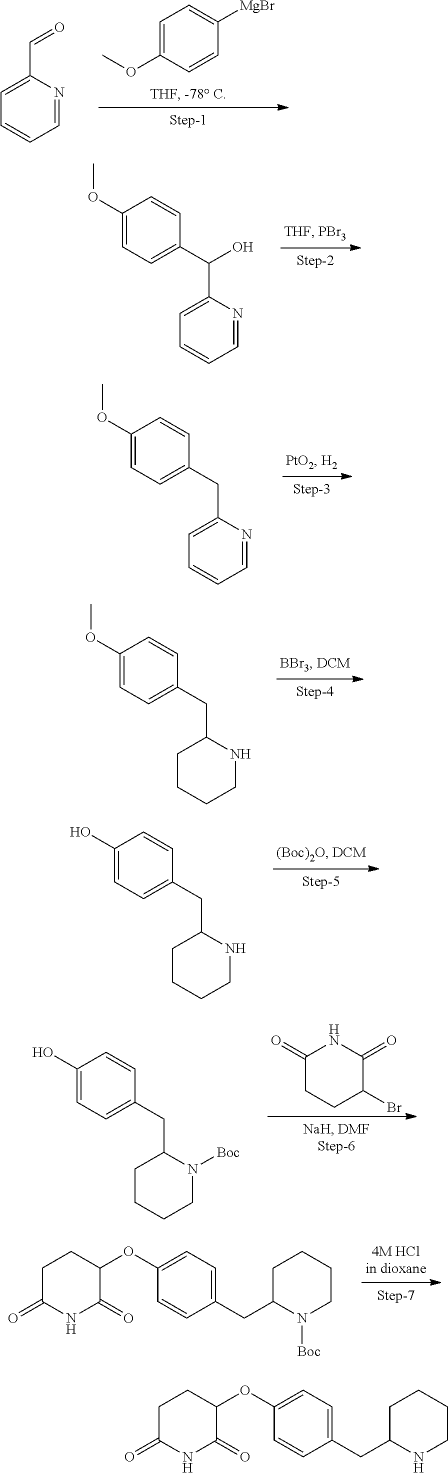

### Step-1:

To a solution of pyridine-2-carbaldehyde (25 g, 233.41 mmol) in THF (500 mL) was added bromo-(4-methoxyphenyl)magnesium (1 M, 350.11 mL, 350 mmol) dropwise at −78° C. The reaction mixture was stirred at room temperature for 4 h. After completion of the reaction, the reaction mixture was quenched by the addition of saturated aqueous ammonium chloride (500 mL), and then extracted with ethyl acetate (2×250 mL). The combined organic layer was washed with brine (250 mL), and dried over Na₂SO₄. The mixture was concentrated in vacuo and triturated with pentane to give (4-methoxyphenyl)-(2-pyridyl) methanol (35 g, 139.66 mmol, 59.84% yield) as a pale yellow solid. LCMS (ES⁺): m/z 238.18 [M+Na]⁺

### Step-2:

To the mixture of (4-methoxyphenyl)-(2-pyridyl)methanol (35 g, 162.60 mmol) in THE (500 mL) was added phosphorus tribromide (132.05 g, 487.81 mmol, 45.85 mL). The mixture was stirred at room temperature for 0.5 h, and then heated at 80° C. for 4 h. After the completion of the reaction, after cooling in an ice water bath, the reaction was quenched by adding water. The pH of the mixture was adjusted to 11 by the addition of saturated sodium carbonate, and the mixture was then extracted with ethyl acetate (3×500 mL). The organic layer was dried over anhydrous Na₂SO₄ and concentrated in vacuo. The crude material was purified by flash column chromatography on 100-200 mesh silica gel (Hexane:AcOEt=3:1) to give 2-[(4-methoxyphenyl)methyl]pyridine (18 g, 65.38 mmol, 40.21% yield) as a pale yellow oil. LCMS (ES⁺): m/z 200.19 [M+H]⁺.

### Step-3:

To a stirred solution of 2-[(4-methoxyphenyl)methyl]pyridine (18 g, 90.34 mmol) in acetic acid (180 mL) was added platinum dioxide (2.05 g, 9.03 mmol) and hydrogenated in a parr-shaker at 30 psi for for 48 hr. After completion of the reaction, the reaction mixture was filtered through celite and the celite bed was washed with ethyl acetate (500 mL) and concentrated to get crude compound 2-[(4-methoxyphenyl)methyl]piperidine (18 g, 75.93 mmol, 84.05% yield) as pale brown oil. LCMS (ES⁺): m/z 206.13 [M+H]⁺.

### Step-4:

To a stirred solution of 2-[(4-methoxyphenyl)methyl]piperidine (9 g, 43.84 mmol) in DCM (90 mL), tribromoborane (1.0 M, 87.68 mL) was added at rt and the reaction was stirred at room temperature for 16 h. After consumption of the starting material, the reaction mixture was quenched with methanol at 0° C. and stirred for 30 min. It was then concentrated to give the crude, which was slurried with celite and purified by reverse-phase column chromatography (0.1% FA in Water: ACN, 80 g Reveleris C18) and concentrated to get 4-(2-piperidylmethyl)phenol (4.5 g, 19.29 mmol, 44.01% yield) as off white solid. LCMS (ES⁺): m/z 192.33 [M+H]⁺.

### Step-5:

To a stirred solution of 4-(2-piperidylmethyl)phenol (4.5 g, 23.53 mmol) in methanol (5 mL), DCM (40 mL) was added triethylamine (11.90 g, 117.64 mmol, 16.40 mL) at 0° C. and was added di-tert-butyl dicarbonate (6.16 g, 28.23 mmol, 6.48 mL) stirred the reaction at rt for 16 h. After consumption of the starting material, the mixture was quenched with sat NaHCO₃ (50 mL), extracted with DCM (100 mL×2) and concentrated to give the crude, which was triturated with diethyl ether to afford tert-butyl 2-[(4-hydroxyphenyl)methyl]piperidine-1-carboxylate (3.2 g) as an off-white solid. LCMS (ES⁻): m/z 290.34 [M−H]⁻.

### Step-6:

To a stirred solution of tert-butyl 2-[(4-hydroxyphenyl)methyl]piperidine-1-carboxylate (5.7 g, 19.56 mmol) in DMF (30 mL), sodium hydride (60% dispersion in mineral oil) (1.12 g, 48.90 mmol) was added slowly at 0° C. and the reaction was stirred at room temperature for 30-40 min. Then the reaction mixture was cooled to 0° C. and 3-bromopiperidine-2,6-dione (5.63 g, 29.34 mmol) was added lot-wise slowly, and stirred at room temperature for 16 h. After completion of the reaction, the reaction mixture was quenched with ice cold sat. NH₄Cl solution and extracted with ethyl acetate (2×200 ml). The combined the organic layers were dried over sodium sulfate and concentrated under vacuum. The residue obtained was purified by column chromatography (Davisil-silica) using 40% EtOAc in pet ether as eluent to afford tert-butyl 2-[[4-[(2,6-dioxo-3-piperidyl)oxy]phenyl]methyl]piperidine-1-carboxylate (5.07 g, 12.25 mmol, 62.63% yield) as a white solid. LCMS (ES⁺): m/z 347.32 [M−^{t}Bu+H]⁺

### Step-7:

To a solution of tert-butyl 2-[[4-[(2,6-dioxo-3-piperidyl)oxy]phenyl]methyl]piperidine-1-carboxylate (600 mg, 1.49 mmol) in dioxane (8 mL) was added 4M HCl in dioxane (10 mL) at 0° C. and the reaction mixture was stirred at 25° C. for 2 h. The reaction mixture was concentrated in vacuo to give the crude product, which was triturated with diethyl ether (80 mL) to afford 3-[4-(2-piperidylmethyl)phenoxy]piperidine-2,6-dione (450 mg, 1.14 mmol, 76.19% yield, HCl salt) as an off-white solid. LCMS (ES⁻): m/z 303.26 [M+H]⁺.

### 3-(3-fluoro-4-(piperidin-4-yl)phenyl)piperidine-2,6-dione

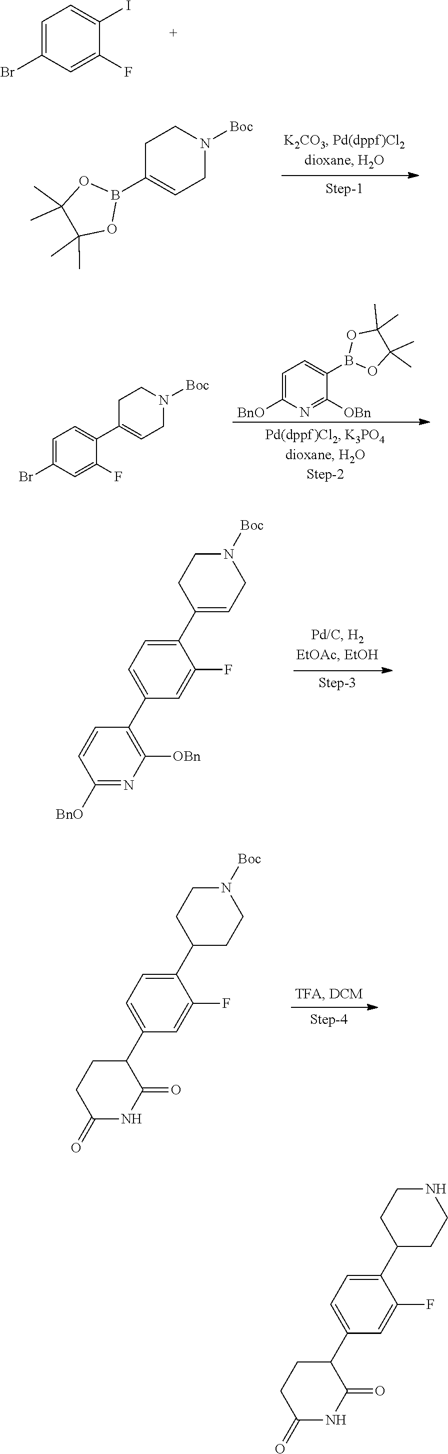

### Step-1:

To a solution of 4-bromo-2-fluoro-1-iodo-benzene (10.0 g, 33.23 mmol) and tert-butyl 4-(4,4,5,5-tetramethyl-1,3,2-dioxaborolan-2-yl)-3,6-dihydro-2H-pyridine-1-carboxylate (10.28 g, 33.23 mmol) in dioxane (918.58 μL) mL) and water (367.43 μL) was added potassium carbonate (13.78 g, 99.70 mmol) at RT. The reaction mixture was degassed with argon for 10 minutes and cyclopentyl(diphenyl)phosphane;dichloropalladium;iron (2.43 g, 3.32 mmol) was added. The reaction mixture was degassed with argon for an additional 5 minutes and stirred at 100° C. for 16 h. Subsequently, the reaction mixture was concentrated in vacuo to give the crude product, which was purified by column chromatography using Davisil silica and 7% ethyl acetate in pet ether as eluent to afford tert-butyl 4-(4-bromo-2-fluoro-phenyl)-3,6-dihydro-2H-pyridine-1-carboxylate (6.5 g, 18.25 mmol, 54.90% yield) as a colorless semi solid.

### Step-2:

To a solution of tert-butyl 4-(4-bromo-2-fluoro-phenyl)-3,6-dihydro-2H-pyridine-1-carboxylate (3.0 g, 8.42 mmol) and 2,6-dibenzyloxy-3-(4,4,5,5-tetramethyl-1,3,2-dioxaborolan-2-yl)pyridine (5.27 g, 12.63 mmol) in dioxane (24 mL) and water (6 mL) was added potassium phosphate tribasic anhydrous (5.36 g, 25.26 mmol) at RT. The reaction mixture was degassed with argon for 10 minutes and cyclopentyl(diphenyl)phosphane;dichloropalladium;iron (616.21 mg, 842.15 μmol) was added. The reaction mixture was degassed with argon for an additional 5 minutes and then stirred at 100° C. for 16 h. Subsequently, the reaction mixture was concentrated in vacuo to give the crude product, which was purified by column chromatography using Davisil silica and 5% ethyl acetate in pet ether as eluent to afford tert-butyl 4-[4-(2,6-dibenzyloxy-3-pyridyl)-2-fluoro-phenyl]-3,6-dihydro-2H-pyridine-1-carboxylate (2.25 g, 2.76 mmol, 32.72% yield) as a colorless semi solid. LCMS (ES⁺): m/z 567 [M+H]⁺.

### Step-3:

To a stirred solution of tert-butyl 4-[4-(2,6-dibenzyloxy-3-pyridyl)-2-fluoro-phenyl]-3,6-dihydro-2H-pyridine-1-carboxylate (2 g, 3.53 mmol) in ethanol (10 mL) and ethyl acetate (10 mL) was added 10% palladium on carbon (4 g) at room temperature. The reaction mixture was stirred under hydrogen atmosphere (balloon) for 16 h. Subsequently, the reaction mixture was filtered through celite bed and washed with ethyl acetate (30 mL). The filtrate was concentrated under reduced pressure to afford tert-butyl 4-[4-(2,6-dioxo-3-piperidyl)-2-fluoro-phenyl]piperidine-1-carboxylate (1.1 g, 2.08 mmol, 59.07% yield) as a brown liquid. LCMS (ES⁺): m/z 389 [M−H]⁻

### Step-4:

To a stirred solution of tert-butyl 4-[4-(2,6-dioxo-3-piperidyl)-2-fluoro-phenyl]piperidine-1-carboxylate (1 g, 2.56 mmol) in DCM (10 mL), 2,2,2-trifluoroacetic acid (2.96 g, 25.96 mmol, 2 mL) was added and stirred for 4 h at 0-25° C. After the reaction was complete, the reaction mixture was concentrated under reduced pressure to yield the crude compound, which was washed with diethyl ether to afford 3-[3-fluoro-4-(4-piperidyl)phenyl]piperidine-2,6-dione (0.9 g, 1.96 mmol, 76.48% yield, TFA salt) as a gray solid. LCMS (ES⁺): m/z 291 [M+H]⁺.

### Synthesis of 2-[1-[5-(2,6-dioxo-3-piperidyl)-3-fluoro-2-pyridyl]-4-piperidyl] acetic acid

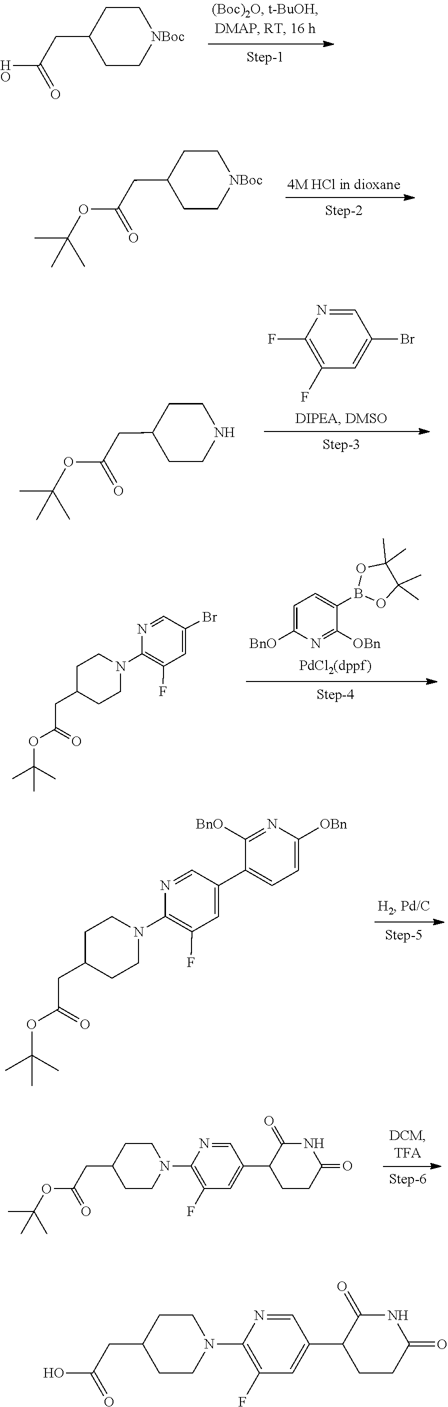

### Step-1:

To s stirred solution of 2-(1-tert-butoxycarbonyl-4-piperidyl) acetic acid (50 g, 205.51 mmol) in tert-butyl alcohol (500 mL) were added DMAP (2.51 g, 20.55 mmol) and di-tert-butyl dicarbonate (53.82 g, 246.61 mmol, 56.60 mL) dropwise at 0° C. under N₂ atmosphere over a period of 10 min. The resulting reaction mixture was stirred at room temperature for 16 h. After complete consumption of the starting material, excess tert-butyl alcohol was evaporated under vacuum, and the residue was diluted with water (200 mL) and extracted with EtOAc (2×500 mL). The combined organic layers were washed with water (200 mL), brine solution (300 mL), dried over anhydrous Na₂SO₄, and concentrated under reduced pressure. The crude compound was purified by column chromatography over silica gel (100-200 mesh) using 0-10% % of EtOAc in pet ether as eluent to afford tert-butyl 4-(2-tert-butoxy-2-oxo-ethyl)piperidine-1-carboxylate (35 g, 111.05 mmol, 54.04% yield) as an off white solid. ¹H NMR (400 MHz, DMSO-d₆): δ 3.89 (d, J=13.6 Hz, 2H), 2.67 (s, 2H), 2.12 (d, J=7.2 Hz, 2H), 1.77 (m, 1H), 1.55 (m, 2H), 1.38 (s, 18H), 0.99 (m, 2H).

### Step-2:

To a stirred solution of tert-butyl 4-(2-tert-butoxy-2-oxo-ethyl) piperidine-1-carboxylate (20 g, 66.80 mmol) in dioxane (725.25 mL) was added Hydrogen chloride solution 4.0 M in dioxane (83.50 mL) dropwise at 0° C. over a period of 15 min, the resulting mixture was stirred for 5 hours at 0° C. After the complete of starting material, excess dioxane was evaporated to obtain a solid compound, which was triturated with diethyl ether (50 mL) to afford tert-butyl 2-(4-piperidyl) acetate (12 g, 48.36 mmol, 72.39% yield, HCl salt). ¹H NMR (400 MHz, DMSO-d₆): δ 8.87 (s, 1H), 8.62 (s, 1H), 3.21 (m, 2H), 2.84 (m, 2H), 2.17 (m, 2H), 1.92 (m, 1H), 1.77 (m, 2H), 1.40 (m, 10H).

### Step-3:

To a stirred solution of tert-butyl 2-(4-piperidyl) acetate (3 g, 12.73 mmol, HCl salt) and 5-bromo-2,3-difluoro-pyridine (2.47 g, 12.73 mmol) in DMSO (13.15 mL) was added N, N-diisopropylethylamine (8.22 g, 63.63 mmol, 11.08 mL) dropwise at 120° C. and stirred for 4 hours. After complete consumption of starting material, the reaction mixture was quenched with ice cold water (30 mL) and extracted with ethyl acetate (2×100 mL). The combined organic layers were washed with brine (100 mL), dried over anhydrous sodium sulfate, filtered and concentrated under reduced pressure. The crude product was purified by column chromatography over silica gel (100-200 mesh) using 0-15% EtOAc in pet-ether as eluent to afford tert-butyl 2-[1-(5-bromo-3-fluoro-2-pyridyl)-4-piperidyl]acetate (3 g, 7.67 mmol, 60.31% yield). LCMS (ES⁺): m/z 373.55 [M+H]⁺.

### Step-4:

To a stirred solution of tert-butyl 2-[1-(5-bromo-3-fluoro-2-pyridyl)-4-piperidyl]acetate (3 g, 8.04 mmol) and 2,6-dibenzyloxy-3-(4,4,5-trimethyl-1,3,2-dioxaborolan-2-yl) pyridine (3.24 g, 8.04 mmol) in a mixture of water (5 mL) and dioxane (25 mL) was added cesium carbonate (7.86 g, 24.11 mmol). The resulting mixture was degassed with argon for 15 min before [1,1′-bis(diphenylphosphino)ferrocene] palladium(II) dichloride (589.53 mg, 802.96 μmol) was added to the reaction and heated at 80° C. for 16 h. After complete consumption of the starting material, the reaction mixture was filtered through celite bed, and the filtrate was diluted with water (20 mL) and extracted with ethyl acetate (2×50 mL). The combined organic layers were dried over sodium sulfate and concentrated under reduced pressure to give the crude, which was purified by column chromatography over silica gel (100-200 mesh) using 5 to 10% of ethyl acetate in hexane as eluent to afford tert-butyl 2-[1-[5-(2,6-dibenzyloxy-3-pyridyl)-3-fluoro-2-pyridyl]-4-piperidyl]acetate (2 g, 3.05 mmol, 37.94% yield) as yellow liquid. LCMS (ES⁺): m/z 584.4 [M+H]⁺.

### Step-5:

To a solution of tert-butyl 2-[1-[5-(2,6-dibenzyloxy-3-pyridyl)-3-fluoro-2-pyridyl]-4-piperidyl] acetate (2 g, 3.43 mmol) in ethyl acetate (20 mL) was added Palladium 10% on carbon (1.46 g, 13.71 mmol, 50% wet), the resultant mixture was hydrogenated with H₂ (in balloon pressure) and stirred vigorously at room temperatures for 16 h. After the complete consumption of starting material, the reaction mixture was filtered through celite bed, washed with ethyl acetate, the filtrate was concentrated and dried over high vacuum to get the crude compound which was purified by triturated with diethyl ether (10 mL) to afforded tert-butyl 2-[1-[5-(2,6-dioxo-3-piperidyl)-3-fluoro-2-pyridyl]-4-piperidyl] acetate 8 (800 mg, 1.89 mmol, 55.28% yield) as an off-white solid. LCMS (ES⁺): m/z 406.3 [M+H]⁺.

### Step-6:

A stirred solution of) tert-butyl 2-[1-[5-(2,6-dioxo-3-piperidyl)-3-fluoro-2-pyridyl]-4-piperidyl] acetate (0.300 g, 739.90 μmol) in DCM (1.62 mL) was cooled to 0° C. and TFA (843.65 mg, 7.40 mmol, 570.04 μL) was added over the period of 5 minutes followed by stirring at room temperature for 5 h. After consumption of the starting material, the reaction mixture was concentrated under reduced pressure and co-distilled with toluene (10 mL). The crude product was triturated with diethyl ether (2×10 mL) to afford 2-[1-[5-(2,6-dioxo-3-piperidyl)-3-fluoro-2-pyridyl]-4-piperidyl] acetic acid (0.250 g, 492.95 μmol, 66.62% yield, TFA salt) as an off-white solid. LCMS (ES⁺): m/z 350.50 [M+H]⁺.

### 2-[1-[5-(2,6-dioxo-3-piperidyl)-2-pyridyl]-4-piperidyl]acetic acid

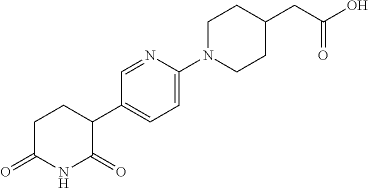 [SMILES: O=C(O)CC1CCN(c2ccc(C3CCC(=O)NC3=O)cn2)CC1]

This compound was prepared substantially following the synthesis of 2-[1-[5-(2,6-dioxo-3-piperidyl)-3-fluoro-2-pyridyl]-4-piperidyl] acetic acid, using 5-bromo-2-fluoro-pyridine in Step-3 instead of 5-bromo-2,3-difluoro-pyridine. LCMS (ES⁺): m/z 332.35 [M+H]⁺.

### Synthesis of 3-[6-(4-amino-1-piperidyl)-3-pyridyl]piperidine-2,6-dione

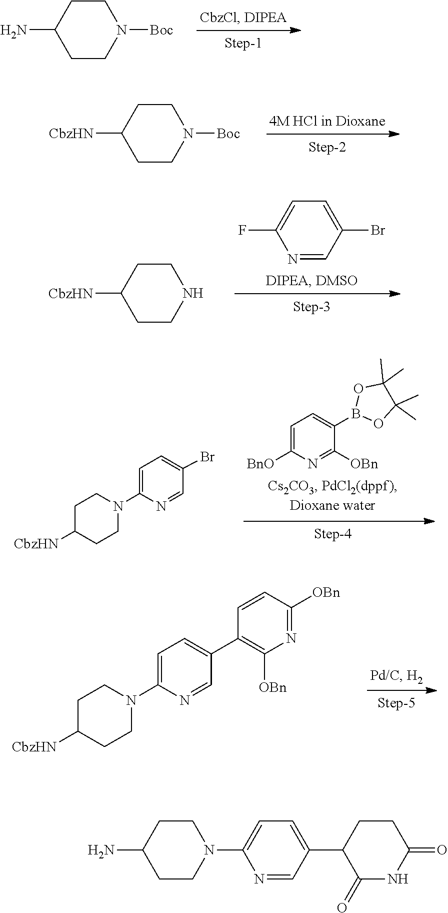

### Step-1:

To a stirred solution of tert-butyl 4-aminopiperidine-1-carboxylate (5 g, 24.97 mmol) in DCM (100 mL) was added DIPEA (9.68 g, 74.90 mmol, 13.05 mL) benzyl carbonochloridate (5.11 g, 29.96 mmol, 4.26 mL) at 0° C. The reaction mixture was stirred at 25° C. room temperature for 16 h. After complete consumption of starting material, the reaction mixture was quenched with sodium bicarbonate solution (50 mL) and extracted with DCM (2×250 mL). The combined organic layer was dried over anhydrous Na₂SO₄ and concentrated under reduced pressure. The crude product was purified by column chromatography using 100-200 mesh silica gel and 10-100% ethyl acetate in pet ether as eluent to afford tert-butyl 4-(benzyloxycarbonylamino)piperidine-1-carboxylate (8.5 g, 21.61 mmol, 86.54% yield). LCMS (ES⁺): m/z 235.19 [M+H]⁺.

### Step-2:

To a solution of tert-butyl 4-(benzyloxycarbonylamino)piperidine-1-carboxylate (8.5 g, 25.42 mmol) in 1,4-dioxane (80 mL) was added 4.0 M HCl in dioxane (85 mL) at 0° C. over the period of 5 minutes followed by stirring at room temperature for 2 h. After complete consumption of the starting material, the reaction mixture was concentrated under reduced pressure and co-distilled with toluene (10 mL) and diethyl ether (20×5 mL) to afford benzyl N-(4-piperidyl)carbamate 3 (5.5 g, 19.91 mmol, 78.32% yield) as an off white solid. LCMS (ES⁺): m/z 235.20 [M+H]⁺.

### Step-3:

To a stirred solution of benzyl N-(4-piperidyl)carbamate (3.38 g, 12.50 mmol, HCl salt) and 5-bromo-2-fluoro-pyridine (2 g, 11.36 mmol, 1.17 mL) in DMSO (15 mL) was added N,N-diisopropylethylamine (4.41 g, 34.09 mmol, 5.94 mL). The reaction was stirred at 80° C. for 16 h. After completion of the reaction, the reaction mixture was diluted by ice cold water (50 mL) and stirred for 20 min. The resulting solid was filtered under vacuum to afford benzyl N-[1-(5-bromo-2-pyridyl)-4-piperidyl]carbamate (3.3 g, 7.86 mmol, 69.20% yield) as a pale yellow solid. LCMS (ES⁺): m/z 390.52 [M+H]⁺.

### Step-4:

To a solution of N-[1-(5-bromo-2-pyridyl)-4-piperidyl]carbamate (1 g, 2.56 mmol) and 2,6-dibenzyloxy-3-(4,4,5,5-tetramethyl-1,3,2-dioxaborolan-2-yl)pyridine in a mixture of dioxane (7.5 mL) and water (2.5 mL) was added cesium carbonate (2.50 g, 7.69 mmol) at room temperature. The reaction mixture was degassed with argon for 10 minutes and added PdCl₂(dppf) DCM (313.87 mg, 384.35 μmol), the resulting reaction mixture was stirred at 100° C. for 16 h. After complete consumption of starting material, the reaction mixture was concentrated under reduced pressure. The crude product was purified by column chromatography (silica gel, 10-20% Ethyl acetate in Hexane) to afford benzyl N-[1-[5-(2,6-dibenzyloxy-3-pyridyl)-2-pyridyl]-4-piperidyl]carbamate (1 g, 1.38 mmol, 53.92% yield) as off-white solid. LCMS (ES⁺): m/z 601.69 [M+H]⁺.

### Step-5:

To stirred a solution of benzyl N-[1-[5-(2,6-dibenzyloxy-3-pyridyl)-2-pyridyl]-4-piperidyl]carbamate (1.25 g, 2.08 mmol) in the mixture of ethyl acetate (15 mL), THE (3 mL) and ethanol (2 mL) was added Palladium, 10% on carbon (2.5 g, 23.49 mmol) portionwise and the resulting mixture was stirred vigorously under hydrogen atmosphere (balloon) at room temperatures for 16 h. After complete consumption of starting material, the reaction mixture was filtered through celite bed, and washed with ethyl acetate. The filtrate was concentrated and dried under high vacuum. The crude product was purified by Prep-HPLC to afford 3-[6-(4-amino-1-piperidyl)-3-pyridyl]piperidine-2,6-dione (0.25 g, 0.86 mmol, 41% yield).

### Prep-HPLC Method:

Column: KINETEX C18 5 μm (21.2×250 mm)

Mobile Phase (A): 5 mM Ammonium Acetate in H₂O

Mobile Phase (B): 100% Acetonitrile

Flow Rate: 18 mL/min

Gradient (Time % B): 0/2, 3/2, 10/20, 12/20, 12.1/100, 16/100, 16.1/2, 18/2

LCMS (ES⁺): m/z 289.19 [M+H]⁺.

### 3-[6-(4-amino-1-piperidyl)-5-fluoro-3-pyridyl]piperidine-2,6-dione

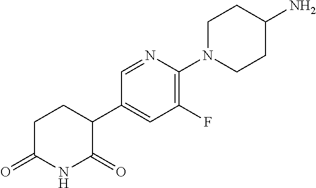 [SMILES: NC1CCN(c2ncc(C3CCC(=O)NC3=O)cc2F)CC1]

This compound was prepared substantially following the synthesis of 3-[6-(4-amino-1-piperidyl)-3-pyridyl]piperidine-2,6-dione, using 5-bromo-2,3-difluoro-pyridine in Step-3 instead of 5-bromo-2-fluoro-pyridine. LCMS (ES⁺): m/z 307.3 [M+H]⁺.

### Synthesis of 3-[6-[4-(methylamino)-1-piperidyl]-3-pyridyl] piperidine-2,6-dione

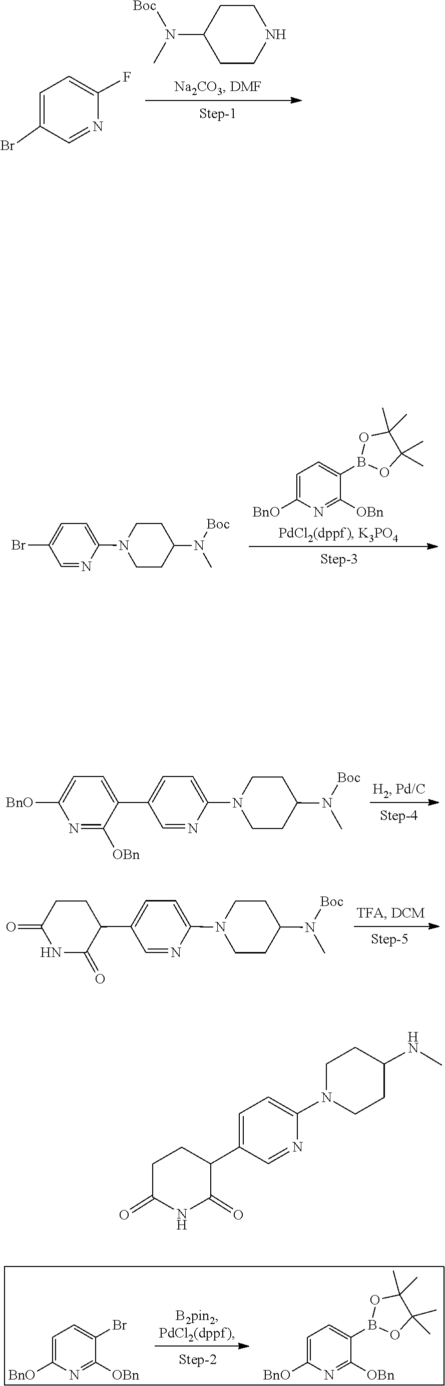

### Step-1:

A 10 mL microwave vial was charged with 5-bromo-2-fluoro-pyridine (2 g, 11.36 mmol, 1.17 mL), tert-butyl N-methyl-N-(4-piperidyl) carbamate (2.56 g, 11.93 mmol), sodium carbonate (3.01 g, 28.41 mmol) and DMF (30.60 mL). The reaction vial was sealed and the mixture was heated in a microwave reactor at 100° C. for 2 h. After complete consumption of starting material, the reaction mixture was cooled to ambient temperature and diluted with 30 mL of EtOAc. The mixture was then washed with water, brine, dried over anhydrous sodium sulfate, filtered and concentrated under reduced pressure. The resulting crude was purified by column chromatography using 100-200 silica gel and 0-50% ethyl acetate in pet ether as eluent to afford tert-butyl N-[1-(5-bromo-2-pyridyl)-4-piperidyl]-N-methyl-carbamate (3.2 g, 8.47 mmol, 74.52% yield) as a light yellow solid. LCMS (ES⁺): m/z 372.38 [M+H]⁺.

### Step-2:

To a stirred solution of 2,6-dibenzyloxy-3-bromo-pyridine (15 g, 40.51 mmol) in 1,4 dioxane (151.86 mL) were added 4,4,5,5-tetramethyl-2-(4,4,5,5-tetramethyl-1,3,2-dioxaborolan-2-yl)-1,3,2-dioxaborolane (15.43 g, 60.77 mmol) and potassium acetate (9.94 g, 101.29 mmol). The mixture was degassed with argon for 20 minute and was added cyclopentyl(diphenyl)phosphane dichloromethane dichloropalladium iron (3.31 g, 4.05 mmol) and heated to 100° C. for 16 h. After complete consumption of starting material, the reaction mixture was filtered through celite bed, washed with ethyl acetate (150 mL). The filtrate was concentrated under reduced pressure and the crude product was purified by column chromatography using silica gel (230-400 mesh) and 0-10% EtOAc in pet-ether as eluent to afford 2,6-dibenzyloxy-3-(4,4,5,5-tetramethyl-1,3,2-dioxaborolan-2-yl) pyridine (9 g, 16.61 mmol, 40.99% yield) as a pale-yellow liquid. LCMS (ES⁺): m/z 418.45 [M+H]⁺.

### Step-3:

To a stirred solution of tert-butyl N-[1-(5-bromo-2-pyridyl)-4-piperidyl]-N-methyl-carbamate 3 (0.5 g, 1.35 mmol) and 2,6-dibenzyloxy-3-(4,4,5,5-tetramethyl-1,3,2-dioxaborolan-2-yl) pyridine 5 (845.24 mg, 2.03 mmol) in a mixture of dioxane (5 mL) and water (1 mL) was added potassium phosphate tribasic anhydrous (859.88 mg, 4.05 mmol) at room temperature. The mixture was degassed with argon gas for 10 minutes and was added cyclopentyl(diphenyl)phosphane dichloropalladium iron (98.71 mg, 135.03 μmol). The reaction mixture was heated at 100° C. and stirred for 16 h. After complete consumption of starting material, the reaction mixture was filtered through celite bed, and washed with ethyl acetate (20 mL). The filtrate was concentrated under reduced pressure and the obtained crude product was purified by column chromatography using silica gel (230-400 mesh) and 10% ethyl acetate in pet ether as eluent to afford tert-butyl N-[1-[5-(2,6-dibenzyloxy-3-pyridyl)-2-pyridyl]-4-piperidyl]-N-methyl-carbamate (0.3 g, 413.28 μmol, 30.61% yield) as a white solid. LCMS (ES⁺): m/z 581.6 [M+H]⁺.

### Step-4:

To stirred a solution of tert-butyl N-[1-[5-(2,6-dibenzyloxy-3-pyridyl)-2-pyridyl]-4-piperidyl]-N-methyl-carbamate (0.3 g, 516.60 μmol) in the mixture of Ethyl acetate (20 mL), THF (5 mL) and ethanol (2 mL) was added 10% palladium on carbon (274.89 mg, 2.58 mmol) portionwise the resulting mixture was stirred vigorously under hydrogen atmosphere (balloon) at room temperatures for 16 h. After the complete consumption of starting material, the reaction mixture was filtered through celite bed, and washed with ethyl acetate and THF. The filtrate was concentrated and dried under high vacuum. The crude product was purified by column chromatography using silica gel (100-200 mesh) and 0-100% EtOAc in pet-ether as eluent to afford tert-butyl N-[1-[5-(2,6-dioxo-3-piperidyl)-2-pyridyl]-4-piperidyl]-N-methyl-carbamate (0.12 g, 283.72 μmol, 54.92% yield) as an off brown colour solid. LCMS (ES⁻): m/z 401.27 [M−H]⁻.

### Step-5:

To a solution of tert-butyl N-[1-[5-(2,6-dioxo-3-piperidyl)-2-pyridyl]-4-piperidyl]-N-methyl-carbamate (0.1 g, 248.46 μmol) in DCM (2 mL) was added TFA (95.71 μL, 1.24 mmol) at 0° C. over the period of 1 minute followed by stirring at room temperature for 5 h. After complete consumption of the starting material, the reaction mixture was concentrated under reduced pressure and co-distilled with toluene (10 mL) and diethyl ether (10 mL) to afford 3-[6-[4-(methylamino)-1-piperidyl]-3-pyridyl] piperidine-2,6-dione (70 mg, 199.25 μmol, 80.20% yield, formic acid salt) as a white solid. LCMS (ES⁺): m/z 303.16 [M+H]⁺.

### Synthesis of 2-[1-[5-(2,6-dioxo-3-piperidyl)-2-pyridyl]-4-piperidyl]acetic acid

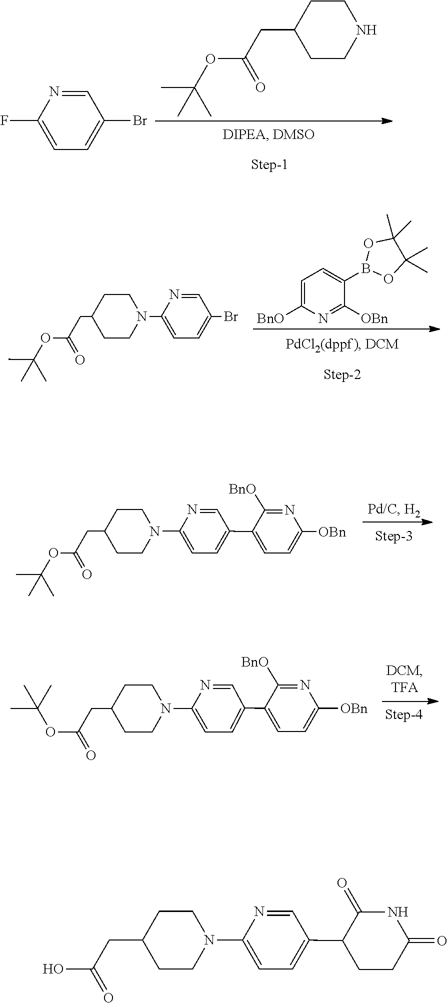

### Step-1:

To a stirred solution of tert-butyl 2-(4-piperidyl) acetate (5 g, 25.09 mmol) and 5-bromo-2-fluoro-pyridine (4.01 g, 22.81 mmol, 2.35 mL) in DMSO (40 mL) was added N, N-diisopropylethylamine (8.84 g, 68.43 mmol, 11.92 mL) dropwise over 10 min. The resulting mixture was heated to 120° C. and was stirred at this temperature for 4 hours. After the complete consumption of starting material, the reaction mixture was quenched with ice cold water (100 ml) and extracted with ethyl acetate (100 mL×4). The combined organic layers were washed with cold brine (200 mL) and dried over anhydrous sodium sulfate, filtered and concentrated under reduced pressure to afford tert-butyl 2-[1-(5-bromo-2-pyridyl)-4-piperidyl] acetate (5.5 g, 14.24 mmol, 62.45% yield). LCMS (ES⁺): m/z 355.3 [M+H]⁺

### Step-2:

To a stirred solution of 2,6-dibenzyloxy-3-(4,4,5,5-tetramethyl-1,3,2-dioxaborolan-2-yl) pyridine (7.05 g, 16.89 mmol) and tert-butyl 2-[1-(5-bromo-2-pyridyl)-4-piperidyl]acetate (5 g, 14.07 mmol) in a mixture of water (2 mL) and dioxane (15 mL) was added cesium carbonate (13.76 g, 42.22 mmol). The resulting mixture was degassed with argon for 10 minutes and Pd(dppf)Cl₂ (918.74 mg, 1.13 mmol) was added. The reaction mixture was heated to 100° C. and stirred for 16 h. After the complete consumption of starting material, the reaction mixture was filtered through a bed of celite. The filtrate was diluted with water (200 mL) and extracted with ethyl acetate (5×100 mL). The combined organic layers were dried over sodium sulfate, and concentrated in vacuo to give the crude product, which was purified by column chromatography (Davisil silica) using 25% ethyl acetate in hexane as eluent to afford methyl 2-[1-[5-(2,6-dibenzyloxy-3-pyridyl)-2-pyridyl]-4-piperidyl]acetate (2.4 g, 3.76 mmol, 26.71% yield) as an off-white solid. LCMS (ES⁺): m/z 566.6 [M+H]⁺

### Step-3:

To a stirred solution of tert-butyl 2-[1-[5-(2,6-dibenzyloxy-3-pyridyl)-2-pyridyl]-4-piperidyl] acetate (0.1 g, 176.77 μmol) in ethyl acetate (0.5 mL) was degassed with nitrogen for 10 min. Pd/C (0.1 g, 176.77 μmol) was added and the resulting mixture was stirred vigorously under hydrogen atmosphere (balloon) at room temperatures for 16 h. After the consumption of starting material, the reaction mixture was filtered through celite bed, and washed with EtOAc (50 mL). The filtrate was concentrated under reduced pressure to give the crude product, which was triturated with diethyl ether (10 mL) to afford tert-butyl 2-[1-[5-(2,6-dioxo-3-piperidyl)-2-pyridyl]-4-piperidyl] acetate (0.04 g, 102.20 μmol, 57.82% yield) as an off-white solid. LCMS (ES⁺): m/z 388.58 [M+H]⁺

### Step-4:

A solution of tert-butyl 2-[1-[5-(2,6-dioxo-3-piperidyl)-2-pyridyl]-4-piperidyl] acetate (0.030 g, 77.43 μmol) in DCM (998.13 μL) was cooled to 0° C. with stirring. Trifluoroacetic acid (8.83 mg, 77.43 μmol, 5.96 μL) was added dropwise over a period of 5 min followed by stirring at room temperature for 5 h. After complete consumption of the starting material, the reaction mixture was concentrated under vacuum to give the crude product, which was purified by prep-HPLC to afford 2-[1-[5-(2,6-dioxo-3-piperidyl)-2-pyridyl]-4-piperidyl]acetic acid (0.009 g, 23.36 μmol, 30.17% yield) as brown semi-solid. LCMS (ES⁺): m/z 332.5 [M+H]⁺.

### 2-[1-[5-(2,6-dioxo-3-piperidyl)-3-fluoro-2-pyridyl]-4-hydroxy-4-piperidyl] acetic acid

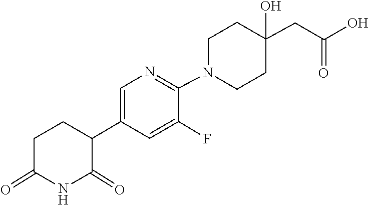 [SMILES: O=C(O)CC1(O)CCN(c2ncc(C3CCC(=O)NC3=O)cc2F)CC1]

This compound was prepared substantially following the synthesis of 2-[1-[5-(2,6-dioxo-3-piperidyl)-2-pyridyl]-4-piperidyl]acetic acid, using tert-butyl 2-(4-hydroxy-4-piperidyl) acetate and 5-bromo-2,3-difluoro-pyridine as starting materials. LCMS (ES⁺): m/z 366.11 [M+H]⁺.

### Synthesis of 3-[6-[4-(aminomethyl)-4-hydroxy-1-piperidyl]-5-fluoro-3-pyridyl] piperidine-2,6-dione

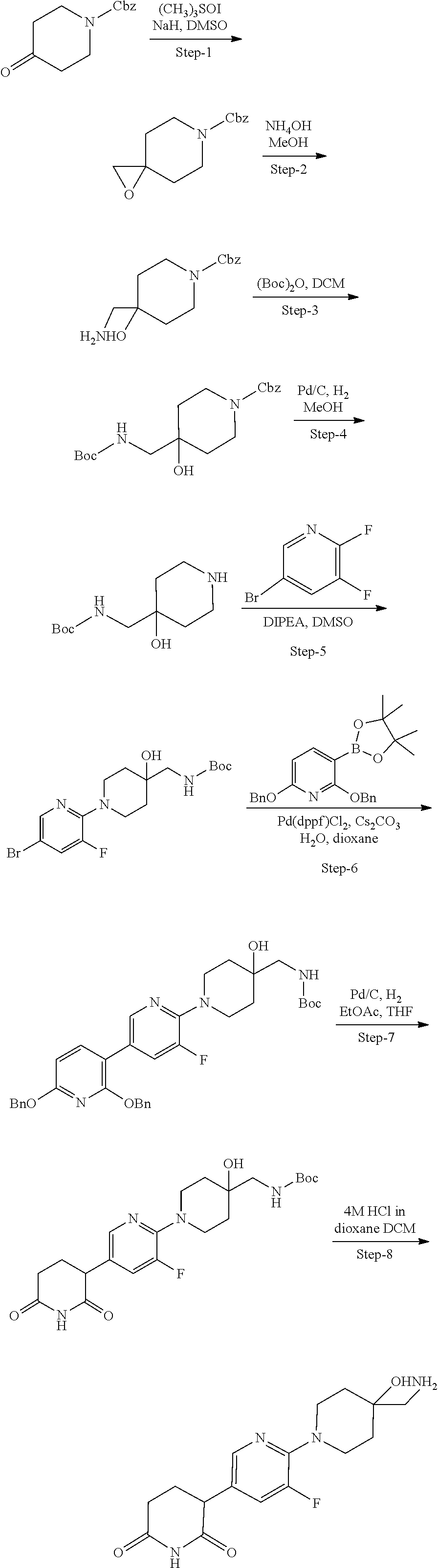

### Step-1:

A solution of sodium hydride (60% dispersion in mineral oil) (6.41 g, 278.66 mmol, 4.64 mL)and trimethylsulfoxonium iodide, 98+% (51.89 g, 235.79 mmol) in DMSO (5 mL) was stirred at 10° C. for 10 min. After completion of the reaction, the reaction mixture was diluted with water and extracted with ethyl acetate. The organic layer dried over sodium sulfate and concentrated to afford benzyl 1-oxa-6-azaspiro[2.5]octane-6-carboxylate (50 g, 74.81 mmol, 34.90% yield) as a yellow liquid. LCMS (ES⁺). m/z 248.17 [M+H]⁺

### Step-2:

A solution of benzyl 1-oxa-6-azaspiro[2.5]octane-6-carboxylate (51 g, 206.24 mmol)and ammonium hydroxide, 28% solution (550.80 g, 15.71 mol, 612.00 mL) in DMSO (5 mL) was stirred at 10° C. for 10 min. After completion of the reaction, the reaction mixture was diluted with water and extracted with ethyl acetate. The organic layer was dried over sodium sulfate and concentrated to afford benzyl 4-(aminomethyl)-4-hydroxy-piperidine-1-carboxylate (51 g, 94.54 mmol, 45.84% yield) as a yellow liquid. LCMS (ES⁺): m/z 265.59 [M+H]⁺

### Step-3:

A solution of benzyl 4-(aminomethyl)-4-hydroxy-piperidine-1-carboxylate (60 g, 227.00 mmol) and di-tert-butyl dicarbonate (49.54 g, 227.00 mmol, 52.09 mL) in DCM (77.9 mL) was stirred at 10° C. for 10 min. After completion of the reaction, the reaction mixture was diluted with water (300 ml) and extracted with ethyl acetate (4×200). The organic layer was dried over sodium sulfate and concentrated to afford benzyl 4-[(tert-butoxycarbonylamino)methyl]-4-hydroxy-piperidine-1-carboxylate (60 g, 74.09 mmol, 32.64% yield) as a yellow liquid. LCMS (ES⁺): m/z 265.2 [M+H]⁺

### Step-4:

To a stirred solution of benzyl 4-[(tert-butoxycarbonylamino)methyl]-4-hydroxy-piperidine-1-carboxylate (60 g, 164.64 mmol) in methanol (10 mL) was added Palladium, 10% on carbon, Type 487, dry (17.52 g, 164.64 mmol) and stirred at RT under hydrogen atmosphere for 16 h. Upon completion of the reaction, the reaction mixture was filtered through celite, washed with MeOH. The filtrate was evaporated under reduced pressure to get crude tert-butyl N-[(4-hydroxy-4-piperidyl)methyl]carbamate (31 g, 123.84 mmol, 75.22% yield) as yellow liquid. LCMS (ES⁺): m/z 231.25 [M+H]⁺

### Step-5:

To a stirred solution of tert-butyl N-[(4-hydroxy-4-piperidyl)methyl]carbamate (6.53 g, 28.35 mmol) and 5-bromo-2,3-difluoro-pyridine (5 g, 25.78 mmol) in DMSO (47.88 mL) was added N,N-Diisopropylethylamine (9.99 g, 77.33 mmol, 13.47 mL) dropwise. The reaction was allowed to stir at 120° C. for 4 hours. After completion of the reaction, it was quenched with ice cold water (100 ml) and extracted with ethyl acetate (100×4 ml). The combined organic layer was washed with cold brine (200 ml) and dried over anhydrous sodium sulfate, filtered and concentrated under reduced pressure to afford tert-butyl N-[[1-(5-bromo-3-fluoro-2-pyridyl)-4-hydroxy-4-piperidyl]methyl]carbamate (5.8 g, 13.49 mmol, 52.32% yield). LCMS (ES⁺): m/z 203.0 [M+H]⁺

### Step-6:

To a solution of tert-butyl N-[[1-(5-bromo-3-fluoro-2-pyridyl)-4-hydroxy-4-piperidyl]methyl]carbamate (5 g, 12.37 mmol) and 2,6-dibenzyloxy-3-(4,4,5,5-tetramethyl-1,3,2-dioxaborolan-2-yl)pyridine (7.74 g, 18.55 mmol) in water (2 mL) and dioxane (10 mL) was added cesium carbonate (12.09 g, 37.10 mmol) at RT. The reaction mixture was degassed with argon for 10 minutes and Pd(dppf)Cl₂ DCM (807.37 mg, 989.43 μmol) was added. The reaction mixture was degassed with argon for an additional 5 minutes and stirred at 100° C. for 16 h. After completion of the reaction, the reaction mixture was filtered through celite. The filtrate was diluted with water (200 mL) and extracted with ethyl acetate (5×100 mL). The organic layer was dried over sodium sulfate and concentrated in vacuo to give the crude product, which was purified by column chromatography using Davisil silica and 25% ethyl acetate in hexane as eluent to afford tert-butyl N-[[1-[5-(2,6-dibenzyloxy-3-pyridyl)-3-fluoro-2-pyridyl]-4-hydroxy-4-piperidyl]methyl]carbamate (4 g, 4.95 mmol, 39.99% yield) as a yellow liquid. LCMS (ES⁺): m/z 615.4 [M+H]⁺

### Step-7:

A stirred solution of tert-butyl N-[[1-[5-(2,6-dibenzyloxy-3-pyridyl)-3-fluoro-2-pyridyl]-4-hydroxy-4-piperidyl]methyl]carbamate (2 g, 3.25 mmol) in EtOAc (10 mL) was degassed with nitrogen for 10 min. 10% Palladium on carbon (346.25 mg, 3.25 mmol) was added at 25° C. and the reaction was stirred at this temperature for 16 h under hydrogen atmosphere. Upon completion of the reaction, the reaction mixture was filtered through celite bed, and washed with THF: EtOAc (200 mL). The filtrate was concentrated under reduced pressure to give the crude, which was washed with diethyl ether to afford tert-butyl N-[[1-[5-(2,6-dioxo-3-piperidyl)-3-fluoro-2-pyridyl]-4-hydroxy-4-piperidyl]methyl]carbamate (0.750 g, 1.62 mmol, 49.64% yield) as a blue solid. LCMS (ES⁺): m/z 437.4 [M+H]⁺

### Step-8:

To a stirred solution of tert-butyl N-[[1-[5-(2,6-dioxo-3-piperidyl)-3-fluoro-2-pyridyl]-4-hydroxy-4-piperidyl]methyl]carbamate (0.8 g, 1.83 mmol) in DCM (8 mL) was added 4.0M hydrogen chloride solution in dioxane (8 mL) at 0° C. and the reaction mixture was stirred at room temperature for 1 h. After completion of the reaction, the reaction mixture was concentrated under reduced pressure to give the crude product, which was triturated with diethyl ether (50 mL) and dried under reduced pressure to afford 3-[6-[4-(aminomethyl)-4-hydroxy-1-piperidyl]-5-fluoro-3-pyridyl]piperidine-2,6-dione (0.6 g, 1.51 mmol, 82.54% yield, HCl salt) as an off white solid. LCMS (ES⁺): m/z 337.35 [M+H]⁺

### Synthesis of 3-(6-piperazin-1-yl-3-pyridyl)piperidine-2,6-dione

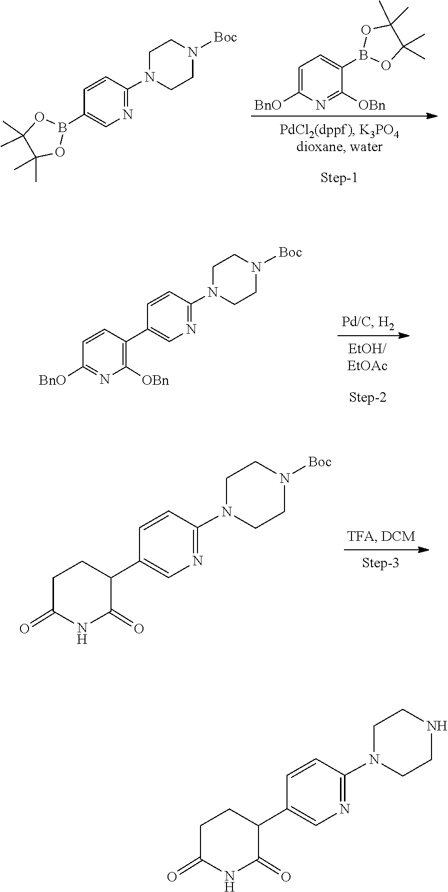 [SMILES: CC(C)(C)OC(=O)N1CCN(c2ccc(B3OC(C)(C)C(C)(C)O3)cn2)CC1 ; CC1(C)OB(c2ccc(OCc3ccccc3)nc2OCc2ccccc2)OC1(C)C ; CC(C)(C)OC(=O)N1CCN(c2ccc(-c3ccc(OCc4ccccc4)nc3OCc3ccccc3)cn2)CC1 ; CC(C)(C)OC(=O)N1CCN(c2ccc(C3CCC(=O)NC3=O)cn2)CC1 ; O=C1CCC(c2ccc(N3CCNCC3)nc2)C(=O)N1]

### Step-1:

To a stirred solution of tert-butyl 4-[5-(4,4,5,5-tetramethyl-1,3,2-dioxaborolan-2-yl)-2-pyridyl]piperazine-1-carboxylate (CAS #496786-98-2) (5.3 g, 13.61 mmol,) and 2,6-dibenzyloxy-3-bromo-pyridine (4.20 g, 11.35 mmol) in 1,4-dioxane (100 mL) and water (25 mL) was added dipotassium carbonate (3.14 g, 22.69 mmol) and purged with nitrogen gas for 15 min. Then cyclopentyl(diphenyl)phosphane;dichloropalladium;iron (415.07 mg, 567.26 μmol) was added and purged with nitrogen gas for another 5 min. The reaction mixture was heated to 90° C. for 16 hours. After completion of the reaction, the reaction mixture was filtered through a bed of celite and the filtrate was concentrated in vacuo. The crude material was purified by column chromatography (230-400 mesh silica gel, 15% ethyl acetate/pet ether as eluent) to give tert-butyl 4-[5-(2,6-dibenzyloxy-3-pyridyl)-2-pyridyl]piperazine-1-carboxylate (4.5 g, 6.92 mmol, 61.00% yield) as off white solid. LC-MS (ES⁺): m/z 553.84 [M+H]⁺. ¹H NMR (400 MHz, CDCl₃): δ 8.37 (d, J=2.4 Hz, 1H), 7.75 (dd, J=8.8 Hz, J=2.4 Hz, 1H), 7.55 (d, J=8.4 Hz, 1H), 7.43-7.27 (m, 10H), 6.66 (d, J=8.8 Hz, 1H), 6.46 (d, J=8.4 Hz, 1H), 5.42 (s, 2H), 5.35 (s, 2H), 3.53 (bs, 8H), 1.49 (s, 9H).

### Step-2:

To a stirred solution of tert-butyl 4-[5-(2,6-dibenzyloxy-3-pyridyl)-2-pyridyl]piperazine-1-carboxylate (4.5 g, 8.14 mmol) in ethyl acetate (100 mL) and ethanol (100 mL) was added palladium on carbon (4.50 g, 42.29 mmol). The reaction mixture was stirred under hydrogen gas (balloon) at room temperature for 12 hours. After completion of reaction, the reaction mixture was filtered through a bed of celite, concentrated, and purified by column chromatography using 230-400 mesh silica gel and 95% ethyl acetate in pet ether to afford tert-butyl 4-[5-(2,6-dioxo-3-piperidyl)-2-pyridyl]piperazine-1-carboxylate (2.1 g, 5.33 mmol, 65.44% yield). LC-MS (ES⁺): m/z 375.45 [M+H]⁺.

### Step-3:

To a stirred solution of tert-butyl 4-[5-(2,6-dioxo-3-piperidyl)-2-pyridyl]piperazine-1-carboxylate (1.5 g, 4.01 mmol) in DCM (20 mL) under an inert atmosphere was added 2,2,2-trifluoroacetic acid (22.20 g, 194.70 mmol, 15 mL) at 0° C. Then the reaction mixture was stirred at room temperature for 1 hr. After completion of the reaction, the mixture was concentrated under reduced pressure and triturated with diethyl ether (2×100 mL), and dried to obtain 3-(6-piperazin-1-yl-3-pyridyl)piperidine-2,6-dione (1.5 g, 3.79 mmol, 94.49% yield, TFA salt) as an off-white solid. LC-MS (ES⁺): m/z 275. [M+H]⁺.

### Synthesis of 2-[4-[5-(2,6-dioxo-3-piperidyl)-2-pyridyl]piperazin-1-yl]acetic

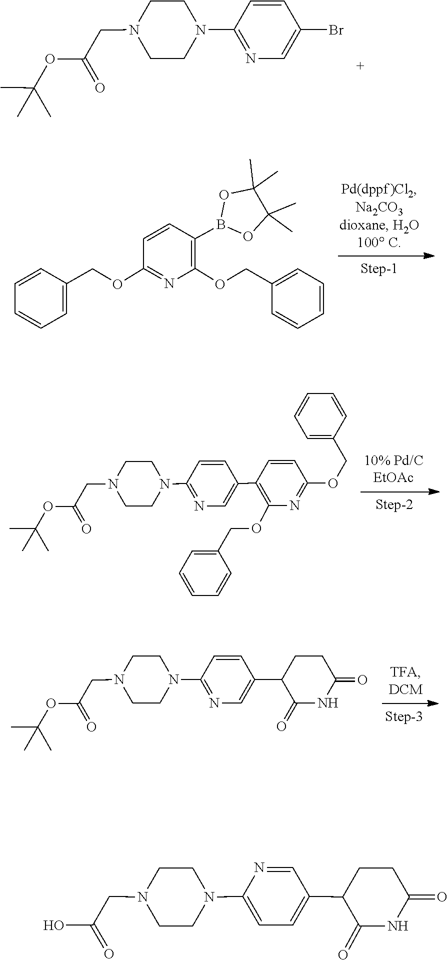 [SMILES: CC(C)(C)OC(=O)CN1CCN(c2ccc(Br)cn2)CC1 ; CC1(C)OB(c2ccc(OCc3ccccc3)nc2OCc2ccccc2)OC1(C)C ; CC(C)(C)OC(=O)CN1CCN(c2ccc(-c3ccc(OCc4ccccc4)nc3OCc3ccccc3)cn2)CC1 ; CC(C)(C)OC(=O)CN1CCN(c2ccc(C3CCC(=O)NC3=O)cn2)CC1 ; O=C(O)CN1CCN(c2ccc(C3CCC(=O)NC3=O)cn2)CC1]

### Step-1:

To a stirred solution of tert-butyl 2-[4-(5-bromo-2-pyridyl)piperazin-1-yl]acetate (3.0 g, 8.42 mmol, WO9322303) and 2,6-dibenzyloxy-3-(4,4,5,5-tetramethyl-1,3,2-dioxaborolan-2-yl)pyridine (5.27 g, 12.63 mmol) in 1,4-dioxane (40 mL) and water (10 mL) was added sodium carbonate (2.68 g, 25.26 mmol, 1.06 mL) and thoroughly purged with argon. Cyclopentyl(diphenyl)phosphane;dichloromethane;dichloropalladium;iron (687.68 mg, 842.09 umol) was added under inert an atmosphere. The resulting mixture was heated at 100° C. for 16 h. The reaction mixture was diluted with ethyl acetate, filtered through a short pad of celite and washed with ethyl acetate. Combined organic layer was washed with water, brine, dried over anhydrous sodium sulphate, filtered and concentrated under reduced pressure. Purification by column chromatography (15-20% ethyl acetate-hexane) gave tert-butyl 2-[4-[5-(2,6-dibenzyloxy-3-pyridyl)-2-pyridyl]piperazin-1-yl]acetate (3.5 g, 6.11 mmol, 72.51% yield). LC-MS (ES⁺): m/z 567.0 [M+H]⁺.

### Step-2:

Tert-butyl 2-[4-[5-(2,6-dibenzyloxy-3-pyridyl)-2-pyridyl]piperazin-1-yl]acetate (3.5 g, 6.18 mmol) was dissolved in ethyl acetate (35 mL) and the solution was degassed with nitrogen gas for 15 minutes. 10% Palladium on carbon wet (3 g, 28.19 mmol) was added and the reaction mixture was stirred under hydrogen atmosphere (hydrogen balloon) for 20 hours. The reaction mixture was filtered through celite, celite bed was washed with ethyl acetate and filtrate was concentrated under reduced pressure. Purification by silica gel chromatography (65%-70% ethyl acetate-hexane using 230-400 mesh) gave tert-butyl 2-[4-[5-(2,6-dioxo-3-piperidyl)-2-pyridyl]piperazin-1-yl]acetate (1.2 g, 3.02 mmol, 48.96% yield, 97.88% purity) as reddish white solid. ¹H NMR (400 MHz, DMSO-d₆): δ 10.80 (bs, 1H), 7.95 (bs, 1H), 7.39 (d, J=8.6 Hz, 1H), 6.79 (d, J=8.6 Hz, 1H), 3.76-3.71 (m, 1H), 3.47-3.42 (m, 4H), 3.15 (s, 2H), 2.71-2.62 (m, 1H), 2.60-2.50 (m, 5H), 2.21-2.13 (m, 1H), 1.98-1.96 (m, 1H), 1.41 (s, 9H).

### Step-3:

To a stirred solution of tert-butyl 2-[4-[5-(2,6-dioxo-3-piperidyl)-2-pyridyl]piperazin-1-yl]acetate (500 mg, 1.29 mmol) in DCM (20 mL) was added TFA (146.76 mg, 1.29 mmol, 99.16 μL) at 0° C. The reaction mixture was then stirred at RT for 16 h. After consumption of the starting material, the solvent was removed to give a residue, which was triturated with diethyl ether to yield a solid precipitate. The diethyl ether layer was decanted and the solid was dried under vacuum to afford 2-[4-[5-(2,6-dioxo-3-piperidyl)-2-pyridyl]piperazin-1-yl]acetic acid (500 mg, 940.91 μmol, 73.10% yield, TFA salt) as a brown solid. LCMS (ES⁺): m/z 333.29 [M+H]⁺

### Synthesis of 3-[6-(2,7-diazaspiro[3.5] nonan-7-yl)-5-fluoro-3-pyridyl]piperidine-2,6-dione

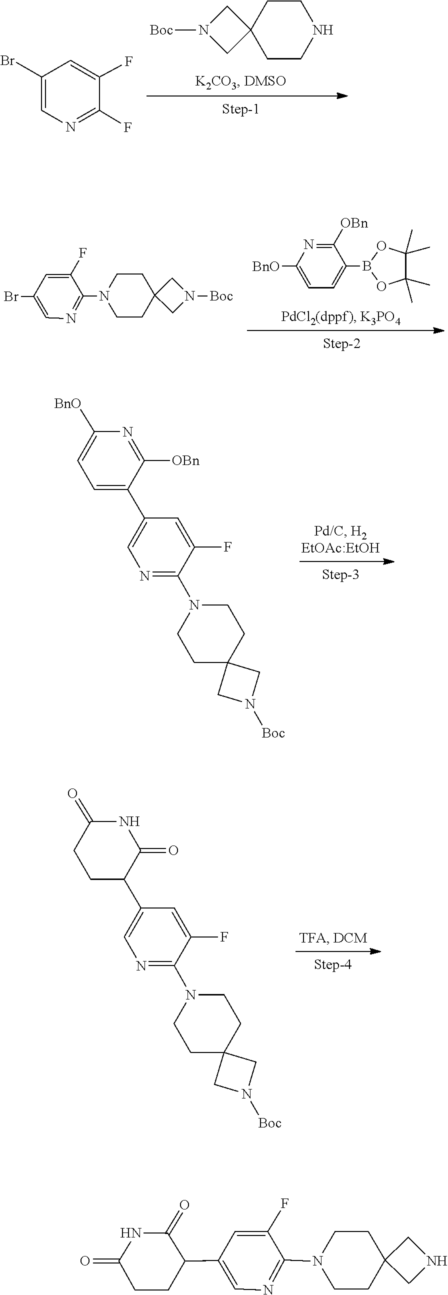

### Step-1:

A round bottom flask was charged with 5-bromo-2,3-difluoro-pyridine (1.10 g, 5.68 mmol), tert-butyl 2,7-diazaspiro[3.5]nonane-2-carboxylate (1.17 g, 5.17 mmol), K₂CO₃ (2.14 g, 15.50 mmol) and DMSO (10 mL). The reaction mixture was heated at 60° C. for 16 h. After complete consumption of starting material, the crude reaction mixture was cooled to ambient temperature and diluted with 30 mL of EtOAc. The organic layer was washed with water, brine, dried over anhydrous sodium sulfate, filtered and concentrated under reduced pressure give the crude product, which was purified by column chromatography using 100-200 silica gel and 0-50% ethyl acetate in pet ether to afford tert-butyl 7-(5-bromo-3-fluoro-2-pyridyl)-2,7-diazaspiro[3.5]nonane-2-carboxylate (1.0 g, 2.45 mmol, 47.39% yield) as an off white solid. LCMS (ES⁺): m/z 401.72 [M+H]⁺

### Step-2:

To a stirred solution of tert-butyl 7-(5-bromo-3-fluoro-2-pyridyl)-2,7-diazaspiro[3.5]nonane-2-carboxylate (500 mg, 1.25 mmol) and 2,6-dibenzyloxy-3-(4,4,5,5-tetramethyl-1,3,2-dioxaborolan-2-yl) pyridine (4.1 g, 10.04 mmol) in a mixture of dioxane (2 mL) and water (1 mL) was added tripotassium carbonate (361.52 mg, 2.62 mmol) at room temperature. The resulting mixture was degassed with argon for 10 minutes and added cyclopentyl(diphenyl)phosphane dichloropalladium iron (91.40 mg, 124.91 μmol). The reaction was then heated at 90° C. with stirring for 16 h. After complete consumption of starting material, the reaction mixture was filtered through a bed of celite and washed with ethyl acetate (20 mL). The filtrate was concentrated under reduced pressure to give the crude product, which was purified by column chromatography using silica gel (230-400 mesh) and 10% ethyl acetate in pet ether as eluent to afford tert-butyl 7-[5-(2,6-dibenzyloxy-3-pyridyl)-3-fluoro-2-pyridyl]-2,7-diazaspiro[3.5]nonane-2-carboxylate (200 mg, 311.11 μmol, 24.91% yield) as an off white solid. LCMS (ES⁺): m/z 611.52 [M+H]⁺

### Step-3:

To a stirred solution of tert-butyl 7-[5-(2,6-dibenzyloxy-3-pyridyl)-3-fluoro-2-pyridyl]-2,7-diazaspiro[3.5]nonane-2-carboxylate (1 g, 1.64 mmol) in ethyl acetate (18 mL) and ethanol (2 mL) was added Palladium, 10% on carbon (2.31 g, 9.82 mmol) portionwise and the resulting mixture was stirred vigorously under hydrogen atmosphere (balloon) at room temperatures for 16 h. After complete consumption of the starting material, the reaction mixture was filtered through a bed of celite, and washed with ethyl acetate and THF. The filtrate was concentrated and co-distilled with toluene (10 mL) and then triturated with diethyl ether (10 mL) to afford tert-butyl 7-[5-(2,6-dioxo-3-piperidyl)-3-fluoro-2-pyridyl]-2,7-diazaspiro[3.5]nonane-2-carboxylate (600 mg, 1.37 mmol, 83.73% yield) as a grey solid. LCMS (ES⁻): m/z 431.51 [M−H]⁻

### Step-4:

A solution of tert-butyl 7-[5-(2,6-dioxo-3-piperidyl)-3-fluoro-2-pyridyl]-2,7-diazaspiro[3.5]nonane-2-carboxylate (1 g, 2.31 mmol) in DCM (10 mL) was cooled to 0° C. and added TFA (2.64 g, 23.12 mmol, 1.78 mL) over the period of 5 minutes. The reaction was stirred at room temperature for 2 h. After complete consumption of the starting material, the reaction mixture was concentrated under reduced pressure and co-distilled with toluene (10 mL) and then triturated with diethyl ether (10 mL) to afford 3-[6-(2,7-diazaspiro[3.5]nonan-7-yl)-5-fluoro-3-pyridyl]piperidine-2,6-dione (1 g, 2.21 mmol, 95.66% yield, TFA salt) as an off white solid. LCMS (ES⁺): m/z 333.20 [M+H]⁺

### 3-[6-(2,6-diazaspiro[[3.3]heptan-2-yl)-5-fluoro-3-pyridyl]piperidine-2,6-dione

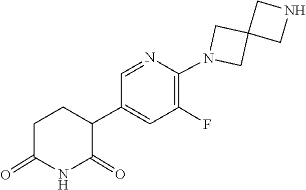 [SMILES: O=C1CCC(c2cnc(N3CC4(CNC4)C3)c(F)c2)C(=O)N1]

This compound was prepared substantially following the synthesis of 3-[6-(2,7-diazaspiro[3.5] nonan-7-yl)-5-fluoro-3-pyridyl]piperidine-2,6-dione, using tert-butyl 2,6-diazaspiro[3.3]heptane-2-carboxylate instead of tert-butyl 2,7-diazaspiro[3.5]nonane-2-carboxylate in Step-1. LCMS (ES⁺). m/z 305.47 [M+H]⁺.

### Synthesis of 2-[2-[5-(2,6-dioxo-3-piperidyl)-2-pyridyl]-2-azaspiro[3.3]heptan-6-yl]acetic acid

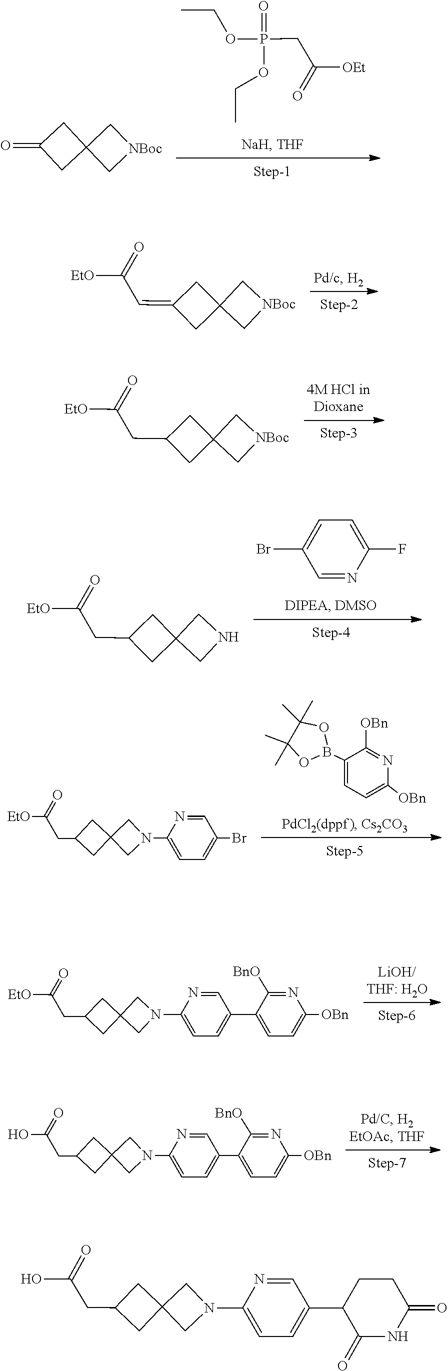

### Step-1:

To a stirred solution of ethyl 2-diethoxyphosphorylacetate (7.96 g, 35.50 mmol, 7.07 mL) was added Sodium hydride (in oil dispersion) 60% dispersion in mineral oil (2.36 g, 59.17 mmol) in THF (50 mL) was cooled to 0° C. tert-butyl 6-oxo-2-azaspiro [3.3] heptane-2-carboxylate (5 g, 23.67 mmol) was added. The reaction mixture was allowed to stir at room temperature for 2 h. After complete consumption of starting material, the reaction mixture was quenched with saturated aqueous solution of NaCl (50 mL) and extracted with EtOAc (50 mL×2). The combined organic layers were dried over anhydrous sodium sulfate, filtered and concentrated in vacuo to give the crude product, which was purified by column chromatography (100-200 mesh silica gel, 10-20% ethyl acetate in Pet ether as eluent) to afford tert-butyl-6-(2-ethoxy-2-oxo-ethylidene)-2-azaspiro [3.3] heptane-2-carboxylate (1.5 g, 5.27 mmol, 22.27% yield). LCMS (ES⁺): m/z 282.36 [M+H]⁺.

### Step-2:

In a 100 mL round bottom flask, to the solution of tert-butyl 6-(2-ethoxy-2-oxo-ethylidene)-2-azaspiro [3.3] heptane-2-carboxylate (4.5 g, 15.99 mmol) in ethanol (50 mL) was added Palladium, 10% on carbon, Type 487, dry (2.87 g, 26.99 mmol) and the reaction was stirred under hydrogen atmosphere (balloon) at room temperature for 16 h. After complete consumption of starting material, the reaction mixture was filtered through a pad of celite and washed with ethyl acetate (2×100 mL). The filtrate was concentrated under reduced pressure to afford tert-butyl 6-(2-ethoxy-2-oxo-ethyl)-2-azaspiro [3.3] heptane-2-carboxylate (3.5 g, 12.35 mmol, 77.22% yield) as a gum.

### Step-3:

To the stirred solution of tert-butyl 6-(2-ethoxy-2-oxo-ethyl)-2-azaspiro [3.3] heptane-2-carboxylate (3.5 g, 12.35 mmol) in DCM (20 mL) was added 4.0 M hydrogen chloride solution in dioxane (16.00 g, 438.84 mmol, 20.00 mL) at room temperature and the reaction was stirred for 2 h. After complete consumption of starting material, the solvent was removed by under reduced pressure to give the crude product, which was triturated with diethyl ether (20 mL) to afford ethyl 2-(2-azaspiro [3.3] heptan-6-yl) acetate (2.5 g, 11.38 mmol, 92.12% yield) as a gum.

### Step-4:

To the stirred solution of 5-bromo-2-fluoro-pyridine (2.20 g, 12.52 mmol, 1.29 mL), N, N-diisopropylethylamine (7.35 g, 56.89 mmol, 9.91 mL) in DMSO (10 mL) was added ethyl 2-(2-azaspiro [3.3] heptan-6-yl) acetate (2.5 g, 11.38 mmol) and the resulting reaction mixture was heated at 100° C. for 16 h. After complete consumption of stating material, the reaction mixture was quenched with ice cold water (20 mL) and extracted with EtOAc (2×100 mL). The combined organic layers were dried over anhydrous Na₂SO₄ and concentrated under reduced pressure to give the crude product, which was purified by column chromatography (100-200 mesh SiO₂, 10-20% EtOAc in Pet ether as eluent) to afford tert-butyl ethyl 2-[2-(5-bromo-2-pyridyl)-2-azaspiro[3.3]heptan-6-yl]acetate (2 g, 5.20 mmol, 45.70% yield) as an off-white gummy solid. LCMS (ES⁺): m/z 340.62 [M+H]⁺.

### Step-5:

To a stirred solution of ethyl 2-[2-(5-bromo-2-pyridyl)-2-azaspiro [3.3] heptan-6-yl]acetate (2 g, 5.90 mmol) in a mixture of Dioxane (16 mL) and Water (4 mL) in a sealed tube was added 2,6-dibenzyloxy-3-(4,4,5,5-tetramethyl-1,3,2-dioxaborolan-2-yl) pyridine (3.20 g, 7.66 mmol) and Cesium carbonate (5.76 g, 17.69 mmol). The reaction mixture was degassed with argon for 10 minutes. [1,1′-Bis(diphenylphosphino)ferrocene]dichloropalladium(II) complex (259 mg, 354 μmol) was added and the resulting mixture was heated to 100° C. and stirred for 6 h. After complete consumption of starting material, the reaction was then cooled to room temperature and filtered through a short bed of celite. The filtrate was diluted with ethyl acetate (100 mL), washed with water, dried over anhydrous Na₂SO₄ and concentrated under reduced pressure to give the crude product, which was purified by column chromatography (230-400 mesh silica gel, 10-20% ethyl acetate in hexane as eluent) to afford ethyl 2-[2-[5-(2,6-dibenzyloxy-3-pyridyl)-2-pyridyl]-2-azaspiro[3.3]heptan-6-yl]acetate (2.9 g, 3.94 mmol, 66.76% yield) as a gummy liquid. LCMS (ES⁺): m/z 550.79 [M+H]⁺.

### Step-6:

To a stirred solution of ethyl 2-[2-[5-(2,6-dibenzyloxy-3-pyridyl)-2-pyridyl]-2-azaspiro [3.3]heptan-6-yl] acetate (2.9 g, 5.28 mmol) in a mixture of THF (10 mL), methanol (6 mL) and water (4 mL) was added lithium hydroxide monohydrate (664.14 mg, 15.83 mmol, 0.44 mL) at 0° C. The reaction mixture was stirred at room temperature for 3 h. After complete consumption of starting material, the reaction mixture was concentrated under reduced pressure to obtain crude. The crude was diluted with water and acidified with 2N HCl to get precipitate. The precipitate was filtered, washed with water and dried to afford 2-[2-[5-(2,6-dibenzyloxy-3-pyridyl)-2-pyridyl]-2-azaspiro [3.3] heptan-6-yl] acetic acid (2.1 g, 2.99 mmol, 56.61% yield) as off-white solid. LCMS (ES⁺): m/z 522.44 [M+H]⁺.

### Step-7:

In a round bottom flask, to the stirred solution of 2-[2-[5-(2,6-dibenzyloxy-3-pyridyl)-2-pyridyl]-2-azaspiro [3.3] heptan-6-yl] acetic acid 10 (0.5 g, 0.958 mmol) in THE (2.5 mL), ethyl acetate (2.5 mL) and ethanol (10 mL) was added Palladium, 10% on carbon (0.5 g, 4.69 mmol) and the reaction was stirred at room temperature under H₂ atmosphere for 16 h. After complete consumption of starting material, the reaction mass was filtered through celite bed and washed with THE (50 mL). The filtrate was concentrated under reduced pressure and triturated with diethyl ether (10 mL) to give the crude product, which was purified by Prep-HPLC to afford 2-[2-[5-(2,6-dioxo-3-piperidyl)-2-pyridyl]-2-azaspiro [3.3] heptan-6-yl]acetic acid (0.023 g, 0.067 mmol, 21.50% yield) as pale pink solid. ¹H NMR (400 MHz, DMSO-d₆): δ 10.77 (s, 1H), 7.88 (d, J=2 Hz, 1H), 7.34 (dd, J=2.4, 8.4 Hz, 1H), 6.31 (d, J=8.2 Hz, 1H), 3.92 (s, 2H), 3.80 (s, 2H), 3.73-3.69 (m, 1H), 2.71-2.62 (m, 1H), 2.49-2.41 (m, 2H), 2.34-2.29 (m, 4H), 2.20-2.11 (m, 1H), 1.97-1.86 (m, 3H). LCMS (ES⁺): m/z 344.33 [M+H]⁺.

### Synthesis of 1-[6-(2,7-diazaspiro[3.5]nonan-7-yl)-5-fluoro-3-pyridyl]hexahydropyrimidine-2,4-dione

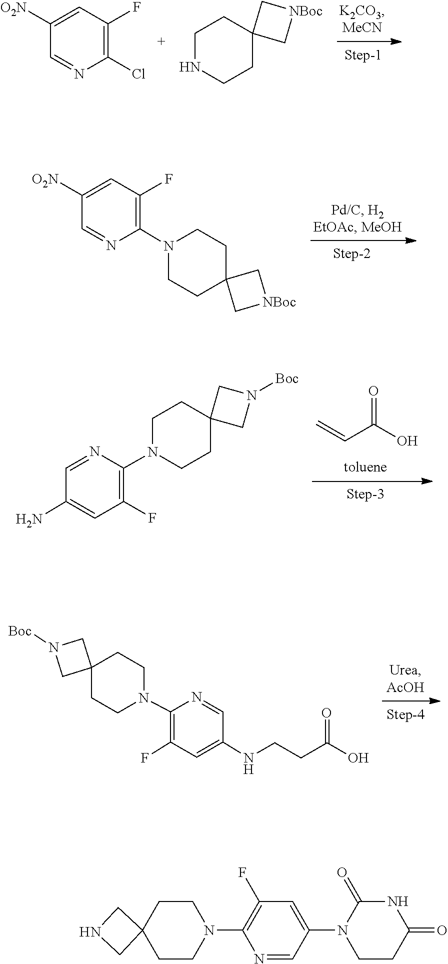

### Step-1:

A solution of 2-chloro-3-fluoro-5-nitro-pyridine (0.100 g, 566.47 μmol), tert-butyl 2,7-diazaspiro[3.5]nonane-2-carboxylate (163.74 mg, 623.11 μmol, HCl salt), potassium carbonate (313.16 mg, 2.27 mmol) in ACN (3 mL) was stirred at 80° C. for 16 h. The reaction mixture was extracted with cold water and ethyl acetate and the combined organic layers were washed with water, brine, dried over anhydrous Na₂SO₄, filtered and concentrated under reduced pressure. The crude compound was purified by column chromatography to afford tert-butyl 7-(3-fluoro-5-nitro-2-pyridyl)-2,7-diazaspiro[3.5]nonane-2-carboxylate (0.125 g, 313.88 μmol, 55.41% yield) as a light yellow solid. LCMS (ES⁺): m/z 367.32 [M+H]⁺.

### Step-2:

To a stirred solution of tert-butyl 7-(3-fluoro-5-nitro-2-pyridyl)-2,7-diazaspiro[3.5]nonane-2-carboxylate (2 g, 5.46 mmol) in ethyl acetate (5 mL) and ethanol (5 mL) was added 10% palladium on carbon (1.00 g) at room temperature. The reaction mixture was stirred under hydrogen atmosphere (balloon) for 16 h. Subsequently, it was filtered through celite bed and washed with ethyl acetate (15 mL). The filtrate was concentrated under reduced pressure to afford tert-butyl 7-(5-amino-3-fluoro-2-pyridyl)-2,7-diazaspiro[3.5]nonane-2-carboxylate (1.75 g, 4.63 mmol, 84.82% yield) as a brown semi solid. LCMS (ES⁺): m/z 337.64 [M+H]⁺.

### Step-3:

A solution of tert-butyl 7-(5-amino-3-fluoro-2-pyridyl)-2,7-diazaspiro[3.5]nonane-2-carboxylate (13 g, 38.64 mmol) and acrylic acid (2.78 g, 38.64 mmol, 2.65 mL) in toluene (100 mL) was stirred at 110° C. for 16 h. Upon completion of the reaction, the reaction mixture was cooled to room temperature and aqueous NaHCO₃ was added until the solution was basic. The aqueous layer was washed with ethyl acetate, and the aqueous layer was acidified with aqueous 2N HCl and extracted with DCM: Methanol (10%). The organic layer was then dried over sodium sulfate, filtered and concentrated to afford 3-[[6-(2-tert-butoxycarbonyl-2,7-diazaspiro[3.5]nonan-7-yl)-5-fluoro-3-pyridyl]amino]propanoic acid (1.2 g, 2.47 mmol, 6.39% yield) as a brown liquid. LCMS (ES⁺): m/z 409 [M+H]⁺.

### Step-4:

A solution of 3-[[6-(2-tert-butoxycarbonyl-2,7-diazaspiro[3.5]nonan-7-yl)-5-fluoro-3-pyridyl]amino]propanoic acid (1.5 g, 3.67 mmol), urea (1.10 g, 18.36 mmol, 826.06 μL) in acetic acid (25 mL) was stirred at 90 to 100° C. for 16 h. Upon completion of the reaction, the reaction mixture was quenched with NaHCO₃ and extracted with cold water and DCM: Methanol (10%). The combined organic layers were washed with water, brine, dried over anhydrous Na₂SO₄, filtered and concentrated under reduced pressure. To the residue was added 2 N HCl and THF (15 mL) and the mixture was stirred at 60° C. for 12 h. It was then concentrated and washed with THE and diethyl ether to give 1-[6-(2,7-diazaspiro[3.5]nonan-7-yl)-5-fluoro-3-pyridyl]hexahydropyrimidine-2,4-dione (0.750 g, 1.52 mmol, 41.42% yield, HCl salt) as a brown solid. LCMS (ES⁺): m/z 334.38 [M+H]⁺.

### Synthesis of 3-(6-(6-hydroxy-1,4-diazepan-1-yl)pyridin-3-yl)piperidine-2,6-dione

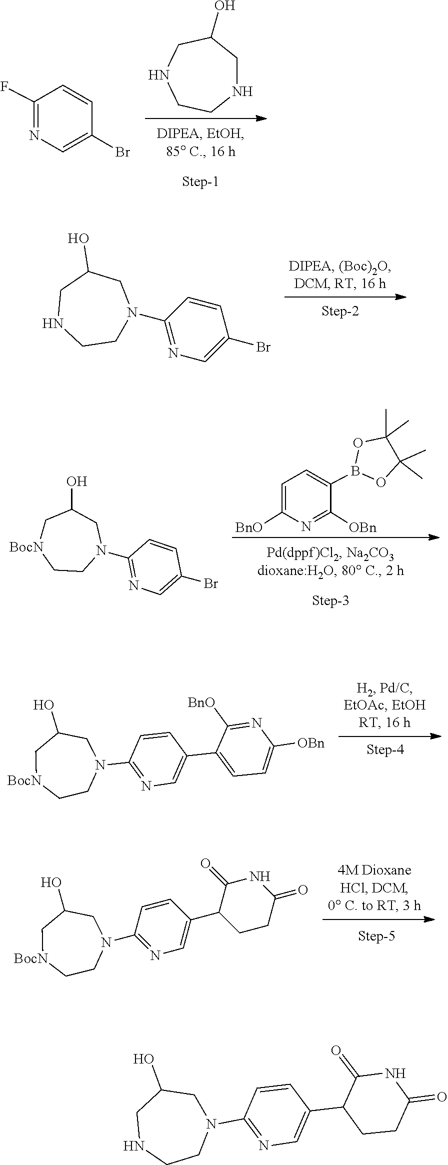

### Step-1:

To a stirring solution of 5-bromo-2-fluoro-pyridine (9 g, 51.14 mmol, 5.26 mL) in ethanol (150 mL) was added 1,4-diazepan-6-ol (3.96 g, 34.09 mmol) at room temperature. N-ethyl-N-isopropyl-propan-2-amine (19.83 g, 153.41 mmol, 26.72 mL) was then added dropwise. The reaction mixture was stirred at 85° C. for 16 h under nitrogen atmosphere. Upon completion of the reaction, the reaction mixture was cooled to RT and evaporated under reduced pressure. The residue was diluted with cold water (50 mL) and the aqueous layer was extracted with ethyl acetate (2×50 mL). The combined organic layer was separated, dried over sodium sulfate and concentrated under reduced pressure. The resulting crude (12 g) was triturated with pet ether (50 mL) and pentane (50 mL) to afford 1-(5-bromo-2-pyridyl)-1,4-diazepan-6-ol (5 g, 9.19 mmol, 26.94% yield) as a brown semi-solid. LCMS (ES⁺): m/z 274.34 [M+H]⁺.

### Step-2:

A stirring solution of 1-(5-bromo-2-pyridyl)-1,4-diazepan-6-ol (9 g, 35.07 mmol) in DCM (180 mL) was cooled to 0° C. under nitrogen atmosphere. N-ethyl-N-isopropyl-propan-2-amine (5.35 g, 41.36 mmol, 7.20 mL) and tert-butoxycarbonyl tert-butyl carbonate (7.22 g, 33.07 mmol, 7.60 mL) were added at 0° C. and the reaction mixture was stirred at RT for 16 h. Upon completion of the reaction, the reaction mixture was diluted with cold water (500 mL) and the aqueous layer was extracted with DCM (2×250 mL). The combined organic layer was separated, dried over sodium sulfate and concentrated under reduced pressure. The crude compound (17 g) was purified by column chromatography (230-400 mesh silica gel, 50% ethyl acetate in pet ether as mobile phase) to afford tert-butyl 4-(5-bromo-2-pyridyl)-6-hydroxy-1,4-diazepane-1-carboxylate (8.7 g, 16.36 mmol, 49.47% yield) as a brown solid. LCMS (ES⁺): m/z 374.40 [M+H]⁺.

### Step-3:

To a stirred solution of tert-butyl 4-(5-bromo-2-pyridyl)-6-hydroxy-1,4-diazepane-1-carboxylate (5 g, 13.43 mmol) and 2,6-dibenzyloxy-4-(4,4,5,5-tetramethyl-1,3,2-dioxaborolan-2-yl)pyridine (8.41 g, 20.15 mmol) in dioxane (80 mL) was added sodium carbonate (4.27 g, 40.29 mmol) in water (20 mL) and the mixture was purged with argon for 15 min. Then cyclopentyl(diphenyl)phosphane;dichloropalladium;iron (982.80 mg, 1.34 mmol) was added and purged with argon for another 5 min before the reaction mixture was stirred at 80° C. for 2 h. Upon completion of the reaction, the reaction mixture was cooled to RT, and concentrated under reduced pressure. The obtained crude product was diluted with cold water (80 mL) and extracted with ethyl acetate (3×80 mL). The combined organic layers were separated, dried over sodium sulfate and concentrated under reduced pressure. The crude compound (5 g) was purified by reverse phase column (25 to 35% ACN in 0.1% FA in water) and triturated with pentane to afford tert-butyl 4-[5-(2,6-dibenzyloxy-3-pyridyl)-2-pyridyl]-6-hydroxy-1,4-diazepane-1-carboxylate (2 g, 2.77 mmol, 20.65% yield) as a brown solid. LCMS (ES⁺): m/z 583.48 [M+H]⁺.

### Step-4:

To a stirred solution of tert-butyl 4-[5-(2,6-dibenzyloxy-3-pyridyl)-2-pyridyl]-6-hydroxy-1,4-diazepane-1-carboxylate (1.6 g, 2.75 mmol) in ethyl acetate (10 mL) and ethanol (10 mL) was added 10% palladium on carbon (1 g, 9.40 mmol) and the reaction mixture was stirred at 28° C. for 16 h under hydrogen atmosphere. Upon completion of the reaction, the reaction mixture was filtered through a pad of celite and washed with ethyl acetate (150 mL). The combined organic layer was concentrated under reduced pressure. The crude compound (1.5 g) was triturated with diethyl ether (50 mL) and pentane (50 mL) to afford tert-butyl 4-[5-(2,6-dioxo-3-piperidyl)-2-pyridyl]-6-hydroxy-1,4-diazepane-1-carboxylate (265 mg, 625.71 μmol, 22.79% yield) as brown solid. LCMS (ES⁻): m/z 402.90 [M−H]⁻

### Step-5:

To a stirred solution of tert-butyl 4-[5-(2,6-dioxo-3-piperidyl)-2-pyridyl]-6-hydroxy-1,4-diazepane-1-carboxylate (250 mg, 618.11 μmol) in DCM (5 mL) was added 4M HCl in dioxane (4M, 1 mL) at 0° C. The reaction mixture was stirred at 25° C. for 3 h. Upon completion of the reaction, the reaction mixture was concentrated under reduced pressure. The crude compound (0.25 g) was triturated with diethyl ether (10 mL) and pentane (10 mL) and the precipitate was filtered and dried to afford 3-(6-(6-hydroxy-1,4-diazepan-1-yl)pyridin-3-yl)piperidine-2,6-dione (205 mg, 571.98 μmol, 92.54% yield, HCl salt) as a purple solid. LCMS (ES⁻): m/z 302.78 [M−H]⁻

### 3-[5-fluoro-6-(6-hydroxy-1,4-diazepan-1-yl)-3-pyridyl]piperidine-2,6-dione

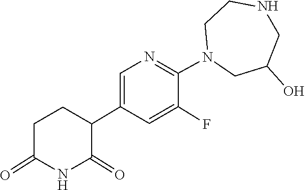 [SMILES: O=C1CCC(c2cnc(N3CCNCC(O)C3)c(F)c2)C(=O)N1]

This compound was prepared substantially following the synthesis of 3-(6-(6-hydroxy-1,4-diazepan-1-yl)pyridin-3-yl)piperidine-2,6-dione, using 5-bromo-2,3-difluoro-pyridine instead of 5-bromo-2-fluoro-pyridine in Step-1. LCMS (ES⁺): m/z 323.40 [M+H]⁺.

### Synthesis of 5-[[5-(2,6-dioxo-3-piperidyl)-2-pyridyl]amino]pentanoic acid

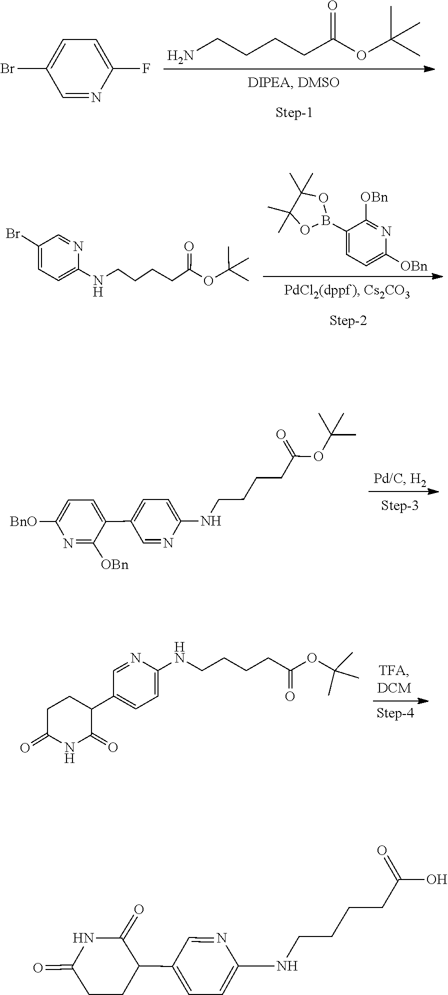

### Step-1:

To a stirred solution of 5-bromo-2-fluoro-pyridine (3.8 g, 21.59 mmol, 2.22 mL), DIPEA (13.95 g, 107.96 mmol, 18.8 mL) in DMSO (30 mL) was added tert-butyl 5-aminopentanoate (4.49 g, 25.91 mmol) slowly and heated at 100° C. for 16 h. After complete consumption of the starting material, the reaction mixture was quenched with ice cold water and extracted with EtOAc (2×100 mL), dried over anhydrous Na₂SO₄ and concentrated under reduced pressure. The crude product was purified by column chromatography (230-400 mesh SiO₂, 10-20% EtOAc in Pet ether) to afford tert-butyl 5-[(5-bromo-2-pyridyl)amino]pentanoate (1.5 g, 4.14 mmol, 19.16% yield) as an off-white solid. LCMS (ES⁺): m/z 330.16 [M+H]⁺.

### Step-2:

In a sealed tube, to a stirred solution of tert-butyl 5-[(5-bromo-2-pyridyl)amino]pentanoate (4.8 g, 14.58 mmol) in dioxane (40 mL) and water (10 mL) was added 2,6-dibenzyloxy-3-(4,4,5,5-tetramethyl-1,3,2-dioxaborolan-2-yl)pyridine (8.52 g, 20.41 mmol) and cesium carbonate (14.25 g, 43.74 mmol). The reaction mixture was degassed with argon for 10 minutes before [1,1′-Bis(diphenylphosphino)ferrocene]dichloropalladium(II) complex with dichloromethane (714.37 mg, 874.76 μmol) was added and stirred at 100° C. for 20 h. After complete consumption of starting material, the reaction was then cooled to room temperature and filtered through a short bed of celite. The filtrate was diluted with ethyl acetate (100 mL), washed with water, dried over anhydrous Na₂SO₄ and concentrated under reduced pressure. The crude product was purified by column chromatography using silica gel (230-400 mesh) and 10-20% ethyl acetate in hexane as eluent to afford tert-butyl 5-[[5-(2,6-dibenzyloxy-3-pyridyl)-2-pyridyl]amino]pentanoate (5.4 g, 7.54 mmol, 51.71% yield) as an off-white solid. LCMS (ES⁺): m/z 540.46 [M+H]⁺.

### Step-3:

To a stirred solution of tert-butyl 5-[[5-(2,6-dibenzyloxy-3-pyridyl)-2-pyridyl]amino]pentanoate (0.2 g, 0.37 mmol) in ethyl acetate (2.25 mL), THE (2.25 mL) and ethanol (0.5 mL) was added Palladium, 10% on carbon (0.2 g, 1.88 mmol) portion wise and the resulting mixture was stirred vigorously under hydrogen atmosphere (balloon) at room temperatures for 16 h. After complete consumption of starting material, the reaction mixture was filtered through celite bed and washed with ethyl acetate. The filtrate was concentrated and dried under high vacuum. The crude product was purified by column chromatography using 230-400 mesh silica gel and 100% ethyl acetate in hexane to afford tert-butyl 5-[[5-(2,6-dioxo-3-piperidyl)-2-pyridyl]amino]pentanoate (0.03 g, 81.90 μmol, 22.10% yield) as off-white solid. LCMS (ES⁺): m/z 362.39 [M+H]⁺.

### Step-4:

To a solution of tert-butyl 5-[[5-(2,6-dioxo-3-piperidyl)-2-pyridyl]amino]pentanoate (0.1 g, 276.68 μmol) in DCM (10 mL) was added TFA (1 mL, 12.98 mmol) at 0° C. over the period of 5 minutes and then stirred at room temperature for 4 h. After complete consumption of the starting material, the reaction mixture was concentrated under reduced pressure and co-distilled with toluene (10 mL) and diethyl ether (20×5 mL). The crude product was purified by Prep-HPLC to afford 5-[[5-(2,6-dioxo-3-piperidyl)-2-pyridyl]amino]pentanoic acid (0.02 g, 55.70 μmol, 20.13% yield) as a pink solid.

### Prep HPLC Conditions:

Column/dimensions: X-BRIDGEC8 (19*250 MM)

Mobile phase A: 5 mM Ammonium bicarbonate in water

Mobile phase B: 100% Acetonitrile

Gradient (Time/% B): 0/5, 2/5, 14.6/26, 14.70/98, 17.5/98, 17.9/5, 20/5

Flow rate: 17 mL/min

Solubility: Acetonitrile+THF+water.

LCMS (ES⁺): m/z 306.31 [M+H]⁺.

### 4-[[5-(2,6-dioxo-3-piperidyl)-2-pyridyl]amino]butanoic acid

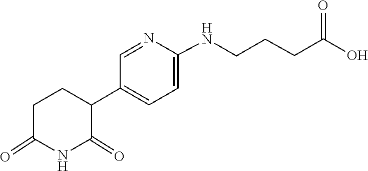 [SMILES: O=C(O)CCCNc1ccc(C2CCC(=O)NC2=O)cn1]

This compound was prepared substantially following the synthesis of 5-[[5-(2,6-dioxo-3-piperidyl)-2-pyridyl]amino]pentanoic acid, using tert-butyl 4-aminobutanoate instead of tert-butyl 5-aminopentanoate in Step-1. LCMS (ES⁺): m/z 292 [M+H]⁺.

### (3-[2-oxo-6-(4-piperidyl)-1,3-benzoxazol-3-yl]piperidine-2,6-dione

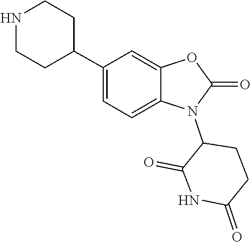 [SMILES: O=C1CCC(n2c(=O)oc3cc(C4CCNCC4)ccc32)C(=O)N1]

This compound was prepared according to the method described on page 190-191 of WO2021127586A1.

### Synthesis of 2-[1-[7-(2,6-dioxo-3-piperidyl)-9-methyl-8-oxo-purin-2-yl]-4-hydroxy-4-piperidyl]acetic acid

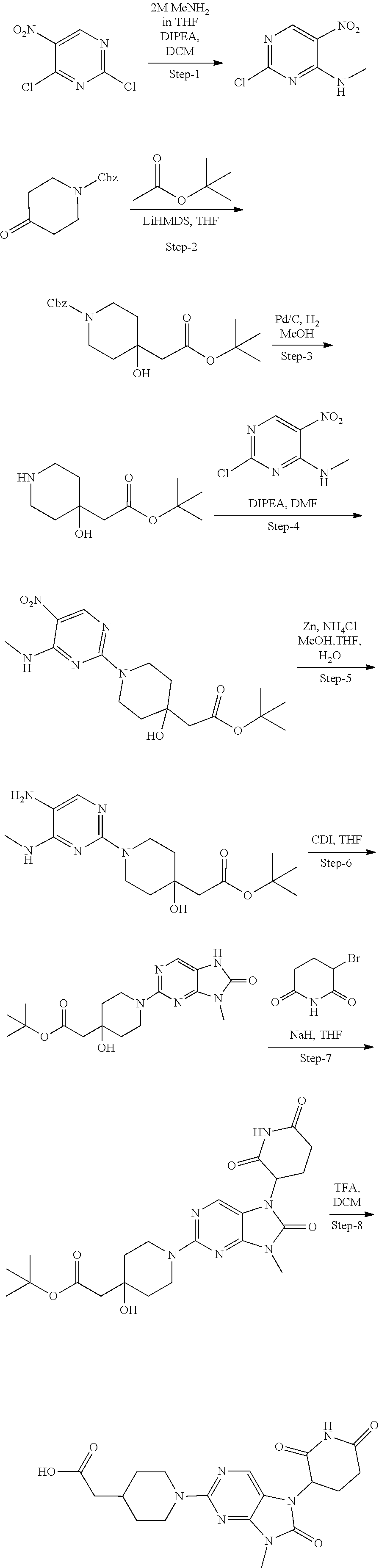

### Step-1:

A solution of 2,4-dichloro-5-nitro-pyrimidine (50 g, 257.76 mmol) in DCM (500 mL) was cooled to 0° C. under argon atmosphere. 2M Methyl amine in THF (2 M, 141 mL) and DIPEA (39.98 g, 309.32 mmol, 53.88 mL) were added and the reaction was stirred for 6 h at rt. Upon completion of the reaction, the reaction mixture was concentrated under reduced pressure, quenched with water (500 mL) and extracted with EtOAc (500 mL×2). The combined organic layer was washed with brine solution (200 mL), dried over anhydrous sodium sulfate, filtered and concentrated under reduced pressure. The residue was triturated with pet ether, filtered and dried to afford 2-chloro-N-methyl-5-nitro-pyrimidin-4-amine (40 g, 195.15 mmol, 75.71% yield) as an orange solid. LCMS (ES⁻): m/z 186.95 [M−H]⁻.

### Step-2:

A solution of lithium bis(trimethylsilyl)amide (1 M, 171.48 mL) was cooled to −78° C. before tert-butyl acetate (11.95 g, 102.89 mmol, 13.85 mL) in THE (200 mL) was added. After stirring at −78° C. for 1 h, a solution of benzyl 4-oxopiperidine-1-carboxylate (20 g, 85.74 mmol, 17.06 mL) in THE (200 mL) was added and the reaction mixture was stirred at −78° C. for 1 h and warmed to rt for 1 h. The reaction mixture was quenched with saturated aqueous solution of NH₄Cl (100 mL) and extracted with EtOAc (3×100 mL). The combined organic phase was washed with brine, dried over sodium sulfate, filtered and concentrated in vacuo to. The residue was purified by silica gel column chromatography (0-10% ethyl acetate/Pet ether) to give benzyl 4-(2-tert-butoxy-2-oxo-ethyl)-4-hydroxy-piperidine-1-carboxylate (17 g, 37.97 mmol, 44.28% yield). LCMS (ES⁺): m/z 292.31 [M−56]⁺

### Step-3:

A stirred solution of benzyl 4-(2-tert-butoxy-2-oxo-ethyl)-4-hydroxy-piperidine-1-carboxylate (16 g, 45.79 mmol) in methanol (160 mL) was degassed with nitrogen gas for 10 min. 10% Palladium on carbon (16 g, 45.79 mmol) was added and the reaction was stirred at RT under hydrogen atmosphere (balloon) for 16 h. Upon completion of the reaction, the reaction mixture was filtered through a pad of celite, and washed with methanol (100 mL). The filtrate was concentrated under reduced pressure to give tert-butyl 2-(4-hydroxy-4-piperidyl)acetate (6.5 g, 29.81 mmol, 65.10% yield) as white solid. LCMS (ES⁺): m/z 216.21 [M+H]⁺

### Step-4:

To a stirred solution of 2-chloro-N-methyl-5-nitro-pyrimidin-4-amine (10 g, 53.03 mmol) and tert-butyl 2-(4-hydroxy-4-piperidyl)acetate (11.42 g, 45.35 mmol, HCl salt) in DMF (60 mL) was added DIPEA (20.56 g, 159.09 mmol, 27.71 mL) and the reaction was stirred at 80° C. for 3 h. Upon completion of the reaction, the reaction mixture was quenched with ice cold water (200 mL). The solid was filtered and washed with excess water and dried to afford tert-butyl 2-[4-hydroxy-1-[4-(methylamino)-5-nitro-pyrimidin-2-yl]-4-piperidyl]acetate (20 g, 50.63 mmol, 95.47% yield) as an off white solid. LCMS (ES⁻): m/z 366.37 [M−H]⁻.

### Step-5:

To a stirred solution of tert-butyl 2-[4-hydroxy-1-[4-(methylamino)-5-nitro-pyrimidin-2-yl]-4-piperidyl]acetate (20 g, 54.44 mmol) in methanol (200 mL) and THE (600 mL) was added ammonium Chloride (58.24 g, 1.09 mol) in water (200 mL) at 0° C. Then zinc (35.60 g, 544.37 mmol) was added portionwise and reaction mixture was stirred at 25° C. for 2 h. After completion the reaction, the reaction mixture was filtered through a pad of celite and washed with DCM (200 mL). The filtrate was concentrated under reduced pressure, quenched with water (200 mL) and then extracted with DCM (200 mL×2). The combined organic layer was washed with brine solution (100 mL), dried over anhydrous sodium sulfate, filtered and concentrated under reduced pressure to afford tert-butyl 2-[1-[5-amino-4-(methylamino)pyrimidin-2-yl]-4-hydroxy-4-piperidyl]acetate (15 g, 31.80 mmol, 58.42% yield) as a black gum. LCMS (ES⁺): m/z 338.53 [M+H]⁺.

### Step-6:

A solution of tert-butyl 2-[1-[5-amino-4-(methylamino)pyrimidin-2-yl]-4-hydroxy-4-piperidyl]acetate (15 g, 44.46 mmol) in THF (200 mL) was cooled to 0° C. under argon atmosphere and 1,1′-carbonyldiimidazole (18.02 g, 111.14 mmol) was added portionwise. Then the reaction mixture was stirred at 25° C. for 3 h. After the completion of reaction, the reaction mixture was diluted with water (300 mL) and extracted with ethyl acetate (2×300 mL). The organic layer was separated, washed with brine solution (200 mL) dried over Na₂SO₄ and concentrated in vacuo. The crude was purified by column chromatography (100-200 mesh silica gel, 0-80% EtOAc in pet ether as eluent) to afford tert-butyl 2-[4-hydroxy-1-(9-methyl-8-oxo-7H-purin-2-yl)-4-piperidyl]acetate (7.6 g, 20.29 mmol, 45.63% yield) as an off white solid. LCMS (ES⁺): m/z 364.58 [M+H]⁺.

### Step-7:

To a stirred solution of tert-butyl 2-[4-hydroxy-1-(9-methyl-8-oxo-7H-purin-2-yl)-4-piperidyl]acetate (6.0 g, 16.51 mmol) in THE (200 mL) 0° C. under argon atm, NaH (3.17 g, 132.08 mmol) was added and stirred for 1 h. Then 3-bromopiperidine-2,6-dione (15.85 g, 82.55 mmol) was added and the reaction was stirred at 60° C. for 16 h. Upon completion of the reaction, the reaction mixture was quenched with ammonium chloride solution (200 mL) and extracted with EtOAc (200 mL×2). The combined organic layer was washed with brine solution (200 mL), dried over anhydrous sodium sulfate, filtered through a pad of celite and concentrated under reduced pressure. The residue was triturated with ethyl acetate, filtered, and dried to afford tert-butyl 2-[1-[7-(2,6-dioxo-3-piperidyl)-9-methyl-8-oxo-purin-2-yl]-4-hydroxy-4-piperidyl]acetate (3.0 g, 6.01 mmol, 36.38% yield) as an ash colored solid. LCMS (ES⁺): m/z 475.66 [M+H]⁺.

### Step-8:

To a stirred solution of tert-butyl 2-[1-[7-(2,6-dioxo-3-piperidyl)-9-methyl-8-oxo-purin-2-yl]-4-hydroxy-4-piperidyl]acetate (0.20 g, 421.49 μmol) in DCM (4.00 mL) was added trifluoroacetic acid (4 M, 2 mL) at 0° C. and the reaction mixture was stirred at room temperature for 1 h. After completion of the reaction, the reaction mixture was concentrated under reduced pressure. The resulting crude was triturated with diethyl ether (50 mL) and dried under reduced pressure to afford 2-[1-[7-(2,6-dioxo-3-piperidyl)-9-methyl-8-oxo-purin-2-yl]-4-hydroxy-4-piperidyl]acetic acid (0.18 g, 317.79 μmol, 75.40% yield, TFA salt) as a yellow solid. LCMS (ES⁺): m/z 419.20 [M+H]⁺.

### Synthesis of 2-(1-(3-(2,6-dioxopiperidin-3-yl)-1-methyl-2-oxo-2,3-dihydro-1H-imidazo[4,5-b]pyridin-6-yl)-4-hydroxypiperidin-4-yl)acetic acid

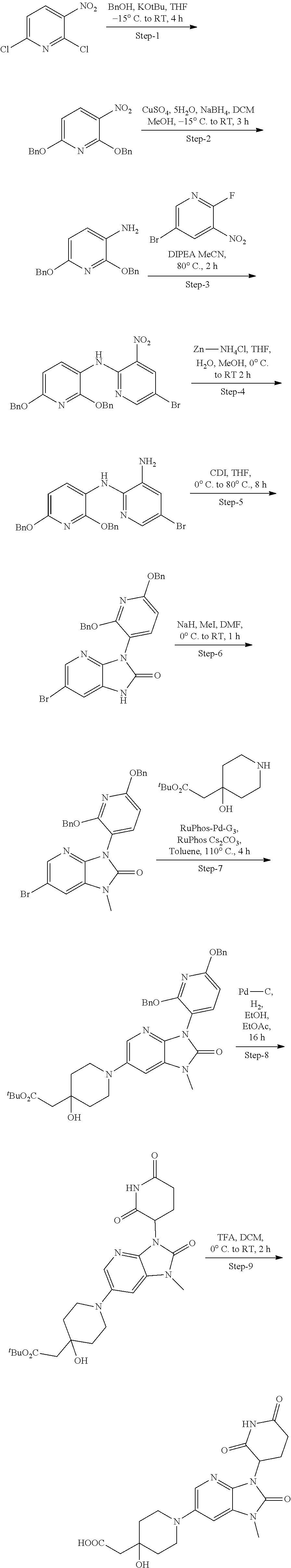

### Step-1:

To a stirred solution of benzyl alcohol (75.65 g, 699.53 mmol) in THF (250 mL) was cooled to −15° C. was added potassium tert-butoxide (70 g, 621.80 mmol) in portions and the reaction mixture was stirred at room temperature for 0.5 h. This reaction mixture was added to a solution of 6-dichloro-3-nitropyridine (50.0 g, 259.08 mmol) in THF (250 mL) dropwise at −15° C. The reaction mixture was stirred at −15° C. for 2 h then poured into cold water (1 L) and stirred for 30 min. The precipitated solid was filtered and washed with water (1 L) then triturated with diethyl ether (400 mL) and pentane (200 mL) to afford 2,6-bis(benzyloxy)-3-nitropyridine (2) (150 g, 423.68 mmol, 81.76% yield) as off-white solid. LCMS (ES⁻): m/z 335.18 [M−H]⁻.

### Step-2:

To a stirred solution of 2,6-bis(benzyloxy)-3-nitropyridine (120 g, 356.78 mmol) in DCM (1.0 L) was added a solution of copper (II) sulphate pentahydrate (17.82 g, 71.26 mmol) in methanol (1 L) dropwise. The reaction mixture was cooled to −10° C. and sodium borohydride (53.99 g, 1.43 mmol) portion wise at −10° C. The reaction mixture was stirred at RT for 3 h then diluted with cold water (1 L) and filtered through celite bed. The filtrate was extracted with DCM (2×1 L). The separated organic layer was washed with water (2×500 mL) and brine (500 mL). The combined organic layer was separated, dried over sodium sulphate and concentrated under reduced pressure and trituration with pet ether (500 mL) afforded 2,6-bis(benzyloxy)pyridin-3-amine (70.0 g, 62.12% yield) as black gummy liquid. LCMS (ES⁺): m/z 307.25 [M+H]⁺.

### Step-3:

To a stirred solution of 2,6-bis(benzyloxy)pyridin-3-amine (0.25 g, 816.04 μmol) in ACN (8 mL) was added DIPEA (0.26 g, 2.04 mmol) at room temperature and stirred for 10 min then 5-bromo-2-flouro-3-nitropyridine (0.19 g, 897.44 μmol) was added portion wise. After addition the reaction mixture was stirred at 80° C. for 2 h. Upon completion, the reaction mixture was cooled to room temperature and poured in cold water (30 mL) and stirred for 10 min. The precipitated solid was filtered and washed with water (10 mL) and trituration with diethyl ether (10 mL) gave 1-(2,6-bis(benzyloxy)pyridin-3-yl)-5-bromo-1,3-dihydro-2H-benzo[d]imidazol-2-one (0.32 g, 531.72 μmol, 65.16% yield) as pale red solid. LCMS (ES⁺): m/z 507.24 [M+H]⁺.

### Step-4:

To a stirred solution of 1-(2,6-bis(benzyloxy)pyridin-3-yl)-5-bromo-1,3-dihydro-2H-benzo[d]imidazol-2-one (0.1 g, 197.11 μmol) in methanol (1 mL), THE (3 mL) and water (1 mL) was cooled to 0° C. was added zinc dust (0.129 g, 1.97 mmol) portion wise, follow by ammonium chloride (0.21 g, 3.94 mmol) portion wise at 0° C. The reaction mixture was stirred at RT for 2 h. The reaction mixture was filtered through a celite bed, which was washed with ethyl acetate (10 mL). The filtrate was evaporated under reduced pressure then dissolved in cold water (10 mL) and extracted with ethyl acetate (3×10 mL) and washed with brine (3×10 mL). The combined organic layer was dried over sodium sulphate and concentrated. Trituration with pet ether (10 mL) gave N2-(2,6-bis(benzyloxy)pyridin-3-yl)-5-bromopyridine-2,3-diamine (0.07 g, 124.65 μmol, 63.24% yield) as pale red gummy liquid. LCMS (ES⁺): m/z 478.31 [M+H]⁺.

### Step-5:

To a stirred solution of N2-(2,6-bis(benzyloxy)pyridin-3-yl)-5-bromopyridine-2,3-diamine (20.0 g, 41.9 mmol) in THE (200 mL) was cooled to 0° C. was added CDI (27.17 g, 167.59 mol) portion wise and stirred the reaction mixture at 80° C. for 8 h. The reaction mixture was cooled to RT and diluted with cold water (300 mL) and extracted with ethyl acetate (3×200 mL). The separated organic layer was washed with water (200 mL) and brine (100 mL). The combined organic layer was, dried over sodium sulphate and concentrated under reduced pressure to afford crude product. Trituration with diethyl ether (3×100 mL) gave 3-(2,6-bis(benzyloxy)pyridin-3-yl)-6-bromo-1,3-dihydro-2H-imidazo[4,5-b]pyridin-2-one (19.0 g, 82.44% yield) as an off white solid. LCMS (ES⁺): m/z 505.21 [M+H]⁺.

### Step-6:

To a stirred solution of 3-(2,6-bis(benzyloxy)pyridin-3-yl)-6-bromo-1,3-dihydro-2H-imidazo[4,5-b]pyridin-2-one (0.5 g, 993.35 μmol) in DMF (5 mL) was added sodium hydride (60% dispersion in mineral oil) (46.7 mg, 1.99 mmol) portion wise and the reaction mixture was stirred at 0° C. for 0.5 h. Methyl iodide (92.76 μL, 1.49 mmol) was added dropwise at 0° C. and the reaction mixture was stirred at 28° C. for 1 h. The reaction mixture was quenched with cold aqueous ammonium chloride solution (30 mL) and stirred for 30 min. The precipitated solid was filtered off and washed with cold water (30 mL). The obtained precipitate was dried under reduced pressure then triturated with diethyl ether (10 mL) to give 3-(2,6-bis(benzyloxy)pyridin-3-yl)-6-bromo-1-methyl-1,3-dihydro-2H-imidazo[4,5-b]pyridin-2-one (0.3 g, 53.28% yield) as pale brown solid. LCMS (ES⁺): m/z 517.41 [M+H]⁺.

### Step-7:

To a stirred solution of 3-(2,6-bis(benzyloxy)pyridin-3-yl)-6-bromo-1-methyl-1,3-dihydro-2H-imidazo[4,5-b]pyridin-2-one (0.1 g, 193.28 μmol) and tert-butyl 2-(4-hydroxypiperidin-4-yl)acetate (85 mg, 394.82 mmol) in toluene (1 mL) and was added cesium carbonate (126 mg, 386.57 μmol) portion wise at 28° C. The reaction mixture was degassed under argon atmosphere for 10 min, before the addition of RuPhos (20 mg, 42.86 μmol) and RuPhos-Pd-G3 (10 mg, 11.96 μmol). After addition, the reaction was degassed again under argon atmosphere for 10 min and stirred at 110° C. for 4 h. The reaction mixture was cooled to room temperature and filtered through celite. The celite bed was washed with ethyl acetate (20 mL). The filtrate was washed with water (30 mL) and brine solution (10 mL). The organic layer was dried over anhydrous sodium sulphate and evaporated under reduced pressure. Purification by silica gel column chromatography (230-400 mesh, 0-40% Ethyl acetate in pet ether as an eluent) gave tert-butyl 2-(1-(3-(2,6-bis(benzyloxy)pyridin-3-yl)-1-methyl-2-oxo-2,3-dihydro-1H-imidazo[4,5-b]pyridin-6-yl)-4-hydroxypiperidin-4-yl)acetate (10 mg, 11.3 μmol, 5.85% yield) as brown gummy liquid. LCMS (ES⁺): m/z 652.57 [M+H]⁺.

### Step-8:

A stirred solution of tert-butyl 2-(1-(3-(2,6-bis(benzyloxy)pyridin-3-yl)-1-methyl-2-oxo-2,3-dihydro-1H-imidazo[4,5-b]pyridin-6-yl)-4-hydroxypiperidin-4-yl)acetate (0.19 g, 291.52 μmol) in ethanol (10 mL) and ethyl acetate (3 mL) was added to parr shaker vessel. 10% palladium on carbon (50% wet basis, 0.19 g) was added and the reaction mixture was stirred at 70 Psi hydrogen pressure for 16 h. The reaction mixture was filtered through celite and washed with ethanol (10 mL). The filtrate was concentrated under reduced pressure and trituration with diethyl ether (5 mL) and pentane (5 mL) gave tert-butyl 2-(1-(3-(2,6-dioxopiperidin-3-yl)-1-methyl-2-oxo-2,3-dihydro-1H-imidazo[4,5-b]pyridin-6-yl)-4-hydroxypiperidin-4-yl)acetate (0.12 g, 148.86 μmol, 51.06% yield) as brown solid. LCMS (ES⁺): m/z 474.49 [M+H]⁺.

### Step-9:

To a stirred solution of tert-butyl 2-(1-(3-(2,6-dioxopiperidin-3-yl)-1-methyl-2-oxo-2,3-dihydro-1H-imidazo[4,5-b]pyridin-6-yl)-4-hydroxypiperidin-4-yl)acetate (0.12 g, 253.42 μmol) in DCM (5 mL) was added triflouroacetic acid (1 mL) at 0° C. The reaction mixture was stirred at 25° C. for 2 h then concentrated under reduced pressure. Purification by prep-HPLC afforded 2-(1-(3-(2,6-dioxopiperidin-3-yl)-1-methyl-2-oxo-2,3-dihydro-1H-imidazo[4,5-b]pyridin-6-yl)-4-hydroxypiperidin-4-yl)acetic acid (29.5 mg, 55.18 μmol, 21.77% yield, TFA salt) as orange gummy liquid. LCMS (ES⁺): m/z 418.25 [M+H]⁺. ¹H NMR (400 MHz, DMSO-d₆): δ 12.02 (bs, 1H), 11.08 (s, 1H), 7.68 (bs, 1H), 7.40 (bs, 1H), 5.34-5,29 (m, 1H), 3.39 (bs, 5H), 3.38-3.27 (m, 2H), 2.97-2.73 (m, 2H), 2.68-2.655 (m, 2H), 2.48 (s, 2H), 2.17-2.08 (bs, 1H), 1.99-1.89 (bs, 2H), 1.77-1.29 (bs, 2H).

### Prep-HPLC Condition:

Column/dimensions: Sunfire C18 (19*300, 7 um)

Mobile phase A: 0.05% TFA IN water

Mobile Phase B: Acetonitrile

Gradient (Time/% B): 0/5,3/5,10/20.10.1/100,13/100,13.1/5,13.5/5

Flow rate: 17 mL/min.

Solubility: THF+WATER+CAN

### Synthesis of 1-(6-((3S,4S)-3-hydroxypiperidin-4-yl)-1-methyl-1H-indazol-3-yl) dihydropyrimidine-2,4(1H,3H)-dione

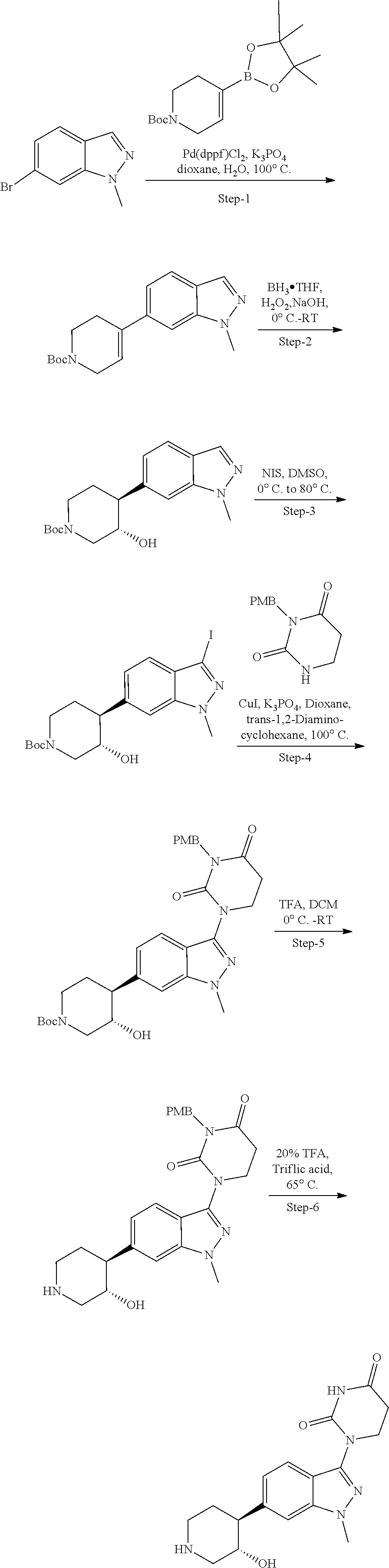

### Step-1:

To a stirred solution of 6-bromo-1-methyl-indazole (10 g, 47.38 mmol) in dioxane (100 mL) and water (40 mL) was added tert-butyl 4-(4,4,5,5-tetramethyl-1,3,2-dioxaborolan-2-yl)-3,6-dihydro-2H-pyridine-1-carboxylate (17.58 g, 56.86 mmol) portion wise. Potassium phosphate tribasic anhydrous (30.17 g, 142.14 mmol) was added at RT under nitrogen gas. The reaction mixture was degassed with argon for 10 minutes. After degassing, PdCl₂(dppf)CH₂Cl₂ (3.87 g, 4.74 mmol) was added and the reaction mixture was stirred at 80° C. for 16 h. The reaction mixture was concentrated under reduced pressure, diluted with water (100 mL) and extracted with ethyl acetate (2×100 mL). The combined organic layer dried over anhydrous sodium sulphate, concentrated under reduced pressure and purification by column chromatography (silica gel mesh 100-200,20-30% ethyl acetate in pet ether) gave tert-butyl 4-(1-methyl-1H-indazol-6-yl)-3,6-dihydropyridine-1(2H)-carboxylate (8 g, 17.83 mmol, 37.62% yield) as a brown liquid. LCMS (ES⁺): m/z 314.27 [M+H]⁺.

### Step-2:

To a stirred solution of tert-butyl 4-(1-methyl-1H-indazol-6-yl)-3,6-dihydropyridine-1(2H)-carboxylate (8 g, 25.53 mmol) in THE (20 mL) was added borane tetrahydrofuran complex solution 1.0 M in THE (1 M, 63.82 mL) dropwise at −10° C. and the reaction mixture was stirred at RT for 1 h. After that the reaction mixture was cooled to −10° C. and quenched with 35% hydrogen peroxide (3.47 g, 102.11 mmol, 3.16 mL) followed by sodium hydroxide (1 M, 51.05 mL) and again stirred the reaction mixture at RT for 16 h. The reaction mixture was quenched with aq. saturated sodium sulphite (100 mL) solution and extracted with ethyl acetate (2×100 mL). The combined organic layer was dried over anhydrous Na₂SO₄, concentrated under reduced pressure and purification by column chromatography (silica gel, 10-20% ethyl acetate in pet ether) gave tert-butyl (3S,4S)-3-hydroxy-4-(1-methyl-1H-indazol-6-yl)piperidine-1-carboxylate (6 g, 17.28 mmol, 67.68% yield) as a white solid. LCMS (ES⁺): m/z 332.37 [M+H]⁺.

### Step-3:

To a stirred solution of tert-butyl (3S,4S)-3-hydroxy-4-(1-methyl-1H-indazol-6-yl)piperidine-1-carboxylate (6 g, 18.1 mmol) in DMSO (60 mL) was added N-iodosuccinimide (6.11 g, 27.16 mmol) portion wise at 0° C. The reaction mixture was stirred at 80° C. for 3 h. The reaction mixture was quenched with cold water (100 mL) and extracted with ethyl acetate (2×100 mL). The combined organic layer was dried over anhydrous Na₂SO₄, concentrated under reduced pressure and purification by column chromatography (silica, 100-200, 20-30% ethyl acetate in pet ether) gave tert-butyl (3S,4R)-3-hydroxy-4-(3-iodo-1-methyl-1H-indazol-6-yl)piperidine-1-carboxylate (6.5 g, 12.93 mmol, 71.44% yield) as an yellow solid. LCMS (ES⁺): m/z 458.19 [M+H]⁺.

### Step-4:

To a stirred solution of tert-butyl (3S,4R)-3-hydroxy-4-(3-iodo-1-methyl-1H-indazol-6-yl)piperidine-1-carboxylate (6 g, 13.12 mmol) and 3-(4-methoxybenzyl)dihydropyrimidine-2,4(1H,3H)-dione (4.61 g, 19.68 mmol) in dioxane (60 mL) was added potassium phosphate tribasic (5.57 g, 26.24 mmol) and copper (I) iodide (1.25 g, 6.56 mmol) and trans-1,2-diaminocyclohexane (749.1 mg, 6.56 mmol) portion wise at RT under nitrogen gas. The reaction mixture was degassed with argon for 10 minutes and stirred at 100° C. for 6 h. The reaction mixture was cooled to RT and evaporated under reduced pressure. The material was diluted with water (100 mL) and extracted with ethyl acetate (2×100 mL). The combined organic layer was washed with brine (50 mL) and dried over anhydrous sodium sulphate, evaporated under reduced pressure and purification by column chromatography over silica gel (100-200 mesh, 30-50% ethyl acetate in pet-ether as eluent) gave tert-butyl (3S,4S)-3-hydroxy-4-(3-(3-(4-methoxybenzyl)-2,4-dioxotetrahydropyrimidin-1(2H)-yl)-1-methyl-1H-indazol-6-yl)piperidine-1-carboxylate (5.5 g, 8.07 mmol, 61.54% yield) as white solid. LCMS (ES⁺): m/z 564.52 [M+H]⁺. ¹H NMR (400 MHz, DMSO-d₆): δ 7.48 (s, 1H), 7.46 (s, 1H), 7.24 (d, J=8.4 Hz, 2H), 7.03 (d, J=8.8 Hz, 1H), 6.87 (d, J=8.4 Hz, 2H), 4.84 (s, 3H), 4.15 (bs, 1H), 3.95 (bs, 1), 3.93 (s, 3H), 3.90 (t, J=6.8 Hz, 1H), 3.89-3.86 (m, 1H). 3.72 (s, 3H), 3.62-3.57 (m, 1H), 2.94 (t, J=6.8 Hz, 2H), 2.86-2.56 (bs, 3H), 1.75-1.63 (m, 2H), 1.43 (s. 9H).

### Step-5:

A stirred solution of tert-butyl (3S, 4S)-3-hydroxy-4-[3-[3-[(4-methoxyphenyl)methyl]-2,4-dioxo-hexahydropyrimidin-1-yl]-1-methyl-indazol-6-yl]piperidine-1-carboxylate (1.5 g, 2.66 mmol) in DCM (20 mL) was cooled to 0° C. and trifluoroacetic acid, 99% (910.33 mg, 7.98 mmol, 615.09 μL) was added drop wise and the reaction was then stirred at 25° C. for 2 h. The reaction mixture was concentrated under reduced pressure and trituration with diethyl ether (25 mL) gave 3-[(4-methoxyphenyl)methyl]-1-[1-methyl-6-[(3S,4S)-3-hydroxy-4-piperidyl]indazol-3-yl]hexahydropyrimidine-2,4-dione (0.055 g, 94.89 μmol, 3.57% yield) as white solid. LCMS (ES⁺): m/z 464.34 [M+H]⁺. ¹H NMR (400 MHz, DMSO-d₆): δ 8.81-8.73 (bs, 1H), 8.64-8.52 (bs, 1H), 7.55 (d, J=8.4 Hz, 1H), 7.38 (s, 1H), 7.24 (d, J=8.4 Hz, 2H), 7.0 (d, J=8.4 Hz, 1H), 6.88 (d, J=8.4 Hz, 2H), 5.27 (bs, 1H), 4.85 (s, 2H), 4.01-3.86 (m, 6H), 3.73 (s, 3H), 3.39 (bs, 2H), 3.04-2.82 (m, 3H), 2.76-2.64 (m, 2H), 1.94 (bs, 2H).

### Step-6:

To a stirred solution of 3-[(4-methoxyphenyl)methyl]-1-[1-methyl-6-[(3S,4S)-3-hydroxy-4-piperidyl]indazol-3-yl]hexahydropyrimidine-2,4-dione (0.5 g, 0.865 mmol, TFA salt) in TFA (4.70 mL) was added trifluoromethanesulfonic acid (649.63 mg, 4.33 mmol, 380.34 μL) drop wise. The reaction mixture was stirred at 65° C. for 16 h. The reaction mixture was cooled to room temperature, evaporated under reduced pressure and triturated with diethyl ether (10 mL) and pentane (15 mL). The crude material was purified by prep-HPLC to afford 1-(6-((3S,4S)-3-hydroxypiperidin-4-yl)-1-methyl-1H-indazol-3-yl) dihydropyrimidine-2,4(1H,3H)-dione (22 mg, 48.05 μmol, 5.55% yield, TFA salt) as an off-white solid. LCMS (ES⁺): m/z 344.36 [M+H]⁺. ¹H NMR (400 MHz, DMSO-d₆): δ 10.56 (s, 1H), 8.76 (brs, 1H), 8.55 (brs, 1H), 7.61 (d, J=8.4 Hz, 1H), 7.38 (s, 1H), 7.00 (d, J=8.4 Hz, 1H), 5.27 (d, J=6.0 Hz, 1H), 3.98-3.90 (m, 6H), 3.40-3.39 (m, 2H), 3.0-2.97 (m, 1H), 2.84-2.71 (m, 4H), 1.94-1.86 (m, 2H).

### Prep-HPLC Purification:

Column/dimensions: X-BRIDGE C18 (19*250*5 um)

Mobile phase A: 0.1% TFA IN WATER

Mobile phase B: 100% ACN (org)

Gradient (Time/% B): 0/5,3/5,7/25,10/25,10.1/100,14/100,14.1/5,18/5

Flow/rate: 18 ml/min.

Solubility: Acetonitrile+THF+WATER

### Synthesis of 3-(6-((3R,4S)-3-hydroxypiperidin-4-yl)-1-methyl-1H-indazol-3-yl)piperidine-2,6-dione

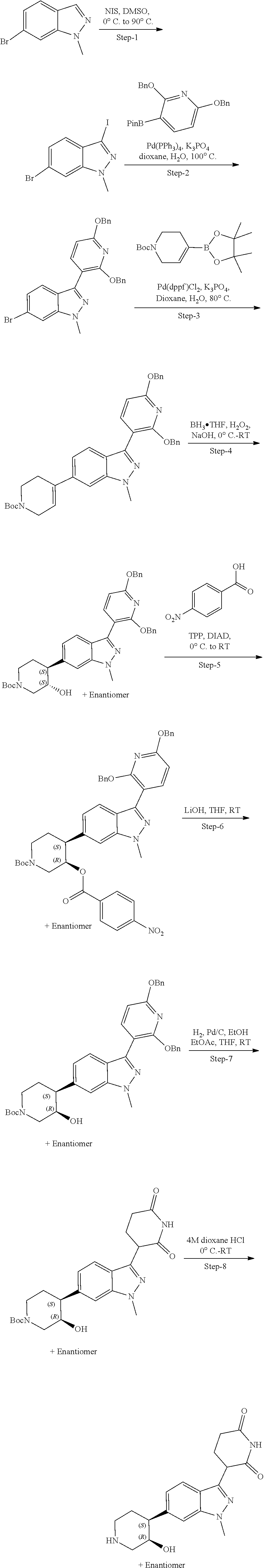

### Step-1:

To stirred solution of 6-bromo-1-methyl-indazole (20 g, 94.76 mmol) in DMSO (200 mL) was added N-iodosuccinimide (25.58 g, 113.71 mmol) portion wise at 0° C. The reaction mixture was stirred at 90° C. for 16 h. After completion, the reaction mixture was quenched with ice cold saturated sodium thiosulfate (200 mL) solution. The solid precipitate was filtered and dried to afford 6-bromo-3-iodo-1-methyl-indazole (30 g, 58.56 mmol, 61.79% yield) as yellow solid. LCMS (ES⁺): m/z 337.15 [M+H]⁺.

### Step-2:

To a stirred solution of 6-bromo-3-iodo-1-methyl-indazole (15 g, 44.52 mmol) in dioxane (320 mL) and water (80 mL) was added 2,6-dibenzyloxy-3-(4,4,5,5-tetramethyl-1,3,2-dioxaborolan-2-yl)pyridine (18.58 g, 44.52 mmol) and potassium phosphate tribasic (28.35 g, 133.55 mmol) at RT under nitrogen gas. The reaction mixture was degassed with argon for 10 minutes. Tetrakis (triphenylphosphine) palladium(0) (5.14 g, 4.45 mmol) was added and the reaction mixture was heating at 100° C. for 16 h while monitoring with TLC and LCMS. After completion, the reaction mixture was cooled to RT and evaporated under reduced pressure. The obtained crude was diluted with water (250 mL) and extracted with ethyl acetate (2×200 mL). The combined organic layer was washed with brine (250 mL) and dried over anhydrous sodium sulphate and evaporated under reduced pressure. The crude material was purified by column chromatography over silica gel (230-400 mesh, 0-30% ethyl acetate in pet-ether as eluent) to give 6-bromo-3-(2,6-dibenzyloxy-3-pyridyl)-1-methyl-indazole (7 g, 11.89 mmol, 26.71% yield) as yellow solid. LCMS (ES⁺): m/z 500.18 [M+H]⁺.

### Step-3:

To a stirred solution of 6-bromo-3-(2,6-dibenzyloxy-3-pyridyl)-1-methyl-indazole (4.5 g, 8.99 mmol) in dioxane (40 mL) and water (10 mL) was added tert-butyl 4-(4,4,5,5-tetramethyl-1,3,2-dioxaborolan-2-yl)-3,6-dihydro-2H-pyridine-1-carboxylate (4.17 g, 13.49 mmol) portion wise. Potassium phosphate tribasic anhydrous was added (1.91 g, 8.99 mmol) at RT under nitrogen gas. The reaction mixture was degassed with argon for 10 minutes, then PdCl₂(dppf)CH₂Cl₂ (734.41 mg, 0.899 mmol) was added and the reaction mixture was stirred at 80° C. for 16 h. The reaction mixture was concentrated under reduced pressure, diluted with water (150 mL) and extracted with ethyl acetate (3×100 mL). The combined organic layer was dried over anhydrous sodium sulphate, concentrated under reduced pressure and purification by column chromatography (100-200 mesh silica gel, 20-30% ethyl acetate in pet ether) gave tert-butyl 4-[3-(2,6-dibenzyloxy-3-pyridyl)-1-methyl-indazol-6-yl]-3,6-dihydro-2H-pyridine-1-carboxylate (4.5 g, 4.89 mmol, 54.35% yield) as a brown liquid. LCMS (ES⁺): m/z 603.43 [M+H]⁺.

### Step-4:

To a stirred solution of tert-butyl 4-[3-(2,6-dibenzyloxy-3-pyridyl)-1-methyl-indazol-6-yl]-3,6-dihydro-2H-pyridine-1-carboxylate (4.50 g, 7.47 mmol) in THF (40 mL) was added borane tetrahydrofuran complex solution 1.0 M in THE (1 M, 18.67 mL) dropwise at 0° C. and the reaction mixture was stirred at RT for 1 h. The reaction mixture was cooled to 0° C. and quenched with 35% hydrogen peroxide (1.02 g, 29.86 mmol, 923.36 μL) followed by sodium hydroxide (1 M, 14.93 mL) and again stirred the reaction mixture at RT for 16 h. The reaction mixture was quenched with a aq. saturated sodium sulphite (50 mL) solution and extracted with ethyl acetate (2×100 mL). The combined organic layer was dried over anhydrous Na₂SO₄, concentrated under reduced pressure and purified by column chromatography (100-200 mesh silica gel, 10-20% ethyl acetate in pet ether) to give tert-butyl (3S)-4-[3-(2,6-dibenzyloxy-3-pyridyl)-1-methyl-indazol-6-yl]-3-hydroxy-piperidine-1-carboxylate (3.2 g, 4.02 mmol, 53.86% yield) as a sticky colourless liquid. LCMS (ES⁺): m/z 621.49 [M+H]⁺.

### Step-5:

To a stirred solution of tert-butyl (3S)-4-[3-(2,6-dibenzyloxy-3-pyridyl)-1-methyl-indazol-6-yl]-3-hydroxy-piperidine-1-carboxylate (4.50 g, 7.47 mmol) and 4-nitrobenzoic acid (1.62 g, 9.67 mmol) in THE (50 mL) was added triphenylphosphine (3.38 g, 12.89 mmol) at 0° C. and the reaction mixture was stirred for 10 min, followed by the addition of diisopropyl azodicarboxylate (2.61 g, 12.89 mmol, 2.54 mL) drop wise at 0° C. The reaction mixture was then stirred at RT for 16 h. The reaction mixture was quenched with water (100 mL) and extracted with ethyl acetate (2×100 mL). The combined organic layer was dried over anhydrous sodium sulphate, concentrated under reduced pressure and purification by column chromatography silica (100-200, 20-30% ethyl acetate in pet ether) gave tert-butyl (3R)-4-[3-(2,6-dibenzyloxy-3-pyridyl)-1-methyl-indazol-6-yl]-3-(4-nitrobenzoyl)oxy-piperidine-1-carboxylate (1.6 g, 1.94 mmol, 60.25% yield) as a pale yellow gummy liquid. LCMS (ES⁺): m/z 770.2 [M+H]⁺.

### Step-6:

To a stirred solution of tert-butyl (3R)-4-[3-(2,6-dibenzyloxy-3-pyridyl)-1-methyl-indazol-6-yl]-3-(4-nitrobenzoyl)oxy-piperidine-1-carboxylate (1.6 g, 2.08 mmol) in water (4 mL), THE (12 mL) was added lithium hydroxide (149.32 mg, 6.24 mmol) at 0° C. and the reaction mixture was stirred for at 25° C. for 16 h. The reaction mixture was quenched with water (50 mL) and extracted with ethyl acetate (2×80 mL). The organic layer was washed with aqueous sodium bicarbonate solution (50 mL). The combined organic layer was dried over anhydrous sodium sulphate, concentrated under reduced pressure. The crude material was triturated with diethyl ether (100 mL) to afforded tert-butyl (3R,4S)-4-(3-(2,6-bis(benzyloxy)pyridin-3-yl)-1-methyl-1H-indazol-6-yl)-3-hydroxypiperidine-1-carboxylate (1.3 g, 1.80 mmol, 86.39% yield) as white solid. LCMS (ES⁺): m/z 621.32 [M+H]⁺.

### Step-7:

To a stirred solution of tert-butyl (3R,4S)-4-(3-(2,6-bis(benzyloxy)pyridin-3-yl)-1-methyl-1H-indazol-6-yl)-3-hydroxypiperidine-1-carboxylate (0.1 g, 0.161 mmol) in THE (3 mL), EtOAc (3 mL) and ethanol (1.5 mL) was added palladium 10% on carbon (0.1 g, 0.939 mmol). The reaction mixture was stirred at RT for 16 h under hydrogen bladder pressure. The reaction mixture was filtered through a celite pad and washed with DCM (50 mL). The organic layer was collected and evaporated under reduced pressure. The crude material was triturated with diethyl ether (100 mL) to afforded tert-butyl (3R,4S)-4-(3-(2,6-dioxopiperidin-3-yl)-1-methyl-1H-indazol-6-yl)-3-hydroxypiperidine-1-carboxylate (35 mg, 78.79 μmol, 48.91% yield) as white solid. LCMS (ES⁺): m/z 443.33 [M+H]⁺.

### Step-8:

To a stirred solution of tert-butyl (3R,4S)-4-(3-(2,6-dioxopiperidin-3-yl)-1-methyl-1H-indazol-6-yl)-3-hydroxypiperidine-1-carboxylate (0.05 g, 0.112 mmol) in DCM (15 mL) was 4.0 M HCl in dioxane (4 M, 0.5 ml, 333.33 μL) at 0° C. and the reaction mixture was stirred at RT for 3 h. The reaction mixture was evaporated under reduced pressure and the crude material was triturated in diethyl ether (15 mL) and pentane (10 mL) to afford 3-[1-methyl-6-[(3R,4S)-3-hydroxy-4-piperidyl]indazol-3-yl]piperidine-2,6-dione (38 mg, 99.87 mol, 88.39% yield, HCl salt) as an off white solid. Product was a mixture of the R,S and S,R enantiomers, and the stereochemistry was arbitrarily assigned. LCMS (ES⁺): m/z 343.33 [M+H]⁺. ¹H NMR (400 MHz, DMSO-d₆): δ 10.85 (s, 1H), 8.93 (d, J=10.4 Hz, 1H), 8.33 (s, 1H), 7.64 (d, J=8.4 Hz, 1H), 7.40 (s, 1H), 7.09 (d, J=8.4 Hz, 1H), 5.40 (br s, 1H), 4.36-4.32 (m, 1H), 4.11 (s, 1H), 3.98 (s, 3H), 3.48-3.38 (m, 1H), 3.21-3.20 (m, 2H), 3.13-3.03 (m, 2H), 2.73-2.58 (m, 2H), 2.50-2.36 (m, 2H), 2.19-2.16 (m, 1H), 1.82-1.79 (m, 1H).

### Synthesis of 3-[1-methyl-6-[(3R,4R)-3-hydroxy-4-piperidyl]indazol-3-yl]piperidine-2,6-dione

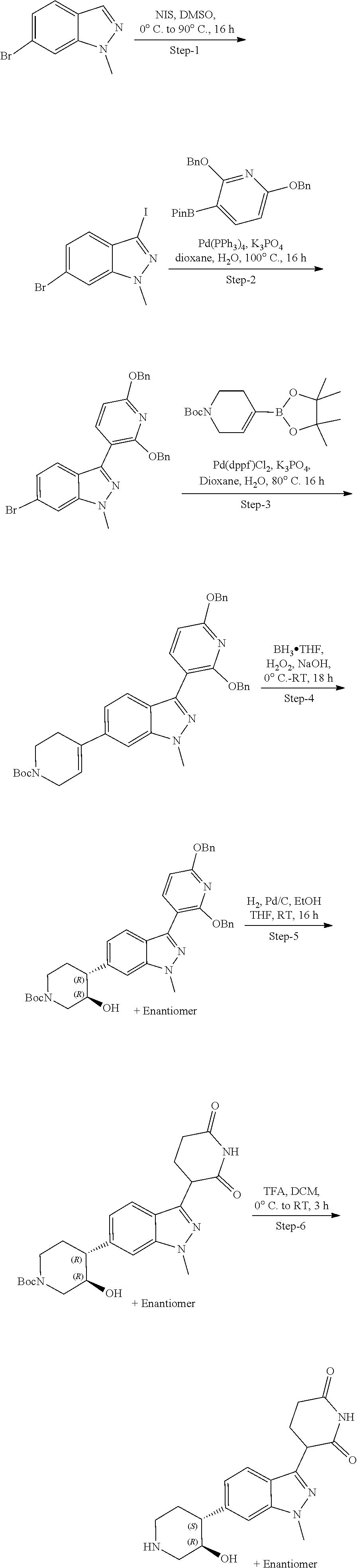

### Step-1:

To a stirred solution of 6-bromo-1-methyl-indazole (20 g, 94.76 mmol) in DMSO (200 mL) was added N-iodosuccinimide (25.58 g, 113.71 mmol) portion wise at 0° C. The reaction mixture was stirred at 90° C. for 16 h. After completion, the reaction mixture was quenched with ice cold saturated sodium thiosulfate (200 mL) solution. The solid precipitate in the reaction mixture was filtered under reduced pressure and dried to afford 6-bromo-3-iodo-1-methyl-indazole (30 g, 58.56 mmol, 61.79% yield) as a yellow solid. LCMS (ES⁺): m/z 337.15 [M+H]⁺.

### Step-2:

To a stirred solution of 6-bromo-3-iodo-1-methyl-indazole (15 g, 44.52 mmol) in dioxane (320 mL) and water (80 mL) were added 2,6-dibenzyloxy-3-(4,4,5,5-tetramethyl-1,3,2-dioxaborolan-2-yl)pyridine (18.58 g, 44.52 mmol) and tetrakis(triphenylphosphine)palladium(0) (5.14 g, 4.45 mmol) at RT under nitrogen gas. The reaction mixture was degassed with argon for 10 minutes. After degassing, potassium phosphate tribasic was added (28.35 g, 133.55 mmol) and the reaction kept on heating at 100° C. for 16 h. After completion of the reaction, the reaction mixture was cooled to RT and evaporated under reduced pressure. The obtained crude was diluted with water (50 mL) and extracted with ethyl acetate (2×100 mL). The combined organic layer was washed with brine (50 mL) and dried over anhydrous sodium sulfate and evaporated under reduced pressure to obtain crude. The crude was purified by column chromatography over silica gel (230-400 mesh, 0-30% EtOAc in pet-ether as eluent) to afford 6-bromo-3-(2,6-dibenzyloxy-3-pyridyl)-1-methyl-indazole (7 g, 11.89 mmol, 26.71% yield) as yellow solid. LCMS (ES⁺): m/z 502.19 [M+H]⁺.

### Step-3:

To a stirred solution of 6-bromo-3-(2,6-dibenzyloxy-3-pyridyl)-1-methyl-indazole (4.5 g, 8.99 mmol) in dioxane (40 mL) and water (10 mL) was added tert-butyl 4-(4,4,5,5-tetramethyl-1,3,2-dioxaborolan-2-yl)-3,6-dihydro-2H-pyridine-1-carboxylate (4.17 g, 13.49 mmol) portion wise. Potassium phosphate tribasic anhydrous was added (1.91 g, 8.99 mmol) at RT under nitrogen gas. The reaction mixture was degassed with argon for 10 minutes. After degassing PdCl₂(dppf)CH₂Cl₂ (734.41 mg, 899.31 μmol) was added, and the reaction mixture was stirred at 80° C. for 16 h. After consumption of starting material, the reaction mixture was concentrated under reduced pressure. The reaction mixture was diluted with water (50 mL) and extracted with ethyl acetate (2×100 mL). The combined organic layers were dried over anhydrous sodium sulphate and concentrated under reduced pressure to afford crude. The crude compound was purified by column chromatography (silica gel mesh 100-200,20-30% ethyl acetate in pet ether) to afford tert-butyl 4-[3-(2,6-dibenzyloxy-3-pyridyl)-1-methyl-indazol-6-yl]-3,6-dihydro-2H-pyridine-1-carboxylate (4.5 g, 4.89 mmol, 54.35% yield) as a brown liquid. LCMS (ES⁺): m/z 603.43 [M+H]⁺.

### Step-4:

To a stirred solution of tert-butyl 4-[3-(2,6-dibenzyloxy-3-pyridyl)-1-methyl-indazol-6-yl]-3,6-dihydro-2H-pyridine-1-carboxylate (4.50 g, 7.47 mmol) in THF (40 mL) was added borane tetrahydrofuran complex solution 1.0 M in THE (1 M, 18.67 mL) dropwise at 0° C. and the reaction mixture was stirred at RT for 1 h. After that the reaction mixture was cooled to 0° C. and quenched with hydrogen peroxide 35% (1.02 g, 29.86 mmol, 923.36 μL) followed by sodium hydroxide (1 M, 14.93 mL) and again stirred at RT for 16 h. After consumption of starting material, the reaction mixture was quenched with aq. saturated sodium sulphite (50 mL) solution and extracted with ethyl acetate (2×100 mL). The combined organic layers were dried over anhydrous Na₂SO₄ and concentrated under reduced pressure to obtain crude. The crude compound was purified by column chromatography (100-200 mesh silica gel, 10-20% ethyl acetate in pet ether) to afford desired product tert-butyl (3R,4R)-4-[3-(2,6-dibenzyloxy-3-pyridyl)-1-methyl-indazol-6-yl]-3-hydroxy-piperidine-1-carboxylate (3.2 g, 4.02 mmol, 53.86% yield) as a sticky colourless liquid. LCMS (ES⁺): m/z 621.49 [M+H]⁺.

### Step-5:

To a stirred solution of tert-butyl (3R,4R)-4-[3-(2,6-dibenzyloxy-3-pyridyl)-1-methyl-indazol-6-yl]-3-hydroxy-piperidine-1-carboxylate (3.2 g, 5.16 mmol) in THE (30 mL) and ethanol (30 mL) was added palladium 10% on carbon (3.29 g, 30.93 mmol). The reaction mixture was stirred at RT for 16 h under hydrogen bladder pressure. After completion of the reaction, the reaction mixture was filtered through celite and washed with ethyl acetate (50 mL). The organic layer was collected and evaporated under reduced pressure to get crude product. The resulting crude was triturated with diethyl ether (30 mL) to afford tert-butyl (3R,4R)-4-[3-(2,6-dioxo-3-piperidyl)-1-methyl-indazol-6-yl]-3-hydroxy-piperidine-1-carboxylate (2.5 g, 5.62 mmol, 108.93% yield) as a yellow solid. ¹H NMR (400 MHz, DMSO-d₆): δ 10.78 (s, 1H), 7.58 (d, J=8.4 Hz, 1H), 7.46 (s, 1H), 7.03 (d, J=8.0 Hz, 1H), 4.83 (d, J=6.8 Hz, 1H), 4.39-4.28 (m, 1H), 4.17 (bs, 2H), 3.96 (s, 3H), 3.52 (bs, 1H), 2.86-2.53 (bs, 4H), 2.88-2.68 (m, 1H), 2.21-2.09 (m, 1H), 1.82-1.08 (m, 2H), 1.43 (s. 9H). LCMS (ES⁺): m/z 443.46 [M+H]⁺

### Step-6:

A stirring solution of tert-butyl (3R,4R)-4-[3-(2,6-dioxo-3-piperidyl)-1-methyl-indazol-6-yl]-3-hydroxy-piperidine-1-carboxylate (0.80 g, 1.81 mmol) in DCM (10 mL) was cooled to 0° C. TFA (618.42 mg, 5.42 mmol, 417.85 μL) was added dropwise and the reaction mixture stirred at 25° C. for 3 h. After completion, the reaction mixture was evaporated under reduced pressure to obtain crude compound which was triturated with diethyl ether to afford 3-[1-methyl-6-[(3R,4R)-3-hydroxy-4-piperidyl]indazol-3-yl]piperidine-2,6-dione (700 mg, 1.53 mmol, 84.49% yield, TFA salt) as a white solid. Product was a mixture of the R,R and S,S enantiomer, and the stereochemistry was arbitrarily assigned. ¹H NMR (400 MHz, DMSO-d₆): δ 10.83 (s, 1H), 8.74-8.71 (bs, 1H), 8.56-8.50 (bs, 1H), 7.58 (d, J=8.4 Hz, 1H), 7.37 (s, 1H), 7.00 (d, J=8.0 Hz, 1H), 5.42 (bs, 1H), 4.38-4.31 (m, 1H), 3.97 (s, 3H), 3.82 (bs, 2H), 3.48-3.22 (bs, 2H), 3.07-2.91 (m, 1H), 2.82-2.54 (m, 3H), 2.42-2.39 (m, 1H), 2.23-2.11 (m, 1H), 1.92 (bs, 2H). LCMS (ES⁺): m/z 343.37 [M+H]⁺

### Synthesis of 1-(6-((3R,4S)-3-hydroxypiperidin-4-yl)-1-methyl-1H-indazol-3-yl)dihydropyrimidine-2,4(1H,3H)-dione

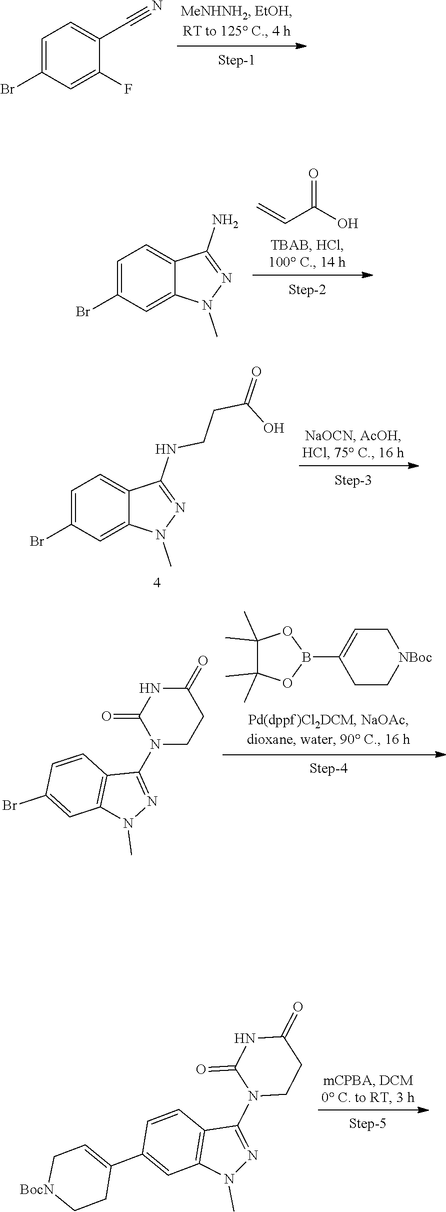

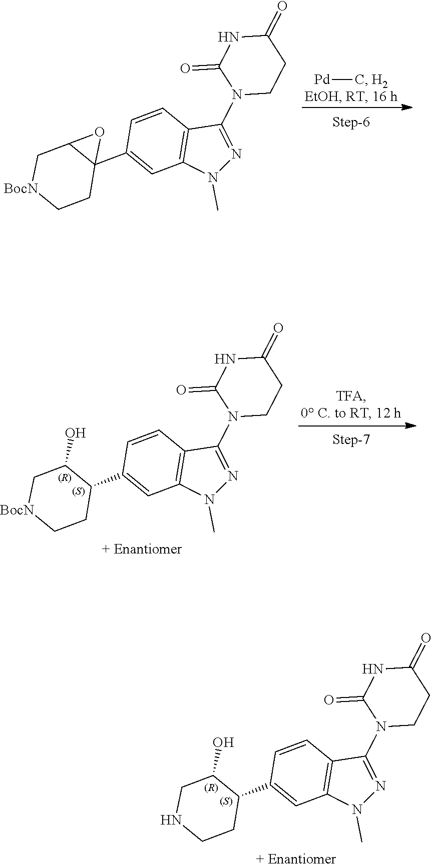

### Step-1:

To a stirred solution of 4-bromo-2-fluoro-benzonitrile (25.0 g, 125 mmol) in EtOH (500 mL) was added methyl hydrazine, 85% in water (65 mL) dropwise at −28° C. The reaction mixture was further stirred at 125° C. in 1 L autoclaved for 4 h. Upon completion of the reaction, the reaction mixture was cooled to RT and diluted with cold water (2 L) and stirred for 30 min. The precipitate was filtered and washed with cold water (1 L) and dried well to afford 6-bromo-1-methyl-1H-indazol-3-amine (21.0 g, 67.71% yield) as an off-white solid. LCMS (ES⁻): m/z 226.9 [M−H]⁻

### Step-2:

To a stirred solution of 6-bromo-1-methyl-1H-indazol-3-amine (120.0 g, 530.8 mmol) in aqueous HCl (2M) (1.2 L) was added tetrabutyl ammonium bromide (17.11 g, 53.08 mmol) at 28° C. The reaction mixture was stirred at 55° C. and acrylic acid (45.9 g, 636.96 mmol, 43.67 mL) was added dropwise at 55° C. The reaction mixture was stirred at 100° C. for 12 h, while monitoring progress by LCMS and TLC. Upon completion, the reaction mixture was cooled to RT and diluted with cold water (2.5 L) and stirred for 30 min. The aqueous layer was basified by using aqueous sodium bicarbonate solution (1 L) and stirred for 1 h. The precipitated solid was filtered and washed with cold water (1 L) and dried well to afford 3-((6-bromo-1-methyl-1H-indazol-3-yl)amino) propanoic acid (85.0 g, 48.28% yield) as an off white solid. LCMS (ES⁺): m/z 297.57 [M+H]⁺

### Step-3:

To a stirred solution of 3-((6-bromo-1-methyl-1H-indazol-3-yl)amino)propanoic acid (50.0 g, 167.71 mmol) in AcOH (700 mL) was added sodium cyanate (21.8 g, 335.42 mmol) at 28° C. The reaction mixture was stirred at 75° C. for 12 h. Aqueous HCl (4 M, 500 mL) was added dropwise and the reaction was stirred at 75° C. for 4 h. Upon completion, the reaction mixture was cooled to 0° C. and stirred for 1 h. The precipitated solid was filtered and dried well to afford 1-(6-bromo-1-methyl-1H-indazol-3-yl)dihydropyrimidine-2,4(1H,3H)-dione (26.0 g, 47.5% yield) as an off white solid. LCMS (ES⁺): m/z 323.32 [M+H]⁺

### Step-4:

To a stirred solution of 1-(6-bromo-1-methyl-1H-indazol-3-yl)dihydropyrimidine-2,4(1H,3H)-dione (5) (10.0 g, 30.95 mmol) tert-butyl 4-(4,4,5,5-tetramethyl-1,3,2-dioxaborolan-2-yl)-3,6-dihydropyridine-1(2H)-carboxylate (14.35 g, 46.42 mmol) in 1,4 dioxane (196 mL) and water (49 mL) was added. Sodium acetate, anhydrous (6.35 g, 77.36 mmol) was added portion-wise at 28° C. The reaction mixture was degassed under argon atmosphere for 10 min. and Pd(dppf)Cl₂ DCM (1.26 g, 1.55 mmol) was added and again degassed under argon atmosphere for 10 min. The reaction mixture was stirred at 90° C. for 16 h. After completion of the reaction, the reaction mixture was cooled to room temperature and filtered through celite. The celite bed was washed with ethyl acetate (300 mL). The filtrate was evaporated to obtain crude which was diluted with water (200 mL) and extracted with ethyl acetate (3×100 mL). The organic layers were collected and dried over anhydrous sodium sulfate and evaporated under reduced pressure to obtain crude. The crude was purified by silica gel column chromatography 100-200 mesh (0-90% ethyl acetate in pet ether as an eluent) to afford tert-butyl 4-(3-(2,4-dioxotetrahydropyrimidin-1(2H)-yl)-1-methyl-1H-indazol-6-yl)-3,6-dihydropyridine-1(2H)-carboxylate (10.0 g, 58.69% yield) as yellow solid. LCMS (ES⁺): m/z 426.39 [M+H]⁺

### Step-5:

To a stirred solution of tert-butyl 4-(3-(2,4-dioxotetrahydropyrimidin-1(2H)-yl)-1-methyl-1H-indazol-6-yl)-3,6-dihydropyridine-1(2H)-carboxylate (2.0 g, 4.7 mmol) in DCM (80 mL) was added m-CPBA (1.62 g, 9.40 mmol) at 0° C. The reaction mixture was stirred at 0° C. for 3 h. After completion, the reaction mixture was diluted with aqueous saturated sodium bicarbonate solution (60 mL) and extracted with DCM (2×30 mL). The combined organic layers were washed with brine (30 mL), collected, and dried over anhydrous sodium sulphate and evaporated under reduced pressure to obtain crude. The crude was triturated with diethyl ether (60 mL) to afford tert-butyl 6-(3-(2,4-dioxotetrahydropyrimidin-1(2H)-yl)-1-methyl-1H-indazol-6-yl)-7-oxa-3-azabicyclo[4.1.0]heptane-3-carboxylate (1.08 g, 39.39% yield) as an off-white solid. LCMS (ES⁺): m/z 442.43 [M+H]⁺

### Step-6:

To a stirred solution of tert-butyl 6-(3-(2,4-dioxotetrahydropyrimidin-1(2H)-yl)-1-methyl-1H-indazol-6-yl)-7-oxa-3-azabicyclo[4.1.0]heptane-3-carboxylate (5.0 g, 11.33 mmol) in ethanol (200 mL) was added 10% palladium on carbon 50% wet basis (5.0 g, 11.33 mmol) and the reaction mixture was stirred under hydrogen atmosphere for 16 h. Upon completion, the reaction mixture was filtered through celite pad and washed with 10% MeOH in DCM (100 mL). The filtrate was concentrated under reduced pressure to get crude product which was triturated with diethyl ether (25 mL) and pentane (25 mL) to afford tert-butyl (3R,4S)-4-(3-(2,4-dioxotetrahydropyrimidin-1(2H)-yl)-1-methyl-1H-indazol-6-yl)-3-hydroxypiperidine-1-carboxylate (3.9 g, 66.08% yield) as an off-white solid. LCMS (ES⁻): m/z 442.14 [M−H]⁻ Step-7: To a stirred solution of tert-butyl (3R,4S)-4-(3-(2,4-dioxotetrahydropyrimidin-1(2H)-yl)-1-methyl-1H-indazol-6-yl)-3-hydroxypiperidine-1-carboxylate (2.0 g, 4.51 mmol) in DCM (30 mL) was added trifluoracetic acid (1.74 g, 22.55 mmol) at 0° C. The reaction mixture was stirred at 28° C. for 12 h. Upon completion of the reaction, the reaction mixture was concentrated under reduced pressure to get crude product. The crude compound was triturated with diethyl ether (30 mL) and the precipitated solid was filtered and dried to afford 1-(6-((3R,4S)-3-hydroxypiperidin-4-yl)-1-methyl-1H-indazol-3-yl)dihydropyrimidine-2,4(1H,3H)-dione (1.91 g, 49.47% yield, TFA salt) as an off-white solid. Product was a mixture of the R,S and S,R enantiomers, and the stereochemistry was arbitrarily assigned. ¹H NMR (400 MHz, DMSO-d₆): δ 11.55 (s, 1H), 8.72 (bs, 1H), 8.30 (bs, 1H), 7.58 (d, J=4.4 Hz, 1H), 7.40 (s, 1H), 7.08 (d, J=8.4 Hz, 1H), 5.41 (bs, 1H), 4.13 (bs, 1H), 3.99-3.88 (m, 5H), 3.38-3.06 (m, 5H), 2.74 (t, J=7.2 Hz, 2H), 2.45-2.31 (m, 1H), 1.84 (d, J=8.8 Hz, 1H). LCMS (ES⁺): m/z 343.92 [M+H]⁺

### Synthesis of 1-[1-methyl-6-[(3R,4S)-3-methoxy-4-piperidyl]indazol-3-yl]hexahydropyrimidine-2,4-dione

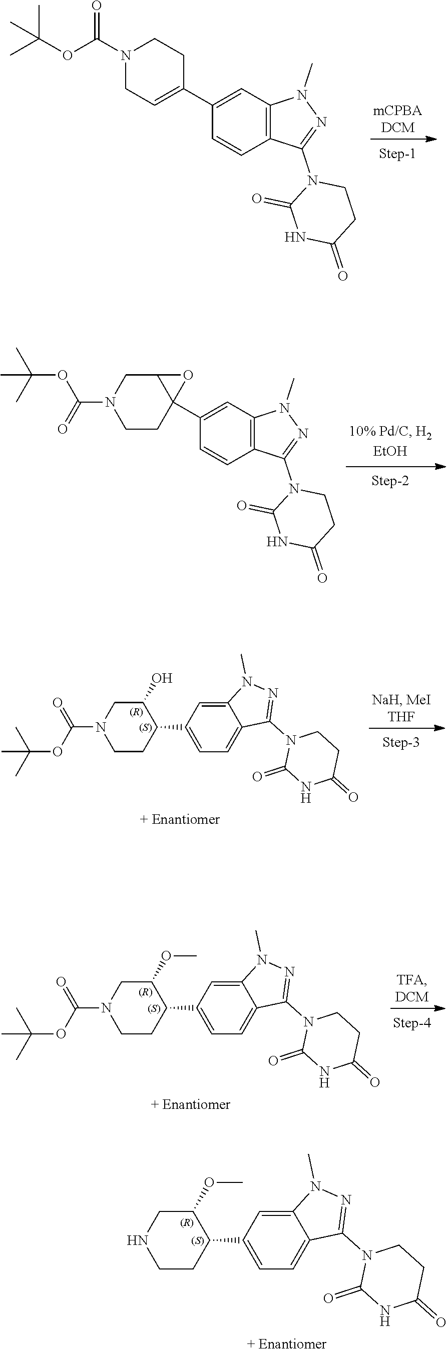

### Step-1:

To a stirred solution of tert-butyl 4-[3-(2,4-dioxohexahydropyrimidin-1-yl)-1-methyl-indazol-6-yl]-3,6-dihydro-2H-pyridine-1-carboxylate (2 g, 4.70 mmol) in DCM (20 mL) was added meta-chloroperoxybenzoic acid (1.22 g, 7.05 mmol) at 0° C. and stirred at 0° C. for 3 h. The reaction mixture was diluted with DCM (50 vol), washed with saturated NaHCO₃ bisulfite (30 vol), saturated potassium carbonate (30 vol), dried over anhydrous sodium sulphate and concentrated under reduced pressure to give crude material. The crude was material was purified by reverse phase column chromatography (Reveleris C18, 40 g, A: Ammonium acetate in Water, B:ACN, 0-70% gradient) to afford tert-butyl 6-[3-(2,4-dioxohexahydropyrimidin-1-yl)-1-methyl-indazol-6-yl]-7-oxa-3-azabicyclo[4.1.0]heptane-3-carboxylate (1 g, 1.90 mmol, 40.41% yield) as a brown solid. LCMS (ES⁺): m/z 442.26 [M+H]⁺.

### Step-2:

The stirred solution of tert-butyl 6-[3-(2,4-dioxohexahydropyrimidin-1-yl)-1-methyl-indazol-6-yl]-7-oxa-3-azabicyclo[4.1.0]heptane-3-carboxylate (1 g, 2.27 mmol) in ethanol (10 mL) was degassed with nitrogen for 2 min. To the mixture was added palladium, 10% on carbon, 50% wet (500.00 mg, 4.70 mmol) and stirred at RT under a hydrogen bladder for 12 h. The mixture was diluted with 1:1 MeOH:DCM (100 vol), filtered through celite and the filtrate was concentrated under reduced pressure to get crude. The crude was purified by reverse-phase column chromatography (Reveleris C18, 40 g, 0-50% A: 0.1% Ammonium acetate in water, B:ACN) to afford tert-butyl (3R,4S)-4-[3-(2,4-dioxohexahydropyrimidin-1-yl)-1-methyl-indazol-6-yl]-3-hydroxy-piperidine-1-carboxylate (0.55 g, 1.19 mmol, 52.59% yield) as off white solid. LCMS (ES⁺): m/z 444.62 [M+H]⁺.

### Step-3:

To a stirred solution of tert-butyl (3R,4S)-4-[3-(2,4-dioxohexahydropyrimidin-1-yl)-1-methyl-indazol-6-yl]-3-hydroxy-piperidine-1-carboxylate (300 mg, 676.45 μmol) in THE (5 mL) was added sodium hydride 60% dispersion in mineral oil (81.17 mg, 2.03 mmol), then the reaction was stirred for 1 h at RT, again the reaction mixture was cooled to 0° C. then iodomethane (211.23 mg, 1.49 mmol, 92.65 μL) was added into the reaction mixture and continued to stir for 4 h at RT. When SM was consumed confirming by TLC, then the reaction mixture was quenched with saturated ammonium chloride solution and extracted with ethyl acetate. The organic layer was washed further with brine solution, dried over sodium sulfate, concentrated under reduced pressured and purified using column chromatography using 230-400 silica mesh (5-10% MeOH-DCM) to give tert-butyl (3R,4S)-4-[3-(2,4-dioxohexahydropyrimidin-1-yl)-1-methyl-indazol-6-yl]-3-methoxy-piperidine-1-carboxylate (40 mg, 76.94 μmol, 11.37% yield) as a colorless liquid. LCMS (ES⁺): m/z 480.55 [M+Na]⁺.

### Step-4:

To a stirred solution of tert-butyl (3R,4S)-4-[3-(2,4-dioxohexahydropyrimidin-1-yl)-1-methyl-indazol-6-yl]-3-methoxy-piperidine-1-carboxylate (40 mg, 87.43 μmol) in DCM (3 mL) was added TFA (0.5 mL) at 0° C. and the resultant suspension was stirred for 2 hr. Upon completion of the reaction, the reaction mixture was concentrated under reduced pressure to give the crude product, which was triturated with diethyl ether (5 mL×2) to afford 1-[1-methyl-6-[(3R,4S)-3-methoxy-4-piperidyl]indazol-3-yl]hexahydropyrimidine-2,4-dione (40 mg, 72.12 μmol, 82.49% yield, TFA salt). Product was a mixture of the R,S and S,R enantiomers, and the stereochemistry was arbitrarily assigned. LCMS (ES⁺): m/z 358.17 [M+H]⁺.

### Synthesis of tert-butyl 1-[1-methyl-6-[(1R,2R,4R)-4-amino-2-hydroxy-cyclohexyl]indazol-3-yl]hexahydropyrimidine-2,4-dione

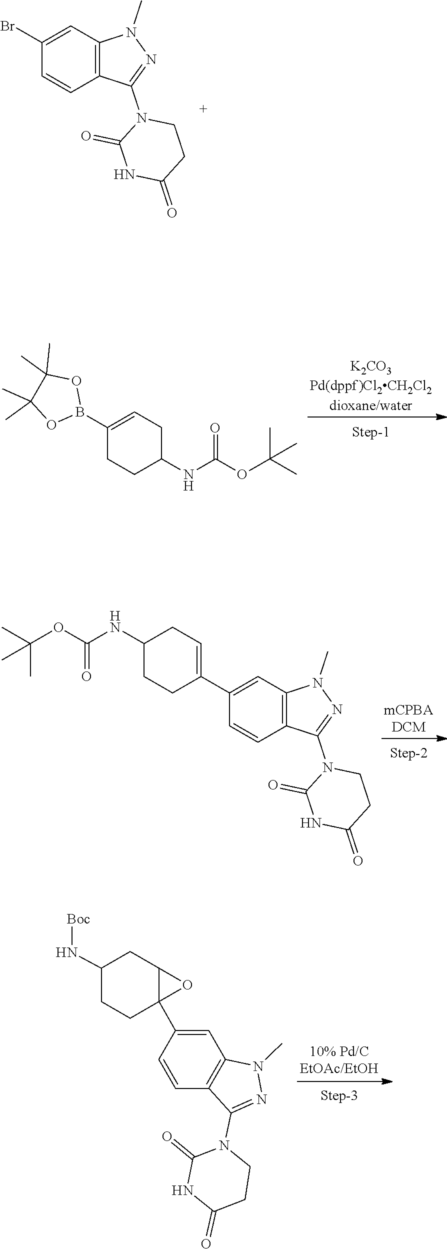

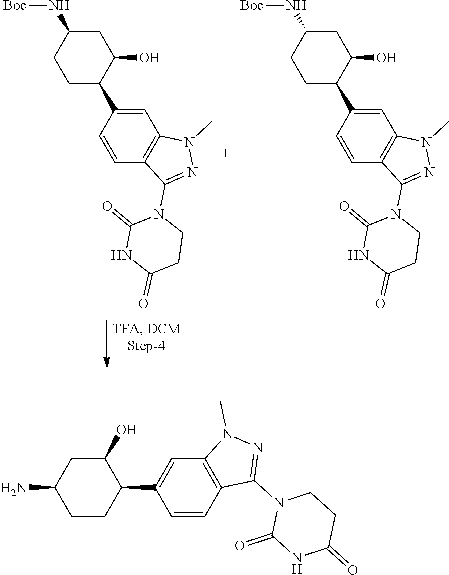

### Step-1:

To a solution of 1-(6-bromo-1-methyl-indazol-3-yl)hexahydropyrimidine-2,4-dione (1 g, 3.09 mmol) and tert-butyl N-[4-(4,4,5,5-tetramethyl-1,3,2-dioxaborolan-2-yl)cyclohex-3-en-1-yl]carbamate (1.00 g, 3.09 mmol) in dioxane (8 mL) and water (2 mL) was added potassium carbonate (granular) (1.28 g, 9.27 mmol) at RT. The reaction mixture was degassed with argon gas for 10 minutes and Pd(dppf)Cl₂ (22.61 mg, 30.90 μmol) was added. The reaction mixture was degassed with argon for additional 5 minutes and it was stirred at 80° C. for 16 hr. Subsequently, the reaction mixture was quenched with water (50 mL) and extracted with ethyl acetate (3×50 mL). The combined organic layer was washed with brine solution (50 mL), dried over anhydrous sodium sulphate and concentrated under reduced pressure to give material, which was purified by column chromatography using Davisil silica and 5% EA: pet ether as eluent to afford tert-butyl N-[4-[3-(2,4-dioxohexahydropyrimidin-1-yl)-1-methyl-indazol-6-yl]cyclohex-3-en-1-yl]carbamate (0.9 g, 1.99 mmol, 64.35% yield) as white solid. LCMS (ES⁺): m/z 440.40 [M+H]⁺.

### Step-2:

To a solution of tert-butyl N-[4-[3-(2,4-dioxohexahydropyrimidin-1-yl)-1-methyl-indazol-6-yl]cyclohex-3-en-1-yl]carbamate (0.1 g, 227.53 μmol,) in DCM (2 mL) was added mCPBA (78.53 mg, 455.06 μmol. The reaction mixture was stirred at 0° C. for 2 hr. Subsequently, the reaction mixture was quenched with water (30 mL) and extracted with ethyl acetate (3×20 mL). The combined organic layers was washed with brine (50 mL), dried over anhydrous sodium sulphate, concentrated under reduced pressure and purification by column chromatography (Davisil silica, 50% EA: pet ether as eluent) gave tert-butyl N-[6-[3-(2,4-dioxohexahydropyrimidin-1-yl)-1-methyl-indazol-6-yl]-7-oxabicyclo[4.1.0]heptan-3-yl]carbamate (0.06 g, 84.16 μmol, 36.99% yield) as white solid. LCMS (ES⁺): m/z 456.39 [M+H]⁺.

### Step-3:

To a solution of tert-butyl N-[6-[3-(2,4-dioxohexahydropyrimidin-1-yl)-1-methyl-indazol-6-yl]-7-oxabicyclo[4.1.0]heptan-3-yl]carbamate (5.4 g, 11.85 mmol) in ethyl acetate (50 mL) at room temperature was added 10% Pd/C, 50% wt. basis (5.4 g, 131.72 μmol). The reaction mixture was stirred at 25° C. for 2 h under a hydrogen atmosphere. The reaction mixture was filtered through a celite bed and concentrated under reduced pressure to give crude material, which was purified by reverse phase using 0.1% formic acid in water and ACN to afford tert-butyl N-[(1R,3R,4R)-4-[3-(2,4-dioxohexahydropyrimidin-1-yl)-1-methyl-indazol-6-yl]-3-hydroxy-cyclohexyl]carbamate (Peak 1, 1 g, 1.69 mmol, 14.26% yield) and tert-butyl N-[(1S,3R,4R)-4-[3-(2,4-dioxohexahydropyrimidin-1-yl)-1-methyl-indazol-6-yl]-3-hydroxy-cyclohexyl]carbamate (Peak 2, 0.5 g, 927.28 μmol, 7.82% yield) as a white solid. Peak 1: LCMS (ES⁺): m/z 458.35 [M+H]⁺. Peak 2: LCMS (ES⁺): m/z 458.20 [M+H]⁺.

### Step-4:

To a stirred solution of tert-butyl N-[(1R,3R,4R)-4-[3-(2,4-dioxohexahydropyrimidin-1-yl)-1-methyl-indazol-6-yl]-3-hydroxy-cyclohexyl]carbamate (550 mg, 1.20 mmol) in DCM (10 mL) was added TFA (1.71 g, 15.03 mmol, 1.16 mL) at 0° C. under a nitrogen atmosphere. The reaction mixture was warm to RT and stirred for 16 h. The reaction mixture was concentrated in vacuo to give crude material, which was triturated with diethyl ether (40 mL) to afford 1-[1-methyl-6-[(1R,2R,4R)-4-amino-2-hydroxy-cyclohexyl]indazol-3-yl]hexahydropyrimidine-2,4-dione (600 mg, 1.13 mmol, 94.37% yield, TFA salt) as an off-white solid. Stereochemistry of the product was arbitrarily assigned. LCMS (ES⁺): m/z 358.14 [M+H]⁺.

### Synthesis of 1-(5-fluoro-1-methyl-6-(piperidin-4-yl)-1H-indazol-3-yl)dihydropyrimidine-2,4(1H,3H)-dione

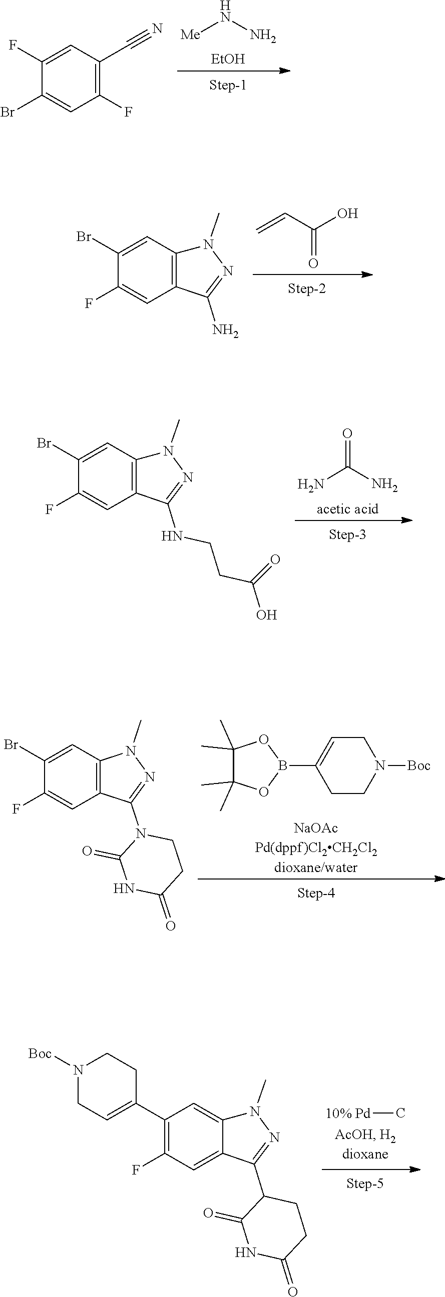

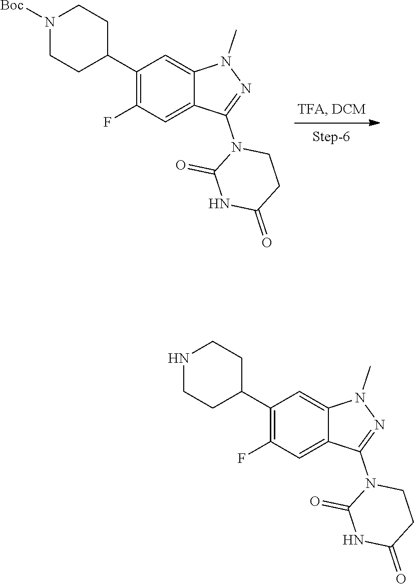

### Step-1:

To a solution of 4-bromo-2,5-difluoro-benzonitrile (50 g, 229.36 mmol) in ethanol (10 mL) was added methyl hydrazine (12.68 g, 275.23 mmol) at room temperature under argon atmosphere. The resulting mixture was heated at 85° C. for 12 h. After consumption of the starting material, the reaction mixture was poured into ice cold water (500 mL), and the precipitate was filtered and dried under vacuum to give the crude compound, which was triturated with n-Pentane to afford 6-bromo-5-fluoro-1-methyl-indazol-3-amine (44 g, 167.66 mmol, 73.10% yield) as an off-white solid. LCMS (ES⁺): m/z 244.21 [M+H]⁺.

### Step-2:

To a stirred suspension of 6-bromo-5-fluoro-1-methyl-indazol-3-amine (25 g, 102.43 mmol) in hydrochloric acid, 36% w/w aq. soln. (2 M, 256.08 mL) was added tetrabutylammonium bromide (3.30 g, 10.24 mmol) at 0° C. The resulting mixture was stirred at 60° C. for 16 h. To this mixture was added acrylic acid (9.60 g, 133.16 mmol, 9.13 mL) at this temperature over a period of 10 min and the reaction was stirred for another 16 h. After complete consumption of starting material, the reaction mixture was neutralized to pH 6-7 and the precipitate was filtered and dried under vacuum to afford 3-[(6-bromo-5-fluoro-1-methyl-indazol-3-yl) amino] propanoic acid (24 g, 50.11 mmol, 48.92% yield) as an off-white solid.

### Step-3:

To the stirred solution of 3-[(6-bromo-5-fluoro-1-methyl-indazol-3-yl) amino]propanoic acid (24 g, 75.92 mmol) in acetic acid (240 mL) was added sodium cyanate (9.87 g, 151.84 mmol) at room temperature. The resulting reaction mixture was heated at 75° C. for 12 h, then aqueous hydrogen chloride solution (4 M, 226.34 mL) was added to the reaction mixture at 75° C. over a period of 15 min and the reaction was stirred at the same temperature for 4 h. After complete consumption of starting material, the reaction mixture was cooled slowly to 0-5° C. with vigorous stirring. The precipitated solid was filtered to give the crude material, which was triturated with pet ether (100 mL) to give 1-(6-bromo-5-fluoro-1-methyl-indazol-3-yl) hexahydropyrimidine-2,4-dione (10 g, 27.54 mmol, 36.28% yield) as an off-white solid. LCMS (ES⁺): m/z 342.7 [M+H]⁺.

### Step-4:

To a stirred solution of 1-(6-bromo-5-fluoro-1-methyl-indazol-3-yl)hexahydropyrimidine-2,4-dione (500 mg, 1.47 mmol) in dioxane (10 mL) was added tert-butyl 4-(4,4,5,5-tetramethyl-1,3,2-dioxaborolan-2-yl)-3,6-dihydro-2H-pyridine-1-carboxylate 6 (679.81 mg, 2.20 mmol) and sodium acetate (360.71 mg, 4.40 mmol) at room temperature. The resulting mixture was degassed with argon for 20 minutes and PdCl₂ (dppf) (107.25 mg, 146.57 μmol) was added. The reaction mixture was heated at 100° C. and stirred for 16 h. After complete consumption of starting material, the reaction mixture was filtered through a pad of celite and washed with ethyl acetate. The filtrate was washed with water and brine solution, dried over anhydrous sodium sulfate, filtered and concentrated under reduced pressure to give crude material which was purified by column chromatography (Davisil silica) using 50-60% of EtOAc in Pet-Ether as eluent to afford tert-butyl 4-[3-(2,4-dioxohexahydropyrimidin-1-yl)-5-fluoro-1-methyl-indazol-6-yl]-3,6-dihydro-2H-pyridine-1-carboxylate 7 (300 mg, 608 μmol, 41.5% yield) as an off white solid. LCMS (ES⁺): m/z 444.53 [M+H]⁺.

### Step-5:

To a stirred solution of tert-butyl 4-[3-(2,4-dioxohexahydropyrimidin-1-yl)-5-fluoro-1-methyl-indazol-6-yl]-3,6-dihydro-2H-pyridine-1-carboxylate (1.0 g, 2.25 mmol) in ethyl acetate (10 mL) and THE (10 mL) was added PtO₂ (153.62 mg, 676.48 μmol) at room temperature. The resulting mixture was stirred vigorously under hydrogen atmosphere (balloon) at room temperatures for 16 h. After the complete consumption of starting material, the reaction mixture was filtered through a celite bed, and washed with ethyl acetate (100 mL). The filtrate was concentrated and dried under high vacuum to give the crude material which was triturated with diethyl ether (10 mL) to afford tert-butyl4-[3-(2,4-dioxohexahydropyrimidin-1-yl)-5-fluoro-1-methyl-indazol-6-yl] piperidine-1-carboxylate (800 mg, 1.59 mmol, 70.42% yield) as an off-white solid. LCMS (ES⁻): m/z 444.34 [M−H]⁻.

¹H NMR (400 MHz, DMSO-d₆): δ 10.53 (s, 1H), 7.59 (d, J=5.9 Hz, 1H), 7.37 (d, J=10.9 Hz, 1H), 4.13 (d, J=11.2 Hz, 1H), 3.99 (s, 1H), 3.90 (t, J=6.7 Hz, 1H), 3.06 (t, J=11.9 Hz, 1H), 2.87 (s, 1H), 2.75 (t, J=6.7 Hz, 1H), 1.81 (d, J=12.3 Hz, 1H), 1.64 (m, 1H), 1.43 (s, 1H).

### Step-6:

A stirred solution of tert-butyl 4-[3-(2,4-dioxohexahydropyrimidin-1-yl)-5-fluoro-1-methyl-indazol-6-yl] piperidine-1-carboxylate (50 mg, 112.24 μmol) in DCM (2.5 mL) was cooled to 0° C. and added TFA (12.80 mg, 112.24 μmol, 8.65 μL) over the period of 5 minutes and then stirred at room temperature for 4 h. After complete consumption of the starting material, the reaction mixture was concentrated under reduced pressure and co-distilled with toluene (10 mL) and triturated with diethyl ether (10 mL) to afford 1-[5-fluoro-1-methyl-6-(4-piperidyl) indazol-3-yl] hexahydropyrimidine-2,4-dione (40 mg, 81.47 μmol, 72.59% yield, TFA salt) as a yellow solid. LCMS (ES⁺): m/z 346.80 [M+H]⁺. ¹H NMR (400 MHz, DMSO-d₆): δ 10.55 (s, 1H), 8.64 (d, J=8.8 Hz, 1H), 8.32 (d, J=8.3 Hz, 1H), 7.45 (q, 1H), 4.01 (s, 1H), 3.90 (t, J=6.6 Hz, 1H), 3.15 (m, 1H), 2.75 (t, J=6.6 Hz, 1H), 1.95 (m, 1H).

### F. Synthesis of Representative Compounds

### Synthesis of 2-[4-(hydroxymethyl)cyclohexyl]-7-isopropoxy-N-[6-(trifluoromethyl)-2-pyridyl]imidazo[1,2-a]pyridine-6-carboxamide

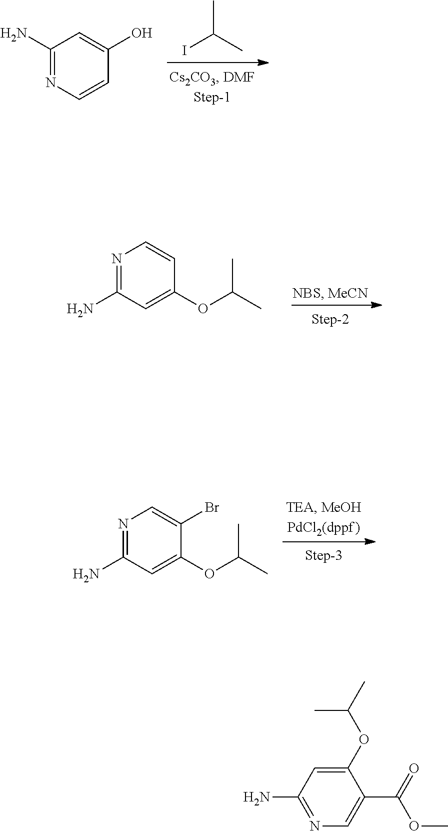

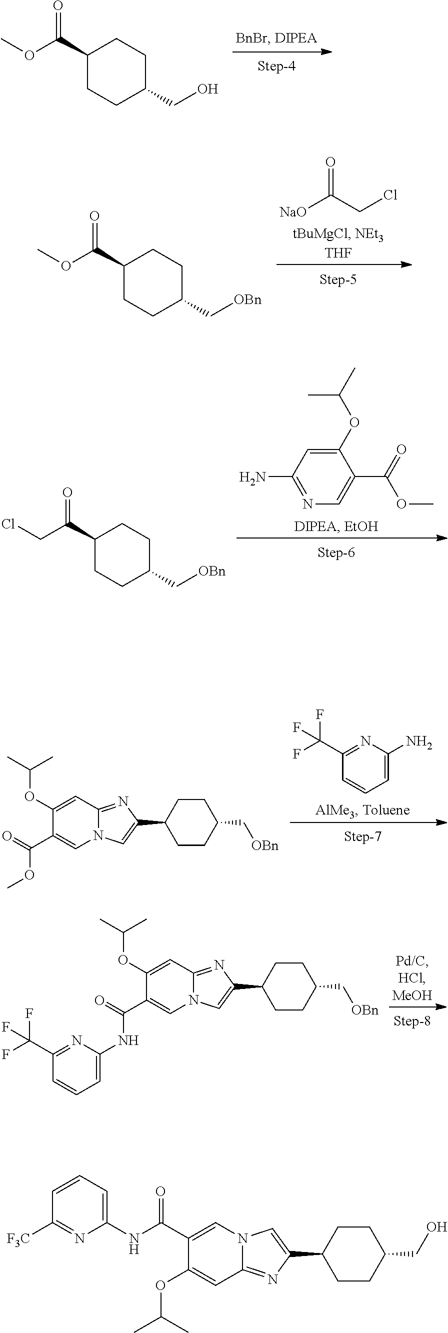

### Step-1:

To a stirred solution of 2-aminopyridin-4-ol (38 g, 345.10 mmol) in DMF (400 mL), cesium carbonate (134.93 g, 414.12 mmol) was added at 0° C. After 30 min, 2-iodopropane (50.17 g, 295.15 mmol, 29.51 mL) was added and the reaction mixture was stirred at room temperature for 4 h. After completion of the reaction as confirmed by TLC, the reaction mixture was poured into water (10 V), extracted with ethyl acetate (2×10 V), dried over Na₂SO₄ and concentrated under reduced pressure to afford 4-isopropoxypyridin-2-amine (34 g, 187.66 mmol, 54.38% yield). LCMS (ES⁺): m/z 153.38 [M+H]⁺

### Step-2:

A solution of 4-isopropoxypyridin-2-amine (34 g, 223.40 mmol) in acetonitrile (400 mL) was cooled to 0° C. and NBS (43.74 g, 245.74 mmol) was added after 30 min. The reaction mixture was stirred at room temperature for 6 h. Upon completion of the reaction as confirmed by TLC, the reaction mixture was poured into water (10 V), extracted with ethyl acetate (2×10 V), dried over Na₂SO₄ and concentrated under reduced pressure. The crude product was purified by column chromatography (silica gel, 70% EA/Pet Ether) to afford 5-bromo-4-isopropoxy-pyridin-2-amine (22 g, 91.39 mmol, 40.91% yield). LCMS (ES⁺): m/z 231.09 [M+H]⁺

### Step-3:

To a stirred solution of 5-bromo-4-isopropoxy-pyridin-2-amine (5 g, 21.64 mmol) in methanol (100 mL), TEA (10.95 g, 108.18 mmol, 15.08 mL) was added at room temperature and purged with argon gas for 30 min. This was followed by the addition of PdCl₂(dppf) (1.90 g, 2.60 mmol). The reaction was placed in the autoclave with 400 psi carbon monoxide pressure and heated at 100° C. for 48 h. After completion of the reaction, the reaction mixture was concentrated under reduced pressure. The crude product was purified by column chromatography using neutral alumina (50% Ethyl acetate/Pet Ether) to afford methyl 6-amino-4-isopropoxy-pyridine-3-carboxylate (3.25 g, 13.76 mmol, 63.59% yield). LCMS (ES⁺): m/z 211.24 [M+H]⁺

### Step-4:

In a sealed tube, a solution of methyl 4-(hydroxymethyl)cyclohexanecarboxylate (37.5 g, 217.74 mmol), DIPEA (61.22 g, 473.65 mmol, 82.50 mL) and bromomethylbenzene (64.80 g, 378.87 mmol, 45.00 mL) was stirred at 130° C. for 8 hours. After the reaction was complete, it was quenched with water and extracted with ethyl acetate. The organic layer was washed with brine, dried over anhydrous Na₂SO₄, and concentrated in vacuo to give the crude compound. It was then purified by column chromatogram (0 to 15% ethyl acetate in pet ether) to afford methyl 4-(benzyloxymethyl) cyclohexanecarboxylate (35 g, 133.41 mmol, 61.27% yield). ¹H NMR (400 MHz, CDCl₃) δ: 7.34 (m, 5H), 4.49 (s, 2H), 3.66 (s, 2H), 3.28 (d, J=6.36 Hz, 2H), 2.24 (m, 1H), 1.99 (t, J=6.42 Hz, 2H), 1.90 (q, J=5.18 Hz, 2H), 1.62 (m, 2H), 1.43 (m, 2H), 1.00 (m, 2H).

### Step-5:

To a stirred solution of methyl 4-(benzyloxymethyl)cyclohexanecarboxylate (40 g, 152.47 mmol) in THE (1.28 L) were added sodium chloroacetate, 98% (71.04 g, 609.89 mmol) and TEA (61.71 g, 609.89 mmol, 85.01 mL) at 25° C. The reaction mixture was cooled at −10 to −5° C., before 2.0 M tert-butyl magnesium chloride solution in THE (609.89 mmol) was slowly added at −10 to −5° C. The reaction mixture was stirred for 5 minutes at this temperature and then warmed up to 25° C. and stirred for an additional 5 hours. Upon completion of the reaction, the reaction was quenched with cold saturated ammonium chloride solution and extracted with ethyl acetate. The combined organic layers were washed with brine, dried over anhydrous Na₂SO₄, and concentrated in vacuo to afford the product 1-[4-(benzyloxymethyl)cyclohexyl]-2-chloro-ethanone (35 g, 124.65 mmol, 81.75% yield) as a yellow liquid. ¹H NMR (400 MHz, CDCl₃) δ: 7.34 (m, 5H). 4.49 (s, 2H), 4.32 (s, 1H), 4.16 (s, 2H), 3.29 (d, J=6.27 Hz, 2H), 2.61 (q, J=8.11 Hz, 1H), 1.93 (d, J=11.27 Hz, 3H), 1.41 (m, 1H), 1.64 (m, 2H), 1.05 (m, 2H).

### Step-6:

To a stirred solution of 1-[4-(benzyloxymethyl)cyclohexyl]-2-chloro-ethanone (11.22 g, 39.96 mmol) in ethanol (8 mL) was added methyl 6-amino-4-isopropoxy-pyridine-3-carboxylate (6 g, 28.54 mmol) and DIPEA (55.33 g, 428.10 mmol, 74.57 mL) at room temperature and stirred at 100° C. in a sealed tube for 4 h. Upon completion of the reaction, the reaction mixture was cooled to room temperature and concentrated in vacuo. The residue was added to 10 V water, extracted with ethyl acetate (2×10 V), dried over anhydrous Na₂SO₄ and concentrated under reduced pressure. The crude product was purified by column chromatography (neutral alumina, 30% EA/Pet Ether) to afford methyl 2-[4-(benzyloxymethyl)cyclohexyl]-7-isopropoxy-imidazo[1,2-a]pyridine-6-carboxylate (7 g, 14.43 mmol, 50.57% yield). LCMS (ES⁺): m/z 437.52 [M+H]⁺

### Step-7:

To a stirred solution of methyl 2-[4-(benzyloxymethyl)cyclohexyl]-7-isopropoxy-imidazo[1,2-a]pyridine-6-carboxylate (3 g, 6.87 mmol) in toluene (50 mL) was added trimethylaluminium (990.83 mg, 13.74 mmol) at 0° C. and the reaction was heated at 110° C. for 8 h. Upon completion of the reaction, the reaction mixture was cooled to room temperature and concentrated in vacuo. The residue was added to 10 V water and NaHCO₃ solution (2 V) and then filtered through celite, which was washed with ethyl acetate. The organic layer was separated and the aqueous layer was extracted with ethyl acetate (2×10 V), dried over anhydrous Na₂SO₄ and concentrated under reduced pressure. The crude product was purified by column chromatography (silica gel, 40% EA/Pet Ether) to give 2-[4-(benzyloxymethyl)cyclohexyl]-7-isopropoxy-N-[6-(trifluoromethyl)-2-pyridyl]imidazo[1,2-a]pyridine-6-carboxamide (2.0 g, 3.11 mmol, 45.20% yield). LCMS (ES⁺): m/z 567.85 [M+H]⁺

### Step-8:

To a solution of 2-[4-(benzyloxymethyl)cyclohexyl]-7-isopropoxy-N-[6-(trifluoromethyl)-2-pyridyl]imidazo[1,2-a]pyridine-6-carboxamide (4 g, 7.06 mmol), Palladium/C (4 g, 7.06 mmol) and HCl (1.29 g, 35.30 mmol, 1.61 mL) in methanol (50 mL) were added at room temperature and the reaction mixture was stirred under hydrogen atmosphere (balloon) for 2 h. Upon completion of the reaction, the reaction mixture was filtered through celite and concentrated under reduced pressure. The crude was basified with aq. NaHCO₃ solution (5 V) and extracted with ethyl acetate (2×10 V), dried over anhydrous Na₂SO₄ and concentrated in vacuo to afford 2-[4-(hydroxymethyl)cyclohexyl]-7-isopropoxy-N-[6-(trifluoromethyl)-2-pyridyl]imidazo[1,2-a]pyridine-6-carboxamide (3.2 g, 4.77 mmol, 67.54% yield). LCMS (ES⁺): m/z 477.42 [M+H]⁺

### Synthesis of 2-((1r,4r)-4-((benzyloxy)methyl)cyclohexyl)-6-isopropoxy-2H-indazole-5-carboxylic acid

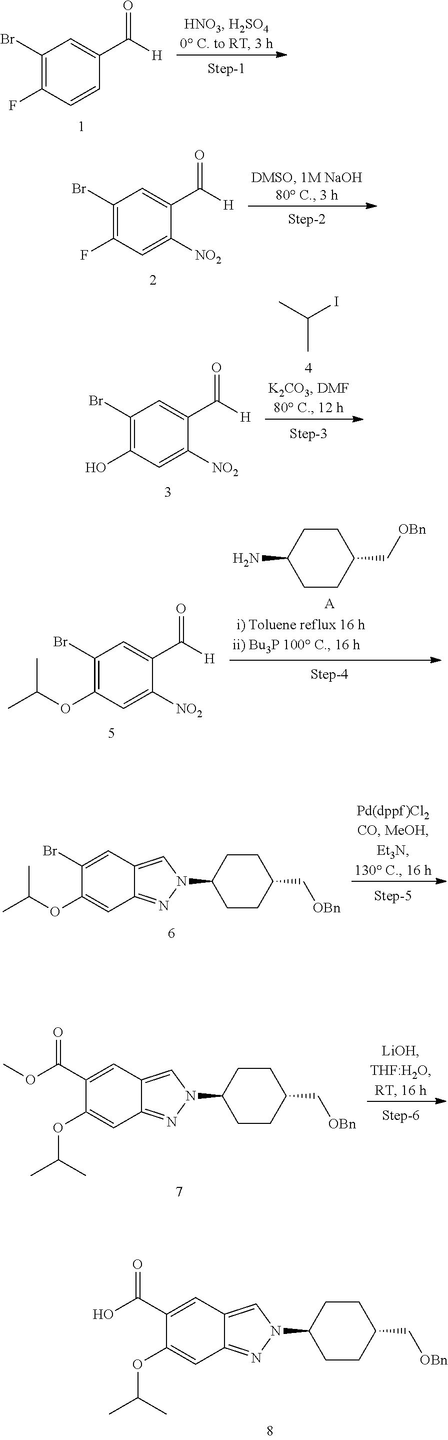

### Step-1:

3-Bromo-4-fluoro-benzaldehyde 1 (500 g, 2.46 mol) was charged in 5 Lit 3-neck RBF fitted with mechanical stirrer. Sulphuric acid (3 L) was added in one portion at RT and cooled to 0° C. Fuming nitric acid, 95% (500 mL) was added drop wise over the period of 1 h during which the reaction mixture slowly turned into a thick viscous brown liquid. The reaction was allowed to stir at RT over the period of 2 h. The reaction mixture was poured slowly into cold water (10 L) and stirred vigorously for 3 h. The precipitated solid was filtered-off and the crude compound was again suspended in water (10 L), stirred vigorously, filtered and dried under vacuum to afford crude product (580 g). The crude compound (580 g) was suspended in pet ether (4 L), stirred vigorously, and filtered-off. The same exercise was repeated 2 more times with pet ether (2×1 L) and filtered to obtain 5-bromo-4-fluoro-2-nitro-benzaldehyde 2 (372 g, 61% yield) as pale-yellow solid. ¹H NMR (400 MHz, DMSO-d₆) δ 10.38 (s, 1H), 8.22 (d, J=6.4 Hz, 1H), 7.91 (d, J=7.6 Hz, 1H).

### Step-2:

To a stirred solution of 5-bromo-4-fluoro-2-nitro-benzaldehyde (2) (250 g, 1.01 mol) in DMSO (2.5 L) was added aqueous 1N sodium hydroxide (2 L) at RT and stirred at 80° C. for 3 h. On completion, the reaction mixture was cooled to RT and quenched slowly with cold water (7 L). The aqueous layer was extracted with ethyl acetate (2×2 L) until the non-polar impurities was completely removed. The aqueous layer was acidified with aqueous 2 M HCl solution (5 L) (P^{H}˜2-3) and the product was extracted with MTBE (2×2 L). The combined organic layer was washed with water (500 mL), dried over anhydrous Na₂SO₄, filtered and concentrated under reduced pressure to afford 5-bromo-4-hydroxy-2-nitro-benzaldehyde 3 (212 g, 85% yield) as brown solid. ¹H NMR (400 MHz, DMSO-d₆): δ 12.39 (bs, 1H), 10.03 (s, 1H), 8.08 (s, 1H), 7.52 (s, 1H).

### Step-3:

To a stirred solution of 5-bromo-4-hydroxy-2-nitro-benzaldehyde 3 (210 g, 853.61 mmol) in DMF (1.5 L) was added potassium carbonate (236 g, 1.71 mol) at RT followed by the addition of isopropyl iodide 4 (170 mL, 1.71 mol). The reaction mixture was then stirred at 80° C. for 12 h. On completion, the reaction mixture was poured into ice cold water (5 L) and stirred vigorously for 1 h. The precipitated solid was filtered and dried to obtain (262 g) crude product. The crude product was suspended in diethyl ether (100 mL), cooled to 10° C. and the slurry was filtered immediately to afford 5-bromo-4-isopropoxy-2-nitro-benzaldehyde (102 g) as first crop. The filtrate was concentrated under reduced pressure and the crude compound (128 g) was again suspended in diethyl ether (50 mL), cooled to 10° C. and the slurry was filtered immediately to afford 5-bromo-4-isopropoxy-2-nitro-benzaldehyde (68 g) as second crop. Both crops were blended together to yield 5-bromo-4-isopropoxy-2-nitro-benzaldehyde 5 (170 g, 69% yield) as yellow solid. LCMS (ES⁺): m/z 286.05 [M+H]⁺. ¹H NMR (400 MHz, DMSO-d₆): δ 10.30 (s, 1H), 8.21 (s, 1H), 7.51 (s, 1H), 4.81-4.73 (m, 1H), 1.48 (d, J=6 Hz, 6H).

### Step-4:

To a stirred solution of 5-bromo-4-isopropoxy-2-nitro-benzaldehyde 5 (100 g, 347.11 mmol) in toluene (1 L) was added (1r, 4r) 4-(benzyloxymethyl)cyclohexanamine A (76 g, 347.11 mmol) and 4 Å molecular Sieves (100 g, 347.11 mmol) at RT. The reaction mixture was heated at 130° C. for 16 h. After the confirmation of Schiff base formation by TLC, the reaction mixture was cooled to 0° C. and tributyl phosphine (147 g, 728.93 mmol, 0.18 L) was added dropwise and heated the reaction at 130° C. (External bath temperature) for another 16 h. After the completion, the reaction mixture was filtered through celite bed and washed using ethyl acetate (500 mL). The filtrate was diluted with water (500 mL) and extracted with ethyl acetate (3×500 mL). The combined organic layer was dried over sodium sulphate, filtered and concentrated under reduced pressure. The crude compound was purified by silica gel (100-200 mesh, 0-10% ethyl acetate in pet ether as eluent gradient) to afford 2-((1r,4r)-2-[4-(benzyloxymethyl)cyclohexyl]-5-bromo-6-isopropoxy-indazole 6 (80 g, 173.85 mmol, 50.09% yield) as brown solid. LCMS (ES⁺): m/z 457.97 [M+H]⁺

### Step-5:

The stirred solution of 2-[4-(benzyloxymethyl)cyclohexyl]-5-bromo-6-isopropoxy-indazole 6 (80 g, 174.90 mmol) in methanol (600 mL) was added triethylamine (88 g, 874.50 mmol, 0.122 L) at RT in an autoclave. The reaction mixture was degassed with argon gas for 10 min. by purging and added Pd(dppf)Cl₂ (6.4 g, 8.75 mmol) and stirred the reaction mixture at 130° C. under carbon monoxide (300 PSI) atmosphere for 16 h. After completion, the reaction mixture was cooled, filtered through celite pad and washed with ethyl acetate (100 mL). The filtrate was concentrated under reduced pressure to get crude product. The resulting crude was purified by silica gel (100-200 mesh) by using 45%-60% ethyl acetate in pet ether as mobile phase to afford methyl 2-[4-(benzyloxymethyl)cyclohexyl]-6-isopropoxy-indazole-5-carboxylate 7 (65 g, 133.78 mmol, 76.49% yield) as pale brown viscous solid. LCMS (ES⁺): m/z 437.37 [M+H]⁺

### Step-6:

To a stirred solution of methyl 2-[4-(benzyloxymethyl)cyclohexyl]-6-isopropoxy-indazole-5-carboxylate 7 (50 g, 114.54 mmol) in methanol (377 mL) was added a solution of lithium hydroxide, monohydrate (19 g, 458.15 mmol, 12.73 mL) in water (377 mL) at 0° C. and stirred the reaction mixture at RT for 16 h. After completion, the reaction mixture was concentrated under high vacuum to get crude product. The resulting crude was acidified with 1N HCl up to ˜pH 2 and extracted with ethyl acetate (2×1 L). The combined organic layer was dried over sodium sulphate and concentrated under high vacuum to get crude product. The resulting crude was triturated with diethyl ether (200 mL), dried under high vacuum to afford 2-[4-(benzyloxymethyl)cyclohexyl]-6-isopropoxy-indazole-5-carboxylic acid 8 (38 g, 87.61 mmol, 76.49% yield) as off white solid. LCMS (ES⁺): m/z 423.48 [M+H]⁺

### Synthesis of 2-((1r,4r)-4-((benzyloxy)methyl)cyclohexyl)-N-(1-cyclopropyl-2-oxo-1,2-dihydropyridin-3-yl)-6-isopropoxy-2H-indazole-5-carboxamide

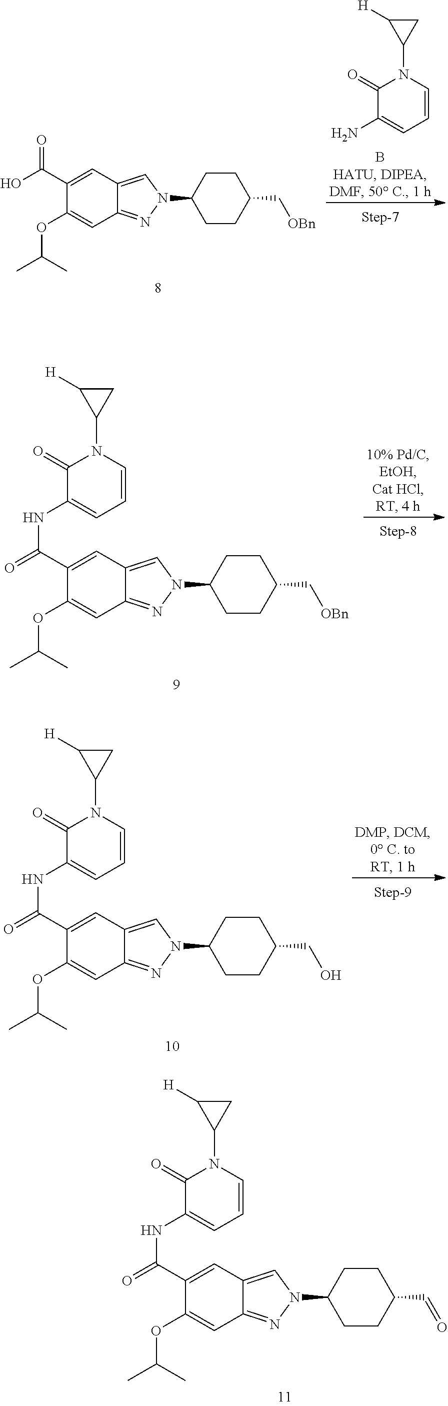

### Step-7:

To a stirred solution of 2-[4-(benzyloxymethyl)cyclohexyl]-6-isopropoxy-indazole-5-carboxylic acid 8 (11 g, 26.03 mmol) in DMF (110 mL) was added DIPEA (10.09 g, 78.09 mmol, 13.60 mL) followed by the addition of 3-amino-1-cyclopropyl-pyridin-2-one B (4.69 g, 31.24 mmol) and HATU (14.85 g, 39.05 mmol) at 0° C. and stirred the reaction mixture at 50° C. for 1 h. Upon completion, the reaction mixture was cooled to RT and quenched slowly with cold water (300 mL) and the aqueous layer was extracted with ethyl acetate (3×300 mL). The combined organic layer was separated, dried over sodium sulphate and concentrated under reduced pressure to get crude product (15 g). The resulting crude (15 g) was purified by silica gel (100-200 mesh, 80% ethyl acetate in pet ether as mobile phase) to afford 2-((1r,4r)-4-((benzyloxy)methyl)cyclohexyl)-N-(1-cyclopropyl-2-oxo-1,2-dihydropyridin-3-yl)-6-isopropoxy-2H-indazole-5-carboxamide 9 (9 g, 14.28 mmol, 62.32% yield) as yellow solid. LCMS (ES⁺): m/z 555.48 [M+H]⁺

### Step-8:

To a stirred solution of 2-[4-(benzyloxymethyl)cyclohexyl]-N-(1-cyclopropyl-2-oxo-3-pyridyl)-6-isopropoxy-indazole-5-carboxamide 9 (9 g, 16.23 mmol) in methanol (99.63 mL) and ethanol (99.63 mL) was added 10% Palladium on carbon 50% wet basis (9 g, 16.23 mmol) and hydrochloric acid, 36% w/w aqueous solution (591.60 mg, 16.23 mmol, 739.50 L) stirred the reaction mixture at 28° C. for 4 h under hydrogen atmosphere. Upon completion, the reaction mixture was filtered through celite pad and washed with 10% methanol in DCM (500 mL). The combined organic layer was washed with saturated solution of sodium bicarbonate (250 mL) dried over sodium sulphate and concentrated under reduced pressure to get crude product (10 g). The crude compound (10 g) was suspended in diethyl ether (500 mL), stirred vigorously for 1 h and the precipitated solid was filtered and dried to afford N-(1-cyclopropyl-2-oxo-3-pyridyl)-2-[4-(hydroxymethyl)cyclohexyl]-6-isopropoxy-indazole-5-carboxamide 10 (6.8 g, 12.59 mmol, 77.58% yield) as off white solid. LCMS (ES⁺): m/z 465.41 [M+H]⁺

### Step-9:

To a stirred solution of N-(1-cyclopropyl-2-oxo-3-pyridyl)-2-[4-(hydroxymethyl)cyclohexyl]-6-isopropoxy-indazole-5-carboxamide 10 (6.8 g, 14.64 mmol) in DCM (70 mL) was added Dess-Martin periodinane (15.52 g, 36.59 mmol) at 0° C. and stirred the reaction mixture at 28° C. for 1 h. Upon completion, the reaction mixture was filtered through celite pad and washed with DCM (500 mL). The combined organic layer was washed with saturated aqueous sodium bicarbonate (250 mL), dried over sodium sulphate and concentrated under reduced pressure to get crude product (8 g). The resulting crude product was purified by silica gel (100-200 mesh, 40% ethyl acetate in pet ether as mobile phase), the desired fraction was concentrated under reduced pressure to afford N-(1-cyclopropyl-2-oxo-3-pyridyl)-2-(4-formylcyclohexyl)-6-isopropoxy-indazole-5-carboxamide 11 (5 g, 6.16 mmol, 73% yield) as yellow oil. LCMS (ES⁺): m/z 463.36 [M+H]⁺

### Synthesis of N-(1-((1S,2R)-2-fluorocyclopropyl)-2-oxo-1,2-dihydropyridin-3-yl)-2-((1r,4S)-4-formylcyclohexyl)-6-isopropoxy-2H-indazole-5-carboxamide

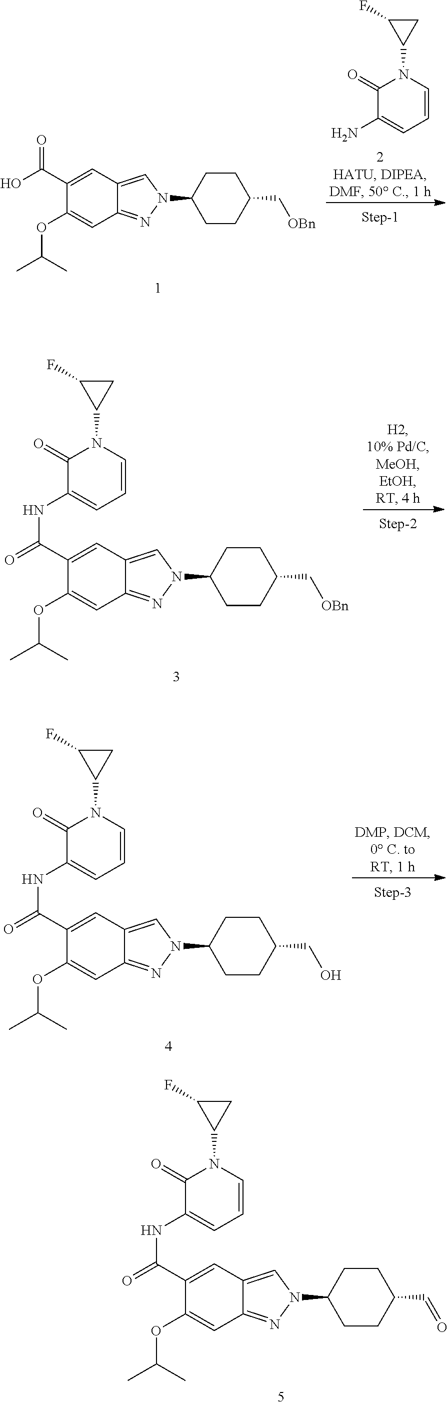

### Step-1:

To a stirred solution of 2-[4-(benzyloxymethyl)cyclohexyl]-6-isopropoxy-indazole-5-carboxylic acid 1 (1 g, 2.37 mmol)) in DMF (10 mL) was added DIPEA (305.89 mg, 2.37 mmol, 412.25 μL) followed by the addition of (1R)-3-amino-1-[(2S)-2-fluorocyclopropyl]pyridin-2-one 2 (801.46 mg, 2.84 mmol, TFA salt) and HATU (899.92 mg, 2.37 mmol) at 0° C. and stirred the reaction mixture at 50° C. for 1 h. Upon completion, the reaction mixture was cooled to RT and quenched slowly with cold water (150 mL) and the aqueous layer was extracted with ethyl acetate (3×100 mL). The combined organic layer was separated, dried over sodium sulphate and concentrated under reduced pressure to get crude product (1.5 g). The resulting crude (1.5 g) was purified by silica gel (100-200 mesh, 25% ethyl acetate in pet ether as mobile phase), the desired fractions were concentrated under reduced pressure to afford 2-[4-(benzyloxymethyl)cyclohexyl]-6-isopropoxy-N-[(1R)-2-oxo-1-[(2S)-2-fluorocyclopropyl]-3-pyridyl]indazole-5-carboxamide 3 (0.8 g, 377.18 μmol, 15.94% yield) as yellow solid. LCMS (ES⁺): m/z 573.55 [M+H]⁺

### Step-2:

To a stirred solution of 2-[4-(benzyloxymethyl)cyclohexyl]-6-isopropoxy-N-[(1R)-2-oxo-1-[(2S)-2-fluorocyclopropyl]-3-pyridyl]indazole-5-carboxamide 3 (0.8 g, 1.40 mmol) in methanol (4 mL) and ethanol (4 mL) was added 10% Palladium on carbon 50% wet basis (0.8 g). The reaction mixture was stirred at 28° C. for 4 h under hydrogen atmosphere. Upon completion, the reaction mixture was filtered through celite pad and washed with ethyl acetate (100 mL). The combined organic layer was concentrated under reduced pressure to get crude product (0.7 g). The crude compound (0.7 g) was suspended in diethyl ether (10 mL), stirred vigorously for 10 min and the precipitated solid was filtered and dried to afford 2-[4-(hydroxymethyl)cyclohexyl]-6-isopropoxy-N-[(1R)-2-oxo-1-[(2S)-2-fluorocyclopropyl]-3-pyridyl]indazole-5-carboxamide 4 (0.4 g, 770.91 μmol, 55.18% yield) as off white solid. LCMS (ES⁺): m/z 483.41 [M+H]⁺

### Step-3:

To a stirred solution of 2-[4-(hydroxymethyl)cyclohexyl]-6-isopropoxy-N-[(1R)-2-oxo-1-[(2S)-2-fluorocyclopropyl]-3-pyridyl]indazole-5-carboxamide 4 (3.5 g, 7.25 mmol) in DCM (35 mL) was added Dess-Martin periodinane (7.69 g, 18.13 mmol) at 0° C. and stirred the reaction mixture at 28° C. for 1 h. Upon completion, the reaction mixture was filtered through celite pad and washed with DCM (500 mL). The combined organic layer was washed with saturated aqueous sodium bicarbonate (300 mL), dried over sodium sulfate and concentrated under reduced pressure to get crude product (4 g). The resulting crude product was purified by silica gel (100-200 mesh, 60% ethyl acetate in pet ether as mobile phase), the desired fraction was concentrated under reduced pressure to afford N-(1-((1S,2R)-2-fluorocyclopropyl)-2-oxo-1,2-dihydropyridin-3-yl)-2-((1r,4S)-4-formylcyclohexyl)-6-isopropoxy-2H-indazole-5-carboxamide 5 (2.3 g, 4.79 mmol, 65.99% yield) as yellow solid. LCMS (ES⁺): m/z 481.57 [M+H]⁺

### Synthesis of 2-((1r,4r)-4-formylcyclohexyl)-6-isopropoxy-N-(pyrazolo[1,5-a]pyrimidin-3-yl)-2H-indazole-5-carboxamide

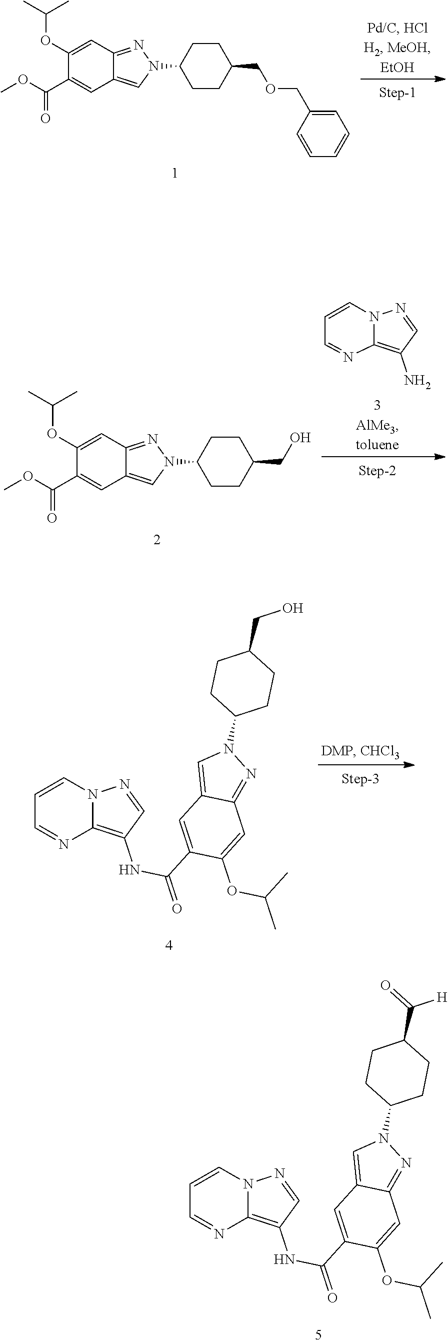

### Step-1:

To a stirred solution of methyl 2-[4-(benzyloxymethyl)cyclohexyl]-6-isopropoxy-indazole-5-carboxylate 1 (500 mg, 1.15 mmol) in methanol (10 mL) and ethanol (10 mL) was added palladium on activated carbon, 10 wt. %, (500 mg, 1.15 mmol) and hydrogen chloride (41.76 mg, 1.15 mmol, 0.6 mL) at room temperature. The reaction mixture was stirred under hydrogen atmosphere (balloon pressure) for 4 h. Subsequently, it was filtered through celite bed and washed with methanol (200 mL). The filtrate was concentrated under reduced pressure to give a colorless gel, which was dissolved in ethyl acetate (200 mL). The organic layer was washed with NaHCO₃ solution (100 mL), and concentrated under reduced pressure to yield the crude product, which was purified by column chromatography using silica gel (100-200 mesh) and 0-60% EtOAc in Pet. ether as eluent to afford methyl 2-[4-(hydroxymethyl)cyclohexyl]-6-isopropoxy-indazole-5-carboxylate 2 (330 mg, 781.13 μmol, 68.20% yield) as white solid. LCMS (ES⁺): m/z 347.54(M+H)⁺

### Step-2:

To a stirred solution of methyl 2-[4-(hydroxymethyl)cyclohexyl]-6-isopropoxy-indazole-5-carboxylate 2 (320 mg, 923.73 μmol) in toluene (5 mL) was added pyrazolo[1,5-a]pyrimidin-3-amine 3 (185.86 mg, 1.39 mmol) and trimethylaluminum solution 2.0 M in toluene (66.59 mg, 923.73 μmol, 1 mL) at 0° C. The reaction mixture was stirred at 100° C. for 16 hr. The reaction mixture was quenched with NH₄Cl (100 mL) and extracted with ethyl acetate (3×150 mL). The combined organic layer was dried over anhydrous Na₂SO₄ and concentrated. The crude compound was purified by (silica gel mesh 100-200, and product eluted with 0-60% ethyl acetate in pet ether-ethyl acetate) column chromatography to afford 2-[4-(hydroxymethyl)cyclohexyl]-6-isopropoxy-N-pyrazolo[1,5-a]pyrimidin-3-yl-indazole-5-carboxamide 4 (280 mg, 555.61 μmol, 60.15% yield). LCMS (ES⁺): m/z 449.66 [M+H]⁺

### Step-3:

To a solution of 2-[4-(hydroxymethyl)cyclohexyl]-6-isopropoxy-N-pyrazolo[1,5-a]pyrimidin-3-yl-indazole-5-carboxamide (270 mg, 601.98 μmol)and chloroform (10 mL) was added Dess-Martin periodinane (510.65 mg, 1.20 mmol). The reaction mixture was stirred at 25° C. for 2 hr. Subsequently, the reaction mixture was quenched with NaHCO₃ (50 mL) and extracted with ethyl acetate (2×150 mL), the organic layer was concentrated in vacuo to afford 2-(4-formylcyclohexyl)-6-isopropoxy-N-pyrazolo[1,5-a]pyrimidin-3-yl-indazole-5-carboxamide 5 (250 mg, 526.32 μmol, 87.43% yield) as gummy oil. LCMS (ES⁺): m/z 447.64 [M+H]⁺

### 2-(4-formylcyclohexyl)-6-isopropoxy-N-(6-methylpyrazolo[1,5-a]pyrimidin-3-yl) indazole-5-carboxamide

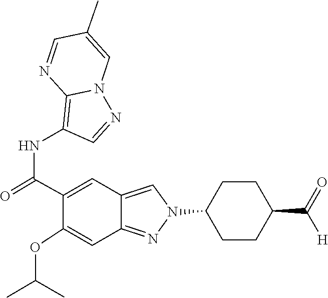 [SMILES: Cc1cnc2c(NC(=O)c3cc4cn([C@H]5CC[C@H](C=O)CC5)nc4cc3OC(C)C)cnn2c1]

Synthesis was identical to that of 2-((1r,4r)-4-formylcyclohexyl)-6-isopropoxy-N-(pyrazolo[1,5-a]pyrimidin-3-yl)-2H-indazole-5-carboxamide, except 6-methylpyrazolo[1,5-a]pyrimidin-3-amine was used in Step-2. LCMS (ES⁺): m/z 461.59 [M+H]⁺

### Synthesis of N-[1-[(1S,2R)-2-fluorocyclopropyl]-2-oxo-3-pyridyl]-6-isopropoxy-2-(4-piperidyl) indazole-5-carboxamide

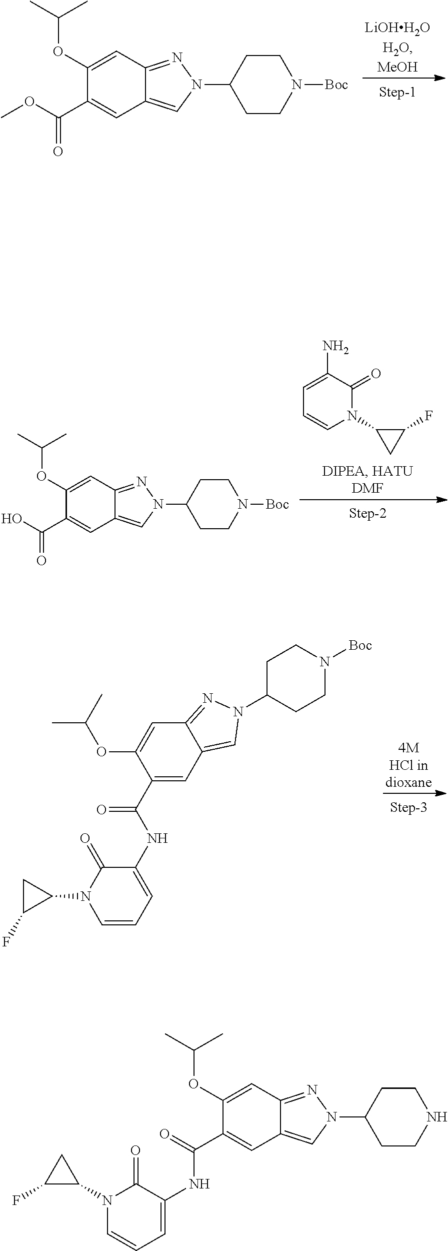

### Step-1:

To a stirred solution of methyl 2-(1-tert-butoxycarbonyl-4-piperidyl)-6-isopropoxy-indazole-5-carboxylate (20 g, 47.90 mmol) in water (40 mL) was added a solution of lithium hydroxide monohydrate, 98% (8.04 g, 191.62 mmol) in methanol (160 mL) at 0° C. and the reaction mixture was stirred at 25° C. for 16 h. After completion of the reaction, the reaction mixture was concentrated under high vacuum and the residue was acidified with 1.5N HCl and extracted with ethyl acetate. The combined organic layer was separated, dried over sodium sulfate and concentrated under high vacuum to give the crude product, which was triturated with diethyl ether to afford 2-(1-tert-butoxycarbonyl-4-piperidyl)-6-isopropoxy-indazole-5-carboxylic acid (19 g, 46.53 mmol, 97.12% yield) as an off-white solid. LCMS (ES⁺): m/z 404.38 [M+H]⁺

### Step-2:

To a stirred solution of 2-(1-tert-butoxycarbonyl-4-piperidyl)-6-isopropoxy-indazole-5-carboxylic acid (5 g, 12.39 mmol) in DMF (50 mL) was added DIPEA (4.80 g, 37.18 mmol, 6.48 mL) followed by 3-amino-1-[(1S,2R)-2-fluorocyclopropyl]pyridin-2-one (2.54 g, 12.39 mmol, HCl salt) and HATU (7.07 g, 18.59 mmol) at 0° C. and the reaction mixture was stirred at 50° C. for 16 h. After completion of the reaction, the reaction mixture was diluted with water (250 mL) and extracted with ethyl acetate (150×3 mL). The combined organic layer was separated, dried over sodium sulfate and concentrated under reduced pressure to give the crude product, which was purified by column chromatography (100-200 mesh silica gel, 80% ethyl acetate in pet ether as mobile phase) to afford tert-butyl 4-[6-isopropoxy-5-[[2-oxo-1-[(1S,2R)-2-fluorocyclopropyl]-3-pyridyl]carbamoyl]indazol-2-yl]piperidine-1-carboxylate (3.8 g, 6.45 mmol, 52.06% yield) as a yellow solid. LCMS (ES⁺): m/z 554.78 [M+H]⁺

### Step-3:

To the stirred solution of tert-butyl 4-[5-[[1-[(1S,2R)-2-fluorocyclopropyl]-2-oxo-3-pyridyl]carbamoyl]-6-isopropoxy-indazol-2-yl]piperidine-1-carboxylate (0.2 g, 361.26 μmol) in DCM (5 mL) was added 4.0M hydrogen chloride in 1,4-dioxane (2 mL) at 0° C. and the reaction was stirred for 2 h at 25° C. After consumption of the starting material, the solvent was removed under reduced pressure to obtain the crude compound, which was triturated in diethyl ether (5 mL). The diethyl ether layer was decanted and the product was dried under reduced pressure to afford N-[1-[(1S,2R)-2-fluorocyclopropyl]-2-oxo-3-pyridyl]-6-isopropoxy-2-(4-piperidyl) indazole-5-carboxamide (0.18 g, 351.10 μmol, 97.19% yield, HCl salt) as a brown solid. LCMS (ES⁺): m/z 454.47 [M+H]⁺

### 6-isopropoxy-2-(4-piperidyl)-N-pyrazolo[1,5-a]pyrimidin-3-yl-indazole-5-carboxamide

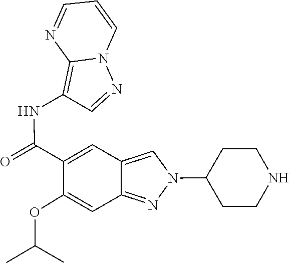 [SMILES: CC(C)Oc1cc2nn(C3CCNCC3)cc2cc1C(=O)Nc1cnn2cccnc12]

Synthesis was identical to that of N-[1-[(1S,2R)-2-fluorocyclopropyl]-2-oxo-3-pyridyl]-6-isopropoxy-2-(4-piperidyl) indazole-5-carboxamide, except using pyrazolo[1,5-a]pyrimidin-3-amine in Step-2. LCMS (ES⁺): m/z 421.39 [M+H]⁺

### Tert-butyl 4-[7-isopropoxy-6-(pyrazolo[1,5-a]pyrimidin-3-ylcarbamoyl)imidazo[1,2-a]pyridin-2-yl]piperidine-1-carboxylate

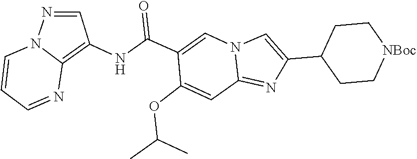 [SMILES: CC(C)Oc1cc2nc(C3CCN(C(=O)OC(C)(C)C)CC3)cn2cc1C(=O)Nc1cnn2cccnc12]

Synthesis was identical to that of tert-butyl 4-(7-isopropoxy-6-(pyrazolo[1,5-a]pyrimidin-3-ylcarbamoyl)imidazo[1,2-a]pyridin-2-yl)piperidine-1-carboxylate, except using pyrazolo[1,5-a]pyrimidin-3-amine in Step-2. LC-MS (ES⁺): m/z 520.51 [M+H]⁺.

### Synthesis of tert-butyl 4-[7-isopropoxy-6-[[2-oxo-1-[(1S,2R)-2-fluorocyclopropyl]-3-pyridyl]carbamoyl]imidazo[1,2-a]pyridin-2-yl]piperidine-1-carboxylate

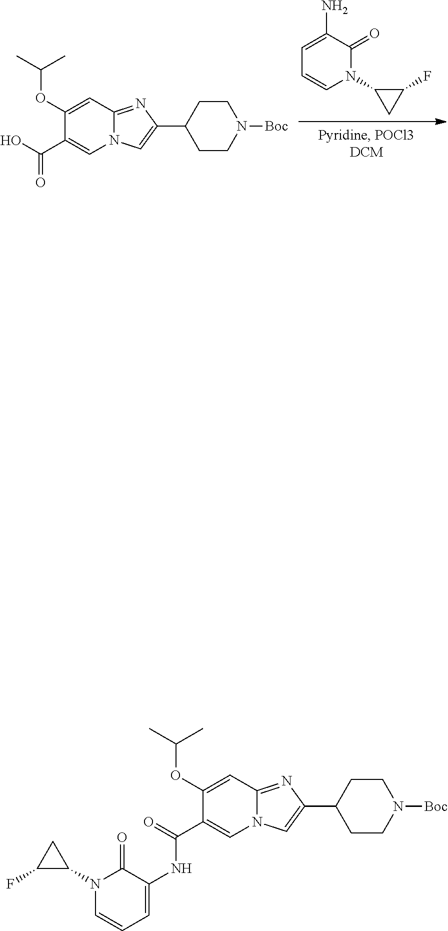 [SMILES: CC(C)Oc1cc2nc(C3CCN(C(=O)OC(C)(C)C)CC3)cn2cc1C(=O)O ; Nc1cccn([C@H]2C[C@H]2F)c1=O ; CC(C)Oc1cc2nc(C3CCN(C(=O)OC(C)(C)C)CC3)cn2cc1C(=O)Nc1cccn([C@H]2C[C@H]2F)c1=O]

A stirred solution of 3-amino-1-[(1S,2R)-2-fluorocyclopropyl]pyridin-2-one (2 g, 9.77 mmol, HCl salt), 2-(1-tert-butoxycarbonyl-4-piperidyl)-7-isopropoxy-imidazo[1,2-a]pyridine-6-carboxylic acid (4.34 g, 10.75 mmol) in DCM (60 mL) was cooled to 0° C. before pyridine (19.33 g, 244.34 mmol, 19.76 mL) and phosphoryl trichloride (4.50 g, 29.32 mmol, 2.74 mL) were added at this temperature. The reaction mixture was stirred for 2 hours at the same temperature. Upon completion of the reaction, the reaction mixture was diluted with water and extracted with DCM. The organic layer was dried over anhydrous sodium sulfate, filtered and concentrated under reduced pressure to give the crude product, which was purified by flash column chromatography (silica gel, 2-3% MeOH in DCM) to afford tert-butyl 4-[7-isopropoxy-6-[[2-oxo-1-[rac-(1S,2R)-2-fluorocyclopropyl]-3-pyridyl]carbamoyl]imidazo[1,2-a]pyridin-2-yl]piperidine-1-carboxylate (3.6 g, 4.63 mmol, 47.36% yield) as a brown solid. LC-MS (ES⁺): m/z 554.48 [M+H]⁺

### N-(1-cyclopropyl-2-oxo-3-pyridyl)-2-[4-(hydroxymethyl)cyclohexyl]-7-isopropoxy-imidazo[1,2-a]pyridine-6-carboxamide

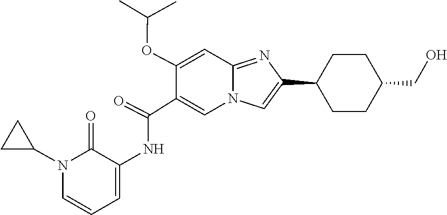 [SMILES: CC(C)Oc1cc2nc([C@H]3CC[C@H](CO)CC3)cn2cc1C(=O)Nc1cccn(C2CC2)c1=O]

Synthesis was identical to that of 6-(difluoromethyl)-N-[2-[4-(hydroxymethyl)cyclohexyl]-7-isopropoxy-imidazo[1,2-a]pyridin-6-yl]pyridine-2-carboxamide, except using 3-amino-1-cyclopropyl-pyridin-2-one in Step-4. LCMS (ES⁺): m/z 465.43 [M+H]⁺

### 2-[4-(hydroxymethyl)cyclohexyl]-7-isopropoxy-N-[2-oxo-1-[(1R,2S)-2-fluorocyclopropyl]-3-pyridyl]imidazo[1,2-a]pyridine-6-carboxamide

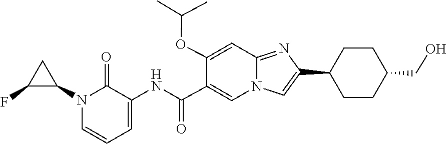 [SMILES: CC(C)Oc1cc2nc([C@H]3CC[C@H](CO)CC3)cn2cc1C(=O)Nc1cccn([C@@H]2C[C@@H]2F)c1=O]

Synthesis was identical to that of 6-(difluoromethyl)-N-[2-[4-(hydroxymethyl)cyclohexyl]-7-isopropoxy-imidazo[1,2-a]pyridin-6-yl]pyridine-2-carboxamide, except using 3-amino-1-[(1S,2R)-2-fluorocyclopropyl]pyridin-2-one in Step-4. LCMS (ES⁺): m/z 483.68 [M+H]⁺

### 1-[5-fluoro-1-methyl-6-[(3S,4R)-3-hydroxy-4-piperidyl]indazol-3-yl]hexahydropyrimidine-2,4-dione

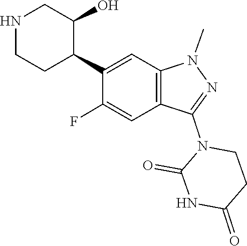 [SMILES: Cn1nc(N2CCC(=O)NC2=O)c2cc(F)c([C@H]3CCNC[C@H]3O)cc21]

This compound was prepared substantially following the synthesis of 1-(6-((3R,4S)-3-hydroxypiperidin-4-yl)-1-methyl-1H-indazol-3-yl)dihydropyrimidine-2,4(1H,3H)-dione, except using 4-bromo-2,5-difluoro-benzonitrile as starting material. LCMS (ES⁺): m/z 362.30 [M+H]⁺¹H NMR (400 MHz, DMSO-d₆): δ 10.56 (s, 1H), 7.46 (d, J=5.6 Hz, 1H), 7.41 (d, J=11.2 Hz, 1H), 5.49 (d, J=4.4 Hz, 1H), 4.08 (s, 1H), 4.00 (s, 3H) 3.90 (t, 2H), 3.39 (s, 1H), 3.27-3.11 (m, 4H) 2.77 (m, 2H), 2.42 (m, 1H), 1.76 (d, J=11.2 Hz, 1H).

### 2-[4-(hydroxymethyl)cyclohexyl]-7-isopropoxy-N-[2-oxo-1-[(1S,2R)-2-fluorocyclopropyl]-3-pyridyl]imidazo[1,2-a]pyridine-6-carboxamide

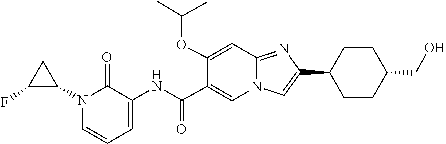 [SMILES: CC(C)Oc1cc2nc([C@H]3CC[C@H](CO)CC3)cn2cc1C(=O)Nc1cccn([C@H]2C[C@H]2F)c1=O]

This compound was prepared substantially following the synthesis of 6-(difluoromethyl)-N-[2-[4-(hydroxymethyl)cyclohexyl]-7-isopropoxy-imidazo[1,2-a]pyridin-6-yl]pyridine-2-carboxamide, using 3-amino-1-[(1S,2R)-2-fluorocyclopropyl]pyridin-2-one instead of 6-(difluoromethyl)pyridin-2-amine in Step-4. LCMS (ES⁺): m/z 483.48 [M+H]⁺

### 2-[1-[5-(2,6-dioxo-3-piperidyl)-3-fluoro-2-pyridyl]-4-piperidyl]acetic acid

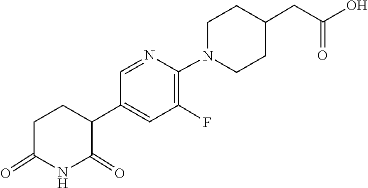 [SMILES: O=C(O)CC1CCN(c2ncc(C3CCC(=O)NC3=O)cc2F)CC1]

This compound was prepared substantially following the synthesis of 2-[1-[5-(2,6-dioxo-3-piperidyl)-2-pyridyl]-4-piperidyl]acetic acid, except using 5-bromo-2,3-difluoro-pyridine as starting material. LCMS (ES⁺): m/z 350.22 [M+H]⁺

### Synthesis of 7-isopropoxy-N-[2-oxo-1-[(1S,2R)-2-fluorocyclopropyl]-3-pyridyl]-2-(4-piperidyl)imidazo[1,2-a]pyrimidine-6-carboxamide

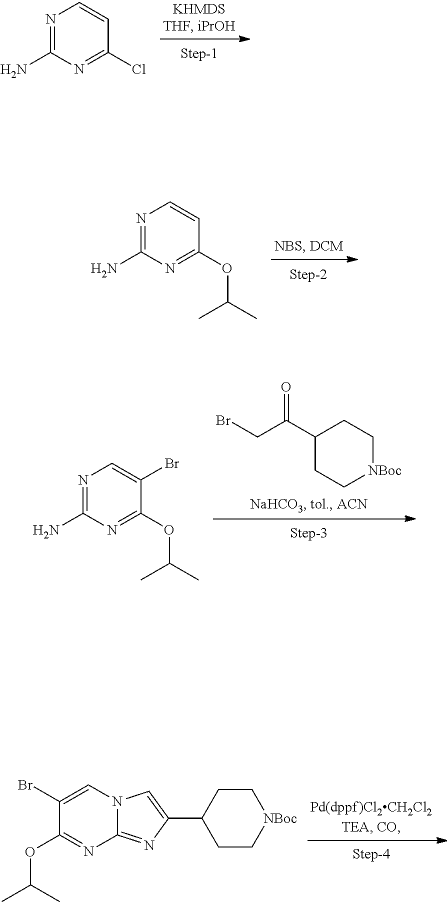

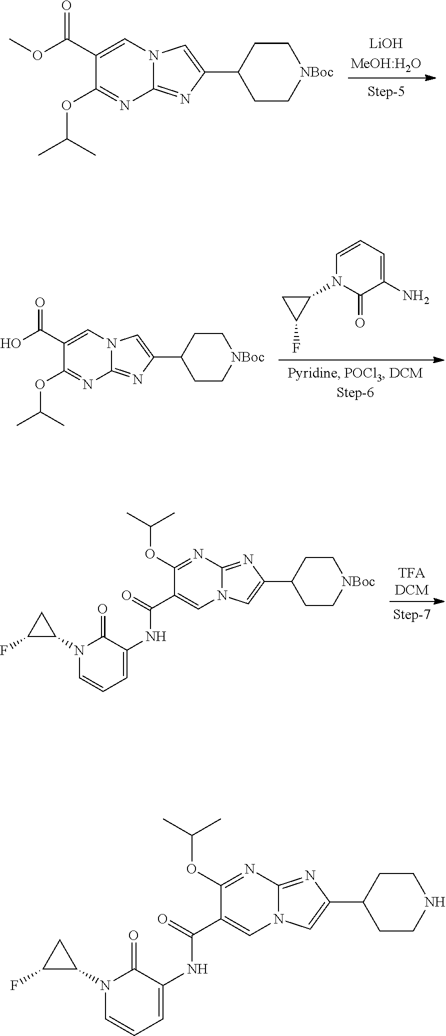

### Step-1:

To a stirred solution of 4-chloropyrimidin-2-amine (70 g, 540.34 mmol) in THF was added KHMDS in THF (1 M, 2.16 L) at 0° C. followed by the addition of propan-2-ol (162.36 g, 2.70 mol, 206.83 mL). The reaction mixture was allowed to stir at room temperature for 8 h. After complete consumption of the starting material, the reaction mixture was quenched with ammonium chloride solution (1 L) and extracted with ethyl acetate (2×1 L). The combined organic layers were dried over anhydrous Na₂SO₄ and concentrated under reduced pressure to give 4-isopropoxypyrimidin-2-amine (70 g, 411.28 mmol, 76.11% yield) as a brown solid.

### Step-2:

To a stirred solution of 4-isopropoxypyridin-2-amine (70 g, 457 mol) in DCM (700 mL) was added NBS (65 g, 365 mol) portion wise while maintaining the temp <5° C. The reaction mixture was allowed to stir at room temperature for 3 h. After complete consumption of the starting material, the reaction mixture was quenched with cold water (500 mL) and extracted with ethyl acetate (2×500 mL). The combined organic layers were washed with brine (500 mL), dried over anhydrous sodium sulfate, filtered and concentrated under reduced pressure. The crude product was triturated with DCM and pet ether (1:5) to afford 5-bromo-4-isopropoxy-pyrimidin-2-amine (70 g, 274.48 mmol, 60.06% yield) as a brown solid. LCMS (ES⁺): m/z 233.69 [M+H]⁺

### Step-3:

A stirred solution of 5-bromo-4-isopropoxy-pyrimidin-2-amine (15 g, 64.63 mmol) and tert-butyl 4-(2-bromoacetyl) piperidine-1-carboxylate (39.58 g, 129.27 mmol) in ethanol (140 mL) was purged with Nitrogen gas for 10 min. The resulting reaction mixture was stirred in a steel bomb at 60° C. for 16 h. After complete consumption of the starting material, the reaction mixture was concentrated under reduced pressure to give the crude product, which was triturated with diethyl ether (50 mL) to afford methyl tert-butyl 4-(6-bromo-7-isopropoxy-imidazo[1,2-a]pyrimidin-2-yl)piperidine-1-carboxylate (16 g, 35.69 mmol, 55% yield) as an off-white solid. LCMS (ES⁺): m/z 439.22 [M+H]⁺

### Step-4:

A stirred solution of tert-butyl 4-(6-bromo-7-isopropoxy-imidazo[1,2-a] pyrimidin-2-yl)piperidine-1-carboxylate (16 g, 36.42 mmol) in MeOH (160 mL) in an autoclave was purged with N₂ gas for 10 min followed by the addition of Pd(dppf)Cl₂ (2.66 g, 3.64 mmol), sodium acetate, anhydrous (5.97 g, 72.84 mmol). The reaction mixture was stirred at 100° C. under CO gas (300 psi) for 36 h. After complete consumption of the starting material, the reaction mixture was filtered through a pad of celite and washed with ethyl acetate (100 mL). The filtrate was concentrated under reduced pressure to give the crude product, which was purified by column chromatography using silica gel (100-200 mesh) and 30 to 60% EtOAc in Pet ether as eluent to afford methyl 2-(1-tert-butoxycarbonyl-4-piperidyl)-7-isopropoxy-imidazo[1,2-a] pyrimidine-6-carboxylate (8 g, 17.59 mmol, 48.29% yield) as an off-white solid. LCMS (ES⁺): m/z 419.64 [M+H]⁺

### Step-5:

A stirred solution of methyl 2-(1-tert-butoxycarbonyl-4-piperidyl)-7-isopropoxy-imidazo[1,2-a] pyrimidine-6-carboxylate (5 g, 11.95 mmol) in a mixture of methanol (25 mL), THE (40 mL) and water (25 mL) was cooled to 0° C. followed by the addition of lithium hydroxide monohydrate (2.51 g, 59.74 mmol). The resulting mixture was allowed to stir at room temperature for 3 h. After complete consumption of the starting material, the reaction mixture was concentrated under reduced pressure to get residue which was diluted with water (20 mL) and acidified with citric acid solution to give a precipitate, which was filtered, washed with water and dried to afford 2-(1-tert-butoxycarbonyl-4-piperidyl)-7-isopropoxy-imidazo[1,2-a]pyrimidine-6-carboxylic acid (4 g, 8.60 mmol, 72.01% yield) as an off-white solid. LCMS (ES⁺): m/z 405.33 [M+H]⁺

### Step-6:

To a stirred solution of 2-(1-tert-butoxycarbonyl-4-piperidyl)-7-isopropoxy-imidazo[1,2-a]pyrimidine-6-carboxylic acid (2 g, 4.94 mmol),3-amino-1-[(1S,2R)-2-fluorocyclopropyl]pyridin-2-one (1.62 g, 7.91 mmol, HCl salt) in DCM (19.43 mL) was added pyridine (9.78 g, 123.62 mmol, 10.00 mL) and stirred for 5 minutes. Then phosphoryl chloride (2.27 g, 14.83 mmol, 1.39 mL) was added at 0° C. and stirred for 1 h at the same temperature. Upon completion of the reaction, the reaction mixture was diluted with water (20 ML) and extracted with DCM (30 mL×2). The organic layer was dried over anhydrous sodium sulfate, filtered and concentrated under reduced pressure to afford tert-butyl 4-[7-isopropoxy-6-[[2-oxo-1-[(1S,2R)-2-fluorocyclopropyl]-3-pyridyl]carbamoyl]imidazo[1,2-a]pyrimidin-2-yl]piperidine-1-carboxylate (2 g, 3.43 mmol, 69.28% yield) as an off white solid. LCMS (ES⁺): m/z 555.58 [M+H]⁺

### Step-7:

To a stirred solution of tert-butyl 4-[7-isopropoxy-6-[[2-oxo-1-[(1S,2R)-2-fluorocyclopropyl]-3-pyridyl]carbamoyl]imidazo[1,2-a]pyrimidin-2-yl]piperidine-1-carboxylate (2 g, 3.61 mmol) in DCM (19.94 mL) was added trifluoroacetic acid (411.17 mg, 3.61 mmol, 277.82 μL) slowly at 0° C. and the reaction mixture was stirred at room temperature for 2 h. Upon completion of the reaction, the reaction mixture was concentrated under reduced pressure to afford 7-isopropoxy-N-[2-oxo-1-[(1S,2R)-2-fluorocyclopropyl]-3-pyridyl]-2-(4-piperidyl)imidazo[1,2-a]pyrimidine-6-carboxamide (1.9 g, 3.24 mmol, 89.90% yield, TFA salt). LCMS (ES⁺): m/z 455.34 [M+H]⁺

### Example 1

### Synthesis of 2-[4-[[4-[4-[(2,6-dioxo-3-piperidyl)amino]-2-fluoro-phenyl]-1-piperidyl]methyl]cyclohexyl]-7-isopropoxy-N-[6-(trifluoromethyl)-2-pyridyl]imidazo[1,2-a]pyridine-6-carboxamide

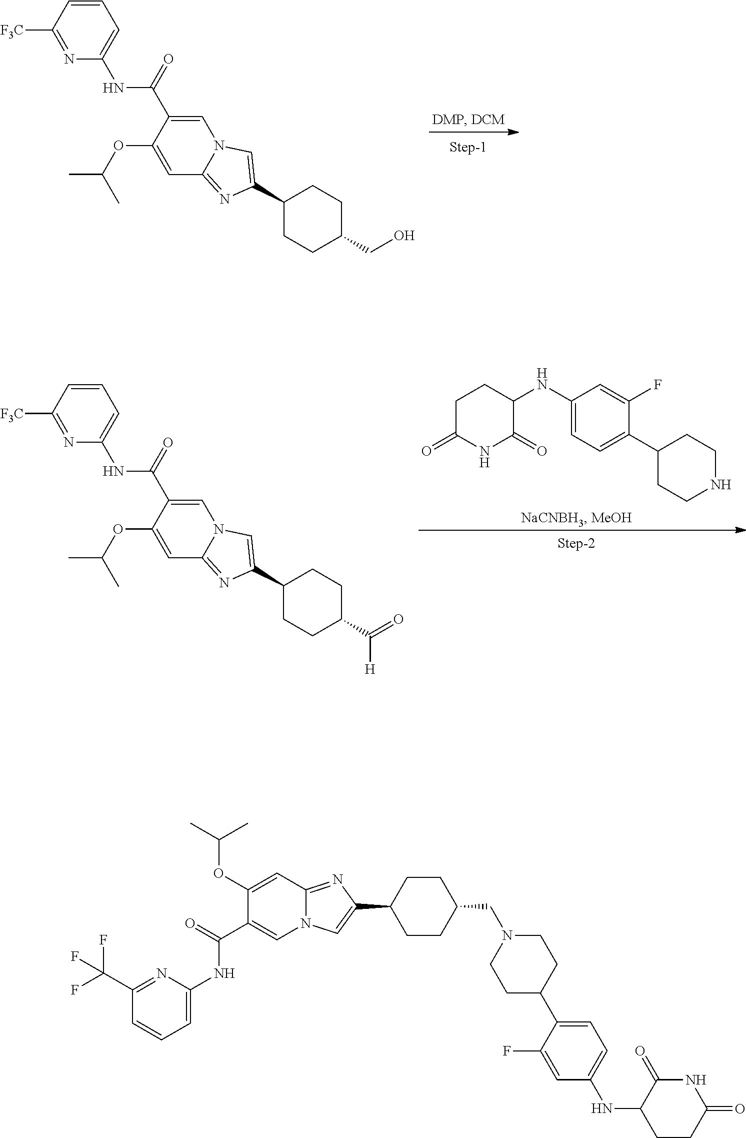

### Step-1:

To a stirred solution of 2-[4-(hydroxymethyl)cyclohexyl]-7-isopropoxy-N-[6-(trifluoromethyl)-2-pyridyl]imidazo[1,2-a]pyridine-6-carboxamide (1.50 g, 3.15 mmol) in DCM (50 mL) was added Dess-Martin Periodinane (4.01 g, 9.44 mmol) at 0-5° C. The reaction mixture was warmed up to 25° C. and stirred for an additional 3 hours. Progress of the reaction was monitored by LCMS/TLC. After completion, the reaction was quenched with saturated cold sodium bicarbonate solution, extracted with DCM. The organic layers were washed with brine, dried over anhydrous Na₂SO₄ and concentrated in vacuo to give the crude compound, which was purified by column chromatography (silica gel 100-200 mesh, 0-50% ethyl acetate in pet ether) to afford 2-(4-formylcyclohexyl)-7-isopropoxy-N-[6-(trifluoromethyl)-2-pyridyl]imidazo[1,2-a]pyridine-6-carboxamide (0.6 g, 1.10 mmol, 34.95% yield). LC-MS (ES⁺): m/z 475.41 [M+H]⁺.

### Step-2:

To a stirred solution of 2-(4-formylcyclohexyl)-7-isopropoxy-N-[6-(trifluoromethyl)-2-pyridyl]imidazo[1,2-a]pyridine-6-carboxamide (0.07 g, 147.53 μmol) and 3-[3-fluoro-4-(4-piperidyl)anilino]piperidine-2,6-dione TFA salt (74.24 mg, 177.04 μmol) in methanol (3 mL), triethylamine (14.93 mg, 147.53 μmol, 20.56 μL) was added. After stirring for 15 minutes, catalytic acetic acid (2.21 mg, 36.88 μmol) was added and the reaction mixture was heated at 60° C. for 4 hours. Then, the reaction mixture was cooled to 0° C. and sodium cyanoborohydride (18.54 mg, 295.06 μmol) was added. The reaction was maintained at room temperature until complete consumption of the starting material as evidenced by TLC. The reaction mixture was evaporated to dryness and purified using prep-HPLC. The collected fractions were then concentrated under reduced pressure and lyophilized to afford 2-[4-[[4-[4-[(2,6-dioxo-3-piperidyl)amino]-2-fluoro-phenyl]-1-piperidyl]methyl]cyclohexyl]-7-isopropoxy-N-[6-(trifluoromethyl)-2-pyridyl]imidazo[1,2-a]pyridine-6-carboxamide formic acid salt (22 mg, 25.23 μmol, 17.10% yield). ¹H NMR (401 MHz, DMSO-d₆) δ 10.98 (s, 1H), 10.78 (s, 1H), 9.10 (s, 1H), 8.49 (d, J=8.3 Hz, 1H), 8.18 (q, J=8.9 Hz, 2H), 7.67 (d, J=9.2 Hz, 2H), 7.11 (s, 1H), 7.00 (t, J=8.8 Hz, 1H), 6.57 (s, 1H), 6.44 (q, J=5.5 Hz, 2H), 5.99 (d, J=7.7 Hz, 1H), 4.95 (m, 1H), 4.30 (m, 1H), 3.32 (s, 2H), 2.95 (s, 2H), 2.66 (m, 1H), 2.50 (m, 2H), 2.11 (m, 2H), 2.02 (m, 4H), 1.89 (m, 4H), 1.64 (m, 5H), 1.44 (m, 6H), 1.04 (t, J=12.5 Hz, 1H). LC-MS (ES⁺): m/z 302.33 [M+H]⁺.

### Example 2. Compound of Example 2 was Prepared Substantially Following the Synthesis of Example 1

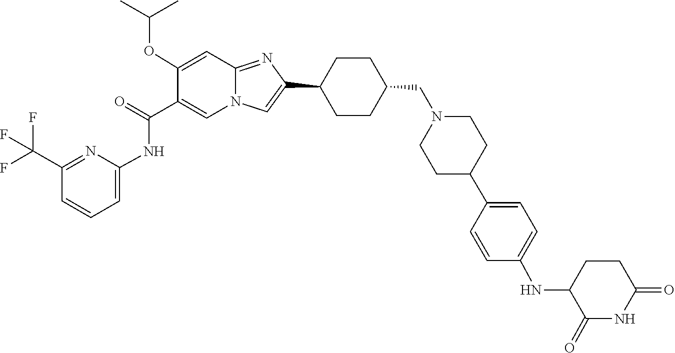 [SMILES: CC(C)Oc1cc2nc([C@H]3CC[C@H](CN4CCC(c5ccc(NC6CCC(=O)NC6=O)cc5)CC4)CC3)cn2cc1C(=O)Nc1cccc(C(F)(F)F)n1]

2-[4-[[4-[4-[(2,6-dioxo-3-piperidyl)amino]phenyl]-1-piperidyl]methyl]cyclohexyl]-7-isopropoxy-N-[6-(trifluoromethyl)-2-pyridyl]imidazo[1,2-a]pyridine-6-carboxamide ¹H NMR (400 MHz, DMSO-d₆) δ 10.98 (s, 1H), 10.77 (s, 1H), 9.11 (s, 1H), 8.49 (d, J=8.4 Hz, 1H), 8.17 (t, J=7.9 Hz, 1H), 7.67 (d, J=8.4 Hz, 1H), 7.11 (s, 1H), 6.96 (d, J=8.4 Hz, 2H), 6.62-6.53 (t, J=17.7 Hz, 2H), 5.65 (d, J=7.4 Hz, 1H), 4.96 (q, J=5.9 Hz, 1H), 4.26 (m, 1H), 3.31 (d, J=9.2 Hz, 2H), 2.99 (d, J=9.2 Hz, 1H), 2.67 (m, 3H), 2.35 (m, 2H), 2.30 (t, J=21.8 Hz, 1H), 2.24 (t, J=21.8 Hz, 2H), 2.08 (m, 5H), 1.87 (m, Hz, 3H), 1.66 (m, 5H), 1.44 (m, 7H), 1.04 (d, J=13.0 Hz, 1H). LC-MS (ES⁺): m/z 746.38 [M+H]⁺.

### Example 3 Compound of Example 3 was Prepared Substantially Following the Synthesis of Example 1

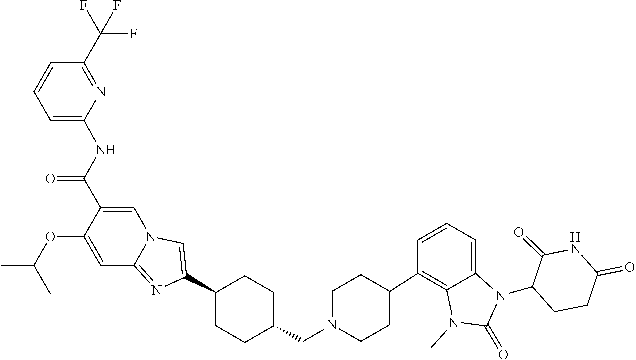 [SMILES: CC(C)Oc1cc2nc([C@H]3CC[C@H](CN4CCC(c5cccc6c5n(C)c(=O)n6C5CCC(=O)NC5=O)CC4)CC3)cn2cc1C(=O)Nc1cccc(C(F)(F)F)n1]

2-[4-[[4-[1-(2,6-dioxo-3-piperidyl)-3-methyl-2-oxo-benzimidazol-4-yl]-1-piperidyl]methyl]cyclohexyl]-7-isopropoxy-N-[6-(trifluoromethyl)-2-pyridyl]imidazo[1,2-a]pyridine-6-carboxamide

¹H NMR (400 MHz, DMSO-d₆) δ 10.98 (s, 1H), 9.11 (s, 1H), 8.46 (m, 4H), 8.17 (t, J=8.0 Hz, 1H), 7.67 (d, J=7.6 Hz, 2H), 7.11 (s, 1H) 7.05 (m, 3H), 5.37 (q, J=5.9 Hz, 1H), 4.95 (t, J=6.0 Hz, 1H), 3.59 (s, 3H), 2.98 (d, J=11.0 Hz, 3H), 2.50 (m, 4H), 2.08-2.07 (m, 2H) 2.00 (m, 7H), 1.77 (m, 4H), 1.44 (m, 8H), 1.23 (m, 1H). LC-MS (ES⁺): m/z 801.41 [M+H]⁺.

### Example 4 Compound of Example 4 was Prepared Substantially Following the Synthesis of Example 1

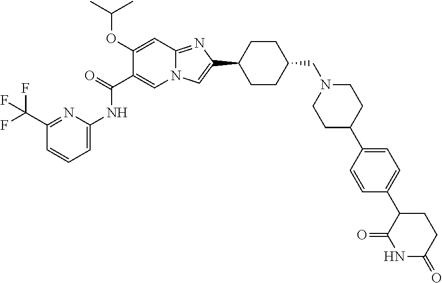 [SMILES: CC(C)Oc1cc2nc([C@H]3CC[C@H](CN4CCC(c5ccc(C6CCC(=O)NC6=O)cc5)CC4)CC3)cn2cc1C(=O)Nc1cccc(C(F)(F)F)n1]

### 2-[4-[[4-[4-(2,6-dioxo-3-piperidyl)phenyl]-1-piperidyl]methyl]cyclohexyl]-7-isopropoxy-N-[6-(trifluoromethyl)-2-pyridyl]imidazo[1,2-a]pyridine-6-carboxamide

¹H NMR (401 MHz, DMSO-d₆) δ 11.04 (s, 1H), 10.83 (s, 1H), 9.13 (s, 1H),), 8.94 (s, 1H), 8.47 (s, 1H), 8.18 (s, 1H), 7.69 (d, J=7.1 Hz, 1H), 7.32-7.10 (m, 2H), 6.97 (s, 1H), 4.96 (s, 1H), 3.83 (s, 1H), 3.62 (s, 2H), 3.03 (m, 7H), 2.13-2.03 (m, 14H), 1.47 (m, 7H), 1.22 (m, 3H). LC-MS (ES⁺): m/z 729.16 [M+H]⁺.

### Example 5 Compound of Example 5 was Prepared Substantially Following the Synthesis of Example 1

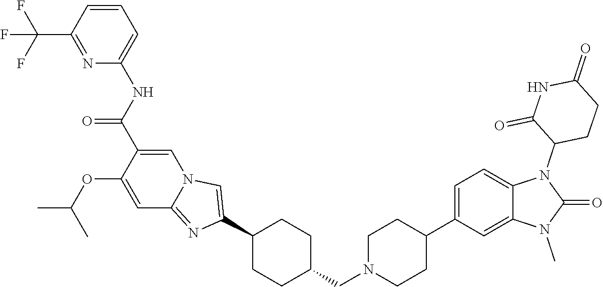 [SMILES: CC(C)Oc1cc2nc([C@H]3CC[C@H](CN4CCC(c5ccc6c(c5)n(C)c(=O)n6C5CCC(=O)NC5=O)CC4)CC3)cn2cc1C(=O)Nc1cccc(C(F)(F)F)n1]

### 2-[4-[[4-[1-(2,6-dioxo-3-piperidyl)-3-methyl-2-oxo-benzimidazol-5-yl]-1-piperidyl]methyl]cyclohexyl]-7-isopropoxy-N-[6-(trifluoromethyl)-2-pyridyl]imidazo[1,2-a]pyridine-6-carboxamide

¹H NMR (400 MHz, DMSO-d₆) δ 11.09 (s, 1H), 10.98 (s, 1H), 9.11 (s, 1H), 8.49 (d, J=8.2 Hz, 1H), 8.17 (t, J=8.0 Hz, 1H), 7.67 (d, J=7.5 Hz, 1H), 7.11 (s, 1H) 7.05 (t, J=26.4 Hz, 2H), 5.37 (d, J=7.2 Hz, 1H), 4.95 (t, J=5.9 Hz, 1H), 3.59 (s, 3H), 2.93-2.64 (m, 6H), 2.63 (m, 4H), 2.32-2.00 (m, 7H), 1.90-1.79 (m, 1H) 1.44-1.43 (m, 8H), 1.23-1.19 (m, 5H), 0.86-0.84 (m, 2H). LC-MS (ES⁺): m/z 801.12 [M+H]⁺.

### Example 6 Compound of Example 6 was Prepared Substantially Following the Synthesis of Example 1

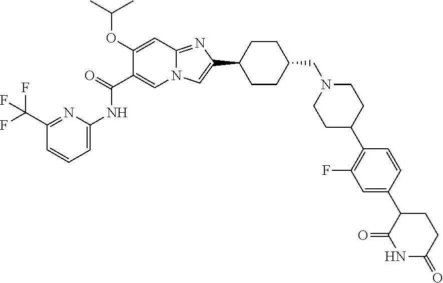 [SMILES: CC(C)Oc1cc2nc([C@H]3CC[C@H](CN4CCC(c5ccc(C6CCC(=O)NC6=O)cc5F)CC4)CC3)cn2cc1C(=O)Nc1cccc(C(F)(F)F)n1]

### 2-[4-[[4-[4-(2,6-dioxo-3-piperidyl)-2-fluoro-phenyl]-1-piperidyl]methyl]cyclohexyl]-7-isopropoxy-N-[6-(trifluoromethyl)-2-pyridyl]imidazo[1,2-a]pyridine-6-carboxamide

¹H NMR (400 MHz, DMSO-d₆) δ10.86 (s, 1H), 9.12 (s, 1H), 9.04 (s, 1H), 8.44 (s, 1H), 8.19 (s, 1H), 7.90 (s, 1H), 7.72 (d, J=7.2 Hz, 1H), 7.31 (s, 1H), 7.10 (d, J=9.6 Hz, 3H), 6.98 (s, 1H), 4.97 (s, 1H), 3.91 (m, 1H), 3.63 (m, 2H), 3.16-3.03 (m, 5H), 2.67 (d, J=45.4 Hz, 1H), 2.08 (m, 11H), 1.53 (m, 8H), 1.38 (m, 3H). LC-MS (ES⁺): m/z 749.32 [M+H]⁺.

### Example 7 Compound of Example 7 was Prepared Substantially Following the Synthesis of Example 1

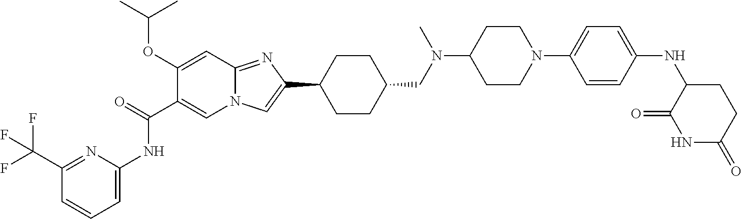 [SMILES: CC(C)Oc1cc2nc([C@H]3CC[C@H](CN(C)C4CCN(c5ccc(NC6CCC(=O)NC6=O)cc5)CC4)CC3)cn2cc1C(=O)Nc1cccc(C(F)(F)F)n1]

### 2-[4-[[[1-[4-[(2,6-dioxo-3-piperidyl)amino]phenyl]-4-piperidyl]-methyl-amino]methyl]cyclohexyl]-7-isopropoxy-N-[6-(trifluoromethyl)-2-pyridyl]imidazo[1,2-a]pyridine-6-carboxamide

¹H NMR (400 MHz, DMSO-d₆) δ 10.98 (s, 1H), 9.61 (d, J=17.1 Hz, 2H), 9.11 (s, 1H), 8.48 (d, J=8.0 Hz, 1H), 8.19 (q, J=12.2 Hz, 1H), 7.68 (d, J=6.3 Hz, 2H), 7.10 (m, 10H), 6.78 (d, J=7.4 Hz, 2H), 6.61 (d, J=8.3 Hz, 2H), 4.96 (q, J=6.0 Hz, 1H), 4.20 (s, 1H), 3.62-3.57 (m, 4H), 3.10 (t, J=27.7 Hz, 2H), 2.94-2.84 (m, 3H), 1.92 (m, 6H), 1.44 (m, 7H), 1.25-0.85 (m, 3H). LC-MS (ES⁺): m/z 775.37 [M+H]⁺.

### Example 8 Compound of Example 8 was Prepared Substantially Following the Synthesis of Example 1

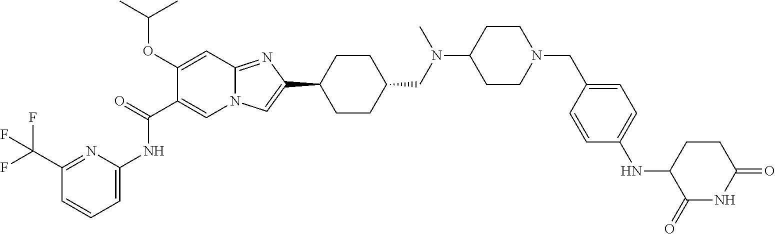 [SMILES: CC(C)Oc1cc2nc([C@H]3CC[C@H](CN(C)C4CCN(Cc5ccc(NC6CCC(=O)NC6=O)cc5)CC4)CC3)cn2cc1C(=O)Nc1cccc(C(F)(F)F)n1]

### 2-[4-[[[1-[[4-[(2,6-dioxo-3-piperidyl)amino]phenyl]methyl]-4-piperidyl]-methyl-amino]methyl]cyclohexyl]-7-isopropoxy-N-[6-(trifluoromethyl)-2-pyridyl]imidazo[1,2-a]pyridine-6-carboxamide

¹H NMR (400 MHz, DMSO-d₆) δ 11.11 (s, 1H), 10.82 (s, 1H), 9.15 (s, 1H), 8.47 (s, 1H), 8.18 (t, J=7.6 Hz, 1H), 7.75 (t, J=23.1 Hz, 1H), 7.10 (m, J=25.5 Hz, 7H), 6.73 (d, J=7.8 Hz, 3H), 6.24 (s, 1H), 4.97 (s, 1H), 4.37 (s, 1H), 4.14 (s, 2H), 2.79 (q, J=44.3 Hz, 4H), 2.06 (m, 16H), 1.45 (m, 11H). LC-MS (ES⁺): m/z 789.19 [M+H]⁺.

### Example 9 Compound of Example 9 was Prepared Substantially Following the Synthesis of Example 1

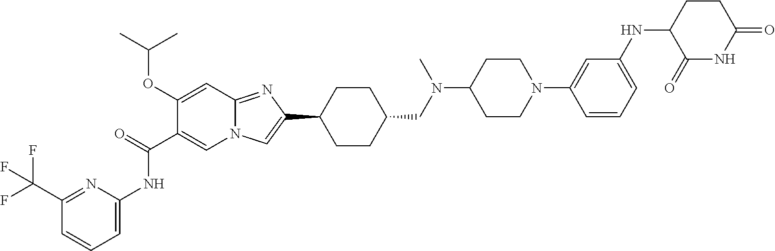 [SMILES: CC(C)Oc1cc2nc([C@H]3CC[C@H](CN(C)C4CCN(c5cccc(NC6CCC(=O)NC6=O)c5)CC4)CC3)cn2cc1C(=O)Nc1cccc(C(F)(F)F)n1]

### 2-[4-[[[1-[3-[(2,6-dioxo-3-piperidyl)amino]phenyl]-4-piperidyl]-methyl-amino]methyl]cyclohexyl]-7-isopropoxy-N-[6-(trifluoromethyl)-2-pyridyl]imidazo[1,2-a]pyridine-6-carboxamide

¹H NMR (400 MHz, DMSO-d₆) δ 10.75 (d, J=6.4 Hz, 1H), 9.09 (d, J=5.1 Hz, 1H), 8.49 (d, J=8.6 Hz, 1H), 8.29 (s, 2H), 8.17 (t, J=8.0 Hz, 1H), 7.67 (t, J=5.4 Hz, 1H), 7.12 (d, J=12.1 Hz, 1H), 6.90 (t, J=7.8 Hz, 1H), 6.60 (s, 1H), 6.18-6.10 (m, 3H), 5.60 (t, J=7.1 Hz, 1H), 4.95 (t, J=5.9 Hz, 1H), 4.28 (d, J=12.1 Hz, 1H), 3.66 (d, J=9.8 Hz, 2H), 2.50 (m, 5H), 2.32 (m, 1H) 2.21 (m, 5H), 2.06 (d, J=10.2 Hz, 2H), 1.89 (m, 3H), 1.72 (m, 3H), 1.43 (m, 8H), 1.00-0.84 (m, 4H). LC-MS (ES⁺): m/z 775.20 [M+H]⁺.

### Example 10 Compound of Example 10 was Prepared Substantially Following the Synthesis of Example 1

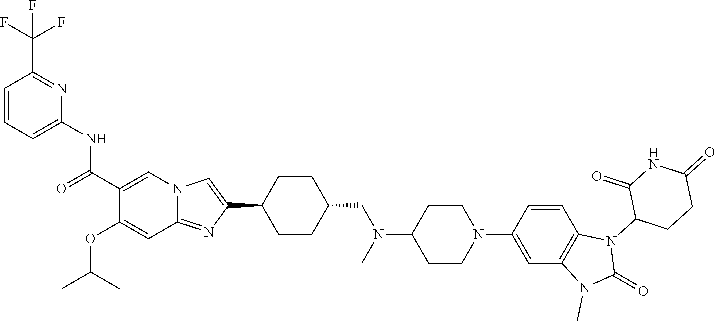 [SMILES: CC(C)Oc1cc2nc([C@H]3CC[C@H](CN(C)C4CCN(c5ccc6c(c5)n(C)c(=O)n6C5CCC(=O)NC5=O)CC4)CC3)cn2cc1C(=O)Nc1cccc(C(F)(F)F)n1]

### 2-[4-[[[1-[1-(2,6-dioxo-3-piperidyl)-3-methyl-2-oxo-benzimidazol-5-yl]-4-piperidyl]-methyl-amino]methyl]cyclohexyl]-7-isopropoxy-N-[6-(trifluoromethyl)-2-pyridyl]imidazo[1,2-a]pyridine-6-carboxamide

¹H NMR (400 MHz, DMSO-d₆) δ 11.85 (s, 1H), 9.08 (s, 1H), 8.41 (t, J=21.5 Hz, 1H), 8.34 (t, J=8.0 Hz, 1H), 8.14 (t, J=8.0 Hz, 1H), 7.68 (d, J=8.1 Hz, 1H), 7.41 (q, J=34.5 Hz, 1H), 7.02 (t, J=13.9 Hz, 1H), 6.87 (d, J=8.7 Hz, 1H), 6.73 (d, J=8.2 Hz, 1H), 6.62 (d, J=8.7 Hz, 1H), 5.25 (q, J=5.9 Hz, 1H), 4.94 (t, J=6.0 Hz, 1H), 3.73 (d, J=10.6 Hz, 2H), 3.47 (d, J=4.5 Hz, 3H), 3.32 (d, J=6.8 Hz, 1H), 3.15 (m, 3H), 2.92 (d, J=6.8 Hz, 1H), 2.73 (m, 2H), 2.54 (m, 8H), 2.00 (m, 1H), 2.84 (m, 2H), 1.76 (m, 2H), 1.561.61 (m, 8H), 1.38-1.44 (m, 2H), 0.96 (m, 2H). LC-MS (ES⁺): m/z 830.14 [M+H]⁺.

### Example 11 Compound of Example 11 was Prepared Substantially Following the Synthesis of Example 1

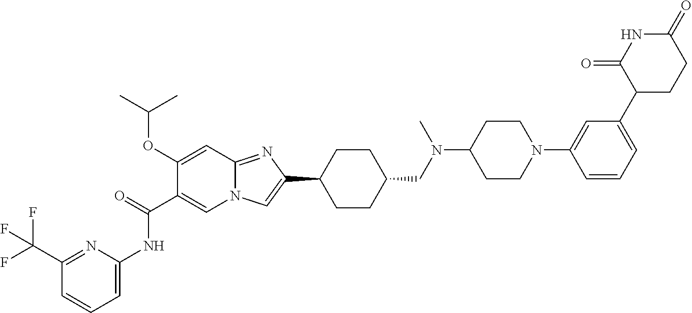 [SMILES: CC(C)Oc1cc2nc([C@H]3CC[C@H](CN(C)C4CCN(c5cccc(C6CCC(=O)NC6=O)c5)CC4)CC3)cn2cc1C(=O)Nc1cccc(C(F)(F)F)n1]

### 2-[4-[[[1-[3-(2,6-dioxo-3-piperidyl)phenyl]-4-piperidyl]-methyl-amino]methyl]cyclohexyl]-7-isopropoxy-N-[6-(trifluoromethyl)-2-pyridyl]imidazo[1,2-a]pyridine-6-carboxamide

1H NMR (400 MHz, DMSO-d₆) δ 10.99 (s, 1H), 10.80 (d, J=5.7 Hz, 1H), 9.12 (s, 1H), 8.83 (s, 1H), 8.48 (d, J=8.4 Hz, 1H), 8.16 (q, J=7.5 Hz, 1H), 7.68 (t, J=3.7 Hz, 2H), 7.14 (q, J=11.0 Hz, 2H), 6.84 (q, J=8.8 Hz, 2H), 6.64 (d, J=7.1 Hz, 1H), 6.52 (s, 1H), 4.95 (m, 1H), 3.85 (s, 3H), 2.50 (m, 9H), 1.97 (m, 12H), 1.51 (m, 8H), 1.44 (m, 1H). LC-MS (ES⁺): m/z 760.16 [M+H]⁺.

### Example 12 Compound of Example 12 was Prepared Substantially Following the Synthesis of Example 1

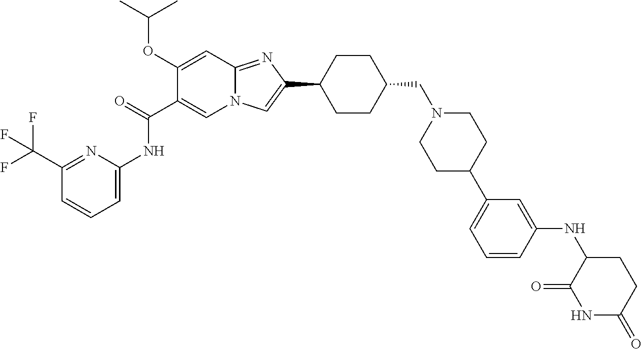 [SMILES: CC(C)Oc1cc2nc([C@H]3CC[C@H](CN4CCC(c5cccc(NC6CCC(=O)NC6=O)c5)CC4)CC3)cn2cc1C(=O)Nc1cccc(C(F)(F)F)n1]

### 2-[4-[[4-[3-[(2,6-dioxo-3-piperidyl)amino]phenyl]-1-piperidyl]methyl]cyclohexyl]-7-isopropoxy-N-[6-(trifluoromethyl)-2-pyridyl]imidazo[1,2-a]pyridine-6-carboxamide

¹H NMR (400 MHz, DMSO-d₆) δ 10.76 (s, 1H), 9.10 (d, J=5.2 Hz, 1H), 8.49 (d, J=8.3 Hz, 1H), 8.36 (s, 2H), 8.17 (t, J=8.0 Hz, 1H), 7.70 (t, J=14.9 Hz, 2H), 7.13-6.44 (m, 7H), 5.71 (d, J=7.4 Hz, 1H), 4.96 (q, J=5.8 Hz, 1H), 4.33 (s, 1H), 2.75 (m, 2H), 2.60 (m, 1H), 2.55 (m, 4H), 2.33 (s, 1H), 2.06 (m, 4H), 1.80 (m, 8H), 1.75-1.43 (m, 5H), 1.02 (d, J=12.2 Hz, 1H). LC-MS (ES⁺): m/z 746. 17 [M+H]⁺. Example 13 Compound of Example 13 was Prepared Substantially Following the Synthesis of Example 1

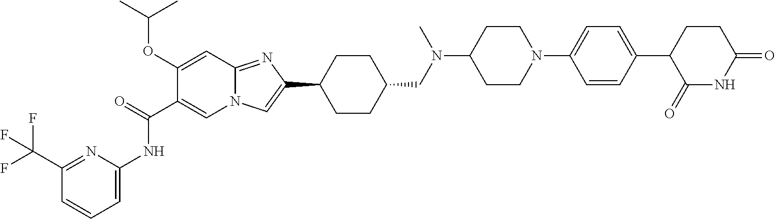 [SMILES: CC(C)Oc1cc2nc([C@H]3CC[C@H](CN(C)C4CCN(c5ccc(C6CCC(=O)NC6=O)cc5)CC4)CC3)cn2cc1C(=O)Nc1cccc(C(F)(F)F)n1]

### 2-[4-[[[1-[4-(2,6-dioxo-3-piperidyl)phenyl]-4-piperidyl]-methyl-amino]methyl]cyclohexyl]-7-isopropoxy-N-[6-(trifluoromethyl)-2-pyridyl]imidazo[1,2-a]pyridine-6-carboxamide

¹H NMR (400 MHz, DMSO-d₆) δ 10.98 (s, 1H), 10.76 (d, J=4.6 Hz, 1H), 9.10 (d, J=3.6 Hz, 1H), 8.48 (d, J=8.4 Hz, 1H), 8.15 (d, J=9.3 Hz, 2H), 7.69 (t, J=14.0 Hz, 2H), 7.13 (m, 1H), 7.08 (m, 1H), 6.89 (t, J=8.3 Hz, 2H), 6.52 (s, 5H), 4.95 (m, 1H), 3.72 (q, J=5.3 Hz, 3H), 2.63 (m, 4H), 2.36 (q, J=10.1 Hz, 3H), 1.97 (m, J=10.4 Hz, 4H), 1.56 (d, J=10.2 Hz, 4H), 1.43 (d, J=6.0 Hz, 8H), 1.04 (t, J=11.9 Hz, 2H), 0.09 (s, 2H). LC-MS (ES⁺): m/z 760.16 [M+H]⁺.

### Example 14 Compound of Example 14 was Prepared Substantially Following the Synthesis of Example 1

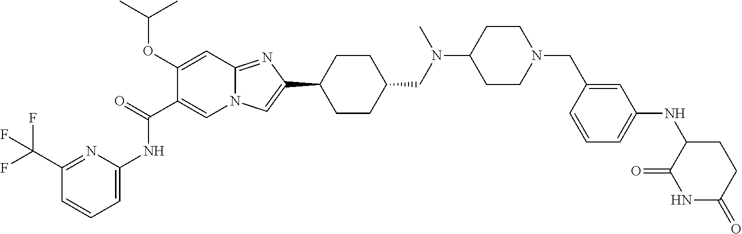 [SMILES: CC(C)Oc1cc2nc([C@H]3CC[C@H](CN(C)C4CCN(Cc5cccc(NC6CCC(=O)NC6=O)c5)CC4)CC3)cn2cc1C(=O)Nc1cccc(C(F)(F)F)n1]

### 2-[4-[[[1-[[3-[(2,6-dioxo-3-piperidyl)amino]phenyl]methyl]-4-piperidyl]-methyl-amino]methyl]cyclohexyl]-7-isopropoxy-N-[6-(trifluoromethyl)-2-pyridyl]imidazo[1,2-a]pyridine-6-carboxamide

¹H NMR (400 MHz, DMSO-d₆) δ 11.20 (s, 1H), 10.85 (s, 1H), 9.72 (s, 1H), 8.47 (s, 1H), 8.19 (t, J=7.8 Hz, 1H), 7.92 (d, J=16.1 Hz, 1H), 7.71 (d, J=7.4 Hz, 1H), 7.29 (s, 1H), 7.19 (m, 5H), 6.72 (t, J=13.6 Hz, 3H), 6.11 (s, 1H), 4.99 (d, J=5.4 Hz, 1H), 4.33 (d, J=6.7 Hz, 1H), 4.18 (s, 2H), 2.97 (s, 5H), 2.75 (m, 6H), 2.10 (m, 7H), 1.94 (m, 8H), 1.38 (d, J=17.2 Hz, 3H), 1.19 (d, J=36.1 Hz, 1H). LC-MS (ES⁺): m/z 789.15 [M+H]⁺.

### Synthesis of 6-(difluoromethyl)pyridin-2-amine

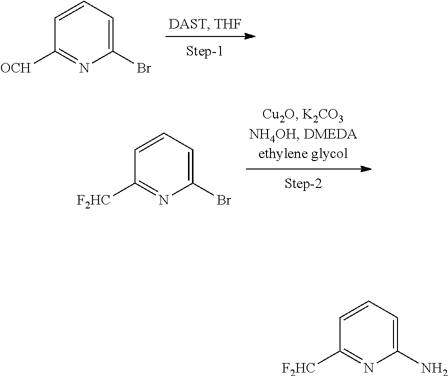

### Step-1:

To a stirred solution of 6-bromopyridine-2-carbaldehyde (25 g, 134.40 mmol) in DCM (500 mL) was added diethylaminosulfur trifluoride (36.60 g, 227.06 mmol, 30 mL) dropwise at 0° C. over a period of 20 minutes. The reaction mixture was stirred at this temperature for 2 hours and the progress of reaction was monitored by LCMS and TLC. Upon completion, the reaction was quenched with NaHCO₃ solution (2×250 mL) and extracted with DCM (3×250 mL). The combined organic layers were dried over anhydrous Na₂SO₄ and concentrated in vacuo to give the crude product, which was purified by column chromatography (silica gel 100-200 mesh, 30-100% ethyl acetate in pet ether) to afford 2-bromo-6-(difluoromethyl)pyridine (11.0 g, 51.83 mmol, 38.56% yield) as a gummy liquid. ¹H NMR (400 MHz, CDCl₃) δ: 7.71 (t, J=7.73 Hz, 1H), 7.62 (d, J=7.54 Hz, 2H), 6.59 (t, J=55.11 Hz, 1H).

### Step-2:

To a stirred solution of 2-bromo-6-(difluoromethyl)pyridine (12.0 g, 57.69 mmol, 7.06 mL) in ethylene glycol (150 mL) were added copper(I) oxide (0.620 g, 4.33 mmol), potassium carbonate, anhydrous, 99% (0.750 g, 5.43 mmol) and 1,1-dimethylethylene diamine (5.09 g, 57.69 mmol). Aqueous ammonia (57.69 mmol, 120 mL) was then added dropwise at room temperature over 10 minutes. The reaction mixture was heated at 120° C. for 16 hours and the progress of reaction was monitored by LC-MS and TLC. Upon completion, the reaction was quenched with NaHCO₃ solution and extracted with DCM (3×250 mL). The combined organic layers were dried over anhydrous Na₂SO₄ and concentrated in vacuo to give the crude compound, which was purified by column chromatography (silica gel 100-200 mesh, 30-100% ethyl acetate in pet ether) to afford 6-(difluoromethyl)pyridin-2-amine (7.2 g, 48.96 mmol, 84.86% yield). LC-MS (ES⁺): m/z 145.20 [M+H]⁺.

### Synthesis of 6-(difluoromethyl)-N-[2-[4-(hydroxymethyl)cyclohexyl]-7-isopropoxy-imidazo [1,2-a] pyridin-6-yl] pyridine-2-carboxamide

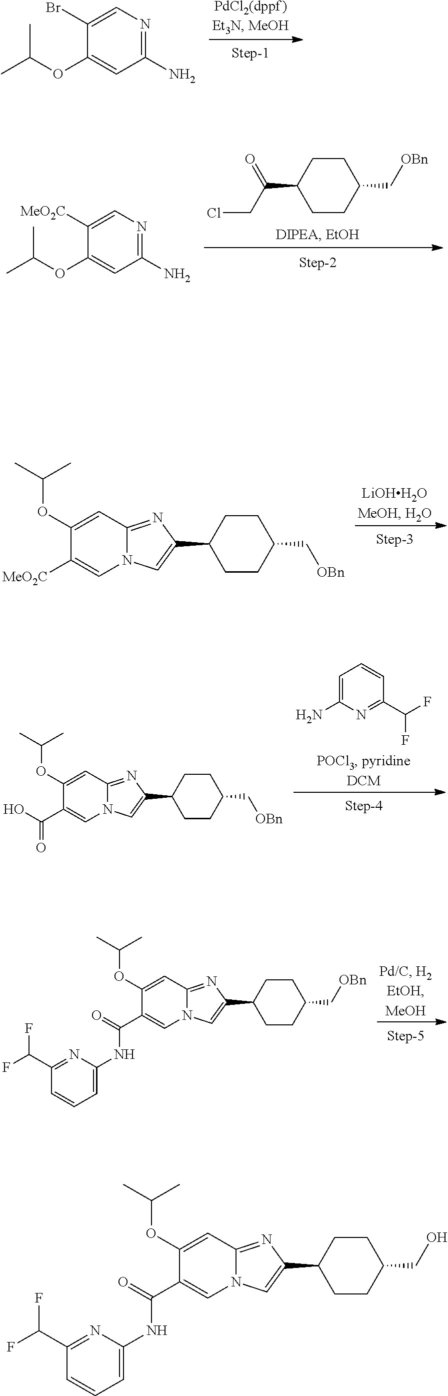

### Step-1:

Under an atmosphere of N₂, a pressure bomb was charged with 5-bromo-4-isopropoxy-pyridin-2-amine (20 g, 86.55 mmol), triethylamine (72.60 g, 717.46 mmol, 100 mL) and Pd(dppf)Cl₂ (6.33 g, 8.65 mmol) in methanol (400 mL). The reaction mixture was degassed with N₂ for 15 minutes, before the pressure bomb was sealed with carbon monoxide (600 Psi). The resulting reaction mixture was stirred at 100° C. for 36 hours and the progress of the reaction was monitored by TLC and LCMS. After completion of the reaction, the reaction mixture was filtered through celite bed and washed with ethyl acetate (500 mL). The combined organic layers were concentrated under reduced pressure and purified by column chromatography (silica gel 100-200 mesh, 0-40% ethyl acetate in pet ether) to afford methyl 6-amino-4-isopropoxy-pyridine-3-carboxylate (7.2 g, 32.36 mmol, 37.39% yield) as a yellow solid. LC-MS (ES⁺): m/z 211.46 [M+H]⁺.

### Step-2:

In a sealed tube, a solution of methyl 6-amino-4-isopropoxy-pyridine-3-carboxylate (20 g, 95.13 mmol), 1-[4-(benzyloxymethyl)cyclohexyl]-2-chloro-ethanone (46.13 g, 164.30 mmol) and DIPEA (35.62 g, 275.58 mmol, 48.00 mL) was heated at 95° C. with stirring for 16 hours. The progress of the reaction was monitored by LCMS/TLC. After complete conversion of the starting material, the reaction mixture was concentrated under reduced pressure and the resulting crude product was purified by column chromatography (0-50% ethyl acetate in pet ether) to afford methyl 2-[4-(benzyloxymethyl)cyclohexyl]-7-isopropoxy-imidazo[1,2-a]pyridine-6-carboxylate (15 g, 31.96 mmol, 33.59% yield) as a yellow gum. LC-MS (ES⁺): m/z 437.52 [M+H]⁺.

### Step-3:

To a stirred solution of methyl 2-[4-(benzyloxymethyl)cyclohexyl]-7-isopropoxy-imidazo[1,2-a]pyridine-6-carboxylate (0.05 g, 114.54 μmol) in water (999.35 μL) methanol (3 mL) cooled to 0-5° C., lithium hydroxide monohydrate, 98% (4.81 mg, 114.54 μmol, 3.18 L) was added at 5° C. stirred for 4 hours. Progress of the reaction was monitored by LC-MS. After completion of the reaction, the reaction mass was concentrated and the crude compound was dissolved in water and wash with ethyl acetate (150 ml×2). The aqueous layer was acidified with 2N HCL to pH=5-6 and extracted with 10% methanol in DCM (500 ml×2). The organic layer was then dried over anhydrous sodium sulfate and concentrated to afford the product 2-[4-(benzyloxymethyl)cyclohexyl]-7-isopropoxy-imidazo[1,2-a]pyridine-6-carboxylic acid (0.05 g, 108.75 μmol, 94.95% yield). LC-MS (ES⁺): m/z 423.49 [M+H]⁺.

### Step-4:

To a stirred solution of 2-[4-(benzyloxymethyl)cyclohexyl]-7-isopropoxy-imidazo[1,2-a]pyridine-6-carboxylic acid (3.5 g, 8.28 mmol) and 6-(difluoromethyl)pyridin-2-amine (1.2 g, 8.33 mmol) in DCM (100 mL) was added pyridine (11.74 g, 148.37 mmol, 12 mL) dropwise at 0° C. and followed by phosphorus oxychloride (3.81 g, 24.85 mmol, 1.8 mL) and the reaction was stirred for 4 hours at 15° C. After completion of the reaction, the reaction mixture was cooled to room temperature, added water, and extracted with ethyl acetate. The combined organic phase was washed with brine solution and the organic layer was dried over sodium sulfate, filtered, and concentrated under reduced pressure. The crude product was purified by column chromatography (100-200 mesh silica gel, 0-30% ethyl acetate in pet ether) to afford the product 2-[4-(benzyloxymethyl)cyclohexyl]-N-[6-(difluoromethyl)-2-pyridyl]-7-isopropoxy-imidazo[1,2-a]pyridine-6-carboxamide (2.5 g, 4.47 mmol, 53.91% yield) as a yellow solid. LC-MS (ES⁻): m/z 547.26 [M−H]⁻.

### Step-5:

A stirred solution of 2-[4-(benzyloxymethyl)cyclohexyl]-N-[6-(difluoromethyl)-2-pyridyl]-7-isopropoxy-imidazo[1,2-a]pyridine-6-carboxamide (3.00 g, 5.47 mmol) in ethanol (50 mL) and methanol (50 mL) was purged with hydrogen gas followed by the addition of palladium, 10% on carbon, type 487, dry (1.57 g, 14.76 mmol) and concentrated HCl (191.39 mg, 5.47 mmol, 1.0 mL). The reaction was stirred under hydrogen atmosphere (1 atm) at room temperature for 5 hours. The progress of the reaction monitored by LC-MS. After completion of the reaction, the reaction mixture was filtered through celite bed and washed with methanol (50 mL×2) and the organic layer was concentrated. The crude compound was dissolved in saturated bicarbonate solution and extracted with 10% methanol in DCM. The organic layer was washed with brine solution, dried over sodium sulfate, and concentrated in vacuo. The crude compound was purified by column chromatography (silica gel, 0-10% MeOH in DCM) to afford the product N-[6-(difluoromethyl)-2-pyridyl]-2-[4-(hydroxymethyl)cyclohexyl]-7-isopropoxy-imidazo[1,2-a]pyridine-6-carboxamide (2 g, 3.97 mmol, 72.59% yield) as a yellow solid. LC-MS (ES⁻): m/z 457.26 [M−H]⁻.

### Example 15 Synthesis of N-[6-(difluoromethyl)-2-pyridyl]-2-[4-[[4-[4-(2,6-dioxo-3-piperidyl)phenyl]-1-piperidyl]methyl]cyclohexyl]-7-isopropoxy-imidazo[1,2-a]pyridine-6-carboxamide

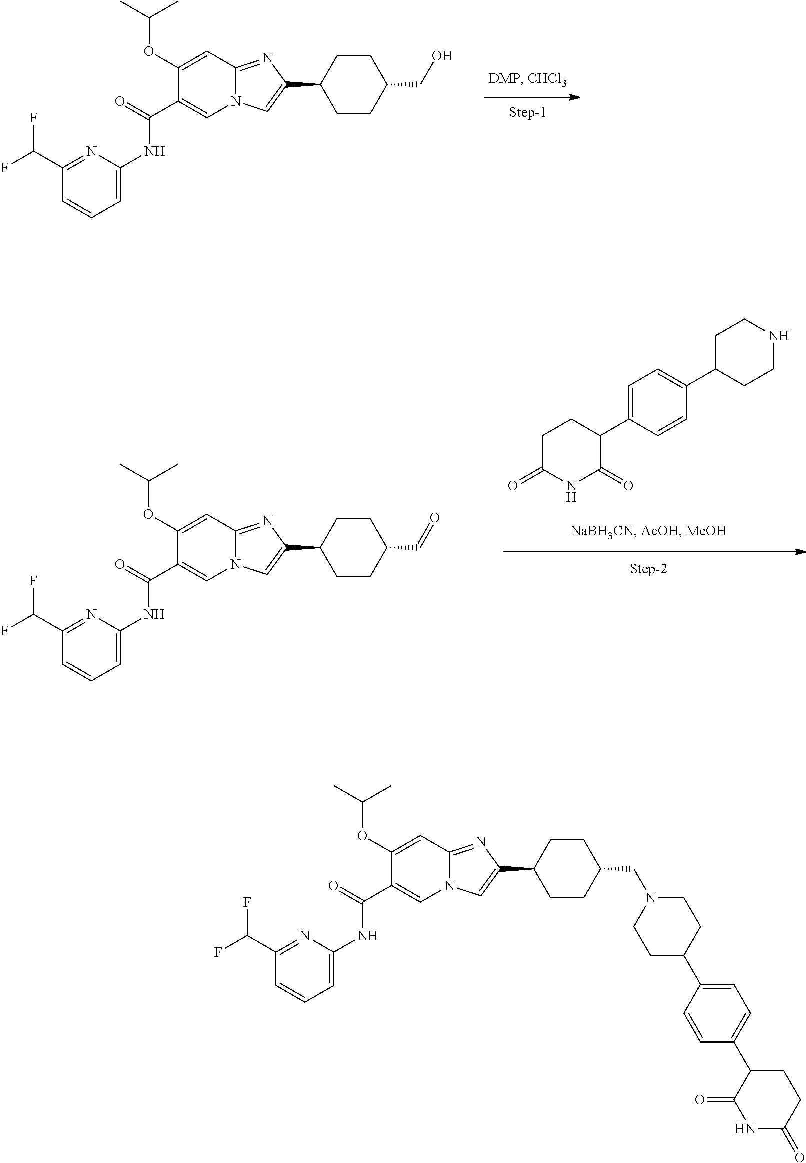 [SMILES: CC(C)Oc1cc2nc([C@H]3CC[C@H](CO)CC3)cn2cc1C(=O)Nc1cccc(C(F)F)n1 ; CC(C)Oc1cc2nc([C@H]3CC[C@H](C=O)CC3)cn2cc1C(=O)Nc1cccc(C(F)F)n1 ; O=C1CCC(c2ccc(C3CCNCC3)cc2)C(=O)N1 ; CC(C)Oc1cc2nc([C@H]3CC[C@H](CN4CCC(c5ccc(C6CCC(=O)NC6=O)cc5)CC4)CC3)cn2cc1C(=O)Nc1cccc(C(F)F)n1]

### Step-1:

To a stirred solution of N-[6-(difluoromethyl)-2-pyridyl]-2-[4-(hydroxymethyl) cyclohexyl]-7-isopropoxy-imidazo[1,2-a]pyridine-6-carboxamide (1 g, 2.18 mmol) in chloroform (25 mL) was added Dess-Martin Periodinane (1.5 g, 3.54 mmol) at 0-5° C. The reaction was then warmed up to 25° C. and stirred for an additional 4 hours. Progress of the reaction was monitored by LCMS/TLC. After completion of the reaction, the reaction was quenched with cold saturated sodium bicarbonate solution, extracted with ethyl acetate. The organic layer was washed with sodium bicarbonate solution, brine, dried over anhydrous Na₂SO₄ and concentrated in vacuo to afford the product N-[6-(difluoromethyl)-2-pyridyl]-2-(4-formylcyclohexyl)-7-isopropoxy-imidazo[1,2-a]pyridine-6-carboxamide (0.8 g, 1.54 mmol, 70.71% yield) as a yellow solid. LC-MS (ES⁺): m/z 457.47 [M+H]⁺.

### Step-2:

To a stirred solution of N-[6-(difluoromethyl)-2-pyridyl]-2-(4-formylcyclohexyl)-7-isopropoxy-imidazo[1,2-a]pyridine-6-carboxamide (80.00 mg, 175.25 μmol), 3-[4-(4-piperidyl)phenyl]piperidine-2,6-dione TFA salt (47.73 mg, 123.53 μmol) in methanol (3 mL) was added acetic acid (1.05 mg, 17.53 μmol, 1.00 μL) and the reaction mixture was heated to 60° C. for 2 hours. The reaction mixture was then cooled to room temperature and sodium cyanoborohydride (22.03 mg, 350.51 μmol) was added and stirred for 16 hours. The reaction progress was monitored by LCMS. Upon completion of the reaction, the reaction mixture was concentrated in vacuo and purified by prep-HPLC to afford N-[6-(difluoromethyl)-2-pyridyl]-2-[4-[[4-[4-(2,6-dioxo-3-piperidyl)phenyl]-1-piperidyl]methyl] cyclohexyl]-7-isopropoxy-imidazo[1,2-a]pyridine-6-carboxamide TFA salt (26 mg, 29.88 μmol, 17.05% yield). ¹H NMR (400 MHz, DMSO-d₆) δ 11.03 (s, 1H), 10.84 (s, 1H), 9.17 (s, 1H), 8.86 (s, 1H), 8.35 (s, 1H), 8.09 (t, J=7.9 Hz, 1H), 7.88 (s, 1H), 7.50 (d, J=7.7 Hz, 1H), 7.49 (m, 1H), 7.21 (m, 5H), 6.99 (q, J=23.3 Hz, 2H), 5.01 (s, 1H), 3.84 (q, J=5.5 Hz, 3H), 3.63 (d, J=10.8 Hz, 2H), 3.06 (m, 3H), 2.82 (m, 7H), 2.52 (mz, 2H), 2.05 (m, 2H), 1.48 (m, 6H), 1.22 (m, 3H), 0.85 (m, 1H). LC-MS (ES⁺): m/z 713.43 [M+H]⁺.

### Example 16 Compound of Example 16 was Prepared Substantially Following the Synthesis of Example 15

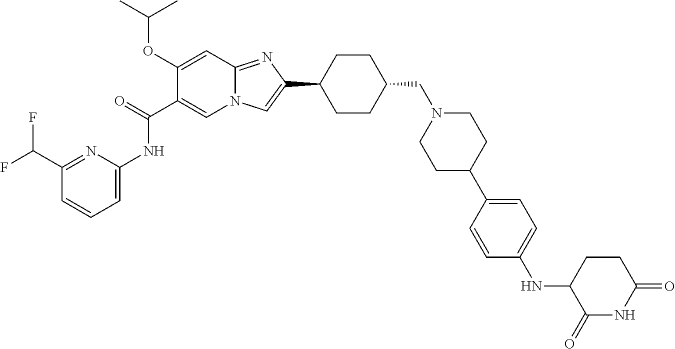 [SMILES: CC(C)Oc1cc2nc([C@H]3CC[C@H](CN4CCC(c5ccc(NC6CCC(=O)NC6=O)cc5)CC4)CC3)cn2cc1C(=O)Nc1cccc(C(F)F)n1]

### N-[6-(difluoromethyl)-2-pyridyl]-2-[4-[[4-[4-[(2,6-dioxo-3-piperidyl)amino]phenyl]-1-piperidyl]methyl]cyclohexyl]-7-isopropoxy-imidazo[1,2-a]pyridine-6-carboxamide

¹H NMR (400 MHz, DMSO-d₆) δ 11.07 (s, 1H), 10.78 (s, 1H), 9.18 (s, 1H), 9.05 (s, 1H), 8.34 (s, 1H), 8.09 (t, J=7.8 Hz, 1H), 7.93 (s, 1H), 7.51 (d, J=7.3 Hz, 1H), 7.35 (s, 2H), 7.01 (q, J=26.6 Hz, 3H), 6.65 (d, J=8.2 Hz, 1H), 5.00 (s, 1H), 4.28 (q, J=5.3 Hz, 1H), 3.5 (s, 2H), 3.02 (s, 4H), 2.72 (m, 4H), 2.11 (m, 3H), 1.92 (m, 8H), 1.47 (m, 8H), 1.21 (t, J=9.3 Hz, 2H). LC-MS (ES⁺): m/z 728.39 [M+H]⁺.

### Example 17 Compound of Example 17 was Prepared Substantially Following the Synthesis of Example 15

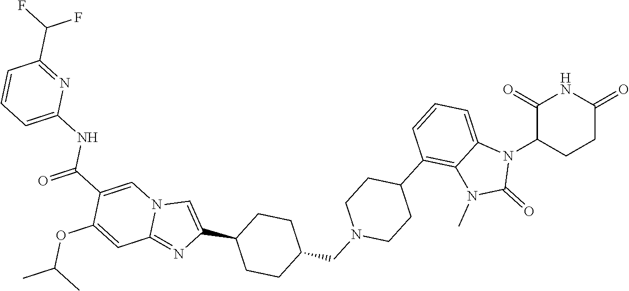 [SMILES: CC(C)Oc1cc2nc([C@H]3CC[C@H](CN4CCC(c5cccc6c5n(C)c(=O)n6C5CCC(=O)NC5=O)CC4)CC3)cn2cc1C(=O)Nc1cccc(C(F)F)n1]

### N-[6-(difluoromethyl)-2-pyridyl]-2-[4-[[4-[1-(2,6-dioxo-3-piperidyl)-3-methyl-2-oxo-benzimidazol-4-yl]-1-piperidyl]methyl]cyclohexyl]-7-isopropoxy-imidazo[1,2-a]pyridine-6-carboxamide

¹H NMR (400 MHz, DMSO-d₆) δ 11.09 (s, 1H), 10.86 (s, 1H), 9.11 (s, 1H), 8.37 (d, J=7.9 Hz, 1H), 8.05 (q, J=10.6 Hz, 1H), 7.66 (s, 1H), 7.47 (d, J=7.5 Hz, 1H), 7.11 (s, 1H), 7.00 (q, J=6.4 Hz, 3H), 6.82 (d, J=54.9 Hz, 1H), 5.37 (q, J=5.8 Hz, 1H), 4.95 (t, J=6.0 Hz, 1H), 3.59 (s, 3H), 2.95 (m, 4H), 2.67 (m, 3H), 2.19 (d, J=7.1 Hz, 2H), 1.99 (m, 7H), 1.79 (m, 4H), 1.58 (s, 1H), 1.45 (m, 8H), 1.28 (m, 1H), 1.04 (m, 2H). LC-MS (ES⁺): m/z 783.39 [M+H]⁺.

### Example 18 Compound of Example 18 was Prepared Substantially Following the Synthesis of Example 15

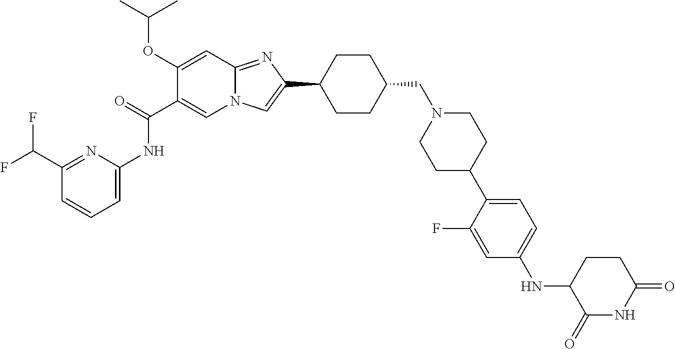 [SMILES: CC(C)Oc1cc2nc([C@H]3CC[C@H](CN4CCC(c5ccc(NC6CCC(=O)NC6=O)cc5F)CC4)CC3)cn2cc1C(=O)Nc1cccc(C(F)F)n1]

### N-[6-(difluoromethyl)-2-pyridyl]-2-[4-[[4-[4-[(2,6-dioxo-3-piperidyl)amino]-2-fluoro-phenyl]-1-piperidyl]methyl]cyclohexyl]-7-isopropoxy-imidazo[1,2-a]pyridine-6-carboxamide

¹H NMR (400 MHz, DMSO-d₆) δ 11.06 (s, 1H), 10.80 (s, 1H), 9.18 (s, 1H), 8.95 (s, 1H), 8.35 (s, 1H), 8.09 (t, J=7.9 Hz, 1H), 7.91 (s, 1H), 7.50 (d, J=7.4 Hz, 1H), 7.21-6.77 (m, 3H), 6.49 (t, J=8.2 Hz, 2H), 6.12 (s, 1H), 5.01 (s, 1H), 4.33 (d, J=9.0 Hz, 1H), 3.5 (m, 1H), 3.0 (m, 8H), 2.86 (m, 11H), 2.01 (m, 8H), 1.34 (m, 2H). LC-MS (ES⁺): m/z 746.41 [M+H]⁺.

### Example 19 Compound of Example 19 was Prepared Substantially Following the Synthesis of Example 15

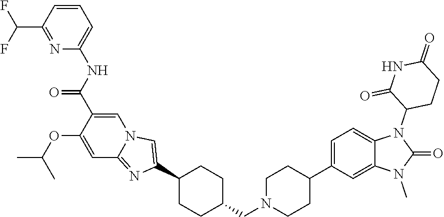 [SMILES: CC(C)Oc1cc2nc([C@H]3CC[C@H](CN4CCC(c5ccc6c(c5)n(C)c(=O)n6C5CCC(=O)NC5=O)CC4)CC3)cn2cc1C(=O)Nc1cccc(C(F)F)n1]

### N-[6-(difluoromethyl)-2-pyridyl]-2-[4-[[4-[1-(2,6-dioxo-3-piperidyl)-3-methyl-2-oxo-benzimidazol-5-yl]-1-piperidyl]methyl]cyclohexyl]-7-isopropoxy-imidazo[1,2-a]pyridine-6-carboxamide

¹H NMR (400 MHz, DMSO-d₆) δ 11.11 (s, 1H), 10.95 (s, 1H), 9.15 (s, 1H), 8.85 (s, 1H), 8.35 (s, 1H), 8.09 (t, J=7.9 Hz, 1H), 7.49 (d, J=7.4 Hz, 1H), 7.20-6.76 (m, 5H), 6.52 (s, 1H), 5.39 (q, J=6.1 Hz, 1H), 4.99 (s, 1H), 3.63 (d, J=4.1 Hz, 6H), 2.88 (d, J=11.9 Hz, 2H), 2.69 (m, 3H), 2.03-2.11 (m, 11H), 1.94 (m, 1H), 1.47 (m, 8H), 1.21 (m, 2H). LC-MS (ES⁺): m/z 783.26 [M+H]⁺.

### Example 20 Compound of Example 20 was Prepared Substantially Following the Synthesis of Example 15

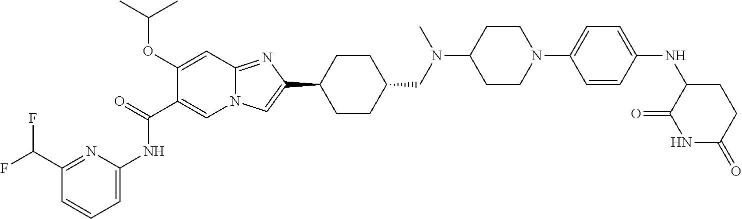 [SMILES: CC(C)Oc1cc2nc([C@H]3CC[C@H](CN(C)C4CCN(c5ccc(NC6CCC(=O)NC6=O)cc5)CC4)CC3)cn2cc1C(=O)Nc1cccc(C(F)F)n1]

### N-[6-(difluoromethyl)-2-pyridyl]-2-[4-[[[1-[4-[(2,6-dioxo-3-piperidyl)amino]phenyl]-4-piperidyl]-methyl-amino]methyl]cyclohexyl]-7-isopropoxy-imidazo[1,2-a]pyridine-6-carboxamide

¹H NMR (400 MHz, DMSO-d₆) δ 11.07 (s, 1H), 10.78 (s, 1H), 9.18 (s, 1H), 8.97 (s, 1H), 8.35 (s, 1H), 8.09 (t, J=7.7 Hz, 1H), 7.92 (s, 1H), 7.51 (d, J=7.4 Hz, 1H), 7.25 (d, J=32.4 Hz, 1H), 6.29-6.77 (q, J=30.4 Hz, 4H), 6.65 (d, J=7.9 Hz, 2H), 5.01 (s, 1H), 4.23 (d, J=7.8 Hz, 1H), 3.46 (s, 3H), 3.16 (s, 1H), 2.95 (s, 2H), 2.75 (m, 5H), 2.09 (m, 5H), 1.86 (m, 5H), 1.41-1.48 (m, 9H), 1.24-1.27 (m, 3H). LC-MS (ES⁺): m/z 757.18 [M+H]⁺.

### Example 21 Compound of Example 21 was Prepared Substantially Following the Synthesis of Example 15

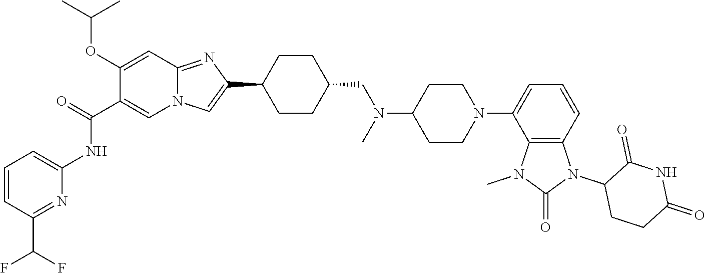 [SMILES: CC(C)Oc1cc2nc([C@H]3CC[C@H](CN(C)C4CCN(c5cccc6c5n(C)c(=O)n6C5CCC(=O)NC5=O)CC4)CC3)cn2cc1C(=O)Nc1cccc(C(F)F)n1]

### N-[6-(difluoromethyl)-2-pyridyl]-2-[4-[[[1-[1-(2,6-dioxo-3-piperidyl)-3-methyl-2-oxo-benzimidazol-4-yl]-4-piperidyl]-methyl-amino]methyl]cyclohexyl]-7-isopropoxy-imidazo[1,2-a]pyridine-6-carboxamide

¹H NMR (400 MHz, DMSO-d₆) δ 11.10 (s, 1H), 9.16 (s, 1H), 8.89 (s, 1H), 8.36 (s, 1H), 8.09 (t, J=7.9 Hz, 1H), 7.84 (s, 1H), 7.50 (d, J=7.1 Hz, 1H), 7.21 (s, 1H), 7.07-6.76 (m, 5H), 5.36 (t, J=6.2 Hz, 1H), 5.00 (s, 1H), 3.66 (s, 3H), 3.34 (m, 4H), 2.50-2.95 (m, 8H), 2.11-2.32 (m, 10H), 2.01 (m, 8H), 1.38 (m, 3H). LC-MS (ES⁺): m/z 812.12 [M+H]⁺.

### Example 22 Compound of Example 22 was Prepared Substantially Following the Synthesis of Example 15

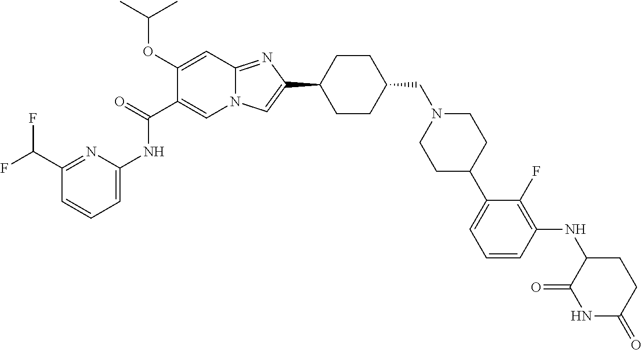 [SMILES: CC(C)Oc1cc2nc([C@H]3CC[C@H](CN4CCC(c5cccc(NC6CCC(=O)NC6=O)c5F)CC4)CC3)cn2cc1C(=O)Nc1cccc(C(F)F)n1]

### N-[6-(difluoromethyl)-2-pyridyl]-2-[4-[[4-[3-[(2,6-dioxo-3-piperidyl)amino]-2-fluoro-phenyl]-1-piperidyl]methyl]cyclohexyl]-7-isopropoxy-imidazo[1,2-a]pyridine-6-carboxamide

¹H NMR (400 MHz, DMSO-d₆) δ 10.85 (s, 1H), 10.78 (s, 1H), 9.10 (s, 1H), 8.37 (d, J=8.3 Hz, 1H), 8.07 (t, J=8.0 Hz, 1H), 7.66 (s, 1H), 7.47 (d, J=7.6 Hz, 1H), 6.93 (m, 1H), 6.43 (d, J=12.0 Hz, 1H), 5.99 (d, J=7.7 Hz, 1H), 4.96 (q, J=5.8 Hz, 1H), 4.32 (d, J=11.7 Hz, 1H), 2.92 (d, J=10.7 Hz, 2H), 2.70 (d, J=27.2 Hz, 1H), 2.59 (m, 4H), 2.11 (m, 4H), 1.89 (m, 8H), 1.64 (m, 5H), 1.45 (m, 8H), 1.02 (d, J=10.9 Hz, 2H). LC-MS (ES⁺): m/z 746.13 [M+H]⁺.

### Example 23 Compound of Example 23 was Prepared Substantially Following the Synthesis of Example 15

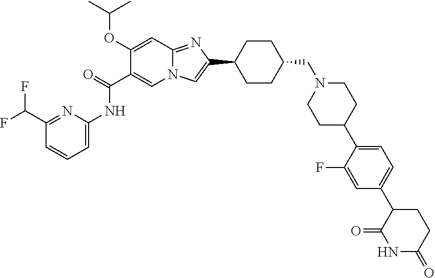 [SMILES: CC(C)Oc1cc2nc([C@H]3CC[C@H](CN4CCC(c5ccc(C6CCC(=O)NC6=O)cc5F)CC4)CC3)cn2cc1C(=O)Nc1cccc(C(F)F)n1]

### N-[6-(difluoromethyl)-2-pyridyl]-2-[4-[[4-[4-(2,6-dioxo-3-piperidyl)-2-fluoro-phenyl]-1-piperidyl]methyl]cyclohexyl]-7-isopropoxy-imidazo[1,2-a]pyridine-6-carboxamide

¹H NMR (400 MHz, DMSO-d₆) δ 10.84 (d, J=7.0 Hz, 1H), 9.10 (d, J=5.2 Hz, 1H), 8.33 (t, J=20.4 Hz, 1H), 8.30 (t, J=20.4 Hz, 3H), 8.07 (t, J=7.9 Hz, 1H), 7.70 (d, J=31.2 Hz, 1H), 7.47 (d, J=7.5 Hz, 1H), 7.30 (q, J=5.3 Hz, 1H), 7.07 (m, 2H), 6.82 (d, J=54.9 Hz, 1H), 4.95 (t, J=6.0 Hz, 1H), 3.86 (q, J=5.5 Hz, 1H), 2.96 (d, J=10.7 Hz, 3H), 2.64 (t, J=10.2 Hz, 1H), 2.50 (t, J=10.2 Hz, 1H), 2.18 (t, J=7.3 Hz, 3H), 1.99 (m, 6H), 1.73 (m, 6H), 1.45 (m, 8H), 1.03 (d, J=12.0 Hz, 1H), 0.31 (t, J=88.5 Hz, 1H). LC-MS (ES⁺): m/z 731.26 [M+H]⁺.

### Example 24 Compound of Example 24 was Prepared Substantially Following the Synthesis of Example 15

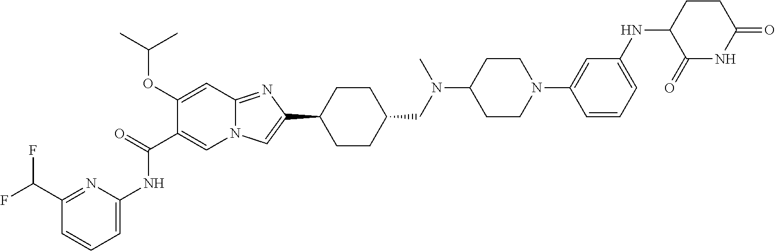 [SMILES: CC(C)Oc1cc2nc([C@H]3CC[C@H](CN(C)C4CCN(c5cccc(NC6CCC(=O)NC6=O)c5)CC4)CC3)cn2cc1C(=O)Nc1cccc(C(F)F)n1]

### N-[6-(difluoromethyl)-2-pyridyl]-2-[4-[[[1-[3-[(2,6-dioxo-3-piperidyl)amino]phenyl]-4-piperidyl]-methyl-amino]methyl]cyclohexyl]-7-isopropoxy-imidazo[1,2-a]pyridine-6-carboxamide

¹H NMR (400 MHz, DMSO-d₆) δ 10.85 (s, 1H), 10.75 (d, J=6.3 Hz, 1H), 9.09 (d, J=4.9 Hz, 1H), 8.37 (d, J=8.4 Hz, 1H), 8.23 (s, 1H), 8.07 (t, J=8.0 Hz, 1H), 7.72 (d, J=27.9 Hz, 1H), 7.65 (d, J=27.9 Hz, 1H), 7.47 (d, J=7.5 Hz, 1H), 7.12 (d, J=12.3 Hz, 1H), 6.89 (m, 2H), 6.59 (s, 1H), 6.10-6.26 (m, 3H), 5.60 (t, J=7.1 Hz, 1H), 4.95 (m, 1H), 4.32 (d, J=6.8 Hz, 1H), 3.66 (d, J=10.6 Hz, 2H), 2.76 (m, 1H), 2.22 (m, 1H), 2.42 (m, 4H), 1.90-2.08 (m, 5H), 1.86 (m, 3H), 1.73 (m, 3H), 1.0-1.48 (m, 12H), 0.99 (m, 1H). LC-MS (ES⁺): m/z 757.22 [M+H]⁺.

### Example 25 Compound of Example 25 was Prepared Substantially Following the Synthesis of Example 15

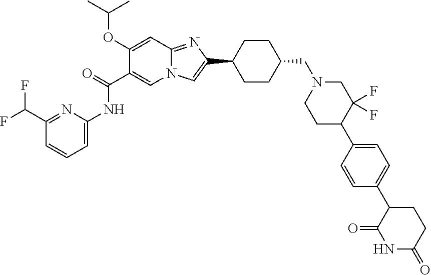 [SMILES: CC(C)Oc1cc2nc([C@H]3CC[C@H](CN4CCC(c5ccc(C6CCC(=O)NC6=O)cc5)C(F)(F)C4)CC3)cn2cc1C(=O)Nc1cccc(C(F)F)n1]

### N-[6-(difluoromethyl)-2-pyridyl]-2-[4-[[4-[4-(2,6-dioxo-3-piperidyl)phenyl]-3,3-difluoro-1-piperidyl]methyl]cyclohexyl]-7-isopropoxy-imidazo[1,2-a]pyridine-6-carboxamide

¹H NMR (400 MHz, DMSO-d₆) δ 10.86 (s, 1H), 9.10 (d, J=5.6 Hz, 1H), 8.37 (d, J=8.5 Hz, 1H), 8.07 (t, J=8.0 Hz, 1H), 7.70 (d, J=33.2 Hz, 1H), 7.47 (d, J=7.5 Hz, 1H), 7.25 (t, J=18.9 Hz, 2H), 7.18 (s, 2H), 7.13 (d, J=10.9 Hz, 1H), 6.89 (t, J=54.8 Hz, 1H), 4.95 (t, J=6.0 Hz, 1H), 3.84 (q, J=5.5 Hz, 1H), 3.31-2.62 (m, 4H), 2.59 (m, 2H), 2.20 (m, 7H), 1.85 (m, 4H), 1.59 (m, 1H), 1.49 (m, 8H), 1.39 (m, 1H), 1.28 (m, 1H). 1.11 (m, 2H). LC-MS (ES⁺): m/z 749.16 [M+H]⁺.

### Example 26 Compound of Example 26 was Prepared Substantially Following the Synthesis of Example 15

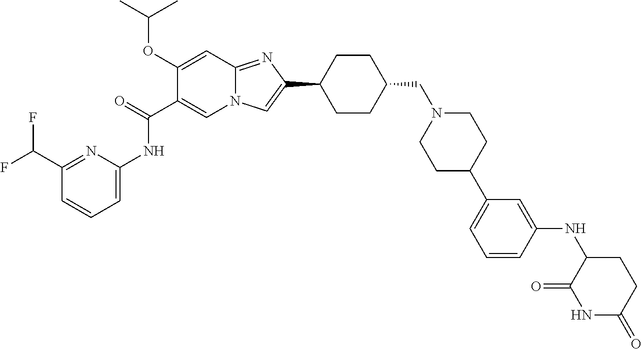 [SMILES: CC(C)Oc1cc2nc([C@H]3CC[C@H](CN4CCC(c5cccc(NC6CCC(=O)NC6=O)c5)CC4)CC3)cn2cc1C(=O)Nc1cccc(C(F)F)n1]

### N-[6-(difluoromethyl)-2-pyridyl]-2-[4-[[4-[3-[(2,6-dioxo-3-piperidyl)amino]phenyl]-1-piperidyl]methyl]cyclohexyl]-7-isopropoxy-imidazo[1,2-a]pyridine-6-carboxamide

¹H NMR (400 MHz, DMSO-d₆) δ 10.81 (d, J=36.7 Hz, 1H), 10.76 (d, J=36.7 Hz, 1H), 9.11 (s, 1H), 8.37 (d, J=8.3 Hz, 1H), 8.07 (t, J=7.9 Hz, 1H), 7.73 (s, 1H), 7.47 (d, J=7.6 Hz, 1H), 7.12 (d, J=11.9 Hz, 1H), 6.96 (m, 1H), 6.50 (m, 2H), 5.74 (d, 1H), 4.96 (q, J=5.9 Hz, 1H), 4.33 (s, 1H), 3.31-2.80 (m, 4H), 2.67 (m, 4H), 2.08 (m, 5H), 1.91 (t, J=5.8 Hz, 4H), 1.72 (m, 4H), 1.45 (m, 9H), 1.35(m, 1H) (1.09 (m, 2H). LC-MS (ES⁺): m/z 728.20 [M+H]⁺.

### Example 27 Compound of Example 27 was Prepared Substantially Following the Synthesis of Example 15

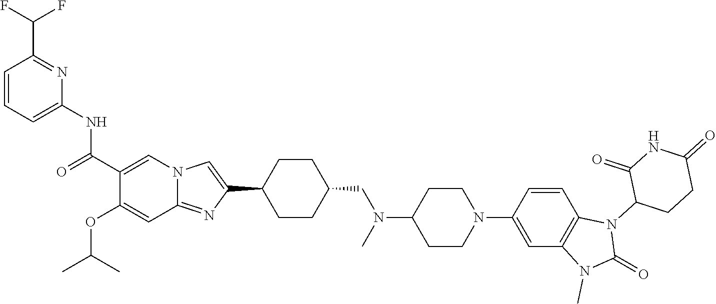 [SMILES: CC(C)Oc1cc2nc([C@H]3CC[C@H](CN(C)C4CCN(c5ccc6c(c5)n(C)c(=O)n6C5CCC(=O)NC5=O)CC4)CC3)cn2cc1C(=O)Nc1cccc(C(F)F)n1]

### N-[6-(difluoromethyl)-2-pyridyl]-2-[4-[[[1-[1-(2,6-dioxo-3-piperidyl)-3-methyl-2-oxo-benzimidazol-5-yl]-4-piperidyl]-methyl-amino]methyl]cyclohexyl]-7-isopropoxy-imidazo[1,2-a]pyridine-6-carboxamide

¹H NMR (400 MHz, DMSO-d₆) δ 11.06 (s, 1H), 10.85 (s, 1H), 9.10 (s, 1H), 8.37 (d, J=8.3 Hz, 1H), 8.27 (s, 1H), 8.07 (t, J=8.0 Hz, 1H), 7.66 (s, 1H), 7.47 (d, J=7.6 Hz, 1H), 7.07 (d, J=34.2 Hz, 1H), 6.84 (m, 3H), 6.64 (m, 2H), 6.28 (s, 1H), 5.28 (m, 1H), 4.95 (m, 1H), 3.64 (d, J=11.5 Hz, 2H), 3.30 (d, J=11.5 Hz, 3H), 2.60 (m, 1H), 2.50 (m, 7H), 2.24 (m, 5H), 1.99 (q, J=26.7 Hz, 2H), 1.77 (d, J=12.0 Hz, 2H), 1.59 (m, 2H), 1.44 (m, 8H), 1.00 (m, 2H). LC-MS (ES⁺): m/z 812.32 [M+H]⁺.

### Example 28 Compound of Example 28 was Prepared Substantially Following the Synthesis of Example 15

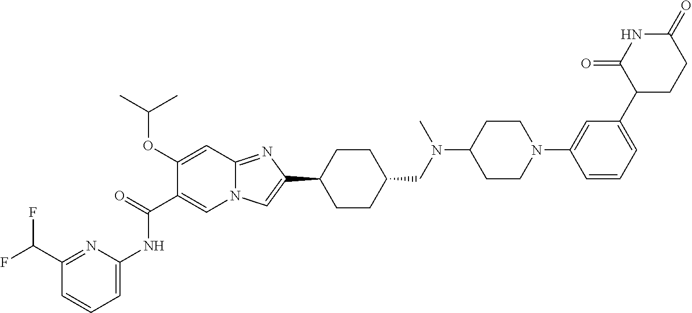 [SMILES: CC(C)Oc1cc2nc([C@H]3CC[C@H](CN(C)C4CCN(c5cccc(C6CCC(=O)NC6=O)c5)CC4)CC3)cn2cc1C(=O)Nc1cccc(C(F)F)n1]

### N-[6-(difluoromethyl)-2-pyridyl]-2-[4-[[[1-[3-(2,6-dioxo-3-piperidyl)phenyl]-4-piperidyl]-methyl-amino]methyl]cyclohexyl]-7-isopropoxy-imidazo[1,2-a]pyridine-6-carboxamide

¹H NMR (400 MHz, DMSO-d₆) δ 12.65 (s, 1H), 10.93 (s, 1H), 10.80 (d, J=5.1 Hz, 1H), 9.14 (s, 1H), 8.81 (s, 1H), 8.36 (d, J=7.8 Hz, 1H), 8.10 (q, J=9.4 Hz, 1H), 7.76 (s, 1H), 7.49 (d, J=7.5 Hz, 4H), 7.12 (m, J=11.7 Hz, 1H), 6.86 (m, J=13.0 Hz, 1H), 6.59 (q, J=17.9 Hz, 1H), 4.97 (s, 1H), 3.80 (m, J=9.0 Hz, 1H), 3.17 (d, J=5.3 Hz, 4H), 2.95 (t, J=10.5 Hz, 5H), 2.71 (m, J=8.2 Hz, 1H), 2.11 (m, 7H), 1.82 (m, 5H), 1.43 (m, 8H), 1.22 (m, 2H). LC-MS (ES⁺): m/z 742.18 [M+H]⁺.

### Example 29 Compound of Example 29 was Prepared Substantially Following the Synthesis of Example 15

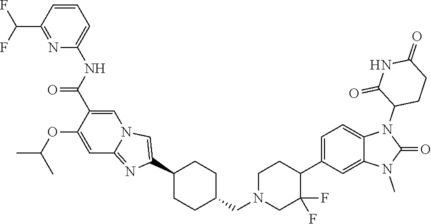 [SMILES: CC(C)Oc1cc2nc([C@H]3CC[C@H](CN4CCC(c5ccc6c(c5)n(C)c(=O)n6C5CCC(=O)NC5=O)C(F)(F)C4)CC3)cn2cc1C(=O)Nc1cccc(C(F)F)n1]

### N-[6-(difluoromethyl)-2-pyridyl]-2-[4-[[4-[1-(2,6-dioxo-3-piperidyl)-3-methyl-2-oxo-benzimidazol-5-yl]-3,3-difluoro-1-piperidyl]methyl]cyclohexyl]-7-isopropoxy-imidazo[1,2-a]pyridine-6-carboxamide

¹H NMR (400 MHz, DMSO-d₆) δ 11.10 (s, 1H), 10.86 (s, 1H), 9.10 (d, J=3.8 Hz, 1H), 8.37 (d, J=8.3 Hz, 1H), 8.28 (s, 1H), 8.07 (t, J=8.0 Hz, 1H), 7.70 (d, J=31.3 Hz, 1H), 7.47 (d, J=7.5 Hz, 1H), 7.07 (m, 2H), 6.82 (d, J=54.9 Hz, 2H), 6.61 (s, 3H), 6.28 (s, 1H), 5.36 (q, =6.0 Hz, 1H), 4.95 (m 1H), 2.90 (m, 6H), 2.66 (m, 1H), 2.29 (m, 1H), 2.05 (m, 7H), 1.77 (m, 2H), 1.61 (s, 1H), 1.45 (m, 8H), 1.06 (s, 2H). LC-MS (ES⁺): m/z 819.10 [M+H]⁺.

### Example 30 Compound of Example 30 was Prepared Substantially Following the Synthesis of Example 15

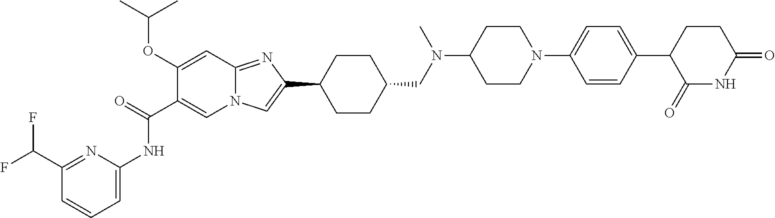 [SMILES: CC(C)Oc1cc2nc([C@H]3CC[C@H](CN(C)C4CCN(c5ccc(C6CCC(=O)NC6=O)cc5)CC4)CC3)cn2cc1C(=O)Nc1cccc(C(F)F)n1]

### N-[6-(difluoromethyl)-2-pyridyl]-2-[4-[[[1-[4-(2,6-dioxo-3-piperidyl)phenyl]-4-piperidyl]-methyl-amino]methyl]cyclohexyl]-7-isopropoxy-imidazo[1,2-a]pyridine-6-carboxamide

¹H NMR (400 MHz, DMSO-d₆) δ 10.81 (t, J=19.0 Hz, 1H), 9.10 (d, J=2.3 Hz, 1H), 8.37 (d, J=8.2 Hz, 1H), 8.07 (t, J=8.0 Hz, 1H), 7.70 (d, J=28.0 Hz, 1H), 7.47 (d, J=7.5 Hz, 1H), 7.13-6.75 (m, 6H), 6.5 (s, 1H), 4.96 (q, J=6.0 Hz, 1H), 3.74 (m, 3H), 2.65 (m, 1H), 2.59-2.32 (m, 4H), 2.06 (m, 9H), 1.89 (m, 4H), 1.60 (m, 8H), 1.44 (d, J=6.0 Hz, 2H), 1.05 (t, J=8.1 Hz, 1H), 0.53 (s, 1H), 0.31 (s, 1H), 0.09 (s, 1H). LC-MS (ES⁺): m/z 742.18 [M+H]⁺.

### Example 31 Compound of Example 31 was Prepared Substantially Following the Synthesis of Example 15

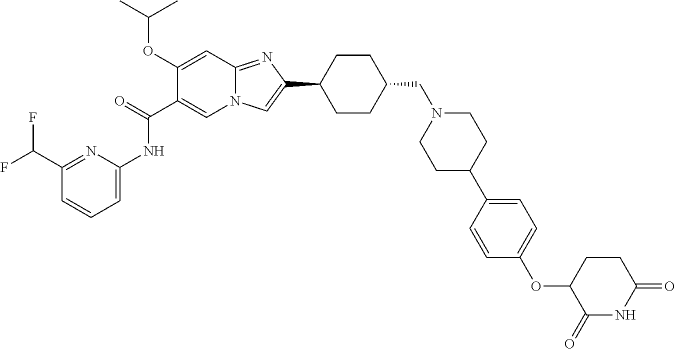 [SMILES: CC(C)Oc1cc2nc([C@H]3CC[C@H](CN4CCC(c5ccc(OC6CCC(=O)NC6=O)cc5)CC4)CC3)cn2cc1C(=O)Nc1cccc(C(F)F)n1]

### N-[6-(difluoromethyl)-2-pyridyl]-2-[4-[[4-[4-[(2,6-dioxo-3-piperidyl)oxy]phenyl]-1-piperidyl]methyl]cyclohexyl]-7-isopropoxy-imidazo[1,2-a]pyridine-6-carboxamide

¹H NMR (400 MHz, DMSO-d₆) δ 10.91 (s, 1H), 10.85 (s, 1H), 9.10 (s, 1H), 8.37 (d, J=8.3 Hz, 1H), 8.16 (s, 1H), 8.07 (t, J=8.0 Hz, 1H), 7.66 (s, 1H), 7.47 (d, J=7.5 Hz, 1H), 7.10 (q, J=19.4 Hz, 3H), 6.85 (q, J=25.8 Hz, 3H), 6.54 (s, 1H), 5.14 (q, J=5.3 Hz, 1H), 4.95 (m, 1H), 2.96 (d, J=10.9 Hz, 2H), 2.63 (m, 1H), 2.17 (q, J=7.4 Hz, 2H), 2.04 (m, 8H), 1.91 (d, J=11.8 Hz, 2H), 1.66 (m, 4H), 1.45 (m, 8H), 1.03 (q, J=11.8 Hz, 2H). LC-MS (ES⁺): m/z 729.16 [M+H]⁺.

### Example 32 Compound of Example 32 was Prepared Substantially Following the Synthesis of Example 15

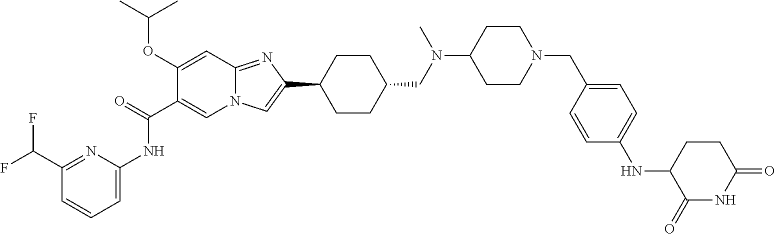 [SMILES: CC(C)Oc1cc2nc([C@H]3CC[C@H](CN(C)C4CCN(Cc5ccc(NC6CCC(=O)NC6=O)cc5)CC4)CC3)cn2cc1C(=O)Nc1cccc(C(F)F)n1]

### N-[6-(difluoromethyl)-2-pyridyl]-2-[4-[[[1-[[4-[(2,6-dioxo-3-piperidyl)amino]phenyl]methyl]-4-piperidyl]-methyl-amino]methyl]cyclohexyl]-7-isopropoxy-imidazo[1,2-a]pyridine-6-carboxamide

¹H NMR (400 MHz, DMSO-d₆) δ 10.86 (s, 1H), 10.78 (s, 1H), 9.10 (s, 1H), 8.42 (m, 2H), 8.08 (t, J=8.2 Hz, 1H), 7.65 (s, 1H), 7.47 (d, J=7.8 Hz, 1H), 7.11 (s, 1H), 6.98 (d, J=8.4 Hz, 1H), 6.89-6.60 (m, 8H), 5.75 (d, J=8.4 Hz, 1H), 4.91 (s, 1H), 4.28 (s, 1H), 2.82 (s, 2H), 2.60 (m, 2H), 2.08 (m, 7H), 1.81 (m, 5H), 1.41 (m, 8H), 1.20 (m, 7H). LC-MS (ES⁺): m/z 771.05 [M+H]⁺.

### Example 33 Compound of Example 33 was Prepared Substantially Following the Synthesis of Example 15

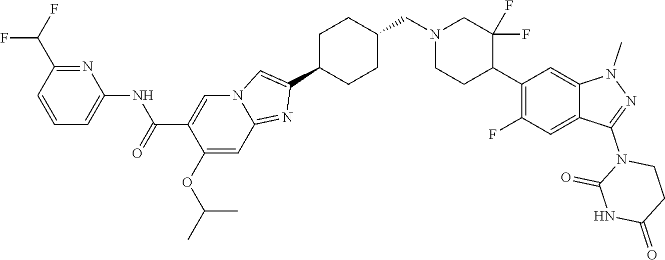 [SMILES: CC(C)Oc1cc2nc([C@H]3CC[C@H](CN4CCC(c5cc6c(cc5F)c(N5CCC(=O)NC5=O)nn6C)C(F)(F)C4)CC3)cn2cc1C(=O)Nc1cccc(C(F)F)n1]

### N-[6-(difluoromethyl)-2-pyridyl]-2-[4-[[4-[3-(2,4-dioxohexahydropyrimidin-1-yl)-5-fluoro-1-methyl-indazol-6-yl]-3,3-difluoro-1-piperidyl]methyl]cyclohexyl]-7-isopropoxy-imidazo[1,2-a]pyridine-6-carboxamide

¹H NMR (400 MHz, DMSO-d₆) δ 11.09 (s, 1H), 10.57 (s, 1H), 9.17 (d, J=6.9 Hz, 1H), 8.35 (s, 1H), 8.09 (t, J=7.8 Hz, 1H), 7.97 (d, J=33.1 Hz, 1H), 7.71 (s, 1H), 7.47 (m, 2H), 7.25 (d, J=27.1 Hz, 1H), 7.00 (t, J=36.9 Hz, 1H), 6.77 (s, 1H), 5.01 (s, 1H), 4.03 (d, J=1.2 Hz, 3H), 3.91 (d, J=6.4 Hz, 2H), 3.07 (m, 4H), 2.77 (d, J=6.6 Hz, 3H), 2.11 (t, J=13.0 Hz, 1H), 1.92 (m, 6H), 1.71 (m, 3H), 1.45-1.22 (m, 8H), 1.15 (s, 1H). LC-MS (ES⁺): m/z 822.08 [M+H]⁺.

### Example 34 Compound of Example 34 was Prepared Substantially Following the Synthesis of Example 15

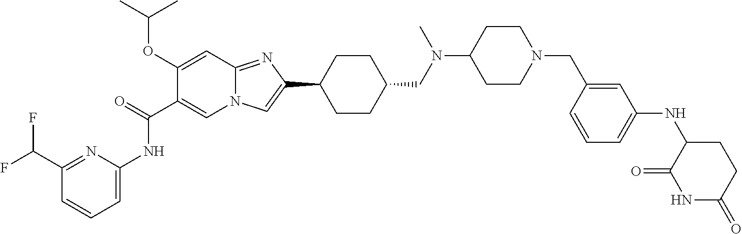 [SMILES: CC(C)Oc1cc2nc([C@H]3CC[C@H](CN(C)C4CCN(Cc5cccc(NC6CCC(=O)NC6=O)c5)CC4)CC3)cn2cc1C(=O)Nc1cccc(C(F)F)n1]

### N-[6-(difluoromethyl)-2-pyridyl]-2-[4-[[[1-[[3-[(2,6-dioxo-3-piperidyl)amino]phenyl]methyl]-4-piperidyl]-methyl-amino]methyl]cyclohexyl]-7-isopropoxy-imidazo[1,2-a]pyridine-6-carboxamide

¹H NMR (400 MHz, DMSO-d₆) δ 10.81 (t, J=18.1 Hz, 1H), 9.10 (d, J=2.7 Hz, 1H), 8.37 (d, J=8.3 Hz, 1H), 8.15-8.05 (m, 3H), 7.69 (d, J=27.2 Hz, 1H), 7.47 (d, J=7.4 Hz, 1H), 7.07 (m, 2H), 6.82 (d, J=54.9 Hz, 1H), 6.56 (m, 5H), 5.83 (d, J=7.4 Hz, 1H), 4.95 (t, J=6.0 Hz, 1H), 4.30 (d, J=6.7 Hz, 1H), 3.5 (s, 1H), 3.0 (s, 3H), 2.92 (s, 1H), 2.70 (m, 3H), 2.33 (d, J=1.7 Hz, 1H), 1.99 (m, 13H), 1.73 (m, 9H), 1.44 (d, J=6.0 Hz, 1H), 1.02 (d, J=12.8 Hz, 1H). LC-MS (ES⁺): m/z 771.17 [M+H]⁺.

### Example 35 Compound of Example 35 was Prepared Substantially Following the Synthesis of Example 15

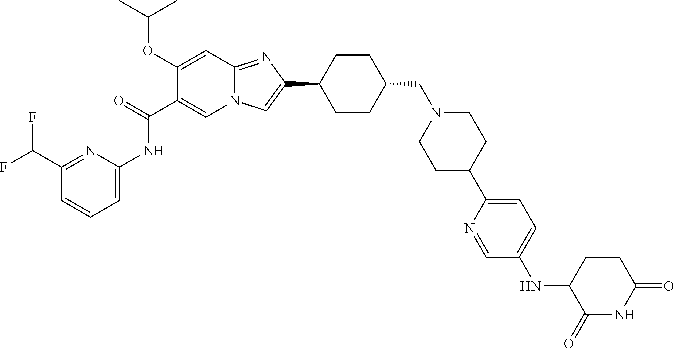 [SMILES: CC(C)Oc1cc2nc([C@H]3CC[C@H](CN4CCC(c5ccc(NC6CCC(=O)NC6=O)cn5)CC4)CC3)cn2cc1C(=O)Nc1cccc(C(F)F)n1]

### N-[6-(difluoromethyl)-2-pyridyl]-2-[4-[[4-[5-[(2,6-dioxo-3-piperidyl)amino]-2-pyridyl]-1-piperidyl]methyl]cyclohexyl]-7-isopropoxy-imidazo[1,2-a]pyridine-6-carboxamide

¹H NMR (400 MHz, DMSO-d₆) δ 10.82 (d, J=24.9 Hz, 1H), 9.10 (d, J=4.2 Hz, 1H), 8.37 (d, J=8.2 Hz, 1H), 8.17 (s, 1H), 8.07 (t, J=7.9 Hz, 1H), 7.97 (s, 1H), 7.70 (d, J=30.7 Hz, 1H), 7.47 (d, J=7.5 Hz, 1H), 7.13 (d, J=11.7 Hz, 1H), 6.95 (t, J=27.5 Hz, 3H), 6.64 (d, J=85.3 Hz, 3H), 5.92 (d, J=7.7 Hz, 1H), 4.95 (t, J=5.9 Hz, 1H), 4.34 (s, 1H), 3.32 (m, 3H), 2.97 (s, 1H), 2.67 (m, 2H), 2.50 (s, 2H), 2.24 (s, 1H), 2.07 (m, 3H), 1.90 (m, 3H), 1.67 (m, 6H), 1.45 (m, 6H), 1.05 (s, 1H). LC-MS (ES⁺): m/z 760.16 [M+H]⁺.

### Example 36 Compound of Example 36 was Prepared Substantially Following the Synthesis of Example 15

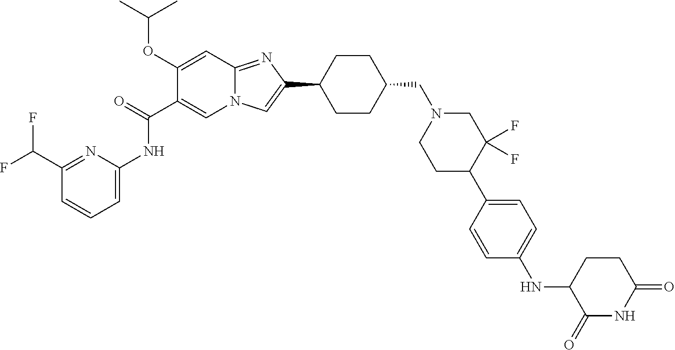 [SMILES: CC(C)Oc1cc2nc([C@H]3CC[C@H](CN4CCC(c5ccc(NC6CCC(=O)NC6=O)cc5)C(F)(F)C4)CC3)cn2cc1C(=O)Nc1cccc(C(F)F)n1]

### N-[6-(difluoromethyl)-2-pyridyl]-2-[4-[[4-[4-[(2,6-dioxo-3-piperidyl)amino]phenyl]-3,3-difluoro-1-piperidyl]methyl]cyclohexyl]-7-isopropoxy-imidazo[1,2-a]pyridine-6-carboxamide

¹H NMR (400 MHz, DMSO-d₆) δ 10.82 (d, J=29.9 Hz, 1H), 9.11 (s, 1H), 8.37 (d, J=8.5 Hz, 1H), 8.07 (t, J=7.9 Hz, 1H), 7.66 (s, 1H), 7.47 (d, J=7.5 Hz, 1H), 7.07 (d, J=36.0 Hz, 3H), 6.88 (t, J=51.0 Hz, 1H), 6.63 (d, J=8.5 Hz, 2H), 5.80 (d, J=7.5 Hz, 1H), 4.95 (m, 1H), 4.30 (m, 1H), 3.03 (q, J=27.0 Hz, 1H), 2.71 (t, J=15.3 Hz, 2H), 2.59 (t, J=1.7 Hz, 4H), 2.50 (t, J=1.7 Hz, 2H), 2.25 (t, J=10.4 Hz, 3H), 2.11 (q, J=9.2 Hz, 4H), 1.90 (t, J=12.2 Hz, 3H), 1.74 (d, J=8.9 Hz, 1H), 1.46 (m, 1H), 1.41-1.45 (m, 8H), 1.04 (d, J=12.1 Hz, 1H). LC-MS (ES⁺): m/z 764. 20 [M+H]⁺.

### Example 37 Compound of Example 37 was Prepared Substantially Following the Synthesis of Example 15

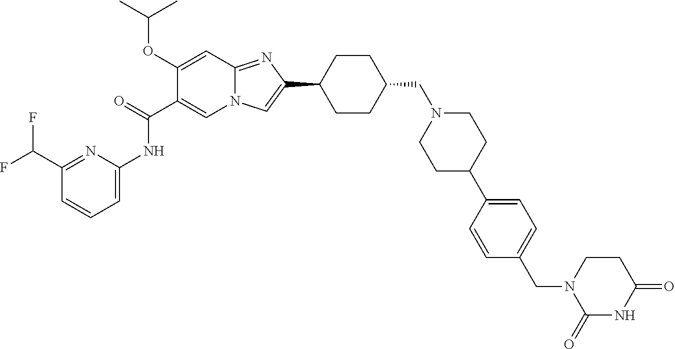 [SMILES: CC(C)Oc1cc2nc([C@H]3CC[C@H](CN4CCC(c5ccc(CN6CCC(=O)NC6=O)cc5)CC4)CC3)cn2cc1C(=O)Nc1cccc(C(F)F)n1]

### N-[6-(difluoromethyl)-2-pyridyl]-2-[4-[[4-[4-[(2,4-dioxohexahydropyrimidin-1-yl)methyl]phenyl]-1-piperidyl]methyl]cyclohexyl]-7-isopropoxy-imidazo[1,2-a]pyridine-6-carboxamide

¹H NMR (400 MHz, DMSO-d₆) δ 10.85 (s, 1H), 10.18 (s, 1H), 9.10 (d, J=5.0 Hz, 1H), 8.37 (d, J=8.3 Hz, 1H), 8.11 (q, J=15.6 Hz, 1H), 8.0 (q, J=15.6 Hz, 1H), 7.70 (d, J=30.9 Hz, 1H), 7.47 (d, J=7.5 Hz, 3H), 7.17 (t, J=21.9 Hz, 1H), 6.89 (t, J=54.9 Hz, 1H), 6.55 (s, 1H), 4.95 (m, 1H), 4.47 (s, 2H), 3.32 (d, J=10.3 Hz, 2H), 2.96 (m, 3H), 2.50 (m, 5H), 2.19 (t, J=7.9 Hz, 2H), 1.99 (m, 5H), 1.67 (m, 6H), 1.45 (m, 7H), 1.03 (q, J=11.6 Hz, 1H). LC-MS (ES⁻): m/z 726.39 [M−H]⁻

### Example 38 Compound of Example 38 was Prepared Substantially Following the Synthesis of Example 15

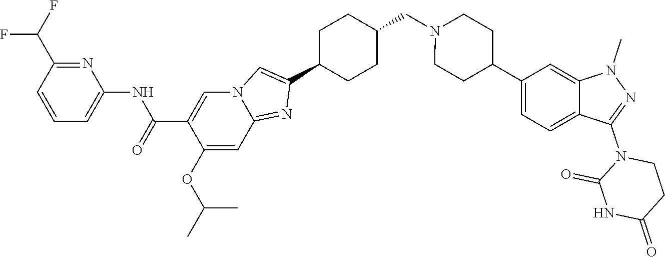 [SMILES: CC(C)Oc1cc2nc([C@H]3CC[C@H](CN4CCC(c5ccc6c(N7CCC(=O)NC7=O)nn(C)c6c5)CC4)CC3)cn2cc1C(=O)Nc1cccc(C(F)F)n1]

### N-[6-(difluoromethyl)-2-pyridyl]-2-[4-[[4-[3-(2,4-dioxohexahydropyrimidin-1-yl)-1-methyl-indazol-6-yl]-1-piperidyl]methyl]cyclohexyl]-7-isopropoxy-imidazo[1,2-a]pyridine-6-carboxamide

¹H NMR (400 MHz, DMSO-d₆) δ 11.07 (s, 1H), 10.56 (s, 1H), 9.19 (d, J=6.0 Hz, 1H), 8.89 (d, J=3.0 Hz, 1H), 8.34 (t, J=21.8 Hz, 1H), 8.10 (t, J=8.1 Hz, 1H), 7.94 (d, J=17.5 Hz, 1H), 7.64 (d, J=8.6 Hz, 1H), 7.52 (t, J=7.1 Hz, 1H), 7.41 (s, 1H), 7.24 (d, J=29.1 Hz, 1H), 6.99 (m, 1H), 5.02 (s, 1H), 3.99 (d, J=1.3 Hz, 3H), 3.92 (t, J=6.6 Hz, 2H), 3.65 (s, 2H), 3.13 (m, 6H), 2.72 (m, 3H), 2.04-1.53 (m, 9H), 1.43 (m, 7H), 1.20 (m, 2H). LC-MS (ES⁺): m/z 766.47 [M+H]⁺.

### Example 39 Compound of Example 39 was Prepared Substantially Following the Synthesis of Example 15

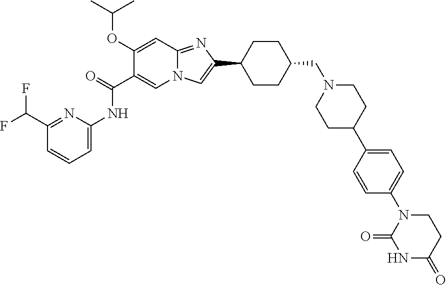 [SMILES: CC(C)Oc1cc2nc([C@H]3CC[C@H](CN4CCC(c5ccc(N6CCC(=O)NC6=O)cc5)CC4)CC3)cn2cc1C(=O)Nc1cccc(C(F)F)n1]

### N-[6-(difluoromethyl)-2-pyridyl]-2-[4-[[4-[4-(2,4-dioxohexahydropyrimidin-1-yl)phenyl]-1-piperidyl]methyl]cyclohexyl]-7-isopropoxy-imidazo[1,2-a]pyridine-6-carboxamide

¹H NMR (401 MHz, DMSO-d₆) δ 11.06 (s, 1H), 10.37 (s, 1H), 9.18 (s, 1H), 8.94 (s, 1H), 8.35 (s, 1H), 8.09 (t, J=7.9 Hz, 1H), 7.94 (d, J=18.0 Hz, 1H), 7.45 (t, J=26.7 Hz, 1H), 7.29 (q, J=8.1 Hz, 1H), 6.91 (m, 5H), 5.01 (s, 1H), 3.77 (t, J=6.6 Hz, 2H), 3.20 (m, 3H), 2.84 (d, J=11.2 Hz, 2H), 2.69 (q, J=6.7 Hz, 2H), 2.03 (m, 10H), 1.17 (t, J=23.2 Hz, 1H), 1.47 (t, J=23.2 Hz, 8H), 1.21 (d, J=11.4 Hz, 2H). LC-MS (ES⁺): m/z 714.31 [M+H]⁺.

### Example 40 Compound of Example 40 was Prepared Substantially Following the Synthesis of Example 15

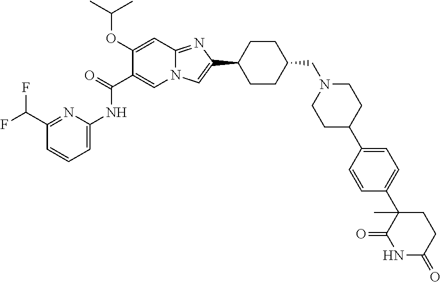 [SMILES: CC(C)Oc1cc2nc([C@H]3CC[C@H](CN4CCC(c5ccc(C6(C)CCC(=O)NC6=O)cc5)CC4)CC3)cn2cc1C(=O)Nc1cccc(C(F)F)n1]

### N-[6-(difluoromethyl)-2-pyridyl]-7-isopropoxy-2-[4-[[4-[4-(3-methyl-2,6-dioxo-3-piperidyl)phenyl]-1-piperidyl]methyl]cyclohexyl]imidazo[1,2-a]pyridine-6-carboxamide

¹H NMR (400 MHz, DMSO-d₆) δ 10.87 (d, J=14.9 Hz, 1H), 9.10 (d, J=5.5 Hz, 1H), 8.37 (d, J=8.3 Hz, 1H), 8.25 (s, 1H), 8.07 (t, J=7.9 Hz, 1H), 7.69 (d, J=31.3 Hz, 1H), 7.47 (d, J=7.5 Hz, 1H), 7.23 (m, 4H), 6.94 (m, 1H), 4.95 (t, J=5.9 Hz, 1H), 3.33 (d, J=10.3 Hz, 3H), 2.94 (d, J=10.3 Hz, 2H), 2.40 (t, J=27.1 Hz, 3H), 2.11 (m, 5H), 1.94 (m, 4H), 1.67 (m, 7H), 1.44 (m, 9H), 1.04 (s, 1H). LC-MS (ES⁺): m/z 727.22 [M+H]⁺.

### Example 41 Compound of Example 41 was Prepared Substantially Following the Synthesis of Example 15

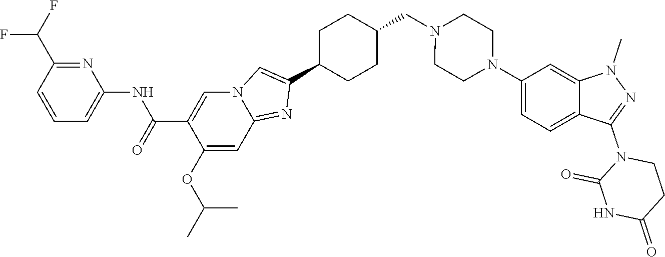 [SMILES: CC(C)Oc1cc2nc([C@H]3CC[C@H](CN4CCN(c5ccc6c(N7CCC(=O)NC7=O)nn(C)c6c5)CC4)CC3)cn2cc1C(=O)Nc1cccc(C(F)F)n1]

### N-[6-(difluoromethyl)-2-pyridyl]-2-[4-[[4-[3-(2,4-dioxohexahydropyrimidin-1-yl)-1-methyl-indazol-6-yl]piperazin-1-yl]methyl]cyclohexyl]-7-isopropoxy-imidazo[1,2-a]pyridine-6-carboxamide

¹H NMR (400 MHz, DMSO-d₆) δ 11.07 (s, 1H), 10.53 (s, 1H), 9.37 (s, 1H), 9.27 (t, J=35.9 Hz, 1H), 8.23 (d, J=92.9 Hz, 1H), 8.09 (t, J=7.9 Hz, 1H), 7.95 (d, J=14.3 Hz, 1H), 7.52 (t, J=8.9 Hz, 2H), 7.31-6.98 (m, 3H), 5.02 (s, 1H), 3.91 (q, J=4.9 Hz, 8H), 3.16 (m, 7H), 2.75 (t, J=6.6 Hz, 2H), 2.33 (m, 1H), 2.07-1.90 (m, 3H), 1.85 (m, 1H), 1.48 (m 8H), 1.22 (m, 2H). LC-MS (ES⁺): m/z 769.22 [M+H]⁺.

### Example 42 Compound of Example 42 was Prepared Substantially Following the Synthesis of Example 15

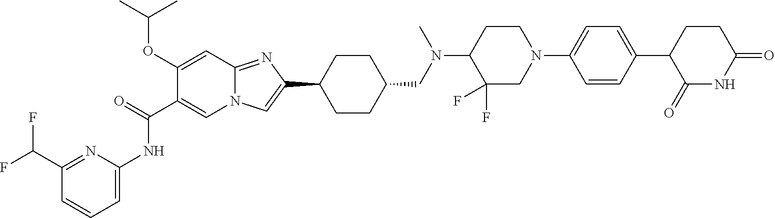 [SMILES: CC(C)Oc1cc2nc([C@H]3CC[C@H](CN(C)C4CCN(c5ccc(C6CCC(=O)NC6=O)cc5)CC4(F)F)CC3)cn2cc1C(=O)Nc1cccc(C(F)F)n1]

### N-[6-(difluoromethyl)-2-pyridyl]-2-[4-[[[1-[4-(2,6-dioxo-3-piperidyl)phenyl]-3,3-difluoro-4-piperidyl]-methyl-amino]methyl]cyclohexyl]-7-isopropoxy-imidazo[1,2-a]pyridine-6-carboxamide

¹H NMR (400 MHz, DMSO-d₆) δ 10.85 (s, 1H), 10.78 (s, 1H), 9.10 (s, 1H), 8.45 (s, 1H), 8.37 (d, J=8.3 Hz, 3H), 8.07 (t, J=7.9 Hz, 1H), 7.66 (s, 1H), 7.47 (d, J=7.5 Hz, 1H), 7.11 (s, 1H), 7.04-6.74 (m, 4H), 4.95 (t, J=6.0 Hz, 1H), 3.77 (m, 3H), 3.34 (m, 2H), 2.83 (m, 1H), 2.62 (m, 3H), 2.58 (m, 3H), 2.00 (m, 8H), 1.44 (m, 9H), 0.97 (t, J=12.2 Hz, 2H). LC-MS (ES⁺): m/z 778.41 [M+H]⁺.

### Example 43 Compound of Example 43 was Prepared Substantially Following the Synthesis of Example 15

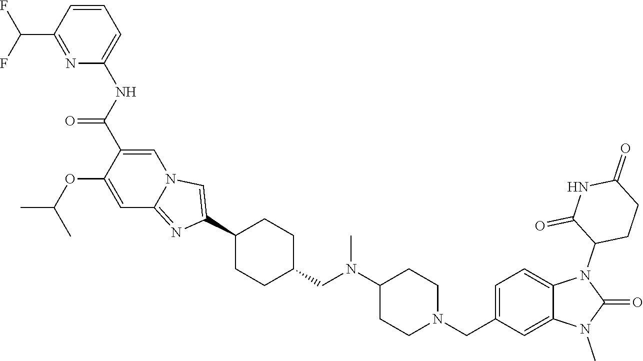 [SMILES: CC(C)Oc1cc2nc([C@H]3CC[C@H](CN(C)C4CCN(Cc5ccc6c(c5)n(C)c(=O)n6C5CCC(=O)NC5=O)CC4)CC3)cn2cc1C(=O)Nc1cccc(C(F)F)n1]

### N-[6-(difluoromethyl)-2-pyridyl]-2-[4-[[[1-[[1-(2,6-dioxo-3-piperidyl)-3-methyl-2-oxo-benzimidazol-5-yl]methyl]-4-piperidyl]-methyl-amino]methyl]cyclohexyl]-7-isopropoxy-imidazo[1,2-a]pyridine-6-carboxamide

¹H NMR (400 MHz, DMSO-d₆) δ 11.10 (s, 1H), 10.85 (s, 1H), 9.11 (s, 1H), 8.37 (d, J=8.2 Hz, 1H), 8.10 (d, J=24.7 Hz, 1H), 8.05 (d, J=24.7 Hz, 1H), 7.69 (d, J=26.7 Hz, 1H), 7.47 (d, J=7.5 Hz, 1H), 7.07 (m, 2H), 6.82 (d, J=54.9 Hz, 1H), 6.52 (s, 1H), 5.37 (q, J=6.0 Hz, 1H), 4.95 (t, J=6.1 Hz, 1H), 3.52 (d, J=4.4 Hz, 2H),), 3.31 (m, 3H), 3.02 (m, 3H), 2.65 (m, 4H), 2.50 (m, 5H), 1.95 (m, 14H), 1.60 (m, 4H), 1.39 (m, 3H), 1.06 (d, J=9.8 Hz, 1H). LC-MS (ES⁺): m/z 826.30 [M+H]⁺.

### Example 44 Compound of Example 44 was Prepared Substantially Following the Synthesis of Example 15

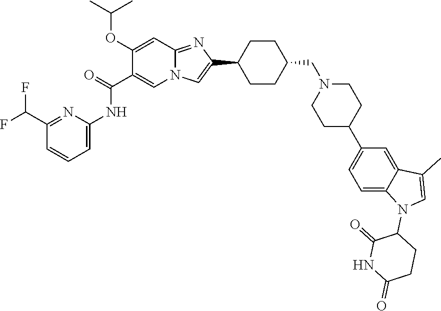 [SMILES: Cc1cn(C2CCC(=O)NC2=O)c2ccc(C3CCN(C[C@H]4CC[C@H](c5cn6cc(C(=O)Nc7cccc(C(F)F)n7)c(OC(C)C)cc6n5)CC4)CC3)cc12]

### N-[6-(difluoromethyl)-2-pyridyl]-2-[4-[[4-[1-(2,6-dioxo-3-piperidyl)-3-methyl-indol-5-yl]-1-piperidyl]methyl]cyclohexyl]-7-isopropoxy-imidazo[1,2-a]pyridine-6-carboxamide

¹H NMR (400 MHz, DMSO-d₆) δ 10.86 (s, 1H), 9.11 (s, 1H), 8.37 (d, J=8.4 Hz, 1H), 8.14 (s, 1H), 8.07 (t, J=8.0 Hz, 1H), 7.71 (d, J=30.8 Hz, 1H), 7.47 (d, J=7.5 Hz, 1H), 7.31 (t, J=8.4 Hz, 1H), 7.08 (m, J=11.9 Hz, 3H), 6.82 (d, J=54.9 Hz, 1H), 6.52 (s, 1H), 5.50 (q, J=5.9 Hz, 1H), 4.96 (q, J=6.0 Hz, 1H), 3.13 (d, J=26.2 Hz, 2H), 2.87 (m, 2H), 2.64 (m, 6H), 2.36-2.22 (m, 3H), 2.09-1-81 (m, 11H), 1.79 (m, 8H), 1.45-1.23 (m, 1H), 1.15 (m, 1H). LC-MS (ES⁺): m/z 766.17 [M+H]⁺.

### Example 45 Compound of Example 45 was Prepared Substantially Following the Synthesis of Example 15

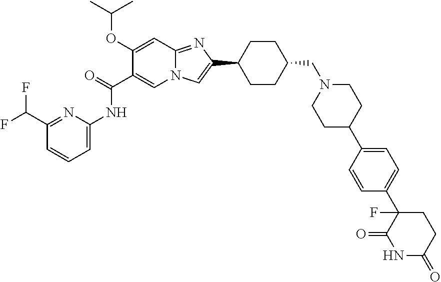 [SMILES: CC(C)Oc1cc2nc([C@H]3CC[C@H](CN4CCC(c5ccc(C6(F)CCC(=O)NC6=O)cc5)CC4)CC3)cn2cc1C(=O)Nc1cccc(C(F)F)n1]

### N-[6-(difluoromethyl)-2-pyridyl]-2-[4-[[4-[4-(3-fluoro-2,6-dioxo-3-piperidyl)phenyl]-1-piperidyl]methyl]cyclohexyl]-7-isopropoxy-imidazo[1,2-a]pyridine-6-carboxamide

¹H NMR (400 MHz, DMSO-d₆) δ 11.37 (s, 1H), 10.85 (s, 1H), 9.10 (s, 1H), 8.37 (d, J=10.8 Hz, 6H), 8.08 (d, J=7.9 Hz, 1H), 7.66 (s, 1H), 7.47 (d, J=7.5 Hz, 1H), 7.36 (s, 3H), 7.20 (d, J=3.9 Hz, 1H), 7.11 (s, 1H), 6.89 (t, J=54.9 Hz, 1H), 6.60 (s, 3H), 4.95 (s, 1H), 2.50 (m, 5H), 2.95 (d, J=10.6 Hz, 3H), 2.12 (m, 1H), 1.93 (m, 6H), 1.69 (m, 6H), 1.45 (d, J=6.0 Hz, 1H), 1.04 (s, 1H). LC-MS (ES⁺): m/z 731.21 [M+H]⁺.

### Example 46 Compound of Example 46 was Prepared Substantially Following the Synthesis of Example 15

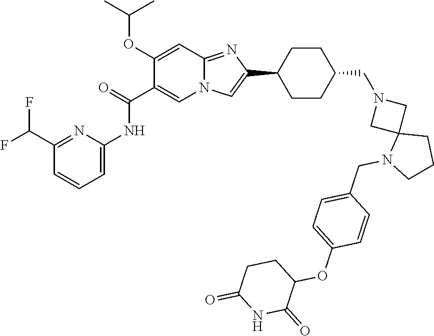 [SMILES: CC(C)Oc1cc2nc([C@H]3CC[C@H](CN4CC5(CCCN5Cc5ccc(OC6CCC(=O)NC6=O)cc5)C4)CC3)cn2cc1C(=O)Nc1cccc(C(F)F)n1]

### N-[6-(difluoromethyl)-2-pyridyl]-2-[4-[[5-[[4-[(2,6-dioxo-3-piperidyl)oxy]phenyl]methyl]-2,5-diazaspiro[3.4]octan-2-yl]methyl]cyclohexyl]-7-isopropoxy-imidazo[1,2-a]pyridine-6-carboxamide

¹H NMR (400 MHz, DMSO-d₆) δ 10.88 (d, J=25.5 Hz, 1H), 9.09 (d, J=5.4 Hz, 1H), 8.35 (t, J=12.9 Hz, 1H) 8.31 (s, 1H), 8.07 (t, J=8.0 Hz, 1H), 7.68 (d, J=25.7 Hz, 1H), 7.47 (d, J=7.5 Hz, 1H), 7.23 (d, 2H), 7.12 (d, J=8.4 Hz, 1H), 6.89 (m, 3H), 5.16 (q, J=5.2 Hz, 1H), 4.95 (m, 1H), 3.76 (d, J=7.7 Hz, 2H), 3.04 (d, 2H), 2.66 (m, 2H), 2.50 (d, 1H), 2.29 (m, 2H), 2.03 (m, 7H), 1.86 (d, 2H) 1.66 (t, 4H) 1.44 (m, 10H), 1.06 (m, 2H). LC-MS (ES⁺): m/z 770.32 [M+H]⁺.

### Example 47

### Synthesis of 2-[4-[[4-[4-(2,6-dioxo-3-piperidyl)-2-fluoro-phenyl]-1-piperidyl]methyl]cyclohexyl]-7-isopropoxy-N-pyrazolo[1,5-a]pyrimidin-3-yl-imidazo[1,2-a]pyridine-6-carboxamide

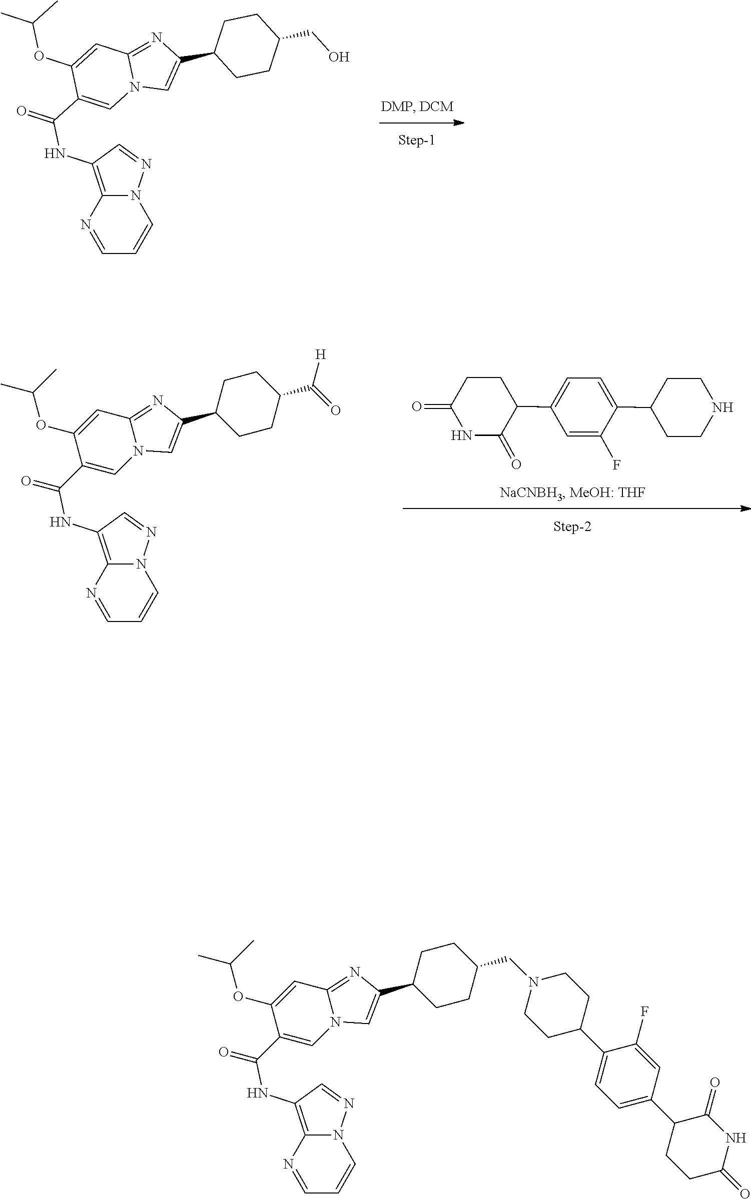 [SMILES: CC(C)Oc1cc2nc([C@H]3CC[C@H](CO)CC3)cn2cc1C(=O)Nc1cnn2cccnc12 ; CC(C)Oc1cc2nc([C@H]3CC[C@H](C=O)CC3)cn2cc1C(=O)Nc1cnn2cccnc12 ; O=C1CCC(c2ccc(C3CCNCC3)c(F)c2)C(=O)N1 ; CC(C)Oc1cc2nc([C@H]3CC[C@H](CN4CCC(c5ccc(C6CCC(=O)NC6=O)cc5F)CC4)CC3)cn2cc1C(=O)Nc1cnn2cccnc12]

### Step-1:

To a stirred solution of 2-[4-(hydroxymethyl)cyclohexyl]-7-isopropoxy-N-pyrazolo[1,5-a]pyrimidin-3-yl-imidazo[1,2-a]pyridine-6-carboxamide (0.05 g, 111.48 μmol) in chloroform (5 mL) was added Dess-Martin Periodinane (141.85 mg, 334.44 μmol) at 0-5° C. The reaction mixture was warmed up to 25° C. stirred for 4 hours. The progress of the reaction was monitored by LCMS/TLC. After completion of the reaction, the reaction was quenched with cold saturated sodium bicarbonate solution and extracted with DCM. The organic layer was washed with brine, dried over anhydrous Na₂SO₄ and concentrated in vacuo to afford the product 2-(4-formylcyclohexyl)-7-isopropoxy-N-pyrazolo[1,5-a]pyrimidin-3-yl-imidazo[1,2-a]pyridine-6-carboxamide (0.03 g, 50.39 μmol, 45.20% yield) as a yellow solid. LC-MS (ES⁺): m/z 447.47 [M+H]⁺.

### Step-2:

In a sealed tube, a solution of 2-(4-formylcyclohexyl)-7-isopropoxy-N-pyrazolo[1,5-a]pyrimidin-3-yl-imidazo[1,2-a]pyridine-6-carboxamide (0.06 g, 134.38 μmol), 3-[3-fluoro-4-(4-piperidyl)phenyl]piperidine-2,6-dione TFA salt (70.64 mg, 174.69 μmol) and TEA (13.60 mg, 134.38 μmol, 18.73 μL) in THE (2 mL) was stirred at 65° C. for 3 hours. The reaction mixture was warmed up to room temperature and sodium cyanoborohydride (16.89 mg, 268.76 μmol) was added. The reaction was further stirred at this temperature for 16 hours monitoring by LC-MS. After completion, the reaction was quenched with water. The reaction mixture was then concentrated under reduced pressure to get the crude product, which was purified by reverse phase preparative HPLC to afford 2-[4-[[4-[4-(2,6-dioxo-3-piperidyl)-2-fluoro-phenyl]-1-piperidyl]methyl]cyclohexyl]-7-isopropoxy-N-pyrazolo[1,5-a]pyrimidin-3-yl-imidazo[1,2-a]pyridine-6-carboxamide (47.5 mg, 63.72 μmol, 47.42% yield) as a yellow solid.

¹H NMR (400 MHz, DMSO-d₆) δ 10.52 (d, J=3.0 Hz, 1H), 9.21 (d, J=3.3 Hz, 1H), 9.10 (d, J=7.0 Hz, 1H), 8.77 (d, J=2.0 Hz, 1H), 8.55 (t, J=1.9 Hz, 1H), 8.13 (s, 1H), 7.75 (d, J=29.8 Hz, 1H), 7.12 (m, 5H), 5.05 (t, J=6.1 Hz, 1H), 3.88 (q, J=5.5 Hz, 1H), 3.53 (d, J=18.2 Hz, 1H), 2.93 (m, 5H), 2.50 (d, J=1.7 Hz, 2H), 2.49-2.08 (m, 13H), 1.55 (m, 8H), 1.15 (m, 2H). LC-MS (ES⁺): m/z 721.32 [M+H]⁺.

### Example 48 Compound of Example 48 was Prepared Substantially Following the Synthesis of Example 47

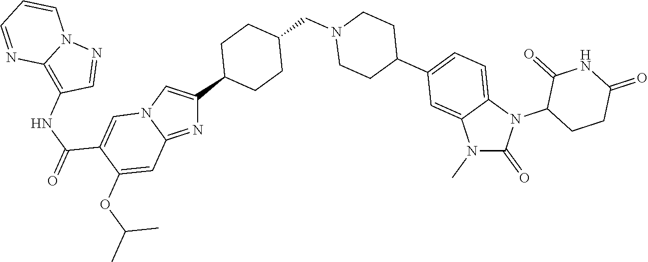 [SMILES: CC(C)Oc1cc2nc([C@H]3CC[C@H](CN4CCC(c5ccc6c(c5)n(C)c(=O)n6C5CCC(=O)NC5=O)CC4)CC3)cn2cc1C(=O)Nc1cnn2cccnc12]

### 2-[4-[[4-[1-(2,6-dioxo-3-piperidyl)-3-methyl-2-oxo-benzimidazol-5-yl]-1-piperidyl]methyl]cyclohexyl]-7-isopropoxy-N-pyrazolo[1,5-a]pyrimidin-3-yl-imidazo[1,2-a]pyridine-6-carboxamide

¹H NMR (400 MHz, DMSO-d₆) δ 11.08 (s, 1H), 10.52 (d, J=2.8 Hz, 1H), 9.18 (d, J=2.9 Hz, 1H), 9.09 (q, J=2.8 Hz, 1H), 8.77 (d, J=1.9 Hz, 1H), 8.54 (q, J=1.8 Hz, 1H), 8.18 (s, 1H), 7.73 (d, J=30.0 Hz, 1H), 7.18 (d, J=12.2 Hz, 1H), 7.11 (s, 1H), 7.06 (q, J=3.7 Hz, 1H), 7.00 (d, J=8.0 Hz, 1H), 6.92 (d, J=8.1 Hz, 1H), 6.55 (s, 1H), 5.33 (q, J=6.0 Hz, 1H), 5.05 (t, J=6.0 Hz, 1H), 3.33 (q, J=14.1 Hz, 3H), 2.91 (q, J=14.1 Hz, 3H), 2.69 (m, 6H), 2.07 (m, 2H), 1.98 (m, 6H), 1.74 (m, 5H), 1.55 (m, 6H), 1.04 (m, 1H). LC-MS (ES⁺): m/z 773.21 [M+H]⁺.

### Example 49 Compound of Example 49 was Prepared Substantially Following the Synthesis of Example 47

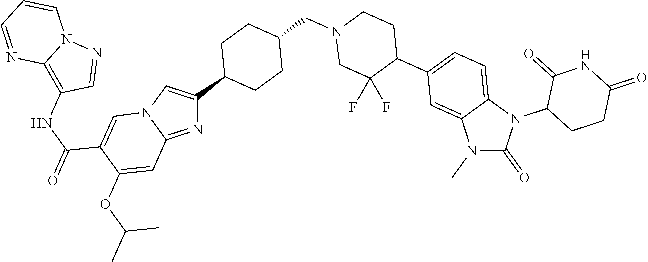 [SMILES: CC(C)Oc1cc2nc([C@H]3CC[C@H](CN4CCC(c5ccc6c(c5)n(C)c(=O)n6C5CCC(=O)NC5=O)C(F)(F)C4)CC3)cn2cc1C(=O)Nc1cnn2cccnc12]

### 2-[4-[[4-[1-(2,6-dioxo-3-piperidyl)-3-methyl-2-oxo-benzimidazol-5-yl]-3,3-difluoro-1-piperidyl]methyl]cyclohexyl]-7-isopropoxy-N-pyrazolo[1,5-a]pyrimidin-3-yl-imidazo[1,2-a]pyridine-6-carboxamide

¹H NMR (400 MHz, DMSO-d₆) δ 11.09 (s, 1H), 10.52 (d, J=2.8 Hz, 1H), 9.19 (d, J=3.1 Hz, 1H), 9.09 (q, J=2.8 Hz, 1H), 8.77 (d, J=2.0 Hz, 1H), 8.54 (q, J=1.7 Hz, 1H), 8.37 (s, 1H), 7.74 (d, J=31.5 Hz, 1H), 7.18 (t, J=7.4 Hz, 2H), 7.07-6.74 (m, 2H), 5.36 (q, J=6.0 Hz, 1H), 5.05 (m, 1H), 3.34 (m, 4H), 3.00 (m, 3H), 2.85 (m, 1H), 2.60 (m, 1H), 2.31 (q, J=6.1 Hz, 2H), 2.13 (m, 9H), 1.98 (q, J=13.5 Hz, 1H), 1.77 (m, 1H), 1.54 (m, 8H), 1.43 (t, J=11.8 Hz, 1H), 1.05 (t, J=5.6 Hz, 1H). LC-MS (ES⁺): m/z 809.16 [M+H]⁺.

### Example 50 Compound of Example 50 was Prepared Substantially Following the Synthesis of Example 47

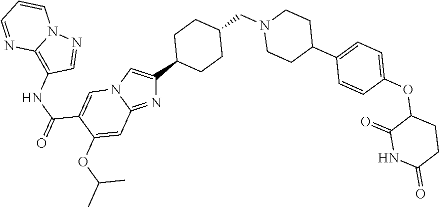 [SMILES: CC(C)Oc1cc2nc([C@H]3CC[C@H](CN4CCC(c5ccc(OC6CCC(=O)NC6=O)cc5)CC4)CC3)cn2cc1C(=O)Nc1cnn2cccnc12]

### 2-[4-[[4-[4-[(2,6-dioxo-3-piperidyl)oxy]phenyl]-1-piperidyl]methyl]cyclohexyl]-7-isopropoxy-N-pyrazolo[1,5-a]pyrimidin-3-yl-imidazo[1,2-a]pyridine-6-carboxamide

¹H NMR (400 MHz, DMSO-d₆) δ 10.91 (s, 1H), 10.52 (d, J=2.5 Hz, 1H), 9.18 (d, J=4.5 Hz, 1H), 9.09 (q, J=2.8 Hz, 1H), 8.77 (d, J=1.8 Hz, 1H), 8.54 (q, J=1.8 Hz, 1H), 8.30 (s, 2H), 7.73 (d, J=31.1 Hz, 1H), 7.17 (q, J=7.1 Hz, 3H), 7.06 (q, J=3.7 Hz, 2H), 6.93 (t, J=4.3 Hz, 1H), 5.14 (q, J=5.3 Hz, 1H), 5.04 (q, J=6.0 Hz, 1H), 2.94 (d, J=10.6 Hz, 3H), 2.64 (m, 1H), 2.56 (m, 1H), 2.42 (m, 1H), 2.13 (m, 6H), 1.92 (t, J=11.1 Hz, 3H), 1.54 (m, 13H), 1.03 (d, J=12.5 Hz, 1H). LC-MS (ES⁺): m/z 719.43 [M+H]⁺.

### Example 51 Compound of Example 51 was Prepared Substantially Following the Synthesis of Example 47

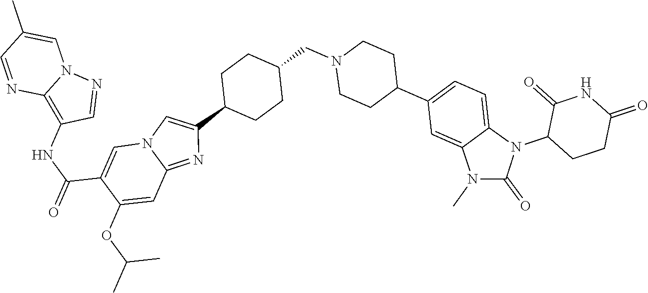 [SMILES: Cc1cnc2c(NC(=O)c3cn4cc([C@H]5CC[C@H](CN6CCC(c7ccc8c(c7)n(C)c(=O)n8C7CCC(=O)NC7=O)CC6)CC5)nc4cc3OC(C)C)cnn2c1]

### 2-[4-[[4-[1-(2,6-dioxo-3-piperidyl)-3-methyl-2-oxo-benzimidazol-5-yl]-1-piperidyl]methyl]cyclohexyl]-7-isopropoxy-N-(6-methylpyrazolo[1,5-a]pyrimidin-3-yl)imidazo[1,2-a]pyridine-6-carboxamide

¹H NMR (400 MHz, DMSO-d₆) δ 11.08 (s, 1H), 10.50 (d, J=2.7 Hz, 1H), 9.17 (d, J=3.0 Hz, 1H), 8.93 (s, 1H), 8.67 (d, J=1.9 Hz, 1H), 8.46 (d, J=1.7 Hz, 1H), 8.22 (s, 1H), 7.73 (d, J=30.1 Hz, 1H), 7.15 (t, J=15.6 Hz, 2H), 6.96 (q, J=13.8 Hz, 2H), 5.33 (q, J=6.0 Hz, 1H), 5.04 (m, 1H), 3.34 (s, 3H), 2.90 (q, J=13.2 Hz, 4H), 2.67 (m, 4H), 2.34 (s, 3H), 2.06 (m, 8H), 1.63 (m, 9H), 1.42 (m, 4H), 1.05 (t, J=10.8 Hz, 1H). LC-MS (ES⁺): m/z 787.43 [M+H]⁺.

### Example 52 Compound of Example 52 was Prepared Substantially Following the Synthesis of Example 47

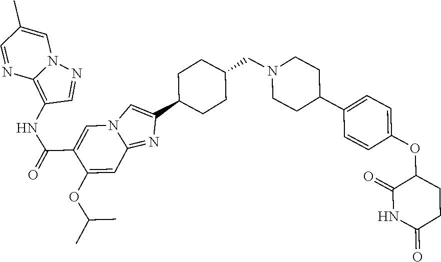 [SMILES: Cc1cnc2c(NC(=O)c3cn4cc([C@H]5CC[C@H](CN6CCC(c7ccc(OC8CCC(=O)NC8=O)cc7)CC6)CC5)nc4cc3OC(C)C)cnn2c1]

### 2-[4-[[4-[4-[(2,6-dioxo-3-piperidyl)oxy]phenyl]-1-piperidyl]methyl]cyclohexyl]-7-isopropoxy-N-(6-methylpyrazolo[1,5-a]pyrimidin-3-yl)imidazo[1,2-a]pyridine-6-carboxamide

¹H NMR (400 MHz, DMSO-d₆) δ 10.92 (s, 1H), 10.50 (d, J=2.8 Hz, 1H), 9.20 (d, J=3.9 Hz, 1H), 8.94 (t, J=0.9 Hz, 1H), 8.67 (d, J=2.0 Hz, 1H), 8.46 (d, J=1.9 Hz, 1H), 8.13 (s, 1H), 7.78 (s, 1H), 7.71 (s, 1H), 7.17 (d, J=13.8 Hz, 3H), 6.98 (q, J=3.6 Hz, 2H), 6.52 (s, 1H), 5.16 (q, J=5.2 Hz, 1H), 5.03 (s, 1H), 3.31 (m, 2H), 2.75 (m, 5H), 2.50 (m, 2H), 2.34 (s, 3H), 1.92 (m, 10H), 1.54 (t, J=3.0 Hz, 8H), 1.17 (m, 1H). LC-MS (ES⁺): m/z 733.46 [M+H]⁺.

### Example 53 Compound of Example 53 was Prepared Substantially Following the Synthesis of Example 47

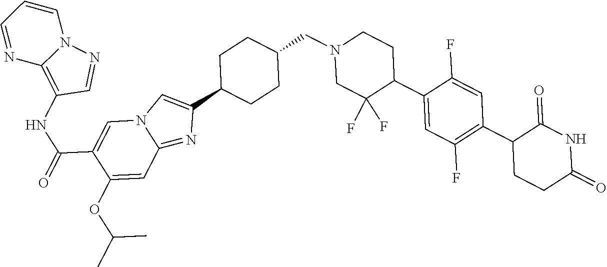 [SMILES: CC(C)Oc1cc2nc([C@H]3CC[C@H](CN4CCC(c5cc(F)c(C6CCC(=O)NC6=O)cc5F)C(F)(F)C4)CC3)cn2cc1C(=O)Nc1cnn2cccnc12]

### 2-[4-[[4-[4-(2,6-dioxo-3-piperidyl)-2,5-difluoro-phenyl]-3,3-difluoro-1-piperidyl]methyl]cyclohexyl]-7-isopropoxy-N-pyrazolo[1,5-a]pyrimidin-3-yl-imidazo[1,2-a]pyridine-6-carboxamide

¹H NMR (400 MHz, DMSO-d₆) δ 10.92 (s, 1H), 10.52 (d, J=2.6 Hz, 1H), 9.18 (d, J=4.4 Hz, 1H), 9.09 (q, J=2.8 Hz, 1H), 8.77 (d, J=1.9 Hz, 1H), 8.54 (q, J=1.8 Hz, 1H), 8.33 (s, 1H), 7.74 (d, J=32.3 Hz, 1H), 7.28 (m, 2H), 7.18 (d, J=11.0 Hz, 1H), 7.06 (q, J=3.7 Hz, 1H), 5.05 (m, 1H), 4.07 (q, J=5.7 Hz, 1H), 3.32 (m, 2H), 2.80 (m, 2H), 2.60 (m, 1H), 2.50 (m, 1H), 2.34 (m, 1H), 2.04 (m, 7H), 1.91 (d, J=6.0 Hz, 2H), 1.74 (d, J=6.0 Hz, 2H), 1.59 (m, 7H), 1.54 (d, J=6.0 Hz, 2H), 1.04 (d, J=11.0 Hz, 1H). LC-MS (ES⁺): m/z 775.42 [M+H]⁺.

### Example 54 Compound of Example 54 was Prepared Substantially Following the Synthesis of Example 47

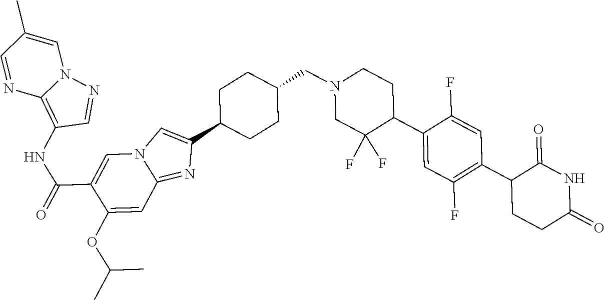 [SMILES: Cc1cnc2c(NC(=O)c3cn4cc([C@H]5CC[C@H](CN6CCC(c7cc(F)c(C8CCC(=O)NC8=O)cc7F)C(F)(F)C6)CC5)nc4cc3OC(C)C)cnn2c1]

### 2-[4-[[4-[4-(2,6-dioxo-3-piperidyl)-2,5-difluoro-phenyl]-3,3-difluoro-1-piperidyl]methyl]cyclohexyl]-7-isopropoxy-N-(6-methylpyrazolo[1,5-a]pyrimidin-3-yl)imidazo[1,2-a]pyridine-6-carboxamide

¹H NMR (400 MHz, DMSO-d₆) δ 10.92 (s, 1H), 10.50 (s, 1H), 9.17 (d, J=4.4 Hz, 1H), 8.93 (s, 1H), 8.67 (d, J=1.9 Hz, 1H), 8.47 (m, 3H), 7.74 (d, J=32.3 Hz, 1H), 7.24 (m, 2H), 7.16 (m, 1H), 5.04 (t, J=6.0 Hz, 1H), 4.08 (q, J=5.7 Hz, 1H), 3.37 (m, 2H), 3.16 (m, 1H), 2.92 (m, 1H), 2.67 (m, 1H), 2.50 (m, 2H), 2.37 (m, 1H), 2.26 (m, 6H), 2.03 (m, 3H), 1. 91 (m, 2H), 1.79 (m, 2H), 1.54 (m, 7H), 1.42 (m, 1H), 1.04 (d, J=10.9 Hz, 1H). LC-MS (ES⁺): m/z 291.23 [M+H]⁺.

### Synthesis of methyl 2-(1-tert-butoxycarbonyl-4-piperidyl)-6-isopropoxy-indazole-5-carboxylate

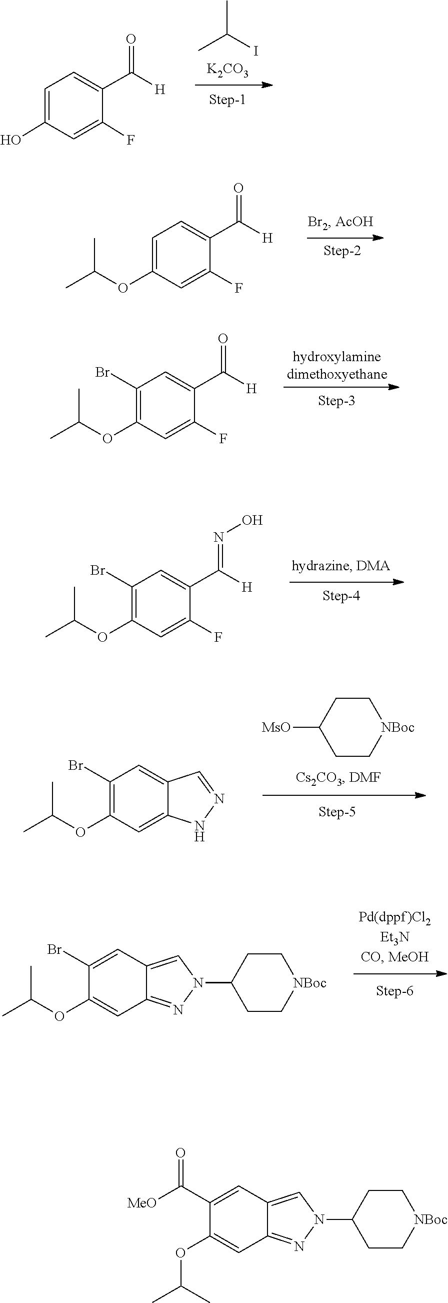

### Step-1:

To a solution of 2-fluoro-4-hydroxy-benzaldehyde (20.00 g, 142.74 mmol) in DMF (200 mL) was added potassium carbonate (39.46 g, 285.49 mmol) and 2-iodopropane (26.69 g, 157.02 mmol, 15.70 mL). The reaction mixture was stirred at 80° C. for 16 hours. The reaction mixture was then diluted with water (2000 mL) and extracted with ethyl acetate (500 mL×3). The combined organic layers were washed with brine (1000 mL) and concentrated under reduced pressure. The resulting residue was purified by column chromatography (silica gel, petroleum ether/ethyl acetate=20/1 to 5/1) to afford 2-fluoro-4-isopropoxy-benzaldehyde (22 g, 120.62 mmol, 84.50% yield) as a colorless oil. LC-MS (ES⁺): m/z 183.1 [M+H]⁺.

### Step-2:

To a solution of 2-fluoro-4-isopropoxy-benzaldehyde (40 g, 219.55 mmol) in acetic acid (800 mL) was added a solution of molecular bromine (38.59 g, 241.50 mmol) in acetic acid (40 mL) dropwise at 20° C. The reaction mixture was stirred at 50° C. for 16 hours. After consumption of the reactant as shown by TLC and LC-MS, the mixture was filtered and concentrated under reduced pressure to give 5-bromo-2-fluoro-4-isopropoxy-benzaldehyde (50 g, 147.46 mmol, 67.16% yield) as a yellow oil, which was used in the next step without purification. LC-MS (ES⁺): m/z 260.9 [M+H]⁺.

### Step-3:

To a solution of 5-bromo-2-fluoro-4-isopropoxy-benzaldehyde (50 g, 124.48 mmol) in ethanol (500 mL) were added hydroxylamine hydrochloride (8.65 g, 124.48 mmol, 5.18 mL) and potassium carbonate (18.92 g, 136.93 mmol). The reaction mixture was stirred at 100° C. for 2 hours. After consumption of the reactant as shown by LC-MS, the reaction mixture was filtered and the filtrate was concentrated under reduce pressure to give the crude product, which was used for next step directly without purification. Compound (E)-5-bromo-2-fluoro-4-isopropoxybenzaldehyde oxime (50 g, 139.44 mmol, 112.02% yield) was obtained as a yellow oil. LC-MS (ES⁺): m/z 276.0 [M+H]⁺.

### Step-4:

To a solution of (E)-5-bromo-2-fluoro-4-isopropoxy-benzaldehyde oxime (50 g, 181.09 mmol) in DMA (500 mL) was added hydrazine hydrate (96.97 g, 1.94 mol, 94.15 mL). The reaction mixture was stirred at 140° C. for 16 hours. After consumption of the reactant as confirmed by LC-MS, the reaction mixture was diluted with H₂O (1000 mL) and extracted with ethyl acetate (1000 mL×3). The combined organic layers were washed with aqueous NaCl (1000 mL×2), dried over Na₂SO₄, filtered, and the filtrate evaporated to dryness. The residue was purified by column chromatography (silica gel, petroleum ether/ethyl acetate=10/1 to 3/1) to give 5-bromo-6-isopropoxy-1H-indazole (17 g, 48.65 mmol, 26.86% yield) as a yellow oil. LC-MS (ES⁺): 254.9 m/z [M+H]⁺.

### Step-5:

To a solution of 5-bromo-6-isopropoxy-1H-indazole (14 g, 54.88 mmol) in DMF (150 mL) was added dicesium carbonate (35.76 g, 109.76 mmol) and tert-butyl 4-((methylsulfonyl)oxy)piperidine-1-carboxylate (19.93 g, 71.34 mmol). The reaction mixture was stirred at 80° C. for 16 hours. LCMS showed the reaction was consumed completely and several new peaks were seen in LCMS with 27% of desired compound detected. The reaction mixture was diluted with H2O (200 mL) and extracted with EA (200 mL*3). The combined organic layers were washed with brine (200 mL*2), dried over Na₂SO₄, filtered, and concentrated in vacuum to dryness. The residue was purified by prep-HPLC (Biotage Isolera One, I.D.95 mm×H365 mm Welch Ultimate XB_C18 20-40 μm; 120 A Mobile phase, MeCN/H₂O, Gradient B %,30-80% 30 min;80% 25 min). Compound tert-butyl 4-(5-bromo-6-isopropoxy-2H-indazol-2-yl)piperidine-1-carboxylate (2.1 g, 4.75 mmol, 8.66% yield) was obtained as a white solid. LC-MS (ES⁺): m/z 438.2 [M+H]⁺.

### Step-6:

To a solution of bromo tert-butyl 4-(5-bromo-6-isopropoxy-indazol-2-yl)piperidine-1-carboxylate (2.00 g, 4.55 mmol) in methanol (75 mL) was added N,N-diethylethanamine (7.48 g, 73.95 mmol, 10.31 mL) and cyclopentyl(diphenyl)phosphane;dichloropalladium;iron (633.54 mg, 865.85 μmol). Then the mixture was stirred at 80° C. for 22 hours under carbon monoxide (50 Psi). After consumption of the reactant as shown by TLC, the reaction mixture was filtered, washed with ethyl acetate. The filtrate was then concentrated under reduced pressure and purified by column chromatography (silica gel, petroleum ether/ethyl acetate=50/1 to 1/1) to afford methyl 2-(1-tert-butoxycarbonyl-4-piperidyl)-6-isopropoxy-indazole-5-carboxylate (1.0 g, 2.40 mmol, 52.62% yield) as a yellow solid, which was used in the next step directly. LC-MS (ES⁺): m/z 418.1 [M+H]⁺.

### Synthesis of 6-isopropoxy-2-(4-piperidyl)-N-pyrazolo[1,5-a]pyrimidin-3-yl-indazole-5-carboxamide

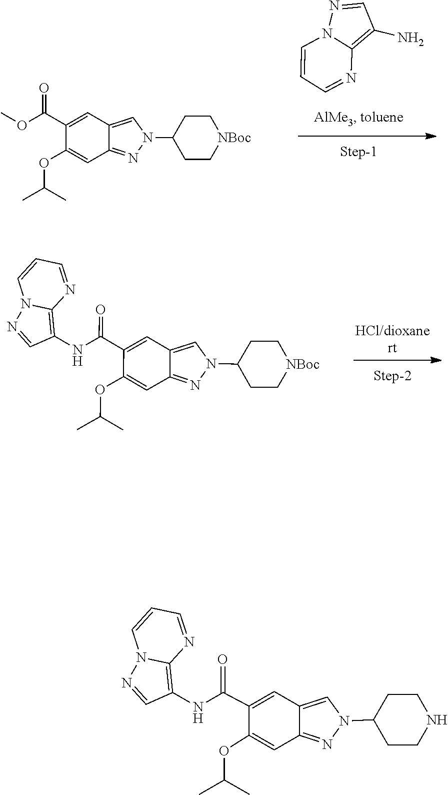 [SMILES: COC(=O)c1cc2cn(C3CCN(C(=O)OC(C)(C)C)CC3)nc2cc1OC(C)C ; Nc1cnn2cccnc12 ; CC(C)Oc1cc2nn(C3CCN(C(=O)OC(C)(C)C)CC3)cc2cc1C(=O)Nc1cnn2cccnc12 ; CC(C)Oc1cc2nn(C3CCNCC3)cc2cc1C(=O)Nc1cnn2cccnc12]

### Step-1:

A solution of pyrazolo[1,5-a]pyrimidin-3-amine (321.29 mg, 2.40 mmol) in toluene (5 mL) was degassed and purged with N₂ three times, and then the mixture was stirred at 25° C. for 0.5 hour under N₂ atmosphere. After that, it was added a solution of methyl 2-(1-(tert-butoxycarbonyl)piperidin-4-yl)-6-isopropoxy-2H-indazole-5-carboxylate (1 g, 2.40 mmol) in toluene (5 mL). After TLC indicated the complete consumption of the reactant, the reaction mixture was quenched by addition of NaHCO₃ solution (20 mL) at 0° C., and the mixture was diluted with ethyl acetate (20 mL) and extracted with ethyl acetate (50 mL×3). The combined organic layers were washed with brine (30 mL), dried over anhydrous sodium sulfate, filtered, and concentrated under reduced pressure. The residue was purified by flash column chromatography (ISCO®; 40 g SepaFlash® Silica Flash Column, 0-10% DCM/MeOH as eluent at 30 mL/min). Compound tert-butyl 4-(6-isopropoxy-5-(pyrazolo[1,5-a]pyrimidin-3-ylcarbamoyl)-2H-indazol-2-yl)piperidine-1-carboxylate (0.93 g, 1.49 mmol, 62.02% yield) was obtained as an orange solid. LC-MS (ES⁺): m/z 520.3[M+H]⁺.

### Step-2:

To a stirred solution of tert-butyl 4-[6-isopropoxy-5-(pyrazolo[1,5-a]pyrimidin-3-ylcarbamoyl) indazol-2-yl]piperidine-1-carboxylate (0.93 g, 1.79 mmol) in dioxane (5 mL) was added with HCl (4 M, 4.47 mL). After completion of the reaction as confirmed by LC-MS, the solvent was removed by vacuum and the crude product was triturated with ether to afford 6-isopropoxy-2-(4-piperidyl)-N-pyrazolo[1,5-a]pyrimidin-3-yl-indazole-5-carboxamide HCl salt (800 mg, 1.42 mmol, 79.41% yield) as a yellow solid. LC-MS (ES⁺): m/z 420.2 [M+H]⁺.

### Example 55

### Synthesis of 2-(1-(2-(4-(4-((2,6-dioxopiperidin-3-yl)amino)phenyl)piperidin-1-yl)acetyl)piperidin-4-yl)-6-isopropoxy-N-(pyrazolo[1,5-a]pyrimidin-3-yl)-2H-indazole-5-carboxamide

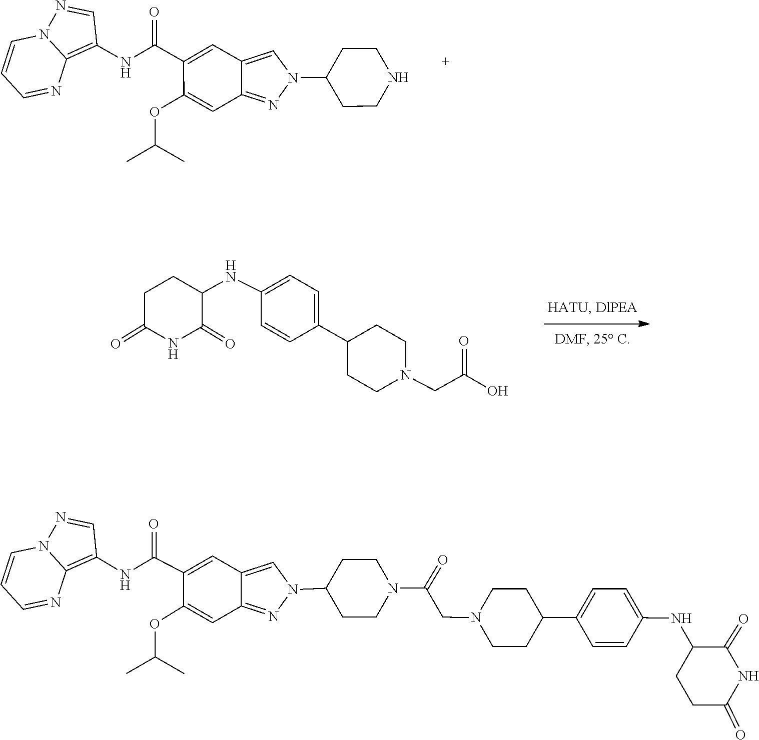 [SMILES: CC(C)Oc1cc2nn(C3CCNCC3)cc2cc1C(=O)Nc1cnn2cccnc12 ; O=C(O)CN1CCC(c2ccc(NC3CCC(=O)NC3=O)cc2)CC1 ; CC(C)Oc1cc2nn(C3CCN(C(=O)CN4CCC(c5ccc(NC6CCC(=O)NC6=O)cc5)CC4)CC3)cc2cc1C(=O)Nc1cnn2cccnc12]

To a solution of 6-isopropoxy-2-(4-piperidyl)-N-pyrazolo[1,5-a]pyrimidin-3-yl-indazole-5-carboxamide (120 mg, 263.19 μmol) and 2-(4-(4-((2,6-dioxopiperidin-3-yl)amino)phenyl)piperidin-1-yl)acetic acid (201.00 mg, 526.39 μmol) in DMF (2 mL) was added DIPEA (340.16 mg, 2.63 mmol, 458.43 μL) and HATU (200.15 mg, 526.39 μmol). The mixture was stirred at 25° C. for 24 hours. LC-MS showed reagent were consumed completely and the desired mass was detected. The reaction was directly purified by prep-HPLC to give 2-(1-(2-(4-(4-((2,6-dioxopiperidin-3-yl)amino)phenyl)piperidin-1-yl)acetyl)piperidin-4-yl)-6-isopropoxy-N-(pyrazolo[1,5-a]pyrimidin-3-yl)-2H-indazole-5-carboxamide (70.11 mg, 79.17 μmol, 30.08% yield) as a yellow solid. ¹H NMR (400 MHz, DMSO-d₆) δ ppm 10.77-10.82 (m, 1H) 10.75 (s, 1H) 9.44-9.59 (m, 1H) 9.09 (dd, J=7.2, 1.6 Hz, 1H) 8.81 (s, 1H) 8.65 (s, 1H) 8.62 (s, 1H) 8.54 (dd, J=4.0, 1.6 Hz, 1H) 7.27 (s, 1H) 7.03-7.08 (m, 1H) 6.99 (d, J=8.8 Hz, 2H) 6.66 (d, J=8.4 Hz, 2H) 5.01-5.10 (m, 1H) 4.80-4.92 (m, 1H) 4.33-4.60 (m, 4H) 4.30 (br dd, J=11.2, 4.4 Hz, 1H) 3.83 (br d, J=14.0 Hz, 1H) 3.59 (br d, J=11.2 Hz, 1H) 3.34 (t, J=11.2 Hz, 2H) 3.07-3.14 (m, 1H) 2.91-3.04 (m, 1H) 2.64-2.80 (m, 2H) 2.60 (dt, J=17.6, 4.0 Hz, 1H) 2.20-2.30 (m, 2H) 2.06-2.19 (m, 2H) 1.80-2.06 (m, 6H) 1.56 (d, J=6.0 Hz, 6H). LC-MS (ES⁺): m/z 747.3 [M+H]⁺.

### Example 56 Compound of Example 56 was Prepared Substantially Following the Synthesis of Example 55

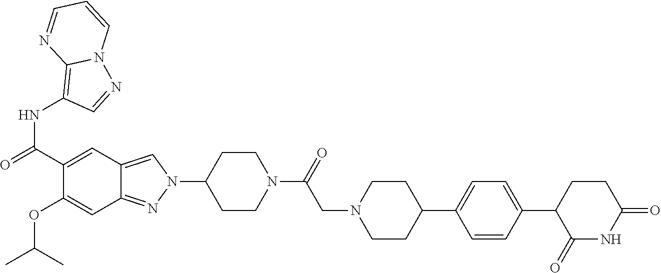

### 2-(1-(2-(4-(4-(2,6-dioxopiperidin-3-yl)phenyl)piperidin-1-yl)acetyl)piperidin-4-yl)-6-isopropoxy-N-(pyrazolo[1,5-a]pyrimidin-3-yl)-2H-indazole-5-carboxamide

¹H-NMR: (400 MHz, DMSO-d₆) δ ppm 10.84 (s, 1H) 10.74 (s, 1H) 9.56 (br s, 1H) 9.08 (dd, J=7.2, 1.6 Hz, 1H) 8.80 (s, 1H) 8.65 (s, 1H) 8.61 (s, 1H) 8.53 (dd, J=3.6, 1.2 Hz, 1H) 7.24-7.36 (m, 2H) 7.19-7.24 (m, 3H) 7.02-7.07 (m, 1H) 4.94-5.13 (m, 1H) 4.79-4.91 (m, 1H) 4.48-4.60 (m, 2H) 4.38-4.45 (m, 1H) 3.77-3.96 (m, 2H) 3.62 (br d, J=11.2 Hz, 2H) 3.28-3.43 (m, 2H) 3.06-3.22 (m, 2H) 2.99 (br t, J=13.2 Hz, 1H) 2.80-2.92 (m, 1H) 2.63-2.72 (m, 1H) 2.09-2.29 (m, 5H) 1.97-2.09 (m, 5H) 1.55 (d, J=5.99 Hz, 6H). LC-MS (ES⁺): m/z 732.5 [M+H]⁺.

### Example 57 Compound of Example 57 was Prepared Substantially Following the Synthesis of Example 55

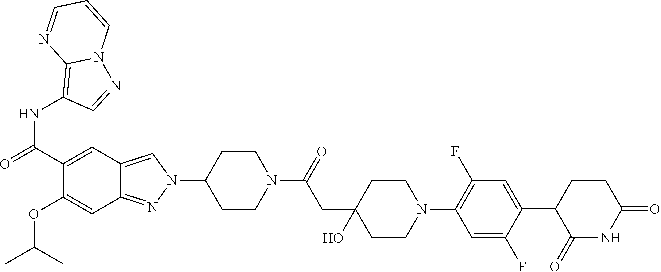 [SMILES: CC(C)Oc1cc2nn(C3CCN(C(=O)CC4(O)CCN(c5cc(F)c(C6CCC(=O)NC6=O)cc5F)CC4)CC3)cc2cc1C(=O)Nc1cnn2cccnc12]

### 2-(1-(2-(1-(4-(2,6-dioxopiperidin-3-yl)-2,5-difluorophenyl)-4-hydroxypiperidin-4-yl)acetyl)piperidin-4-yl)-6-isopropoxy-N-(pyrazolo[1,5-a]pyrimidin-3-yl)-2H-indazole-5-carboxamide

¹H-NMR: (400 MHz, DMSO-d₆) δ=10.85 (s, 1H), 10.75 (s, 1H), 9.07 (dd, J=1.2, 7.0 Hz, 1H), 8.80 (s, 1H), 8.62 (d, J=4.8 Hz, 2H), 8.53 (dd, J=1.2, 4.0 Hz, 1H), 7.30 (s, 1H), 7.14-7.07 (m, 1H), 7.04 (dd, J=4.0, 7.0 Hz, 1H), 6.87 (dd, J=7.4, 12.0 Hz, 1H), 5.08-4.97 (m, 1H), 4.85-4.71 (m, 1H), 4.61 (br d, J=12.4 Hz, 1H), 4.27-4.15 (m, 1H), 3.96 (br dd, J=4.8, 12.8 Hz, 2H), 3.31-3.23 (m, 1H), 3.14 (br d, J=10.4 Hz, 2H), 3.08-2.98 (m, 2H), 2.82 (br t, J=12.8 Hz, 1H), 2.78-2.71 (m, 1H), 2.67 (dt, J=2.4, 4.0 Hz, 1H), 2.61 (br d, J=5.2 Hz, 2H), 2.24-2.13 (m, 3H), 2.08 (br dd, J=3.6, 10.8 Hz, 1H), 2.00-1.90 (m, 2H), 1.83-1.67 (m, 4H), 1.55 (d, J=6.0 Hz, 6H). LC-MS (ES⁺): m/z 784.3 [M+H]⁺.

### Synthesis of 6-isopropoxy-2-(4-piperidyl)-N-[6-(trifluoromethyl)-2-pyridyl]indazole-5-carboxamide

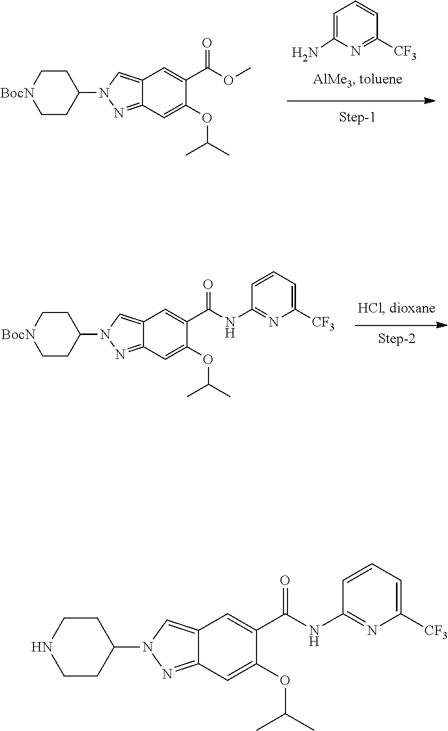 [SMILES: COC(=O)c1cc2cn(C3CCN(C(=O)OC(C)(C)C)CC3)nc2cc1OC(C)C ; Nc1cccc(C(F)(F)F)n1 ; CC(C)Oc1cc2nn(C3CCN(C(=O)OC(C)(C)C)CC3)cc2cc1C(=O)Nc1cccc(C(F)(F)F)n1 ; CC(C)Oc1cc2nn(C3CCNCC3)cc2cc1C(=O)Nc1cccc(C(F)(F)F)n1]

### Step-1:

A solution of 6-(trifluoromethyl)pyridin-2-amine (194.15 mg, 1.20 mmol) in toluene (8 mL) was added trimethylalumane (2 M, 598.81 μL) at 25° C. After stirring for 30 minutes, methyl 2-(1-tert-butoxycarbonyl-4-piperidyl)-6-isopropoxy-indazole-5-carboxylate (0.5 g, 1.20 mmol) was added and the mixture was stirred at 120° C. for 4 hours under N₂ atmosphere. After completion of the reaction as confirmed by LC-MS, the reaction mixture was quenched by addition of NH₄Cl solution (20 mL) at 25° C. The mixture was then diluted with water (50 mL) and extracted with ethyl acetate (20 mL×4). The combined organic layers were washed with brine (15 mL×2), dried over anhydrous sodium sulfate, filtered and concentrated under reduced pressure. The residue was purified by flash column chromatography (ISCO®; 12 g SepaFlash® Silica Flash Column, 10-100% ethyl acetate/petroleum ether gradient as eluent at 60 mL/min). Compound tert-butyl 4-[6-isopropoxy-5-[[6-(trifluoromethyl)-2-pyridyl]carbamoyl]indazol-2-yl]piperidine-1-carboxylate (500 mg, 849.21 μmol, 70.91% yield) was obtained as a white solid. LC-MS (ES⁺): m/z 548.3 [M+H]⁺.

### Step-2:

To a stirred solution of tert-butyl 4-[6-isopropoxy-5-[[6-(trifluoromethyl)-2-pyridyl]carbamoyl]indazol-2-yl]piperidine-1-carboxylate (500 mg, 913.13 μmol) in DCM (3 mL) was added HCl in dioxane (4 M, 1.14 mL). After completion of the reaction as confirmed by LC-MS, the reaction mixture was concentrated in vacuo and the crude product was triturated with ether to afford 6-isopropoxy-2-(4-piperidyl)-N-[6-(trifluoromethyl)-2-pyridyl]indazole-5-carboxamide (400 mg, 859.35 μmol, 94.11% yield) as a white solid. LC-MS (ES⁺): m/z 448.2 [M+H]⁺.

### Example 58 Synthesis of 2-[1-[2-[1-[4-[(2,6-dioxo-3-piperidyl)amino]phenyl]-4-hydroxy-4-piperidyl]acetyl]-4-piperidyl]-6-isopropoxy-N-[6-(trifluoromethyl)-2-pyridyl]indazole-5-carboxamide

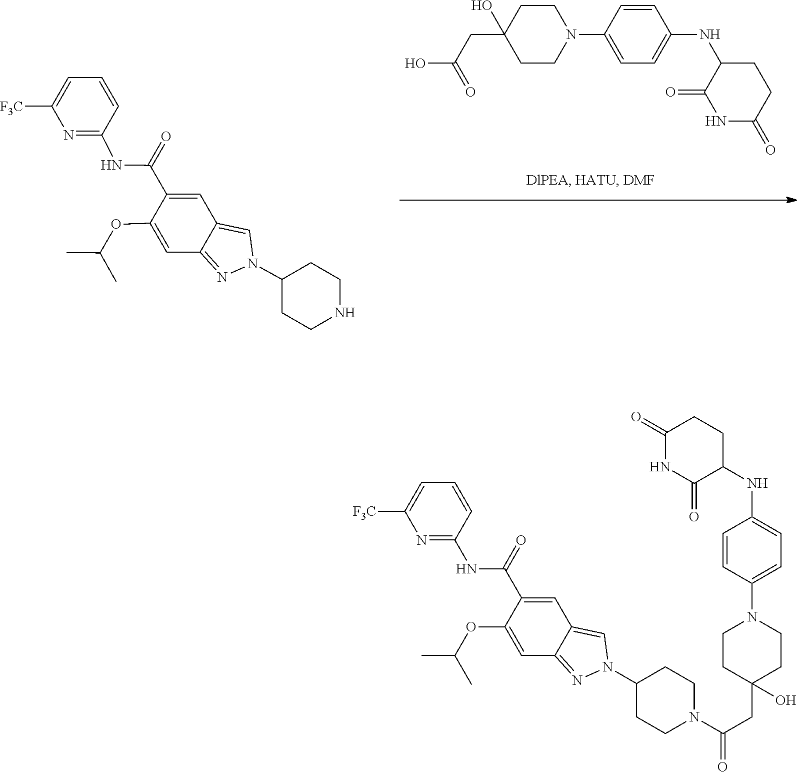 [SMILES: CC(C)Oc1cc2nn(C3CCNCC3)cc2cc1C(=O)Nc1cccc(C(F)(F)F)n1 ; O=C(O)CC1(O)CCN(c2ccc(NC3CCC(=O)NC3=O)cc2)CC1 ; CC(C)Oc1cc2nn(C3CCN(C(=O)CC4(O)CCN(c5ccc(NC6CCC(=O)NC6=O)cc5)CC4)CC3)cc2cc1C(=O)Nc1cccc(C(F)(F)F)n1]

To a solution of 2-[1-[4-[(2,6-dioxo-3-piperidyl)amino]phenyl]-4-hydroxy-4-piperidyl]acetic acid (33.65 mg, 93.12 μmol) in DMF (0.5 mL) was added HATU (53.11 mg, 139.68 μmol) and DIPEA (48.14 mg, 372.48 μmol, 64.88 μL). The mixture was stirred at 25° C. for 0.5 hour, and 6-isopropoxy-2-(4-piperidyl)-N-[6-(trifluoromethyl)-2-pyridyl]indazole-5-carboxamide (50 mg, 111.74 μmol) was added and stirred at 25° C. for another 2.5 hours. After complete consumption of the reactant as shown by LC-MS, the residue was purified by prep-HPLC (ACSWH-GX-O/3_Phenomenex Luna C18 75×30 mm×3 um, water (0.1% TFA) and acetonitrile; Gradient: 35-65% acetonitrile) to afford 2-[1-[2-[1-[4-[(2,6-dioxo-3-piperidyl)amino]phenyl]-4-hydroxy-4-piperidyl]acetyl]-4-piperidyl]-6-isopropoxy-N-[6-(trifluoromethyl)-2-pyridyl]indazole-5-carboxamide (19.72 mg, 21.79 μmol, 23.40% yield) was obtained as a green solid. ¹H NMR (400 MHz, DMSO-d₆) δ=11.19 (s, 1H), 10.85 (s, 1H), 8.64-8.59 (m, 1H), 8.56-8.49 (m, 2H), 8.15 (t, J=8.0 Hz, 1H), 7.65 (d, J=7.7 Hz, 1H), 7.40 (br d, J=7.8 Hz, 2H), 7.25 (s, 1H), 6.77 (br d, J=9.0 Hz, 2H), 6.50-6.30 (m, 1H), 5.52-5.13 (m, 1H), 5.00-4.90 (m, 1H), 4.86-4.74 (m, 1H), 4.66-4.56 (m, 1H), 4.47-4.35 (m, 1H), 4.27-4.14 (m, 1H), 3.72-3.61 (m, 3H), 3.42 (br d, J=9.0 Hz, 2H), 3.29 (br t, J=11.7 Hz, 1H), 2.90-2.59 (m, 4H), 2.23-2.04 (m, 6H), 1.99-1.88 (m, 4H), 1.46 (s, 3H), 1.44 (s, 3H). LC-MS (ES⁺): m/z 791.4 [M+H]⁺.

### Example 59 Compound of Example 59 was Prepared Substantially Following the Synthesis of Example 58

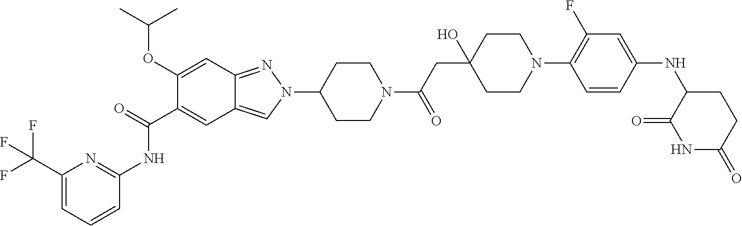 [SMILES: CC(C)Oc1cc2nn(C3CCN(C(=O)CC4(O)CCN(c5ccc(NC6CCC(=O)NC6=O)cc5F)CC4)CC3)cc2cc1C(=O)Nc1cccc(C(F)(F)F)n1]

### 2-[1-[2-[1-[4-[(2,6-dioxo-3-piperidyl)amino]-2-fluoro-phenyl]-4-hydroxy-4-piperidyl]acetyl]-4-piperidyl]-6-isopropoxy-N-[6-(trifluoromethyl)-2-pyridyl]indazole-5-carboxamide

¹H NMR (400 MHz, DMSO-d₆) δ=11.19 (s, 1H), 10.84 (s, 1H), 8.61 (s, 1H), 8.55-8.49 (m, 2H), 8.15 (t, J=8.1 Hz, 1H), 7.65 (d, J=7.7 Hz, 1H), 7.26 (s, 1H), 6.68-6.47 (m, 2H), 4.95 (td, J=6.1, 12.1 Hz, 1H), 4.86-4.74 (m, 1H), 4.66-4.56 (m, 1H), 4.42-4.31 (m, 1H), 4.25-4.15 (m, 2H), 3.35-3.22 (m, 2H), 3.15-2.99 (m, 1H), 2.88-2.57 (m, 5H), 2.24-1.56 (m, 13H), 1.45 (d, J=6.0 Hz, 6H). LC-MS (ES⁺): m/z 809.4 [M+H]⁺.

### Example 60 Compound of Example 60 was Prepared Substantially Following the Synthesis of Example 58

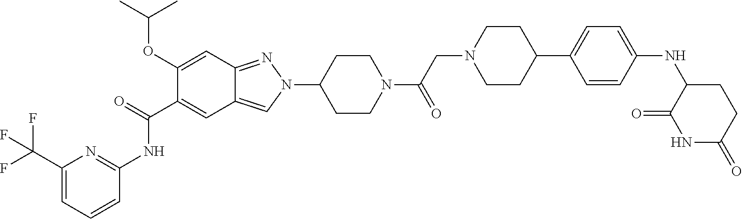 [SMILES: CC(C)Oc1cc2nn(C3CCN(C(=O)CN4CCC(c5ccc(NC6CCC(=O)NC6=O)cc5)CC4)CC3)cc2cc1C(=O)Nc1cccc(C(F)(F)F)n1]

### 2-(1-(2-(4-(4-((2,6-dioxopiperidin-3-yl)amino)phenyl)piperidin-1-yl)acetyl)piperidin-4-yl)-6-isopropoxy-N-(6-(trifluoromethyl)pyridin-2-yl)-2H-indazole-5-carboxamide

¹H NMR (400 MHz, DMSO-d₆) δ=11.18 (s, 1H), 10.83 (br s, 1H), 8.61 (s, 1H), 8.56-8.50 (m, 2H), 8.14 (t, J=8.1 Hz, 1H), 7.64 (d, J=7.5 Hz, 1H), 7.31-7.22 (m, 2H), 7.07-6.96 (m, 2H), 4.94 (td, J=6.1, 12.0 Hz, 1H), 4.85-4.74 (m, 1H), 4.51 (br d, J=13.0 Hz, 1H), 4.29 (br d, J=12.6 Hz, 1H), 3.85 (dd, J=4.9, 12.0 Hz, 1H), 3.31-3.10 (m, 3H), 2.97 (br s, 2H), 2.87-2.71 (m, 2H), 2.71-2.60 (m, 1H), 2.34-1.85 (m, 9H), 1.81-1.63 (m, 4H), 1.44 (d, J=6.0 Hz, 6H). LC-MS (ES⁺): m/z 778.4 [M+H]⁺.

### Example 61 Compound of Example 61 was Prepared Substantially Following the Synthesis of Example 58

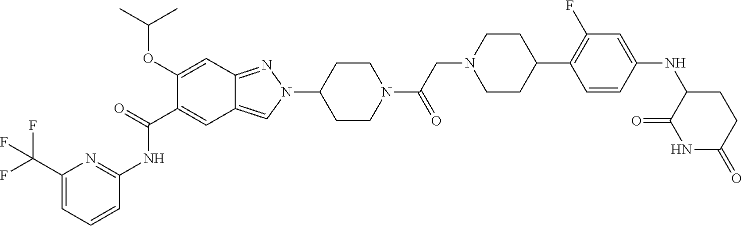 [SMILES: CC(C)Oc1cc2nn(C3CCN(C(=O)CN4CCC(c5ccc(NC6CCC(=O)NC6=O)cc5F)CC4)CC3)cc2cc1C(=O)Nc1cccc(C(F)(F)F)n1]

### 2-[1-[2-[4-[4-[(2,6-dioxo-3-piperidyl)amino]-2-fluoro-phenyl]-1-piperidyl]acetyl]-4-piperidyl]-6-isopropoxy-N-[6-(trifluoromethyl)-2-pyridyl]indazole-5-carboxamide

¹H NMR (400 MHz, DMSO-d₆) δ=11.18 (s, 1H), 10.80 (s, 1H), 9.59-9.42 (m, 1H), 8.60 (s, 1H), 8.54-8.51 (m, 2H), 8.17-8.12 (m, 1H), 7.65 (d, J=7.6 Hz, 1H), 7.23 (s, 1H), 6.98 (br s, 1H), 6.52-6.43 (m, 2H), 6.10 (br d, J=7.2 Hz, 1H), 4.95 (td, J=6.0, 12.4 Hz, 1H), 4.88-4.77 (m, 1H), 4.53 (br d, J=13.6 Hz, 1H), 4.32 (ddd, J=4.8, 7.2, 14.0 Hz, 2H), 3.93-3.47 (m, 2H), 3.18-3.08 (m, 1H), 3.03-2.86 (m, 2H), 2.79-2.53 (m, 3H), 2.44-2.35 (m, 1H), 2.28-2.15 (m, 3H), 2.14-1.74 (m, 8H), 1.45 (d, J=6.0 Hz, 6H). LC-MS (ES⁺): m/z 793.4 [M+H]⁺.

### Synthesis of N-[6-(difluoromethyl)-2-pyridyl]-6-isopropoxy-2-(4-piperidyl) indazole-5-carboxamide

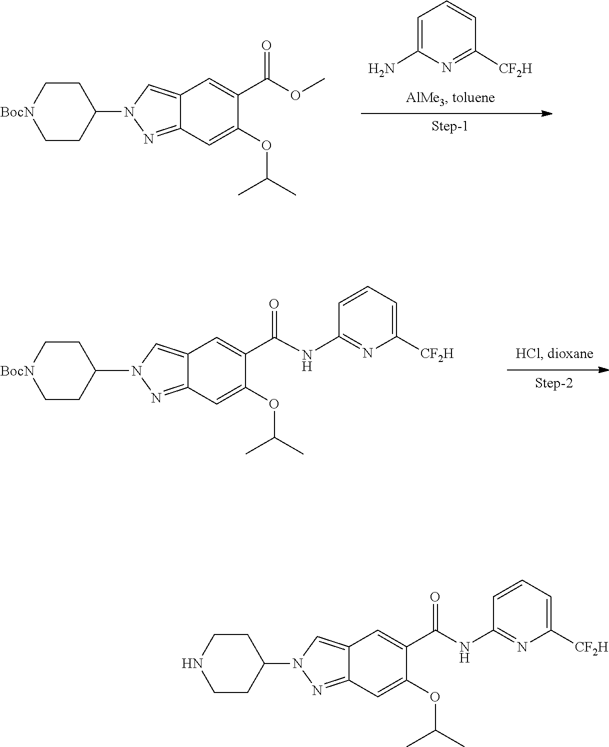 [SMILES: COC(=O)c1cc2cn(C3CCN(C(=O)OC(C)(C)C)CC3)nc2cc1OC(C)C ; Nc1cccc(C(F)F)n1 ; CC(C)Oc1cc2nn(C3CCN(C(=O)OC(C)(C)C)CC3)cc2cc1C(=O)Nc1cccc(C(F)F)n1 ; CC(C)Oc1cc2nn(C3CCNCC3)cc2cc1C(=O)Nc1cccc(C(F)F)n1]

### Step-1:

To a solution of methyl 2-(1-(tert-butoxycarbonyl)piperidin-4-yl)-6-isopropoxy-2H-indazole-5-carboxylate (840 mg, 2.01 mmol) and 6-(difluoromethyl)pyridin-2-amine (869.91 mg, 6.04 mmol) in toluene (8 mL) was added lithium bis(trimethylsilyl)azanide (1 M, 6.04 mL) at 0° C. under N₂ atmosphere. The mixture was stirred at 0° C. for 1 hour, and then warmed to 25° C. and stirred for another 15 hours. After LC-MS showed complete consumption of the reactant, the reaction mixture was diluted with H₂O (5 mL) and extracted with ethyl acetate (4 mL×3). The combined organic layers were washed with brine (5 mL), dried over anhydrous sodium sulfate, filtered, and concentrated under reduced pressure. The residue was purified by flash column chromatography (ISCO®; 20 g SepaFlash® Silica Flash Column, 0-50% ethyl acetate/petroleum ether gradient as eluent at 50 mL/min). Compound tert-butyl 4-(5-((6-(difluoromethyl)pyridin-2-yl)carbamoyl)-6-isopropoxy-2H-indazol-2-yl)piperidine-1-carboxylate (800 mg, 1.41 mmol, 70.04% yield) was obtained as a white solid. LC-MS (ES⁺): m/z 530.4 [M+H]⁺.

### Step-2:

To a stirred solution of tert-butyl 4-[5-[[6-(difluoromethyl)-2-pyridyl]carbamoyl]-6-isopropoxy-indazol-2-yl]piperidine-1-carboxylate (800 mg, 1.51 mmol) in dioxane (2 mL) was added HCl (4 M, 5.66 mL). After completion of the reaction as confirmed by LC-MS, the solvent was removed by vacuum and the crude product was triturated with ether to afford N-[6-(difluoromethyl)-2-pyridyl]-6-isopropoxy-2-(4-piperidyl) indazole-5-carboxamide HCl salt (0.74 g, 1.58 mmol, 104.59% yield) as a yellow solid. LC-MS (ES⁺): m/z 430.2[M+H]⁺.

### Example 62 Synthesis of N-[6-(difluoromethyl)-2-pyridyl]-2-[1-[2-[4-[4-(2,6-dioxo-3-piperidyl)phenyl]-1-piperidyl]acetyl]-4-piperidyl]-6-isopropoxy-indazole-5-carboxamide

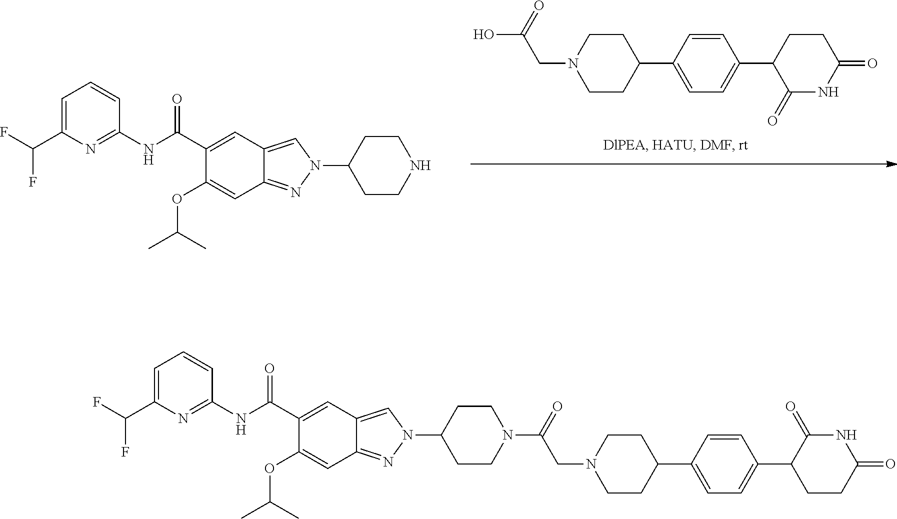 [SMILES: CC(C)Oc1cc2nn(C3CCNCC3)cc2cc1C(=O)Nc1cccc(C(F)F)n1 ; O=C(O)CN1CCC(c2ccc(C3CCC(=O)NC3=O)cc2)CC1 ; CC(C)Oc1cc2nn(C3CCN(C(=O)CN4CCC(c5ccc(C6CCC(=O)NC6=O)cc5)CC4)CC3)cc2cc1C(=O)Nc1cccc(C(F)F)n1]

To a solution of N-[6-(difluoromethyl)-2-pyridyl]-6-isopropoxy-2-(4-piperidyl) indazole-5-carboxamide (65.00 mg, 151.34 μmol) and 2-(4-(4-(2,6-dioxopiperidin-3-yl)phenyl)piperidin-1-yl)acetic acid (50 mg, 151.34 μmol) in DMF (1 mL) was added DIPEA (156.48 mg, 1.21 mmol, 210.89 μL) and HATU (86.32 mg, 227.01 μmol). The mixture was stirred at 25° C. for 16 hours. LCMS confirmed reagents were consumed completely, and one main peak with the desired mass was detected. The residue was purified by prep-HPLC (neutral condition). Compound N-(6-(difluoromethyl)pyridin-2-yl)-2-(1-(2-(4-(4-(2,6-dioxopiperidin-3-yl)phenyl)piperidin-1-yl)acetyl)piperidin-4-yl)-6-isopropoxy-2H-indazole-5-carboxamide (55.93 mg, 75.40 μmol, 49.82% yield) was obtained as a pink solid. ¹H-NMR (400 MHz, DMSO-d₆) δ ppm 11.06 (s, 1H) 10.81 (br s, 1H) 8.61 (s, 1H) 8.53 (s, 1H) 8.42 (d, J=8.4 Hz, 1H) 8.06 (t, J=8.0 Hz, 1H) 7.45 (d, J=7.6 Hz, 1H) 7.26 (s, 1H) 7.18-7.23 (m, 2H) 7.09-7.16 (m, 2H) 6.73-7.03 (m, 1H) 4.90-5.00 (m, 1H) 4.74-4.84 (m, 1H) 4.52 (br d, J=13.6 Hz, 1H) 4.30 (br d, J=13.6 Hz, 1H) 3.80 (dd, J=11.2, 4.8 Hz, 1H) 3.11-3.31 (m, 3H) 2.97 (br s, 2H) 2.83 (br t, J=12.4 Hz, 1H) 2.61-2.70 (m, 1H) 2.44-2.48 (m, 1H) 2.09-2.24 (m, 6H) 1.91-2.06 (m, 2H) 1.56-1.90 (m, 5H) 1.46 (d, J=6.0 Hz, 6H). LC-MS (ES⁺): m/z 742.4 [M+H]⁺.

### Example 63 Compound of Example 63 was Prepared Substantially Following the Synthesis of Example 62

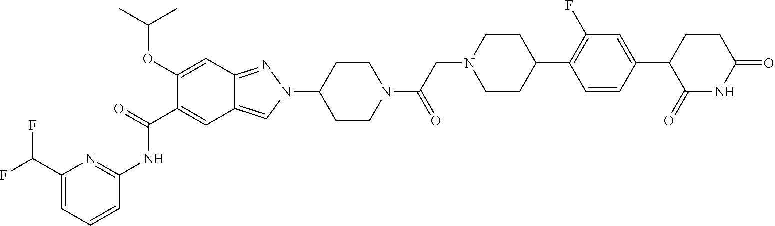 [SMILES: CC(C)Oc1cc2nn(C3CCN(C(=O)CN4CCC(c5ccc(C6CCC(=O)NC6=O)cc5F)CC4)CC3)cc2cc1C(=O)Nc1cccc(C(F)F)n1]

### N-(6-(difluoromethyl)pyridin-2-yl)-2-(1-(2-(4-(4-(2,6-dioxopiperidin-3-yl)-2-fluorophenyl)piperidin-1-yl)acetyl)piperidin-4-yl)-6-isopropoxy-2H-indazole-5-carboxamide

¹H-NMR (400 MHz, DMSO-d₆) δ ppm 11.05 (s, 1H) 10.87 (s, 1H) 9.54-9.68 (m, 1H) 8.61 (s, 1H) 8.54 (s, 1H) 8.42 (d, J=8.4 Hz, 1H) 8.06 (t, J=8.0 Hz, 1H) 7.45 (d, J=7.6 Hz, 1H) 7.26-7.33 (m, 1H) 7.23 (s, 1H) 7.08-7.15 (m, 2H) 6.74-7.03 (m, 1H) 4.91-5.00 (m, 1H) 4.80-4.91 (m, 1H) 4.54 (br d, J=13.2 Hz, 1H) 4.31-4.51 (m, 2H) 3.90 (dd, J=12.0, 4.8 Hz, 1H) 3.78-3.86 (m, 1H) 3.63 (br d, J=10.8 Hz, 2H) 3.27-3.36 (m, 2H) 3.08-3.25 (m, 3H) 2.94-3.05 (m, 1H) 2.64-2.73 (m, 1H) 2.10-2.28 (m, 6H) 1.93-2.07 (m, 4H) 1.46 (d, J=5.6 Hz, 6H). LC-MS (ES⁺): m/z 760.4 [M+H]⁺.

### Example 64 Compound of Example 64 was Prepared Substantially Following the Synthesis of Example 62

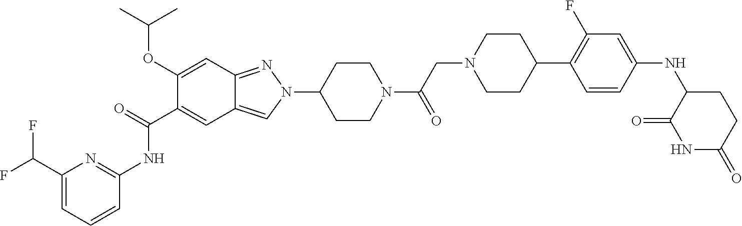 [SMILES: CC(C)Oc1cc2nn(C3CCN(C(=O)CN4CCC(c5ccc(NC6CCC(=O)NC6=O)cc5F)CC4)CC3)cc2cc1C(=O)Nc1cccc(C(F)F)n1]

### N-(6-(difluoromethyl)pyridin-2-yl)-2-(1-(2-(4-(4-((2,6-dioxopiperidin-3-yl)amino)-2-fluorophenyl)piperidin-1-yl)acetyl)piperidin-4-yl)-6-isopropoxy-2H-indazole-5-carboxamide

¹H-NMR (400 MHz, DMSO-d₆) δ ppm 11.05 (s, 1H) 10.81 (s, 1H) 9.46-9.63 (m, 1H) 8.61 (s, 1H) 8.54 (s, 1H) 8.42 (d, J=8.4 Hz, 1H) 8.06 (t, J=8.0 Hz, 1H) 7.45 (d, J=7.6 Hz, 1H) 7.23 (s, 1H) 6.73-7.03 (m, 2H) 6.46-6.55 (m, 2H) 4.92-5.02 (m, 1H) 4.80-4.91 (m, 1H) 4.54 (br d, J=14.0 Hz, 1H) 4.40-4.48 (m, 1H) 4.34 (br dd, J=12.0, 4.8 Hz, 2H) 3.98-4.03 (m, 1H) 3.82 (br d, J=12.8 Hz, 1H) 3.59 (br d, J=11.6 Hz, 1H) 3.30-3.37 (m, 1H) 3.07-3.22 (m, 2H) 2.89-3.05 (m, 2H) 2.69-2.81 (m, 1H) 2.55-2.63 (m, 1H) 2.13-2.26 (m, 3H) 1.97-2.11 (m, 4H) 1.86-1.96 (m, 3H) 1.47 (d, J=6.0 Hz, 6H). LC-MS (ES⁺): m/z 775.4 [M+H]⁺.

### Example 65 Compound of Example 65 was Prepared Substantially Following the Synthesis of Example 62

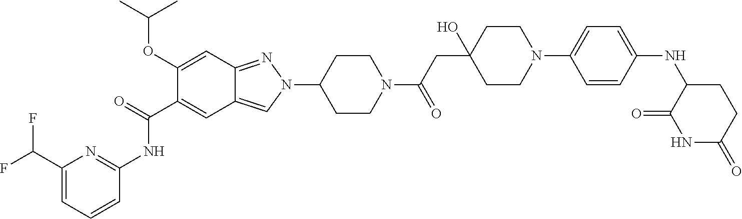 [SMILES: CC(C)Oc1cc2nn(C3CCN(C(=O)CC4(O)CCN(c5ccc(NC6CCC(=O)NC6=O)cc5)CC4)CC3)cc2cc1C(=O)Nc1cccc(C(F)F)n1]

### N-(6-(difluoromethyl)pyridin-2-yl)-2-(1-(2-(1-(4-((2,6-dioxopiperidin-3-yl)amino)phenyl)-4-hydroxypiperidin-4-yl)acetyl)piperidin-4-yl)-6-isopropoxy-2H-indazole-5-carboxamide

¹H NMR (400 MHz, DMSO-d₆) δ=11.05 (s, 1H), 10.82 (s, 1H), 8.65-8.58 (m, 1H), 8.52 (s, 1H), 8.41 (d, J=8.4 Hz, 1H), 8.05 (t, J=8.0 Hz, 1H), 7.48-7.38 (m, 2H), 7.24 (s, 1H), 7.04-6.85 (m, 1H), 6.81-6.73 (m, 2H), 6.45-6.30 (m, 1H), 5.01-4.90 (m, 1H), 4.85-4.75 (m, 1H), 4.66-4.55 (m, 1H), 4.45-4.35 (m, 1H), 4.28-4.14 (m, 1H), 3.76-3.59 (m, 2H), 3.30-3.16 (m, 2H), 2.88-2.77 (m, 1H), 2.76-2.63 (m, 3H), 2.45-2.25 (m, 2H), 2.23-2.03 (m, 6H), 2.00-1.86 (m, 4H), 1.46 (d, J=6.0 Hz, 6H). LC-MS (ES⁺): m/z 773.4 [M+H]⁺.

### Example 66 Compound of Example 66 was Prepared Substantially Following the Synthesis of Example 62

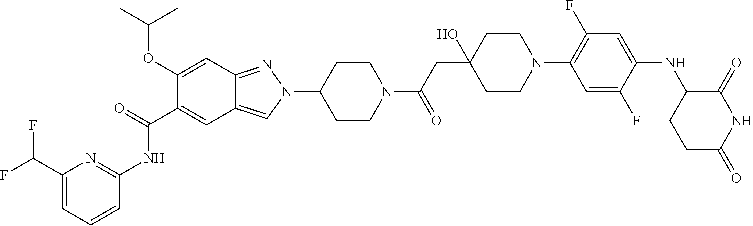 [SMILES: CC(C)Oc1cc2nn(C3CCN(C(=O)CC4(O)CCN(c5cc(F)c(NC6CCC(=O)NC6=O)cc5F)CC4)CC3)cc2cc1C(=O)Nc1cccc(C(F)F)n1]

### N-(6-(difluoromethyl)pyridin-2-yl)-2-(1-(2-(1-(4-((2,6-dioxopiperidin-3-yl)amino)-2,5-difluorophenyl)-4-hydroxypiperidin-4-yl)acetyl)piperidin-4-yl)-6-isopropoxy-2H-indazole-5-carboxamide

¹H NMR (400 MHz, DMSO-d₆) δ=11.05 (s, 1H), 10.81 (s, 1H), 8.60 (s, 1H), 8.52 (s, 1H), 8.41 (d, J=8.0 Hz, 1H), 8.05 (t, J=8.0 Hz, 1H), 7.44 (d, J=7.6 Hz, 1H), 7.26 (s, 1H), 7.04-6.70 (m, 3H), 4.95 (td, J=6.0, 12.0 Hz, 1H), 4.83-4.73 (m, 1H), 4.61 (br d, J=12.4 Hz, 1H), 4.41-4.35 (m, 1H), 4.21 (br d, J=13.2 Hz, 2H), 3.27 (br t, J=12.0 Hz, 1H), 3.04 (br d, J=0.8 Hz, 4H), 2.87-2.56 (m, 5H), 2.16 (br s, 2H), 2.08-2.00 (m, 3H), 1.94 (br dd, J=3.6, 12.0 Hz, 1H), 1.90-1.80 (m, 2H), 1.78-1.69 (m, 2H), 1.46 (d, J=6.0 Hz, 6H). LC-MS (ES⁺): m/z 809.4[M+H]⁺.

### Example 67 Compound of Example 67 was Prepared Substantially Following the Synthesis of Example 62

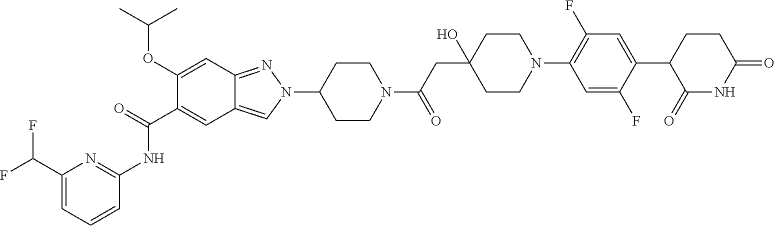 [SMILES: CC(C)Oc1cc2nn(C3CCN(C(=O)CC4(O)CCN(c5cc(F)c(C6CCC(=O)NC6=O)cc5F)CC4)CC3)cc2cc1C(=O)Nc1cccc(C(F)F)n1]

### N-(6-(difluoromethyl)pyridin-2-yl)-2-(1-(2-(1-(4-(2,6-dioxopiperidin-3-yl)-2,5-difluorophenyl)-4-hydroxypiperidin-4-yl)acetyl)piperidin-4-yl)-6-isopropoxy-2H-indazole-5-carboxamide

¹H NMR (400 MHz, DMSO-d₆) δ=11.05 (s, 1H), 10.85 (s, 1H), 8.60 (s, 1H), 8.51 (s, 1H), 8.41 (d, J=8.4 Hz, 1H), 8.05 (t, J=8.0 Hz, 1H), 7.44 (d, J=7.6 Hz, 1H), 7.26 (s, 1H), 7.11 (dd, J=6.8, 13.2 Hz, 1H), 7.03-6.73 (m, 2H), 4.95 (td, J=6.0, 12.0 Hz, 1H), 4.83-4.73 (m, 1H), 4.61 (br d, J=13.6 Hz, 1H), 4.26-4.17 (m, 1H), 4.00-3.91 (m, 2H), 3.32-3.22 (m, 1H), 3.17-3.10 (m, 2H), 3.08-2.99 (m, 2H), 2.87-2.78 (m, 1H), 2.78-2.70 (m, 1H), 2.69-2.64 (m, 1H), 2.61 (br d, J=4.4 Hz, 2H), 2.21-2.12 (m, 3H), 2.10-2.04 (m, 1H), 2.00-1.90 (m, 2H), 1.81-1.67 (m, 4H), 1.46 (d, J=6.0 Hz, 6H). LC-MS (ES⁺): m/z 784.3 [M+H]⁺.

### Synthesis of tert-butyl 4-(2-bromoacetyl)piperidine-1-carboxylate

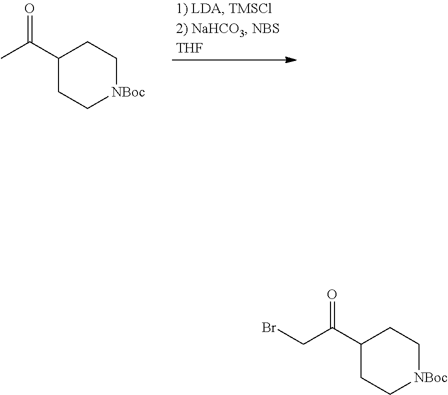 [SMILES: CC(=O)C1CCN(C(=O)OC(C)(C)C)CC1 ; CC(C)(C)OC(=O)N1CCC(C(=O)CBr)CC1]

To a solution of tert-butyl 4-acetylpiperidine-1-carboxylate (50 g, 219.97 mmol) in THE (500 mL), LDA (2 M, 131.98 mL) was added dropwise at −78° C. The solution was stirred at −78° C. for 1 hours before chlorotrimethylsilane (47.80 g, 439.95 mmol, 55.84 mL) was added at this temperature with stirring. The reaction was stirred at −78° C. for another hour. After consumption of the reactant as shown by TLC, the reaction mixture was poured into aqueous sodium bicarbonate solution (200 mL) and the aqueous phase was extracted with ethyl acetate (80 mL×3). The combined organic layers were washed with brine (50 mL), dried with anhydrous Na₂SO₄, filtered, and concentrated in vacuo. The resulting residue was then dissolved in THE (500 mL), sodium bicarbonate (27.72 g, 329.96 mmol) and N-bromosuccinimide (58.73 g, 329.96 mmol) were added at 0° C. and the solution was stirred at 25° C. for 2 hours. After TLC showed complete conversion, the reaction mixture was poured into aqueous sodium bicarbonate solution (2 L) and the aqueous phase was extracted with ethyl acetate (500 mL×3). The combined organic layers were washed with brine (500 mL), dried with anhydrous Na₂SO₄, filtered, and concentrated in vacuo. The crude product was purified by column chromatography (silica gel, petroleum ether/ethyl acetate=20/1 to 5/1). Compound tert-butyl 4-(2-bromoacetyl)piperidine-1-carboxylate (28 g, 64.31 mmol, 29.24% yield) was obtained as a yellow oil. LC-MS (ES⁺): m/z 249.9 [M-tBu+H]⁺.

### Synthesis of 5-bromo-4-isopropoxy-pyridin-2-amine

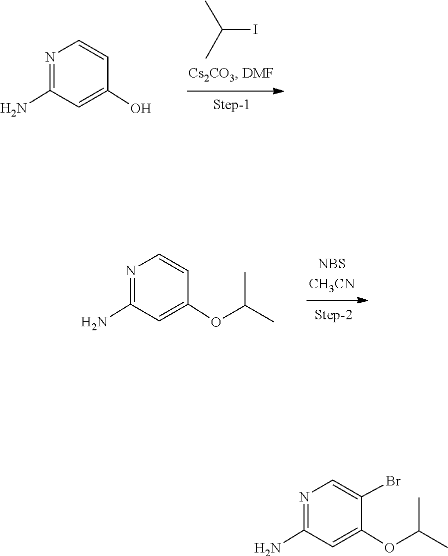

### Step-1:

To a suspension of 2-aminopyridin-4-ol (40 g, 363.26 mmol) in DMF (500 mL) was added cesium carbonate (118.36 g, 363.26 mmol) and 2-iodopropane (61.75 g, 363.26 mmol, 36.32 mL). The mixture was stirred at 120° C. for 16 hours. After consumption of the reactant as demonstrated by TLC, the reaction mixture was diluted with water (500 mL) and extracted with ethyl acetate (300 mL×4). The combined organic layers were washed with brine 300 (150 mL×2), dried over Na₂SO₄, filtered, and concentrated under reduced pressure to furnish the product 4-isopropoxypyridin-2-amine (29 g, 183.73 mmol, 50.58% yield) as a yellow solid. LC-MS (ES⁺): m/z 153.1 [M+H]⁺.

### Step-2:

To a solution of 4-isopropoxypyridin-2-amine (29 g, 190.55 mmol) in acetonitrile (300 mL) was added 1-bromopyrrolidine-2,5-dione (30.52 g, 171.49 mmol). The mixture was stirred at 25° C. for 2 hours. After consumption of the reactant as shown by TLC, the reaction mixture was diluted with water (500 mL) and extracted with ethyl acetate (200 mL×5). The combined organic layers were washed with brine (150 mL×2), dried over Na₂SO₄, filtered, and concentrated under reduced pressure. The crude product was purified by flash column chromatography (ISCO®; 250 g SepaFlash® Silica Flash Column, 10-50% ethyl acetate in pet ether as eluent at 100 mL/min). Compound 5-bromo-4-isopropoxy-pyridin-2-amine (42 g, 181.75 mmol, 95.38% yield) was obtained as an orange solid. LC-MS (ES⁺): m/z 230.9 [M+H]⁺.

### Synthesis of tert-butyl 4-[7-isopropoxy-6-(phenylcarbamoyl)imidazo[1,2-a]pyridin-2-yl]piperidine-1-carboxylate

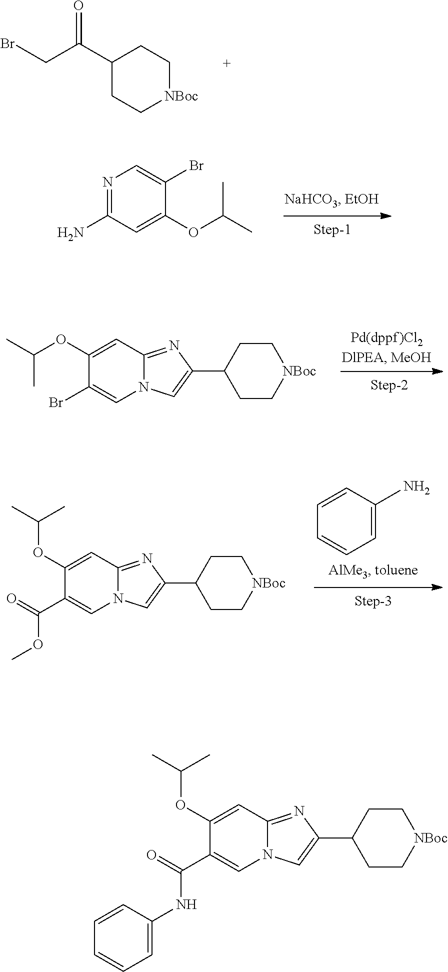 [SMILES: CC(C)(C)OC(=O)N1CCC(C(=O)CBr)CC1 ; CC(C)Oc1cc(N)ncc1Br ; CC(C)Oc1cc2nc(C3CCN(C(=O)OC(C)(C)C)CC3)cn2cc1Br ; COC(=O)c1cn2cc(C3CCN(C(=O)OC(C)(C)C)CC3)nc2cc1OC(C)C ; Nc1ccccc1 ; CC(C)Oc1cc2nc(C3CCN(C(=O)OC(C)(C)C)CC3)cn2cc1C(=O)Nc1ccccc1]

### Step-1:

A mixture of tert-butyl 4-(2-bromoacetyl)piperidine-1-carboxylate (44.06 g, 143.88 mmol), 5-bromo-4-isopropoxy-pyridin-2-amine (35 g, 151.46 mmol) and sodium bicarbonate (38.17 g, 454.37 mmol) in ethanol (800 mL) was degassed with argon three times, and the reaction mixture was stirred at 90° C. for 24 hours under argon atmosphere. After completion of the reaction, the reaction mixture was concentrated under reduced pressure to remove the solvent. The crude product was purified by flash column chromatography (ISCO®; 220 g SepaFlash® Silica Flash Column, 0-40% acetone in petroleum ether as eluent at 100 mL/min). Compound tert-butyl 4-(6-bromo-7-isopropoxy-imidazo[1,2-a]pyridin-2-yl)piperidine-1-carboxylate (35 g, 62.52 mmol, 41.28% yield) was obtained as a brown solid. LC-MS (ES⁺): m/z 440.1 [M+H]⁺.

### Step-2:

A solution of tert-butyl 4-(6-bromo-7-isopropoxy-imidazo[1,2-a]pyridin-2-yl)piperidine-1-carboxylate (13 g, 29.66 mmol), cyclopentyl(diphenyl)phosphane; dichloropalladium; iron (4.34 g, 5.93 mmol), DIPEA (45.99 g, 355.87 mmol, 61.99 mL) in methanol (450 mL) was degassed with N₂ three times, and the mixture was stirred at 80° C. for 48 hours under N₂ atmosphere. After complete consumption of the reactant, the reaction mixture was filtered and concentrated under reduced pressure. The crude product was purified by flash column chromatography (ISCO®; 80 g SepaFlash® Silica Flash Column, 0-60% ethyl acetate in petroleum ether as eluent at 60 mL/min). Compound methyl 2-(1-(tert-butoxycarbonyl)piperidin-4-yl)-7-isopropoxyimidazo[1,2-a]pyridine-6-carboxylate (12 g, 27.31 mmol, 92.07% yield) was obtained as a brown solid. LC-MS (ES⁺): m/z 418.2 [M+H]⁺.

### Step-3:

A solution of aniline (111.53 mg, 1.20 mmol, 109.34 μL) in toluene (10 mL) was added trimethylaluminum (2 M, 598.81 μL) and the mixture was degassed with N₂ three times. It was then stirred at 30° C. for 0.5 hour, and a solution of methyl 2-(1-tert-butoxycarbonyl-4-piperidyl)-7-isopropoxy-imidazo[1,2-a]pyridine-6-carboxylate (500 mg, 1.20 mmol) in toluene (10 mL) was added. The reaction mixture was stirred at 120° C. for 15.5 hours under N₂ atmosphere. After complete consumption of the reactant as shown by LC-MS, the reaction mixture was diluted with water (50 mL) and extracted with ethyl acetate (20 mL×4). The combined organic layers were washed with brine (15 mL×2), dried over Na₂SO₄, filtered, and concentrated under reduced pressure. The crude product was purified by flash column chromatography (ISCO®; 15 g SepaFlash® Silica Flash Column, 10-100% ethyl acetate in petroleum ether as eluent at 60 mL/min). Compound tert-butyl 4-[7-isopropoxy-6-(phenylcarbamoyl)imidazo[1,2-a]pyridin-2-yl]piperidine-1-carboxylate (400 mg, 819.09 μmol, 68.39% yield) was obtained as a yellow oil. LC-MS (ES⁺): m/z 479.3 [M+H]⁺.

### Tert-butyl 4-[7-isopropoxy-6-[[6-(trifluoromethyl)-2-pyridyl]carbamoyl]imidazo[1,2-a]pyridin-2-yl]piperidine-1-carboxylate

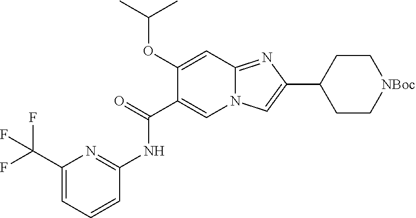 [SMILES: CC(C)Oc1cc2nc(C3CCN(C(=O)OC(C)(C)C)CC3)cn2cc1C(=O)Nc1cccc(C(F)(F)F)n1]

The synthesis was identical to that of tert-butyl 4-[7-isopropoxy-6-(phenylcarbamoyl)imidazo[1,2-a]pyridin-2-yl]piperidine-1-carboxylate, except 6-(trifluoromethyl)pyridin-2-amine was used in step-3 instead of aniline. LC-MS (ES⁺): m/z 548.2 [M+H]⁺.

### Tert-butyl 4-(6-((6-(difluoromethyl)pyridin-2-yl)carbamoyl)-7-isopropoxy imidazo[1,2-a]pyridin-2-yl)piperidine-1-carboxylate

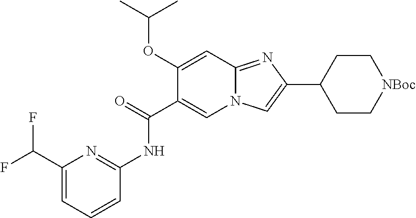 [SMILES: CC(C)Oc1cc2nc(C3CCN(C(=O)OC(C)(C)C)CC3)cn2cc1C(=O)Nc1cccc(C(F)F)n1]

The synthesis was identical to that of tert-butyl 4-[7-isopropoxy-6-(phenylcarbamoyl) imidazo[1,2-a]pyridin-2-yl]piperidine-1-carboxylate, except 6-(difluoromethyl)pyridin-2-amine was used in step-3 instead of aniline. LC-MS (ES⁺): m/z 530.3 [M+H]⁺.

### Tert-butyl 4-[7-isopropoxy-6-(2-pyridylcarbamoyl)imidazo[1,2-a]pyridin-2-yl]piperidine-1-carboxylate

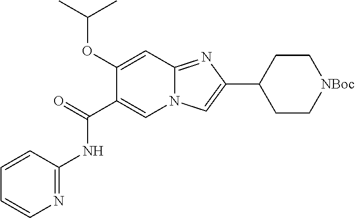 [SMILES: CC(C)Oc1cc2nc(C3CCN(C(=O)OC(C)(C)C)CC3)cn2cc1C(=O)Nc1ccccn1]

The synthesis was identical to that of tert-butyl 4-[7-isopropoxy-6-(phenylcarbamoyl) imidazo[1,2-a]pyridin-2-yl]piperidine-1-carboxylate, except pyridin-2-amine was used in step-3 instead of aniline. LC-MS (ES⁺): m/z 480.2 [M+H]⁺.

### Synthesis of tert-butyl 4-[6-[(3,4-difluorophenyl)carbamoyl]-7-isopropoxy-imidazo[1,2-a]pyridin-2-yl]piperidine-1-carboxylate

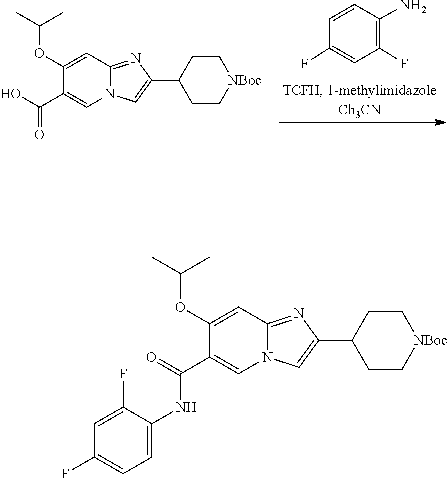 [SMILES: CC(C)Oc1cc2nc(C3CCN(C(=O)OC(C)(C)C)CC3)cn2cc1C(=O)O ; Nc1ccc(F)cc1F ; CC(C)Oc1cc2nc(C3CCN(C(=O)OC(C)(C)C)CC3)cn2cc1C(=O)Nc1ccc(F)cc1F]

To a solution of 2-(1-tert-butoxycarbonyl-4-piperidyl)-7-isopropoxy-imidazo[1,2-a]pyridine-6-carboxylic acid (160 mg, 396.56 μmol) and 2,4-difluoroaniline (51.20 mg, 396.56 μmol, 40.31 μL) in acetonitrile (2 mL) were added [chloro(dimethylamino) methylene]-dimethyl-ammonium;hexafluorophosphate (166.90 mg, 594.84 μmol) and 1-methylimidazole (97.67 mg, 1.19 mmol, 94.83 μL). The mixture was stirred at 25° C. for 10 hours. After complete consumption of the reactant as confirmed by LC-MS, the reaction mixture was concentrated under reduced pressure to remove solvent. The residue was diluted with water (10 mL) and extracted with ethyl acetate (10 mL×3). The combined organic layers were washed with aqueous NaCl (10 mL×2), dried over Na₂SO₄, filtered, and concentrated under reduced pressure. The crude product was purified by prep-TLC (pet ether/ethyl acetate=1/1) to afford compound tert-butyl 4-[6-[(2,4-difluorophenyl)carbamoyl]-7-isopropoxy-imidazo[1,2-a]pyridin-2-yl]piperidine-1-carboxylate (100 mg, 184.62 μmol, 46.56% yield) as a brown solid. LC-MS (ES⁺): m/z 515.4 [M+H]⁺.

### Synthesis of tert-butyl 4-(7-isopropoxy-6-(pyrazolo[1,5-a]pyrimidin-3-ylcarbamoyl)imidazo[1,2-a]pyridin-2-yl)piperidine-1-carboxylate

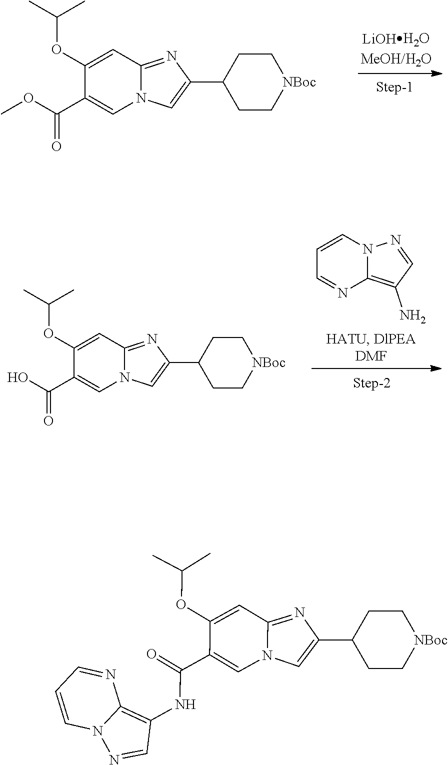 [SMILES: COC(=O)c1cn2cc(C3CCN(C(=O)OC(C)(C)C)CC3)nc2cc1OC(C)C ; CC(C)Oc1cc2nc(C3CCN(C(=O)OC(C)(C)C)CC3)cn2cc1C(=O)O ; Nc1cnn2cccnc12 ; CC(C)Oc1cc2nc(C3CCN(C(=O)OC(C)(C)C)CC3)cn2cc1C(=O)Nc1cnn2cccnc12]

### Step-1:

In a sealed tube, a solution of methyl 2-(1-tert-butoxycarbonyl-4-piperidyl)-7-isopropoxy-imidazo[1,2-a]pyridine-6-carboxylate (3.0 g, 7.19 mmol) in methanol (15 mL) was added lithium hydroxide monohydrate, 98% (753.78 mg, 17.96 mmol) and stirred for 16 hours at room temperature. Progress of the reaction was monitored by TLC and LC-MS. After completion of the reaction, the solvent was removed under reduced pressure. The residue was adjusted to pH=4 using 50% diluted HCl solution and extracted by 10% methanol in DCM. The organic layer was separated and concentrated under reduced pressure to afford 2-(1-tert-butoxycarbonyl-4-piperidyl)-7-isopropoxy-imidazo[1,2-a]pyridine-6-carboxylic acid (2.0 g, 4.56 mmol, 63.47% yield) as a gray solid. LC-MS (ES⁺): m/z 404.46 [M+H]⁺.

### Step-2:

To a solution of 2-(1-tert-butoxycarbonyl-4-piperidyl)-7-isopropoxy-imidazo[1,2-a]pyridine-6-carboxylic acid (630 mg, 1.56 mmol) and pyrazolo[1,5-a]pyrimidin-3-amine (418.90 mg, 3.12 mmol) in DMF (8 mL) was added HATU (890.57 mg, 2.34 mmol) and DIPEA (1.01 g, 7.81 mmol, 1.36 mL). The mixture was stirred at 25° C. for 20 hours while monitoring by LC-MS. Additional HATU (890.57 mg, 2.34 mmol) and pyrazolo[1,5-a]pyrimidin-3-amine (418.90 mg, 3.12 mmol) were added, and the mixture was stirred at 25° C. for another 16 hours, until LC-MS confirmed the complete consumption of the reactant. The reaction mixture was concentrated in vacuo and purified by column chromatography (silica gel, petroleum ether/ethyl acetate=20/1 to 1/1). Compound tert-butyl 4-(7-isopropoxy-6-(pyrazolo[1,5-a]pyrimidin-3-ylcarbamoyl) imidazo[1,2-a]pyridin-2-yl)piperidine-1-carboxylate (800 mg, 1.54 mmol, 98.60% yield) was obtained as a brown solid. LC-MS (ES⁺): m/z 520.3 [M+H]⁺.

### Tert-butyl 4-[7-isopropoxy-6-[(1-methylpyrazol-3-yl)carbamoyl]imidazo[1,2-a]pyridin-2-yl]piperidine-1-carboxylate

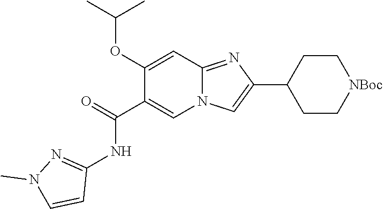 [SMILES: CC(C)Oc1cc2nc(C3CCN(C(=O)OC(C)(C)C)CC3)cn2cc1C(=O)Nc1ccn(C)n1]

Synthesis was identical to that of tert-butyl 4-(7-isopropoxy-6-(pyrazolo[1,5-a]pyrimidin-3-ylcarbamoyl)imidazo[1,2-a]pyridin-2-yl)piperidine-1-carboxylate, except 1-methylpyrazol-3-amine was used instead of pyrazolo[1,5-a]pyrimidin-3-amine in step-2. LC-MS (ES⁺): m/z 483.89 [M+H]⁺.

### Example 68 Synthesis of N-[6-(difluoromethyl)-2-pyridyl]-2-[1-[2-[4-[4-(2,6-dioxo-3-piperidyl)phenyl]-1-piperidyl]acetyl]-4-piperidyl]-7-isopropoxy-imidazo[1,2-a]pyridine-6-carboxamide

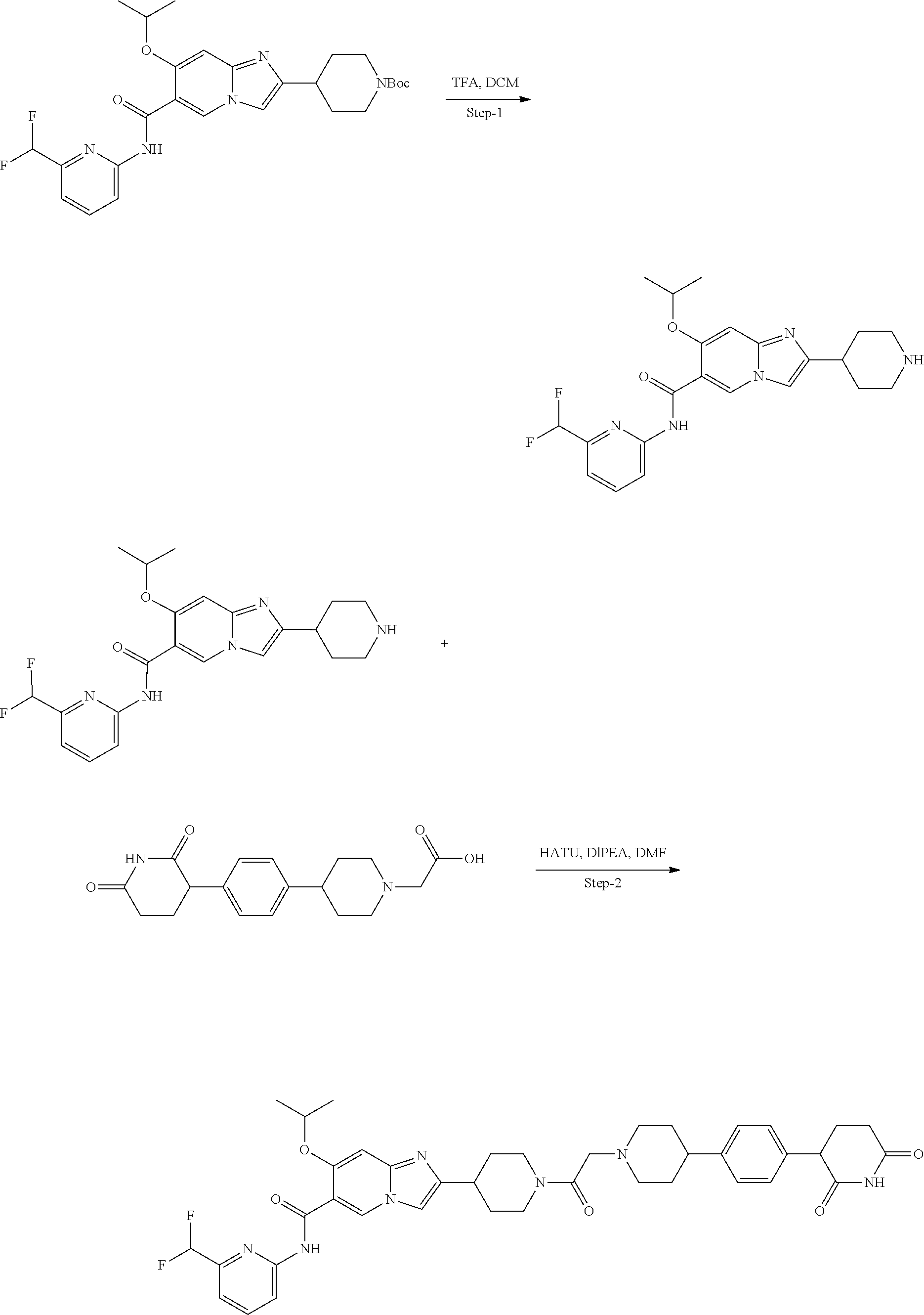 [SMILES: CC(C)Oc1cc2nc(C3CCN(C(=O)OC(C)(C)C)CC3)cn2cc1C(=O)Nc1cccc(C(F)F)n1 ; CC(C)Oc1cc2nc(C3CCNCC3)cn2cc1C(=O)Nc1cccc(C(F)F)n1 ; CC(C)Oc1cc2nc(C3CCNCC3)cn2cc1C(=O)Nc1cccc(C(F)F)n1 ; O=C(O)CN1CCC(c2ccc(C3CCC(=O)NC3=O)cc2)CC1 ; CC(C)Oc1cc2nc(C3CCN(C(=O)CN4CCC(c5ccc(C6CCC(=O)NC6=O)cc5)CC4)CC3)cn2cc1C(=O)Nc1cccc(C(F)F)n1]

### Step-1:

To a solution of tert-butyl 4-[6-[[6-(difluoromethyl)-2-pyridyl]carbamoyl]-7-isopropoxy-imidazo[1,2-a]pyridin-2-yl]piperidine-1-carboxylate (0.200 g, 377.66 μmol) in DCM (2 mL) was added TFA (430.61 mg, 3.78 mmol, 290.95 μL) at 0° C. and the reaction mixture was stirred at room temperature for 4 hours. The reaction mixture was concentrated in vacuo to yield the crude product, which was triturated with diethyl ether (5 mL) to afford N-[6-(difluoromethyl)-2-pyridyl]-7-isopropoxy-2-(4-piperidyl)imidazo[1,2-a]pyridine-6-carboxamide TFA salt (0.200 g, 364.32 μmol, 96.47% yield) as an off-white solid. LC-MS (ES⁺): m/z 430.23 [M+H]⁺.

Step-2: To a stirred solution of N-[6-(difluoromethyl)-2-pyridyl]-7-isopropoxy-2-(4-piperidyl)imidazo[1,2-a]pyridine-6-carboxamide TFA salt (0.180 g, 331.20 μmol) and 2-[4-[4-(2,6-dioxo-3-piperidyl)phenyl]-1-piperidyl]acetic acid TFA salt (147.18 mg, 331.20 μmol) in DMF (2 mL) was added DIPEA (128.41 mg, 993.59 μmol, 173.06 μL) followed by HATU (188.90 mg, 496.79 μmol). The reaction mixture was stirred for 16 hours at room temperature. Progress of the reaction was monitored by LC-MS. After consumption of the starting material, the mixture was concentrated under Genevac® to remove the solvent. The crude product was purified by prep-HPLC and lyophilized to afford N-[6-(difluoromethyl)-2-pyridyl]-2-[1-[2-[4-[4-(2,6-dioxo-3-piperidyl)phenyl]-1-piperidyl]acetyl]-4-piperidyl]-7-isopropoxy-imidazo[1,2-a]pyridine-6-carboxamide TFA salt (36.9 mg, 40.77 μmol, 12.31% yield) as an off-white solid.

¹H NMR (400 MHz, DMSO-d₆) δ 10.84 (s, 1H), 9.49 (s, 1H), 9.16 (s, 1H), 8.35 (s, 1H), 8.09 (t, J=7.9 Hz, 1H), 7.85 (s, 1H), 7.49 (d, J=7.7 Hz, 1H), 7.22 (m, 5H), 6.99 (t, J=34.8 Hz, 1H), 5.00 (d, J=5.1 Hz, 1H), 4.43 (t, J=16.4 Hz, 3H), 3.38 (m, 5H), 3.00 (m, 7H), 2.10 (m, 8H), 1.59 (d, J=5.0 Hz, 2H). 1.43 (m, J=5.0 Hz, 6H). LC-MS (ES⁺): m/z 742.18 [M+H]⁺.

### Example 69 Compound of Example 69 was Prepared Substantially Following the Synthesis of Example 68

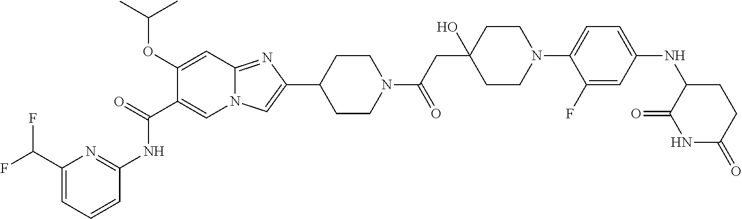 [SMILES: CC(C)Oc1cc2nc(C3CCN(C(=O)CC4(O)CCN(c5ccc(NC6CCC(=O)NC6=O)cc5F)CC4)CC3)cn2cc1C(=O)Nc1cccc(C(F)F)n1]

### N-[6-(difluoromethyl)-2-pyridyl]-2-[1-[2-[1-[4-[(2,6-dioxo-3-piperidyl)amino]-2-fluoro-phenyl]-4-hydroxy-4-piperidyl]acetyl]-4-piperidyl]-7-isopropoxy-imidazo[1,2-a]pyridine-6-carboxamide

¹H NMR (400 MHz, DMSO-d₆) δ=10.93-10.69 (m, 2H), 9.12 (s, 1H), 8.37 (br d, J=8.4 Hz, 1H), 8.08 (t, J=8.0 Hz, 1H), 7.78-7.65 (m, 1H), 7.47 (d, J=7.6 Hz, 1H), 7.19-7.10 (m, 1H), 7.05-6.73 (m, 2H), 6.50 (m, 1H), 6.44-6.38 (m, 1H), 5.77 (d, J=7.6 Hz, 1H), 5.03-4.88 (m, 2H), 4.50 (br d, J=11.2 Hz, 1H), 4.31-4.19 (m, 1H), 4.11 (m, 1H), 3.29-3.16 (m, 2H), 2.95-2.83 (m, 4H), 2.79-2.67 (m, 2H), 2.57-2.55 (m, 2H), 2.13-1.98 (m, 3H), 1.90-1.82 (m, 1H), 1.79-1.70 (m, 2H), 1.69-1.59 (m, 3H), 1.55-1.48 (m, 1H), 1.45 (d, J=6.0 Hz, 6H). LC-MS (ES⁺): m/z 791.3 [M+H]⁺.

### Example 70 Compound of Example 70 was Prepared Substantially Following the Synthesis of Example 68

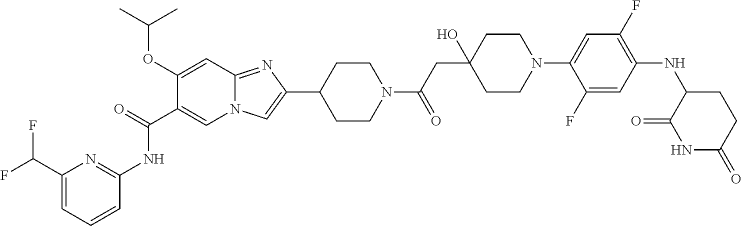 [SMILES: CC(C)Oc1cc2nc(C3CCN(C(=O)CC4(O)CCN(c5cc(F)c(NC6CCC(=O)NC6=O)cc5F)CC4)CC3)cn2cc1C(=O)Nc1cccc(C(F)F)n1]

### N-[6-(difluoromethyl)-2-pyridyl]-2-[1-[2-[1-[4-[(2,6-dioxo-3-piperidyl)amino]-2,5-difluoro-phenyl]-4-hydroxy-4-piperidyl]acetyl]-4-piperidyl]-7-isopropoxy-imidazo[1,2-a]pyridine-6-carboxamide

¹H NMR (400 MHz, DMSO-d₆) δ=11.10 (s, 1H), 10.81 (s, 1H), 9.18 (s, 1H), 8.45-8.27 (m, 1H), 8.10 (t, J=8.0 Hz, 1H), 7.99 (s, 1H), 7.51 (d, J=7.6 Hz, 1H), 7.35 (s, 1H), 7.08-6.85 (m, 2H), 6.74 (dd, J=8.4, 14.4 Hz, 1H), 5.09-4.92 (m, 1H), 4.57 (br d, J=13.2 Hz, 1H), 4.36 (br dd, J=6.0, 10.8 Hz, 1H), 4.18 (br d, J=13.6 Hz, 1H), 3.37-3.10 (m, 3H), 3.02-2.92 (m, 4H), 2.82-2.65 (m, 2H), 2.58 (s, 2H), 2.12-1.98 (m, 4H), 1.84-1.75 (m, 2H), 1.72-1.54 (m, 4H), 1.41 (d, J=3.6 Hz, 6H). LC-MS (ES⁺): m/z 809.2 [M+H]⁺.

### Example 71 Compound of Example 71 was Prepared Substantially Following the Synthesis of Example 68

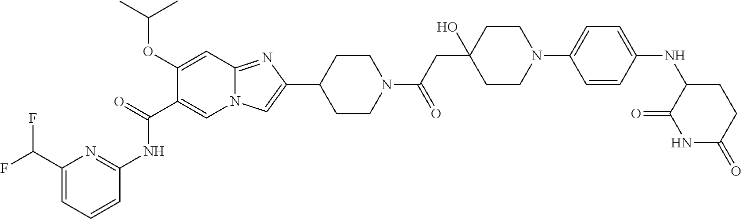 [SMILES: CC(C)Oc1cc2nc(C3CCN(C(=O)CC4(O)CCN(c5ccc(NC6CCC(=O)NC6=O)cc5)CC4)CC3)cn2cc1C(=O)Nc1cccc(C(F)F)n1]

### N-[6-(difluoromethyl)-2-pyridyl]-2-[1-[2-[1-[4-[(2,6-dioxo-3-piperidyl)amino]phenyl]-4-hydroxy-4-piperidyl]acetyl]-4-piperidyl]-7-isopropoxy-imidazo[1,2-a]pyridine-6-carboxamide

¹H NMR (400 MHz, DMSO-d₆) δ=11.07 (s, 1H), 10.83 (s, 1H), 9.18 (s, 1H), 8.35 (br s, 1H), 8.10 (t, J=8.0 Hz, 1H), 7.95 (s, 1H), 7.51 (d, J=7.6 Hz, 1H), 7.41-7.36 (m, 2H), 6.77 (br d, J=8.8 Hz, 2H), 5.03-4.93 (m, 1H), 4.56 (br d, J=12.4 Hz, 1H), 4.40 (br dd, J=4.8, 11.6 Hz, 1H), 4.16 (m, 1H), 3.36-3.07 (m, 5H), 2.93-2.57 (m, 5H), 2.16-2.04 (m, 5H), 1.97-1.86 (m, 3H), 1.67 (m, 1H), 1.61-1.50 (m, 1H), 1.42 (d, J=6.0 Hz, 6H). LC-MS (ES⁺): m/z 773.3 [M+H]⁺.

### Example 72 Compound of Example 72 was Prepared Substantially Following the Synthesis of Example 68

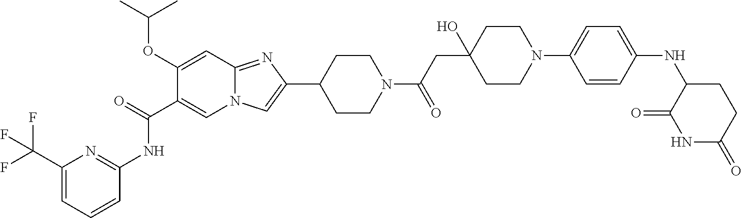 [SMILES: CC(C)Oc1cc2nc(C3CCN(C(=O)CC4(O)CCN(c5ccc(NC6CCC(=O)NC6=O)cc5)CC4)CC3)cn2cc1C(=O)Nc1cccc(C(F)(F)F)n1]

### 2-[1-[2-[1-[4-[(2,6-dioxo-3-piperidyl)amino]phenyl]-4-hydroxy-4-piperidyl]acetyl]-4-piperidyl]-7-isopropoxy-N-[6-(trifluoromethyl)-2-pyridyl]imidazo[1,2-a]pyridine-6-carboxamide

¹H NMR (400 MHz, DMSO-d₆) δ=11.21 (s, 1H), 10.83 (s, 1H), 9.18 (s, 1H), 8.58-8.35 (m, 1H), 8.19 (t, J=8.0 Hz, 1H), 7.95 (s, 1H), 7.71 (d, J=7.6 Hz, 1H), 7.45-7.23 (m, 3H), 6.77 (d, J=8.8 Hz, 2H), 5.05-4.92 (m, 1H), 4.56 (m, 1H), 4.40 (br dd, J=4.8, 12 Hz, 1H), 4.16 (br d, J=12.4 Hz, 1H), 3.61-3.56 (m, 2H), 3.25-3.13 (m, 4H), 2.83-2.56 (m, 5H), 2.22-2.03 (m, 6H), 1.92 (br dd, J=4.2, 12.4 Hz, 2H), 1.70-1.54 (m, 2H), 1.41 (br d, J=4.0 Hz, 6H). LC-MS (ES⁺): m/z 791.2 [M+H]⁺.

### Example 73 Compound of Example 73 was Prepared Substantially Following the Synthesis of Example 68

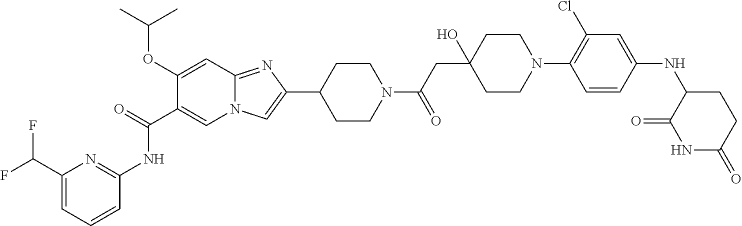 [SMILES: CC(C)Oc1cc2nc(C3CCN(C(=O)CC4(O)CCN(c5ccc(NC6CCC(=O)NC6=O)cc5Cl)CC4)CC3)cn2cc1C(=O)Nc1cccc(C(F)F)n1]

### 2-[1-[2-[1-[2-chloro-4-[(2,6-dioxo-3-piperidyl)amino]phenyl]-4-hydroxy-4-piperidyl]acetyl]-4-piperidyl]-N-[6-(difluoromethyl)-2-pyridyl]-7-isopropoxy-imidazo[1,2-a]pyridine-6-carboxamide

¹H NMR (400 MHz, DMSO-d₆) δ ppm 11.10 (s, 1H) 10.78 (s, 1H) 9.18 (s, 1H) 8.35 (s, 1H) 8.10 (t, J=7.6 Hz, 1H) 7.98 (s, 1H) 7.51 (d, J=7.6 Hz, 1H) 7.3 (s, 1H) 7.04 (br d, J=8.4 Hz, 1H) 6.77 (d, J=2.8 Hz, 1H) 6.62 (dd, J=8.8, 2.8 Hz, 1H) 4.98-5.06 (m, 1H) 4.57 (br d, J=12.8 Hz, 1H) 4.31 (dd, J=11.6, 5.2 Hz, 2H) 4.19 (br d, J=13.2 Hz, 2H) 3.16-3.26 (m, 2H) 2.85-3.03 (m, 4H) 2.69-2.79 (m, 2H) 2.59 (s, 2H) 2.03-2.12 (m, 3H) 1.76-1.91 (m, 3H) 1.63-1.74 (m, 3H) 1.56 (dd, J=12, 3.2 Hz, 1H) 1.41 (d, J=3.6 Hz, 6H). LC-MS (ES⁺): m/z 806.31 [M+H]⁺.

### Example 74 Compound of Example 74 was Prepared Substantially Following the Synthesis of Example 68

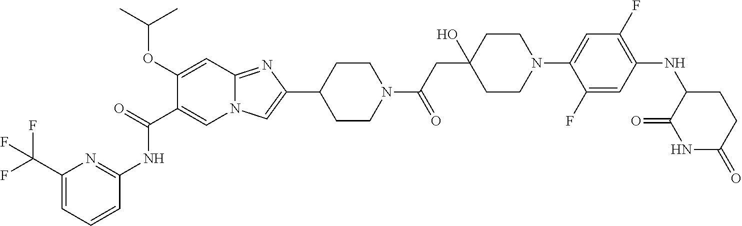 [SMILES: CC(C)Oc1cc2nc(C3CCN(C(=O)CC4(O)CCN(c5cc(F)c(NC6CCC(=O)NC6=O)cc5F)CC4)CC3)cn2cc1C(=O)Nc1cccc(C(F)(F)F)n1]

### 2-[1-[2-[1-[4-[(2,6-dioxo-3-piperidyl)amino]-2,5-difluoro-phenyl]-4-hydroxy-4-piperidyl]acetyl]-4-piperidyl]-7-isopropoxy-N-[6-(trifluoromethyl)-2-pyridyl]imidazo[1,2-a]pyridine-6-carboxamide

¹H NMR (400 MHz, DMSO-d₆) δ=10.99 (s, 1H), 10.80 (s, 1H), 9.11 (s, 1H), 8.49 (br d, J=8.4 Hz, 1H), 8.17 (t, J=8.0 Hz, 1H), 7.72 (s, 1H), 7.68 (d, J=7.6 Hz, 1H), 7.14 (s, 1H), 6.85 (dd, J=8.0, 12.8 Hz, 1H), 6.77-6.65 (m, 1H), 5.52-5.43 (m, 1H), 5.02 (s, 1H), 4.95 (m, J=1H), 4.53-4.46 (m, 1H), 4.38-4.30 (m, 1H), 4.15-4.08 (m, 1H), 2.90 (br d, J=5.2 Hz, 4H), 2.80-2.66 (m, 4H), 2.06-1.97 (m, 4H), 1.80-1.68 (m, 3H), 1.68-1.59 (m, 3H), 1.54-1.48 (m, 1H), 1.44 (d, J=6.0 Hz, 6H). LC-MS (ES⁺): m/z 827.2 [M+H]⁺.

### Example 75 Compound of Example 75 was Prepared Substantially Following the Synthesis of Example 68

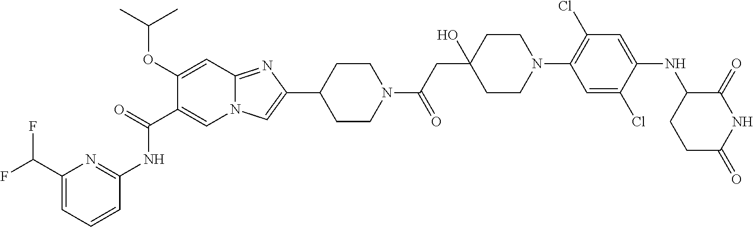 [SMILES: CC(C)Oc1cc2nc(C3CCN(C(=O)CC4(O)CCN(c5cc(Cl)c(NC6CCC(=O)NC6=O)cc5Cl)CC4)CC3)cn2cc1C(=O)Nc1cccc(C(F)F)n1]

### 2-[1-[2-[1-[2,5-dichloro-4-[(2,6-dioxo-3-piperidyl)amino]phenyl]-4-hydroxy-4-piperidyl]acetyl]-4-piperidyl]-N-[6-(difluoromethyl)-2-pyridyl]-7-isopropoxy-imidazo[1,2-a]pyridine-6-carboxamide

¹H NMR (400 MHz, DMSO-d₆) δ=10.86 (s, 1H), 9.11 (s, 1H), 8.37 (br d, J=8.3 Hz, 1H), 8.08 (t, J=8.0 Hz, 1H), 7.72 (s, 1H), 7.47 (d, J=7.7 Hz, 1H), 7.13 (d, J=6.0 Hz, 2H), 7.04-6.74 (m, 2H), 5.43 (d, J=8.0 Hz, 1H), 5.05 (s, 1H), 4.96 (td, J=6.0, 12.4 Hz, 1H), 4.54-4.43 (m, 2H), 4.17-4.07 (m, 1H), 2.93-2.79 (m, 6H), 2.10-1.98 (m, 8H), 1.80-1.60 (m, 6H), 1.45 (d, J=6.0 Hz, 6H). LC-MS (ES⁺): m/z 841.1 [M+H]⁺.

### Example 76 Compound of Example 76 was Prepared Substantially Following the Synthesis of Example 68

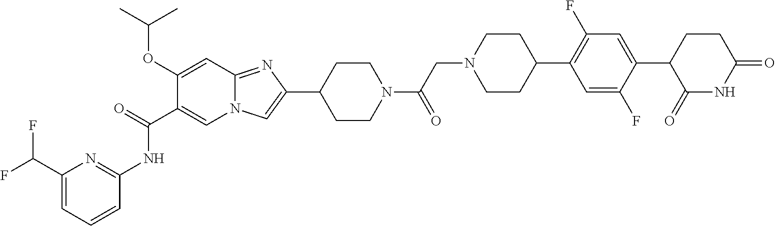 [SMILES: CC(C)Oc1cc2nc(C3CCN(C(=O)CN4CCC(c5cc(F)c(C6CCC(=O)NC6=O)cc5F)CC4)CC3)cn2cc1C(=O)Nc1cccc(C(F)F)n1]

### N-[6-(difluoromethyl)-2-pyridyl]-2-[1-[2-[4-[4-(2,6-dioxo-3-piperidyl)-2,5-difluoro-phenyl]-1-piperidyl]acetyl]-4-piperidyl]-7-isopropoxy-imidazo[1,2-a]pyridine-6-carboxamide

¹H NMR (400 MHz, DMSO-d₆) δ ppm 10.89 (br s, 1H) 10.86 (s, 1H) 9.11 (s, 1H) 8.37 (d, J=8.0 Hz, 1H) 8.08 (t, J=7.6 Hz, 1H) 7.72 (s, 1H) 7.47 (d, J=7.6 Hz, 1H) 7.14-7.21 (m, 2H) 7.13 (s, 1H) 6.73-7.04 (m, 1H) 4.90-5.02 (m, 1H) 4.40 (d, J=12.8 Hz, 1H) 4.17 (d, J=12.8 Hz, 1H) 4.03 (dd, J=12.8, 4.8 Hz, 1H) 3.26 (br s, 1H) 3.11-3.22 (m, 2H) 2.91-2.99 (m, 3H) 2.68-2.82 (m, 4H) 2.55 (d, J=3.2 Hz, 1H) 2.21 (dd, J=13.2, 3.6 Hz, 1H) 2.11-2.18 (m, 2H) 1.97-2.08 (m, 3H) 1.65-1.76 (m, 5H) 1.45 (d, J=6.0 Hz, 6H). LC-MS (ES⁺): m/z 778.4 [M+H]⁺.

### Example 77 Compound of Example 77 was Prepared Substantially Following the Synthesis of Example 68

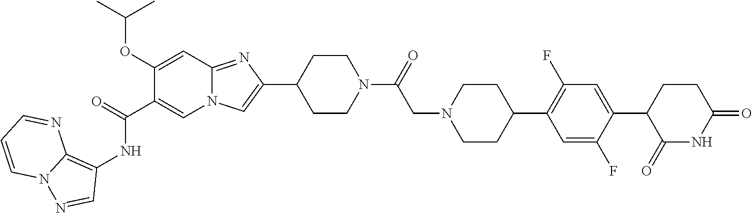 [SMILES: CC(C)Oc1cc2nc(C3CCN(C(=O)CN4CCC(c5cc(F)c(C6CCC(=O)NC6=O)cc5F)CC4)CC3)cn2cc1C(=O)Nc1cnn2cccnc12]

### 2-[1-[2-[4-[4-(2,6-dioxo-3-piperidyl)-2,5-difluoro-phenyl]-1-piperidyl]acetyl]-4-piperidyl]-7-isopropoxy-N-pyrazolo[1,5-a]pyrimidin-3-yl-imidazo[1,2-a]pyridine-6-carboxamide

¹H NMR (400 MHz, DMSO-d₆) δ ppm 10.93 (s, 1H) 10.51 (s, 1H) 9.50-9.70 (m, 1H) 9.36 (s, 1H) 9.12 (dd, J=7.2, 1.6 Hz, 1H) 8.75 (s, 1H) 8.57 (dd, J=4.0, 1.6 Hz, 1H) 8.00 (s, 1H) 7.44 (s, 1H) 7.22-7.29 (m, 1H) 7.12-7.19 (m, 1H) 7.06-7.12 (m, 1H) 5.05-5.22 (m, 1H) 4.46-4.52 (m, 1H) 4.29-4.45 (m, 2H) 4.07 (dd, J=12.8, 4.8 Hz, 1H) 3.78 (d, J=13.2 Hz, 1H) 3.27-3.35 (m, 1H) 3.08-3.26 (m, 5H) 2.90-2.98 (m, 1H) 2.70-2.81 (m, 1H) 2.56-2.60 (m, 1H) 2.21-2.30 (m, 1H) 2.07-2.18 (m, 4H) 1.96-2.06 (m, 3H) 1.66-1.91 (m, 2H) 1.57-1.65 (m, 1H) 1.55 (d, J=6.0 Hz, 6H). LC-MS (ES⁺): m/z 768.4 [M+H]⁺.

### Example 78 Compound of Example 78 was Prepared Substantially Following the Synthesis of Example 68

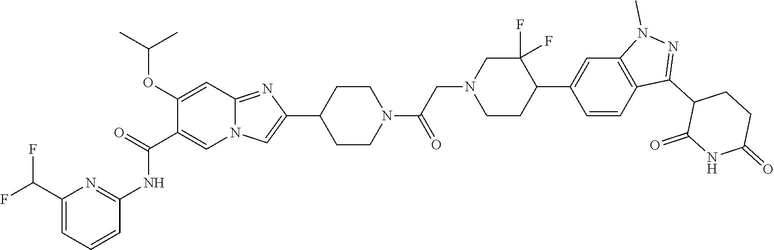 [SMILES: CC(C)Oc1cc2nc(C3CCN(C(=O)CN4CCC(c5ccc6c(C7CCC(=O)NC7=O)nn(C)c6c5)C(F)(F)C4)CC3)cn2cc1C(=O)Nc1cccc(C(F)F)n1]

### N-[6-(difluoromethyl)-2-pyridyl]-2-[1-[2-[4-[3-(2,6-dioxo-3-piperidyl)-1-methyl-indazol-6-yl]-3,3-difluoro-1-piperidyl]acetyl]-4-piperidyl]-7-isopropoxy-imidazo[1,2-a]pyridine-6-carboxamide

¹H NMR (400 MHz, DMSO-d6) δ ppm 11.09 (d, J=4.8 Hz, 1H) 10.90 (s, 1H) 9.19 (d, J=3.6 Hz, 1H) 8.29-8.42 (m, 1H) 8.10 (t, J=7.6 Hz, 1H) 7.97 (d, J=11.6 Hz, 1H) 7.68 (d, J=8.4 Hz, 1H) 7.46-7.59 (m, 2H) 7.33 (s, 1H) 7.09 (d, J=8.4 Hz, 1H) 6.73-7.06 (m, 1H) 4.96-5.08 (m, 1H) 4.48 (d, J=12.0 Hz, 1H) 4.37 (dd, J=10.0, 5.2 Hz, 1H) 4.02-4.08 (m, 1H) 4.00 (d, J=1.6 Hz, 3H) 3.12-3.31 (m, 4H) 2.80-2.88 (m, 1H) 2.61-2.75 (m, 3H) 2.56-2.61 (m, 1H) 2.31-2.45 (m, 4H) 1.92-2.25 (m, 5H) 1.52-1.76 (m, 2H) 1.42 (s, 6H). LC-MS (ES⁺): m/z 832.4 [M+H]⁺.

### Example 79 Compound of Example 79 was Prepared Substantially Following the Synthesis of Example 68

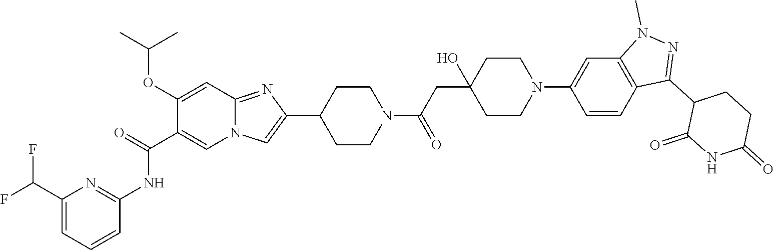 [SMILES: CC(C)Oc1cc2nc(C3CCN(C(=O)CC4(O)CCN(c5ccc6c(C7CCC(=O)NC7=O)nn(C)c6c5)CC4)CC3)cn2cc1C(=O)Nc1cccc(C(F)F)n1]

### N-[6-(difluoromethyl)-2-pyridyl]-2-[1-[2-[1-[3-(2,6-dioxo-3-piperidyl)-1-methyl-indazol-6-yl]-4-hydroxy-4-piperidyl]acetyl]-4-piperidyl]-7-isopropoxy-imidazo[1,2-a]pyridine-6-carboxamide

¹H NMR (400 MHz, DMSO-d₆) δ=11.10 (s, 1H), 10.86 (s, 1H), 9.17 (s, 1H), 8.41-8.28 (m, 1H), 8.14-8.05 (m, 1H), 7.97 (s, 1H), 7.50 (br d, J=6.8 Hz, 2H), 7.30 (s, 1H), 7.08-6.77 (m, 3H), 5.07-4.98 (m, 1H), 4.60-4.52 (m, 1H), 4.26 (br dd, J=5.2, 9.2 Hz, 1H), 4.19-4.15 (m, 1H), 3.90 (s, 3H), 3.51 (br d, J=12.0 Hz, 2H), 3.27-3.14 (m, 4H), 2.80-2.72 (m, 1H), 2.68-2.64 (m, 1H), 2.62 (br d, J=6.0 Hz, 1H), 2.59 (br s, 2H), 2.33 (br s, 1H), 2.31-2.24 (m, 1H), 2.20-2.11 (m, 1H), 2.06 (br d, J=12.0 Hz, 2H), 1.86-1.76 (m, 2H), 1.75-1.68 (m, 2H), 1.65 (br dd, J=1.6, 10.4 Hz, 1H), 1.58-1.51 (m, 1H), 1.40 (br d, J=2.0 Hz, 6H). LC-MS (ES⁺): m/z 812.4 [M+H]⁺.

### Example 80 Compound of Example 80 was Prepared Substantially Following the Synthesis of Example 68

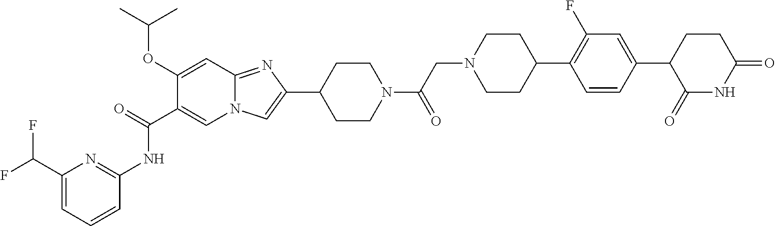 [SMILES: CC(C)Oc1cc2nc(C3CCN(C(=O)CN4CCC(c5ccc(C6CCC(=O)NC6=O)cc5F)CC4)CC3)cn2cc1C(=O)Nc1cccc(C(F)F)n1]

### N-[6-(difluoromethyl)-2-pyridyl]-2-[1-[2-[4-[4-(2,6-dioxo-3-piperidyl)-2-fluoro-phenyl]-1-piperidyl]acetyl]-4-piperidyl]-7-isopropoxy-imidazo[1,2-a]pyridine-6-carboxamide

¹H NMR (400 MHz, DMSO-d₆) δ=10.87-10.79 (m, 2H), 9.12-9.09 (m, 1H), 8.37 (d, J=8.0 Hz, 1H), 8.10-8.05 (m, 1H), 7.72-7.70 (m, 1H), 7.47 (d, J=7.2 Hz, 1H), 7.31-7.24 (m, 1H), 7.13 (s, 1H), 6.85 (s, 3H), 4.99-4.91 (m, 1H), 4.44-4.35 (m, 1H), 4.17 (br dd, J=0.8, 12.0 Hz, 1H), 3.88-3.82 (m, 1H), 3.28-3.07 (m, 3H), 3.00-2.89 (m, 3H), 2.82-2.56 (m, 5H), 2.30-2.08 (m, 3H), 2.07-1.94 (m, 3H), 1.78-1.47 (m, 5H), 1.44 (d, J=6.0 Hz, 6H). LC-MS (ES⁺): m/z 760.4. [M+H]⁺.

### Example 81 Compound of Example 81 was Prepared Substantially Following the Synthesis of Example 68

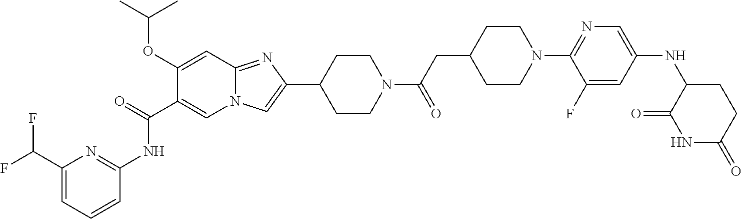 [SMILES: CC(C)Oc1cc2nc(C3CCN(C(=O)CC4CCN(c5ncc(NC6CCC(=O)NC6=O)cc5F)CC4)CC3)cn2cc1C(=O)Nc1cccc(C(F)F)n1]

### N-[6-(difluoromethyl)-2-pyridyl]-2-[1-[2-[1-[5-[(2,6-dioxo-3-piperidyl)amino]-3-fluoro-2-pyridyl]-4-piperidyl]acetyl]-4-piperidyl]-7-isopropoxy-imidazo[1,2-a]pyridine-6-carboxamide

¹H NMR (400 MHz, DMSO-d₆) δ=11.10 (s, 1H), 10.81 (s, 1H), 9.17 (s, 1H), 8.40-8.29 (m, 1H), 8.09 (t, J=8.0 Hz, 1H), 7.98 (s, 1H), 7.56 (d, J=1.6 Hz, 1H), 7.52-7.48 (m, 1H), 7.31 (s, 1H), 7.05-6.75 (m, 2H), 5.01 (b r d, J=3.6 Hz, 1H), 4.53 (br d, J=12.8 Hz, 1H), 4.30 (br d, J=4.8 Hz, 1H), 4.28 (br d, J=4.8 Hz, 2H), 3.51 (br d, J=12.4 Hz, 2H), 3.23-3.12 (m, 2H), 2.76-2.67 (m, 4H), 2.62-2.53 (m, 1H), 2.33 (br d, J=6.4 Hz, 2H), 2.12-2.00 (m, 3H), 1.92-1.82 (m, 2H), 1.75 (br d, J=12.0 Hz, 2H), 1.63-1.50 (m, 2H), 1.40 (br d, J=3.6 Hz, 6H), 1.29-1.11 (m, 1H). LC-MS (ES⁺): m/z 776.3 [M+H]⁺.

### Example 82 Compound of Example 82 was Prepared Substantially Following the Synthesis of Example 68

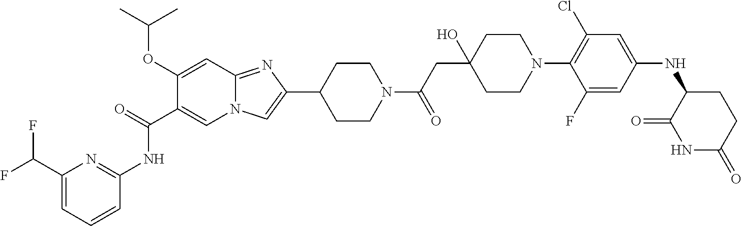 [SMILES: CC(C)Oc1cc2nc(C3CCN(C(=O)CC4(O)CCN(c5c(F)cc(N[C@H]6CCC(=O)NC6=O)cc5Cl)CC4)CC3)cn2cc1C(=O)Nc1cccc(C(F)F)n1]

### 2-[1-[2-[1-[2-chloro-4-[[(3S)-2,6-dioxo-3-piperidyl]amino]-6-fluoro-phenyl]-4-hydroxy-4-piperidyl]acetyl]-4-piperidyl]-N-[6-(difluoromethyl)-2-pyridyl]-7-isopropoxy-imidazo[1,2-a]pyridine-6-carboxamide

¹H NMR (400 MHz, DMSO-d₆) δ=10.94-10.72 (m, 2H), 9.11 (s, 1H), 8.37 (d, J=8.4 Hz, 1H), 8.07 (t, J=8.0 Hz, 1H), 7.72 (s, 1H), 7.47 (d, J=7.5 Hz, 1H), 7.14 (s, 1H), 7.05-6.73 (m, 1H), 6.58 (s, 1H), 6.45 (dd, J=2.5, 15.0 Hz, 1H), 6.22 (d, J=8.0 Hz, 1H), 5.06-4.89 (m, 2H), 4.54-4.45 (m, 1H), 4.37-4.28 (m, 1H), 4.11 (br d, J=12.8 Hz, 1H), 3.29-3.15 (m, 3H), 3.00-2.90 (m, 1H), 2.82-2.67 (m, 4H), 2.56-2.53 (m, 2H), 2.10-1.98 (m, 3H), 1.91-1.79 (m, 1H), 1.74-1.57 (m, 5H), 1.55-1.47 (m, 1H), 1.44 (d, J=6.0 Hz, 6H). LC-MS (ES⁺): m/z 825.3 [M+H]⁺.

### Example 83 Compound of Example 83 was Prepared Substantially Following the Synthesis of Example 68

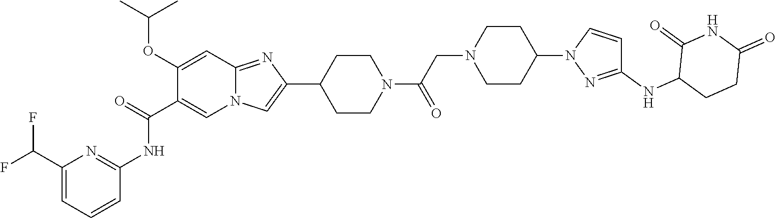 [SMILES: CC(C)Oc1cc2nc(C3CCN(C(=O)CN4CCC(n5ccc(NC6CCC(=O)NC6=O)n5)CC4)CC3)cn2cc1C(=O)Nc1cccc(C(F)F)n1]

### N-[6-(difluoromethyl)-2-pyridyl]-2-[1-[2-[4-[3-[(2,6-dioxo-3-piperidyl)amino]pyrazol-1-yl]-1-piperidyl]acetyl]-4-piperidyl]-7-isopropoxy-imidazo[1,2-a]pyridine-6-carboxamide

¹H NMR (400 MHz, DMSO-d₆) δ=11.09 (s, 1H), 10.82-10.77 (m, 1H), 9.79-9.65 (m, 1H), 9.20 (s, 1H), 8.35 (br s, 1H), 8.09 (br t, J=8.0 Hz, 1H), 7.98 (s, 1H), 7.55-7.42 (m, 3H), 7.08-6.71 (m, 1H), 5.64-5.57 (m, 1H), 4.97 (br d, J=4.4 Hz, 1H), 4.52-4.32 (m, 3H), 4.21-4.14 (m, 1H), 3.88-3.73 (m, 1H), 3.61 (br d, J=8.8 Hz, 1H), 3.42 (br s, 1H), 3.16-3.00 (m, 1H), 2.96-2.86 (m, 1H), 2.74-2.60 (m, 1H), 2.31-2.06 (m, 8H), 1.94 (dt, J=4.4, 12.4 Hz, 1H), 1.77-1.65 (m, 1H), 1.63-1.51 (m, 1H), 1.41 (br s, 6H). LC-MS (ES⁺): m/z 747.3 [M+H]⁺.

### Example 84 Compound of Example 84 was Prepared Substantially Following the Synthesis of Example 68

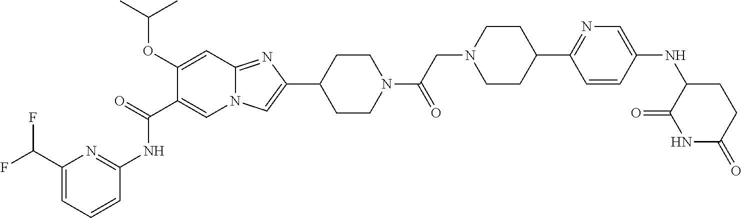 [SMILES: CC(C)Oc1cc2nc(C3CCN(C(=O)CN4CCC(c5ccc(NC6CCC(=O)NC6=O)cn5)CC4)CC3)cn2cc1C(=O)Nc1cccc(C(F)F)n1]

### N-[6-(difluoromethyl)-2-pyridyl]-2-[1-[2-[4-[5-[(2,6-dioxo-3-piperidyl)amino]-2-pyridyl]-1-piperidyl]acetyl]-4-piperidyl]-7-isopropoxy-imidazo[1,2-a]pyridine-6-carboxamide

¹H NMR (400 MHz, DMSO-d₆) δ=10.85 (s, 1H), 10.78 (s, 1H), 9.10 (s, 1H), 8.37 (br d, J=8.0 Hz, 1H), 8.10-8.04 (m, 1H), 7.96 (s, 1H), 7.72 (s, 1H), 7.47 (d, J=7.6 Hz, 1H), 7.14 (s, 1H), 7.03-6.73 (m, 4H), 5.92 (br d, J=8.0 Hz, 1H), 5.00-4.90 (m, 1H), 4.44-4.28 (m, 2H), 4.26-4.15 (m, 1H), 3.20-3.13 (m, 1H), 3.08-3.00 (m, 1H), 2.96-2.86 (m, 3H), 2.80-2.70 (m, 2H), 2.12-1.98 (m, 7H), 1.95-1.85 (m, 2H), 1.81-1.72 (m, 3H), 1.70-1.61 (m, 4H), 1.44 (d, J=6.0 Hz, 6H), 1.23 (s, 2H). LC-MS (ES⁺): m/z 758.3 [M+H]⁺.

### Example 85 Compound of Example 85 was Prepared Substantially Following the Synthesis of Example 68

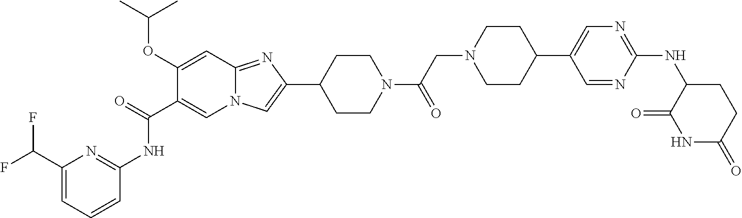 [SMILES: CC(C)Oc1cc2nc(C3CCN(C(=O)CN4CCC(c5cnc(NC6CCC(=O)NC6=O)nc5)CC4)CC3)cn2cc1C(=O)Nc1cccc(C(F)F)n1]

### N-[6-(difluoromethyl)-2-pyridyl]-2-[1-[2-[4-[2-[(2,6-dioxo-3-piperidyl)amino]pyrimidin-5-yl]-1-piperidyl]acetyl]-4-piperidyl]-7-isopropoxy-imidazo[1,2-a]pyridine-6-carboxamide

¹H NMR (400 MHz, DMSO-d₆) δ=10.86 (br s, 1H), 9.11 (s, 1H), 8.37 (d, J=8.4 Hz, 1H), 8.20 (s, 2H), 8.07 (t, J=8.0 Hz, 1H), 7.72 (s, 1H), 7.47 (d, J=7.6 Hz, 1H), 7.21-7.14 (m, 1H), 7.13 (s, 1H), 7.04-6.71 (m, 1H), 4.95 (td, J=6.0, 12.0 Hz, 1H), 4.76-4.61 (m, 1H), 4.44-4.34 (m, 1H), 4.21-4.11 (m, 1H), 3.29-3.22 (m, 2H), 3.20-3.08 (m, 2H), 2.99-2.90 (m, 2H), 2.87-2.63 (m, 1H), 2.69-2.63 (m, 1H), 2.61-2.52 (m, 2H), 2.34-2.30 (m, 1H), 2.09 (dt, J=3.6, 12.8 Hz, 3H), 2.04-1.94 (m, 2H), 1.78-1.68 (m, 2H), 1.66-1.56 (m, 2H), 1.48 (br s, 1H), 1.44 (d, J=6.0 Hz, 6H). LC-MS (ES⁺): m/z 759.3 [M+H]⁺.

### Example 86 Compound of Example 86 was Prepared Substantially Following the Synthesis of Example 68

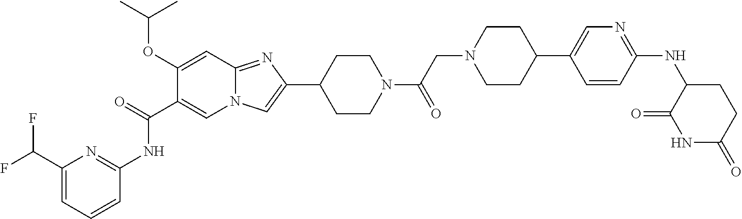 [SMILES: CC(C)Oc1cc2nc(C3CCN(C(=O)CN4CCC(c5ccc(NC6CCC(=O)NC6=O)nc5)CC4)CC3)cn2cc1C(=O)Nc1cccc(C(F)F)n1]

### N-[6-(difluoromethyl)-2-pyridyl]-2-[1-[2-[4-[6-[(2,6-dioxo-3-piperidyl)amino]-3-pyridyl]-1-piperidyl]acetyl]-4-piperidyl]-7-isopropoxy-imidazo[1,2-a]pyridine-6-carboxamide

¹H NMR (400 MHz, DMSO-d₆) δ=10.85 (s, 1H), 10.75 (s, 1H), 9.11 (s, 1H), 8.36 (br d, J=8.4 Hz, 1H), 8.07 (br t, J=7.6 Hz, 1H), 7.84 (br s, 1H), 7.72 (s, 1H), 7.47 (br d, J=7.6 Hz, 1H), 7.33 (br d, J=6.0 Hz, 1H), 7.10 (s, 1H), 6.92-6.84 (m, 1H), 6.76-6.72 (m, 1H), 6.56 (br d, J=8.8 Hz, 1H), 4.99-4.90 (m, 1H), 4.77-4.66 (m, 1H), 4.47-4.35 (m, 2H), 3.13-3.03 (m, 2H), 2.86-2.70 (m, 4H), 2.09-1.96 (m, 6H), 1.87-1.75 (m, 4H), 1.58-1.48 (m, 2H), 1.44 (br d, J=5.6 Hz, 6H), 1.30-1.19 (m, 2H), 1.05 (br t, J=6.8 Hz, 2H). LC-MS (ES⁺): m/z 758.5 [M+H]⁺.

### Example 87 Compound of Example 87 was Prepared Substantially Following the Synthesis of Example 68

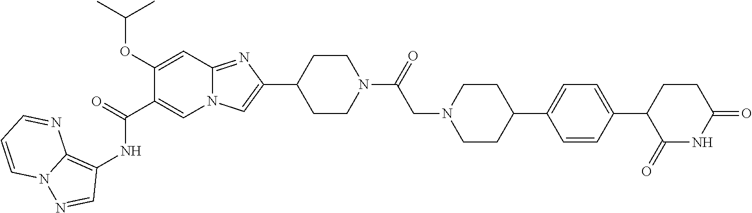 [SMILES: CC(C)Oc1cc2nc(C3CCN(C(=O)CN4CCC(c5ccc(C6CCC(=O)NC6=O)cc5)CC4)CC3)cn2cc1C(=O)Nc1cnn2cccnc12]

2-[1-[2-[4-[4-(2,6-dioxo-3-piperidyl)phenyl]-1-piperidyl]acetyl]-4-piperidyl]-7-isopropoxy-N-pyrazolo[1,5-a]pyrimidin-3-yl-imidazo[1,2-a]pyridine-6-carboxamide ¹H NMR (400 MHz, DMSO-d₆) δ 10.84 (s, 1H), 10.51 (s, 1H), 9.50 (s, 1H), 9.31 (s, 1H), 9.11 (q, J=2.8 Hz, 1H), 8.76 (s, 1H), 8.56 (q, J=1.8 Hz, 1H), 7.92 (s, 1H), 7.09 (m, J=15.4 Hz, 1H), 6.9-7.3 (m, 6H), 5.08 (m, 1H), 4.40 (m, 2H), 3.80 (m, 4H), 3.55 (d, J=31.5 Hz, 4H), 3.14 (d, J=19.0 Hz, 2H), 2.80 (m, J=16.1 Hz, 1H), 2.11 (m, 8H), 1.54 (m, 8H). LC-MS (ES⁺): m/z 732.24 [M+H]⁺. Example 88 Compound of Example 88 was Prepared Substantially Following the Synthesis of Example 68

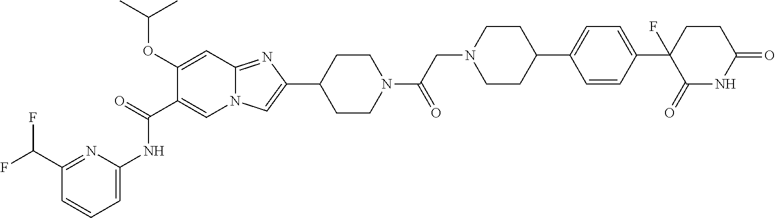 [SMILES: CC(C)Oc1cc2nc(C3CCN(C(=O)CN4CCC(c5ccc(C6(F)CCC(=O)NC6=O)cc5)CC4)CC3)cn2cc1C(=O)Nc1cccc(C(F)F)n1]

### N-[6-(difluoromethyl)-2-pyridyl]-2-[1-[2-[4-[4-(3-fluoro-2,6-dioxo-3-piperidyl) phenyl]-1-piperidyl]acetyl]-4-piperidyl]-7-isopropoxy-imidazo[1,2-a]pyridine-6-carboxamide

¹H NMR (400 MHz, DMSO-d₆) δ 10.85 (s, 1H), 9.11 (s, 1H), 8.37 (d, J=8.3 Hz, 1H), 8.07 (t, J=7.9 Hz, 1H), 7.72 (s, 1H), 7.47 (d, J=7.5 Hz, 1H), 7.35 (m, 4H), 7.04 (s, 1H), 6.89 (s, 1H), 4.95 (m, 1H), 4.40 (d, J=12.8 Hz, 1H), 4.19 (d, J=12.6 Hz, 1H), 3.21 (m, 4H), 2.91 (m, 2H), 2.66 (t, J=1.7 Hz, 2H), 2.50 (t, J=1.7 Hz, 2H), 2.28 (q, J=4.1 Hz, 1H), 2.09 (m, 4H), 1.87 (s, 2H), 1.75 (s, 2H), 1.58 (d, J=51.5 Hz, 3H), 1.44 (m, 7H). LC-MS (ES⁺): m/z 760.26 [M+H]⁺.

### Example 89 Compound of Example 89 was Prepared Substantially Following the Synthesis of Example 68

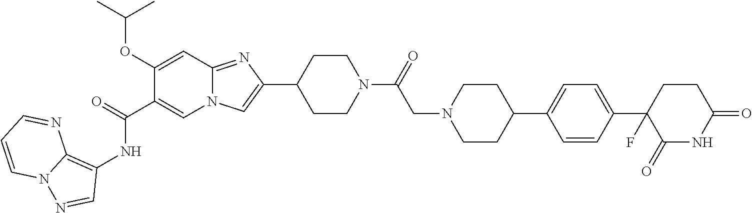 [SMILES: CC(C)Oc1cc2nc(C3CCN(C(=O)CN4CCC(c5ccc(C6(F)CCC(=O)NC6=O)cc5)CC4)CC3)cn2cc1C(=O)Nc1cnn2cccnc12]

### 2-[1-[2-[4-[4-(3-fluoro-2,6-dioxo-3-piperidyl)phenyl]-1-piperidyl]acetyl]-4-piperidyl]-7-isopropoxy-N-pyrazolo[1,5-a]pyrimidin-3-yl-imidazo[1,2-a]pyridine-6-carboxamide

¹H NMR (401 MHz, DMSO-d₆) δ 11.39 (s, 1H), 10.51 (s, 1H), 9.50 (s, 1H), 9.25 (s, 1H), 9.10 (t, J=3.5 Hz, 1H), 8.76 (s, 1H), 8.55 (t, J=1.9 Hz, 1H), 7.5 (m, 1H), 7.40 (m, 4H), 7.23 (t, J=7.6 Hz, 1H), 7.02 (m, 1H), 5.07 (t, J=5.8 Hz, 1H), 4.45 (d, J=12.8 Hz, 3H), 3.87 (m, 1H), 3.01 (m, 6H), 2.69 (d, J=17.1 Hz, 1H), 2.33 (q, J=8.6 Hz, 1H), 2.07 (m, 5H), 1.73 (t, J=11.3 Hz, 1H), 1.55 (d, J=5.9 Hz, 7H), 1.32 (t, J=13.7 Hz, 3H), 0.88 (t, J=6.4 Hz, 1H). LC-MS (ES⁺): m/z 750.33 [M+H]⁺.

### Example 90 Synthesis of N-[6-(difluoromethyl)-2-pyridyl]-2-[1-[2-[2-[4-(2,6-dioxo-3-piperidyl)phenyl]-2-azaspiro[3.3]heptan-6-yl]acetyl]-4-piperidyl]-7-isopropoxy-imidazo[1,2-a]pyridine-6-carboxamide

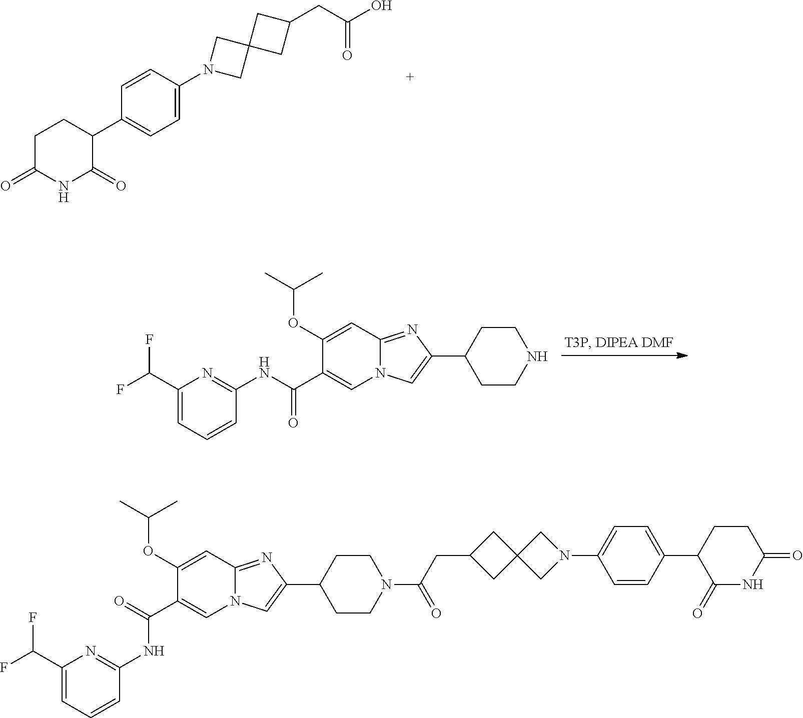 [SMILES: O=C(O)CC1CC2(C1)CN(c1ccc(C3CCC(=O)NC3=O)cc1)C2 ; CC(C)Oc1cc2nc(C3CCNCC3)cn2cc1C(=O)Nc1cccc(C(F)F)n1 ; CC(C)Oc1cc2nc(C3CCN(C(=O)CC4CC5(C4)CN(c4ccc(C6CCC(=O)NC6=O)cc4)C5)CC3)cn2cc1C(=O)Nc1cccc(C(F)F)n1]

To a stirred solution of 2-[2-[4-(2,6-dioxo-3-piperidyl)phenyl]-2-azaspiro[3.3]heptan-6-yl]acetic acid (0.12 g, 350.48 μmol) and N-(6-(difluoromethyl)pyridin-2-yl)-7-isopropoxy-2-(piperidin-4-yl)imidazo[1,2-a]pyridine-6-carboxamide TFA salt (150.52 mg, 276.95 μmol) in DMF (10 mL) were added N-ethyl-N-isopropyl-propan-2-amine (135.89 mg, 1.05 mmol, 183.14 μL). The reaction mixture was stirred at room temperature for 16 hours. After completion of the reaction as confirmed by LC-MS, the reaction mixture was concentrated under Genevac at 50° C. The crude compound was purified by prep-HPLC using the following method.

Column/dimensions: X-SELECT C18 (19*250, 5 um)

Mobile phase A: 0.05% TFA in water

Mobile phase B: 100% Acetonitrile

Gradient (Time/% B): 0/10,3/20,16.3/39.5,16.4/98,18.4/98,18.5/10,22/10

Flow rate: 17 ml/min.

Solubility: THF+WATER.

The fractions were then lyophilized to afford N-[6-(difluoromethyl)-2-pyridyl]-2-[1-[2-[2-[4-(2,6-dioxo-3-piperidyl)phenyl]-2-azaspiro[3.3]heptan-6-yl]acetyl]-4-piperidyl]-7-isopropoxy-imidazo[1,2-a]pyridine-6-carboxamide (15 mg, 19.87 umol, 5.67% yield). ¹H NMR (400 MHz, DMSO-d₆) δ 10.86 (s, 1H), 10.74 (s, 1H), 9.10 (s, 1H), 8.37 (d, J=8.2 Hz, 1H), 8.07 (t, J=8.0 Hz, 1H), 7.71 (s, 1H), 7.47 (d, J=7.5 Hz, 1H), 7.13 (s, 1H), 6.89 (m, 3H), 6.36 (d, J=8.4 Hz, 2H), 4.95 (m, 1H), 4.40 (d, J=12.5 Hz, 1H), 3.92 (d, J=12.2 Hz, 1H), 3.81 (s, 2H), 3.68 (s, 3H), 3.15 (m, 2H), 3.05 (m, 2H), 2.63 (t, J=1.7 Hz, 1H), 2.50 (t, J=1.7 Hz, 1H), 2.43 (t, J=1.7 Hz, 1H), 2.01 (m, 6H), 1.44 (m, 10H), 0.91 (s, 1H). LC-MS (ES⁺): m/z 754.16 [M+H]⁺.

### Example 91 Synthesis of (R)—N-(6-(difluoromethyl)pyridin-2-yl)-2-(1-(2-(1-(3-(2,6-dioxopiperidin-3-yl)-1-methyl-1H-indazol-6-yl)piperidin-4-yl)acetyl)piperidin-4-yl)-7-isopropoxyimidazo[1,2-a]pyridine-6-carboxamide

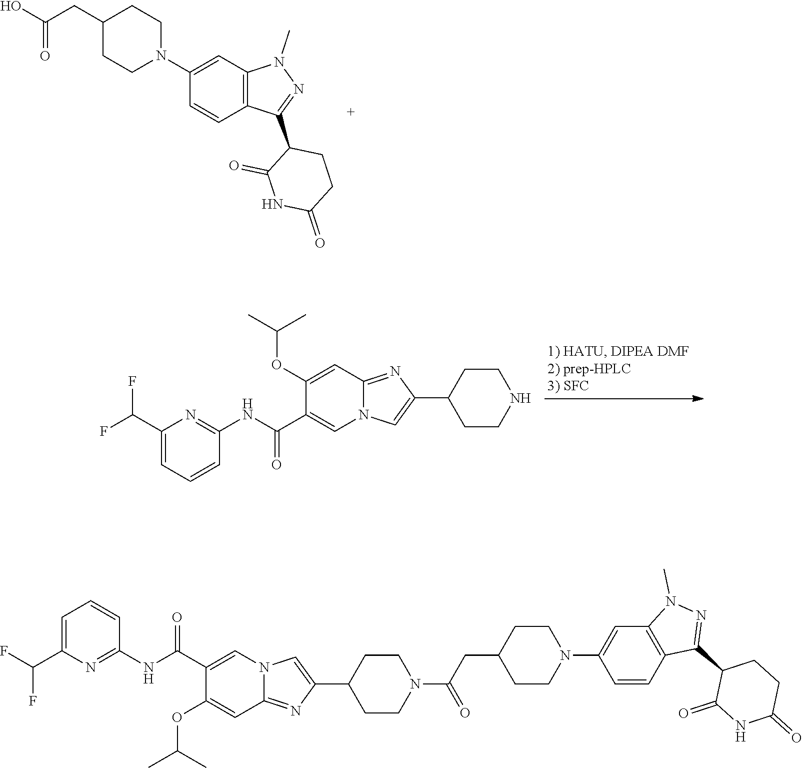 [SMILES: Cn1nc([C@H]2CCC(=O)NC2=O)c2ccc(N3CCC(CC(=O)O)CC3)cc21 ; CC(C)Oc1cc2nc(C3CCNCC3)cn2cc1C(=O)Nc1cccc(C(F)F)n1 ; CC(C)Oc1cc2nc(C3CCN(C(=O)CC4CCN(c5ccc6c([C@H]7CCC(=O)NC7=O)nn(C)c6c5)CC4)CC3)cn2cc1C(=O)Nc1cccc(C(F)F)n1]

To a solution of (R)-2-(1-(3-(2,6-dioxopiperidin-3-yl)-1-methyl-1H-indazol-6-yl)piperidin-4-yl)acetic acid (100 mg, 260.13 μmol) and N-(6-(difluoromethyl)pyridin-2-yl)-7-isopropoxy-2-(piperidin-4-yl)imidazo[1,2-a]pyridine-6-carboxamide (111.71 mg, 260.13 μmol) in DMF (2 mL) were added HATU (118.69 mg, 312.15 μmol) and DIPEA (168.10 mg, 1.30 mmol, 226.55 μL). The reaction mixture was stirred at 25° C. for 1 hour. After complete consumption of the reactant as shown by LC-MS, the reaction mixture was purified by reverse phase prep-HPLC (acetonitrile/water with TFA) to give the crude product. The crude product was further purified by SFC (Column: (S,S)Whelk-O1 100×4.6 mm I.D., 3.5 um; Mobile phase: Phase A for CO2, and Phase B for IPA+ACN(0.05% DEA);Gradient elution: 60% IPA+ACN (0.05% DEA) in CO2; Flow rate: 3 mL/min; Detector: PDA Column Temp: 35 C; Back Pressure: 100 Bar) to furnish (R)—N-(6-(difluoromethyl)pyridin-2-yl)-2-(1-(2-(1-(3-(2,6-dioxopiperidin-3-yl)-1-methyl-1H-indazol-6-yl)piperidin-4-yl)acetyl)piperidin-4-yl)-7-isopropoxyimidazo[1,2-a]pyridine-6-carboxamide (25.73 mg, 30.65 μmol, 11.78% yield) as a gray solid.

¹H NMR (400 MHz, DMSO-d₆) δ ppm 10.86 (s, 2H) 9.11 (s, 1H) 8.38 (d, J=8.4 Hz, 1H) 8.08 (br t, J=8.0 Hz, 1H) 7.72 (s, 1H) 7.48 (d, J=8.0 Hz, 2H) 7.14 (s, 1H) 6.76-7.03 (m, 3H) 4.89-5.01 (m, 1H) 4.48 (br d, J=12.8 Hz, 1H) 4.21-4.30 (m, 1H) 4.01 (m, 1H) 3.89 (s, 3H) 3.78 (br d, J=11.6 Hz, 2H) 3.30-3.32 (m, 1H) 3.16-3.22 (m, 1H) 2.91-2.97 (m, 1H) 2.71-2.77 (m, 3H) 2.60-2.63 (m, 2H) 2.31-2.36 (m, 3H) 2.14-2.18 (m, 1H) 1.99-2.07 (m, 2H) 1.88-1.95 (m, 1H) 1.80 (d, J=12.0 Hz, 2H) 1.49-1.60 (m, 2H) 1.45 (d, J=6.0 Hz, 6H) 1.34-1.38 (m, 1H). LC-MS (ES⁺): m/z 796.4 [M+H]⁺.

### Example 92 Synthesis of (S)—N-(6-(difluoromethyl)pyridin-2-yl)-2-(1-(2-(1-(3-(2,6-dioxopiperidin-3-yl)-1-methyl-1H-indazol-6-yl)piperidin-4-yl)acetyl)piperidin-4-yl)-7-isopropoxyimidazo[1,2-a]pyridine-6-carboxamide

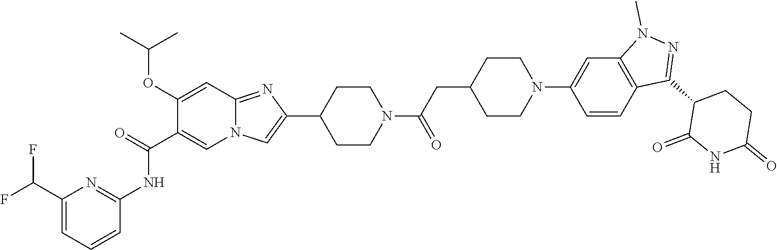 [SMILES: CC(C)Oc1cc2nc(C3CCN(C(=O)CC4CCN(c5ccc6c([C@@H]7CCC(=O)NC7=O)nn(C)c6c5)CC4)CC3)cn2cc1C(=O)Nc1cccc(C(F)F)n1]

To a solution of (S)-2-(1-(3-(2,6-dioxopiperidin-3-yl)-1-methyl-1H-indazol-6-yl)piperidin-4-yl)acetic acid (70 mg, 182.09 μmol) in DMF (2 mL) were added HATU (103.85 mg, 273.13 μmol), N-ethyl-N-isopropyl-propan-2-amine (117.67 mg, 910.44 μmol, 158.58 μL) and N-(6-(difluoromethyl)pyridin-2-yl)-7-isopropoxy-2-(piperidin-4-yl)imidazo[1,2-a]pyridine-6-carboxamide (101.66 mg, 236.72 μmol). The reaction mixture was stirred at 25° C. for 3 hours.

After completion of the reaction as confirmed by LC-MS, the reaction mixture was purified by reverse phase prep-HPLC (GX-D, Phenomenex Gemini-NX C18 75×30 mm×3 um, water (10 mM NH₄HCO₃)-ACN, Begin B:32 End B:62 Gradient Time(min):8 min) to give the crude product. The crude product was further purified by SFC (ACSWH-PREP-SFC-B, REGIS(S,S)WHELK-01(250 mm×25 mm, 10 um), IPA-CAN, Begin B:75 End B:75 Gradient Time(min):13 min) to afford (S)—N-(6-(difluoromethyl)pyridin-2-yl)-2-(1-(2-(1-(3-(2,6-dioxopiperidin-3-yl)-1-methyl-1H-indazol-6-yl)piperidin-4-yl)acetyl) piperidin-4-yl)-7-isopropoxyimidazo[1,2-a]pyridine-6-carboxamide (48.69 mg, 59.32 μmol, 32.58% yield) as a gray solid.

¹H NMR (400 MHz, DMSO-d₆) δ=10.92-10.76 (m, 2H), 9.16-9.05 (m, 1H), 8.38 (d, J=8.5 Hz, 1H), 8.08 (t, J=7.9 Hz, 1H), 7.75 (br d, J=3.3 Hz, 1H), 7.48 (d, J=8.4 Hz, 2H), 7.14 (s, 1H), 7.05-6.72 (m, 3H), 4.96 (quin, J=5.9 Hz, 1H), 4.52-4.42 (m, 1H), 4.25 (dd, J=5.1, 9.0 Hz, 1H), 4.05-3.96 (m, 1H), 3.92-3.85 (m, 3H), 3.82-3.74 (m, 2H), 3.23-3.14 (m, 1H), 2.99-2.89 (m, 1H), 2.81-2.57 (m, 6H), 2.37-2.19 (m, 4H), 2.18-2.09 (m, 1H), 2.07-1.97 (m, 2H), 1.95-1.86 (m, 1H), 1.84-1.75 (m, 2H), 1.61-1.50 (m, 1H), 1.47-1.43 (m, 6H), 1.41-1.30 (m, 2H). LC-MS (ES⁺): m/z 796.4 [M+H]⁺.

### Example 93 Synthesis of 2-[1-[2-[4-[4-[(2,6-dioxo-3-piperidyl)amino]phenyl]-1-piperidyl]-2-oxo-ethyl]-4-piperidyl]-7-isopropoxy-N-phenyl-imidazo[1,2-a]pyridine-6-carboxamide

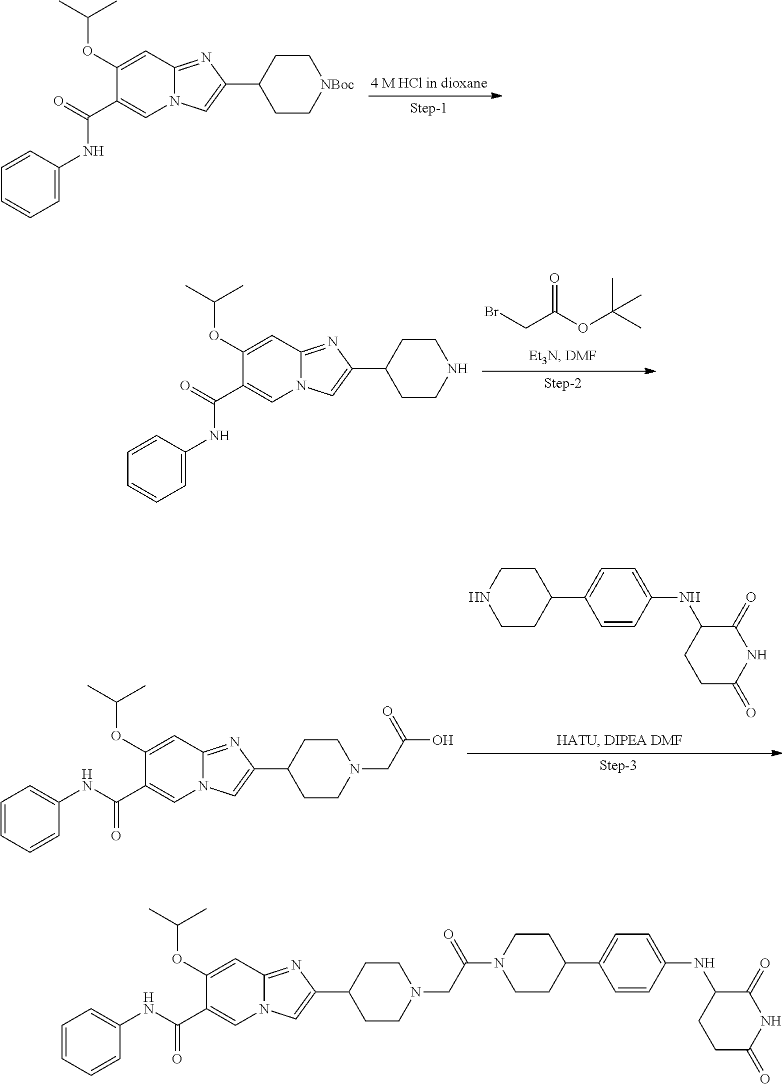

### Step-1:

To a solution of tert-butyl 4-[7-isopropoxy-6-(phenylcarbamoyl)imidazo[1,2-a]pyridin-2-yl]piperidine-1-carboxylate (400 mg, 835.80 μmol) was added HCl in dioxane (4 M, 1.04 mL) and the reaction mixture was stirred at 30° C. for 0.5 hour. After complete consumption of the reactant as shown by LC-MS, the reaction mixture was concentrated under reduced pressure to remove the solvent. The product 7-isopropoxy-N-phenyl-2-(4-piperidyl)imidazo[1,2-a]pyridine-6-carboxamide (317 mg, 816.57 μmol, 97.70% yield) was used in the next step without further purification. LC-MS (ES⁺): m/z 379.2 [M+H]⁺.

### Step-2:

To a solution of 7-isopropoxy-N-phenyl-2-(4-piperidyl)imidazo[1,2-a]pyridine-6-carboxamide (250 mg, 660.56 μmol) and tert-butyl 2-bromoacetate (154.61 mg, 792.67 μmol, 116.25 μL) in DMF (5 mL) was added triethylamine (334.21 mg, 3.30 mmol, 460.35 μL). The reaction mixture was stirred at 30° C. for 5 hours. After complete consumption of the reactant as confirmed by LC-MS, the reaction mixture was concentrated under reduced pressure to remove the solvent. The residue was diluted with water (3 mL) and extracted with ethyl acetate (15 mL×3). The combined organic layers were washed with brine (10 mL×3), dried over anhydrous sodium sulfate, filtered, and concentrated under reduced pressure. The crude product was purified by flash column chromatography (ISCO®; 12 g SepaFlash® Silica Flash Column, 50-100% ethyl acetate in petroleum ether as eluent at 60 mL/min). Compound tert-butyl 2-[4-[7-isopropoxy-6-(phenylcarbamoyl)imidazo[1,2-a]pyridin-2-yl]-1-piperidyl]acetate (110 mg, 213.21 μmol, 32.28% yield) was obtained as a white solid. LC-MS (ES⁺): m/z 493.3 [M+H]⁺.

### Step-3:

To a solution of 2-[4-[7-isopropoxy-6-(phenylcarbamoyl)imidazo[1,2-a]pyridin-2-yl]-1-piperidyl]acetic acid (70 mg, 160.37 μmol) in DMF (4 mL) was added DIPEA (207.26 mg, 1.60 mmol, 279.32 μL). HATU (91.46 mg, 240.55 μmol) was added after 5 minutes. The reaction mixture was stirred for 0.5 hour before 3-[4-(4-piperidyl)anilino]piperidine-2,6-dione (55.30 mg, 192.44 μmol) was added and the mixture was stirred at 25° C. for an additional 3 hours. After complete consumption of the reactant as shown by LC-MS, the reaction mixture was concentrated under reduced pressure to remove DMF and the residue was purified by prep-HPLC (ACSWH-GX-k/Phenomenex Gemini-NX C18 75×30 mm×3 um/water(0.225% FA)-ACN/Begin B:8-End B:38/Gradient Time(min): 7). Compound 2-[1-[2-[4-[4-[(2,6-dioxo-3-piperidyl)amino]phenyl]-1-piperidyl]-2-oxo-ethyl]-4-piperidyl]-7-isopropoxy-N-phenyl-imidazo[1,2-a]pyridine-6-carboxamide formic acid salt (41.1 mg, 54.66 μmol, 34.09% yield) was obtained as an off-white solid. ¹H NMR (400 MHz, DMSO-d₆) δ=10.76 (s, 1H), 10.11 (s, 1H), 8.91 (s, 1H), 7.69 (br d, J=7.9 Hz, 2H), 7.62 (s, 1H), 7.37 (t, J=7.9 Hz, 2H), 7.17-7.03 (m, 2H), 6.94 (d, J=8.5 Hz, 2H), 6.61 (d, J=8.5 Hz, 2H), 5.67 (br d, J=7.4 Hz, 1H), 4.84 (td, J=6.0, 12.0 Hz, 1H), 4.49 (br d, J=11.5 Hz, 1H), 4.33-4.11 (m, 3H), 3.38-3.33 (m, 1H), 3.14-2.97 (m, 3H), 2.92 (br d, J=7.6 Hz, 2H), 2.77-2.71 (m, 1H), 2.63-2.57 (m, 3H), 2.25-2.13 (m, 2H), 2.09 (td, J=4.3, 8.5 Hz, 1H), 1.98 (br t, J=13.5 Hz, 2H), 1.91-1.81 (m, 1H), 1.79-1.60 (m, 4H), 1.53 (br dd, J=2.8, 12.5 Hz, 1H), 1.40 (d, J=5.9 Hz, 6H). LC-MS (ES⁺): m/z 706.4 [M+H]⁺.

### Example 94 Compound of Example 94 was Prepared Substantially Following the Synthesis of Example 93

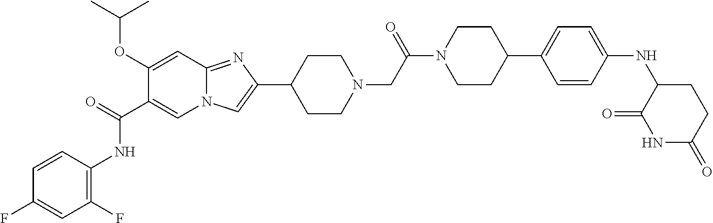 [SMILES: CC(C)Oc1cc2nc(C3CCN(CC(=O)N4CCC(c5ccc(NC6CCC(=O)NC6=O)cc5)CC4)CC3)cn2cc1C(=O)Nc1ccc(F)cc1F]

### N-(2,4-difluorophenyl)-2-[1-[2-[4-[4-[(2,6-dioxo-3-piperidyl)amino]phenyl]-1-piperidyl]-2-oxo-ethyl]-4-piperidyl]-7-isopropoxy-imidazo[1,2-a]pyridine-6-carboxamide

¹H NMR: (400 MHz, DMSO-d₆) δ=10.78 (s, 1H), 10.23-10.11 (m, 1H), 9.65-9.50 (m, 1H), 9.33-9.20 (m, 1H), 8.23-8.13 (m, 1H), 8.07-7.94 (m, 1H), 7.52-7.42 (m, 1H), 7.40-7.33 (m, 1H), 7.21-7.16 (m, 1H), 7.05-6.91 (m, 2H), 6.69-6.59 (m, 2H), 5.15-5.03 (m, 1H), 4.58-4.48 (m, 1H), 4.42-4.24 (m, 3H), 3.78-3.69 (m, 2H), 3.17-3.10 (m, 2H), 2.82-2.53 (m, 4H), 2.37-2.16 (m, 4H), 2.12-1.99 (m, 3H), 1.93-1.75 (m, 3H), 1.62-1.52 (m, 1H), 1.48-1.44 (S, 6H). LC-MS (ES⁺): m/z 742.4 [M+H]⁺.

### Example 95 Compound of Example 95 was Prepared Substantially Following the Synthesis of Example 93

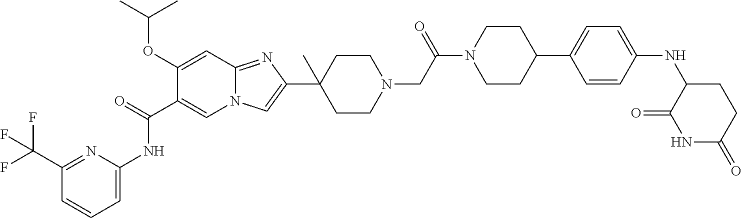 [SMILES: CC(C)Oc1cc2nc(C3(C)CCN(CC(=O)N4CCC(c5ccc(NC6CCC(=O)NC6=O)cc5)CC4)CC3)cn2cc1C(=O)Nc1cccc(C(F)(F)F)n1]

### 2-[1-[2-[4-[4-[(2,6-dioxo-3-piperidyl)amino]phenyl]-1-piperidyl]-2-oxo-ethyl]-4-methyl-4-piperidyl]-7-isopropoxy-N-[6-(trifluoromethyl)-2-pyridyl]imidazo[1,2-a]pyridine-6-carboxamide

¹H NMR (400 MHz, DMSO-d₆) δ 11.10 (s, 1H) 10.78 (s, 1H), 9.51 (s, 1H) 9.10 (s, 1H), 8.42 (s, 1H), 8.20-7.98 (m, 2H), 7.65 (s, 1H), 7.12 (s, 1H), 6.68 (t, J=28.9 Hz, 2H), 6.67 (s, 2H), 4.99 (d, J=8.4 Hz, 1H), 4.50-4.36 (m, 4H), 3.76 (m, 2H), 2.82 (m, 4H), 2.77-2.43 (m, 5H) 2.25-2.07 (m, 4H) 1.81 (m, 3H), 1.41-1.20 (m, 11H). LC-MS (ES⁺): m/z 789.40 [M+H]⁺.

### Example 96 Compound of Example 96 was Prepared Substantially Following the Synthesis of Example 93

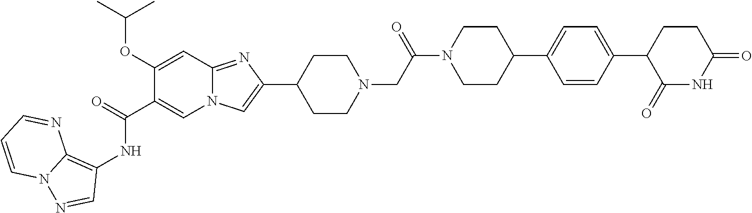 [SMILES: CC(C)Oc1cc2nc(C3CCN(CC(=O)N4CCC(c5ccc(C6CCC(=O)NC6=O)cc5)CC4)CC3)cn2cc1C(=O)Nc1cnn2cccnc12]

### 2-[1-[2-[4-[4-(2,6-dioxo-3-piperidyl)phenyl]-1-piperidyl]-2-oxo-ethyl]-4-piperidyl]-7-isopropoxy-N-pyrazolo[1,5-a]pyrimidin-3-yl-imidazo[1,2-a]pyridine-6-carboxamide

¹H NMR (400 MHz, DMSO-d₆) δ=10.83 (s, 1H), 10.52 (s, 1H), 9.77-9.56 (m, 1H), 9.42-9.25 (m, 1H), 9.12 (dd, J=1.6, 7.2 Hz, 1H), 8.76 (s, 1H), 8.57 (dd, J=1.2, 4.0 Hz, 1H), 8.04 (s, 1H), 7.46 (s, 1H), 7.25-7.15 (m, 4H), 7.10 (dd, J=4.0, 7.0 Hz, 1H), 5.17-5.06 (m, 1H), 4.55 (br d, J=12.0 Hz, 1H), 4.47-4.31 (m, 2H), 3.90-3.79 (m, 4H), 3.35-3.08 (m, 5H), 2.91-2.75 (m, 2H), 2.72-2.62 (m, 1H), 2.40-2.15 (m, 4H), 2.15-1.97 (m, 3H), 1.92-1.77 (m, 2H), 1.73-1.61 (m, 1H), 1.55 (d, J=6.0 Hz, 6H). LC-MS (ES⁺): m/z 732.2 [M+H]⁺.

### Example 97 Compound of Example 97 was Prepared Substantially Following the Synthesis of Example 93

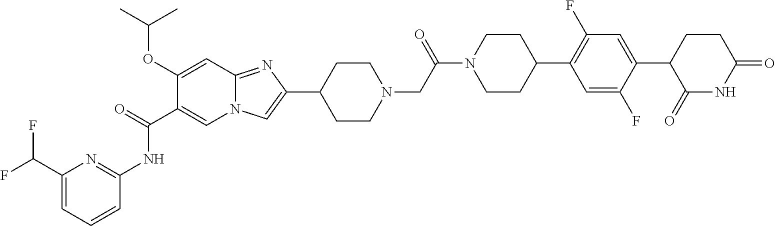 [SMILES: CC(C)Oc1cc2nc(C3CCN(CC(=O)N4CCC(c5cc(F)c(C6CCC(=O)NC6=O)cc5F)CC4)CC3)cn2cc1C(=O)Nc1cccc(C(F)F)n1]

### N-[6-(difluoromethyl)-2-pyridyl]-2-[1-[2-[4-[4-(2,6-dioxo-3-piperidyl)-2,5-difluoro-phenyl]-1-piperidyl]-2-oxo-ethyl]-4-piperidyl]-7-isopropoxy-imidazo[1,2-a]pyridine-6-carboxamide

¹H NMR (400 MHz, DMSO-d6) δ ppm 11.06 (s, 1H) 10.92 (br s, 1H) 9.59-9.79 (m, 1H) 9.11-9.25 (m, 1H) 8.25-8.43 (m, 1H) 8.10 (t, J=7.6 Hz, 1H) 8.00 (br s, 1H) 7.51 (d, J=6.8 Hz, 1H) 7.40 (s, 1H) 7.13-7.28 (m, 2H) 6.72-7.08 (m, 1H) 4.92-5.05 (m, 1H) 4.50-4.59 (m, 1H) 4.33-4.47 (m, 2H) 4.03-4.08 (m, 1H) 3.66-3.67 (m, 1H) 3.11-3.30 (m, 6H) 2.65-2.91 (m, 3H) 2.20-2.37 (m, 4H) 1.95-2.14 (m, 3H) 1.79-1.93 (m, 2H) 1.67-1.77 (m, 1H) 1.53-1.63 (m, 1H) 1.43 (br s, 6H). LC-MS (ES⁺): m/z 778.4 [M+H]⁺.

### Example 98 Compound of Example 98 was Prepared Substantially Following the Synthesis of Example 93

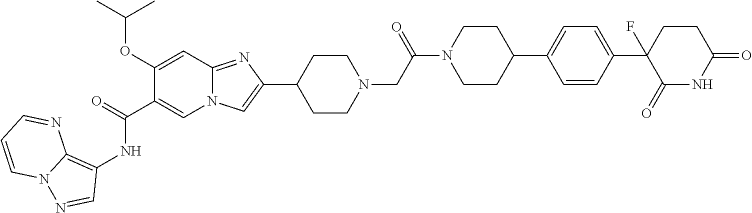 [SMILES: CC(C)Oc1cc2nc(C3CCN(CC(=O)N4CCC(c5ccc(C6(F)CCC(=O)NC6=O)cc5)CC4)CC3)cn2cc1C(=O)Nc1cnn2cccnc12]

### 2-[1-[2-[4-[4-(3-fluoro-2,6-dioxo-3-piperidyl)phenyl]-1-piperidyl]-2-oxo-ethyl]-4-piperidyl]-7-isopropoxy-N-pyrazolo[1,5-a]pyrimidin-3-yl-imidazo[1,2-a]pyridine-6-carboxamide

¹H NMR (400 MHz, DMSO-d₆) δ 11.36 (s, 1H), 10.51 (s, 1H), 9.18 (s, 1H), 9.09 (q, J=2.8 Hz, 1H), 8.77 (s, 1H), 8.54 (q, J=1.8 Hz, 1H), 8.15 (s, 1H), 7.73 (s, 1H), 7.39 (q, J=9.1 Hz, 3H), 7.19 (d, J=9.2 Hz, 2H), 7.06 (q, J=3.7 Hz, 1H), 5.04 (t, J=6.0 Hz, 1H), 4.52 (d, J=11.6 Hz, 1H), 4.26 (d, J=10.4 Hz, 1H), 3.08 (d, J=12.1 Hz, 2H), 2.87 (q, J=15.7 Hz, 4H), 2.68 (q, J=10.6 Hz, 4H), 2.18 (s, 2H), 2.03 (d, J=31.6 Hz, 2H), 1.81 (d, J=14.1 Hz, 2H), 1.66 (m, 3H), 1.54 (m, 5H), 1.45 (t, J=6.4 Hz, 1H), 1.19 (d, J=35.2 Hz, 1H), 0.85 (d, J=7.4 Hz, 1H). LC-MS (ES⁺): m/z 750.16 [M+H]⁺.

### Example 99 Compound of Example 99 was Prepared Substantially Following the Synthesis of Example 93

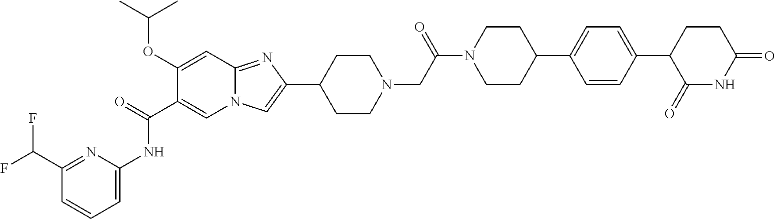 [SMILES: CC(C)Oc1cc2nc(C3CCN(CC(=O)N4CCC(c5ccc(C6CCC(=O)NC6=O)cc5)CC4)CC3)cn2cc1C(=O)Nc1cccc(C(F)F)n1]

### N-[6-(difluoromethyl)-2-pyridyl]-2-[1-[2-[4-[4-(2,6-dioxo-3-piperidyl)phenyl]-1-piperidyl]-2-oxo-ethyl]-4-piperidyl]-7-isopropoxy-imidazo[1,2-a]pyridine-6-carboxamide

¹H NMR (400 MHz, DMSO-d₆) δ 10.84 (d, J=18.4 Hz, 2H), 9.52 (s, 1H), 9.12 (s, 1H), 8.37 (d, J=8.4 Hz, 1H), 8.08 (t, J=7.9 Hz, 1H), 7.74 (s, 1H), 7.48 (d, J=7.5 Hz, 1H), 7.17 (q, J=8.1 Hz, 1H), 6.89 (t, J=54.9 Hz, 1H), 6.52 (s, 1H), 4.96 (t, J=5.8 Hz, 1H), 4.53 (d, J=12.6 Hz, 1H), 3.96 (m, 1H), 2.99 (t, J=69.4 Hz, 6H), 2.10 (m, 7H), 1.84 (m, 2H), 1.64 (m, 9H), 1.44 (m, 7H). LC-MS (ES⁻): m/z 740.25 [M−H]⁻.

### Example 100 Compound of Example 100 was Prepared Substantially Following the Synthesis of Example 93

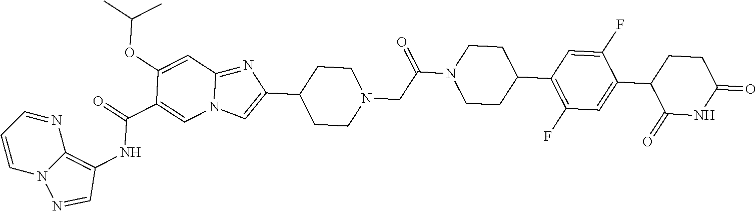 [SMILES: CC(C)Oc1cc2nc(C3CCN(CC(=O)N4CCC(c5cc(F)c(C6CCC(=O)NC6=O)cc5F)CC4)CC3)cn2cc1C(=O)Nc1cnn2cccnc12]

### 2-[1-[2-[4-[4-(2,6-dioxo-3-piperidyl)-2,5-difluoro-phenyl]-1-piperidyl]-2-oxo-ethyl]-4-piperidyl]-7-isopropoxy-N-pyrazolo[1,5-a]pyrimidin-3-yl-imidazo[1,2-a]pyridine-6-carboxamide

¹H NMR (400 MHz, DMSO-d₆) δ ppm 10.94 (s, 1H) 10.52 (s, 1H) 9.59-9.74 (m, 1H) 9.31-9.38 (m, 1H) 9.12 (dd, J=7.2, 1.6 Hz, 1H) 8.76 (s, 1H) 8.57 (dd, J=3.6, 1.2 Hz, 1H) 8.04 (s, 1H) 7.44 (s, 1H) 7.15-7.25 (m, 2H) 7.07-7.12 (m, 1H) 5.07-5.19 (m, 1H) 4.55 (d, J=12.0 Hz, 1H) 4.33-4.44 (m, 3H) 4.06 (dd, J=12.4, 4.4 Hz, 2H) 3.78 (d, J=14.8 Hz, 1H) 3.63-3.69 (m, 2H) 3.39-3.51 (m, 1H) 3.16 (d, J=3.6 Hz, 1H) 2.78-2.91 (m, 2H) 2.70-2.77 (m, 1H) 2.57 (d, J=2.8 Hz, 1H) 2.28-2.34 (m, 2H) 2.20-2.26 (m, 2H) 1.96-2.10 (m, 3H) 1.80-1.91 (m, 2H) 1.67-1.77 (m, 1H) 1.55 (d, J=6.0 Hz, 6H). LC-MS (ES⁺): m/z 768.3 [M+H]⁺.

### Example 101 Compound of Example 101 was Prepared Substantially Following the Synthesis of Example 93

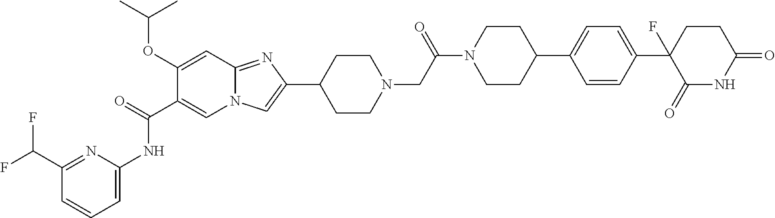 [SMILES: CC(C)Oc1cc2nc(C3CCN(CC(=O)N4CCC(c5ccc(C6(F)CCC(=O)NC6=O)cc5)CC4)CC3)cn2cc1C(=O)Nc1cccc(C(F)F)n1]

### N-[6-(difluoromethyl)-2-pyridyl]-2-[1-[2-[4-[4-(3-fluoro-2,6-dioxo-3-piperidyl)phenyl]-1-piperidyl]-2-oxo-ethyl]-4-piperidyl]-7-isopropoxy-imidazo[1,2-a]pyridine-6-carboxamide

¹H NMR (400 MHz, DMSO-d₆) δ 11.38 (s, 1H), 10.97 (s, 1H), 9.57 (s, 1H), 9.16 (d, J=9.6 Hz, 1H), 8.36 (s, 1H), 8.09 (t, J=7.9 Hz, 1H), 7.88 (s, 1H), 7.20-6.35 (m, 6H), 5.08 (m, J=14.2 Hz, 2H), 4.55 (d, J=12.1 Hz, 1H), 4.37 (q, J=16.9 Hz, 2H), 3.70 (q, J=22.1 Hz, 1H), 2.83 (m, J=12.4 Hz, 2H), 2.26 (d, J=15.6 Hz, 4H), 2.05 (t, J=12.1 Hz, 4H), 1.89 (d, J=12.8 Hz, 2H), 1.66 (d, J=9.6 Hz, 2H), 1.43-1.90 (m, 10H), 1.24 (s, 1H). LC-MS (ES⁺): m/z 760.16 [M+H]⁺.

### Example 102 Compound of Example 102 was Prepared Substantially Following the Synthesis of Example 93

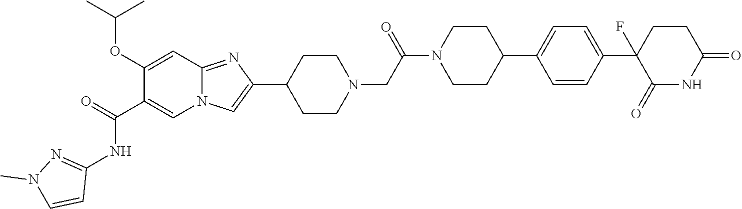 [SMILES: CC(C)Oc1cc2nc(C3CCN(CC(=O)N4CCC(c5ccc(C6(F)CCC(=O)NC6=O)cc5)CC4)CC3)cn2cc1C(=O)Nc1ccn(C)n1]

### 2-[1-[2-[4-[4-(3-fluoro-2,6-dioxo-3-piperidyl)phenyl]-1-piperidyl]-2-oxo-ethyl]-4-piperidyl]-7-isopropoxy-N-(1-methylpyrazol-3-yl)imidazo[1,2-a]pyridine-6-carboxamide

¹H NMR (400 MHz, DMSO-d₆) δ 11.38 (s, 1H), 10.58 (s, 1H), 9.62 (d, J=22.1 Hz, 1H), 9.12 (d, J=13.8 Hz, 1H), 7.94 (s, 1H), 7.65 (d, J=1.8 Hz, 1H), 6.96-7.65 (m, J=22.9 Hz, 6H), 6.58 (d, J=2.1 Hz, 1H), 4.98 (q, J=6.1 Hz, 1H), 4.54 (d, J=12.1 Hz, 1H), 4.37 (q, J=16.4 Hz, 2H), 3.78 (s, 3H), 3.11-3.78 (m, 7H), 2.83 (m, 4H), 2.49-1.90 (m, 5H), 1.66 (d, J=10.3 Hz, 1H), 1.42 (m, 7H), 1.23 (d, J=6.8 Hz, 1H). LC-MS (ES⁺): m/z 713.51 [M+H]⁺.

### Example 103 Synthesis of 2-(1-(2-(4-(4-((2,6-dioxopiperidin-3-yl)amino)phenyl)piperidin-1-yl)-2-oxoethyl)piperidin-4-yl)-7-isopropoxy-N-(6-(trifluoromethyl)pyridin-2-yl)imidazo[1,2-a]pyridine-6-carboxamide

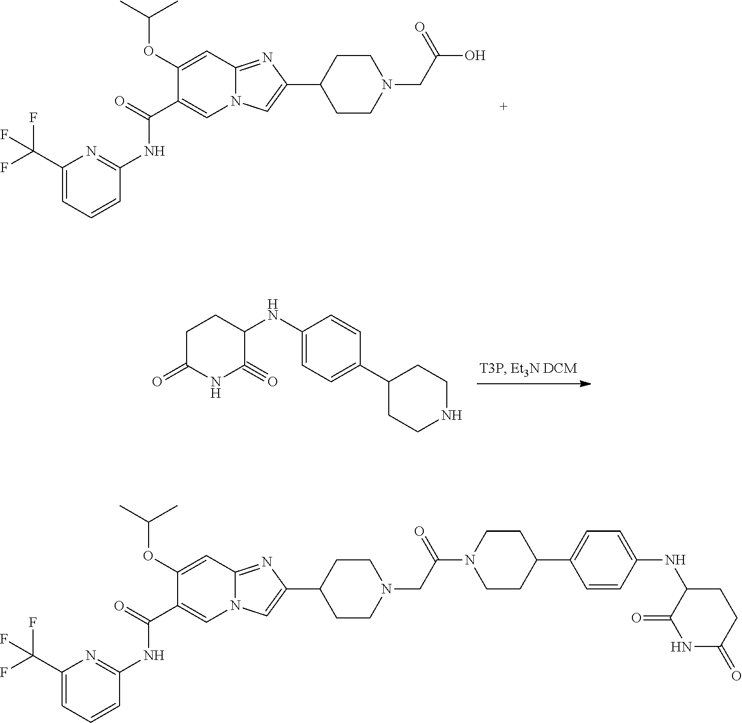

To a solution of 2-[4-[7-isopropoxy-6-[[6-(trifluoromethyl)-2-pyridyl]carbamoyl]imidazo[1,2-a]pyridin-2-yl]-1-piperidyl]acetic acid (70 mg, 138.48 μmol) in DCM (2 mL) were added N,N-diethylethanamine (56.05 mg, 553.92 μmol, 77.21 μL), 2,4,6-tripropyl-1,3,5,2,4,6-trioxatriphosphorinane-2,4,6-trioxide (110.15 mg, 207.72 μmol, 60 wt. %) and 3-((4-(piperidin-4-yl)phenyl)amino)piperidine-2,6-dione (59.69 mg, 207.72 μmol). The reaction mixture was stirred at 25° C. for 3 hours. After complete consumption of the reactant, the reaction mixture was concentrated in vacuo. The residue was purified by reverse phase prep-HPLC (ACSWH-GX-N, Phenomenex Synergi C18 150×25 mm×10 um, water (0.10% TFA)-ACN, Begin B: 20, End B: 50, Gradient Time (min): 10 min). Compound 2-(1-(2-(4-(4-((2,6-dioxopiperidin-3-yl)amino)phenyl)piperidin-1-yl)-2-oxoethyl)piperidin-4-yl)-7-isopropoxy-N-(6-(trifluoromethyl)pyridin-2-yl)imidazo[1,2-a]pyridine-6-carboxamide TFA salt (54.91 mg, 61.78 μmol, 44.61% yield) was obtained as a green solid. ¹H NMR (400 MHz, DMSO-d₆) δ=11.18 (s, 1H), 10.77 (s, 1H), 9.75-9.53 (m, 1H), 9.20-9.15 (m, 1H), 8.51-8.40 (m, 1H), 8.19 (t, J=8.1 Hz, 1H), 7.97 (s, 1H), 7.70 (d, J=7.5 Hz, 1H), 7.36 (s, 1H), 6.96 (br d, J=8.0 Hz, 2H), 6.63 (br d, J=8.4 Hz, 2H), 5.02-4.93 (m, 1H), 4.52-4.26 (m, 4H), 3.79-3.72 (m, 2H), 3.14 (br s, 2H), 2.81-2.66 (m, 4H), 2.35-2.22 (m, 3H), 2.13-2.00 (m, 4H), 1.92-1.74 (m, 4H), 1.63-1.48 (m, 2H), 1.41 (br d, J=4.8 Hz, 6H). LC-MS (ES⁺): m/z 775.2 [M+H]⁺.

### Example 104 Compound of Example 104 was Prepared Substantially Following the Synthesis of Example 103

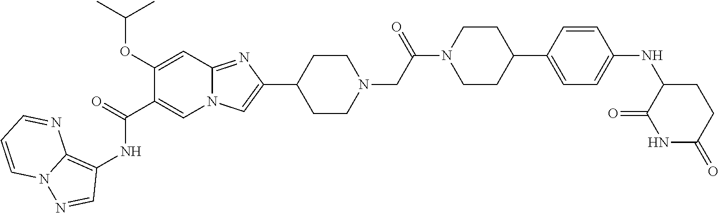 [SMILES: CC(C)Oc1cc2nc(C3CCN(CC(=O)N4CCC(c5ccc(NC6CCC(=O)NC6=O)cc5)CC4)CC3)cn2cc1C(=O)Nc1cnn2cccnc12]

### 2-[1-[2-[4-[4-[(2,6-dioxo-3-piperidyl)amino]phenyl]-1-piperidyl]-2-oxo-ethyl]-4-piperidyl]-7-isopropoxy-N-pyrazolo[1,5-a]pyrimidin-3-yl-imidazo[1,2-a]pyridine-6-carboxamide

¹H NMR (400 MHz, DMSO-d₆) δ=10.78 (s, 1H), 10.52 (s, 1H), 9.76-9.54 (m, 1H), 9.40-9.31 (m, 1H), 9.12 (dd, J=1.6, 7.2 Hz, 1H), 8.76 (s, 1H), 8.58 (dd, J=1.6, 4.0 Hz, 1H), 8.10-8.01 (m, 1H), 7.47 (s, 1H), 7.14-7.06 (m, 1H), 6.97 (br d, J=8.4 Hz, 2H), 6.64 (br d, J=8.4 Hz, 2H), 5.20-5.07 (m, 1H), 4.57-4.49 (m, 1H), 4.44-4.34 (m, 2H), 4.28 (br dd, J=4.8, 11.6 Hz, 1H), 3.38-3.27 (m, 2H), 3.20 (br d, J=12.0 Hz, 4H), 2.84-2.62 (m, 4H), 2.34-2.20 (m, 3H), 2.13-1.98 (m, 3H), 1.89-1.77 (m, 3H), 1.90 (br s, 1H), 1.55 (d, J=6.0 Hz, 6H), 1.44-1.36 (in, 1H). LC-MS (ES⁺) m/z 747.3 [M+H]⁺.

### Example 105 Compound of Example 105 was Prepared Substantially Following the Synthesis of Example 103

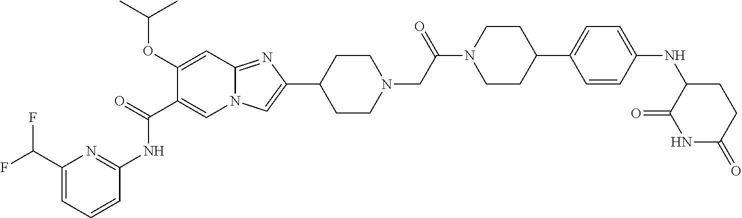 [SMILES: CC(C)Oc1cc2nc(C3CCN(CC(=O)N4CCC(c5ccc(NC6CCC(=O)NC6=O)cc5)CC4)CC3)cn2cc1C(=O)Nc1cccc(C(F)F)n1]

### N-[6-(difluoromethyl)-2-pyridyl]-2-[1-[2-[4-[4-[(2,6-dioxo-3-piperidyl)amino]phenyl]-1-piperidyl]-2-oxo-ethyl]-4-piperidyl]-7-isopropoxy-imidazo[1,2-a]pyridine-6-carboxamide

¹H NMR (400 MHz, DMSO-d₆) δ=11.11-11.00 (m, 1H), 10.81-10.72 (m, 1H), 9.75-9.55 (m, 1H), 9.25-9.11 (m, 1H), 8.44-8.30 (m, 1H), 8.10 (br t, J=8.0 Hz, 1H), 8.00-7.94 (m, 1H), 7.51 (br d, J=7.6 Hz, 1H), 7.35 (br s, 1H), 6.98-6.91 (m, 2H), 6.63 (br d, J=8.4 Hz, 2H), 5.06-4.91 (m, 1H), 4.55-4.27 (m, 4H), 3.75-3.70 (m, 2H), 3.23-3.06 (m, 5H), 2.78-2.55 (m, 4H), 2.34-2.18 (m, 3H), 2.14-1.96 (m, 3H), 1.90-1.77 (m, 3H), 1.60-1.51 (m, 1H), 1.42 (d, J=4.8 Hz, 6H). LC-MS (ES⁺): m/z 757.3 [M+H]⁺.

### Example 106 Compound of Example 106 was Prepared Substantially Following the Synthesis of Example 103

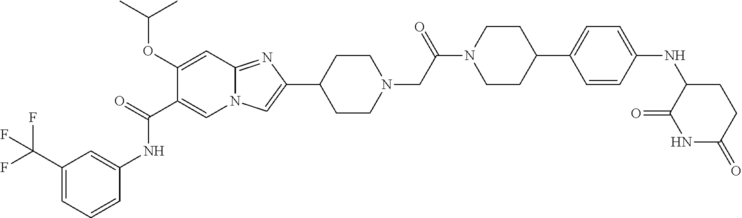 [SMILES: CC(C)Oc1cc2nc(C3CCN(CC(=O)N4CCC(c5ccc(NC6CCC(=O)NC6=O)cc5)CC4)CC3)cn2cc1C(=O)Nc1cccc(C(F)(F)F)c1]

### 2-[1-[2-[4-[4-[(2,6-dioxo-3-piperidyl)amino]phenyl]-1-piperidyl]-2-oxo-ethyl]-4-piperidyl]-7-isopropoxy-N-[3-(trifluoromethyl)phenyl]imidazo[1,2-a]pyridine-6-carboxamide

¹H NMR (400 MHz, DMSO-d₆) δ=10.78 (s, 1H), 10.66 (s, 1H), 9.70-9.54 (m, 1H), 9.13-9.07 (m, 1H), 8.21 (br s, 1H), 7.95 (br s, 1H), 7.88 (br d, J=8.4 Hz, 1H), 7.65 (br t, J=8.1 Hz, 1H), 7.55-7.50 (m, 1H), 7.38-7.32 (m, 1H), 7.00-6.92 (m, 2H), 6.64 (br d, J=8.4 Hz, 2H), 5.00-4.89 (m, 1H), 4.53-4.24 (m, 4H), 3.75-3.70 (m, 2H), 3.25-3.08 (m, 5H), 2.81-2.62 (m, 4H), 2.35-2.19 (m, 3H), 2.14-1.94 (m, 3H), 1.92-1.76 (m, 3H), 1.64-1.52 (m, 1H), 1.41 (br d, J=6.0 Hz, 6H). LC-MS (ES⁺): m/z 774.3 [M+H]⁺.

### Example 107 Compound of Example 107 was Prepared Substantially Following the Synthesis of Example 103

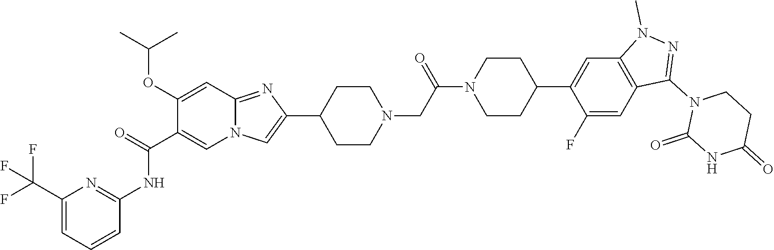 [SMILES: CC(C)Oc1cc2nc(C3CCN(CC(=O)N4CCC(c5cc6c(cc5F)c(N5CCC(=O)NC5=O)nn6C)CC4)CC3)cn2cc1C(=O)Nc1cccc(C(F)(F)F)n1]

### 2-[1-[2-[4-[3-(2,4-dioxohexahydropyrimidin-1-yl)-5-fluoro-1-methyl-indazol-6-yl]piperazin-1-yl]-2-oxo-ethyl]-4-piperidyl]-7-isopropoxy-N-[6-(trifluoromethyl)-2-pyridyl]imidazo[1,2-a]pyridine-6-carboxamide

¹H NMR (400 MHz, DMSO-d₆) δ=11.14 (br s, 1H), 10.55 (s, 1H), 9.70-9.49 (m, 1H), 9.24-9.08 (m, 1H), 8.59-8.40 (m, 1H), 8.19 (t, J=8.0 Hz, 1H), 8.02-7.86 (m, 1H), 7.78-7.68 (m, 1H), 7.49-7.37 (m, 1H), 7.29-7.24 (m, 1H), 7.18-7.12 (m, 1H), 5.05-4.91 (m, 1H), 4.41 (br s, 2H), 3.98-3.94 (m, 3H), 3.91 (br t, J=6.8 Hz, 2H), 3.80-3.72 (m, 3H), 3.65 (br dd, J=2.7, 9.0 Hz, 4H), 3.14 (br s, 4H), 2.75 (br t, J=6.8 Hz, 3H), 2.37-2.19 (m, 3H), 2.11-1.86 (m, 2H), 1.42 (br d, J=5.6 Hz, 6H). LC-MS (ES⁺): m/z 834.3 [M+H]⁺.

### Example 108 Compound of Example 108 was Prepared Substantially Following the Synthesis of Example 103

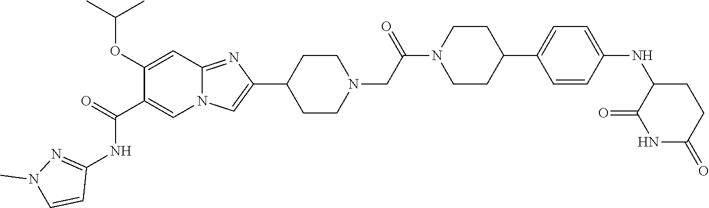 [SMILES: CC(C)Oc1cc2nc(C3CCN(CC(=O)N4CCC(c5ccc(NC6CCC(=O)NC6=O)cc5)CC4)CC3)cn2cc1C(=O)Nc1ccn(C)n1]

### 2-[1-[2-[4-[4-[(2,6-dioxo-3-piperidyl)amino]phenyl]-1-piperidyl]-2-oxo-ethyl]-4-piperidyl]-7-isopropoxy-N-(1-methylpyrazol-3-yl)imidazo[1,2-a]pyridine-6-carboxamide

¹H NMR (400 MHz, DMSO-d₆) δ=10.78 (s, 1H), 10.63 (br s, 1H), 9.75-9.54 (m, 1H), 9.23-9.08 (m, 1H), 7.98 (br s, 1H), 7.66 (d, J=2.0 Hz, 1H), 7.42-7.28 (m, 1H), 6.97 (br d, J=8.4 Hz, 2H), 6.70-6.58 (m, 3H), 5.06-4.91 (m, 1H), 4.52 (br d, J=12.4 Hz, 1H), 4.44-4.24 (m, 4H), 3.79 (s, 3H), 3.24-3.13 (m, 4H), 2.81-2.60 (m, 4H), 2.37-2.18 (m, 4H), 2.14-2.00 (m, 3H), 1.82 (br d, J=12.4 Hz, 2H), 1.72-1.55 (m, 2H), 1.43 (d, J=6.0 Hz, 6H), 1.00-0.92 (m, 1H). LC-MS (ES⁺): m/z 710.2 [M+H]⁺.

### Example 109 Compound of Example 109 was Prepared Substantially Following the Synthesis of Example 103

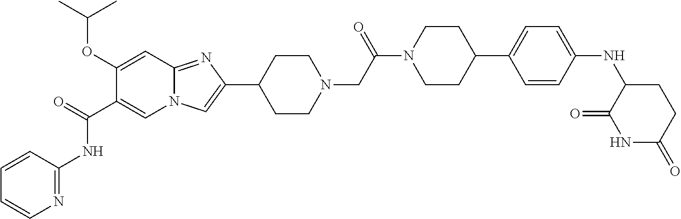 [SMILES: CC(C)Oc1cc2nc(C3CCN(CC(=O)N4CCC(c5ccc(NC6CCC(=O)NC6=O)cc5)CC4)CC3)cn2cc1C(=O)Nc1ccccn1]

### 2-[1-[2-[4-[4-[(2,6-dioxo-3-piperidyl)amino]phenyl]-1-piperidyl]-2-oxo-ethyl]-4-piperidyl]-7-isopropoxy-N-(2-pyridyl)imidazo[1,2-a]pyridine-6-carboxamide

¹H NMR (400 MHz, DMSO-d₆) δ=10.75 (s, 1H), 10.62 (s, 1H), 9.11 (s, 1H), 8.38 (br d, J=4.6 Hz, 1H), 8.25 (br d, J=8.2 Hz, 1H), 7.86 (br t, J=7.8 Hz, 1H), 7.69 (s, 1H), 7.17 (dd, J=4.9, 7.2 Hz, 1H), 7.14 (s, 1H), 6.93 (br d, J=8.3 Hz, 2H), 6.61 (br d, J=8.2 Hz, 2H), 5.66 (d, J=7.5 Hz, 1H), 4.96 (quin, J=6.0 Hz, 1H), 4.55-4.43 (m, 1H), 4.30-4.16 (m, 2H), 3.17 (d, J=5.1 Hz, 2H), 3.03 (br d, J=8.1 Hz, 2H), 2.90 (br d, J=10.6 Hz, 2H), 2.78-2.68 (m, 1H), 2.60 (br d, J=13.4 Hz, 3H), 2.54 (br d, J=4.4 Hz, 1H), 2.17-2.06 (m, 3H), 1.97 (br t, J=13.8 Hz, 2H), 1.88-1.81 (m, 1H), 1.76-1.63 (m, 4H), 1.52 (br d, J=12.2 Hz, 1H), 1.45 (s, 3H), 1.44 (s, 3H), 1.39-1.32 (m, 1H), 1.23 (s, 1H). LC-MS (ES⁺): m/z 707.4 [M+H]⁺.

### Example 110 Compound of Example 110 was Prepared Substantially Following the Synthesis of Example 103

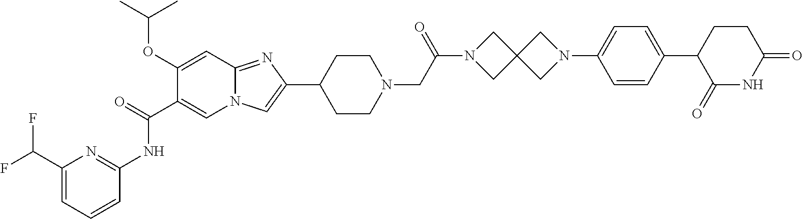 [SMILES: CC(C)Oc1cc2nc(C3CCN(CC(=O)N4CC5(C4)CN(c4ccc(C6CCC(=O)NC6=O)cc4)C5)CC3)cn2cc1C(=O)Nc1cccc(C(F)F)n1]

For step-3, DIPEA and DMF were used instead of triethylamine and DCM.

### N-[6-(difluoromethyl)-2-pyridyl]-2-[1-[2-[6-[4-(2,6-dioxo-3-piperidyl)phenyl]-2,6-diazaspiro[3.3]heptan-2-yl]-2-oxo-ethyl]-4-piperidyl]-7-isopropoxy-imidazo[1,2-a]pyridine-6-carboxamide

¹H NMR (400 MHz, DMSO-d₆) δ 10.86 (s, 1H), 10.75 (s, 1H), 9.11 (s, 1H), 8.37 (d, J=8.3 Hz, 1H), 8.07 (t, J=8.0 Hz, 1H), 7.72 (s, 1H), 7.47 (d, J=7.5 Hz, 1H), 7.13 (s, 1H), 6.89 (q, J=36.7 Hz, 2H), 6.41 (d, J=8.4 Hz, 1H), 4.95 (t, J=6.0 Hz, 1H), 4.39 (s, 2H), 4.10 (s, 2H), 3.94 (s, 3H), 3.70 (q, J=5.3 Hz, 1H), 3.0 (m, 3H), 2.62 (m, 3H), 2.03 (m, 4H), 1.67 (m, 3H), 1.46 (m, 9H), 0.93 (m, 2H). LC-MS (ES⁺): m/z 755.12 [M+H]⁺.

### Example 111 Synthesis of N-(6-(difluoromethyl)pyridin-2-yl)-2-(1-(2-(1-(4-((2,6-dioxopiperidin-3-yl)amino)phenyl)piperidin-4-ylidene)-2-fluoroethyl)piperidin-4-yl)-7-isopropoxyimidazo[1,2-a]pyridine-6-carboxamide

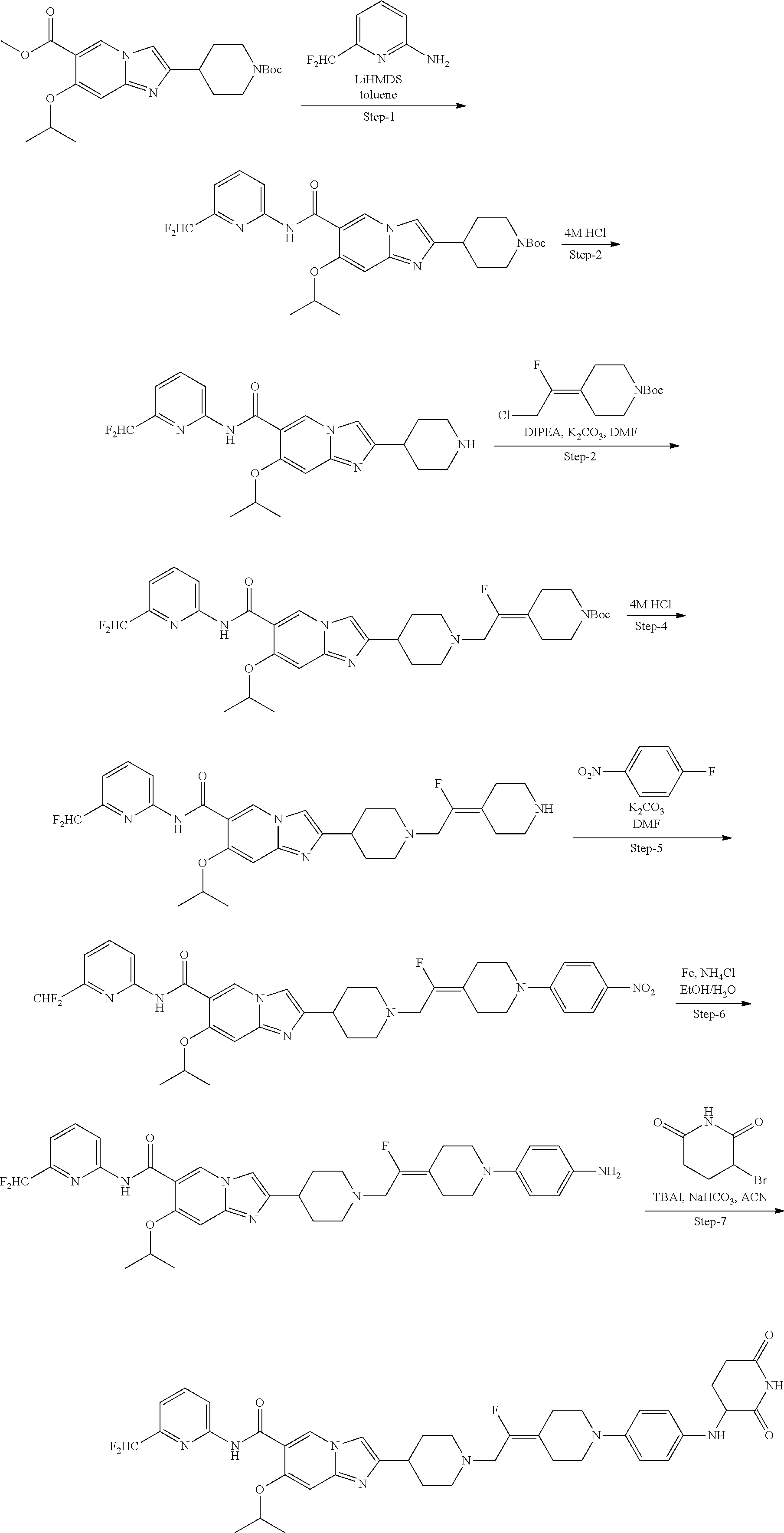

### Step-1:

To a solution of methyl 2-(1-(tert-butoxycarbonyl)piperidin-4-yl)-7-isopropoxy imidazo[1,2-a]pyridine-6-carboxylate (5 g, 11.98 mmol) and 6-(difluoromethyl)pyridin-2-amine (5.18 g, 35.93 mmol) in toluene (50 mL) was added lithium bis(trimethylsilyl)azanide (1 M, 35.93 mL) at 0° C. under nitrogen atmosphere. The mixture was stirred at 0° C. for 1 hour, then warmed up to 25° C. and stirred for an additional 3 hours. After complete consumption of the reactant as shown by LC-MS, the reaction mixture was diluted with water (100 mL) and extracted with ethyl acetate (100 mL×3). The combined organic layers were washed with brine (100 mL), dried over anhydrous sodium sulfate, filtered, and concentrated under reduced pressure. The residue was purified by column chromatography (silica gel, petroleum ether/ethyl acetate=5/1 to 0/1). Compound tert-butyl 4-(6-((6-(difluoromethyl)pyridin-2-yl)carbamoyl)-7-isopropoxyimidazo[1,2-a]pyridin-2-yl)piperidine-1-carboxylate (4.79 g, 8.32 mmol, 69.48% yield) was obtained as a yellow solid. LC-MS (ES⁺): m/z 530.2 [M+H]⁺.

### Step-2:

To a stirred solution of tert-butyl 4-[6-[[6-(difluoromethyl)-2-pyridyl]carbamoyl]-7-isopropoxy-imidazo[1,2-a]pyridin-2-yl]piperidine-1-carboxylate (0.322 g, 608.03 μmol) in dioxane (2 mL) was added with HCl (4 M, 1.52 mL) by LCMS. After completion of the reaction as confirmed by LC-MS, the solvent was removed by vacuum and the crude product was triturated with ether to afford N-[6-(difluoromethyl)-2-pyridyl]-7-isopropoxy-2-(4-piperidyl)imidazo[1,2-a]pyridine-6-carboxamide HCl salt (280 mg, 600.96 μmol, 98.84% yield) as a solid. LC-MS (ES⁺): m/z 430.2 [M+H]⁺.

### Step-3:

To a solution of N-(6-(difluoromethyl)pyridin-2-yl)-7-isopropoxy-2-(piperidin-4-yl)imidazo[1,2-a]pyridine-6-carboxamide (1 g, 2.33 mmol) and tert-butyl 4-(2-chloro-1-fluoroethylidene)piperidine-1-carboxylate (921.16 mg, 3.49 mmol) in DMF (10 mL) was added DIPEA (902.82 mg, 6.99 mmol, 1.22 mL). The mixture was stirred at 25° C. for 20 hours. Potassium carbonate (482.72 mg, 3.49 mmol) was then added to the reaction and the mixture was stirred at 25° C. for an additional 20 hours. After LC-MS indicated complete consumption of the reactant, the reaction mixture was diluted with water (60 mL) and extracted with ethyl acetate (70 mL×3). The combined organic layers were washed with brine (60 mL), dried over anhydrous sodium sulfate, filtered, and concentrated under reduced pressure. The residue was purified by column chromatography (silica gel, DCM/MeOH=100/1 to 10/1). The product tert-butyl 4-(2-(4-(6-((6-(difluoromethyl)pyridin-2-yl)carbamoyl)-7-isopropoxyimidazo[1,2-a]pyridin-2-yl)piperidin-1-yl)-1-fluoroethylidene)piperidine-1-carboxylate (1.1 g, 1.67 mmol, 71.93% yield) was obtained as a brown solid. LC-MS (ES⁺): m/z 657.3 [M+H]⁺.

### Step-4:

To a solution of tert-butyl 4-[2-[4-[6-[[6-(difluoromethyl)-2-pyridyl]carbamoyl]-7-isopropoxy-imidazo[1,2-a]pyridin-2-yl]-1-piperidyl]-1-fluoro-ethylidene]piperidine-1-carboxylate (1.1 g, 1.67 mmol) in DCM (10 mL) was added HCl (4 M, 10 mL) and the reaction mixture was stirred at 25° C. for 1 hour. After complete consumption of the reactant as confirmed by LC-MS, the reaction mixture was concentrated under reduced pressure to remove the solvent, and the crude product was used in the next step without further purification. Compound N-(6-(difluoromethyl)pyridin-2-yl)-2-(1-(2-fluoro-2-(piperidin-4-ylidene)ethyl)piperidin-4-yl)-7-isopropoxyimidazo[1,2-a]pyridine-6-carboxamide HCl salt (1 g, 1.46 mmol, 87.02% yield) was obtained as a brown solid. LC-MS (ES⁺): m/z 557.3 [M+H]⁺.

### Step-5:

To a solution of N-[6-(difluoromethyl)-2-pyridyl]-2-[1-[2-fluoro-2-(4-piperidylidene) ethyl]-4-piperidyl]-7-isopropoxy-imidazo[1,2-a]pyridine-6-carboxamide (140 mg, 251.52 μmol) and 1-fluoro-4-nitrobenzene (106.47 mg, 754.55 μmol, 80.05 μL) in DMF (2 mL) was added potassium carbonate (104.28 mg, 754.55 μmol) and the reaction mixture was stirred at 85° C. for 16 hours. After complete consumption of the reactant as confirmed by LC-MS, the reaction mixture was diluted with water (30 mL) and extracted with ethyl acetate (20 mL×3). The combined organic layers were washed with brine (20 mL), dried over anhydrous sodium sulfate, filtered, and concentrated under reduced pressure. The residue was purified by column chromatography (silica gel, DCM/MeOH=100/1 to 10/1) to afford N-(6-(difluoromethyl)pyridin-2-yl)-2-(1-(2-fluoro-2-(1-(4-nitrophenyl)piperidin-4-ylidene)ethyl)piperidin-4-yl)-7-isopropoxyimidazo[1,2-a]pyridine-6-carboxamide (110 mg, 162.31 μmol, 64.53% yield) as a yellow oil. LC-MS (ES⁺): m/z 678.6 [M+H]⁺.

### Step-6:

To a solution of N-[6-(difluoromethyl)-2-pyridyl]-2-[1-[2-fluoro-2-[1-(4-nitrophenyl)-4-piperidylidene]ethyl]-4-piperidyl]-7-isopropoxy-imidazo[1,2-a]pyridine-6-carboxamide (100 mg, 147.55 μmol) in H₂O (0.5 mL) and EtOH (2 mL) was added Fe (45.32 mg, 811.55 μmol) and ammonium chloride (23.68 mg, 442.66 μmol). The mixture was stirred at 80° C. for 16 hours, during which the mixture turned from yellow to brown color. After consumption of the reactant as shown by LC-MS, the reaction mixture was diluted with water (30 mL) and extracted with ethyl acetate (20 mL×3). The combined organic layers were washed with brine (20 mL), dried over anhydrous sodium sulfate, filtered, and concentrated under reduced pressure. The residue was purified by column chromatography (silica gel, DCM/MeOH=100/1 to 10/1) to afford compound 2-(1-(2-(1-(4-aminophenyl)piperidin-4-ylidene)-2-fluoroethyl)piperidin-4-yl)-N-(6-(difluoromethyl)pyridin-2-yl)-7-isopropoxyimidazo[1,2-a]pyridine-6-carboxamide (65 mg, 70.25 μmol, 47.61% yield) as a brown oil. LC-MS (ES⁺): m/z 648.4 [M+H]⁺.

### Step-7:

To a solution of 2-[1-[2-[1-(4-aminophenyl)-4-piperidylidene]-2-fluoro-ethyl]-4-piperidyl]-N-[6-(difluoromethyl)-2-pyridyl]-7-isopropoxy-imidazo[1,2-a]pyridine-6-carboxamide (65 mg, 100.35 μmol) and 3-bromopiperidine-2,6-dione (28.90 mg, 150.53 μmol) in CH₃CN (2 mL) was added sodium bicarbonate (25.29 mg, 301.05 μmol) and the mixture was stirred at 80° C. for 4 hours. Then 3-bromopiperidine-2,6-dione (28.90 mg, 150.53 μmol) was added and the mixture was stirred at 80° C. for another 20 hours. After LC-MS showed complete consumption of the reactant, the reaction mixture was purified by reverse phase prep-HPLC (H₂O/CH₃CN with TFA). Compound N-(6-(difluoromethyl) pyridin-2-yl)-2-(1-(2-(1-(4-((2,6-dioxopiperidin-3-yl)amino)phenyl)piperidin-4-ylidene)-2-fluoroethyl)piperidin-4-yl)-7-isopropoxyimidazo[1,2-a]pyridine-6-carboxamide TFA salt (61.79 mg, 70.79 μmol, 70.54% yield) was obtained as a gray solid. ¹H NMR (400 MHz, DMSO-d₆) δ ppm 11.06 (s, 1H) 10.82 (s, 1H) 10.11-10.44 (m, 1H) 9.17 (s, 1H) 8.30-8.42 (m, 1H) 8.10 (t, J=8.0 Hz, 1H) 7.98 (br s, 1H) 7.51 (d, J=7.6 Hz, 1H) 7.39 (s, 1H) 7.16 (s, 2H) 6.77-7.06 (m, 1H) 6.73 (d, J=8.4 Hz, 2H) 4.92-5.04 (m, 1H) 4.30-4.37 (m, 1H) 4.16-4.26 (m, 2H) 3.67 (d, J=9.2 Hz, 4H) 3.40 (br d, J=12.47 Hz, 4H) 3.07-3.28 (m, 4H) 2.55-2.81 (m, 6H) 2.27-2.39 (m, 2H) 2.06-2.14 (m, 1H) 1.83-2.02 (m, 3H) 1.42 (br d, J=4.0 Hz, 6H). LC-MS (ES⁺): m/z 759.4 [M+H]⁺.

### Example 112 Synthesis of N-[6-(difluoromethyl)-2-pyridyl]-2-[1-[2-[1-[5-(2,6-dioxo-3-piperidyl)-3-fluoro-2-pyridyl]-4-piperidylidene]-2-fluoro-ethyl]-4-piperidyl]-7-isopropoxy-imidazo[1,2-a]pyridine-6-carboxamide

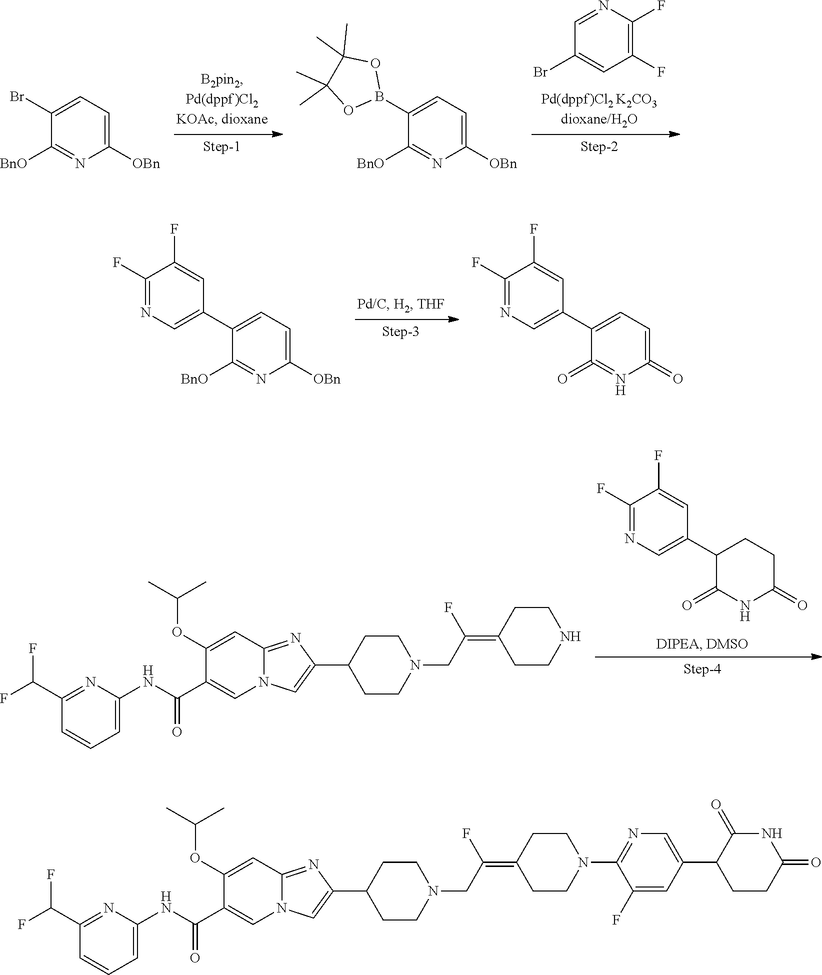

### Step-1:

In a 250 mL round bottom flask, a solution of 2,6-dibenzyloxy-3-bromo-pyridine (15 g, 40.51 mmol) in 1,4 dioxane (151.86 mL) were added 4,4,5,5-tetramethyl-2-(4,4,5,5-tetramethyl-1,3,2-dioxaborolan-2-yl)-1,3,2-dioxaborolane (15.43 g, 60.77 mmol) and potassium acetate (9.94 g, 101.29 mmol) at room temperature under argon atmosphere. The reaction mixture was degassed with argon for 20 minute before, cyclopentyl(diphenyl)phosphane dichloromethane dichloropalladium iron (3.31 g, 4.05 mmol) was added and the reaction was heated at 100° C. for 24 hours while monitoring with TLC and LC-MS. After completion of the reaction, the reaction mixture was filtered through celite bed and washed with ethyl acetate (150 mL). The filtrate was concentrated under reduced pressure to get the crude product, which was purified by column chromatography (silica gel 230-400 mesh, 0-10% EtOAc in pet-ether) to afford 2,6-dibenzyloxy-3-(4,4,5,5-tetramethyl-1,3,2-dioxaborolan-2-yl)pyridine (9 g, 16.61 mmol, 40.99% yield) as a thick pale yellow liquid. LC-MS (ES⁺): m/z 417.49 [M+H]⁺.

### Step-2:

A mixture of 2,6-bis(benzyloxy)-3-(4,4,5,5-tetramethyl-1,3,2-dioxaborolan-2-yl)pyridine (5 g, 11.98 mmol), 5-bromo-2,3-difluoropyridine (2.56 g, 13.18 mmol), cyclopentyl(diphenyl)phosphane; dichloropalladium;iron (876.70 mg, 1.20 mmol), potassium carbonate, anhydrous powder 325 mesh (4.97 g, 35.95 mmol) in dioxane (50 mL) and water (10 mL) was degassed with N₂ three times. The mixture was then stirred at 80° C. for 16 hours under N₂ atmosphere. After complete consumption of reactant as determined by LC-MS, the mixture was diluted with water (80 mL) and extracted with ethyl acetate (80 mL×3). The combined organic layers were washed with brine (80 mL×2), dried over anhydrous sodium sulfate, filtered, and concentrated in vacuo to give a residue, which was purified by column chromatography (silica gel, pet ether/ethyl acetate=10/1 to 10/1). Compound 2,6-bis(benzyloxy)-5′,6′-difluoro-3,3′-bipyridine (2.7 g, 6.68 mmol, 55.72% yield) was obtained as a yellow oil. LC-MS (ES⁺): m/z 405.1 [M+H]⁺.

### Step-3:

To a solution of 2,6-dibenzyloxy-3-(5,6-difluoro-3-pyridyl)pyridine (650 mg, 1.61 mmol) in THE (2 mL) was added 10 wt. % palladium on carbon (195.21 mg, 160.73 μmol) under N₂ atmosphere. The suspension was degassed with H₂ three times and then stirred under H₂ (15 Psi) at 25° C. for 4 hours. After the reactant was completely consumed as shown by TLC, the reaction mixture was filtered, and the filtrate was concentrated in vacuo to give a solid, which was purified by column chromatography (silica gel, petroleum ether/ethyl acetate=50/1 to 1/1). Compound 3-(5,6-difluoropyridin-3-yl)piperidine-2,6-dione (400 mg, 1.44 mmol, 89.36% yield) was obtained as a white solid. LC-MS (ES⁺): m/z 227.1 [M+H]⁺.

### Step-4:

To a solution of N-(6-(difluoromethyl)pyridin-2-yl)-2-(1-(2-fluoro-2-(piperidin-4-ylidene)ethyl)piperidin-4-yl)-7-isopropoxyimidazo[1,2-a]pyridine-6-carboxamide (123.05 mg, 221.06 μmol) and diisopropylethylamine (85.71 mg, 663.19 μmol, 115.52 μL) in DMSO (1 mL) was added 3-(5,6-difluoro-3-pyridyl)piperidine-2,6-dione (50 mg, 221.06 μmol) in one portion at 25° C. The resulting mixture was stirred at 100° C. for 2 hours under microwave condition. Progress of the reaction was monitored by LC-MS. After 30% of the desired product was detected, the reaction mixture was filtered and the residue was purified by reverse phase prep-HPLC (flow: 25 mL/min; gradient: 21-51% water (0.1% formic acid/TFA) in MeCN over 7 min; column: 3_Phenomenex Luna C18 75×30 mm×3 um). Compound N-[6-(difluoromethyl)-2-pyridyl]-2-[1-[2-[1-[5-(2,6-dioxo-3-piperidyl)-3-fluoro-2-pyridyl]-4-piperidylidene]-2-fluoro-ethyl]-4-piperidyl]-7-isopropoxy-imidazo[1,2-a]pyridine-6-carboxamide TFA salt (18.94 mg, 18.90 μmol, 8.55% yield) was obtained as a brown gum. ¹H NMR (400 MHz, DMSO-d₆) δ=11.05 (br s, 1H), 11.00-10.95 (m, 1H), 10.88 (s, 1H), 9.16 (s, 1H), 8.48-8.27 (m, 1H), 8.14-8.01 (m, 2H), 7.95-7.85 (m, 1H), 7.95-7.88 (m, 1H), 7.51 (d, J=7.8 Hz, 2H), 7.35 (br s, 1H), 7.07-6.71 (m, 1H), 5.05-4.91 (m, 1H), 4.28-4.14 (m, 2H), 4.09-4.04 (m, 1H), 3.88 (dd, J=4.8, 12.8 Hz, 1H), 3.58 (br s, 2H), 3.22-3.09 (m, 4H), 2.78-2.67 (m, 2H), 2.61-2.55 (m, 2H), 2.40-2.14 (m, 6H), 2.09-1.86 (m, 4H), 1.42 (br d, J=4.0 Hz, 6H). LC-MS (ES⁺): m/z 763.3 [M+H]⁺.

### Example 113 Synthesis of N-(1-cyclopropyl-2-oxo-1,2-dihydropyridin-3-yl)-2-((1r,4r)-4-((4-(4-((2,6-dioxopiperidin-3-yl)oxy)phenyl)piperidin-1-yl)methyl)cyclohexyl)-6-isopropoxy-2H-indazole-5-carboxamide

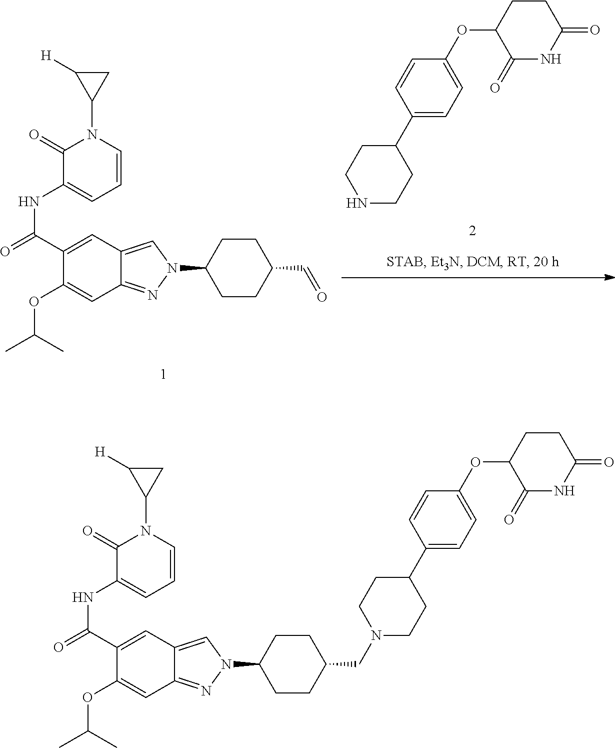 [SMILES: CC(C)Oc1cc2nn([C@H]3CC[C@H](C=O)CC3)cc2cc1C(=O)Nc1cccn(C2CC2)c1=O ; O=C1CCC(Oc2ccc(C3CCNCC3)cc2)C(=O)N1 ; CC(C)Oc1cc2nn([C@H]3CC[C@H](CN4CCC(c5ccc(OC6CCC(=O)NC6=O)cc5)CC4)CC3)cc2cc1C(=O)Nc1cccn(C2CC2)c1=O]

To a stirred solution of 3-[4-(4-piperidyl)phenoxy]piperidine-2,6-dione 2 (1.05 g, 3.24 mmol, HCl salt) in DCM (10 mL) was added TEA (2.19 g, 21.62 mmol, 3.01 mL) followed by the addition of N-(1-cyclopropyl-2-oxo-3-pyridyl)-2-(4-formylcyclohexyl)-6-isopropoxy-indazole-5-carboxamide 1 (1 g, 2.16 mmol) and stirred the reaction mixture at RT for 4 hours. Then Sodium triacetoxyborohydride (2.29 g, 10.81 mmol) was added and stirred the reaction mixture at RT for 16 hours. Upon completion, the reaction mixture was diluted with water (200 mL) and extracted with 10% methanol in DCM (3×200 mL). The combined organic layer was dried over sodium sulphate and concentrated under reduced pressure to get crude product (1.5 g). The resulting crude was purified by prep. HPLC to afford N-(1-cyclopropyl-2-oxo-3-pyridyl)-2-[4-[[4-[4-[(2,6-dioxo-3-piperidyl)oxy]phenyl]-1-piperidyl]methyl]cyclohexyl]-6-isopropoxy-indazole-5-carboxamide Example 113 (439 mg, 560.09 μmol, 25.91% yield, formic acid salt) as off white solid.

Prep. HPLC condition: Column/dimensions: SUNFIRE C18 (19*150, 5 um); Mobile phase A: 0.05% TFA in water; Mobile phase B: 100% ACN; Gradient (Time/% B): 0/15,2/15,13/39.2,13.1/98,16/98,16.1/15,18/15.; Flow rate: 17 mL/min.

LCMS (ES⁺): m/z 735.77 [M+H]⁺

¹H NMR (400 MHz, DMSO-d₆): δ 10.89 (s, 2H), 8.55 (d, J=9.6 Hz, 2H), 8.47-8.49 (m, 1H), 8.20 (s, 1H), 7.27-7.29 (m, 1H), 7.24 (s, 1H), 7.16 (d, J=8.8 Hz, 2H), 6.93 (d, J=8.8 Hz, 2H), 6.25 (t, J=7.0 Hz, 1H), 5.08-5.18 (m, 1H), 4.92-5.02 (m, 1H), 4.33-4.48 (m, 1H), 3.48-3.50 (m, 1H), 2.95 (d, J=10.8 Hz, 2H), 2.67-2.68 (m, 1H), 2.61 (s, 1H), 2.43 (s, 1H), 2.13-2.19 (m, 6H), 1.90-2.00 (m, 6H), 1.61-1.71 (m, 4H), 1.52 (d, J=6.0 Hz, 6H), 1.03-1.14 (m, 4H), 0.90-0.91 (m, 2H).

### Example 114 was Prepared Substantially Following the Synthesis of Example 113

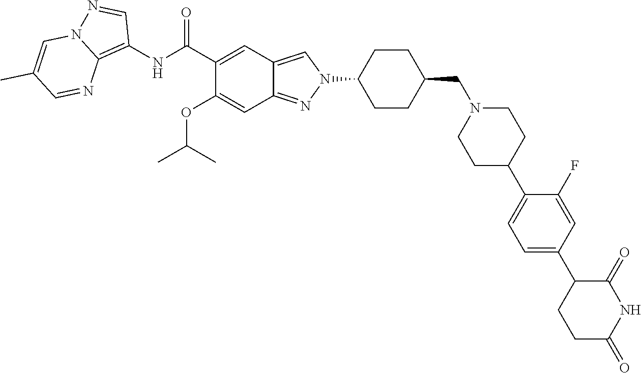 [SMILES: Cc1cnc2c(NC(=O)c3cc4cn([C@H]5CC[C@H](CN6CCC(c7ccc(C8CCC(=O)NC8=O)cc7F)CC6)CC5)nc4cc3OC(C)C)cnn2c1]

### 2-((1r,4r)-4-((4-(4-(2,6-dioxopiperidin-3-yl)-2-fluorophenyl)piperidin-1-yl)methyl)cyclohexyl)-6-isopropoxy-N-(6-methylpyrazolo[1,5-a]pyrimidin-3-yl)-2H-indazole-5-carboxamide

¹H NMR (400 MHz, DMSO-d₆): δ 10.84 (s, 1H), 10.74 (s, 1H), 8.91 (s, 1H), 8.70 (s, 1H), 8.62 (d, J=11.2 Hz, 1H), 8.56 (s, 1H), 8.45 (d, J=1.8 Hz, 1H), 7.30 (t, J=8.6 Hz, 2H), 7.03 (t, J=9.4 Hz, 2H), 5.03 (m, 1H), 4.47 (m, 1H), 3.86 (q, 1H), 3.45 (m, 1H) 2.99 (d, J=9.9 Hz, 2H), 2.79 (m, 2H), 2.66 (m, 1H), 2.34 (s, 4H), 2.22 (m, 4H), 1.98-1.68 (m, 12H), 1.54 (d, J=6.0 Hz, 6H), 1.12 (q, H). LCMS (ES⁺): m/z 735.32 [M+H]⁺.

### Example 115 was Prepared Substantially Following the Synthesis of Example 113

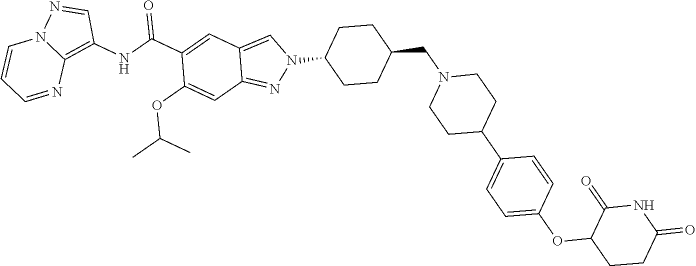 [SMILES: CC(C)Oc1cc2nn([C@H]3CC[C@H](CN4CCC(c5ccc(OC6CCC(=O)NC6=O)cc5)CC4)CC3)cc2cc1C(=O)Nc1cnn2cccnc12]

### 2-((1r,4r)-4-((4-(4-((2,6-dioxopiperidin-3-yl)oxy)phenyl)piperidin-1-yl)methyl)cyclohexyl)-6-isopropoxy-N-(pyrazolo[1,5-a]pyrimidin-3-yl)-2H- indazole-5-carboxamide

¹H NMR (400 MHz, DMSO-d₆): δ 10.93 (s, 1H), 10.75 (s, 1H), 9.08 (d, J=7.0 Hz, 1H), 8.80 (s, 1H), 8.65 (s, 1H), 8.61 (d, J=23.9 Hz, 1H), 8.53 (d, J=2.6 Hz, 1H), 7.27 (s, 1H), 7.17 (d, J=7.4 Hz, 2H), 7.05 (m, 3H), 5.17 (q, 1H), 5.04 (t, J=5.3 Hz, 1H), 4.54 (t, J=26.7 Hz, 1H), 3.70 (m, 2H), 3.17 (d, J=3.3 Hz, 1H), 3.06 (s, 3H), 2.69 (m, 4H), 2.20 (m, 4H), 1.99 (m, 7H), 1.78 (d, J=12.8 Hz, 1H), 1.55 (t, J=3.0 Hz, 6H), 1.29 (q, 2H). LCMS (ES⁺): m/z 719.40 [M+H]⁺.

### Example 116 was Prepared Substantially Following the Synthesis of Example 113

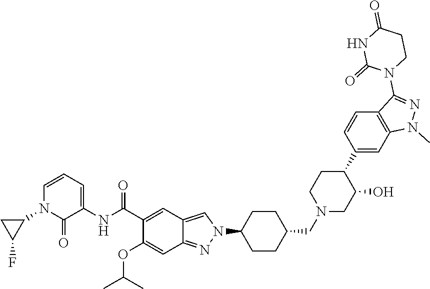

### 2-((1S,4S)-4-(((3S,4R)-4-(3-(2,4-dioxotetrahydropyrimidin-1(2H)-yl)-1-methyl-1H-indazol-6-yl)-3-hydroxypiperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 10.88 (s, 1H), 10.56 (s, 1H), 8.91-8.85(bs, 1H), 8.57 (d, J=14.8 Hz, 2H), 8.52 (dd, J=7.2, 1.6 Hz, 1H), 7.61 (d, J=8.4 Hz, 1H), 7.43-7.40 (m, 2H), 7.23 (s, 1H), 7.13 (d, J=8.8 Hz, 1H), 6.34 (t, J=7.2 Hz, 1H), 5.68-5.50 (bs, 1H), 5.01-4.97 (m, 2H), 4.99-4.97(bs, 1H), 4.38-4.28 (bs, 1H), 3.98 (s, 3H), 3.93-3.92 (m, 2H), 3.53-3.50 (m, 3H), 3.47-3.15 (m, 4H), 3.14-2.98 (m, 1H), 2.78 (t, J=6.8 Hz, 2H), 2.69-2.57 (m, 1H), 2.51 (s, 1H), 2.33-2.29 (bs, 2H), 2.00-1.90 (m, 6H), 1.52-1.45 (m, 8H). LCMS (ES⁺): m/z 808.28 [M+H]⁺.

### Example 117 was Prepared Substantially Following the Synthesis of Example 113

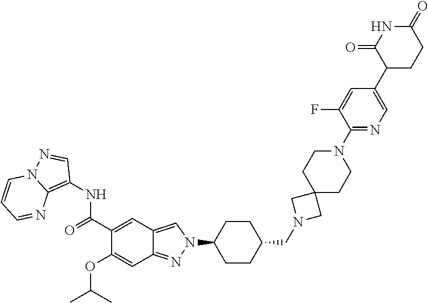 [SMILES: CC(C)Oc1cc2nn([C@H]3CC[C@H](CN4CC5(CCN(c6ncc(C7CCC(=O)NC7=O)cc6F)CC5)C4)CC3)cc2cc1C(=O)Nc1cnn2cccnc12]

### 2-((1r,4r)-4-((7-(5-(2,6-dioxopiperidin-3-yl)-3-fluoropyridin-2-yl)-2,7-diazaspiro[3.5]nonan-2-yl)methyl)cyclohexyl)-6-isopropoxy-N-(pyrazolo[1,5-a]pyrimidin-3-yl)-2H-indazole-5-carboxamide

¹H NMR (400 MHz, DMSO-d₆): δ 10.87 (s, 1H), 10.75 (s, 1H), 9.55 (d, J=7.2 Hz, 1H), 9.08 (d, J=6.8 Hz, 1H), 8.80 (s, 1H), 8.64 (d, J=6.0 Hz, 1H), 8.58 (s, 1H), 8.53 (dd, J=3.8, 1.4 Hz, 1H), 7.88 (s, 1H), 7.46 (d, J=14.4 Hz, 1H), 7.27 (s, 1H), 7.05 (dd, J=7.2, 4.0 Hz, 1H), 5.03-5.01 (m, 1H), 4.62-4.41 (m, 1H), 4.10-4.09 (m, 2H), 3.95-3.83 (m, 3H), 3.59-3.18 (m, 5H), 2.69-2.67 (m, 2H), 2.33-2.19 (m, 3H), 1.99-1.90 (m, 8H), 1.56-1.28 (m, 10H). LCMS (ES⁺): m/z 763.36[M+H]⁺

### Example 118 was Prepared Substantially Following the Synthesis of Example 113

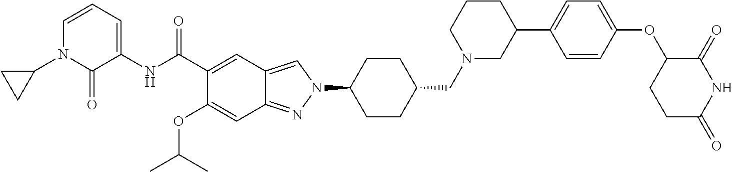 [SMILES: CC(C)Oc1cc2nn([C@H]3CC[C@H](CN4CCCC(c5ccc(OC6CCC(=O)NC6=O)cc5)C4)CC3)cc2cc1C(=O)Nc1cccn(C2CC2)c1=O]

N-(1-cyclopropyl-2-oxo-1,2-dihydropyridin-3-yl)-2-((1r,4r)-4-((3-(4-((2,6-dioxopiperidin-3-yl)oxy)phenyl)piperidin-1-yl)methyl)cyclohexyl)-6-isopropoxy-2H-indazole-5-carboxamide

LCMS (ES⁺): m/z 735.36 [M+H]⁺¹H-NMR (400 MHz, DMSO D6) δ 10.89 (s, 2H), 8.55 (s, 1H), 8.52 (s, 1H), 8.48 (dd, J=7.6, 1.6 Hz, 1H) 8.49-8.47 (m, 1H), 7.28 (dd, J=6.8, 1.6 Hz, 1H), 7.23 (s, 1H), 7.18 (d, J=8.4 Hz, 2H), 6.94 (d, J=8.4 Hz, 2H), 6.25 (t, J=7.2 Hz, 1H), 5.13 (t, J=5.2 Hz, 1H), 4.97 (t, J=6.0 Hz, 1H), 4.50-4.35 (m, 1H), 3.51-3.48 (m, 1H), 2.86-2.71 (m, 2H), 2.70-2.62 (m, 3H), 2.18-1.92 (m, 6H), 1.91-1.88 (m, 6H), 1.85-1.60 (m, 4H), 1.51 (d, J=6.0 Hz, 6H), 1.44-1.30 (m, 1H), 1.12-1.02 (m, 4H), 0.92-0.90 (m, 2H).

### Example 119 was Prepared Substantially Following the Synthesis of Example 113

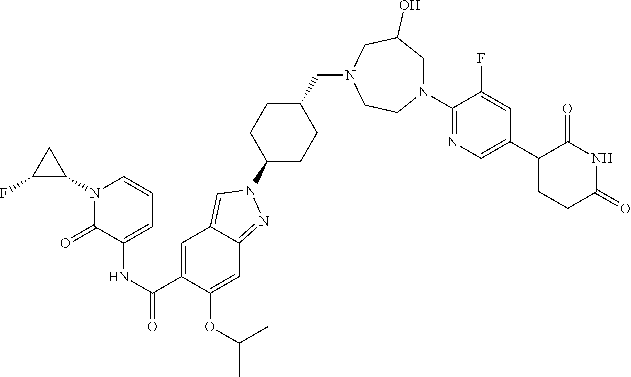 [SMILES: CC(C)Oc1cc2nn([C@H]3CC[C@H](CN4CCN(c5ncc(C6CCC(=O)NC6=O)cc5F)CC(O)C4)CC3)cc2cc1C(=O)Nc1cccn([C@H]2C[C@H]2F)c1=O]

### 2-((1r,4S)-4-((4-(5-(2,6-dioxopiperidin-3-yl)-3-fluoropyridin-2-yl)-6-hydroxy-1,4-diazepan-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 10.86 (s, 2H), 8.71 (bs, 1H), 8.58 (s, 1H), 8.54-8.50 (m, 2H), 7.85 (bs, 1H), 7.48 (d, J=11.2 Hz, 1H), 7.40 (d, J=6.8 Hz, 1H), 7.22 (s, 1H), 6.32 (d, J=7.2 Hz, 1H), 6.14 (bs, 1H), 5.17-4.93 (m, 2H), 4.53-4.37 (m, 2H), 4.10-3.35 (m, 10H), 3.25-2.96 (m, 2H), 2.73-2.53 (m, 2H), 2.36-1.87 (m, 9H), 1.69-1.54 (m, 2H), 1.53 (d, J=6.8 Hz, 6H), 1.39-1.28 (m, 2H). LCMS (ES⁺): m/z 787.22 [M+H]⁺.

### Example 120 was Prepared Substantially Following the Synthesis of Example 113

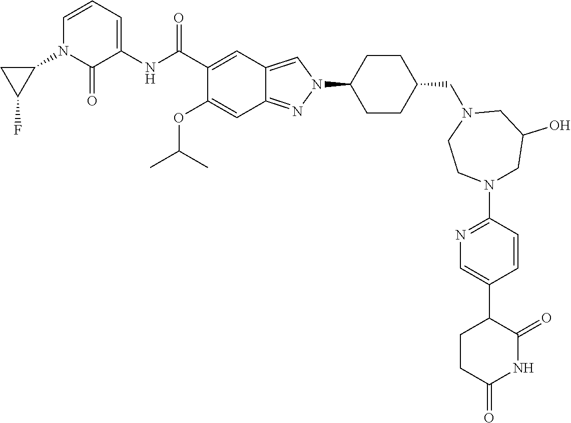 [SMILES: CC(C)Oc1cc2nn([C@H]3CC[C@H](CN4CCN(c5ccc(C6CCC(=O)NC6=O)cn5)CC(O)C4)CC3)cc2cc1C(=O)Nc1cccn([C@H]2C[C@H]2F)c1=O]

### 2-((1r,4S)-4-((4-(5-(2,6-dioxopiperidin-3-yl)pyridin-2-yl)-6-hydroxy-1,4-diazepan-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆) δ 10.87 (s, 1H), 10.84 (s, 1H), 9.08-8.76 (bs, 1H), 8.64-8.50 (m, 3H), 8.03-7.93 (m, 1H), 7.58-7.48 (m, 1H), 7.40 (d, J=6.4 Hz, 1H), 7.22 (d, J=4.8 Hz, 1H), 7.04-6.82 (m, 1H), 6.32 (d, J=6.8 Hz, 1H), 5.68 (bs, 1H), 5.18-4.94 (m, 2H), 4.55-4.36 (bs, 3H), 4.12-3.72 (bs, 5H), 3.49-2.93 (m, 7H), 2.68 (bs, 1H), 2.56 (bs, 1H), 2.21 (bs, 3H), 2.04-1.82 (bs, 5H), 1.69-1.57 (m, 2H), 1.55 (d, J=7.2 Hz, 6H), 1.35-1.27 (m, 2H). LCMS (ES⁺): m/z 769.25 [M+H]⁺

### Example 121 was Prepared Substantially Following the Synthesis of Example 113

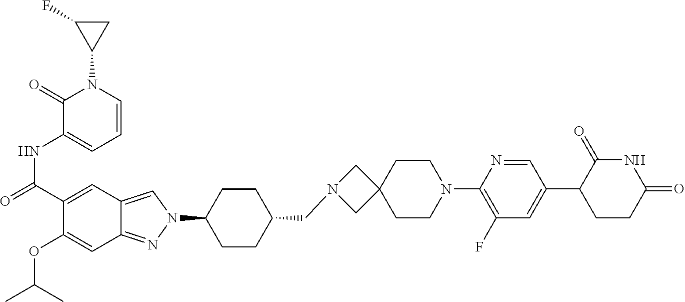 [SMILES: CC(C)Oc1cc2nn([C@H]3CC[C@H](CN4CC5(CCN(c6ncc(C7CCC(=O)NC7=O)cc6F)CC5)C4)CC3)cc2cc1C(=O)Nc1cccn([C@H]2C[C@H]2F)c1=O]

### 2-((1r,4S)-4-((7-(5-(2,6-dioxopiperidin-3-yl)-3-fluoropyridin-2-yl)-2,7-diazaspiro[3.5]nonan-2-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 10.87 (s, 2H), 9.61 (bs, 1H), 8.51-8.60 (m, 3H), 7.88 (s, 1H), 7.46 (d, J=14.4 Hz, 1H), 7.41 (d, J=6.8 Hz, 1H), 7.23 (s, 1H), 6.30 (t, J=7.2 Hz, 1H), 4.99-5.17 (m, 2H), 4.46-4.58 (m, 1H), 3.83-4.12 (m, 5H), 3.18-3.46 (m, 7H), 2.55-2.69 (m, 2H), 2.18-2.26 (m, 3H), 1.89-1.95 (m, 8H), 1.27-1.58 (m, 12H). LCMS (ES⁺): m/z 797.56 [M+H]⁺.

### Example 122 was Prepared Substantially Following the Synthesis of Example 113

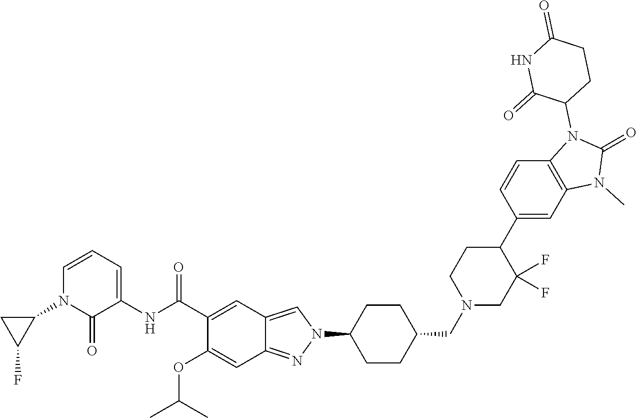 [SMILES: CC(C)Oc1cc2nn([C@H]3CC[C@H](CN4CCC(c5ccc6c(c5)n(C)c(=O)n6C5CCC(=O)NC5=O)C(F)(F)C4)CC3)cc2cc1C(=O)Nc1cccn([C@H]2C[C@H]2F)c1=O]

### 2-((1r,4S)-4-((4-(1-(2,6-dioxopiperidin-3-yl)-3-methyl-2-oxo-2,3-dihydro-1H-benzo[d]imidazol-5-yl)-3,3-difluoropiperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

LCMS (ES⁺): m/z 843.20 [M+H]⁺. ¹H-NMR (400 MHz, DMSO-D6) δ 11.10 (s, 1H), 10.88 (s, 1H), 8.57 (d, J=12.4 Hz, 2H), 8.52 (dd, J=7.4, 1.4 Hz, 1H), 7.40 (d, J=6.0 Hz, 1H), 7.24 (s, 1H), 7.15 (br s, 1H), 7.08 (br s, 1H), 6.99 (d, J=8.0 Hz, 1H), 6.30 (t, J=7.2 Hz, 1H), 5.30-5.45 (m, 1H), 4.97-5.01 (m, 2H), 4.43 (br s, 1H), 2.91-3.17 (m, 7H), 2.61-2.72 (m, 3H), 1.91-1.99 (m, 6H), 2.09-1.95 (m, 6H), 1.52-1.65 (m, 2H), 1.44 (d, J=8.4 Hz, 6H), 1.36-1.340 (m, 1H), 1.16-1.19 (m, 3H).

### Example 123 was Prepared Substantially Following the Synthesis of Example 113

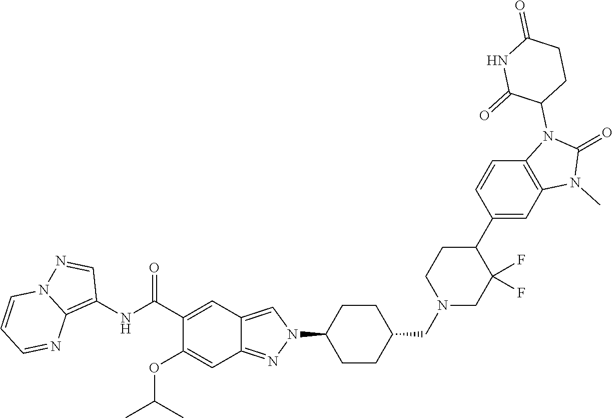 [SMILES: CC(C)Oc1cc2nn([C@H]3CC[C@H](CN4CCC(c5ccc6c(c5)n(C)c(=O)n6C5CCC(=O)NC5=O)C(F)(F)C4)CC3)cc2cc1C(=O)Nc1cnn2cccnc12]

### 2-((1r,4r)-4-((4-(1-(2,6-dioxopiperidin-3-yl)-3-methyl-2-oxo-2,3-dihydro-1H-benzo[d]imidazol-5-yl)-3,3-difluoropiperidin-1-yl)methyl)cyclohexyl)-6-isopropoxy-N-(pyrazolo[1,5-a]pyrimidin-3-yl)-2H-indazole-5-carboxamide

¹H NMR (400 MHz, DMSO-d₆): δ 11.11 (s, 1H), 10.76 (s, 1H), 9.08 (dd, J=7.2, 1.6 Hz, 1H), 8.80 (s, 1H), 8.63 (s, 1H), 8.57 (s, 1H), 8.53 (dd, J=4.0, 1.6 Hz, 1H), 7.28 (s, 1H), 7.10-7.09 (m, 2H), 7.06-7.03 (m, 1H), 6.96 (t, J=10.0 Hz, 2H), 5.40-5.36 (m, 1H), 5.07-5.01 (m, 1H), 4.48 (br s, 1H), 3.51 (s, 3H), 3.36 (s, 4H), 3.18-3.16 (m, 2H), 2.95-2.65 (m, 4H), 2.42 (s, 1H), 2.21-2.19 (m, 3H), 2.01-1.99 (m, 6H), 1.55 (d, J=6.0 Hz, 6H). LCMS (ES⁺): m/z 809.19 [M+H]⁺.

### Example 124 was Prepared Substantially Following the Synthesis of Example 113

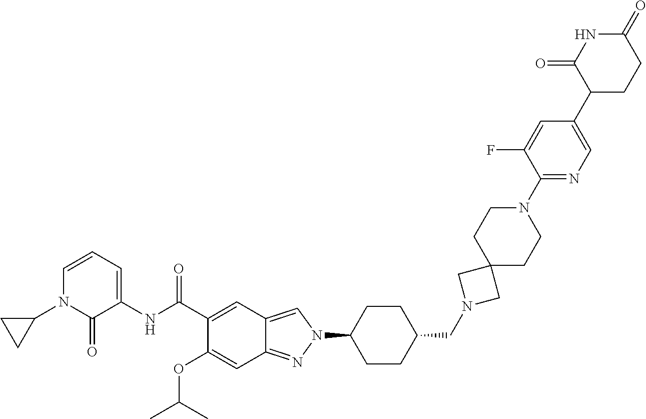 [SMILES: CC(C)Oc1cc2nn([C@H]3CC[C@H](CN4CC5(CCN(c6ncc(C7CCC(=O)NC7=O)cc6F)CC5)C4)CC3)cc2cc1C(=O)Nc1cccn(C2CC2)c1=O]

### N-(1-cyclopropyl-2-oxo-1,2-dihydropyridin-3-yl)-2-((r,4r)-4-((7-(5-(2,6-dioxopiperidin-3-yl)-3-fluoropyridin-2-yl)-2,7-diazaspiro[3.5]nonan-2-yl)methyl)cyclohexyl)-6-isopropoxy-2H-indazole-5-carboxamide

¹H-NMR (400 MHz, DMSO D6) δ 10.88 (s, 1H), 10.86 (s, 1H), 9.57 (s, 1H), 8.58 (dd, J=11.8, 5.8 Hz, 2H), 8.48 (d, J=7.2 Hz, 1H), 7.88 (s, 1H), 7.45-7.48 (m, 1H), 7.29 (dd, J=6.8, 1.6 Hz, 1H), 7.22 (s, 1H), 6.26 (t, J=7.2 Hz, 1H), 4.99-5.03 (m, 1H), 4.45-4.55 (m, 1H), 4.08-4.12 (m, 2H), 3.86-3.95 (m, 3H), 3.46-3.52 (m, 3H), 3.17-3.20 (m, 4H), 2.54-2.55 (m, 2H), 2.18-2.22 (m, 3H), 1.89-1.99 (m, 8H), 1.60 (br s, 2H), 1.52 (d, J=6.0 Hz, 6H), 1.24-1.26 (m, 2H), 1.04-1.05 (m, 2H), 0.90-0.91 (m, 2H). LCMS (ES⁺): m/z 779.51 [M+H]⁺

### Example 125 was Prepared Substantially Following the Synthesis of Example 113

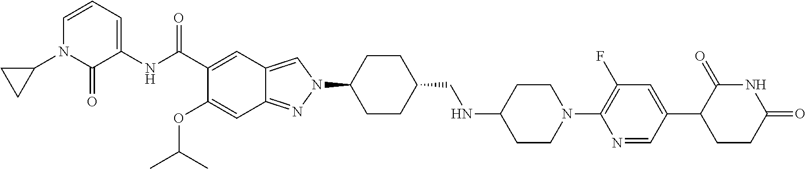 [SMILES: CC(C)Oc1cc2nn([C@H]3CC[C@H](CNC4CCN(c5ncc(C6CCC(=O)NC6=O)cc5F)CC4)CC3)cc2cc1C(=O)Nc1cccn(C2CC2)c1=O]

### N-(1-cyclopropyl-2-oxo-1,2-dihydropyridin-3-yl)-2-((1r,4r)-4-(((1-(5-(2,6-dioxopiperidin-3-yl)-3-fluoropyridin-2-yl)piperidin-4-yl)amino)methyl)cyclohexyl)-6-isopropoxy-2H-indazole-5-carboxamide

¹H NMR (400 MHz, DMSO-d₆): δ 10.85 (s, 2H), 8.55 (d, J=6.0 Hz, 2H), 8.48 (dd, J=7.4, 1.4 Hz, 1H), 8.37 (s, 1H), 7.87 (s, 1H), 7.43 (dd, J=14.4, 1.6 Hz, 1H), 7.28 (dd, J=7.2, 1.6 Hz, 1H), 7.24 (s, 1H), 6.25 (t, J=7.2 Hz, 1H), 4.99-4.97 (m, 1H), 4.45-4.38 (m, 1H), 3.90-3.82 (m, 3H), 3.51-3.48 (m, 1H), 2.95-2.89 (m, 3H), 2.69-2.63 (m, 5H), 2.17-2.14 (m, 1H), 1.99 (d, J=4.0 Hz, 2H), 1.97-1.88 (m, 7H), 1.52 (d, J=6.0 Hz, 6H), 1.48 (s, 1H), 1.17-1.14 (m, 2H), 1.05-1.03 (m, 2H), 1.02-0.98 (m, 2H), 0.92-0.90 (m, 2H). LCMS (ES⁺): m/z 753.54 [M+H]⁺.

### Example 126 was Prepared Substantially Following the Synthesis of Example 113

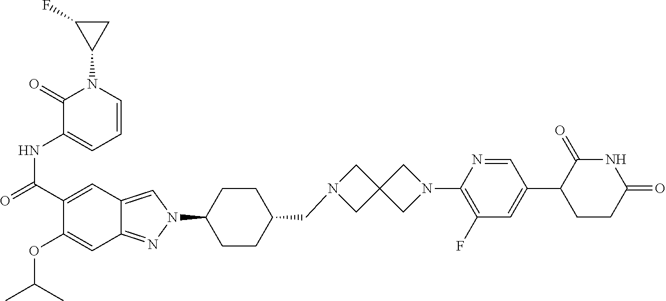 [SMILES: CC(C)Oc1cc2nn([C@H]3CC[C@H](CN4CC5(C4)CN(c4ncc(C6CCC(=O)NC6=O)cc4F)C5)CC3)cc2cc1C(=O)Nc1cccn([C@H]2C[C@H]2F)c1=O]

### 2-((1r,4S)-4-((6-(5-(2,6-dioxopiperidin-3-yl)-3-fluoropyridin-2-yl)-2,6-diazaspiro[3.3]heptan-2-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 10.87 (s, 1H), 10.83 (s, 1H), 8.58 (s, 1H), 8.56 (s, 1H), 8.51 (dd, J=7.4, 1.4 Hz, 1H), 8.14 (s, 1H), 7.78 (s, 1H), 7.39 (s, 1H), 7.36 (d, J=1.6 Hz, 1H), 7.24 (s, 1H), 6.30 (t, J=7.2 Hz, 1H), 5.16-4.92 (m, 2H), 4.46-4.35 (m, 1H), 4.13 (s, 4H), 3.80 (dd, J=12.4, 4.8 Hz, 1H), 3.42-3.48 (m, 5H), 2.51-2.68 (m, 3H), 2.41 (brs, 1H), 2.25-2.15 (m, 3H), 2.01-1.91 (m, 5H), 1.68-1.54 (m, 2H), 1.52 (d, J=6.8 Hz, 6H), 1.49-1.38 (m, 2H), 1.12-1.16 (m, 2H). LCMS (ES⁺): m/z 769.68 [M+H]⁺.

### Example 127 was Prepared Substantially Following the Synthesis of Example 113

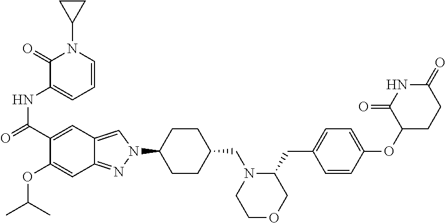 [SMILES: CC(C)Oc1cc2nn([C@H]3CC[C@H](CN4CCOC[C@H]4Cc4ccc(OC5CCC(=O)NC5=O)cc4)CC3)cc2cc1C(=O)Nc1cccn(C2CC2)c1=O]

### N-(1-cyclopropyl-2-oxo-1,2-dihydropyridin-3-yl)-2-((S,4r)-4-(((3S)-3-(4-((2,6-dioxopiperidin-3-yl)oxy)benzyl)morpholino)methyl)cyclohexyl)-6-isopropoxy-2H-indazole-5-carboxamide

¹H NMR (400 MHz, DMSO-d₆): δ 10.89 (s, 2H), 8.56 (d, J=9.2 Hz, 2H), 8.48 (dd, J=7.2, 1.6 Hz, 1H), 7.28 (dd, J=6.8, 1.6 Hz, 2H), 7.24 (s, 2H), 7.12 (d, J=8.4 Hz, 2H), 7.02 (br s, 2H), 6.94 (d, J=8.4 Hz, 1H), 6.25 (t, J=7.2 Hz, 1H), 5.20-5.10 (m, 1H), 4.99-4.98 (m, 1H), 4.50-4.45 (m, 1H), 3.60 (s, 2H), 3.51-3.47 (m, 1H), 3.40-3.32 (m, 2H), 2.71-2.51 (m, 7H), 2.44-1.75 (m, 8H), 1.52 (d, J=6.0 Hz, 6H), 1.14-1.02 (m, 4H), 0.92-0.90 (m, 2H). LCMS (ES⁺): m/z 751.24 [M+H]⁺.

### Example 128 was Prepared Substantially Following the Synthesis of Example 113

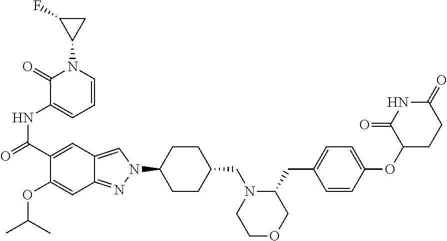 [SMILES: CC(C)Oc1cc2nn([C@H]3CC[C@H](CN4CCOC[C@H]4Cc4ccc(OC5CCC(=O)NC5=O)cc4)CC3)cc2cc1C(=O)Nc1cccn([C@H]2C[C@H]2F)c1=O]

### 2-((1R,4S)-4-(((3R)-3-(4-((2,6-dioxopiperidin-3-yl)oxy)benzyl)morpholino)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 10.88 (s, 2H), 8.57-8.48 (m, 3H), 7.40 (d, J=7.2 Hz, 1H), 7.24 (s, 1H), 7.12 (d, J=8.4 Hz, 2H), 6.94 (d, J=8.8 Hz, 2H), 6.32 (t, J=7.2 Hz, 1H), 5.14-4.97 (m, 3H), 4.42 (brs, 1H), 3.60 (brs, 2H), 3.46-3.38 (m, 2H), 3.32-3.25 (m, 1H), 2.89-2.46 (m, 7H), 2.23-2.12 (m, 6H), 2.05-1.86 (m, 4H), 1.68-1.56 (m, 2H), 1.52 (d, J=6.8 Hz, 6H), 1.15-1.05 (m, 2H). LCMS (ES⁺): m/z 769.62 [M+H]⁺.

### Example 129 was Prepared Substantially Following the Synthesis of Example 113

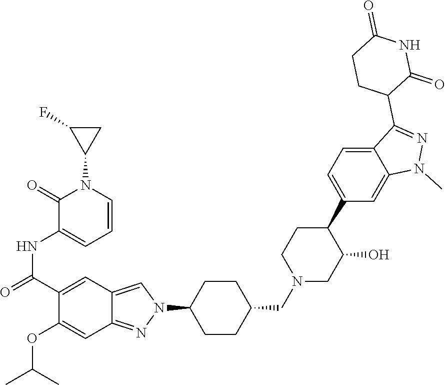 [SMILES: CC(C)Oc1cc2nn([C@H]3CC[C@H](CN4CC[C@@H](c5ccc6c(C7CCC(=O)NC7=O)nn(C)c6c5)[C@H](O)C4)CC3)cc2cc1C(=O)Nc1cccn([C@H]2C[C@H]2F)c1=O]

### 2-((1S,4S)-4-(((3S,4S)-4-(3-(2,6-dioxopiperidin-3-yl)-1-methyl-1H-indazol-6-yl)-3-hydroxypiperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 10.88 (s, 1H), 10.87 (s, 1H), 8.84 (bs, 1H), 8.60 (s, 1H), 8.56 (s, 1H), 8.51 (d, J=6.0 Hz, 1H), 7.72 (d, J=8.8 Hz, 1H), 7.65 (s, 1H), 7.40 (d, J=8.8 Hz, 1H), 7.22 (s, 2H), 6.32 (t, J=7.2 Hz, 1H), 5.69-5.38 (m, 1H), 5.16-4.94 (m, 2H), 4.52 (bs, 1H), 4.39-4.32 (m, 1H), 4.06-3.97 (m, 3H), 3.56 (bs, 4H), 3.16 (bs, 3H), 2.86-2.56 (m, 2H), 2.47-2.10 (m, 5H), 2.08-1.86 (m, 7H), 1.69-1.53 (m, 2H), 1.52 (d, J=6.8 Hz, 6H), 1.38-1.20 (m, 2H). LCMS (ES⁺): m/z 807.25 [M+H]⁺.

### Example 130 was Prepared Substantially Following the Synthesis of Example 113

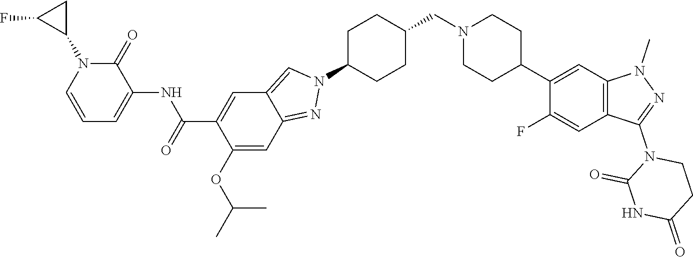 [SMILES: CC(C)Oc1cc2nn([C@H]3CC[C@H](CN4CCC(c5cc6c(cc5F)c(N5CCC(=O)NC5=O)nn6C)CC4)CC3)cc2cc1C(=O)Nc1cccn([C@H]2C[C@H]2F)c1=O]

### 2-((1r,4S)-4-((4-(3-(2,4-dioxotetrahydropyrimidin-1(2H)-yl)-5-fluoro-1-methyl-1H-indazol-6-yl)piperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 10.88 (s, 1H), 10.57 (s, 1H), 8.91 (br s, 1H), 8.59 (s, 1H), 8.56 (s, 1H), 8.52 (dd, J=7.4, 1.4 Hz, 1H), 7.40-7.49 (m, 3H), 7.23 (s, 1H), 6.31 (t, J=7.2 Hz, 1H), 5.18-5.00 (m, 2H), 4.50 (br s, 1H), 4.02 (s, 3H), 3.89 (t, J=6.6 Hz, 2H), 3.45-3.48 (m, 3H), 3.17-3.22 (m, 3H), 3.09 (br s, 2H), 2.74 (t, J=6.6 Hz, 2H), 1.98-2.33 (m, 11H), 1.70-1.52 (m, 8H), 1.29-1.32 (m, 2H). LCMS (ES⁺): m/z 810.14 [M+H]⁺.

### Example 131 was Prepared Substantially Following the Synthesis of Example 113

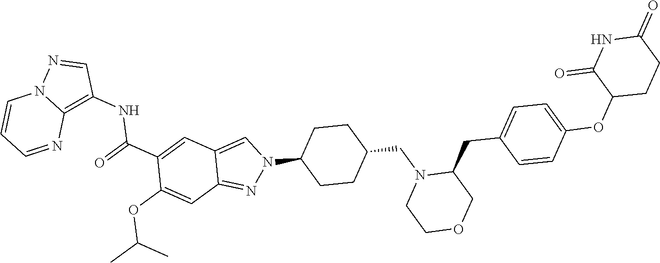 [SMILES: CC(C)Oc1cc2nn([C@H]3CC[C@H](CN4CCOC[C@@H]4Cc4ccc(OC5CCC(=O)NC5=O)cc4)CC3)cc2cc1C(=O)Nc1cnn2cccnc12]

### 2-((1R,4r)-4-(((3R)-3-(4-((2,6-dioxopiperidin-3-yl)oxy)benzyl)morpholino)methyl)cyclohexyl)-6-isopropoxy-N-(pyrazolo[1,5-a]pyrimidin-3-yl)-2H-indazole-5-carboxamide

¹H NMR (400 MHz, DMSO-d₆): δ 10.91 (s, 1H), 10.75 (s, 1H), 9.07 (dd, J=7.2, 1.6 Hz, 1H), 8.80 (s, 1H), 8.62-8.52 (m, 3H), 7.28 (S, 1H), 7.13-6.93 (m, 5H), 5.16-5.02 (m, 2H), 4.45 (m, 1H), 3.60 (s, 2H), 3.41-3.25 (m, 2H), 2.90-2.50 (m, 7H), 2.29-1.89 (m, 10H), 1.61-1.66 (m, 1H), 1.55 (d, J=6.0 Hz, 6H), 1.15 (m, 2H). LCMS (ES⁺): m/z 735.58 [M+H]⁺.

### Example 132 was Prepared Substantially Following the Synthesis of Example 113

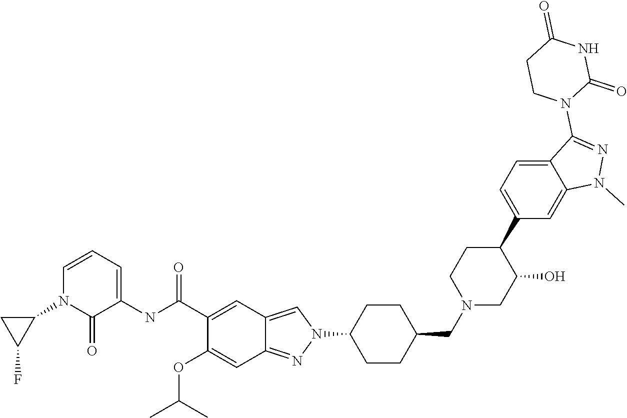

### 2-((1S,4S)-4-(((3S,4S)-4-(3-(2,4-dioxotetrahydropyrimidin-1(2H)-yl)-1-methyl-1H-indazol-6-yl)-3-hydroxypiperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 10.88 (s, 1H), 10.54 (s, 1H), 8.59 (s, 1H), 8.56 (s, 1H), 8.52 (dd, J=7.4, 1.4 Hz, 1H), 7.55 (d, J=8.4 Hz, 1H), 7.42 (s, 1H), 7.41 (d, J=6.8 Hz, 1H), 7.23 (s, 1H), 7.07 (d, J=8.8 Hz, 1H), 6.35 (t, J=7.2 Hz, 1H), 4.95-5.12 (m, 2H), 3.89-3.97 (m, 5H), 3.80-3.82 (m, 1H), 3.43-3.48 (m, 1H), 2.90-3.20 (m, 2H), 2.53-2.79 (m, 4H), 2.10-2.50 (m, 5H), 1.93-2.17 (m, 5H), 1.57-1.89 (m, 3H), 1.50-1.52 (m, 8H), 1.17-1.24 (m, 3H). LCMS (ES⁺): m/z 808.71 [M+H]⁺

### Example 133 was Prepared Substantially Following the Synthesis of Example 113

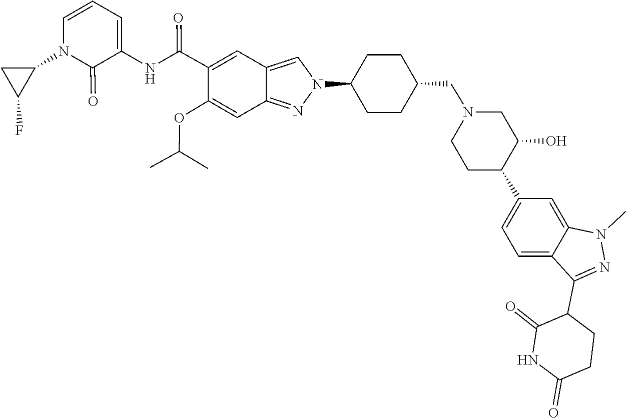 [SMILES: CC(C)Oc1cc2nn([C@H]3CC[C@H](CN4CC[C@@H](c5ccc6c(C7CCC(=O)NC7=O)nn(C)c6c5)[C@@H](O)C4)CC3)cc2cc1C(=O)Nc1cccn([C@H]2C[C@H]2F)c1=O]

### 2-((1R,4S)-4-(((3R,4S)-4-(3-(2,6-dioxopiperidin-3-yl)-1-methyl-1H-indazol-6-yl)-3-hydroxypiperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆) δ:11.88 (br s, 1H), 10.89 (s, 2H), 8.57 (d, J=7.2 Hz, 1H), 8.52 (dd, J=7.2, 1.6 Hz, 2H), 7.58 (d, J=8.4 Hz, 1H), 7.45 (s, 1H), 7.40 (dd, J=6.4, 0.8 Hz, 1H), 7.25 (s, 1H), 7.10 (d, J=8.4 Hz, 1H), 6.30 (t, J=7.2 Hz, 1H), 5.00-4.97 (m, 2H), 4.44-4.50 (m, 1H), 4.34-4.31 (m, 1H), 3.99-3.96 (m, 4H), 3.83 (br s, 1H), 3.50-3.46 (m, 1H), 2.98 (d, J=10.4 Hz, 2H), 2.67-2.61 (m, 1H), 2.53-2.50 (m, 2H), 2.36-2.32 (m, 8H), 2.23-2.19 (m, 3H), 2.06-2.04 (m, 2H), 1.52-1.50 (m, 10H), 1.15-1.10 (m, 2H). LCMS (ES⁺): m/z 807.64 [M+H]⁺

### Example 134 was Prepared Substantially Following the Synthesis of Example 113

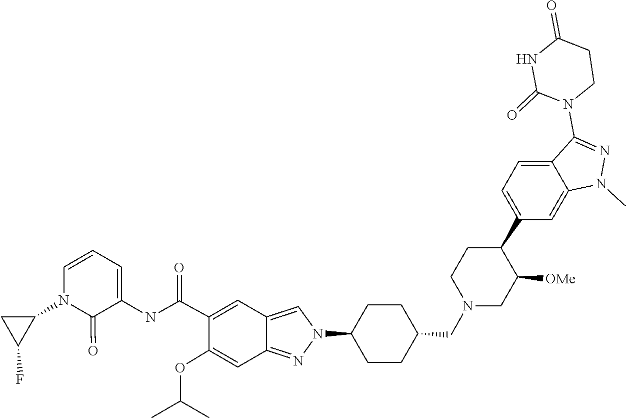

### 2-((1R,4S)-4-(((3R,4S)-4-(3-(2,4-dioxotetrahydropyrimidin-1(2H)-yl)-1-methyl-1H-indazol-6-yl)-3-methoxypiperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H-NMR (400 MHz, DMSO-d₆): δ 10.85 (s, 1H), 10.56 (s, 1H), 8.95 (br s, 1H), 8.59 (s, 1H), 8.55 (s, 1H), 8.52 (dd, J=7.6, 1.6 Hz, 1H), 7.62 (d, J=8.4 Hz, 1H), 7.44-7.40 (m, 2H), 7.22 (s, 1H), 7.15 (d, J=8.4 Hz, 1H), 6.31 (t, J=7.2 Hz, 1H), 5.18-5.00 (m, 2H), 4.58-4.46 (m, 1H), 3.99-3.88 (m, 7H), 3.48-3.43 (m, 2H), 3.24-3.12 (m, 7H), 3.04-2.95 (m, 1H), 2.75 (t, J=6.8 Hz, 2H), 2.54-2.51 (m, 1H), 2.33-2.20 (m, 2H), 2.05-1.95 (m, 6H), 1.52-1.32 (m, 10H). LCMS (ES⁻): m/z 820.25 [M−H]⁻

### Example 135 was Prepared Substantially Following the Synthesis of Example 113

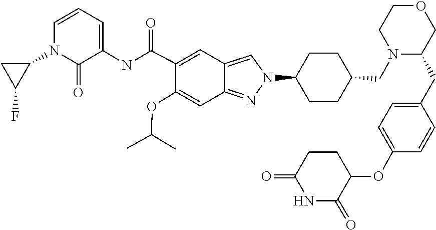

### 2-((1S,4S)-4-(((3S)-3-(4-((2,6-dioxopiperidin-3-yl)oxy)benzyl)morpholino)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆) δ 10.92 (s, 1H), 10.88 (s, 1H), 8.57-8.51 (m, 3H), 7.40 (d, J=6.0 Hz, 1H), 7.24 (s, 1H), 7.12 (d, J=8.8 Hz, 2H), 6.94 (d, J=8.8 Hz, 2H), 6.32 (t, J 7.2 Hz, 1H), 5.14-5.13 (m, 1H), 5.01-4.97 (m, 2H), 4.50-4.30 (m, 1H), 3.72-3.69 (bs, 2H), 3.54-3.38 (m, 2H), 3.32-3.25 (m, 2H), 2.88 (d, J=9.6 Hz, 1H), 2.71-2.51 (m, 5H), 2.22-2.12 (m, 6H), 2.20-2.02 (m, 1H), 1.93-1.88 (m, 3H), 1.63-1.50 (m, 9H), 1.49-1.33 (m, 2H). LCMS (ES⁺): m/z 769.68 [M+H]⁺

### Example 136 was Prepared Substantially Following the Synthesis of Example 113

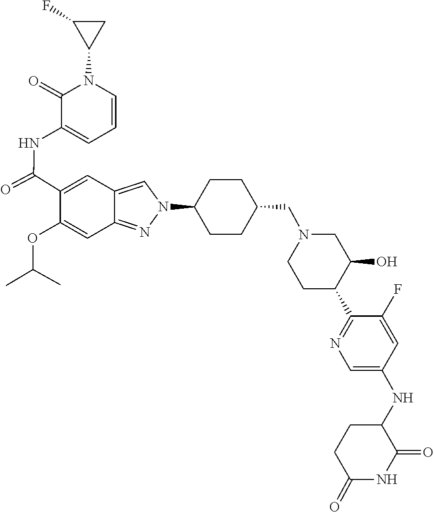 [SMILES: CC(C)Oc1cc2nn([C@H]3CC[C@H](CN4CC[C@@H](c5ncc(NC6CCC(=O)NC6=O)cc5F)[C@H](O)C4)CC3)cc2cc1C(=O)Nc1cccn([C@H]2C[C@H]2F)c1=O]

### 2-((1S,4S)-4-(((3S,4S)-4-(5-((2,6-dioxopiperidin-3-yl)amino)-3-fluoropyridin-2-yl)-3-hydroxypiperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H-NMR (400 MHz, DMSO-d₆) δ 10.95 (s, 1H), 10.85 (s, 1H), 9.14 (s, 1H), 8.50-8.59 (m, 3H), 7.93 (d, J=6.8 Hz, 1H), 7.41 (d, J=6.8 Hz, 1H), 7.23 (s, 1H), 6.90 (dd, J=12.8, 2.0 Hz, 1H), 6.41 (s, 1H), 6.31 (t, J=7.2 Hz, 1H), 4.97-5.00 (m, 3H), 4.47-4.48 (m, 2H), 4.10-4.11 (m, 1H), 3.47-3.60 (m, 3H), 2.98-3.09 (m, 4H), 2.74-2.88 (m, 1H), 2.58-2.68 (m, 2H), 1.93-2.21 (m, 10H), 1.45-1.52 (m, 8H), 1.26-1.29 (m, 2H). LCMS [ES⁺]: m/z 787.65 [M+H]⁺

### Example 137 was Prepared Substantially Following the Synthesis of Example 113

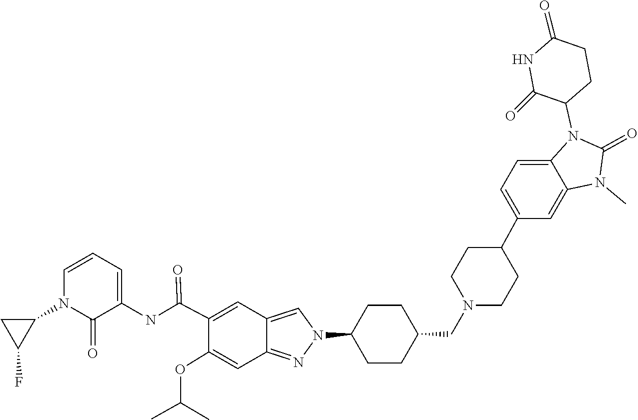

### 2-((1r,4S)-4-((4-(1-(2,6-dioxopiperidin-3-yl)-3-methyl-2-oxo-2,3-dihydro-1H-benzo[d]imidazol-5-yl)piperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H-NMR (400 MHz, DMSO-d₆): δ 11.10 (s, 1H), 10.88 (s, 1H), 8.84 (br s, 1H), 8.59 (s, 1H), 8.56 (s, 1H), 8.52 (dd, J=7.4, 1.4 Hz, 1H), 7.41 (d, J=7.2 Hz, 1H), 7.23 (s, 1H), 7.04-7.09 (m, 2H), 6.93 (d, J=8.4 Hz, 1H), 6.31 (t, J=7.2 Hz, 1H), 5.34-5.39 (m, 1H), 5.01-5.20 (m, 1H), 4.50 (br s, 1H), 3.66 (d, J=11.6 Hz, 2H), 3.35-3.46 (m, 4H), 3.02-3.15 (m, 4H), 2.87-2.91 (m, 2H), 2.54-2.68 (m, 3H), 2.20-2.23 (m, 2H), 1.75-2.15 (m, 10H), 1.35-1.70 (m, 8H), 1.20-1.34 (m, 2H). LCMS (ES⁺): m/z 807.77 [M+H]⁺

### Example 138 was Prepared Substantially Following the Synthesis of Example 113

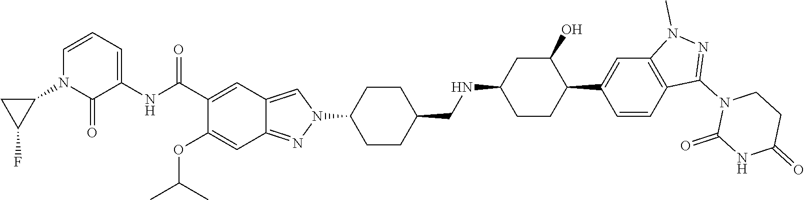 [SMILES: CC(C)Oc1cc2nn([C@H]3CC[C@H](CN[C@@H]4CC[C@H](c5ccc6c(N7CCC(=O)NC7=O)nn(C)c6c5)[C@H](O)C4)CC3)cc2cc1C(=O)Nc1cccn([C@H]2C[C@H]2F)c1=O]

### 2-((1R,4S)-4-((((1R,3R,4R)-4-(3-(2,4-dioxotetrahydropyrimidin-1(2H)-yl)-1-methyl-1H-indazol-6-yl)-3-hydroxycyclohexyl)amino)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H-NMR (400 MHz, DMSO-d₆) δ 10.98 (s, 1H), 10.55 (s, 1H), 8.58 (s, 2H), 8.51 (dd, J=7.4, 1.4 Hz, 1H), 8.23 (br s, 1H), 7.94-7.92 (m, 1H), 7.57 (d, J=8.4 Hz, 1H), 7.41 (d, J=6.8 Hz, 2H), 7.16 (s, 1H), 6.32 (t, J=7.2 Hz, 1H), 5.23 (s, 1H), 5.01-4.96 (m, 2H), 4.48 (br s, 1H), 4.13 (s, 1H), 3.97 (s, 3H), 3.91 (t, J=6.6 Hz, 2H), 3.65-3.46 (m, 2H), 3.01-2.97 (m, 3H), 2.76 (t, J=6.8 Hz, 2H), 2.54 (s, 3H), 2.54-2.50 (m, 1H), 2.26-2.20 (m, 5H), 1.97-1.91 (m, 5H), 1.88-1.82 (m, 1H), 1.67-1.65 (m, 2H), 1.51 (d, J=3.6 Hz, 6H), 1.29-1.24 (m, 2H). LCMS (ES⁺): m/z 822.88 [M+H]⁺

### Example 139 was Prepared Substantially Following the Synthesis of Example 113

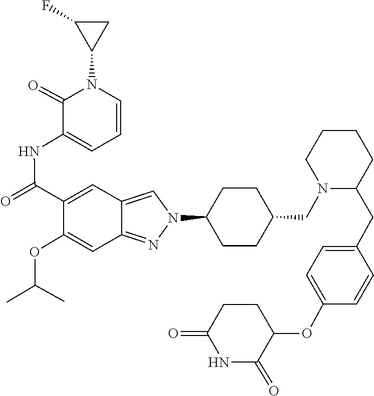 [SMILES: CC(C)Oc1cc2nn([C@H]3CC[C@H](CN4CCCCC4Cc4ccc(OC5CCC(=O)NC5=O)cc4)CC3)cc2cc1C(=O)Nc1cccn([C@H]2C[C@H]2F)c1=O]

### 2-((1r,4S)-4-((2-(4-((2,6-dioxopiperidin-3-yl)oxy)benzyl)piperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆) δ 10.88 (d, J=4.4 Hz, 2H),8.56 (d, J=10.0 Hz, 1H), 8.52 (dd, J=7.4, 1.4 Hz, 2H), 7.41 (d, J=0.8 Hz, 1H), 7.24 (s, 1H), 7.11 (d, J=8.4 Hz, 2H), 6.93 (d, J=8.4 Hz, 2H), 6.32 (t, J=7.2 Hz, 1H), 5.13-4.98 (m, 3H), 4.56-4.52 (m, 1H), 3.46-3.12 (m, 1H), 3.02-2.97 (m, 2H), 2.76-2.54 (m, 5H), 2.39-2.14 (m, 6H), 2.12-2.09 (m, 4H), 1.60-1.41 (m, 13H), 1.41-1.10 (m, 4H). LCMS (ES⁺): m/z 766.96 [M+H]⁺

### Example 140 was Prepared Substantially Following the Synthesis of Example 113

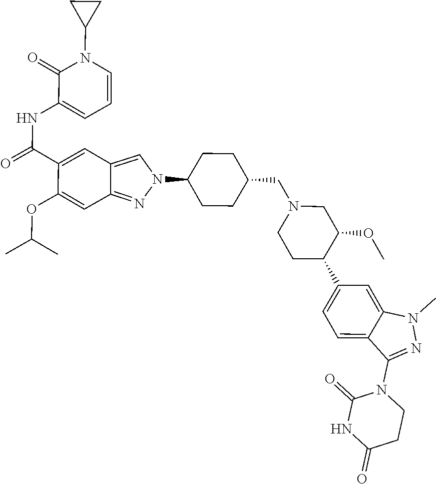 [SMILES: CO[C@H]1CN(C[C@H]2CC[C@H](n3cc4cc(C(=O)Nc5cccn(C6CC6)c5=O)c(OC(C)C)cc4n3)CC2)CC[C@H]1c1ccc2c(N3CCC(=O)NC3=O)nn(C)c2c1]

### N-(1-cyclopropyl-2-oxo-1,2-dihydropyridin-3-yl)-2-((1R,4r)-4-(((3R,4S)-4-(3-(2,4-dioxotetrahydropyrimidin-1(2H)-yl)-1-methyl-1H-indazol-6-yl)-3-methoxypiperidin-1-yl)methyl)cyclohexyl)-6-isopropoxy-2H-indazole-5-carboxamide

¹H-NMR (400 MHz, DMSO-d₆) δ 10.89 (s, 1H), 10.53 (s, 1H), 8.56 (d, J=6.0 Hz, 2H), 8.48 (dd, J=7.6, 1.6 Hz, 1H), 7.53 (d, J=8.4 Hz, 1H), 7.43 (s, 1H), 7.28 (dd, J=7.2, 1.6 Hz, 1H), 7.24 (s, 1H), 7.11 (d, J=8.8 Hz, 1H), 6.25 (t, J=7.2 Hz, 1H), 4.99-4.98 (m, 1H), 4.45-4.38 (m, 1H), 3.97 (s, 3H), 3.90 (t, J=6.6 Hz, 2H), 3.54-3.47 (m, 2H), 3.32 (s, 1H), 3.06 (s, 4H), 2.82-2.80 (m, 1H), 2.74 (t, J=6.8 Hz, 2H), 2.20-1.92 (m, 11H), 1.64-1.53 (m, 2H), 1.52 (d, J=6.0 Hz, 6H), 1.12-1.02 (m, 4H), 0.92-0.90 (m, 2H). LCMS (ES⁺): m/z 804.72 [M+H]⁺

### Example 141 was Prepared Substantially Following the Synthesis of Example 113

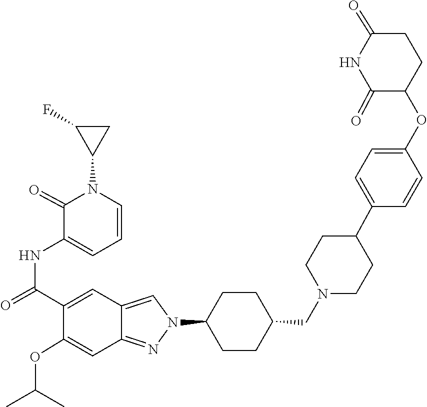 [SMILES: CC(C)Oc1cc2nn([C@H]3CC[C@H](CN4CCC(c5ccc(OC6CCC(=O)NC6=O)cc5)CC4)CC3)cc2cc1C(=O)Nc1cccn([C@H]2C[C@H]2F)c1=O]

### 2-((1r,4S)-4-((4-(4-((2,6-dioxopiperidin-3-yl)oxy)phenyl)piperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H-NMR (400 MHz, DMSO-d₆): δ 10.93 (s, 1H), 10.88 (s, 1H), 8.88 (br s, 1H), 8.60 (d, J=4.4 Hz, 1H), 8.55 (s, 1H), 8.52 (dd, J=7.4, 1.4 Hz, 1H), 7.41 (d, J=7.2 Hz, 1H), 7.30-7.16 (m, 3H), 7.00 (d, J=8.4 Hz, 2H), 6.31 (t, J=7.2 Hz, 1H), 5.15-4.97 (m, 3H), 4.49 (br s, 1H), 3.64 (d, J=11.2 Hz, 2H), 3.30-2.95 (m, 5H), 2.72-2.58 (m, 4H), 2.22-2.14 (m, 4H), 2.10 (br s, 8H), 1.70-1.40 (m, 8H), 1.30-1.24 (m, 2H). LCMS (ES⁺): m/z 753.79 [M+H]⁺

### Synthesis of Example 142 N-(1-cyclopropyl-2-oxo-1,2-dihydropyridin-3-yl)-2-((1S,4r)-4-((4-(4-(((S)-2,6-dioxopiperidin-3-yl)oxy)phenyl)piperidin-1-yl)methyl)cyclohexyl)-6-isopropoxy-2H-indazole-5-carboxamide and Example 143 N-(1-cyclopropyl-2-oxo-1,2-dihydropyridin-3-yl)-2-((1R,4r)-4-((4-(4-(((R)-2,6-dioxopiperidin-3-yl)oxy)phenyl)piperidin-1-yl)methyl)cyclohexyl)-6-isopropoxy-2H-indazole-5-carboxamide

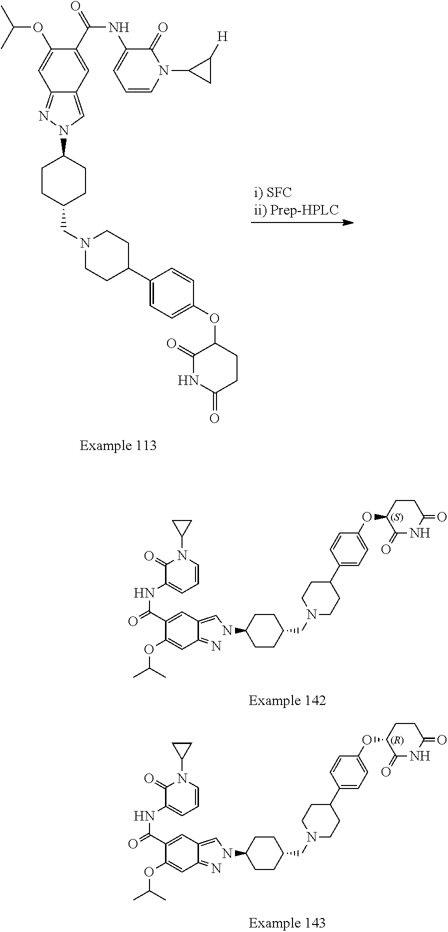

Example 113 (0.200 g) was separated by SFC to obtain single stereoisomer. During SFC separation, fractions of were collected in TFA buffer to avoid glutarimide ring-opening, as the SFC separation method involved use of basic additive. The absolute configuration of both stereoisomers was not determined. The early eluting peak from SFC (Example 142) was arbitrarily assigned as the S-isomer, and the late eluting peak (Example 143) was arbitrarily assigned as the R-isomer. The fractions from SFC were further purified by prep-HPLC to remove the ammonium trifluoro acetate salt.

### Preparative SFC Conditions:

Column/dimensions: R, R-WHELK-01 (30×250) mm, 5μ

% CO: 50%

% Co solvent: 50% (0.2% 7N Methanolic AMMONIA IN

ACN: IPA) (1:1)

Total Flow: 120 g/min

Back Pressure: 00 bar

Temperature: 30° C.

UV:330 nm

Solubility: MeOH+THF+ACN

Prep. HPLC Condition:

Column/dimensions: SUNFIRE C18 (19*150, 5 um)

Mobile phase A: 0.05% TFA in water

Mobile phase B: 100% ACN

Gradient (Time/% B):0/15,2/15,13/39.2,13.1/98,16/98,16.1/15,18/15

Flow rate: 17 ml/min

Solubility: THF+ACN+H2O

### Example 142

LCMS (ES⁺): m/z 735,74 [M+H]

¹H-NMR (400 MHz, DMSO-d₆) δ 10.93-10.89 (m, 2H), 8.83 (br s, 1H), 8.59-8.55 (m, 2H), 8.48 (dd, J=7.4, 1.4 Hz, 1H), 7.29 (dd, J=7.0, 1.4 Hz, 1H), 7.23-7.21 (m, 1H), 7.17 (d, J=8.8 Hz, 1H), 7.09-6.96 (m, 2H), 6.26 (t, J=7.2 Hz, 1H), 5.19-5.15 (m, 1H), 5.01-4.98 (m, 1H), 4.42-4.52 (m, 1H), 3.65-3.50 (m, 2H), 3.49-3.46 (m, 1H), 2.95-3.05 (m, 4H), 2.72-2.69 (m, 1H), 2.62 (d, J=4.4 Hz, 2H), 2.22-2.14 (m, 4H), 1.97-1.91 (m, 9H), 1.52 (d, J=6.0 Hz, 6H), 1.30-1.24 (m, 3H), 1.05-1.04 (m, 2H), 0.91-0.90 (m, 2H).

### Example 143

LCMS (ES⁺): m/z 735.74 [M+H]

¹H-NMR (400 MHz, DMSO-d₆) δ 10.93-10.89 (m, 2H), 8.82 (br s, 1H), 8.59 (s, 1H), 8.55 (s, 1H), 8.48 (dd, J=7.6, 1.6 Hz, 1H), 7.29 (dd, J=7.0, 1.8 Hz, 1H), 7.23 (s, 1H), 7.17 (d, J=8.8 Hz, 1H), 7.01-6.96 (m, 3H), 6.26 (t, J=7.2 Hz, 1H), 5.19-5.15 (m, 1H), 5.01-4.98 (m, 1H), 4.50 (br s, 1H), 3.66-3.63 (m, 2H), 3.51-3.46 (m, 1H), 3.06 (br s, 4H), 2.72-2.67 (m, 1H), 2.63-2.51 (m, 2H), 2.22-2.14 (m, 4H), 2.15-1.91 (m, 9H), 1.52 (d, J=6.0 Hz, 6H), 1.30-1.27 (m, 2H), 1.05-1.04 (m, 2H), 0.92-0.90 (m, 2H).

### Example 144 was Prepared Substantially Following the Synthesis of Example 47

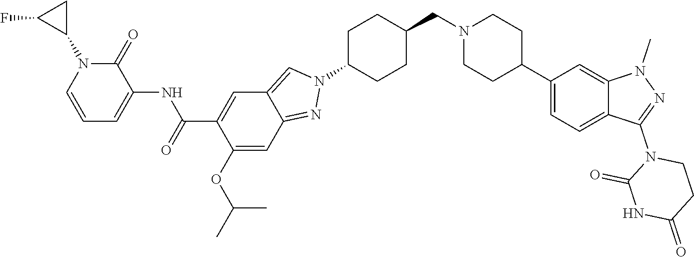 [SMILES: CC(C)Oc1cc2nn([C@H]3CC[C@H](CN4CCC(c5ccc6c(N7CCC(=O)NC7=O)nn(C)c6c5)CC4)CC3)cc2cc1C(=O)Nc1cccn([C@H]2C[C@H]2F)c1=O]

### 2-((1r,4S)-4-((4-(3-(2,4-dioxotetrahydropyrimidin-1(2H)-yl)-1-methyl-1H-indazol-6-yl)piperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H-NMR (400 MHz, DMSO-d₆) δ 10.91 (s, 1H), 8.54 (dd, J=18.2, 7.4 Hz, 2H), 8.17 (s, 1H), 7.55 (d, J=8.8 Hz, 1H), 7.47 (s, 1H), 7.40 (d, J=6.4 Hz, 1H), 7.25 (s, 1H), 7.05 (d, J=8.4 Hz, 1H), 6.32 (t, J=7.2 Hz, 1H), 4.99 (dd, J=11.6, 5.6 Hz, 2H), 4.44 (s, 1H), 3.97-3.89 (m, 6H), 3.46 (s, 1H), 3.33-3.02 (m, 2H), 3.33-3.02 (m, 3H), 2.23-2.03 (m, 4H), 2.03-1.59 (m, 12H), 1.51 (dd, J=5.6, 2.0 Hz, 7H), 1.14 (d, J=11.2 Hz, 2H). LCMS (ES⁺): m/z 792.64[M+H]⁺

### Example 145 was Prepared Substantially Following the Synthesis of Example 47

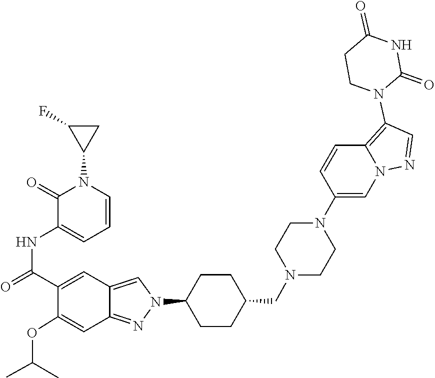 [SMILES: CC(C)Oc1cc2nn([C@H]3CC[C@H](CN4CCN(c5ccc6c(N7CCC(=O)NC7=O)cnn6c5)CC4)CC3)cc2cc1C(=O)Nc1cccn([C@H]2C[C@H]2F)c1=O]

### 2-((1r,4S)-4-((4-(3-(2,4-dioxotetrahydropyrimidin-1(2H)-yl)pyrazolo[1,5-a]pyridin-6-yl)piperazin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H-NMR (400 MHz, DMSO-d₆): δ 10.90 (S, 1H), 10.40 (s, 1H), 8.57 (s, 1H), 8.55 (s, 1H), 8.52 (d, J=6.4 Hz, 1H), 8.00 (s, 1H), 7.85 (s, 1H), 7.47 (d, J=9.6 Hz, 1H), 7.40 (d, J=6.0 Hz, 1H), 7.28 (d, J=8.4 Hz, 1H), 7.25 (s, 1H), 6.30 (t, J=7.2 Hz, 1H), 5.13-4.97 (m, 2H), 4.46-4.44 (m, 1H), 3.74 (t, J=6.6 Hz, 2H), 3.48-3.46 (m, 1H), 3.32-3.12 (m, 4H), 2.78-2.74 (m, 2H), 2.55-2.50 (m, 4H), 2.28-2.12 (m, 4H), 2.02-1.89 (m, 4H), 1.70-1.47 (m, 9H), 1.21-1.11 (m, 2H). LCMS (ES⁺): m/z 779.57 [M+H]⁺

### Example 146 was Prepared Substantially Following the Synthesis of Example 47

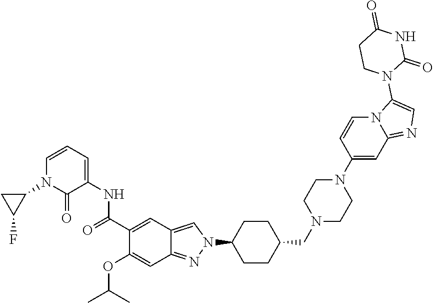 [SMILES: CC(C)Oc1cc2nn([C@H]3CC[C@H](CN4CCN(c5ccn6c(N7CCC(=O)NC7=O)cnc6c5)CC4)CC3)cc2cc1C(=O)Nc1cccn([C@H]2C[C@H]2F)c1=O]

### 2-((1r,4S)-4-((4-(3-(2,4-dioxotetrahydropyrimidin-1(2H)-yl)imidazo[1,2-a]pyridin-7-yl)piperazin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H-NMR (400 MHz, DMSO-d₆): δ 10.60 (S, 1H), 10.40 (s, 1H), 8.57 (s, 1H), 8.55 (s, 1H), 8.52 (d, J=6.4 Hz, 1H), 8.15 (s, 1H), 8.04 (d, J=7.6 Hz, 1H), 7.40 (d, J=6.8 Hz, 1H), 7.28 (s, 1H), 7.25 (s, 1H), 6.89 (d, J=6.8 Hz, 1H), 6.68 (s, 1H), 6.32 (t, J=7.2 Hz, 1H), 5.14-4.94 (m, 2H), 4.46-4.44 (m, 1H), 3.75 (t, J=6.6 Hz, 2H), 3.48-3.46 (m, 1H), 3.32-3.12 (m, 4H), 2.78-2.74 (m, 2H), 2.55-2.50 (m, 4H), 2.28-2.12 (m, 4H), 2.02-1.89 (m, 4H), 1.74-1.39 (m, 9H), 1.23-1.08 (m, 2H). LCMS (ES⁺): m/z 779.57 [M+H]⁺

### Example 147 was Prepared Substantially Following the Synthesis of Example 62

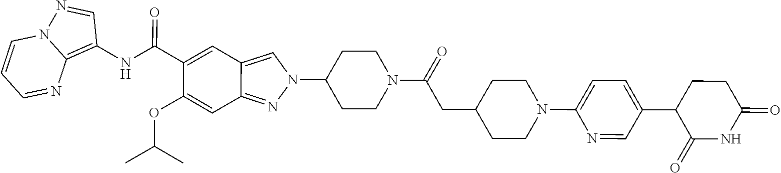 [SMILES: CC(C)Oc1cc2nn(C3CCN(C(=O)CC4CCN(c5ccc(C6CCC(=O)NC6=O)cn5)CC4)CC3)cc2cc1C(=O)Nc1cnn2cccnc12]

### 2-(1-(2-(1-(5-(2,6-dioxopiperidin-3-yl)pyridin-2-yl)piperidin-4-yl)acetyl)piperidin-4-yl)-6-isopropoxy-N-(pyrazolo[1,5-a]pyrimidin-3-yl)-2H-indazole-5-carboxamide

¹H NMR (400 MHz, DMSO-d₆): δ: 10.80 (s, 1H), 10.75 (s, 1H), 9.07 (q, 1H), 8.80 (s, 1H), 8.62 (d, J=3.3 Hz, 2H), 8.53 (q, 1H), 7.94 (d, J=2.4 Hz, 1H), 7.36 (q, 1H), 7.29 (s, 1H), 7.04 (q, 1H), 6.80 (d, J=8.8 Hz, 1H), 5.04 (m, 1H), 4.76 (m, 1H), 4.57 (m, 1H), 4.26 (m, 2H), 4.09 (m, 1H), 3.72 (m, 1H), 3.22 (m, 1H), 2.80 (m, 3H), 2.66 (m, 1H), 2.53 (m, 1H), 2.34 (m, 2H), 2.16 (m, 3H), 1.96 (m, 4H), 1.75 (m, 2H), 1.55 (m, 6H), 1.19 (m, 2H). LCMS (ES⁺): m/z 733.11 [M+H]⁺

### Example 148 was Prepared Substantially Following the Synthesis of Example 62

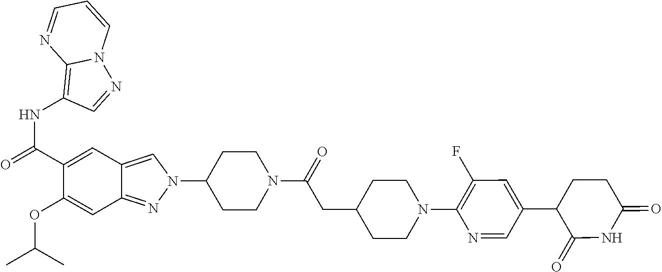

### 2-(1-(2-(1-(5-(2,6-dioxopiperidin-3-yl)-3-fluoropyridin-2-yl)piperidin-4-yl)acetyl)piperidin-4-yl)-6-isopropoxy-N-(pyrazolo[1,5-a]pyrimidin-3-yl)-2H-indazole-5-carboxamide

¹H NMR (400 MHz, DMSO-d₆): δ: 10.85 (s, 1H), 10.75 (s, 1H), 9.07 (q, 1H), 8.80 (s, 1H), 8.62 (d, J=2.2 Hz, 2H), 8.53 (q, 1H), 7.87 (s, 1H), 7.43 (q, 1H), 7.30 (s, 1H), 7.04 (q, 1H), 5.04 (m, 1H), 4.76 (m, 1H), 4.57 (m, 1H), 4.10 (m, 1H), 3.89 (m, 2H), 3.24 (m, 1H), 2.83 (m, 4H), 2.69 (m, 1H), 2.55 (m, 1H), 2.35 (m, 2H), 2.20 (m, 3H), 1.98 (m, 4H), 1.78 (m, 2H), 1.55 (m, 6H), 1.30 (m, 2H). LCMS (ES⁺): m/z 751.17 [M+H]⁺

### Example 149 was Prepared Substantially Following the Synthesis of Example 62

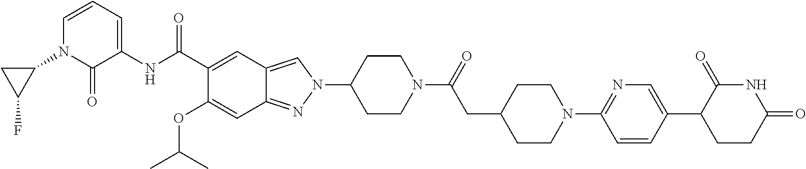 [SMILES: CC(C)Oc1cc2nn(C3CCN(C(=O)CC4CCN(c5ccc(C6CCC(=O)NC6=O)cn5)CC4)CC3)cc2cc1C(=O)Nc1cccn([C@H]2C[C@H]2F)c1=O]

### 2-(1-(2-(1-(5-(2,6-dioxopiperidin-3-yl)pyridin-2-yl)piperidin-4-yl)acetyl)piperidin-4-yl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 10.80 (s, 1H), 10.79 (s, 1H), 8.57 (d, J=4.8 Hz, 2H), 8.52 (d, J=7.6 Hz, 1H), 7.94 (s, 1H), 7.40 (d, J=8.4 Hz, 1H), 7.36 (d, J=8.8 Hz, 1H), 7.26 (s, 1H), 6.80 (d, J=8.8 Hz, 1H), 6.32 (d, J=7.2 Hz, 1H), 5.17-4.92 (m, 2H), 4.83-4.76 (m, 1H), 4.59 (d, J=8.8 Hz, 1H), 4.34-4.26 (d, J=9.2 Hz, 2H), 4.09 (d, J=7.2 Hz, 1H), 3.76-3.68 (m, 1H), 3.49 (m, 1H), 3.33 (bs, 1H), 2.84-2.56 (m, 5H), 2.36 (m, 2H), 2.23-2.11 (m, 3H), 2.04-1.86 (m, 4H), 1.82-1.56 (m, 4H), 1.52 (d, J=6.0 Hz, 6H), 1.49-1.38 (m, 1H), 1.28-1.14 (m, 2H). LCMS (ES⁺): m/z 767.39 [M+H]⁺.

### Example 150 was Prepared Substantially Following the Synthesis of Example 62

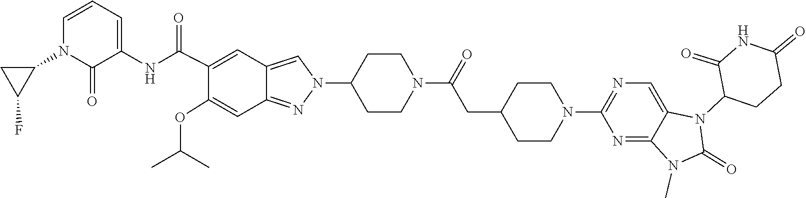 [SMILES: CC(C)Oc1cc2nn(C3CCN(C(=O)CC4CCN(c5ncc6c(n5)n(C)c(=O)n6C5CCC(=O)NC5=O)CC4)CC3)cc2cc1C(=O)Nc1cccn([C@H]2C[C@H]2F)c1=O]

### 2-(1-(2-(1-(7-(2,6-dioxopiperidin-3-yl)-9-methyl-8-oxo-8,9-dihydro-7H-purin-2-yl)piperidin-4-yl)acetyl)piperidin-4-yl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 11.11 (s, 1H), 10.87 (s, 1H), 8.58 (d, J=4.8 Hz, 2H), 8.51 (dd, J=7.4, 1.4 Hz, 1H), 7.99 (s, 1H), 7.40 (d, J=7.2 Hz, 1H), 8.51 (dd, J=7.6, 1.6 Hz, 1H), 6.30 (t, J=7.2 Hz, 1H), 5.32-5.34 (m, 1H), 5.01-5.18 (m, 2H), 4.75 (br s, 1H), 4.57 (d, J=12.4 Hz, 2H), 3.99-4.05 (m, 2H), 3.45-3.99 (m, 1H), 3.24-3.45 (m, 4H), 2.54-2.94 (m, 7H), 2.34 (d, J=6.8 Hz, 2H), 2.16 (br s, 2H), 2.01-2.07 (m, 4H), 1.77 (d, J=11.2 Hz, 2H), 1.50-1.52 (m, 8H), 1.16-1.19 (m, 2H). LCMS (ES⁺): m/z 838.37 [M+H]⁺.

### Example 151 was Prepared Substantially Following the Synthesis of Example 62

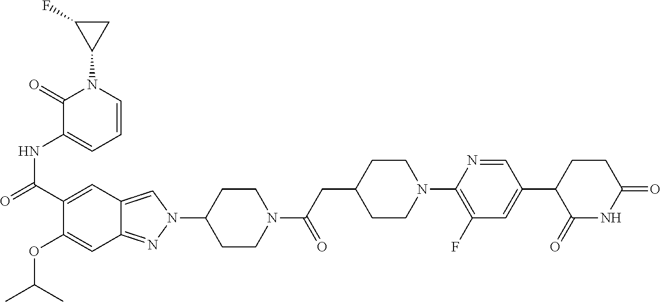 [SMILES: CC(C)Oc1cc2nn(C3CCN(C(=O)CC4CCN(c5ncc(C6CCC(=O)NC6=O)cc5F)CC4)CC3)cc2cc1C(=O)Nc1cccn([C@H]2C[C@H]2F)c1=O]

### 2-(1-(2-(1-(5-(2,6-dioxopiperidin-3-yl)-3-fluoropyridin-2-yl)piperidin-4-yl)acetyl)piperidin-4-yl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 10.85 (s, 2H), 8.59 (d, J=6.0 Hz, 2H), 8.52 (dd, J=7.4, 1.4 Hz, 1H), 7.87 (s, 1H), 7.45 (d, J=2.0 Hz, 1H), 7.40 (d, J=7.2 Hz, 1H), 7.26 (s, 1H), 6.30 (t, J=7.2 Hz, 1H), 5.18-4.95 (m, 2H), 5.0-4.97 (m, 1H), 4.60-4.51 (m, 1H), 3.94-3.92 (m, 1H), 3.90 (d, J=12.4 Hz, 2H), 3.84 (dd, J=12.6, 4.6 Hz, 1H), 3.43 (s, 1H), 3.24 (s, 1H), 2.89-2.79 (m, 4H), 2.55-2.54 (m, 1H), 2.36 (d, J=6.8 Hz, 2H), 2.19-2.16 (m, 3H), 2.01-1.97 (m, 4H), 1.78 (d, J=11.6 Hz, 2H), 1.52-1.50 (m, 1H), 1.51-1.38 (m, 7H), 1.50-1.30 (m, 2H). LCMS (ES⁺): m/z 785.18 [M+H]⁺.

### Example 152 was Prepared Substantially Following the Synthesis of Example 62

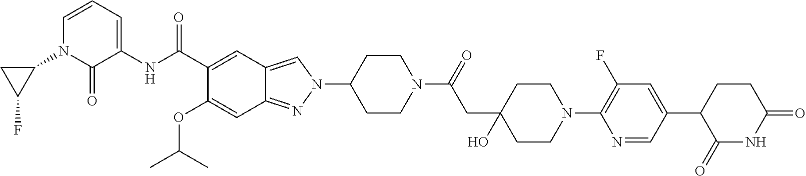 [SMILES: CC(C)Oc1cc2nn(C3CCN(C(=O)CC4(O)CCN(c5ncc(C6CCC(=O)NC6=O)cc5F)CC4)CC3)cc2cc1C(=O)Nc1cccn([C@H]2C[C@H]2F)c1=O]

### 2-(1-(2-(1-(5-(2,6-dioxopiperidin-3-yl)-3-fluoropyridin-2-yl)-4-hydroxypiperidin-4-yl)acetyl)piperidin-4-yl)-N-(1-((S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 11.11 (s, 1H), 10.87 (s, 1H), 8.58 (d, J=4.8 Hz, 2H), 8.51 (dd, J=7.4, 1.4 Hz, 1H), 7.99 (s, 1H), 7.40 (d, J=7.2 Hz, 1H), 8.51 (dd, J=7.6, 1.6 Hz, 1H), 6.30 (t, J=7.2 Hz, 1H), 5.32-5.34 (m, 1H), 5.01-5.18 (m, 2H), 4.75 (br s, 1H), 4.57 (d, J=12.4 Hz, 2H), 3.99-4.05 (m, 2H), 3.45-3.99 (m, 1H), 3.24-3.45 (m, 4H), 2.54-2.94 (m, 7H), 2.34 (d, J=6.8 Hz, 2H), 2.16 (br s, 2H), 2.01-2.07 (m, 4H), 1.77 (d, J=11.2 Hz, 2H), 1.50-1.52 (m, 8H), 1.16-1.19 (m, 2H). LCMS (ES⁺): m/z 838.37 [M+H]⁺.

### Example 153 (5216) was Prepared Substantially Following the Synthesis of Example 62 (5911)

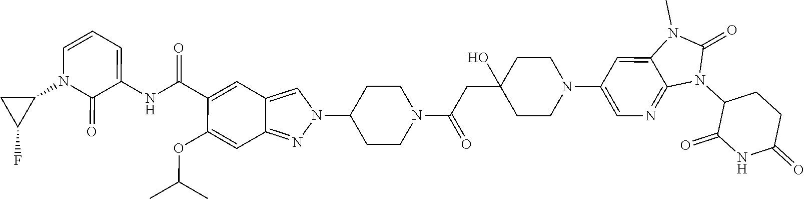 [SMILES: CC(C)Oc1cc2nn(C3CCN(C(=O)CC4(O)CCN(c5cnc6c(c5)n(C)c(=O)n6C5CCC(=O)NC5=O)CC4)CC3)cc2cc1C(=O)Nc1cccn([C@H]2C[C@H]2F)c1=O]

### 2-(1-(2-(1-(3-(2,6-dioxopiperidin-3-yl)-1-methyl-2-oxo-2,3-dihydro-1H-imidazo[4,5-b]pyridin-6-yl)-4-hydroxypiperidin-4-yl)acetyl)piperidin-4-yl)-N-(1-((1S,2R)-2-fluorocyclopropyl)-2-oxo-1,2-dihydropyridin-3-yl)-6-isopropoxy-2H-indazole-5-carboxamide

¹H NMR (400 MHz, DMSO-d₆): δ 11.09 (s, 1H), 10.87 (s, 1H), 8.59 (d, J=2.8 Hz, 2H), 8.52 (dd, J=7.2, 1.6 Hz, 1H), 7.64 (d, J=2.0 Hz, 1H), 7.40 (d, J=7.2 Hz, 1H), 7.34 (d, J=2.0 Hz, 1H), 7.26 (s, 1H), 6.32 (t, J=7.2 Hz, 1H), 5.28-5.32 (m, 1H), 5.01-4.95 (m, 3H), 4.77-4.70 (bs, 1H), 4.69 (d, J=13.6 Hz, 1H), 4.36 (d, J=5.6 Hz, 1H), 3.46-3.42 (m, 1H), 3.35 (s, 5H), 3.32-3.26 (m, 1H), 3.09 (t, J=10.2 Hz, 2H), 2.94-2.81 (m, 3H), 2.64-2.51 (m, 3H), 2.16-2.02 (m, 5H), 1.78-1.69 (m, 5H), 1.52-1.45 (m, 7H). LCMS (ES⁺): m/z 853.19 [M+H]⁺.

### Synthesis of Example 154 2-(1-((1-(5-(2,6-dioxopiperidin-3-yl)-3-fluoropyridin-2-yl)piperidin-4-yl)carbamoyl)piperidin-4-yl)-N-(1-((1S,2R)-2-fluorocyclopropyl)-2-oxo-1,2-dihydropyridin-3-yl)-6-isopropoxy-2H-indazole-5-carboxamide

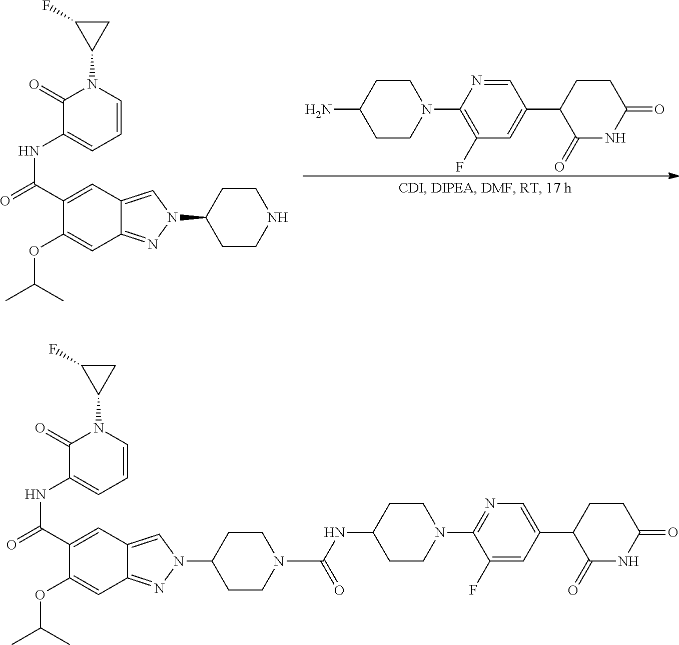

To a stirred solution of N-(1-((1S,2R)-2-fluorocyclopropyl)-2-oxo-1,2-dihydropyridin-3-yl)-6-isopropoxy-2-(piperidin-4-yl)-2H-indazole-5-carboxamide (200 mg, 352.4 μmol, TFA salt) in DMF (3 mL) was added DIPEA (136.64 mg, 1.06 mmol, 184.15 μL) followed by the addition of di(imidazol-1-yl)methanone (68.57 mg, 422.88 μmol) at 0° C. The reaction mixture was stirred at 25° C. for 1 h. Added 3-(6-(4-aminopiperidin-1-yl)-5-fluoropyridin-3-yl)piperidine-2,6-dione (177.76 mg, 422.88 μmol, TFA salt) and the reaction mixture was stirred at 25° C. for 16 h. Upon completion of the reaction, the reaction mixture was concentrated under reduced pressure to get crude product (300 mg). The resulting crude was purified by prep. HPLC to afford 2-(1-((1-(5-(2,6-dioxopiperidin-3-yl)-3-fluoropyridin-2-yl)piperidin-4-yl)carbamoyl)piperidin-4-yl)-N-(1-((1S,2R)-2-fluorocyclopropyl)-2-oxo-1,2-dihydropyridin-3-yl)-6-isopropoxy-2H-indazole-5-carboxamide Example 154 (57.5 mg, 68.18 μmol, 19.35% yield, formic acid salt) as off white solid.

Prep. HPLC condition: Column/dimensions: LUNA OMEGA PS (19*250); Mobile phase A: 0.05% FA in water; Mobile phase B: 100% ACN (Org); Gradient (Time/% B): 0/10,2/25,15.45/66,15.6/98,18/98,18.1/10,20.5/10; Flow rate: 17 mL/min; Solubility: Acetonitrile+THF+Water

¹H NMR (400 MHz, DMSO-d₆): δ 10.89 (s, 2H), 8.58 (d, J=5.6 Hz, 2H), 8.52 (dd, J=7.2, 1.6 Hz, 1H), 7.88 (s, 1H), 7.46 (d, J=1.6 Hz, 1H), 7.41 (dd, J=8.0, 4.4 Hz, 1H), 7.26 (s, 1H), 6.40 (d, J=7.6 Hz, 1H), 6.30 (t, J=7.2 Hz, 1H), 5.00-4.97 (m, 2H), 4.70-4.62 (m, 1H), 4.14 (d, J=12.8 Hz, 2H), 3.98 (d, J=12.8 Hz, 2H), 3.90-3.82 (m, 1H), 3.68-3.62 (m, 1H), 3.46-3.52 (m, 1H), 2.96-2.69 (m, 4H), 2.67-2.66 (m, 1H), 2.55 (s, 1H), 2.32 (t, J=1.6 Hz, 1H), 2.09-2.07 (m, 2H), 1.97-1.81 (m, 5H), 1.51 (d, J=4.0 Hz, 6H), 1.38-1.44 (m, 4H). LCMS (ES⁺): m/z 786.06 [M+H]⁺.

### Synthesis of Example 155 2-(1-((1-(5-(2,6-dioxopiperidin-3-yl)-3-fluoropyridin-2-yl)piperidin-4-yl)(methyl)carbamoyl)piperidin-4-yl)-N-(1-((1S,2R)-2-fluorocyclopropyl)-2-oxo-1,2-dihydropyridin-3-yl)-6-isopropoxy-2H-indazole-5-carboxamide

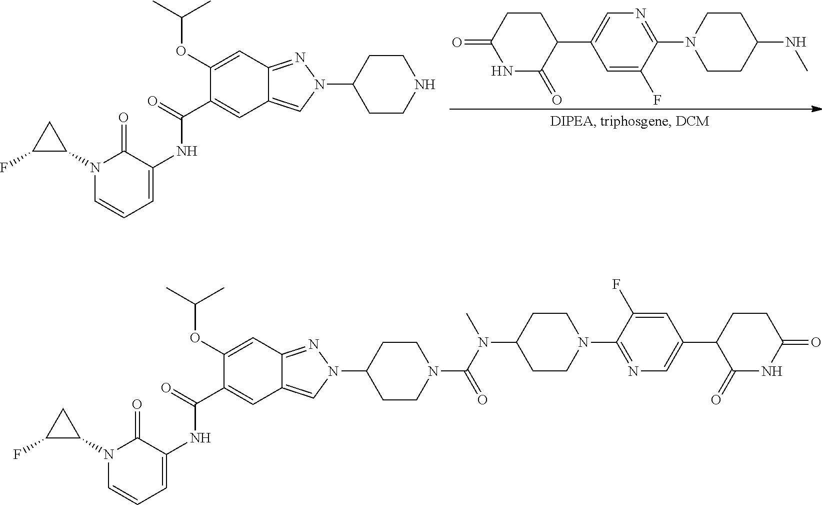 [SMILES: CC(C)Oc1cc2nn(C3CCNCC3)cc2cc1C(=O)Nc1cccn([C@H]2C[C@H]2F)c1=O ; CNC1CCN(c2ncc(C3CCC(=O)NC3=O)cc2F)CC1 ; CC(C)Oc1cc2nn(C3CCN(C(=O)N(C)C4CCN(c5ncc(C6CCC(=O)NC6=O)cc5F)CC4)CC3)cc2cc1C(=O)Nc1cccn([C@H]2C[C@H]2F)c1=O]

In the first round bottom flask, To a stirred solution of 3-[5-fluoro-6-[4-(methylamino)-1-piperidyl]-3-pyridyl]piperidine-2,6-dione (0.20 g, 460.42 μmol, TFA salt) in DCM (3 mL) was added DIPEA (1.19 g, 9.21 mmol, 1.60 mL) at −10° C. To the cold reaction mixture, triphosgene (150.29 mg, 506.46 μmol) was added at −10° C. and the reaction mixture was warmed to room temperature and stirred for 4 h. The reaction mixture was diluted with DCM (50 mL) and washed with water (50 mL) and brine solution (30 mL). The organic layer was dried over sodium sulfate and concentrated in vacuo to get the acyl intermediate.

In the second round bottom flask, To a stirred solution of 6-isopropoxy-N-[2-oxo-1-[rac-(1S,2R)-2-fluorocyclopropyl]-3-pyridyl]-2-(4-piperidyl) indazole-5-carboxamide (208.80 mg, 426.16 μmol, HCl salt) in DCM (3 mL) was added DIPEA (1.19 g, 9.21 mmol, 1.60 mL) at −10° C. and the reaction mixture was stirred for 10 minutes at the same temperature. This reaction mixture was added dropwise to the acyl intermediate in the first round bottom flask at −10° C. The resulting mixture was stirred at room temperature for 16 h. After completion of the reaction, the reaction mixture was filtered and concentrated under reduced pressure. The residue obtained was purified by prep HPLC to afford 2-[1-[[1-[5-(2,6-dioxo-3-piperidyl)-3-fluoro-2-pyridyl]-4-piperidyl]-methyl-carbamoyl]-4-piperidyl]-6-isopropoxy-N-[2-oxo-1-[(1S,2R)-2-fluorocyclopropyl]-3-pyridyl]indazole-5-carboxamide Example 155 (65 mg, 79.40 μmol, 17.25% yield,) as an off white solid.

### Prep HPLC Condition:

Column/dimensions: X-SELECT C18 (19*250, 5 um)

Mobile phase A: 0.1% FA IN WATER

Mobile phase B: Acetonitrile

Gradient (Time/% B):0/40,1/40,9.27/58.4,9.35/100,13/100,13.10/40,15/40.

Flow rate: 17 ml/min

Solubility: ACN+THF

LCMS (ES⁺): m/z 800.65 [M+H]⁺

¹H-NMR (400 MHz, DMSO-d₆): δ 10.86 (s, 2H), 8.59 (d, J=7.2 Hz, 2H), 8.52 (dd, J=7.2, 1.6 Hz, 1H), 7.89 (s, 1H), 7.46 (dd, J=14.4, 2.0 Hz, 1H), 7.40 (d, J=6.0 Hz, 1H), 7.26 (s, 1H), 6.30 (t, J=7.2 Hz, 1H), 4.99-4.97 (m, 2H), 4.64-4.58 (m, 1H), 4.04 (d, J=12.4 Hz, 2H), 3.87-3.85 (m, 2H), 3.46-3.45 (m, 2H), 3.32-2.96 (m, 1H), 2.96-2.87 (m, 4H), 2.75 (s, 3H), 2.56-2.55 (m, 1H), 2.18-2.12 (m, 1H), 2.07-2.06 (m, 4H), 1.72-1.69 (m, 2H), 1.52-1.50 (m, 3H), 1.52 (d, J=6.0 Hz, 6H), 1.47-1.42 (m, 1H), 1.10-0.91 (m, 2H).

### Synthesis of Example 156 1-(5-(2,6-dioxopiperidin-3-yl)-3-fluoropyridin-2-yl)piperidin-4-yl 4-(5-((1-((1S,2R)-2-fluorocyclopropyl)-2-oxo-1,2-dihydropyridin-3-yl)carbamoyl)-6-isopropoxy-2H-indazol-2-yl)piperidine-1-carboxylate

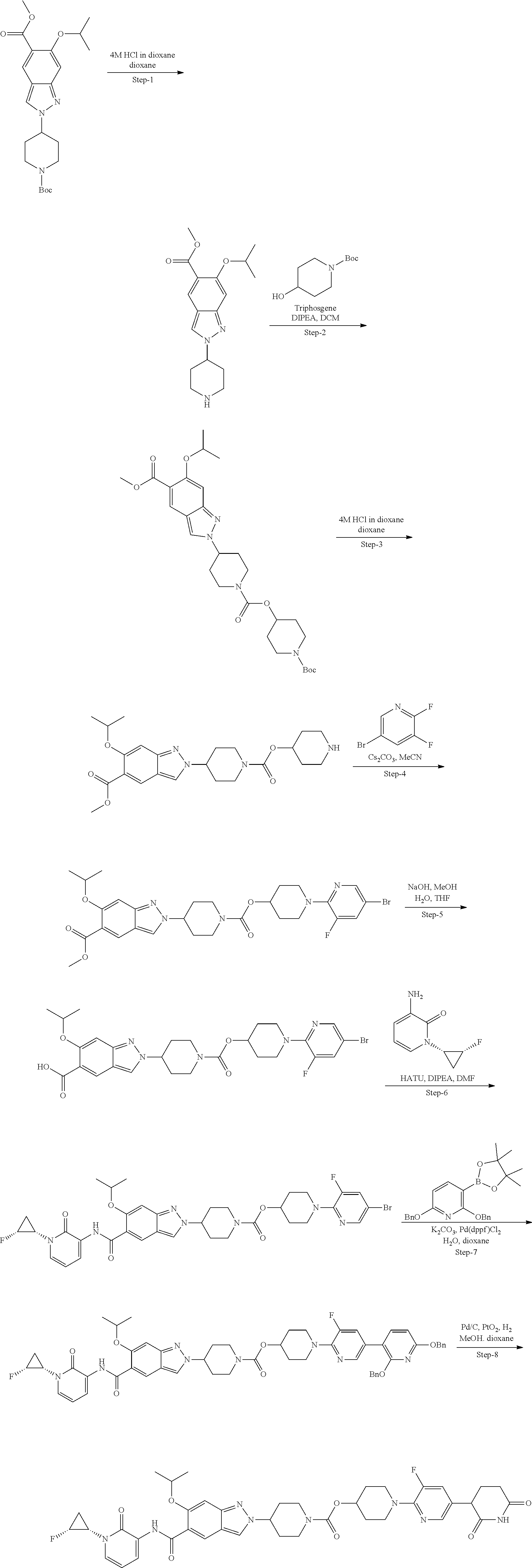

### Step-1:

To a solution of methyl 2-(1-tert-butoxycarbonyl-4-piperidyl)-6-isopropoxy-indazole-5-carboxylate (20 g, 47.90 mmol) in 1,4-dioxane (20 mL) was added 4M HCl in dioxane (20 mL) at 0° C. and the reaction mixture was stirred at 25° C. for 2 h. The reaction mixture was concentrated in vacuo to get the crude product, which was triturated with diethyl ether (200 mL) to afford methyl 6-isopropoxy-2-(4-piperidyl) indazole-5-carboxylate (16.5 g, 45.90 mmol, 95.82% yield, HCl salt) as off white solid. LCMS (ES⁺): m/z 318.35 [M+H]⁺

### Step-2:

To a stirred solution of tert-butyl 4-hydroxypiperidine-1-carboxylate (6 g, 29.81 mmol) in DCM (50 mL) was added DIPEA (6.94 g, 53.66 mmol, 9.35 mL) and stirred for 30 min. Then triphosgene (5.31 g, 17.89 mmol) was added at 0° C. and stirred at the same temperature for 2 h. In a separate flask, methyl 6-isopropoxy-2-(4-piperidyl) indazole-5-carboxylate (10.55 g, 29.81 mmol, HCl salt) in DCM(100 mL) was added DIPEA (9.63 g, 74.53 mmol, 12.98 mL) and stirred for 1 h before the above solution was added slowly at 0° C. and stirred at RT for 4 h. Upon completion of the reaction, the reaction mixture was quenched with water (200 mL) and extracted with DCM(3×40 mL). The combined organic layer was dried over sodium sulfate to give the crude product, which was purified by column chromatography (230-400 mesh silica, 40-50% Ethyl acetate in Hexane) to afford methyl 2-[1-[(1-tert-butoxycarbonyl-4-piperidyl)oxycarbonyl]-4-piperidyl]-6-isopropoxy-indazole-5-carboxylate (5.6 g, 7.63 mmol, 25.59% yield) as pale yellow gum. LCMS (ES⁺): m/z 545.30 [M+H]⁺

### Step-3:

To a solution of methyl 2-[1-[(1-tert-butoxycarbonyl-4-piperidyl)oxycarbonyl]-4-piperidyl]-6-isopropoxy-indazole-5-carboxylate (150 mg, 275.41 μmol) in dioxane (3 mL) was added 4M HCl in dioxane (3 mL) at 0° C. and the reaction mixture was stirred at 25° C. for 2 h. The reaction mixture was concentrated in vacuo to get the crude product, which was triturated with diethyl ether (50 mL) to afford methyl 6-isopropoxy-2-[1-(4-piperidyloxycarbonyl)-4-piperidyl]indazole-5-carboxylate (80 mg, 162.35 μmol, 58.95%

yield, HCl salt) as off white solid. LCMS (ES⁺): m/z 445.51 [M+H]⁺

Step-4: To a stirred solution of methyl 6-isopropoxy-2-[1-(4-piperidyloxycarbonyl)-4-piperidyl]indazole-5-carboxylate (310 mg, 644.51 μmol, HCl salt) in MeCN (8 mL) was added cesium carbonate (629.98 mg, 1.93 mmol) at RT and stirred for 10 min. To the reaction mixture was added 5-bromo-2,3-difluoro-pyridine (150.02 mg, 773.42 μmol) and stirred at 80° C. for 16 h. The reaction mixture was quenched with ice cold water(20 mL) and extracted with ethyl acetate(3×30 mL). The combined organic layer was reduced under pressure to get the crude product, which was purified by column chromatography (230-400 mesh silica, 25-30% ethyl acetate in hexane) to afford methyl 2-[1-[[1-(5-bromo-3-fluoro-2-pyridyl)-4-piperidyl]oxycarbonyl]-4-piperidyl]-6-isopropoxy-indazole-5-carboxylate (176 mg, 243.73 mol, 37.82% yield) as off-white solid. LCMS (ES⁺): m/z 618.58, 620.56 [M, M+2]⁺

### Step-5:

To a stirred solution of methyl 2-[1-[[1-(5-bromo-3-fluoro-2-pyridyl)-4-piperidyl]oxycarbonyl]-4-piperidyl]-6-isopropoxy-indazole-5-carboxylate (1.5 g, 2.43 mmol) in methanol (60 mL), water (30 mL), THF (30 mL) was added sodium hydroxide (194.01 mg, 4.85 mmol) and stirred at RT for 16 h. Upon completion of the reaction, the reaction mixture was concentrated in vacuo and quenched with water (20 mL) and extracted with diethyl ether (3×10 mL). The combined organic layer was washed with brine solution, dried over anhydrous Na₂SO₄, and concentrated under vacuum to give 2-[1-[[1-(5-bromo-3-fluoro-2-pyridyl)-4-piperidyl]oxycarbonyl]-4-piperidyl]-6-isopropoxy-indazole-5-carboxylic acid (1.1 g, 1.69 mmol, 69.78% yield) as off-white solid. LCMS (ES⁺): m/z 604.39, 606.40 [M, M+2]⁺

### Step-6:

To a stirred solution of 2-[1-[[1-(5-bromo-3-fluoro-2-pyridyl)-4-piperidyl]oxycarbonyl]-4-piperidyl]-6-isopropoxy-indazole-5-carboxylic acid (0.250 g, 413.59 μmol) in DMF (3.0 mL) was added DIPEA (106.91 mg, 827.18 μmol, 144.08 L) followed by the addition of 3-amino-1-[(1S,2R)-2-fluorocyclopropyl]pyridin-2-one (84.63 mg, 413.59 μmol, HCl salt) and HATU (314.52 mg, 827.18 μmol). The reaction mixture was stirred at 50° C. for 2 h. Upon completion of the reaction, the reaction mixture was diluted with ice cold water (50 mL), the solid was filtered off to give the crude, which was purified by column chromatography (230-400 mesh silica gel, 0-80% ethyl acetate in pet ether as eluent) to afford [1-(5-bromo-3-fluoro-2-pyridyl)-4-piperidyl] 4-[6-isopropoxy-5-[[2-oxo-1-[(1S,2R)-2-fluorocyclopropyl]-3-pyridyl]carbamoyl]indazol-2-yl]piperidine-1-carboxylate (0.400 g, 395.33 μmol, 31.86% yield) as an off white solid. LCMS (ES⁺): m/z 755.67 [M+H]⁺

### Step-7:

To a stirred solution of [1-(5-bromo-3-fluoro-2-pyridyl)-4-piperidyl] 4-[6-isopropoxy-5-[[2-oxo-1-[(1S,2R)-2-fluorocyclopropyl]-3-pyridyl]carbamoyl]indazol-2-yl]piperidine-1-carboxylate (50 mg, 66.26 μmol) and 2,6-dibenzyloxy-3-(4,4,5,5-tetramethyl-1,3,2-dioxaborolan-2-yl)pyridine (82.95 mg, 198.78 μmol) in water (1 mL) and dioxane (3 mL) was added potassium carbonate (27.47 mg, 198.78 μmol). The reaction mixture was degassed with argon for 10 minutes and Pd(dppf)Cl₂ (2.42 mg, 3.31 μmol) was added. The reaction mixture was degassed with argon for an additional 5 minutes and the reaction mixture was stirred at 90° C. for 16 h. Desired compound was confirmed as [1-[5-(2,6-dibenzyloxy-3-pyridyl)-3-fluoro-2-pyridyl]-4-piperidyl] 4-[6-isopropoxy-5-[[2-oxo-1-[(1S,2R)-2-fluorocyclopropyl]-3-pyridyl]carbamoyl]indazol-2-yl]piperidine-1-carboxylate (50 mg, 22.28 μmol, 33.62% yield).

### Step-8:

To a stirred solution of [1-[5-(2,6-dibenzyloxy-3-pyridyl)-3-fluoro-2-pyridyl]-4-piperidyl] 4-[6-isopropoxy-5-[[2-oxo-1-[(1S,2R)-2-fluorocyclopropyl]-3-pyridyl]carbamoyl]indazol-2-yl]piperidine-1-carboxylate (200.00 mg, 207.24 mol) in methanol (1.5 mL) and dioxane (3.5 mL) was added palladium on carbon (0.100 g, 939.67 μmol) and PtO₂ (0.100 g, 440.37 μmol) The reaction mixture was stirred at 25° C. for 48 h under hydrogen atmosphere at 150 psi pressure. Upon completion of reaction, reaction mixture was filtered through celite bed, washed with 10% methanol in DCM (100 mL), concentrated under reduced pressure to get crude. The crude was purified by prep-HPLC to yield [1-[5-(2,6-dioxo-3-piperidyl)-3-fluoro-2-pyridyl]-4-piperidyl] 4-[6-isopropoxy-5-[[2-oxo-1-[(1S,2R)-2-fluorocyclopropyl]-3-pyridyl]carbamoyl]indazol-2-yl]piperidine-1-carboxylate Example 156 (12.44 mg, 13.52 μmol, 6.52% yield, TFA salt) as a brown solid.

### Prep-HPLC Conditions:

Column/dimensions: LUNA C18

Mobile phase A: 0.1% TFA IN WATER

Mobile phase B: 100% Acetonitrile

Gradient (Time/% B): 0/20,2/20,6/46,15.25/46,15.26/100,24/100,24.01/20,27/20

Flow rate: 18 ml/min

Solubility: Acetonitrile+THF+WATER

¹H-NMR (400 MHz, DMSO-d₆): δ 10.86 (s, 1H), 10.83 (s, 1H), 8.60 (s, 1H), 8.57 (s, 1H), 8.51 (dd, J=7.2, 1.2 Hz, 1H), 7.89 (s, 1H), 7.46 (dd, J=14.4, 1.2 Hz, 1H), 7.40 (d, J=6.0 Hz, 1H), 7.25 (s, 1H), 7.21-6.95 (m, 1H), 6.30 (t, J=7.2 Hz, 1H), 5.18-5.00 (m, 2H), 4.86 (br s, 1H), 4.18-4.15 (m, 2H), 3.86-3.84 (m, 2H), 3.66-3.64 (m, 1H), 3.46-3.45 (m, 1H), 3.35-3.30 (m, 2H), 3.18-2.97 (m, 2H), 2.65-2.58 (m, 2H), 2.33-1.97 (m, 8H), 1.71-1.50 (m, 10H). LCMS (ES⁺): m/z 787.11 [M+H]⁺

### Synthesis of Example 157 2-[1-[[1-[5-(2,6-dioxo-3-piperidyl)-2-pyridyl]-4-piperidyl]carbamoyl]-4-piperidyl]-7-isopropoxy-N-pyrazolo[1,5-a]pyrimidin-3-yl-imidazo[1,2-a]pyridine-6-carboxamide

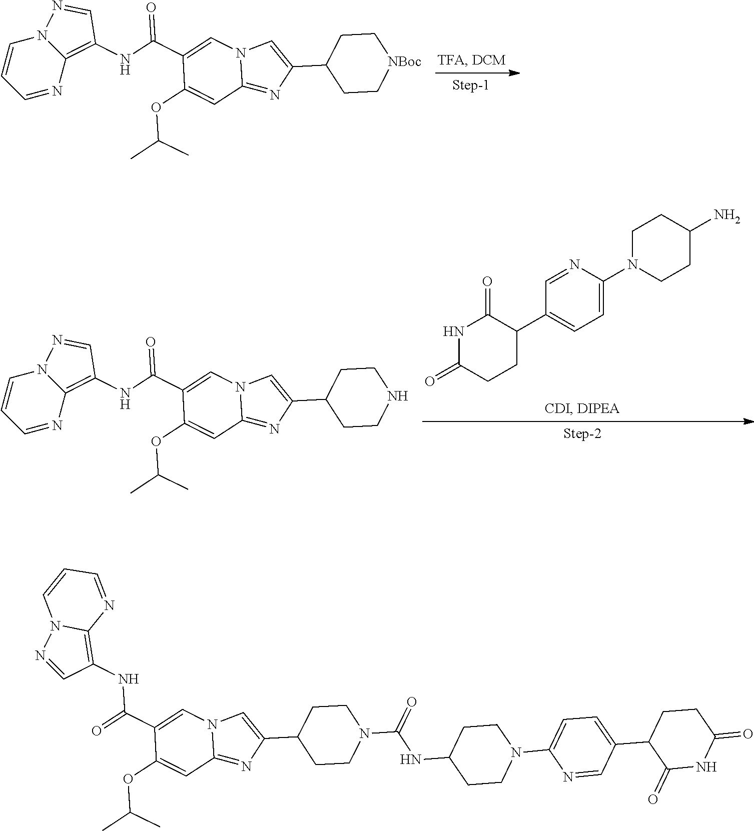

### Step-1:

To a stirred solution of tert-butyl 4-[7-isopropoxy-6-(pyrazolo[1,5-a]pyrimidin-3-ylcarbamoyl)imidazo[1,2-a]pyridin-2-yl]piperidine-1-carboxylate (12.7 g, 24.44 mmol) in DCM (127 mL) was added TFA (38.99 g, 341.95 mmol, 26.34 mL) at 0° C. The reaction mixture was warm to RT and stirred for 16 h. After consumption of the starting material, the solvent was removed to give a residue, which was triturated with diethyl ether to yield a solid precipitate. The diethyl ether layer was decanted and the solid was dried and stirred in THE (100 mL) for 30 minutes. The solid was then filtered and dried to afford 7-isopropoxy-2-(4-piperidyl)-N-pyrazolo[1,5-a]pyrimidin-3-yl-imidazo[1,2-a]pyridine-6-carboxamide (12 g, 21.10 mmol, 86.33% yield, trifluoroacetic acid salt) as a yellow solid. LCMS (ES⁺): m/z 420.33 [M+H]⁺.

### Step-2:

To a stirred solution of 3-[6-(4-amino-1-piperidyl)-3-pyridyl]piperidine-2,6-dione (0.2 g, 693.62 μmol) in a mixture of THE (1 mL) and DMF (1 mL) and DCM (1 mL) was added carbonyldiimidazole (134.96 mg, 832.34 μmol), DIPEA (268.93 mg, 2.08 mmol, 362.44 μL) and the reaction mixture was stirred for 10 min. Compound 7-isopropoxy-2-(4-piperidyl)-N-pyrazolo[1,5-a]pyrimidin-3-yl-imidazo[1,2-a]pyridine-6-carboxamide (148.02 mg, 277.45 mol, TFA salt) was added and the reaction mixture was stirred at room temperature for 16 h. Upon completion of the reaction, the reaction mixture was quenched with water (5 mL) and extracted with ethyl acetate (2×10 mL). The combined organic layer was dried over anhydrous Na₂SO₄, filtered and concentrated under reduced pressure to get the crude product. The crude was purified by Prep-HPLC to afford 2-[1-[[1-[5-(2,6-dioxo-3-piperidyl)-2-pyridyl]-4-piperidyl]carbamoyl]-4-piperidyl]-7-isopropoxy-N-pyrazolo[1,5-a]pyrimidin-3-yl-imidazo[1,2-a]pyridine-6-carboxamide Example 157 (0.042 g, 52.82 μmol, 7.62% yield, formate salt).

Prep-HPLC Condition: Column/dimensions: X BRIDGE C18 (19*250) mm, 5 μm; Mobile phase A: 5 mM AA in water; Mobile phase B: Acetonitrile; Gradient (Time/% B): 0/20,2/20,15/52.2,15.1/98,18/98,18.1/20,21/20; Flow rate: 17 ml/min; Solubility: ACN+WATER.

¹H NMR (400 MHz, DMSO-d₆): δ 10.80 (s, 1H), 10.51 (s, 1H), 9.18 (s, 1H), 9.09 (q, 1H), 8.77 (s, 1H), 8.54 (q, 1H), 7.95 (d, J=2.3 Hz, 1H), 7.74 (s, 1H), 7.37 (q, 1H), 7.18 (s, 1H), 7.06 (q, 1H), 6.83 (d, J=8.8 Hz, 1H), 6.25 (d, J=7.8 Hz, 1H), 5.05 (m, 1H), 4.25 (d, J=11.3 Hz, 1H), 4.02 (d, J=13.0 Hz, 1H), 3.72 (q, 1H), 2.83 (m, 1H), 2.68 (m 1H), 2.18 (m, 1H), 1.95 (m, 1H), 1.76 (d, J=8.6 Hz, 1H), 1.60 (s, 1H), 1.54 (d, J=6.0 Hz, 1H), 1.24 (d, J=5.8 Hz, 1H). LCMS (ES⁺): m/z 734.14 [M+H]⁺.

### Example 158 was Prepared Substantially Following the Synthesis of Example 157

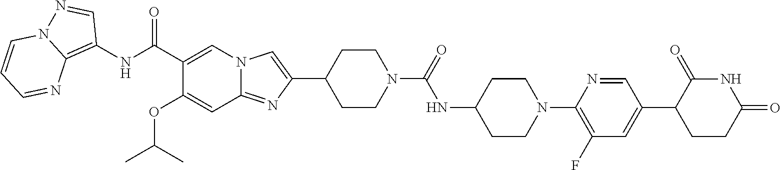 [SMILES: CC(C)Oc1cc2nc(C3CCN(C(=O)NC4CCN(c5ncc(C6CCC(=O)NC6=O)cc5F)CC4)CC3)cn2cc1C(=O)Nc1cnn2cccnc12]

### 2-(1-((1-(5-(2,6-dioxopiperidin-3-yl)-3-fluoropyridin-2-yl)piperidin-4-yl)carbamoyl)piperidin-4-yl)-7-isopropoxy-N-(pyrazolo[1,5-a]pyrimidin-3-yl)imidazo[1,2-a]pyridine-6-carboxamide

¹H NMR (400 MHz, DMSO-d₆): δ 10.85 (s, 1H), 10.51 (s, 1H), 9.18 (s, 1H), 9.09 (q, J=2.9 Hz, 1H), 8.77 (s, 1H), 8.53 (m, 1H), 7.88 (s, 1H), 7.74 (s, 1H), 7.44 (q, 1H), 7.18 (s, 1H), 7.06 (q, 1H), 6.28 (d, J=7.7 Hz, 1H), 5.05 (m, 1H), 4.00 (q, 1H), 3.84 (q, 1H), 3.70 (t, J=3.6 Hz, 1H), 2.86 (m, 1H), 2.69 (m, 1H), 2.55 (d, J=4.9 Hz, 1H), 2.24 (m, 1H), 1.96 (m, 1H), 1.80 (d, J=10.2 Hz, 1H), 1.54 (d, J=6.0 Hz, 1H). LCMS (ES⁺): m/z 752.33 [M+H]⁺.

### Example 159 was Prepared Substantially Following the Synthesis of Example 68

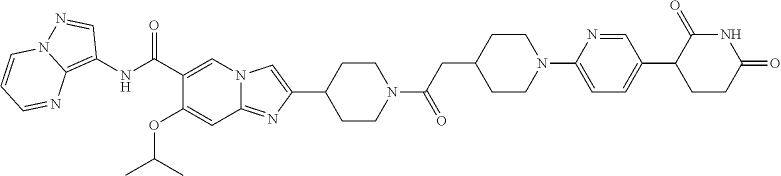 [SMILES: CC(C)Oc1cc2nc(C3CCN(C(=O)CC4CCN(c5ccc(C6CCC(=O)NC6=O)cn5)CC4)CC3)cn2cc1C(=O)Nc1cnn2cccnc12]

### 2-(1-(2-(1-(5-(2,6-dioxopiperidin-3-yl)pyridin-2-yl)piperidin-4-yl)acetyl)piperidin-4-yl)-7-isopropoxy-N-(pyrazolo[1,5-a]pyrimidin-3-yl)imidazo[1,2-a]pyridine-6-carboxamide

¹H NMR (400 MHz, DMSO-d₆): δ: 13.88 (s, 1H), 10.82 (s, 1H), 10.50 (s, 1H), 9.30 (s, 1H), 9.11 (q, 1H), 8.75 (s, 1H), 8.56 (q, 1H), 7.93 (t, J=7.2 Hz, 1H), 7.44 (s, 1H), 7.31 (s, 1H), 7.08 (q, 1H), 6.88 (s, 1H), 5.13 (m, 1H), 4.51 (m, 1H), 4.24 (m, 2H), 4.03 (m, 1H), 3.75 (m, 1H), 3.18-3.15 (m, 2H), 2.77 (m, 2H), 2.54 (m, 3H), 2.32 (m, 3H), 2.19-2.01 (m, 4H), 1.76 (m, 2H), 1.54 (m, 8H), 1.20 (m, 2H). LCMS (ES⁺): m/z 733.15 [M+H]⁺.

### Example 160 was Prepared Substantially Following the Synthesis of Example 68

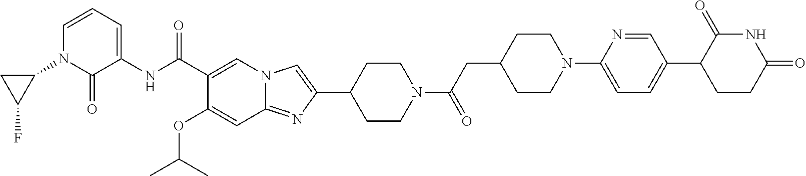 [SMILES: CC(C)Oc1cc2nc(C3CCN(C(=O)CC4CCN(c5ccc(C6CCC(=O)NC6=O)cn5)CC4)CC3)cn2cc1C(=O)Nc1cccn([C@H]2C[C@H]2F)c1=O]

### 2-(1-(2-(1-(5-(2,6-dioxopiperidin-3-yl)pyridin-2-yl)piperidin-4-yl)acetyl)piperidin-4-yl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆): δ: 10.76 (m, 2H), 9.20 (s, 1H), 8.48 (q, 1H), 7.93 (d, J=2.4 Hz, 1H), 7.77 (s, 1H), 7.44 (q, 1H), 7.36 (q, 1H), 7.19 (s, 1H), 6.80 (d, J=9.2 Hz, 1H), 6.34 (t, J=7.2 Hz, 1H), 5.05 (m, 2H), 4.46 (m, 1H), 4.25 (d, J=11.6 Hz, 2H), 3.98 (m, 1H), 3.72 (m, 1H), 3.46 (m, 1H), 3.17 (m, 1H), 2.93 (m, 1H), 2.72 (m, 4H), 2.54 (m, 1H), 2.30 (m, 2H), 2.17 (m, 1H), 1.98 (m, 4H), 1.71-1.24 (m, 14H). LCMS (ES⁺): m/z 767.73 [M+H]⁺.

### Example 161 was Prepared Substantially Following the Synthesis of Example 68

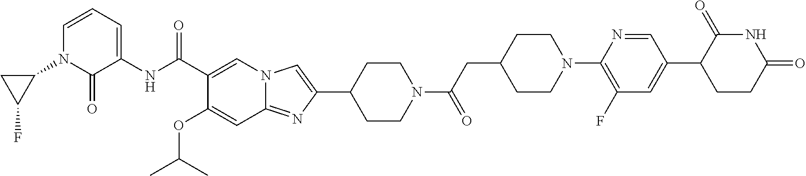 [SMILES: CC(C)Oc1cc2nc(C3CCN(C(=O)CC4CCN(c5ncc(C6CCC(=O)NC6=O)cc5F)CC4)CC3)cn2cc1C(=O)Nc1cccn([C@H]2C[C@H]2F)c1=O]

### 2-(1-(2-(1-(5-(2,6-dioxopiperidin-3-yl)-3-fluoropyridin-2-yl)piperidin-4-yl)acetyl)piperidin-4-yl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆): δ 10.85 (s, 1H), 10.74 (s, 1H), 9.17 (s, 1H), 8.51 (m, 1H), 7.86 (s, 1H), 7.74 (s, 1H), 7.42 (m, 1H), 7.16 (s, 1H), 6.34 (t, J=7.2 Hz, 1H), 5.05 (m, 2H), 4.46 (m, 1H), 3.91 (m, 4H), 3.46 (m, 1H), 3.18 (m, 1H), 2.80-2.54 (m, 6H), 2.27 (m, 3H), 2.00 (m, 4H), 1.75 (m, 2H), 1.55 (m, 11H), 1.29 (m, 2H). LCMS (ES⁻): m/z 783 [M−H]⁻.

### Example 162 was Prepared Substantially Following the Synthesis of Example 68

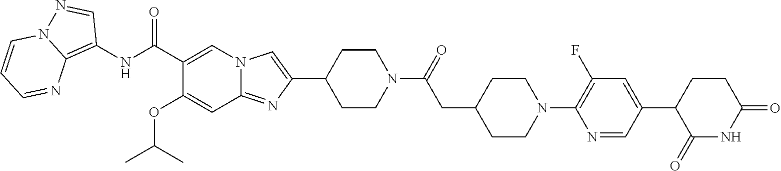 [SMILES: CC(C)Oc1cc2nc(C3CCN(C(=O)CC4CCN(c5ncc(C6CCC(=O)NC6=O)cc5F)CC4)CC3)cn2cc1C(=O)Nc1cnn2cccnc12]

### 2-(1-(2-(1-(5-(2,6-dioxopiperidin-3-yl)-3-fluoropyridin-2-yl)piperidin-4-yl)acetyl)piperidin-4-yl)-7-isopropoxy-N-(pyrazolo[1,5-a]pyrimidin-3-yl)imidazo[1,2-a]pyridine-6-carboxamide

¹H NMR (400 MHz, DMSO-d₆): δ: 10.84 (s, 1H), 10.51 (s, 1H), 9.19 (s, 1H), 9.09 (q, 1H), 8.77 (s, 1H), 8.54 (q, 1H), 8.26 (s, 1H), 7.86 (s, 1H), 7.75 (s, 1H), 7.42 (q, 1H), 7.19 (s, 1H), 7.06 (q, 1H), 5.05 (m, 1H), 4.46 (m, 1H), 3.91 (m, 4H), 3.17 (m, 1H), 2.81 (m, 5H), 2.55 (m, 2H), 2.27 (m, 3H), 1.99 (m, 4H), 1.76 (m, 2H), 1.54 (m, 6H), 1.29 (m, 2H). LCMS (ES⁺): m/z 751.73 [M+H]⁺.

### Example 163 was Prepared Substantially Following the Synthesis of Example 68

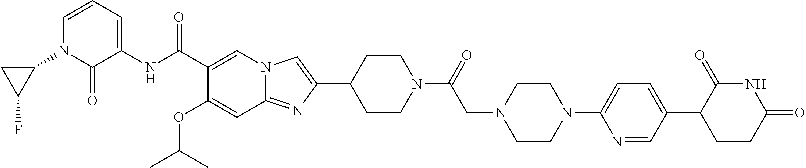 [SMILES: CC(C)Oc1cc2nc(C3CCN(C(=O)CN4CCN(c5ccc(C6CCC(=O)NC6=O)cn5)CC4)CC3)cn2cc1C(=O)Nc1cccn([C@H]2C[C@H]2F)c1=O]

### 2-(1-(2-(4-(5-(2,6-dioxopiperidin-3-yl)pyridin-2-yl)piperazin-1-yl)acetyl)piperidin-4-yl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆) δ: 10.80 (s, 1H), 10.74 (s, 1H), 9.17 (s, 1H), 8.47 (q, 1H), 7.95 (d, J=2.3 Hz, 1H), 7.74 (s, 1H), 7.41 (m, 2H), 7.16 (s, 1H), 6.81 (d, J=8.8 Hz, 1H), 6.33 (t, J=7.2 Hz, 1H), 5.05 (m, 2H), 4.41 (d, J=12.6 Hz, 1H), 4.16 (d, J=12.4 Hz, 1H), 3.74 (q, 1H), 3.45 (d, J=12.7 Hz, 6H), 3.13 (q, 2H), 2.94 (s, 1H), 2.70 (m, 2H), 2.49 (s, 3H), 2.14 (m, 1H), 1.97 (m, 3H), 1.66 (s, 2H), 1.51 (m, 10H). LCMS (ES⁺): m/z 768.22 [M+H]⁺

### Example 164 was Prepared Substantially Following the Synthesis of Example 68

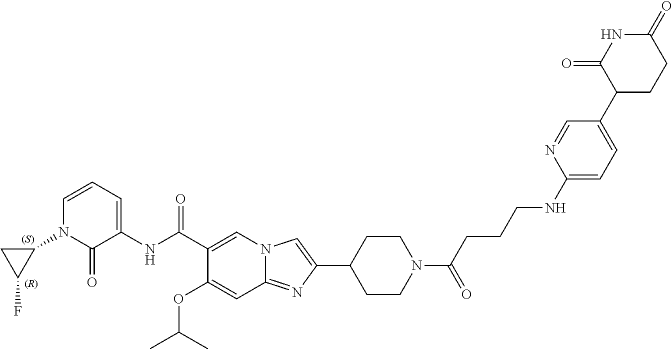

### 2-(1-(4-((5-(2,6-dioxopiperidin-3-yl)pyridin-2-yl)amino)butanoyl)piperidin-4-yl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆) δ: 10.74 (d, 2H), 9.17 (s, 1H), 8.48-8.46 (dd, J=2, 7.6 Hz, 1H), 7.78 (d, J=2 Hz, 1H), 7.72 (s, 1H), 7.43 (d, J=5.6 Hz, 1H), 7.21 (dd, J=2.4, 8.8 Hz, 1H), 7.15 (s, 1H), 6.47 (t, J=5.4 Hz, 1H), 6.42 (d, J=8.4 Hz, 1H), 6.33 (t, J=7.2 Hz, 1H), 5.03-5.01 (m, 2H), 4.44 (d, 1H), 3.92 (d, 1H), 3.65-3.61 (dd, J=4.8, 12 Hz, 1H), 3.46-3.45 (m, 1H), 3.25-3.15 (m, 3H), 2.90 (m, 1H), 2.76-2.59 (m, 2H), 2.49-2.38 (m, 2H), 2.18-1.95 (m, 3H), 1.77-1.74 (m, 2H), 1.68-1.58 (m, 2H), 1.57-1.42 (m, 10H). LCMS (ES⁺): m/z: 727.61 (M+H)⁺

### Example 165 was Prepared Substantially Following the Synthesis of Example 68

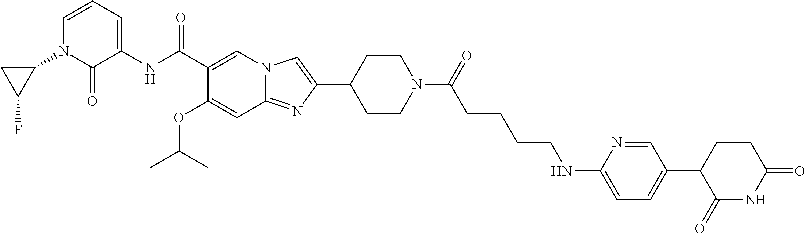 [SMILES: CC(C)Oc1cc2nc(C3CCN(C(=O)CCCCNc4ccc(C5CCC(=O)NC5=O)cn4)CC3)cn2cc1C(=O)Nc1cccn([C@H]2C[C@H]2F)c1=O]

### 2-(1-(5-((5-(2,6-dioxopiperidin-3-yl)pyridin-2-yl)amino)pentanoyl)piperidin-4-yl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆) δ 10.74 (d, 2H), 9.17 (s, 1H), 8.48-8.46 (dd, J=1.6, 7.2 Hz, 1H), 7.78 (d, J=2 Hz, 1H), 7.72 (s, 1H), 7.43 (d, J=6 Hz, 1H), 7.19 (dd, J=2.4, 8.4 Hz, 1H), 7.15 (s, 1H), 6.45-6.40 (m, 2H), 6.33 (t, J=7.2 Hz, 1H), 5.01 (m, 2H), 4.44 (d, 1H), 3.92 (d, 1H), 3.66-3.61 (dd, J=5, 11.8 Hz, 1H), 3.46-3.43 (m, 1H), 3.24-3.15 (m, 3H), 2.90 (m, 1H), 2.79-2.58 (m, 2H), 2.49-32 (m, 4H), 2.19-1.95 (m, 4H), 1.67 (s, 3H), 1.65-1.42 (m, 10H). LCMS (ES⁺): m/z 741.23 [M+H]⁺

### Example 166 was Prepared Substantially Following the Synthesis of Example 47

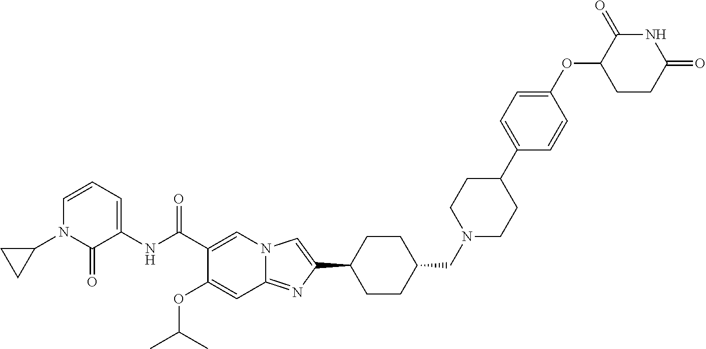 [SMILES: CC(C)Oc1cc2nc([C@H]3CC[C@H](CN4CCC(c5ccc(OC6CCC(=O)NC6=O)cc5)CC4)CC3)cn2cc1C(=O)Nc1cccn(C2CC2)c1=O]

### N-(1-cyclopropyl-2-oxo-1,2-dihydropyridin-3-yl)-2-((1r,4r)-4-((4-(4-((2,6-dioxopiperidin-3-yl)oxy)phenyl)piperidin-1-yl)methyl)cyclohexyl)-7-isopropoxyimidazo[1,2-a]pyridine-6-carboxamide

¹H NMR (400 MHz, DMSO-d₆): δ: 10.91 (s, 1H), 10.76 (s, 1H), 9.17 (s, 1H), 8.45 (m, 1H), 8.14 (s, 1H), 7.72 (d, J=30.7 Hz, 1H), 7.31 (q, J=2.8 Hz, 1H), 7.15 (m, 3H), 6.95 (d, J=8.7 Hz, 2H), 6.27 (m, 1H), 5.15 (m, 1H), 5.03 (m, 1H), 3.49 (m, 1H), 3.11 (m, 2H), 2.71 (m, 6H), 2.59 (m, 1H), 2.15 (m, 5H), 1.91 (d, 2H), 1.72 (m, 5H), 1.52 (m, 8H), 1.05 (m, 3H), 0.90 (m, 2H). LCMS (ES⁺): m/z 735.51 [M+H]⁺.

### Example 167 was Prepared Substantially Following the Synthesis of Example 47

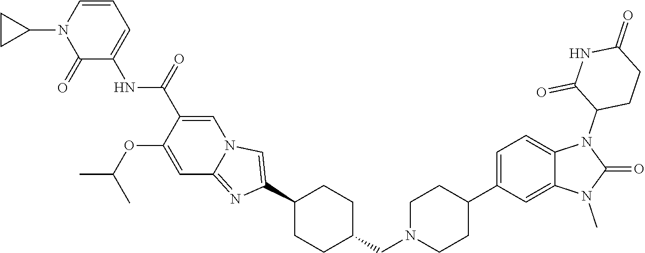 [SMILES: CC(C)Oc1cc2nc([C@H]3CC[C@H](CN4CCC(c5ccc6c(c5)n(C)c(=O)n6C5CCC(=O)NC5=O)CC4)CC3)cn2cc1C(=O)Nc1cccn(C2CC2)c1=O]

### N-(1-cyclopropyl-2-oxo-1,2-dihydropyridin-3-yl)-2-((1r,4r)-4-((4-(1-(2,6-dioxopiperidin-3-yl)-3-methyl-2-oxo-2,3-dihydro-1H-benzo[d]imidazol-5-yl)piperidin-1-yl)methyl)cyclohexyl)-7-isopropoxyimidazo[1,2-a]pyridine-6-carboxamide

¹H NMR (400 MHz, DMSO-d₆): δ: 11.10 (s, 1H), 10.68 (s, 1H), 9.39 (s, 1H), 8.86 (s, 1H), 8.44 (d, J=7.4 Hz, 1H), 7.95 (s, 1H), 7.36 (d, J=6.9 Hz, 2H), 7.21-7.00 (m, 3H), 6.32 (t, J=7.2 Hz, 1H), 5.36 (m, 1H), 5.18 (m, 1H), 3.64 (m, 2H), 3.50 (m, 1H), 3.35 (m, 3H), 2.89 (m, 9H), 2.14-1.79 (m, 11H), 1.52 (m, 7H), 1.21 (m, 1H), 1.05 (m, 2H), 0.91 (m, 2H). LCMS (ES⁺): m/z 789.54 [M+H]⁺.

### Example 168 was Prepared Substantially Following the Synthesis of Example 47

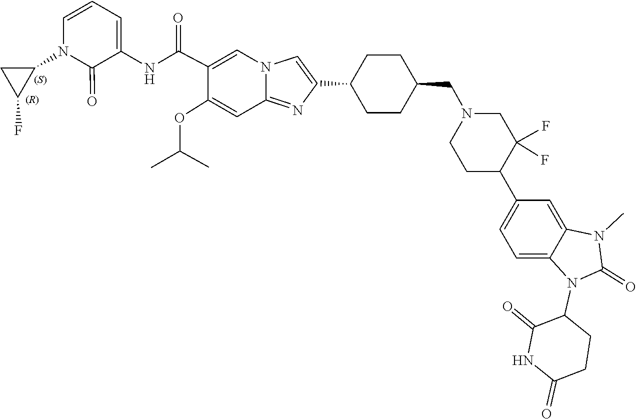

### 2-((1r,4S)-4-((4-(1-(2,6-dioxopiperidin-3-yl)-3-methyl-2-oxo-2,3-dihydro-1H-benzo[d]imidazol-5-yl)-3,3-difluoropiperidin-1-yl)methyl)cyclohexyl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆): δ: 11.10 (s, 1H), 10.75 (s, 1H), 9.17 (d, J=2.2 Hz, 1H), 8.48 (m, J=2.3 Hz, 1H), 7.77 (s, 1H), 7.69 (s, 1H), 7.43-7.07 (m, 4H), 6.34 (t, J=7.2 Hz, 1H), 5.36 (m, 1H), 5.05 (m, 2H), 3.46 (m, 2H), 3.14 (m, 4H), 2.93 (m, 2H), 2.66 (m, 2H), 2.50 (m, 3H), 2.05 (m, 4H), 1.59 (m, 2H), 1.52 (m, 2H), 1.50 (m, 4H) 1.45 (m, 9H), 1.04 (m, 1H). LCMS (ES⁺): m/z 843.73 [M+H]⁺.

### Example 169 was Prepared Substantially Following the Synthesis of Example 47

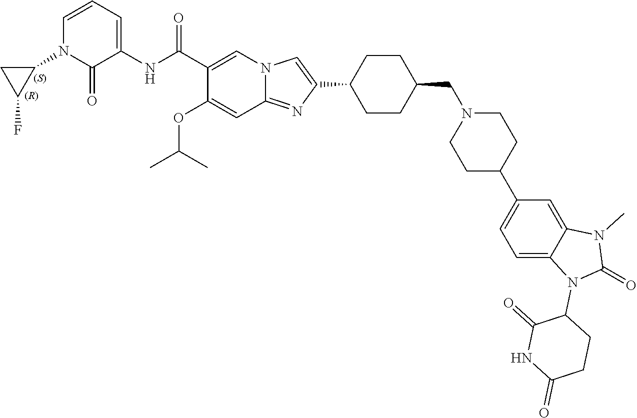

### 2-((1r,4S)-4-((4-(1-(2,6-dioxopiperidin-3-yl)-3-methyl-2-oxo-2,3-dihydro-1H-benzo[d]imidazol-5-yl)piperidin-1-yl)methyl)cyclohexyl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆): δ: 11.10 (s, 1H), 10.63 (s, 1H), 9.47 (d, J=4.8 Hz, 1H), 8.97 (s, 1H), 8.48 (d, J=7.4 Hz, 1H), 8.06 (d, J=21.3 Hz, 1H), 7.48 (m, 2H), 7.07 (m, 2H), 6.93 (m, 1H), 6.38 (t, J=7.2 Hz, 1H), 5.36 (m, 1H), 5.10 (m, 2H), 3.63 (m, 2H), 3.47 (m, 1H), 3.35 (s, 3H), 3.05 (m, 4H), 2.89 (m, 3H), 2.69 (m, 2H), 2.14-1.9 (m, 10H), 1.70-1.57 (m, 9H), 1.49-1.22 (m, 2H). LCMS (ES⁺): m/z 807.40 [M+H]⁺.

### Example 170 was Prepared Substantially Following the Synthesis of Example 47

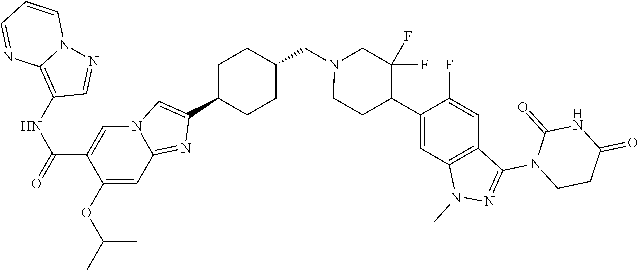 [SMILES: CC(C)Oc1cc2nc([C@H]3CC[C@H](CN4CCC(c5cc6c(cc5F)c(N5CCC(=O)NC5=O)nn6C)C(F)(F)C4)CC3)cn2cc1C(=O)Nc1cnn2cccnc12]

### 2-((1r,4r)-4-((4-(3-(2,4-dioxotetrahydropyrimidin-1(2H)-yl)-5-fluoro-1-methyl-1H-indazol-6-yl)-3,3-difluoropiperidin-1-yl)methyl)cyclohexyl)-7-isopropoxy-N-(pyrazolo[1,5-a]pyrimidin-3-yl)imidazo[1,2-a]pyridine-6-carboxamide

¹H NMR (400 MHz, DMSO-d₆) δ: 10.57 (s, 1H), 10.52 (s, 1H), 9.19 (s, 1H), 9.09 (q, 1H), 8.77 (s, 1H), 8.54 (q, 1H), 7.77 (m, 2H), 7.42 (d, J=10.4 Hz, 1H), 7.17 (s, 1H), 7.06 (m, 1H), 5.05 (m, 1H), 4.03 (s, 3H), 3.92 (m, 2H), 3.52 (m, 1H), 3.20 (m, 1H), 3.03 (m, 1H), 2.75 (m, 2H), 2.62 (m, 1H), 2.34 (m, 7H), 1.93 (m, 2H), 1.84 (m, 1H), 1.54 (m, 9H), 1.07 (m, 2H). LCMS (ES⁺): m/z 812.28 [M+H]⁺

### Example 171 was Prepared Substantially Following the Synthesis of Example 47

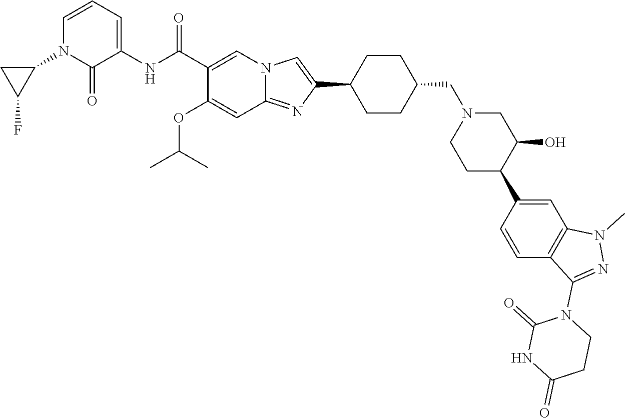 [SMILES: CC(C)Oc1cc2nc([C@H]3CC[C@H](CN4CC[C@H](c5ccc6c(N7CCC(=O)NC7=O)nn(C)c6c5)[C@H](O)C4)CC3)cn2cc1C(=O)Nc1cccn([C@H]2C[C@H]2F)c1=O]

### 2-((1S,4S)-4-(((3S,4R)-4-(3-(2,4-dioxotetrahydropyrimidin-1(2H)-yl)-1-methyl-1H-indazol-6-yl)-3-hydroxypiperidin-1-yl)methyl)cyclohexyl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆): δ: 10.66 (s, 1H), 10.56 (s, 1H), 9.40 (s, 1H), 8.88 (s, 1H), 8.48 (q, 1H), 7.95 (s, 1H), 7.61 (d, J=8.5 Hz, 1H), 7.48 (m, 3H), 7.12 (d, J=8.4 Hz, 1H), 6.37 (t, J=7.2 Hz, 1H), 5.65 (m, 1H), 5.16 (m, 2H), 4.98 (m, 1H), 4.22-3.95 (m, 5H), 3.07 (m, 8H), 2.76 (m, 3H), 2.59 (m, 1H), 2.13 (m, 2H), 1.89 (m, 4H), 1.56 (m, 9H), 1.24 (m, 2H). LCMS (ES⁺): m/z 808.35 [M+H]⁺.

### Example 172 was Prepared Substantially Following the Synthesis of Example 47

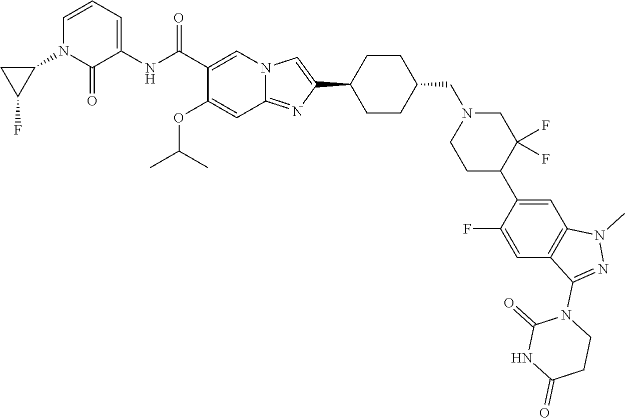 [SMILES: CC(C)Oc1cc2nc([C@H]3CC[C@H](CN4CCC(c5cc6c(cc5F)c(N5CCC(=O)NC5=O)nn6C)C(F)(F)C4)CC3)cn2cc1C(=O)Nc1cccn([C@H]2C[C@H]2F)c1=O]

### 2-((1r, 4S)-4-((4-(3-(2,4-dioxotetrahydropyrimidin-1 (2H)-yl)-5-fluoro-1-methyl-1H-indazol-6-yl)-3,3-difluoropiperidin-1-yl)methyl)cyclohexyl)-N-(1-((1 S, 2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆): δ: 10.60 (d, J=24.5 Hz, 2H), 9.43 (d, J=6.5 Hz, 1H), 8.48 (d, J=7.4 Hz, 1H), 8.06 (d, J=34.4 Hz, 1H), 7.73 (s, 1H), 7.44 (m, 3H), 6.37 (t, J=7.2 Hz, 1H), 5.18 (m, 1H), 4.97 (m, 1H), 4.03 (m, 3H), 3.92 (m, 2H), 3.11 (m, 6H), 2.77 (m, 3H), 2.45 (m, 2H), 2.00 (m, 2H), 1.57 (m, 15H), 1.19 (m, 2H). LCMS (ES⁺): m/z 846.31 [M+H]⁺

### Example 173 was Prepared Substantially Following the Synthesis of Example 47

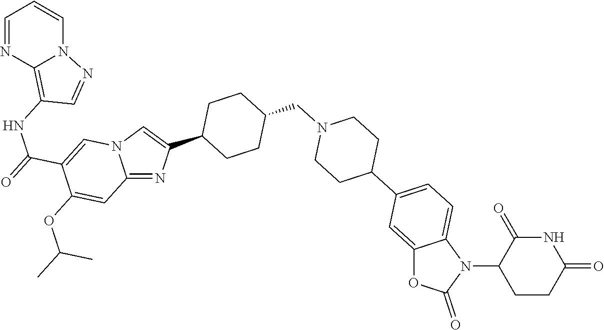 [SMILES: CC(C)Oc1cc2nc([C@H]3CC[C@H](CN4CCC(c5ccc6c(c5)oc(=O)n6C5CCC(=O)NC5=O)CC4)CC3)cn2cc1C(=O)Nc1cnn2cccnc12]

### 2-((1r,4r)-4-((4-(3-(2,6-dioxopiperidin-3-yl)-2-oxo-2,3-dihydrobenzo[d]oxazol-6-yl)piperidin-1-yl)methyl)cyclohexyl)-7-isopropoxy-N-(pyrazolo[1,5-a]pyrimidin-3-yl)imidazo[1,2-a]pyridine-6-carboxamide

¹H NMR (400 MHz, DMSO-d₆): δ: 11.20 (s, 1H), 10.52 (d, J=2.7 Hz, 1H), 9.18 (d, J=4.2 Hz, 1H), 9.09 (q, 1H), 8.77 (d, J=2.0 Hz, 1H), 8.54 (q, 1H), 8.40 (s, 3H), 7.73 (d, J=30.6 Hz, 1H), 7.32 (s, 1H), 7.21-7.12 (m, 4H), 5.34 (m, 1H), 5.05 (m, 1H), 2.92 (m, 4H), 2.63 (m, 4H), 2.21 (m, 4H), 2.00 (m, 4H), 1.81 (m, 9H), 1.54 (m, 2H), 1.41 (m, 1H). LCMS (ES⁺): m/z 760.25 [M+H]⁺.

### Example 174 was Prepared Substantially Following the Synthesis of Example 47

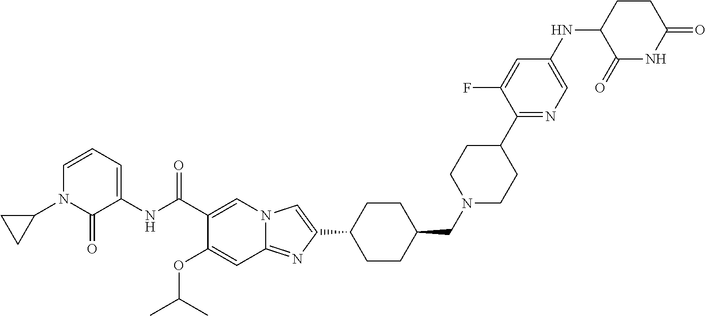 [SMILES: CC(C)Oc1cc2nc([C@H]3CC[C@H](CN4CCC(c5ncc(NC6CCC(=O)NC6=O)cc5F)CC4)CC3)cn2cc1C(=O)Nc1cccn(C2CC2)c1=O]

### N-(1-cyclopropyl-2-oxo-1,2-dihydropyridin-3-yl)-2-((1r,4r)-4-((4-(5-((2,6-dioxopiperidin-3-yl)amino)-3-fluoropyridin-2-yl)piperidin-1-yl)methyl)cyclohexyl)-7-isopropoxyimidazo[1,2-a]pyridine-6-carboxamide

¹H NMR (400 MHz, DMSO-d₆): δ 10.84 (s, 1H), 10.66 (s, 1H), 9.43 (s, 1H), 8.92 (s, 1H), 8.44 (q, 1H), 7.94 (d, J=43.7 Hz, 2H), 7.38-6.93 (m, 4H), 6.43 (d, J=6.6 Hz, 1H), 6.32 (t, J=7.2 Hz, 1H), 5.20 (m, 1H), 4.40 (m, 1H), 3.56-3.09 (m, 8H), 2.69 (m, 2H), 2.11 (m, 1H), 1.93 (m, 8H), 1.57 (m, 9H), 1.21 (m, 2H), 1.06 (m, 2H), 0.91 (m, 2H). LCMS (ES⁺): m/z 753.28 [M+H]⁺.

### Example 175 was Prepared Substantially Following the Synthesis of Example 47

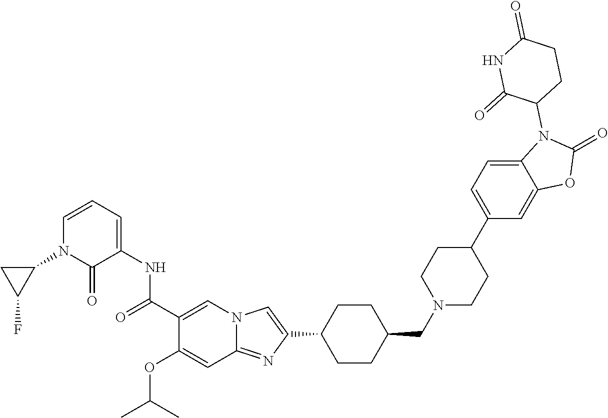 [SMILES: CC(C)Oc1cc2nc([C@H]3CC[C@H](CN4CCC(c5ccc6c(c5)oc(=O)n6C5CCC(=O)NC5=O)CC4)CC3)cn2cc1C(=O)Nc1cccn([C@H]2C[C@H]2F)c1=O]

### 2-((1r,4S)-4-((4-(3-(2,6-dioxopiperidin-3-yl)-2-oxo-2,3-dihydrobenzo[d]oxazol-6-yl)piperidin-1-yl)methyl)cyclohexyl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆): δ: 11.20 (s, 1H), 10.75 (s, 1H), 9.17 (s, 1H), 8.48 (d, J=7.3 Hz, 1H), 8.14 (s, 1H), 7.73 (d, J=30.5 Hz, 1H), 7.43 (d, J=6.8 Hz, 1H), 7.32 (s, 1H), 7.15 (m, 3H), 6.34 (t, J=7.2 Hz, 1H), 5.35 (m, 1H), 5.05 (m, 2H), 3.32 (m, 6H), 2.89 (m, 5H), 2.51-1.75 (m, 20H), 1.48 (m, 1H). LCMS (ES⁺): m/z 794.19 [M+H]⁺.

### Example 176 was Prepared Substantially Following the Synthesis of Example 47

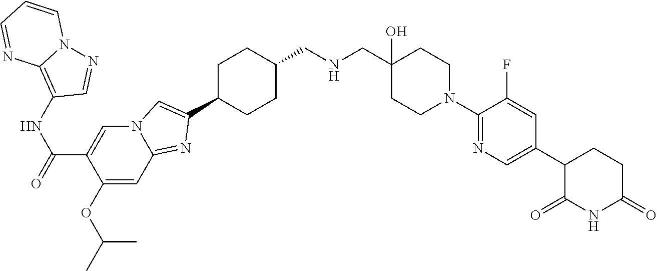 [SMILES: CC(C)Oc1cc2nc([C@H]3CC[C@H](CNCC4(O)CCN(c5ncc(C6CCC(=O)NC6=O)cc5F)CC4)CC3)cn2cc1C(=O)Nc1cnn2cccnc12]

### 2-[4-[[[ ]-[5-(2,6-dioxo-3-piperidyl)-3-fluoro-2-pyridyl]-4-hydroxy-4-piperidyl]methylamino]methyl]cyclohexyl]-7-isopropoxy-N-pyrazolo[1,5-a]pyrimidin-3-yl-imidazo[1,2-a]pyridine-6-carboxamide

¹H NMR (400 MHz, DMSO-d₆): δ: 10.87 (s, 1H), 10.50 (s, 1H), 9.32 (s, 1H), 9.12 (q, 1H), 8.75 (s, 1H), 8.57 (q, 1H), 8.23 (s, 2H), 7.92 (d, J=26.4 Hz, 2H), 7.46 (q, 1H), 7.37 (s, 1H), 7.09 (q, 1H), 5.25 (s, 1H), 5.13 (m, 1H), 3.86 (m, 1H), 3.68 (m, 2H), 3.32 (m, 2H), 3.02 (m, 2H), 2.78 (m, 4H), 2.56 (m, 1H), 2.25 (m, 1H), 2.12 (m, 2H), 1.97 (m, 4H), 1.71-1.54 (m, 11H), 1.20 (m, 2H). LCMS (ES⁺): m/z 767.17 [M+H]⁺.

### Example 177 was Prepared Substantially Following the Synthesis of Example 47

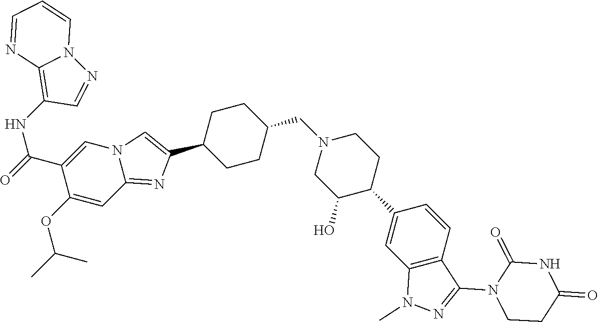 [SMILES: CC(C)Oc1cc2nc([C@H]3CC[C@H](CN4CC[C@H](c5ccc6c(N7CCC(=O)NC7=O)nn(C)c6c5)[C@H](O)C4)CC3)cn2cc1C(=O)Nc1cnn2cccnc12]

### 2-((1S,4r)-4-(((3S,4R)-4-(3-(2,4-dioxotetrahydropyrimidin-1(2H)-yl)-1-methyl-1H-indazol-6-yl)-3-hydroxypiperidin-1-yl)methyl)cyclohexyl)-7-isopropoxy-N-(pyrazolo[1,5-a]pyrimidin-3-yl)imidazo[1,2-a]pyridine-6-carboxamide

¹H NMR (400 MHz, DMSO-d₆): δ: 10.53 (m, 2H), 9.33 (s, 1H), 9.12 (q, 1H), 8.89 (s, 1H), 8.76 (d, J=4.3 Hz, 1H), 8.57 (q, 1H), 7.94 (s, 1H), 7.61 (d, J=8.5 Hz, 1H), 7.39 (d, J=30.0 Hz, 1H), 7.11 (m, 2H), 5.65 (m, 1H), 5.14 (m, 1H), 4.23 (m, 1H), 3.95 (m, 3H), 3.89 (m, 2H), 3.10-2.76 (m, 10H), 2.60 (m, 1H), 2.14 (m, 2H), 1.90 (m, 4H), 1.55 (m, 8H), 1.24 (m, 2H). LCMS (ES⁺): m/z 774.41 [M+H]⁺.

### Example 178 was Prepared Substantially Following the Synthesis of Example 47

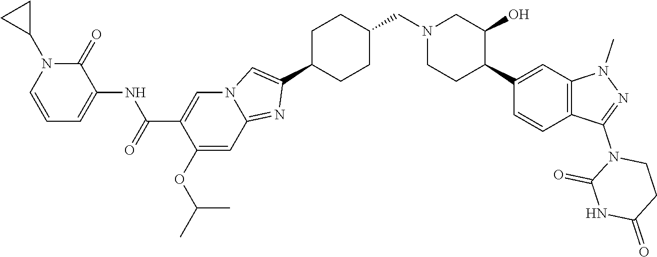 [SMILES: CC(C)Oc1cc2nc([C@H]3CC[C@H](CN4CC[C@H](c5ccc6c(N7CCC(=O)NC7=O)nn(C)c6c5)[C@H](O)C4)CC3)cn2cc1C(=O)Nc1cccn(C2CC2)c1=O]

### N-(1-cyclopropyl-2-oxo-1,2-dihydropyridin-3-yl)-2-((S,4r)-4-(((3S,4R)-4-(3-(2,4-dioxotetrahydropyrimidin-1(2H)-yl)-1-methyl-1H-indazol-6-yl)-3-hydroxypiperidin-1-yl)methyl)cyclohexyl)-7-isopropoxyimidazo[1,2-a]pyridine-6-carboxamide

¹H NMR (400 MHz, DMSO-d₆): δ: 10.76 (s, 1H), 10.53 (s, 1H), 9.16 (s, 1H), 8.42 (m, 1H), 7.72 (d, J=28.8 Hz, 1H), 7.52 (d, J=8.5 Hz, 1H), 7.45 (s, 1H), 7.31 (q, 1H), 7.13 (m, 2H), 6.29 (t, J=7.2 Hz, 1H), 5.03 (m, 1H), 3.93 (m, 6H), 3.82 (m, 1H), 3.50 (m, 1H), 2.97 (m, 2H), 2.75 (m, 3H), 2.58 (m, 1H), 2.37 (m, 2H), 2.19 (m, 3H), 2.07 (m, 2H), 1.97 (m, 3H), 1.77 (m, 2H), 1.52 (m, 6H), 1.43 (m, 2H), 1.04 (m, 3H), 0.90 (m, 2H). LCMS (ES⁺): m/z 790.30 [M+H]⁺.

### Example 179 was Prepared Substantially Following the Synthesis of Example 47

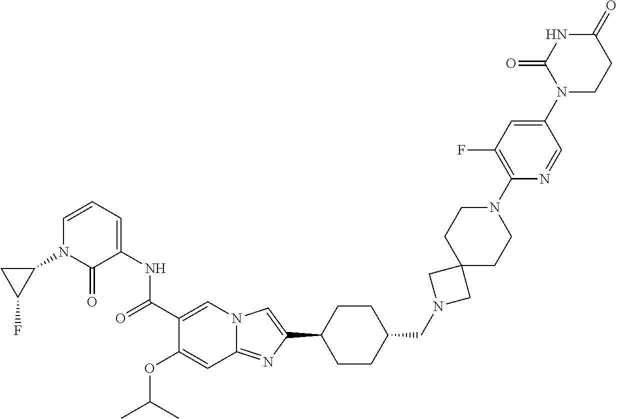 [SMILES: CC(C)Oc1cc2nc([C@H]3CC[C@H](CN4CC5(CCN(c6ncc(N7CCC(=O)NC7=O)cc6F)CC5)C4)CC3)cn2cc1C(=O)Nc1cccn([C@H]2C[C@H]2F)c1=O]

2-((1r,4S)-4-((7-(5-(2,4-dioxotetrahydropyrimidin-1(2H)-yl)-3-fluoropyridin-2-yl)-2,7-diazaspiro[3.5]nonan-2-yl)methyl)cyclohexyl)-N-(1-((1S,2R)-2-fluorocyclopropyl)-2-oxo-1,2-dihydropyridin-3-yl)-7-isopropoxyimidazo[1,2-a]pyridine-6-carboxamide H NMR (400 MHz, DMSO-d₆): δ 10.63 (s, 1H), 10.46 (s, 1H), 9.71 (s, 1H), 9.45 (d, J=3.7 Hz, 1H), 8.48 (d, J=7.4 Hz, 1H), 8.02 (d, J=2.0 Hz, 2H), 7.63 (q, 1H), 7.50 (d, J=6.9 Hz, 1H), 7.42 (s, 1H), 6.38 (t, J=7.2 Hz, 1H), 5.30-4.95 (m, 2H), 4.08 (q, 2H), 4.00-3.53 (m, 4H), 3.65-3.35 (m, 3H), 3.33-3.18 (m, 3H), 3.16 (t, J=5.8 Hz, 1H), 2.81 (t, J=12.2 Hz, 1H), 2.71-2.66 (m, 2H), 2.22-2.03 (t, J=9.3 Hz, 2H), 2.00-1.82 (m, 5H), 1.75-1.42 (m, 11H), 1.32-1.13 (m, 2H). LCMS (ES⁺): m/z 798.81 [M+H]⁺

### Example 180 was Prepared Substantially Following the Synthesis of Example 47

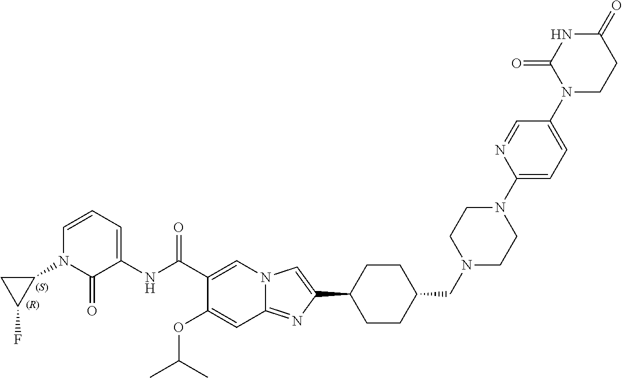

### 2-((1r,4S)-4-((4-(5-(2,6-dioxopiperidin-3-yl)pyridin-2-yl)piperazin-1-yl)methyl)cyclohexyl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆): δ 10.84 (s, 1H), 10.64 (s, 1H), 9.46 (s, 1H), 9.35 (s, 1H), 8.48 (q, 1H), 8.03 (d, J=2.5 Hz, 2H), 7.51 (q, 2H), 7.43 (s, 1H), 6.97 (d, J=8.9 Hz, 1H), 6.38 (t, J=7.2 Hz, 1H), 5.30-4.89 (m, 2H), 4.37 (d, J=13.4 Hz, 2H), 3.80 (q, 1H), 3.76-3.61 (m, 2H), 3.50-3.40 (m, 1H), 3.21 (t, J=11.6 Hz, 2H), 3.09 (s, 4H), 2.83 (t, J=12.3 Hz, 1H), 2.75-2.67 (m, 2H), 2.35-2.15 (m, 3H), 2.08-1.87 (m, 4H), 1.72-1.41 (m, 9H), 1.19 (t, J=11.9 Hz, 2H). LCMS (ES⁺): m/z 739.26 [M+H]⁺

### Example 181 was Prepared Substantially Following the Synthesis of Example 47

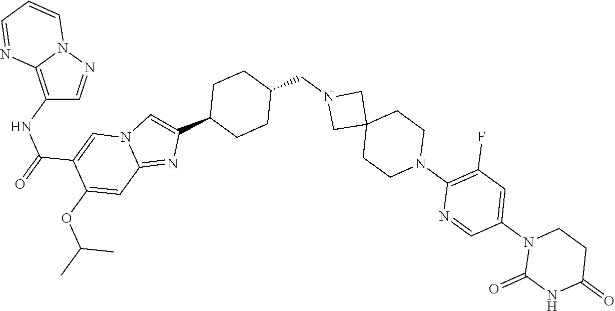 [SMILES: CC(C)Oc1cc2nc([C@H]3CC[C@H](CN4CC5(CCN(c6ncc(N7CCC(=O)NC7=O)cc6F)CC5)C4)CC3)cn2cc1C(=O)Nc1cnn2cccnc12]

### 2-((1r,4r)-4-((7-(5-(2,4-dioxotetrahydropyrimidin-1(2H)-yl)-3-fluoropyridin-2-yl)-2,7-diazaspiro[3.5]nonan-2-yl)methyl)cyclohexyl)-7-isopropoxy-N-(pyrazolo[1,5-a]pyrimidin-3-yl)imidazo[1,2-a]pyridine-6-carboxamide

¹H NMR (400 MHz, DMSO-d₆): δ 10.48 (d, J=13.3 Hz, 2H), 9.68 (s, 1H), 9.36 (d, J=5.5 Hz, 1H), 9.12 (d, J=6.9 Hz, 1H), 8.75 (s, 1H), 8.57 (d, J=3.7 Hz, 1H), 8.02 (t, J=8.5 Hz, 2H), 7.63 (d, J=14.2 Hz, 1H), 7.40 (s, 1H), 7.10 (q, 1H), 5.15 (t, J=5.8 Hz, 1H), 4.09 (t, J=6.4 Hz, 2H), 3.89 (q, 3H), 3.41 (s, 2H), 3.30 (s, 2H), 3.22-3.17 (m, 3H), 2.81 (d, J=11.1 Hz, 1H), 2.72 (d, J=6.6 Hz, 2H), 2.12 (d, J=11.1 Hz, 1H), 1.92 (d, J=26.8 Hz, 6H), 1.7-1.62 (m, 1H), 1.54-1.42 (m, 8H), 1.18 (t, J=7.0 Hz, 2H). LCMS (ES⁺): m/z 764.25 [M+H]⁺

### Example 182 was Prepared Substantially Following the Synthesis of Example 47

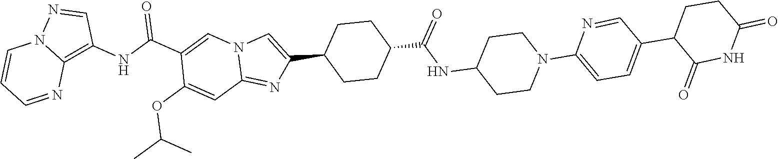 [SMILES: CC(C)Oc1cc2nc([C@H]3CC[C@H](C(=O)NC4CCN(c5ccc(C6CCC(=O)NC6=O)cn5)CC4)CC3)cn2cc1C(=O)Nc1cnn2cccnc12]

### 2-((1r,4r)-4-((1-(5-(2,6-dioxopiperidin-3-yl)pyridin-2-yl)piperidin-4-yl)carbamoyl)cyclohexyl)-7-isopropoxy-N-(pyrazolo[1,5-a]pyrimidin-3-yl)imidazo[,2-a]pyridine-6-carboxamide

¹H NMR (400 MHz, DMSO-d₆) δ: 11.12 (s, 1H), 9.50 (d, J=6.3 Hz, 1H), 8.78 (d, J=7.7 Hz, 1H), 8.38 (d, J=4.3 Hz, 1H), 8.25 (d, J=5.7 Hz, 1H), 6.81 (m, 1H), 5.40 (q, 1H), 5.18 (d, J=82.1 Hz, 1H), 4.77 (d, J=18.5 Hz, 1H), 4.16 (d, J=11.5 Hz, 1H), 3.78 (q, 1H), 2.75 (m, 1H), 1.99 (m, 1H), 1.72 (s, 1H), 1.46 (m, 1H), 0.98 (s, 1H). LCMS (ES⁺): m/z 733.27 [M+H]⁺

### Example 183 was Prepared Substantially Following the Synthesis of Example 47

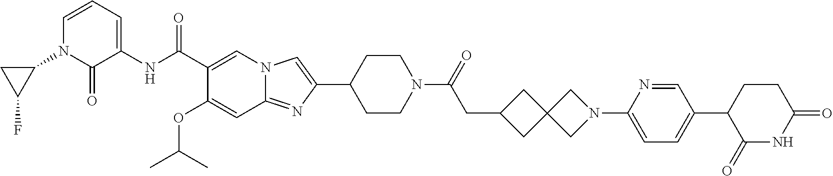 [SMILES: CC(C)Oc1cc2nc(C3CCN(C(=O)CC4CC5(C4)CN(c4ccc(C6CCC(=O)NC6=O)cn4)C5)CC3)cn2cc1C(=O)Nc1cccn([C@H]2C[C@H]2F)c1=O]

### 2-(1-(2-(2-(5-(2,6-dioxopiperidin-3-yl)pyridin-2-yl)-2-azaspiro[3.3]heptan-6-yl)acetyl)piperidin-4-yl)-N-(1-((1S,2R)-2-fluorocyclopropyl)-2-oxo-1,2-dihydropyridin-3-yl)-7-isopropoxyimidazo[1,2-a]pyridine-6-carboxamide

¹H NMR (400 MHz, DMSO-d₆) δ: 10.79 (s, 1H), 10.74 (s, 1H), 9.17 (s, 1H), 8.48-8.46 (dd, J=1.6, 7.6 Hz, 1H), 7.88 (d, J=2 Hz, 1H), 7.74 (s, 1H), 7.43 (d, J=7.2 Hz, 1H), 7.35 (dd, J=2.4, 8.4 Hz, 1H), 7.16 (s, 1H), 6.35-6.31 (m, 2H), 5.04 (m, 2H), 4.44 (d, 1H), 3.94 (m, 3H), 3.81 (s, 2H), 3.71 (dd, J=4.8, 12.4 Hz, 1H), 3.46-3.45 (m, 1H), 3.15 (t, 1H), 2.92 (m, 1H), 2.71-2.66 (m, 2H), 2.49 (s, 3H), 2.33-2.30 (m, 2H), 2.22-1.87 (m, 6H), 1.59-1.42 (m, 11H). LCMS (ES⁺): m/z 779.19 [M+H]⁺

### Example 184 was Prepared Substantially Following the Synthesis of Example 113

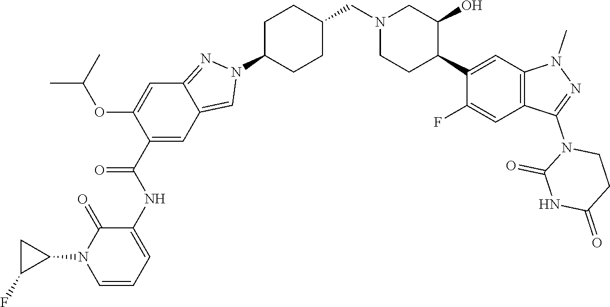 [SMILES: CC(C)Oc1cc2nn([C@H]3CC[C@H](CN4CC[C@H](c5cc6c(cc5F)c(N5CCC(=O)NC5=O)nn6C)[C@H](O)C4)CC3)cc2cc1C(=O)Nc1cccn([C@H]2C[C@H]2F)c1=O]

### 2-((1S,4S)-4-(((3S,4R)-4-(3-(2,4-dioxotetrahydropyrimidin-1(2H)-yl)-5-fluoro-1-methyl-1H-indazol-6-yl)-3-hydroxypiperidin-1-yl)methyl)cyclohexyl)-N-(1-((1S,2R)-2-fluorocyclopropyl)-2-oxo-1,2-dihydropyridin-3-yl)-6-isopropoxy-2H-indazole-5-carboxamide

¹H-NMR (400 MHz, DMSO-d₆): δ 10.86 (s, 1H), 10.54 (s, 1H), 8.51-8.57 (m, 3H), 7.56 (d, J=6.0 Hz, 1H), 7.34-7.41 (m, 2H), 7.25 (s, 1H), 6.30 (t, J=7.0 Hz, 1H), 5.20-4.99 (m, 2H), 4.45 (br s, 1H), 4.09 (d, J=7.6 Hz, 1H), 3.98 (s, 3H), 3.89 (t, J=6.8 Hz, 2H), 3.81-3.82 (m, 1H), 2.98-3.05 (m, 4H), 2.74 (t, J=7.0 Hz, 2H), 1.91-2.23 (m, 10H), 1.72-1.65 (m, 11H), 1.12-1.15 (m, 2H). LCMS (ES⁺): m/z 826.59 [M+H]⁺

### Example 185 was Prepared Substantially Following the Synthesis of Example 15

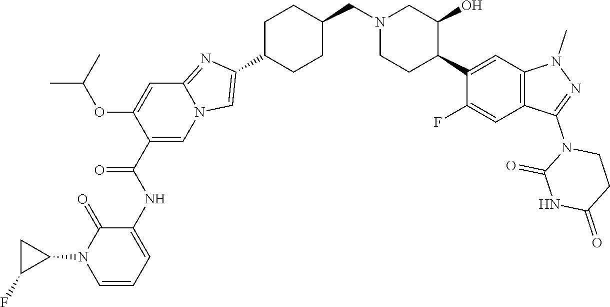

### 2-((1S,4S)-4-(((3S,4R)-4-(3-(2,4-dioxotetrahydropyrimidin-1(2H)-yl)-5-fluoro-1-methyl-1H-indazol-6-yl)-3-hydroxypiperidin-1-yl)methyl)cyclohexyl)-N-(1-((1S,2R)-2-fluorocyclopropyl)-2-oxo-1,2-dihydropyridin-3-yl)-7-isopropoxyimidazo[1,2-a]pyridine-6-carboxamide

¹H-NMR (400 MHz, DMSO-d₆): 10.66 (s, 1H), 10.57 (s, 1H), 9.41 (s, 1H), 8.47 (q, 1H), 8.76 (s, 1H), 7.97 (s, 1H), 7.49-7.38 (m, 4H), 6.37 (t, 1H), 5.74 (s, 1H), 5.29-4.95 (m, 3H), 4.17 (s, 1H), 4.00 (s, 3H), 3.93 (m, 2H), 3.45-3.20 (m, 5H), 3.05 (m, 2H), 2.90-2.62 (m, 4H), 2.15 (m, 2H), 1.90 (m, 4H), 1.70-1.40 (m, 11H), 1.23 (m, 2H). LCMS (ES⁺): m/z 826.52 [M+H]⁺

### Synthesis of Example 186 2-(1-(2-(1-(5-(2,6-dioxopiperidin-3-yl)-3-fluoropyridin-2-yl)piperidin-4-yl)acetyl)piperidin-4-yl)-N-(1-((1S,2R)-2-fluorocyclopropyl)-2-oxo-1,2-dihydropyridin-3-yl)-7-isopropoxyimidazo[1,2-a]pyrimidine-6-carboxamide

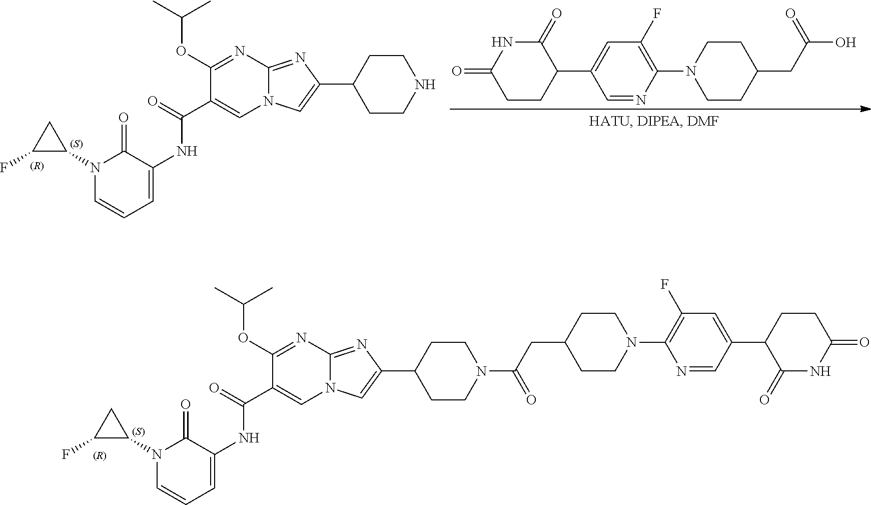

To a stirred solution of 7-isopropoxy-N-[2-oxo-1-[(1S,2R)-2-fluorocyclopropyl]-3-pyridyl]-2-(4-piperidyl)imidazo[1,2-a]pyrimidine-6-carboxamide (245.38 mg, 431.61 μmol, TFA salt) and 2-[1-[5-(2,6-dioxo-3-piperidyl)-3-fluoro-2-pyridyl]-4-piperidyl]acetic acid (0.200 g, 431.61 μmol, TFA salt) in DMF (2 mL) were added DIPEA (167.35 mg, 1.29 mmol, 225.54 μL) and HATU (180.52 mg, 474.77 μmol) at 0° C. The reaction was warmed to room temperature and stirred at 25° C. for 3 h. After complete consumption of the starting material, the reaction mixture was concentrated under reduced pressure to give the crude product, which was purified by prep-HPLC to afford 2-[1-[2-[1-[5-(2,6-dioxo-3-piperidyl)-3-fluoro-2-pyridyl]-4-piperidyl] acetyl]-4-piperidyl]-7-isopropoxy-N-[2-oxo-1-[(1S,2R)-2-fluorocyclopropyl]-3-pyridyl] imidazo[1,2-a] pyrimidine-6-carboxamide (0.1 g, 127.10 μmol, 29.45% yield) as white solid.

### Preparative-HPLC Conditions:

Column/dimensions: X-BRIDGE C18 (19*250*5μ)

Mobile phase A: 10 MM AA in Water

Mobile phase B: 100% Acetonitrile

Gradient (Time % B): 0/10, 2/20, 13.50/58, 13.60/98, 16/98, 16.10/10, 19/10

Flow rate: 17 mL/min

Solubility: Water+Acetonitrile

¹H NMR (400 MHz, DMSO-d₆): δ 10.98 (s, 1H), 10.68 (s, 1H), 9.48 (s, 1H), 8.45 (m, 1H), 7.86 (s, 1H), 7.63 (s, 1H), 7.40 (m, 2H), 6.34 (t, J=7.2 Hz, 1H), 5.60 (m, 1H), 5.00 (m, 1H), 4.44 (m, 1H), 3.91 (m, 3H), 3.81 (m, 1H), 3.47 (m, 1H), 2.91 (m, 1H), 2.50 (m, 7H), 2.22 (m, 2H), 1.96 (m, 4H), 1.74 (m, 2H), 1.47 (m, 10H), 1.31 (m, 2H). LCMS (ES⁺): m/z 786.53 [M+H]⁺

The following examples were made similar to the examples listed above:

| | | LCMS | |
|---|---|---|---|
| | | (ES+): | |
| Example | | m/z | |
| Number | Structure | [M + H]⁺ | NMR |
| 187 | 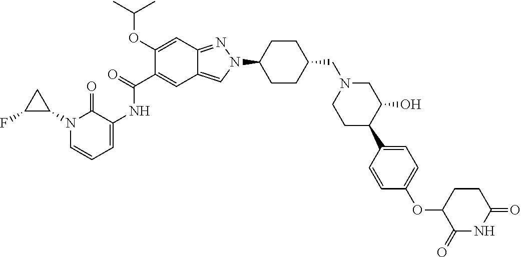 [SMILES: CC(C)Oc1cc2nn([C@H]3CC[C@H](CN4CC[C@H](c5ccc(OC6CCC(=O)NC6=O)cc5)[C@@H](O)C4)CC3)cc2cc1C(=O)Nc1cccn([C@H]2C[C@H]2F)c1=O] | 769.63 | ¹H NMR (400 MHz, DMSO) δ 10.88 (s, 1H), 10.86 (s, 1H), 8.57 (s, 1H), 8.55 (s, 1H), 8.52-8.50 (d, J = 7.2 Hz, 1H), 8.20 (s, 1H), 7.40 (d, J = 7.2 Hz, 1H), 7.24 (s, 1H), 7.15 (d, J = 8.4 Hz, 1H), 6.91 (d, J = 8.8 Hz, 1H), 6.31 (t, J = 7.6 Hz, 1H), 5.14 (bs, 1H), 5.04-4.94 (m, 2H), 3.58-3.40 (bs, 2H), 3.06 (bs, 1H), 2.84 (bs, 1H), 2.76-2.53 (m, 4H), 2.33- 2.04 (bs, 7H), 1.99-1.82 (m, 5H), 1.68-1.56 (bs, |
| | | | 4H), 1.52 (d, J = 11.6 Hz, |
| | | | 6H). |
| 188 | 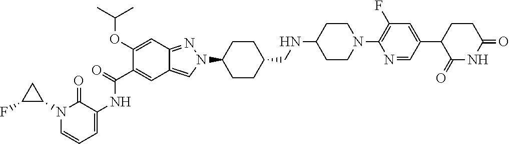 [SMILES: CC(C)Oc1cc2nn([C@H]3CC[C@H](CNC4CCN(c5ncc(C6CCC(=O)NC6=O)cc5F)CC4)CC3)cc2cc1C(=O)Nc1cccn([C@H]2C[C@H]2F)c1=O] | 753.54 | ¹H NMR (400 MHz, DMSO-D6) δ 10.85 (s, 2H), 8.55 (d, J = 6.0 Hz, 2H), 8.48 (dd, J = 7.4, 1.4 Hz, 1H), 8.37 (s, 1H), 7.87 (s, 1H), 7.43 (dd, J = 14.4, 1.6 Hz, 1H), 7.28 (dd, J = 7.2, 1.6 Hz, 1H), 7.24 (s, 1H), 6.25 (t, J = 7.2 Hz, |
| | | | 1H), 4.99-4.97 (m, 1H), |
| | | | 4.45-4.38 (m, 1H), 3.90- |
| | | | 3.82 (m, 3H), 3.51-3.48 |
| | | | (m, 1H), 2.95-2.89 (m, |
| | | | 3H), 2.69-2.63 (m, 5H), |
| | | | 2.17-2.14 (m, 1H), 1.99 |
| | | | (d, J = 4.0 Hz, 2H), 1.97 |
| | | | 1.88 (m, 7H), 1.52 (d, J = |
| | | | 6.0 Hz, 6H), 1.48 (s, 1H), |
| | | | 1.17-1.14 (m, 2H), 1.05- |
| | | | 1.03 (m, 2H), 1.02-0.98 |
| | | | (m, 2H), 0.92-0.90 (m, |
| | | | 2H). |
| 189 | 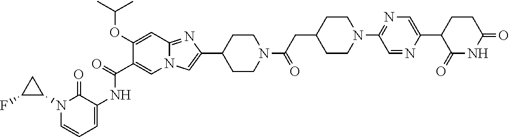 [SMILES: CC(C)Oc1cc2nc(C3CCN(C(=O)CC4CCN(c5cnc(C6CCC(=O)NC6=O)cn5)CC4)CC3)cn2cc1C(=O)Nc1cccn([C@H]2C[C@H]2F)c1=O] | 768.24 | ¹H NMR (400 MHz, DMSO-d6) δ: 10.74 (s, 1H), 10.80 (s, 1H), 9.17 (s, 1H), 8.52 (s, 1H), 8.47 (dd, J = 7.2 Hz, 1.6 Hz, 1H), 8.23 (d, J = 1.2 Hz, 1H), 8.10 (d, J = 1.2 Hz, 1H), 7.73 (s, 1H), 7.43 (m, 1H), 7.15 (s, 1H), 6.33 (t, |
| | | | J = 7.2 Hz, 1H), 5.01 (m, |
| | | | 2H), 4.41 (m, 1H), 4.31 |
| | | | (m, 2H), 3.98 (m, 1H), |
| | | | 3.89 (m, 1H), 3.45 (m, |
| | | | 1H), 3.16 (m, 1H), 2.90 |
| | | | (m, 3H), 2.87 (m, 1H), |
| | | | 2.32 (m, 2H), 2.07 (m, |
| | | | 1H), 2.02 (m, 4H), 1.75 |
| | | | (d, J = 11.2 Hz, 2H), 1.47 |
| | | | (m, 10 H), 1.17 (m, 3H) |
| 190 | 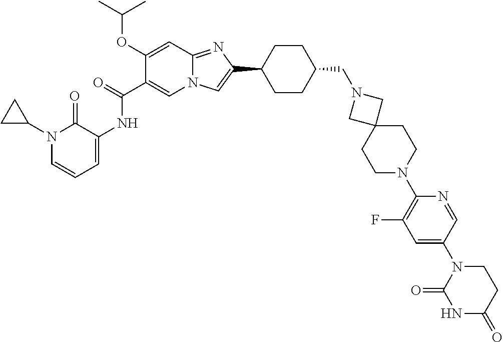 [SMILES: CC(C)Oc1cc2nc([C@H]3CC[C@H](CN4CC5(CCN(c6ncc(N7CCC(=O)NC7=O)cc6F)CC5)C4)CC3)cn2cc1C(=O)Nc1cccn(C2CC2)c1=O] | 780.66 | ¹H NMR (400 MHz, DMSO): δ 10.65 (s, 1H), 10.46 (s, 1H), 9.71 (s, 1H), 9.43 (s, 1H), 8.43 (q, J = 2.9 Hz, 1H), 8.02 (s, 2H), 7.63 (q, J = 5.4 Hz, 1H), 7.70-7.35 (m, 2H), 6.32 (t, J = 7.2 Hz, 1H), 5.31-5.20 (m, 1H), 4.10 (t, J = 8.0 Hz, 2H), 3.91 (q, J = 5.5 Hz, 2H), 3.75 (t, J = 6.7 Hz, 2H), 3.60- 3.40 (m, 1H), 3.40 (s, 2H), 3.31 (s, 2H), 3.16 (d, J = 6.1 Hz, 2H), 2.75 (q, J = 14.8 Hz, 1H), 2.75- 2.60 (m, 2H), 2.11 (d, J = 11.4 Hz, 2H), 2.00-1.82 (m, 5H), 1.75-1.35 (m, 9H), 1.25-1.08 (m, 2H), 1.05 (q, J = 6.8 Hz, 2H), |
| | | | 0.91 (q, J = 5.5 Hz, 2H). |
| 191 | 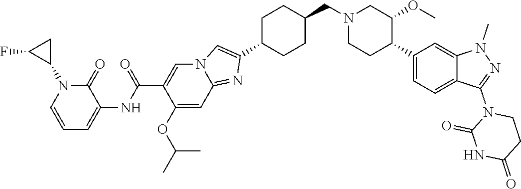 [SMILES: CO[C@H]1CN(C[C@H]2CC[C@H](c3cn4cc(C(=O)Nc5cccn([C@H]6C[C@H]6F)c5=O)c(OC(C)C)cc4n3)CC2)CC[C@H]1c1ccc2c(N3CCC(=O)NC3=O)nn(C)c2c1] | 822.22 | ¹H NMR (400 MHz, DMSO-d6) δ 10.66 (s, 1H), 10.56 (s, 1H), 9.40 (s, 1H), 8.95 (s, 1H), 8.48 (q, 1H), 7.95 (s, 1H), 7.62 (d, J = 8.5 Hz, 1H), 7.48 (d, J = 6.7 Hz, 1H), 7.43 (s, 1H), 7.36 (s, 1H), 7.15 (d, J = 8.6 Hz, 1H), 6.37 (t, J = 7.2 Hz, 1H), 5.15 (m, 1H), 4.97 (m, 1H), 3.99 (s, 1H), 3.93 (t, J = |
| | | | 6.7 Hz, 1H), 3.86 (d, J = |
| | | | 11.3 Hz, 1H), 3.48 (q, |
| | | | 1H), 3.16 (s, 1H), 3.01 (d, |
| | | | J = 7.5 Hz, 1H), 2.76 (t, |
| | | | J = 6.7 Hz, 1H), 2.14 (d, |
| | | | J = 12.6 Hz, 1H), 1.95 (m, |
| | | | 1H), 1.67 (s, 1H), 1.56 (q, |
| | | | 1H), 1.46 (m, 1H), 1.25 |
| | | | (d, J = 9.9 Hz, 1H). |
| 192 | 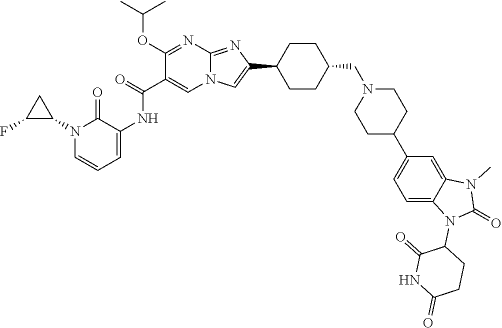 [SMILES: CC(C)Oc1nc2nc([C@H]3CC[C@H](CN4CCC(c5ccc6c(c5)n(C)c(=O)n6C5CCC(=O)NC5=O)CC4)CC3)cn2cc1C(=O)Nc1cccn([C@H]2C[C@H]2F)c1=O] | 808.67 | ¹H NMR (400 MHz, DMSO) δ 11.10 (s, 1H), 10.64 (d, J = 5.4 Hz, 1H), 9.60 (s, 1H), 8.83 (s, 1H), 8.47 (d, J = 7.4 Hz, 1H), 7.76 (d, J = 17.0 Hz, 1H), 7.49 (d, J = 6.5 Hz, 1H), 7.08 (t, J = 8.2 Hz, 2H), 6.92 (d, J = 8.0 Hz, 1H), 6.37 (t, J = 7.2 Hz, 1H), 5.63 (m, 1H), 5.36 (m, 1H), 5.06 (m, 1H), 3.51 (m, 2H), 3.35 (m, 4H), 3.03-2.9 (m, 6H), 2.70- 2.50 (m, 2H), 2.03-1.8 (m, 10H), 1.60-1.54 (m, 10H), 1.21 (m, 2H). |
| 193 | 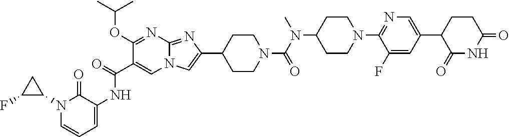 [SMILES: CC(C)Oc1nc2nc(C3CCN(C(=O)N(C)C4CCN(c5ncc(C6CCC(=O)NC6=O)cc5F)CC4)CC3)cn2cc1C(=O)Nc1cccn([C@H]2C[C@H]2F)c1=O] | 801.17 | ¹H NMR (400 MHz, DMSO) δ: 10.88 (s, 1H), 10.69 (s, 1H), 9.46 (s, 1H), 8.47 (dd, J = 7.2, 1.6 Hz, 1H), 7.88 (s, 1H), 7.63 (s, 1H), 7.47-7.43 (m, 2H), 6.35 (t, J = 7.2 Hz, 1H), 5.62-5.61 (m, 1H), 5.15- 4.95 (m, 1H), 4.03 (d, J = |
| | | | 12.0 Hz, 2H), 3.86-3.83 |
| | | | (m, 1H), 3.78-3.68 (m, |
| | | | 1H), 3.60 (d, J = 12.4 Hz, |
| | | | 2H), 3.48 (t, J = 2.8 Hz, |
| | | | 1H), 2.91-2.82 (m, 5H), |
| | | | 2.70-2.67 (m, 4H), 2.55- |
| | | | 2.54 (m, 1H), 2.81-2.20 |
| | | | (m, 1H), 2.00-1.98 (m, |
| | | | 3H), 1.95-1.82 (m, 2H), |
| | | | 1.79-1.60 (m, 5H), 1.58- |
| | | | 1.55 (m, 6H), 1.48-1.41 |
| | | | (m, 1H). |
| 194 | 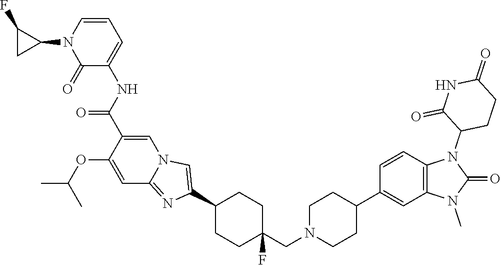 [SMILES: CC(C)Oc1cc2nc([C@H]3CC[C@](F)(CN4CCC(c5ccc6c(c5)n(C)c(=O)n6C5CCC(=O)NC5=O)CC4)CC3)cn2cc1C(=O)Nc1cccn([C@H]2C[C@H]2F)c1=O] | 825.66 | ¹H NMR (400 MHz, DMSO) δ 11.08 (s, 1H), 10.75 (s, 1H), 9.17 (s, 1H), 8.48 (q, 1H), 8.31 (s, 1H), 7.73 (s, 1H), 7.43 (d, J = 5.9 Hz, 1H), 7.14 (d, J = 16.7 Hz, 1H), 7.00 (d, J = 8.1 Hz, 1H), 6.92 (d, J = 7.5 Hz, 1H), 6.34 (t, J = 7.2 Hz, 1H), 5.33 (q, 1H), 5.05 (m, 1H), 3.46 (m, 1H), 3.34 (s, 1H), 3.03 (d, J = 10.9 Hz, 1H), 2.90 (m, 1H), 2.67 (m, 1H), 2.22 (t, J = 9.8 Hz, 1H), 2.00 (m, 1H), 1.72 (q, 1H), 1.52 (q, 1H). |
| 195 | 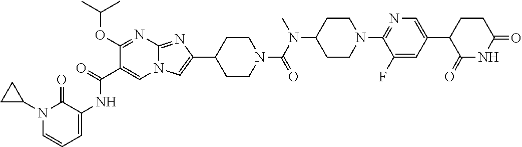 [SMILES: CC(C)Oc1nc2nc(C3CCN(C(=O)N(C)C4CCN(c5ncc(C6CCC(=O)NC6=O)cc5F)CC4)CC3)cn2cc1C(=O)Nc1cccn(C2CC2)c1=O] | 783.19 | ¹H NMR (400 MHz, DMSO) δ: 10.90 (s, 1H), 10.70 (s, 1H), 9.45 (s, 1H), 8.42 (dd, J = 7.2Hz, 1.6 Hz, 1H), 7.88 (s, 1H), 7.62 (s, 1H), 7.45 (dd, J = 14.4 Hz, 1.6 Hz, 1H), 7.33 (dd, J = 6.8 Hz, 1.6 Hz, 1H), 6.29 (t, J = 7.2 Hz, |
| | | | 1H), 5.62-5.60 (m, 1H), |
| | | | 4.04-4.01 (m, 2H), 3.88- |
| | | | 3.81 (m, 1H), 3.79-3.69 |
| | | | (m, 1H), 3.65-3.59 (m, |
| | | | 2H), 3.55-3.49 (m, 1H), |
| | | | 2.90-2.82 (m, 5H), 2.72- |
| | | | 2.66 (m, 4H), 2.50-2.49 |
| | | | (m, 1H), 2.35-2.22 (m, |
| | | | 1H), 2.00-1.94 (m, 3H), |
| | | | 1.82 1.79 (m, 2H), 1.70- |
| | | | 1.60 (m, 4H), 1.54 (d, J = |
| | | | 6.4 Hz, 6H), 1.05-1.04 (m, |
| | | | 2H), 0.93-0.90 (m, 2H). |
| 196 | 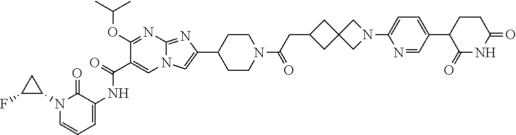 [SMILES: CC(C)Oc1nc2nc(C3CCN(C(=O)CC4CC5(C4)CN(c4ccc(C6CCC(=O)NC6=O)cn4)C5)CC3)cn2cc1C(=O)Nc1cccn([C@H]2C[C@H]2F)c1=O] | 780.62 | ¹H NMR (400 MHz, DMSO) δ: 10.85 (s, 1H), 10.69 (s, 1H), 9.46 (s, 1H), 8.47 (dd, J = 7.2, 1.6 Hz, 1H), 7.88 (d, J = 2.0 Hz, 1H), 7.63 (s, 1H), 7.45 (d, J = 6.0 Hz, 1H), 7.35 (dd, J = 8.4, 2.4 Hz, 1H), 6.34 (dd, J = 14.4, 7.6 Hz, 2H), |
| | | | 5.61 (d, J = 6.4 Hz, 1H), |
| | | | 5.19-4.95 (m, 1H), 4.45- |
| | | | 4.35 (m, 1H), 3.94 (s, 3H), |
| | | | 3.81 (s, 2H), 3.71 (dd, J = |
| | | | 12.0, 4.8 Hz, 1H), 3.48- |
| | | | 3.47 (m, 1H), 3.25-3.16 |
| | | | (m, 1H), 2.99-2.91 (m, |
| | | | 1H), 2.71-2.62 (m, 2H), |
| | | | 2.50 (s, 3H), 2.33-2.32 |
| | | | (m, 2H), 2.25-2.13 (m, |
| | | | 1H), 1.97-1.92 (m, 4H), |
| | | | 1.58-1.42 (m, 12H). |

### Example 197 Synthesis of 2-[4-[[4-[3-(2,4-dioxohexahydropyrimidin-1-yl)-5-fluoro-1-methyl-indazol-6-yl]-1-piperidyl]methyl]cyclohexyl]-N-[1-[(1S,2R)-2-fluorocyclopropyl]-2-oxo-3-pyridyl]-7-isopropoxy-imidazo[1,2-a]pyrimidine-6-carboxamide

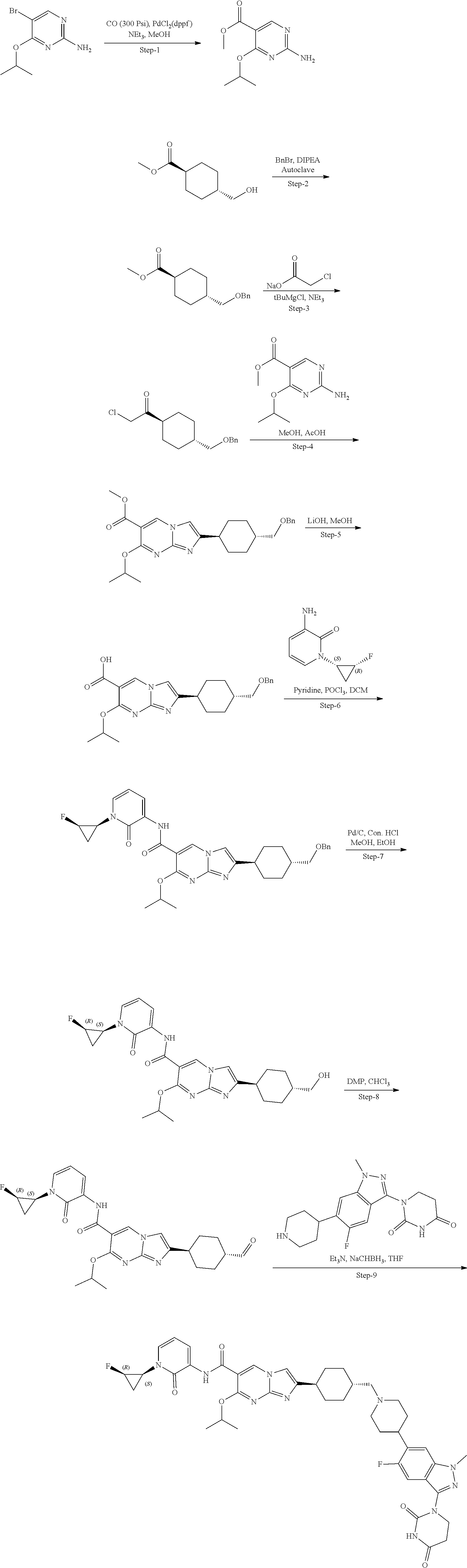

### Step-1:

A parr autoclave (2 L) was charged with 5-bromo-4-isopropoxy-pyrimidin-2-amine (50 g, 215.45 mmol), MeOH (700 mL) and purged with N₂ gas for 10 min. Pd(dppf)Cl₂ (4.72 g, 6.46 mmol), triethylamine (26.16 g, 258.5 mmol, 36 mL) were added and the reaction mixture was filled with CO gas (300 psi) and the resulting mixture was stirred at 100° C. for 16 h. After complete consumption of the starting material, the reaction mixture was filtered through a pad of celite and washed with ethyl acetate (500 mL). The combined filtrate was concentrated under reduced pressure to give the crude product, which was purified by column chromatography using silica gel (100-200 mesh) and 30 to 60% of EtOAc in Pet ether as eluent to afford methyl 2-amino-4-isopropoxypyrimidine-5-carboxylate (31 g, 129.32 mmol, 60% yield) as an off-white solid. LCMS (ES⁺): m/z 212.32 [M+H]⁺

### Step-2:

A 2 L autoclave was charged with methyl 4-(hydroxyethyl) cyclohexane carboxylate (200 g, 1.16 mol), DIPEA (458 mL 2.55 mol), benzyl bromide (337.2 g, 1.97 mol, 234 mL) at room temperature and stirred at 130° C. for 8 h. After complete consumption of the starting material, the reaction mixture was quenched with water (500 mL) and extracted with ethyl acetate (3×500 mL). The combined organic layer was dried over anhydrous sodium sulfate, filtered and concentrated under reduced pressure. The crude product was purified by column chromatography using silica gel (100-200 mesh) and 5-10% of ethyl acetate in Petroleum ether as eluent to afford methyl 4-(benzyloxy-methyl)cyclohexanecarboxylate (230 g, 76% yield) as a yellow liquid. ¹H NMR (400 MHz, DMSO-d₆): δ 7.31 (m, 5H), 4.43 (s, 2H), 3.57 (s, 3H), 3.23 (d, J=8, 2H), 2.23 (m, 1H), 1.83 (d, J=11.6 2H), 1.77 (d, J=11.6 2H), 1.54 (s, 1H), 1.29 (m, 2H), 1.01 (m, 2H).

### Step-3:

To a stirred solution of methyl 4-(benzyloxymethyl)cyclohexanecarboxylate (100 g, 381.18 mmol) in THE (1 L) was added triethylamine (154.29 g, 1.52 mol, 212.52 mL) and sodium chloroacetate (177.60 g, 1.52 mol) at −20° C. and stirred for 1 h. Then tert-butylmagnesium chloride solution (2 M in THF, 762 mL) was added dropwise over a period of 1 h at the same temperature. The reaction mixture was allowed to stir at this temperature for 4 h. After complete consumption of the starting material, the reaction was quenched with saturated cold ammonium chloride solution (400 mL) and extracted with ethyl acetate (2×500 mL). The combined organic layers were washed with brine solution (500 mL), dried over anhydrous Na₂SO₄, filtered and concentrated under reduced pressure to afford 1-[4-(benzyloxymethyl)cyclohexyl]-2-chloro-ethanone (90 g, 85% yield) as a grey solid. ¹H NMR (400 MHz, DMSO-d₆): δ 7.31 (m, 5H), 4.63 (s, 2H), 4.45 (d, J=12, 3H), 3.25 (m, 2H), 2.49 (m, 1H), 1.85 (m, 4H), 1.54 (s, 1H), 1.29 (m, 2H), 1.01 (m, 2H).

### Step-4:

A stirred solution of methyl 2-amino-4-isopropoxy-pyrimidine-5-carboxylate (20 g, 94.69 mmol) and 1-[4-(benzyloxymethyl)cyclohexyl]-2-chloro-ethanone (60.00 g, 213.68 mmol) in methanol (140 mL) was added acetic acid (8.39 g, 139.75 mmol, 8.00 mL) and the mixture was purged with nitrogen gas for 10 min. The reaction mixture was stirred at 100° C. for 48 h. Upon completion of the reaction, the reaction mixture was concentrated under reduced pressure to give the crude compound, which was purified by reverse phase column chromatography (C18 column, 0.1% formic acid in MeCN (0 to 65%). The fractions were combined and extracted with ethyl acetate (2×200 mL), dried over anhydrous sodium sulfate, filtered and concentrated to afford methyl 2-[4-(benzyloxymethyl)cyclohexyl]-7-isopropoxy-imidazo[1,2-a]pyrimidine-6-carboxylate (24 g, 42.70 mmol, 45% yield) as a pale brown semi-solid. LCMS (ES⁺): m/z 438.26 [M+H]⁺

### Step-5:

A stirred solution of methyl 2-[4-(benzyloxymethyl)cyclohexyl]-7-isopropoxy-imidazo[1,2-a]pyrimidine-6-carboxylate (4 g, 9.14 mmol) in methanol (40 mL), THF (40 mL), and water (20 mL) was cooled to 0° C. before lithium hydroxide monohydrate, 98% (1.15 g, 27.43 mmol, 762.13 μL) was added and the reaction mixture was stirred at 50° C. for 1 h. After complete consumption of the starting material, the reaction mixture was concentrated under reduced pressure. The obtained residue was dissolved in water (40 mL) and acidified with 2N HCl until pH=5-6 and extracted with 10% methanol in DCM (3×50 mL). The combined organic layers were dried over anhydrous sodium sulfate, filtered and concentrated under reduced pressure to afford 2-[4-(benzyloxymethyl)cyclohexyl]-7-isopropoxy-imidazo[1,2-a]pyrimidine-6-carboxylic acid (2.5 g, 5.35 mmol, 58.5% yield) as a yellow solid. LCMS (ES⁺): m/z 424.37 [M+H]⁺

### Step-6:

To a stirred solution of 2-[4-(benzyloxymethyl)cyclohexyl]-7-isopropoxy-imidazo[1,2-a]pyrimidine-6-carboxylic acid (2.5 g, 5.90 mmol) 3-amino-1-[(1S,2R)-2-fluorocyclopropyl]pyridin-2-one hydrochloride (1.45 g, 7.08 mmol) in DCM (25 mL) was added pyridine (4.58 g, 57.87 mmol, 4.68 mL) at 0° C. and the reaction mixture was stirred at 0° C. for 5 min. Then phosphoryl trichloride (3.37 g, 21.96 mmol) was added at 0° C. and stirred for 2 hr at rt. After complete consumption of the starting material, water (100 mL) was added to the reaction mixture and extracted with DCM (100 mL). The combined organic phase was washed with brine solution, dried over sodium sulfate, filtered and concentrated under reduced pressure to give 2-[4-(benzyloxymethyl)cyclohexyl]-7-isopropoxy-N-[2-oxo-1-[(1S,2R)-2-fluorocyclopropyl]-3-pyridyl]imidazo[1,2-a]pyridine-6-carboxamide (8.0 g, 13.41 mmol, 62.96% yield) as a brown solid. LCMS (ES⁺): m/z 574.51 [M+H]⁺

### Step-7:

To the stirred solution of 2-[4-(benzyloxymethyl)cyclohexyl]-7-isopropoxy-N-[2-oxo-1-[(1S,2R)-2-fluorocyclopropyl]-3-pyridyl]imidazo[1,2-a]pyrimidine-6-carboxamide (3.75 g, 6.54 mmol) in methanol (37.5 mL) and ethanol (75 mL) was added 10% Palladium on carbon 50% wet basis (3.75 g, 35.23 mmol) and hydrochloric acid, 36% w/w aq. soln. (238.34 mg, 6.54 mmol, 297.92 μL) and the reaction was stirred for 2 h at 25° C. under hydrogen atmosphere. Upon completion of the reaction, the reaction mixture was filtered through celite using 10% MeOH in DCM (100 mL) and the filtrate was concentrated under reduced pressure. The obtained crude product was dissolved in 10% MeOH in DCM (100 mL) and washed with aqueous NaHCO₃ solution. The organic layer was dried over anhydrous sodium sulfate and concentrated under reduced pressure to afford 2-[4-(hydroxymethyl)cyclohexyl]-7-isopropoxy-N-[2-oxo-1-[rac-(1S,2R)-2-fluorocyclopropyl]-3-pyridyl]imidazo[1,2-a]pyrimidine-6-carboxamide (2 g, 3.72 mmol, 56.95% yield) as a yellow solid. LCMS (ES⁺): m/z 484.29 [M+H]⁺

### Step-8:

To a stirred solution of 2-[4-(hydroxymethyl)cyclohexyl]-7-isopropoxy-N-[2-oxo-1-[(1S,2R)-2-fluorocyclopropyl]-3-pyridyl]imidazo[1,2-a]pyrimidine-6-carboxamide (1 g, 2.07 mmol) in chloroform (10 mL) was added Dess-Martin Periodinane (1.43 g, 3.38 mmol) at 0-5° C. and stirred for 2 h. After complete consumption of the starting material, the reaction was quenched with saturated cold sodium bicarbonate solution (50 mL), and extracted with ethyl acetate (50 mL×2). The combined organic layer was washed with saturated brine solution, dried over anhydrous Na₂SO₄ and concentrated in vacuo. The crude product was triturated with diethyl ether (20 mL) to afford 2-(4-formylcyclohexyl)-7-isopropoxy-N-[2-oxo-1-[(1S,2R)-2-fluorocyclopropyl]-3-pyridyl] imidazo[1,2-a] pyrimidine-6-carboxamide (0.8 g, 1.51 mmol, 73.10% yield) as yellow solid. LCMS (ES⁺): m/z 482.64 [M+H]⁺

### Step-9:

To a stirred solution of 2-(4-formylcyclohexyl)-7-isopropoxy-N-[2-oxo-1-[(1S,2R)-2-fluorocyclopropyl]-3-pyridyl] imidazo[1,2-a] pyrimidine-6-carboxamide (125 mg, 259.60 mol,) and 1-(5-fluoro-1-methyl-6-(piperidin-4-yl)-1H-indazol-3-yl)dihydropyrimidine-2,4(1H,3H)-dione (119.26 mg, 259.60 μmol, TFA salt) in THF (5 mL) was added triethylamine, 99% (131.34 mg, 1.30 mmol, 180.91 μL) and stirred at 65° C. for 3 h. The reaction mixture was cooled to 0° C. before sodium cyanoborohydride (81.56 mg, 1.30 mmol) was added and stirred at room temperature for 16 h. After complete consumption of the starting material, THF was removed under reduced pressure and water (5 mL) was added to residue and stirred for about 15 min. It was then filtered and the obtained solid was purified by Prep-HPLC to afford 2-[4-[[4-[3-(2,4-dioxohexahydropyrimidin-1-yl)-5-fluoro-1-methyl-indazol-6-yl]-1-piperidyl]methyl]cyclohexyl]-N-[1-[(1S,2R)-2-fluorocyclopropyl]-2-oxo-3-pyridyl]-7-isopropoxy-imidazo[1,2-a]pyrimidine-6-carboxamide (0.05 g, 60.52 μmol, 23.31% yield) as an off white solid.

### Prep-HPLC Method:

Column/dimensions: SUNFIRE c18 (19*150 mm*5 μm)

Mobile phase A: 0.1% FA in WATER

Mobile phase B: Acetonitrile

Gradient (Time/% B): 0/10, 2/10, 8/30, 12/30, 12.10/100

Flow rate: 18 mL/min

Solubility: MeCN+THF

LCMS (ES⁺): m/z 811.58 [M+H]⁺

¹H NMR (400 MHz, DMSO-d₆): δ=10.69 (s, 1H), 10.54 (s, 1H), 9.46 (d, J=4 Hz, 1H), 8.47 (d, J=7.2 Hz, 1H), 7.65-7.58 (m, 2H), 7.45 (d, J=6.8 Hz, 1H), 7.36 (d, J=11.2 Hz, 1H) 6.35 (t, J=7.2 Hz, 1H), 5.65-5.63 (m, 1H), 5.19-4.95 (m, 1H), 3.99 (s, 3H), 3.90 (t, J=6.6 Hz, 2H), 3.48-3.45 (m, 1H), 3.01 (d, 2H), 2.98-2.84 (m, 1H), 2.74 (t, J=6.6 Hz, 2H), 2.65 (m, 1H), 2.20-2.18 (m, 2H), 2.08-1.80 (m, 10H), 1.60-1.53 (8H), 1.47-1.41 (m, 3H), 1.06-1.03 (m, 2H).

### Example 198 was Prepared Substantially Following the Synthesis of Example 113

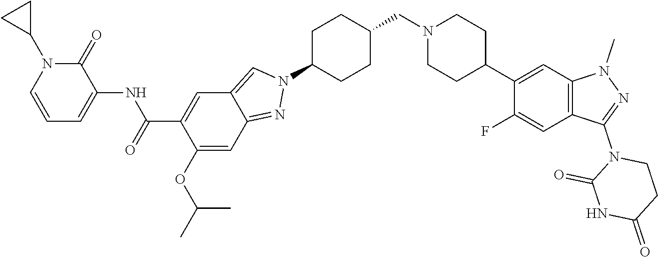 [SMILES: CC(C)Oc1cc2nn([C@H]3CC[C@H](CN4CCC(c5cc6c(cc5F)c(N5CCC(=O)NC5=O)nn6C)CC4)CC3)cc2cc1C(=O)Nc1cccn(C2CC2)c1=O]

### N-(1-cyclopropyl-2-oxo-1,2-dihydropyridin-3-yl)-2-((r,4r)-4-((4-(3-(2,4-dioxotetrahydropyrimidin-1(2H)-yl)-5-fluoro-1-methyl-1H-indazol-6-yl)piperidin-1-yl)methyl)cyclohexyl)-6-isopropoxy-2H-indazole-5-carboxamide

¹H NMR (400 MHz, DMSO-d₆): δ 10.98 (s, 1H), 10.55 (s, 1H), 8.56 (d, J=8.4 Hz, 2H), 8.48 (d, J=6.4 Hz, 1H), 8.39 (s, 1H), 7.61 (d, J=6.0 Hz, 1H), 7.37 (d, J=10.8 Hz, 1H), 7.28 (d, J=7.2 Hz, 1H), 6.27 (t, J=7.2 Hz, 1H), 4.97 (d, J=6.0 Hz, 1H), 4.51 (s, 1H), 4.00 (s, 3H), 3.90 (t, J=6.6 Hz, 2H), 3.51 (s, 1H), 3.00-2.51 (m, 5H), 2.50-2.07 (m, 4H), 2.05-1.67 (m, 11H), 1.52 (d, J=6.0 Hz, 6H), 1.14 (d, J=12.0 Hz, 4H), 0.91 (d, J=6.8 Hz, 2H). LCMS (ES⁺): m/z 792.67 [M+H]⁺.

### Example 199 was Prepared Substantially Following the Synthesis of Example 197

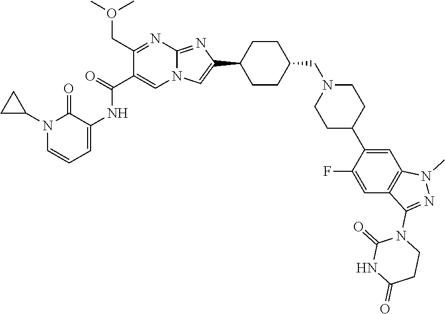

### N-(1-cyclopropyl-2-oxo-1,2-dihydropyridin-3-yl)-2-((1r,4r)-4-((4-(3-(2,4-dioxotetrahydropyrimidin-1(2H)-yl)-5-fluoro-1-methyl-1H-indazol-6-yl)piperidin-1-yl)methyl)cyclohexyl)-7-isopropoxyimidazo[1,2-a]pyrimidine-6-carboxamide

¹H NMR (400 MHz, DMSO-d₆): δ 10.71 (s, 1H), 10.55 (s, 1H), 9.45 (d, J=3.9 Hz, 1H), 8.43 (q, 1H), 7.61 (q, 2H), 7.35 (m, 2H), 6.30 (t, J=7.2 Hz, 1H), 5.62 (m, 1H), 4.00 (s, 3H), 3.90 (t, J=6.7 Hz, 2H), 3.51 (m, 1H), 3.00 (d, J=10.1 Hz, 2H), 2.85 (s, 1H), 2.75 (t, J=6.6 Hz, 2H), 2.58 (s, 1H), 2.21 (q, 2H), 2.06 (q, 6H), 1.93 (d, J=11.2 Hz, 3H), 1.78 (d, J=19.7 Hz, 3H), 1.55 (t, J=3.1 Hz, 6H), 1.42 (q, 3H), 1.05 (q, 2H), 0.92 (t, J=4.6 Hz, 2H). LCMS (ES⁺): m/z 793.59 [M+H]⁺.

### V. Biological Activities

### Assay 1. IRAK4 Degradation Assay, HiBiT Method

Selected compounds were tested in an IRAK4 degradation assay using the HiBiT Method. DC₅₀ values are given in Table 1.

### Materials

Phenol red-free Dulbecco's modified Eagle medium (DMEM) and fetal bovine serum (FBS) were purchased from Gibco (Grand Island, NY, USA). The Nano-Glo® HiBiT Lytic Assay System was purchased from Promega (Madison, WI, USA). Cell culture flasks and 384-well microplates were acquired from VWR (Radnor, PA, USA). The 293Tcell line was engineered by knocking-in a HiBiT fusion tag into the C-terminal of the IRAK4 gene in 293T cells (Synthego, Redwood City, CA, USA).

### IRAK4 Degradation Analysis

IRAK4 degradation was measured via the quantification of luminescent signals using the Nano-Glo® HiBiT Lytic Assay kit. Test compounds were added to 384-well plates in duplicate using an 11-point half-log dilution series, with the highest dose set at 10 mM. 293Tcells expressing HiBiT-tagged IRAK4 were then added into 384-well plates at a cell density of 10,000 cells per well. The plates were kept at 37° C. with 5% CO₂ for 6 hours. Cells that were treated only with DMSO served as the negative control; wells that contained only assay media served as the background control. After the 6-hour incubation, Nano-Glo® HiBiT Lytic Assay reagents were added to the cells. Luminescence was acquired using an EnVision™ Multilabel Reader (PerkinElmer, Santa Clara, CA, USA).

Table 1 shows the activity of selected compounds of this disclosure in the in vitro IRAK assay, wherein each compound number corresponds to the compound numbering set forth in Examples 1-196 described herein.

“+++++” represents a DC₅₀ value less than 10 nM.

“++++” represents a DC₅₀ value of 10 nM-100 nM.

“+++” represents a DC₅₀ value of greater than 100 nM-500 nM.

“++” represents a DC₅₀ value of greater than 500 nM-1000 nM.

“+” represents a DC₅₀ value of greater than 1000 nM

TABLE 1

| Example | DC₅₀ |
|---|---|
| 1 | +++ |
| 2 | ++++ |
| 3 | +++ |
| 4 | ++ |
| 5 | ++ |
| 6 | +++ |
| 7 | +++ |
| 8 | + |
| 9 | ++ |
| 10 | ++++ |
| 11 | ++++ |
| 12 | +++ |
| 13 | ++++ |
| 14 | ++ |
| 15 | ++++ |
| 16 | ++++ |
| 17 | ++++ |
| 18 | ++++ |
| 19 | +++++ |
| 20 | ++++ |
| 21 | ++++ |
| 22 | ++++ |
| 23 | ++++ |
| 24 | ++++ |
| 25 | +++ |
| 26 | +++ |
| 27 | ++++ |
| 28 | ++++ |
| 29 | ++++ |
| 30 | +++++ |
| 31 | ++++ |
| 32 | +++ |
| 33 | +++ |
| 34 | +++ |
| 35 | ++++ |
| 36 | +++ |
| 37 | ++++ |
| 38 | +++++ |
| 39 | ++++ |
| 40 | +++ |
| 41 | ++++ |
| 42 | +++ |
| 43 | ++++ |
| 44 | ++++ |
| 45 | +++ |
| 46 | ++++ |
| 47 | +++++ |
| 48 | +++++ |
| 49 | +++++ |
| 50 | +++++ |
| 51 | +++++ |
| 52 | +++++ |
| 53 | ++++ |
| 54 | +++ |
| 55 | +++++ |
| 56 | +++++ |
| 57 | +++++ |
| 58 | ++++ |
| 59 | ++++ |
| 60 | +++ |
| 61 | +++ |
| 62 | +++++ |
| 63 | ++++ |
| 64 | ++ |
| 65 | ++++ |
| 66 | ++++ |
| 67 | +++++ |
| 68 | ++++ |
| 69 | +++++ |
| 70 | +++++ |
| 71 | ++++ |
| 72 | ++++ |
| 73 | +++++ |
| 74 | ++++ |
| 75 | ++++ |
| 76 | ++++ |
| 77 | ++++ |
| 78 | ++++ |
| 79 | ++++ |
| 80 | ++++ |
| 81 | ++++ |
| 82 | ++++ |
| 83 | ++++ |
| 84 | ++++ |
| 85 | ++++ |
| 86 | ++++ |
| 87 | +++++ |
| 88 | ++++ |
| 89 | +++++ |
| 90 | ++++ |
| 91 | ++++ |
| 92 | ++++ |
| 93 | +++ |
| 94 | +++ |
| 95 | +++ |
| 96 | ++++ |
| 97 | ++++ |
| 98 | ++++ |
| 99 | ++++ |
| 100 | ++++ |
| 101 | ++++ |
| 102 | ++++ |
| 103 | ++++ |
| 104 | +++++ |
| 105 | ++++ |
| 106 | +++ |
| 107 | ++++ |
| 108 | ++++ |
| 109 | ++++ |
| 110 | ++++ |
| 111 | ++++ |
| 112 | ++++ |
| 113 | +++++ |
| 114 | ++++ |
| 115 | +++++ |
| 116 | +++++ |
| 117 | +++++ |
| 118 | ++++ |
| 119 | +++++ |
| 120 | +++++ |
| 121 | +++++ |
| 122 | +++++ |
| 123 | +++++ |
| 124 | +++++ |
| 125 | +++++ |
| 126 | +++++ |
| 127 | ++++ |
| 128 | +++++ |
| 129 | +++++ |
| 130 | +++++ |
| 131 | ++++ |
| 132 | +++++ |
| 133 | +++++ |
| 134 | +++++ |
| 135 | +++++ |
| 136 | +++++ |
| 137 | +++++ |
| 138 | +++++ |
| 139 | +++++ |
| 140 | +++++ |
| 141 | +++++ |
| 142 | +++++ |
| 143 | ++++ |
| 144 | +++++ |
| 145 | +++++ |
| 146 | +++++ |
| 147 | +++++ |
| 148 | ++++ |
| 149 | +++++ |
| 150 | +++++ |
| 151 | +++++ |
| 152 | +++++ |
| 153 | ++++ |
| 154 | +++++ |
| 155 | ++++ |
| 156 | ++++ |
| 157 | +++++ |
| 158 | +++++ |
| 159 | +++++ |
| 160 | +++++ |
| 161 | ++++ |
| 162 | ++++ |
| 163 | +++++ |
| 164 | ++++ |
| 165 | ++++ |
| 166 | ++++ |
| 167 | +++++ |
| 168 | ++++ |
| 169 | +++++ |
| 170 | ++++ |
| 171 | +++++ |
| 172 | ++++ |
| 173 | +++++ |
| 174 | ++++ |
| 175 | +++++ |
| 176 | +++++ |
| 177 | +++++ |
| 178 | +++++ |
| 179 | +++++ |
| 180 | ++++ |
| 181 | +++++ |
| 182 | +++++ |
| 183 | +++++ |
| 184 | +++++ |
| 185 | +++++ |
| 186 | +++++ |
| 187 | +++++ |
| 188 | +++++ |
| 189 | +++++ |
| 190 | ++++ |
| 191 | +++++ |
| 192 | +++++ |
| 193 | ++++ |
| 194 | ++++ |
| 195 | ++++ |
| 196 | +++++ |
| 197 | +++++ |
| 198 | +++++ |

### Assay 2. IKZF1 Degradation Assay, HiBiT Method

Selected compounds of this disclosure were tested in an IKZF1 degradation assay using the HiBiT Method. The results are set forth in Table 2.

### Materials

RPMI no-phenol red medium and fetal bovine serum (FBS) were purchased from Gibco (Grand Island, NY, USA). Nano-Glo® HiBiT Lytic Assay System was purchased from Promega (Madison, WI, USA). NCIH929 (HiBiT-IKZF1) cell line was generated in house, endogenously expressing IKZF1 with HiBiT fusion tag via CRISPR from NCIH929 cells (ATCC CRL-9068 Manassas, VA, USA). Cell culture flasks and 384-well microplates were acquired from VWR (Radnor, PA, USA).

### IKZF1 Degradation Analysis

IKZF1 degradation was determined based on quantification of luminescent signal using Nano-Glo® HiBiT Lytic Assay kit. Test compounds of this disclosure, and pomalidomide (POMALYST®, Bristol Myers Squibb, New York, NY, USA) as a positive control, were added to the 384-well plate from a top concentration of 10 μM with 11 points, half-log titration in duplicates. NCIH929 cells expressing HiBiT-tagged IKZF1 were added into 384-well plates in RPMI medium containing 10% FBS and 0.05 mM 2-mercaptoethanol at a cell density of 15000 cells per well. The plates were kept at 37° C. with 5% CO₂ for 6 hours. Cells treated in the absence of the test compound were the negative control and wells containing media only were the positive control. After 6-hour incubation, Nano-Glo® HiBiT Lytic Assay reagents were added to the designated wells. Luminescence was acquired on EnVision® Multilabel Reader (PerkinElmer, Santa Clara, CA, USA).

Table 2 shows the activity of selected compounds of this disclosure in the in vitro IKZF1 assay, wherein each compound number corresponds to the compound numbering set forth in Examples 1-197 described herein.

TABLE 2

| Example | DC₅₀ (nM) |
|---|---|
| 23 | N/A |
| 31 | N/A |
| 38 | N/A |
| 41 | N/A |
| 47 | N/A |
| 48 | N/A |
| 49 | N/A |
| 50 | N/A |
| 52 | N/A |
| 53 | N/A |
| 55 | N/A |
| 68 | N/A |
| 69 | N/A |
| 70 | N/A |
| 71 | N/A |
| 73 | N/A |
| 81 | N/A |
| 87 | N/A |
| 96 | N/A |
| 98 | N/A |
| 104 | N/A |
| 105 | N/A |
| 108 | N/A |
| 113 | N/A |
| 114 | N/A |
| 115 | N/A |
| 120 | N/A |
| 121 | N/A |
| 122 | N/A |
| 123 | N/A |
| 125 | N/A |
| 128 | N/A |
| 129 | N/A |
| 142 | N/A |
| 147 | N/A |
| 153 | N/A |
| 159 | N/A |
| 162 | N/A |

“N/A” means no significant activity detected at the highest concentration tested.

No significant effect on IKZF1 protein level was observed following 6 hours treatment of NCIH929.11 cells with test compounds of this disclosure listed in Table 2 at concentrations up to 10 μM while positive control, pomalidomide, induced 90% degradation of IKZF1 with a DC₅₀ of 44 nM at 6 hours.

### Assay 3. Pharmacokinetics (PK) in Male Beagle Dogs

The pharmacokinetic (PK) profile in plasma of two compounds of the present invention was determined in male beagle dogs following single dose IV (5 mg/kg) and PO (10 mg/kg) administration. This study was performed under non-GLP conditions, and unless otherwise stated, all analytical reagents were standard laboratory reagent grade.

Male beagle dogs were housed individually and maintained in a controlled environment. Pedigree® standard dog chow (Pedigree India Private Ltd, Telangana, India) was provided once daily. Drinking water was available ad libitum. Environmental controls for the animal room were set to maintain a temperature range of 22-25° C., relative humidity range of 40-70%, and a 12-hour light/12-hour dark cycle. Normal healthy animals weighing 10±1 kg, certified by the attending veterinarian, were selected and acclimatized for a minimum of three days prior to initiation of study. Dogs were identified by chip number inserted at the neck region. The study protocol was reviewed and approved by the Institutional Animal Ethics Committee (IAEC).

2-[4-[[4-[1-(2,6-dioxo-3-piperidyl)-3-methyl-2-oxo-benzimidazol-5-yl]-1-piperidyl]methyl]cyclohexyl]-7-isopropoxy-N-pyrazolo[1,5-a]pyrimidin-3-yl-imidazo[1,2-a]pyridine-6-carboxamide, as described in Example 48 (“Compound 48”), and 2-((1R,4S)-4-((4-(1-(2,6-dioxopiperidin-3-yl)-3-methyl-2-oxo-2,3-dihydro-1H-benzo[d]imidazol-5-yl)piperidin-1-yl)methyl)cyclohexyl)-N-(1-((1S,2R)-2-fluorocyclopropyl)-2-oxo-1,2-dihydropyridin-3-yl)-7-isopropoxyimidazo[1,2-a]pyridine-6-carboxamide, as described in Example 169 (“Compound 169”), were each formulated by dissolving the compound in polyethylene glycol (PEG400) (10%) with stirring, followed by the slow addition of 11.11% 2-Hydroxypropyl-beta-cyclodextrin (HPβCD) (90%) in water. Vehicle dose was PEG400 (10%) and 11.11% HP-β-CD (90%) in water. Formulations and vehicle were freshly prepared on the day of dosing and stored at room temperature until used.

All animals were weighed prior to drug administration. The animals were divided into intravenous (IV) group and per os (PO) group for each compound tested, with 3 animals in each group. The vehicle dosing group also included IV and PO groups, making a total of 6 groups of 3 animals each, for 18 animals total. The animals were restrained physically in sternal recumbently, on an examination table. The IV dose was administered by intravenous bolus injection into the cephalic vein and the PO dose was administered via gastric tube. The dosing volume was 1 mL/kg for IV groups and 2 mL/kg for PO groups. The dosing concentration was 5 mg/mL for both IV and PO groups respectively. The animals were fasted overnight and food was provided 4 hours post dose. After administration, animals were observed at regular intervals for any sign of illness or reaction to treatment. Apart from this, animals were observed twice daily for any clinical signs, if any, and noted accordingly.

From the vehicle treatment group, whole blood was collected for peripheral blood mononuclear cells (PBMC) isolation at Pre-dose, and at 2, 8, 24, 48, 72 and 96 hour timepoints. For both tested compounds IV and PO dosing groups, whole blood was collected for plasma isolation at Pre-dose, and 0.033, 0.083, 0.167, 0.33, 1, 2, 4, 6, 8, 24, 48, 72 and 96 hour timepoints. The anti-coagulant solution used was 6% (v/v) Sodium citrate (200 mM, pH 4.79) (pre-chilled tubes). The whole blood from both IV and PO compound treatment dosing groups collected at Pre-dose, 2, 8, 24, 48, 72 and 96 hours were subjected to PBMC isolation.

The whole blood collected at specified timepoints above designated for PBMC isolation was immediately subjected to PBMC isolation procedure, as described in Assay 4 below.

For sample preparation, approximately 1-2 mL of whole blood was drawn from the peripheral vein and collected in labeled tubes containing lithium heparin, then stored on ice. The blood samples were centrifuged within 15 minutes to separate plasma at 1540 g at 4° C. for 10 minutes. The plasma was separated and transferred to pre-labeled microcentrifuge tubes and immediately frozen at −80±10° C. until bioanalysis. Samples were identified by test item, group, animal number, and collection time point. For collection of blood samples, a time window of ±2 minutes for 0.5 hour and +5 minutes for 1 to 8 hours and +15 minutes for 24 hours, was allowed and was not considered as a deviation.

All samples were analyzed by ExionLC™ AD high-pressure liquid chromatography (HPLC) system (AB Sciex LLC, Framingham, MA) followed by tandem mass spectroscopy analysis (MS/MS) with SCIEX Triple Quad™ 4500 (AB Sciex LLC, Framingham, MA). The samples were resolved on a Kinetex® 5 μm EVO C18 column (50×4.6 mm) (Phenomenex, Inc., Torrance, CA) with 10 mM ammonium acetate with 0.1% Formic acid in Milli-Q® water (EMD Millipore, Burlington, MA) as an aqueous (A) mobile phase and 100% methanol as an organic (B) mobile phase. The flow rate was set at 1 mL/min. The LC gradient program included initial conditions of 95% A at 0 minutes, with switch to 5% A at 1 minute and hold until 2.5 minutes before returning to initial conditions of 95% A at 2.6 minutes with a hold till 3.0 minutes at 95% A.

A positive electrospray ionization (ESI) method was used for detecting analytes and internal standard by mass spectroscopy. The selective reaction monitoring (SRM) conditions for Compound 48 were Q1 m/z 773.1, Q3 m/z 389, declustering potential (DP) 100 V and collision energy (CE) 57 eV. The SRM conditions for Compound 169 were Q1 m/z 807.5, Q3 m/z 765.6, DP 80 V and CE of 49 eV. Other MS/MS conditions for TAs and internal standard included Collision Cell Exit Potential (CXP) 10, Collision Gas (CAD) MEDIUM, Curtain Gas (CUR) 40, Nebulizer Gas (GS1) 55, Heater Gas (GS2) 65, Ion spray voltage (V) 5500, Temperature (TEM) 550 and Interface Heater (IHE) ON.

For sample bioanalysis, an LC-MS/MS method for analyzing plasma samples was developed as per the bioanalytical guidelines. One set of nine calibration standards were run before the sample batch. The calibration range was 1-1000 ng/mL for all samples. Calibration standards were acceptable if the back-calculated concentrations did not deviate by more than 20%. In case the lowest standard is excluded the reported measured concentration of the study samples must be above the next lowest acceptable standard. If the highest standard is excluded, the reported highest calibration standard is the second highest standard. If the study sample concentration was above the upper limit of quantitation (ULOQ), the study samples were diluted with blank matrix, processed, and analyzed.

Quality control (QC) samples were prepared at a minimum of three concentrations, i.e., LQC (not more than 5 times to that of lowest standard concentration), HQC (not less than 75% of the highest standard concentration), and MQC (between the low and high concentration). A minimum of 6 QC samples (three concentrations in duplicate) and one set of QCs (LQC, MQC, and HQC) samples were analyzed before and after the sample batch. The back-calculated concentration of each QC was within 20% of the nominal concentration if no pre-study validation done. No QC level was completely discarded. To accept an analytical run, at least two thirds of the calibration standards and QC met the stated acceptance criteria.

Pharmacokinetic parameters like AUC₀₋₂₄, AUCₗₐₛₜ, AUC_{0-inf}, AUC_{Extra} (%), Cₘₐₓ, C_{max_D}, T_{1/2}, Tₘₐₓ, MRT, C₀, Vd, Cl, % F were calculated for each animal by non-compartmental model with Phoenix® software version 8.1 (Certara, Princeton, NJ, USA). All values of the calculated parameters are reported as value ±SD to four significant figures.

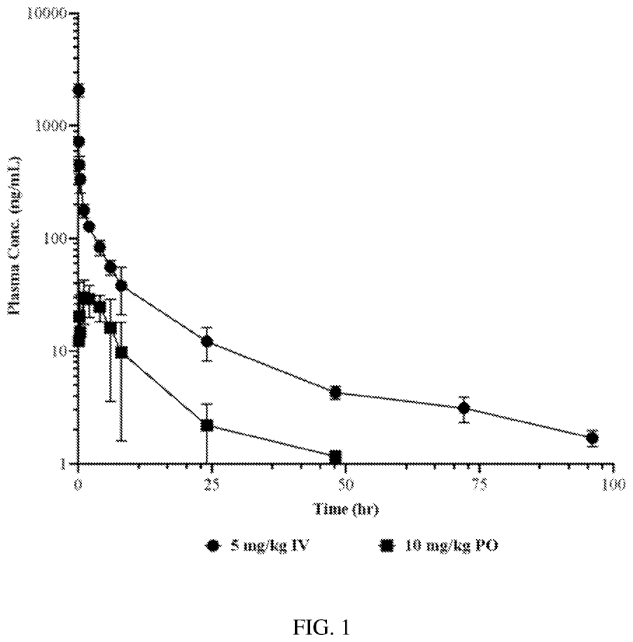

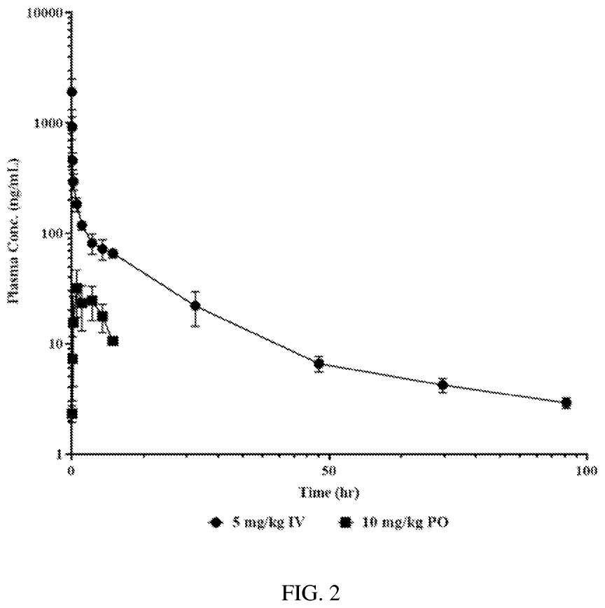

The results of this PK study in male beagle dogs for Compound 48 are shown in Table 3 and Table 4 below and FIG. 1, and the results for Compound 169 are shown in Table 5 and Table 6 below and FIG. 2.

TABLE 3
Calculated pharmacokinetic parameters following 5
mg/kg IV dosing of Compound 48 in Male Beagle Dogs

| | 5 mg/kg IV | | | | |
|---|---|---|---|---|---|
| | C₀ | T_{1/2} | Vdss | Cl | AUCₗₐₛₜ |
| Values | (ng/mL) | (h) | (L/kg) | (ml/min/kg) | (h*ng/ml) |
| Mean | 4180 | 34.47 | 58.45 | 46.32 | 1719 |
| SD | 796.7 | 15.95 | 14.65 | 3.904 | 208.8 |

TABLE 4
Calculated pharmacokinetic parameters following 10
mg/kg PO dosing of Compound 48 in Male Beagle Dogs

| | 10 mg/kg PO | | |
|---|---|---|---|
| | Cₘₐₓ | AUCₗₐₛₜ | |
| Values | (ng/mL) | (h*ng/ml) | % F |
| Mean | 33.88 | 241.5 | 7.023 |
| SD | 11.50 | 158.9 | 4.621 |

TABLE 5
Calculated pharmacokinetic parameters following 5
mg/kg IV dosing of Compound 169 in Male Beagle Dogs

| | 5 mg/kg IV | | | | |
|---|---|---|---|---|---|
| | C₀ | T_{1/2} | Vdss | Cl | AUCₗₐₛₜ |
| Values | (ng/ml) | (h) | (L/kg) | (ml/min/kg) | (h*ng/ml) |
| Mean | 3161 | 42.48 | 57.97 | 35.04 | 2211 |
| SD | 1274 | 10.05 | 12.71 | 3.046 | 216.6 |

TABLE 6
Calculated pharmacokinetic parameters following 10
mg/kg PO dosing of Compound 169 in Male Beagle Dogs

| | 10 mg/kg PO | | |
|---|---|---|---|
| | Cₘₐₓ | AUCₗₐₛₜ | |
| Values | (ng/ml) | (h*ng/ml) | % F |
| Mean | 32.22 | 472.5 | 10.69 |
| SD | 14.38 | 53.78 | 1.216 |

### Assay 4. Pharmacodynamics (PD) in Male Beagle Dogs

Pharmacodynamics (PD) biomarker analyses of male beagle dog PMBCs from the whole blood timepoint samples collected in Assay 3 were performed.

For isolation of PBMCs, the whole blood timepoint samples described in Assay 3 above were processed immediately to ensure high viability of PBMCs. All the samples were processed at 18° C. to 20° C. The freshly collected whole blood (2 mL) was mixed with an equal volume of phosphate buffered saline. This mixture was inverted several times to ensure thorough mixing. The Histopaque® (Catalog no. 10771, Sigma-Aldrich, St. Louis, MO) bottle was inverted several times to ensure uniformity of the medium. A sterile syringe was used to draw 2 mL of Histopaque® medium and added to a sterile centrifuge tube. The whole blood-PBS sample (4 ml) was carefully layered on top of the Histopaque® medium, without mixing. The tube was centrifuge at 600 g for 30 to 40 min at 18° C. to 20° C. with the brakes turned off. The upper layer containing plasma and platelets was withdrawn slowly using a sterile pipette, leaving the PBMC cell layer undisturbed at the interface. The PBMC layer was transferred to a sterile centrifuge tube using a sterile pipette. The PBMCs were washed with 3 volumes (˜6 mL) of Hank's balanced salt solution (HBSS) (Gibco, Grand Island, NY) in the centrifuge tube. The cells were suspended by gently drawing them in and out of a pipette. The tubes were centrifuged at 250 g for 10 to 15 minutes at 18° C. to 20° C. and the supernatant discarded. The PBMCs were then resuspended in 6 to 8 ml of HBSS. For cell counting, the cell suspension was diluted 1:1 with Trypan Blue dye and mixed well. The cell numbers (live and dead) were counted, and cell viability calculated. The cell numbers and cell viability of each PBMC sample were recorded. The tube was centrifuged again at 500 g for 10 min at 18° C. to 20° C. The supernatant was removed, and the cell pellet was frozen at −80° C.

The frozen dog PBMC cell pellets were homogenized in two pellet volumes of lysis buffer (100 mM triethylammonium bicarbonate (TEAB), 4% SDS, 1× Roche protease inhibitor cocktail) by sonicating at 150 watt for 10 seconds in a 4° C. water bath using an E220 focused-ultrasonicator (Covaris, Woburn, MA, USA). Lysates were then clarified by centrifugation at 18,000 g for 20 minutes at 4° C. and protein concentrations were determined by bicinchoninic acid (BCA) assay (ThermoFisher Scientific, Waltham, MA, USA).

For protein digestion, an equal amount of protein was taken from each sample. Proteins were reduced with 10 mM DTT for 10 minutes at 70° C. and alkylated with 25 mM iodoacetamide for 30 minutes in the dark. Tryptic digestion was performed with S-Trap™ method (Protifi, Farmingdale, NY, USA) overnight at 37° C. In brief, samples were mixed with 90% MeOH in 100 mM TEAB containing 10% (w/w) trypsin and loaded onto an in-house packed S-Trap microcolumns. Samples were washed with 90% MeOH in 100 mM TEAB, and digested in 100 mM TEAB for 16 hours at 37° C. The resulting peptides were spiked with five heavy-labeled IRAK4 peptides (Vivitide, LLC, Gardner, MA, USA) at 2 fmol each (column loading), desalted using C18 StageTip, SpeedVac dried, and kept at −80 C until nanoLC-MS/MS analysis.

Peptide analysis was performed on a nanoLC-MS/MS platform composed of a Q Exactive HF mass spectrometer coupled to an EASY-nLC 1200 (ThermoFisher Scientific). Peptides were separated on an EASY-Spray™ C18 column (50 cm×75 am, 2 am) (ThermoFisher Scientific) at 50° C. Mobile phases were 0.1% formic acid in water (A) and 80% acetonitrile, 0.1% formic acid (B). Peptides were eluted with a 100-min method over an effective 80 min gradient from 200 to 45% B at a flow rate of 275 nL/min. Peptides were ionized with a spray voltage of 1,800 V. Mass spectrometric data were acquired at parallel reaction monitoring (PRM) mode including five pairs of light and heavy IRAK4 peptides (Table 7 below). A 30,000 resolution with 5e5 AGC and 150 ms maximum IT was set for MS2, and isolation window was set at 1.0 Th. (N)CE was optimized using the heavy-labeled peptides.

TABLE 7
Five pairs of light and heavy dog IRAK4 peptides

| | | Charge | | |
|---|---|---|---|---|
| Peptides | Mass [m/z] | State [z] | Polarity | (N)CE |
| Light1_NVTNNFDERPISLGGNK | 625.6500 | 3 | Positive | 26 |
| (SEQ ID NO: 1) | | | | |
| Heavy1_NVTNNFDERPISLGGNK | 628.3214 | 3 | Positive | 26 |
| (SEQ ID NO: 1) | | | | |
| Light2_VSDFGLAR | 432.7323 | 2 | Positive | 18 |
| (SEQ ID NO: 2) | | | | |
| Heavy2_VSDFGLAR | 437.7365 | 2 | Positive | 18 |
| (SEQ ID NO: 2) | | | | |
| Light3_SANILLDEDFTAK | 718.8670 | 2 | Positive | 21 |
| (SEQ ID NO: 3) | | | | |
| Heavy3_SANILLDEDFTAK | 722.8741 | 2 | Positive | 21 |
| (SEQ ID NO: 3) | | | | |
| Light4_FHSFSFYELK | 435.5485 | 3 | Positive | 18 |
| (SEQ ID NO: 4) | | | | |
| Heavy4_FHSFSFYELK | 438.2199 | 3 | Positive | 18 |
| (SEQ ID NO: 4) | | | | |
| Light5_IVGTTAYMAPEALR | 746.8951 | 2 | Positive | 25 |
| (SEQ ID NO: 5) | | | | |
| Heavy5_IVGTTAYMAPEALR | 751.8992 | 2 | Positive | 25 |
| (SEQ ID NO: 5) | | | | |

Peptide quantification was processed in Skyline. At least five transitions were selected and summed to generate peak intensity. L/H intensity ratio of each peptide was normalized to the mean of controls and all peptides were averaged for each animal to get the IRAK4 relative abundance levels.

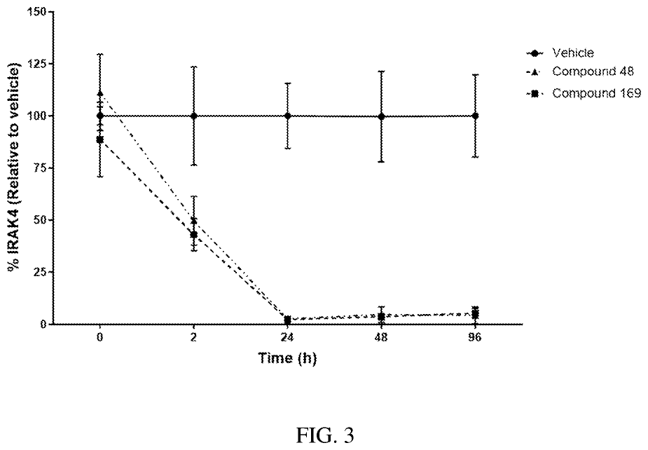

The pharmacodynamics analyses results for IRAK4 quantitation are shown in Table 8 below and in FIG. 3.

TABLE 8

IRAK4 change from vehicle

| Time (h) | Compound 48 | Compound 169 |
|---|---|---|
| 0 | +12% | −11% |
| 2 | −51% | −57% |
| 24 | −97% | −97% |
| 48 | −95% | −96% |
| 96 | −95% | −95% |

### Assay 5. Pharmacokinetics in Male Cynomolgus Monkeys

The pharmacokinetic (PK) profile in plasma of two compounds of the present invention was determined in male cynomolgus monkeys following single dose IV (5 mg/kg) and PO (10 mg/kg) administration. This study was performed under non-GLP conditions, and unless otherwise stated, all analytical reagents were standard laboratory reagent grade. The study was conducted in accordance with IACUC guidelines in compliance with the Animal Welfare Act, the Guide for the Care and Use of Laboratory Animals.

Male cynomolgus monkeys, non-naïve, are acceptable species to support PK studies for compounds intended for use in humans. The animals were supplied by Hainan Jingang Laboratory Animal Co. Ltd (Nayangxintan Fucheng Town, Qiongshan District, Haikou Hainan Province, P.R. China) or another qualified source. During in-life, the animals were individually housed in stainless-steel mesh cages that are in accordance with the National Research Council “Guide for the Care and Use of Laboratory Animals”. Animals were fed twice daily with approximately 120 g of certified monkey diet. These amounts were adjusted as necessary based on food consumption of the group or an individual body weight changes of the group or an individual and/or changes in the certified diet. In addition, animals received fruit daily as nutritional enrichment and reverse osmosis (RO) water was available to all animals, ad libitum. RO water was analyzed every three months and every batch of feed was analyzed before using. Feed and water analyses records were maintained in the facility records. The room(s) were controlled and monitored for relative humidity range of 40-70% (any excursion from this range for more than 3 hours was documented as a deviation) and temperature range of 18-26° C. (any excursion from this range was documented as a deviation) with 10 to 20 air changes/hour. The room was on a 12-hour light/dark cycle except when interruptions were necessitated by study activities. Normal healthy animals weighing >2 kg and age ≥2 years old were selected for studies. The monkeys were identified by a unique skin tattoo on chest.

2-[4-[[4-[1-(2,6-dioxo-3-piperidyl)-3-methyl-2-oxo-benzimidazol-5-yl]-1-piperidyl]methyl]cyclohexyl]-7-isopropoxy-N-pyrazolo[1,5-a]pyrimidin-3-yl-imidazo[1,2-a]pyridine-6-carboxamide, as described in Example 48 (“Compound 48”), and 2-((1R,4S)-4-((4-(1-(2,6-dioxopiperidin-3-yl)-3-methyl-2-oxo-2,3-dihydro-1H-benzo[d]imidazol-5-yl)piperidin-1-yl)methyl)cyclohexyl)-N-(1-((1S,2R)-2-fluorocyclopropyl)-2-oxo-1,2-dihydropyridin-3-yl)-7-isopropoxyimidazo[1,2-a]pyridine-6-carboxamide, as described in Example 169 (“Compound 169”), were each formulated by dissolving the compound in polyethylene glycol (PEG400) (10%) with stirring, followed by the slow addition of 11.11% 2-Hydroxypropyl-beta-cyclodextrin (HPβCD) (90%) in water. Vehicle dose was PEG400 (10%) and 11.11% HP-β-CD (90%) in water. Formulations and vehicle were freshly prepared on of the day of dosing and stored at room temperature until used.

All animals were weighed prior to drug administration. The animals were divided into intravenous (IV) group and per os (PO) group for each compound tested, with 3 animals in each group. The vehicle dosing group included IV and PO groups, making a total of 6 groups of 3 animals each, for 18 animals total. The IV dose was administered by intravenous bolus injection into the cephalic or saphenous vein. The vein used for the dosing was not used for blood sample collection for the first 4 hours post dose. The PO dose was administered by nasogastric tube. The nasogastric PO doses were flushed using 3 mL of vehicle (approximately 3 times volume of nasogastric tube). All tubes were of equal size and not variable between animals and cut to equal length so that the flush volume was comparable. The dosing volume was 2.5 mL/kg for the IV groups and 5 mL/kg for the PO groups. The dosing concentration was 2 mg/mL for both IV and PO groups. Feeding conditions were overnight fasting for PO group but not for IV group. After administration, animals were observed at regular intervals for any sign of illness or reaction to treatment. Apart from this, animals were observed twice daily for any clinical signs, if any and noted accordingly.

For the vehicle treatment group, biomarker analysis was conducted on whole blood and in PBMCs at 2, 8, 24, 48, 72 and 96 h timepoints. For both compounds tested, whole blood was collected for plasma isolation from the IV dosing group at 0.033, 0.083, 0.167, 0.33, 1, 2, 4, 6, 8, 24, 48, 72, and 96 hours timepoints and from the PO dosing group at 0.167, 0.33, 1, 2, 4, 6, 8, 24, 48, 72, and 96 hours timepoints. From the compound treatment groups, biomarker analysis was conducted on whole blood and in PBMCs at 2, 8, 24, 48, 72 and 96 hours timepoints.

The whole blood collected at specified timepoints above designated for PBMC isolation was immediately subjected to PBMC isolation procedure, as described in Assay 6 below.

For sample preparation, 0.5 mL of blood was utilized for PK bioanalysis; and 2 mL for PBMC isolation and IRAK4 analysis. The anti-coagulant solution was ethylenediaminetetraacetic acid dipotassium salt (K2-EDTA) for plasma PK samples and whole blood designated for PBMC isolation.

For PK sample bioanalysis, approximately 0.5 mL of whole blood was collected into labeled tubes Eppendorf® Protein LoBind tubes (Eppendorf, Enfield, CT, USA) containing 5 μL of 0.5 M K2-EDTA on ice. Samples were centrifuged at 3,200 g for 10 minutes at 2 to 8° C. within one hour of collection. The resulting 0.2 mL of plasma samples were transferred into labeled Eppendorf® Protein LoBind tubes containing 2 μL 20% Triton X-100 and were stored at −80° C. until bioanalysis. The samples were divided into two aliquots of 0.1 mL each, one was subjected to bioanalysis, and the other was saved as back up.

For PK sample bioanalysis, all samples were analyzed by high-pressure liquid chromatography (HPLC) followed by tandem mass spectroscopy analysis (MS/MS) with SCIEX Triple Quad™ 6500+(Sciex, Framingham, MA, USA). The samples were resolved on XSELECT CSH C18 2.5 m Column XP (2.1×50 mm) (Waters, Milford, MA, USA) with 2 mM ammonium acetate in water/ACN (95:5 v/v) as an aqueous (A) mobile phase and 2 mM ammonium acetate water/ACN (5:95 v/v) as an organic (B) phase. The flow rate was set at 0.6 mL/min. The LC gradient program included initial conditions of 95% A at 0 min, hold till 0.2 min. with switch to 5% A at 1.2 min and hold until 1.4 min before returning to initial conditions of 95% A at 1.41 with a hold till 1.6 min at 95% A. The column temperature was maintained at 50.0° C. A positive electrospray ionization (ESI) method was used for detecting analytes and internal standard by mass spectroscopy. The selection reaction monitoring (SRM) conditions for Compound 48 were Q1 m/z 773.60, Q3 m/z 389.20, declustering potential (DP) 100 V and collision energy (CE) 55 eV. The SRM conditions for Compound 169 were Q1 m/z 807.60, Q3 m/z 423.30, delustering potential (DP) 110 V and collision energy (CE) 55 eV. The SRM conditions for verapamil were Q1 m/z 455.20, Q3 m/z 164.90. The SRM conditions for labetalol were Q1 m/z 329.10, Q3 m/z 161.90. Other MS/MS conditions for TAs and internal standard included Collision Cell Exit Potential (CXP)16, Collision Gas (CAD) 10, Curtain Gas (CUR) 40, Nebulizer Gas (GS1) 50, Heater Gas (GS2) 50, Ion spray voltage (V) 5500, Temperature (TEM) 550, and Interface Heater (IHE) ON.

For plasma sample bioanalysis, all plasma samples and blank matrix were mixed with 20% Triton X-100 in water, the final concentration of Triton X-100 was 0.2% at the source of in-life work. A 20 μL aliquot of all samples, i.e., unknown, calibration standard, quality control and dilution quality control (if any), single blank, and double blank samples, were quenched with 400 μL of internal standard 1 (IS1) in a 96 well plate. The IS1 contains labetalol, tolbutamide, verapamil, dexamethasone, glyburide & celecoxib at concentration of 100 ng/mL each in acetonitrile (ACN). The double blank sample was quenched with 400 μL of ACN. The plate was vortexed for 10 minutes at 800 rpm and then centrifuged for 15 minutes at 3220 g at 4° C. A 50 μL aliquot of supernatant was then transferred to another clean 96-well plate and centrifuged for 5 minutes at 3220 g at 4° C. and samples were directly subjected to LC-MS/MS analysis.

For dose formulation concentration verification, a LC-UV method was developed with a calibration curve consisting of 6 calibration standards. The acceptance criteria for an analytical run were, 5 of 6 calibration standards should be within ±20% of nominal values by using LC-UV method.

For PK sample bioanalysis, an LC-MS/MS method for analyzing the plasma samples was developed as per the bioanalytical guidelines for Non-GLP compliance. The method calibration curve consisting of 6 calibration standards, with a calibration range of 1.00-3000 ng/mL. A calibration curve with at least 6 non-zero calibration standards were applied for the method including lower limit of quantitation (LLOQ). The linearity for ≥75% calibration standards are within ±20% of their nominal values in plasma. If the endpoints, such as LLOQ and ULOQ, on the calibration curve are eliminated, the calibration curve were truncated. The truncated calibration curve should consist of at least 75% of the initial STDs. A set of QCs consisting of low, middle, and high concentrations were applied for the method. The accuracy of ≥67% QCs is back calculated to within ±20% of their nominal values for plasma. For the specificity acceptance criteria, the mean calculated concentration in the single blank matrix should be ≤50% LLOQ. The sensitivity acceptance criteria were set as per the biological matrix, for plasma ≤2 ng/mL, and ≤4 ng/mL for matrix other than plasma.

Pharmacokinetic parameters like AUC₀₋₂₄, AUCₗₐₛₜ, AUC_{0-if}, AUC_{Extra} (%), Cₘₐₓ, C_{max_D}, T_{1/2}, Tₘₐₓ, MRT, C₀, Vd, Cl, and % F were calculated for individual animal by non-compartmental model with Phoenix WinNonlin 6.3 software program. All values of the calculated parameters are reported as value ±SD to four significant figures.

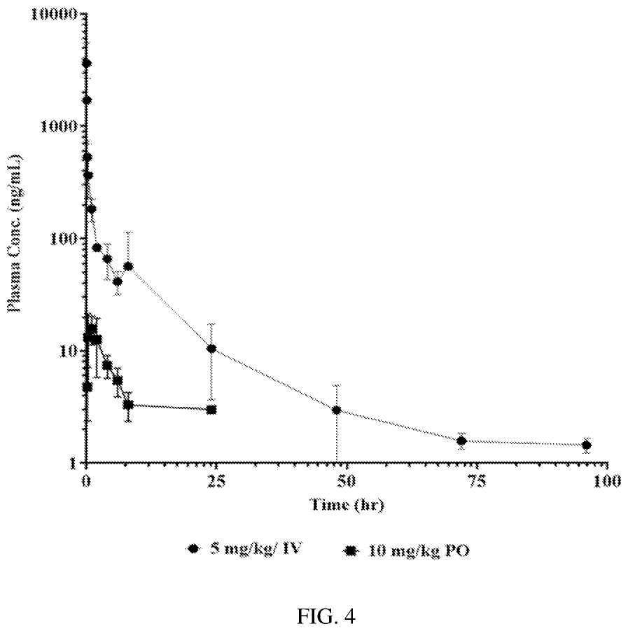

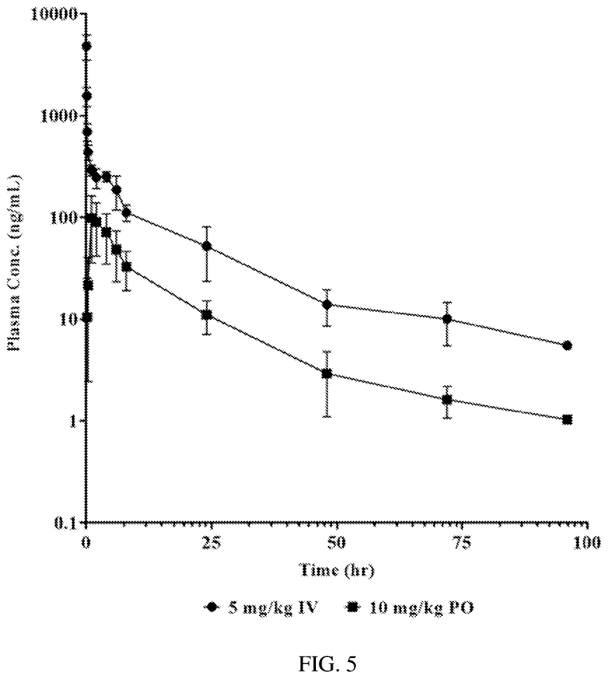

The results of this PK study in male cynomolgus monkeys for Compound 48 are shown in Table 9 and Table 10 below and FIG. 4, and the results for Compound 169 are shown in Table 11 and Table 12 below and FIG. 5.

TABLE 9
Calculated pharmacokinetic parameters following 5 mg/kg
IV dosing of Compound 48 in Male Cynomolgus Monkeys

| | 5 mg/kg IV | | | | |
|---|---|---|---|---|---|
| | C₀ | T_{1/2} | Vdss | Cl | AUCₗₐₛₜ |
| Values | ng/ml | (h) | (L/kg) | (ml/min/kg) | (h*ng/mL) |
| Mean | 5973 | 44.2 | 70.3 | 48.6 | 1740 |
| SD | 3013 | 53.6 | 77.7 | 13.7 | 662 |

TABLE 10
Calculated pharmacokinetic parameters following 10 mg/kg
PO dosing of Compound 48 in Male Cynomolgus Monkeys

| | 10 mg/kg PO | | |
|---|---|---|---|
| | Cₘₐₓ | AUCₗₐₛₜ | |
| Values | (ng/ml) | (h*ng/mL) | % F |
| Mean | 18.3 | 84.3 | 2.42 |
| SD | 4.53 | 45.7 | — |

TABLE 11
Calculated pharmacokinetic parameters following 5 mg/kg
IV dosing of Compound 169 in Male Cynomolgus Monkeys

| | 5 mg/kg IV | | | | |
|---|---|---|---|---|---|
| | C₀ | T_{1/2} | Vdss | Cl | AUCₗₐₛₜ |
| Values | ng/ml | (h) | (L/kg) | (ml/min/kg) | (h*ng/ml) |
| Mean | 10287 | 44.3 | 32.6 | 17.0 | 4679 |
| SD | 3684 | 34.9 | 21.1 | 3.24 | 1281 |

TABLE 12
Calculated pharmacokinetic parameters following 10 mg/kg
PO dosing of Compound 169 in Male Cynomolgus Monkeys

| | 10 mg/kg PO | | |
|---|---|---|---|
| | Cₘₐₓ | AUCₗₐₛₜ | |
| Values | (ng/ml) | (h*ng/ml) | % F |
| Mean | 101 | 1019 | 10.8 |
| SD | 61.4 | 470 | |

### Assay 6. Pharmacodynamics (PD) in Male Cynomolgus Monkeys

For PBMC isolation, approximately 1 mL of blood sample was collected in a BD Vacutainer® EDTA Tubes (Catalog no. 36643, Becton, Dickinson and Co., Franklin Lakes, NJ, USA) with K2-EDTA and stored at room temperature (RT). The blood was diluted 1:1 with PBS at room temperature. The tube contents were mixed gently by pipetting up and down 5 times. The PBS/blood mixture was layered carefully on the top of 2 ml Ficoll® Paque Plus (Catalog no. 17-1440-03, Cytiva, Marlborough, MA, USA) in 15 mL falcon tubes, then centrifuged at RT for 30 minutes at 600 g with acceleration set at 6 and brake set at 0. The middle ‘cloudy’ interface between plasma and Ficoll® Paque Plus containing the PBMCs was transferred to a 15 mL tube and washed once with 10 mL of PBS, then centrifuged for 10 minutes at RT at 250 g. The supernatant was discarded without disturbing the cell pellet. If red blood cells were still present, 1 mL of lysis buffer was added, incubated for 2 minutes at RT, then 10 mL PBS was added to terminate lysis, and then centrifuged for 5 minutes at RT at 250 g. The supernatant was discarded without disturbing the cell pellet and stored at −80° C. until analysis.

For cell counting, the cell suspension was diluted 1:1 with Trypan Blue dye and mixed well. The cell numbers (live and dead) were counted, and cell viability calculated. The cell numbers and cell viability of each PBMC sample were recorded.

For sample preparation for MSD analysis, 2 million cells were subjected to lysis by 100 μL of RIPA lysis and extraction buffer (Catalog no 89900, ThermoScientific, Waltham, MA, USA) with phosphatase and protease inhibitors were added for 30 minutes at 4° C. To prepare RIPA lysis and extraction buffer with phosphatase and protease inhibitors, 1 tablet of cOmplete™, 1 tablet of PhosSTOP™, 100 μL each of Phosphatase Inhibitor Cocktail 2 and 3 (all catalog no. 11836153001, 4906845001, P5726 and P0044 respectively, Sigma Aldrich, St. Louis, MO, USA) were mixed with 10 mL RIPA buffer. The lysed PBMC samples were stored at −80° C. until analysis. Samples were completely thawed before being subjected to MSD analysis. The data was normalized using protein concentration and PBMC cell numbers, separately, for comparison.

For biomarker detection of IRAK4 in samples, MSD® or Meso Scale Discovery® biomarker assays technology was utilized, which is an ELISA-based method.

For the capture or coating step, 100 μL of IRAK4 Monoclonal Antibody (2H9) (Fisher, Catalog no. MA5-15883) was added to 96 well MSD multi-array 96 well plate (MSD Catalog no. L15XA-3, MSD multi-array 96 well plate). The plate was sealed and incubated overnight at 2-8° C. The plate was washed 5×300 μL/well with tris buffered saline wash buffer. 300 μL of blocking Buffer (3% BSA in 0.1% PBST) (Wuxi, Catalog no. BB-20211112-YJC) was added. The plate was sealed and incubated for 2 h±10 minutes at room temperature without shaking. The plate was washed 5×300 μL/well with tris buffered saline wash buffer. 100 μL of standard and samples were added into each well. The plate was sealed and incubated for 1 h 10 minutes at room temperature with shaking at 500 rpm. The plate was washed 5×300 μL/well with tris buffered saline wash buffer. 100 μL of detection solution containing IRAK4 antibody (Catalog no. 4363, Cell Signaling Technology, Danvers, MA, USA) was added into each well. The plate was sealed and incubated for 1 h 10 min at RT with shaking at 500 RPM. The plate was washed 5×300 μL/well with tris buffered saline wash buffer. 100 μL of sulfotag solution containing Sulfo-Tag labeled anti-rabbit antibody (Goat) (Catalog no. R32AB-1, Meso Scale Diagnostics, Rockville, MD, USA) was added into each well. The plate was sealed and incubated for 1 h+10 minutes at room temperature with shaking at 500 RPM. The plate was washed 5×300 L/well with tris buffered saline wash buffer. 150 μL of MSD Read Buffer (Meso Scale Diagnostics, Rockville, MD, USA) was added into each well before reading.

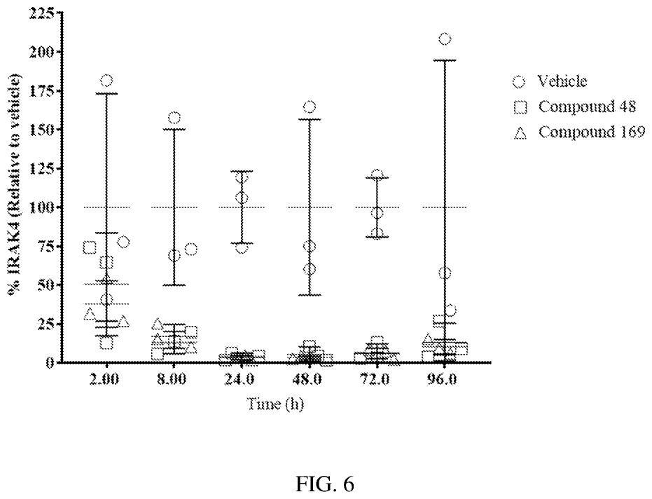

The pharmacodynamics analyses results for IRAK4 quantitation are shown in Table 13 below and in FIG. 6.

TABLE 13

| | |
|---|---|
| Time | IRAK4 change from vehicle |

| h | Compound 48 | Compound 169 |
|---|---|---|
| 2 | −50% | −62% |
| 8 | −87% | −83% |
| 24 | −96% | −97% |
| 48 | −94% | −97% |
| 72 | −94% | −94% |
| 96 | −87% | −90% |

What is claimed is:

1. A compound of Formula (A):
IRAK-L-DSM (A),
or a pharmaceutically acceptable salt thereof, wherein:
DSM is a degradation signaling moiety that is covalently attached to the linker L;
L is a linker that covalently attaches IRAK to DSM; and
IRAK is an IRAK4 binding moiety represented by Formula (I) that is covalently attached to linker L; 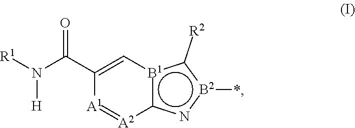
wherein:
A¹ is selected from N, CH and CR³, and A² is selected from N, CH and CR⁴, provided only one of A¹ or A² may be N;
one of B¹ and B² is N, and the other is C;
R¹ is selected from:
i. phenyl optionally substituted with 1 to 3 R⁵,
ii. a 5 or 6 membered heteroaryl having 1 to 3 heteroatoms independently selected from nitrogen, oxygen and sulfur, said heteroaryl is optionally substituted with 1 to 3 R⁵,
iii. a 5 or 6 membered partially or fully saturated heterocycle having 1 to 2 heteroatoms independently selected from oxygen and nitrogen, said heterocycle may be optionally substituted with 1 to 3 R⁵,
iv. a partially or fully saturated C₃₋₆ cycloalkyl which may be optionally substituted with 1 to 3 R⁵,
v. a 7 to 10 membered fused heterobicyclic ring system having 1, 2 or 3 heteroatoms independently selected from nitrogen and oxygen, said heterobicylic ring system is optionally substituted with 1 to 3 R⁵, and
vi. a 7 to 10 membered fused carbobicyclic ring system, said carbobicyclic ring system is optionally substituted with 1 to 3 R⁵;
R² is hydrogen, C₁₋₄ alkyl or halogen;
R³ and R⁴ are each independently selected from halogen, C₁₋₄alkyl, nitrile and —OR⁶, wherein the C₁₋₄alkyl is optionally substituted with C₁₋₄alkoxy or at least one halogen;
R⁵ for each occurrence, is independently selected from CN, hydroxyl, C₁₋₄ alkyl, oxo, halogen, —NR⁸R⁹, C₁₋₄ alkoxy, —O—C₁₋₄ alkyl, C₃₋₆cycloalkyl, —C₁₋₄alkyl-C₃₋₆cycloalkyl, C(O)NR¹⁰R¹¹, a C₄₋₇ heterocycle, and a 5 or 6 membered heteroaryl having 1 to 2 heteroatoms independently selected from nitrogen, oxygen and sulfur, said C₁₋₄ alkyl is optionally substituted with one or more substituents independently selected from CN, halo, C₁₋₄alkoxy, and hydroxyl, said C₃₋₆cycloalkyl and heteroaryl is optionally substituted with 1 to 2 substituents independently selected from the group consisting of C₁₋₄ alkyl, hydroxyl and halogen; or two R⁵ groups together with the intervening atoms can form a ring selected from phenyl, C₄₋₆ carbocycle, C₄₋₆ heterocycle, or a 7-membered bridged ring system optionally having 1 heteroatom selected from nitrogen and oxygen, wherein said phenyl, C₄₋₆ carbocycle and C₄₋₆ heterocycle are each optionally substituted with 1 to 2 C₁₋₄ alkyl, halogen or C₁₋₄ haloalkyl;
R⁶ is hydrogen, C₁₋₅alkyl, C₃₋₆cycloalkyl, a 4 to 7 membered partially or fully saturated heterocycle containing 1 or 2 heteroatoms selected from nitrogen and oxygen, a 5 to 10 membered spiro carbocyclic ring and a 4 to 10 membered heterocycle having 1 to 2 heteroatoms independently selected from nitrogen and oxygen; wherein the C₁₋₅alkyl represented by R⁶ is optionally substituted with 1 to 3 substituents R^{6a} independently selected from halogen, hydroxyl, C₁₋₅alkyl, C₁₋₄alkoxy, C₁₋₄ haloalkoxy, C₃₋₆cycloalkyl, phenyl, a 4 to 7 membered partially or fully saturated heterocycle containing 1 or 2 heteroatoms selected from nitrogen and oxygen, and a fully saturated 5 to 8 membered bridged-heterocyclic ring system having 1 to 2 heteroatoms independently selected from nitrogen and oxygen; wherein the C₃₋₆cycloalkyl represented by R⁶ is optionally substituted with 1 to 3 substituents R^{b} independently selected from halogen, C₁₋₄alky, C₁₋₄ haloalkyl, and C₁₋₄alkoxy; wherein the 4 to 7 membered partially or fully saturated heterocycle, the 5 to 10 membered spiro carbocyclic ring and 5 to 10 membered spiro heterobicyclic ring system represented by R⁶ is optionally substituted with 1 to 3 substituents R^{6c} independently selected from C₁₋₄alkyl and oxo; and wherein said C₃₋₆cycloalkyl, phenyl, 4 to 7 membered partially or fully saturated heterocycle represented by R^{6a} are optionally substituted with 1 to 3 R⁷;
each R⁷ is independently selected from oxo, halogen, C₁₋₄ haloalkyl and C₁₋₄ alkyl;
R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl;
R¹⁰ and R¹¹ are each independently selected from hydrogen and C₁₋₄ alkyl; and [symbol]
represents a bond to the linker L.

2. The compound of claim 1, or a pharmaceutically acceptable salt thereof, wherein IRAK is an IRAK4 binding moiety represented by Formula (IA), (IB), or (IC):

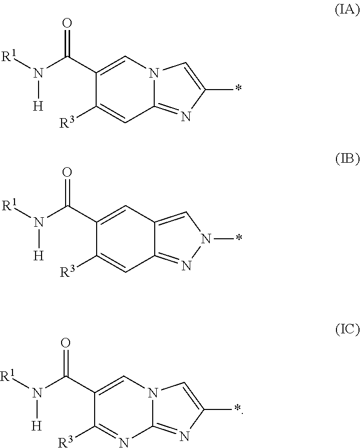

3. The compound of claim 1, or a pharmaceutically acceptable salt thereof, wherein IRAK is an IRAK4 binding moiety represented by Formula (IA) or (IB):

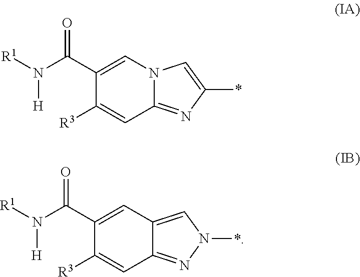

4. The compound of any one of claims 1 to 3, or a pharmaceutically acceptable salt thereof, wherein R¹ is selected from phenyl optionally substituted with 1 to 3 R⁵; 5 or 6 membered heteroaryl having 1 to 2 nitrogen atoms, said heteroaryl is optionally substituted with 1 to 3 R⁵; 5 or 6 membered partially or fully saturated heterocycle having 1 to 2 heteroatoms independently selected from oxygen and nitrogen, said heterocycle may be optionally substituted with 1 to 3 R⁵; and 9 to 10 membered bicyclic heteroaryl having 1, 2 or 3 nitrogen atoms, said ring system is optionally substituted with 1 to 3 R⁵.

5. The compound of any one of claims 1 to 3, or a pharmaceutically acceptable salt thereof, wherein R¹ is selected from phenyl optionally substituted with 1 to 2 R⁵; pyrazole optionally substituted with 1 to 2 R⁵; pyridine optionally substituted with 1 to 2 R⁵; pyridone optionally substituted with 1 to 2 R⁵; pyrimidine optionally substituted with 1 to 2 R⁵; and pyrazolo[1,5-a]pyrimidine optionally substituted with 1 to 2 R⁵.

6. The compound of any one of claims 1 to 3, or a pharmaceutically acceptable salt thereof, wherein R¹ is selected from phenyl optionally substituted with 1 to 2 R⁵; pyrazole optionally substituted with 1 to 2 R⁵; pyridine optionally substituted with 1 to 2 R⁵; pyrimidine optionally substituted with 1 to 2 R⁵; and pyrazolo[1,5-a]pyrimidine optionally substituted with 1 to 2 R⁵.

7. The compound of any one of claims 1 to 3, or a pharmaceutically acceptable salt thereof, wherein R¹ is represented by one of the following formulae: 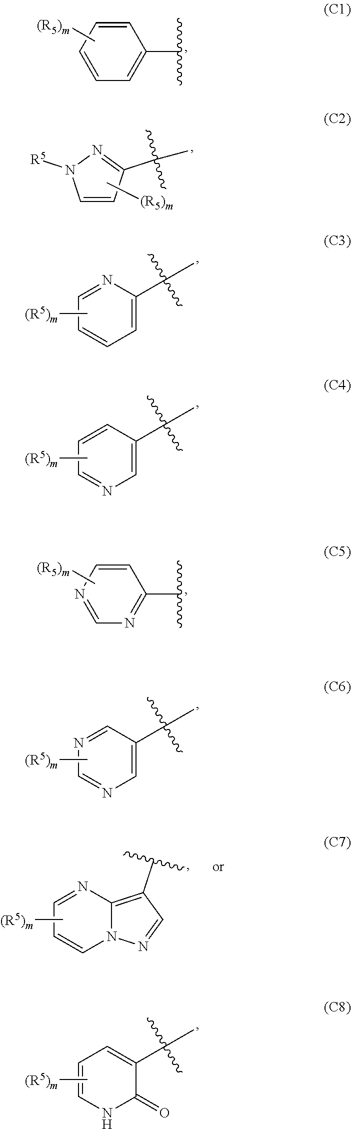
wherein m is 0, 1 or 2.

8. The compound of any one of claims 1 to 3, or a pharmaceutically acceptable salt thereof, wherein R¹ is represented by one of the following formulae: 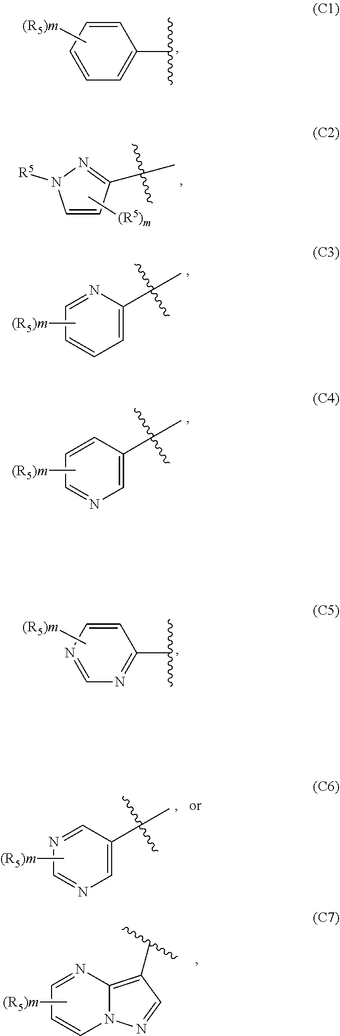
wherein m is 0, 1 or 2.

9. The compound of any one of claims 1 to 3, or a pharmaceutically acceptable salt thereof, wherein R¹ is represented by one of the following formulae:

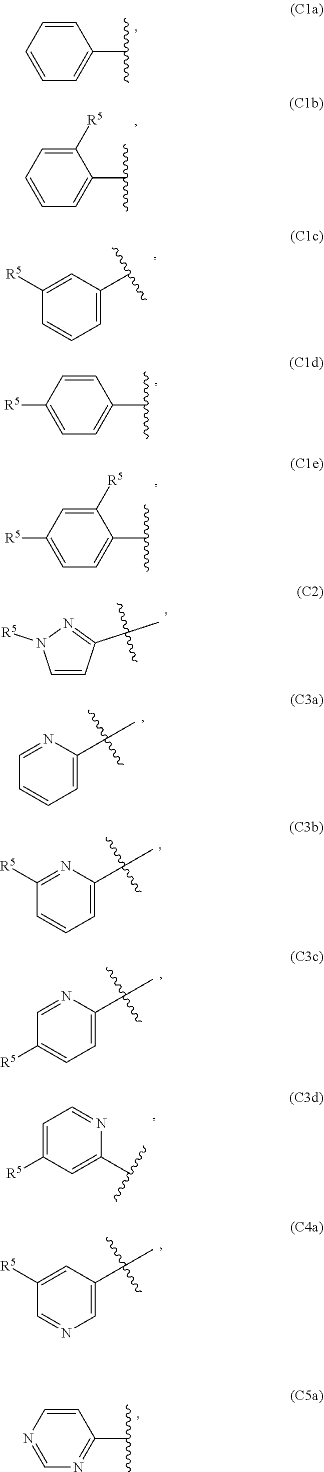

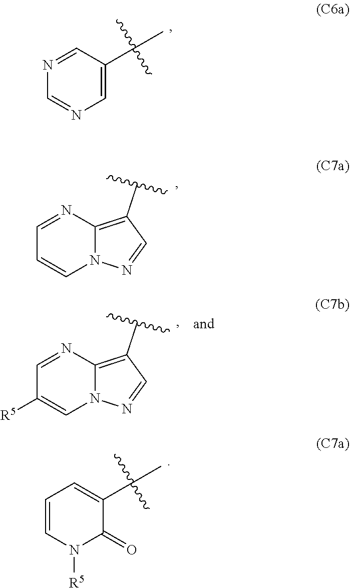

10. The compound of any one of claims 1 to 3, or a pharmaceutically acceptable salt thereof, wherein R¹ is represented by one of the following formulae:

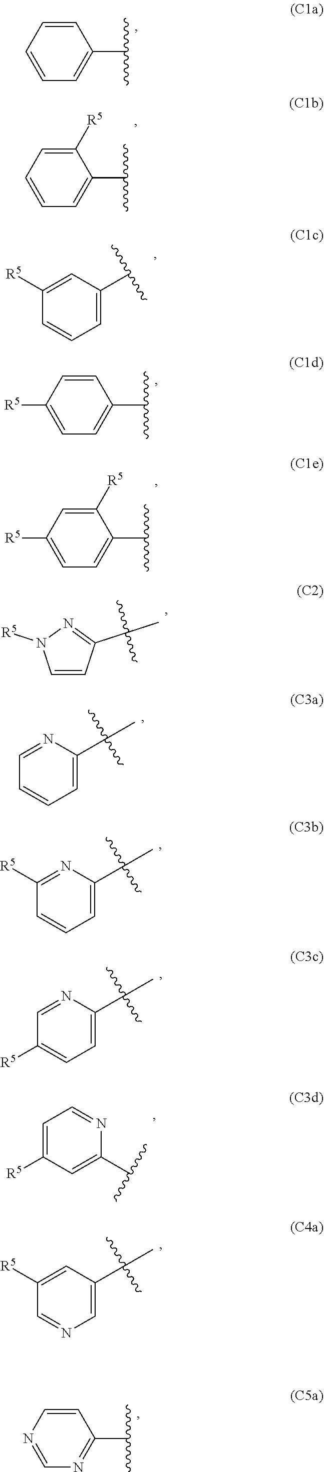

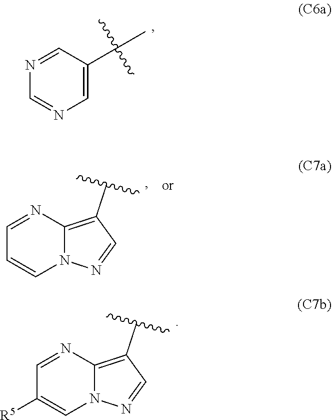

11. The compound of any one of claims 1 to 3, or a pharmaceutically acceptable salt thereof, wherein R¹ is represented by one of the following formulae:

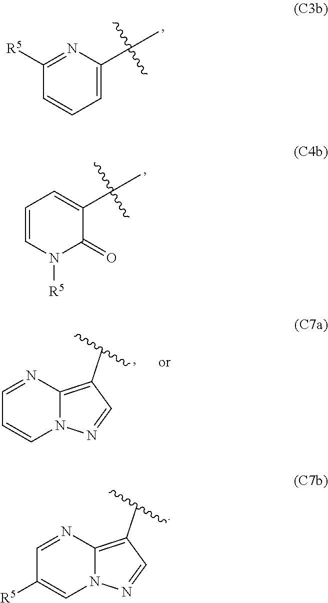

12. The compound of any one of claims 1 to 3, or a pharmaceutically acceptable salt thereof, wherein R¹ is represented by one of the following formulae:

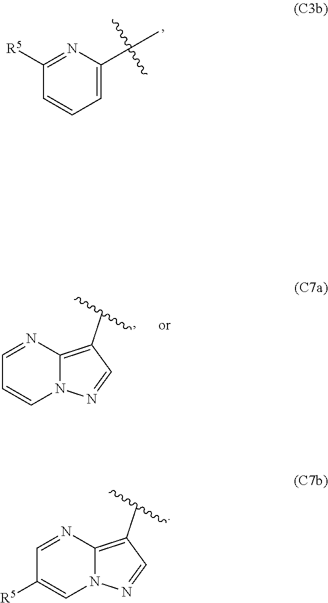

13. The compound of any one of claims 1 to 12, or a pharmaceutically acceptable salt thereof, wherein R² is hydrogen.

14. The compound of claim 1, or a pharmaceutically acceptable salt thereof, wherein IRAK is an IRAK4 binding moiety represented by one of the following formulae:

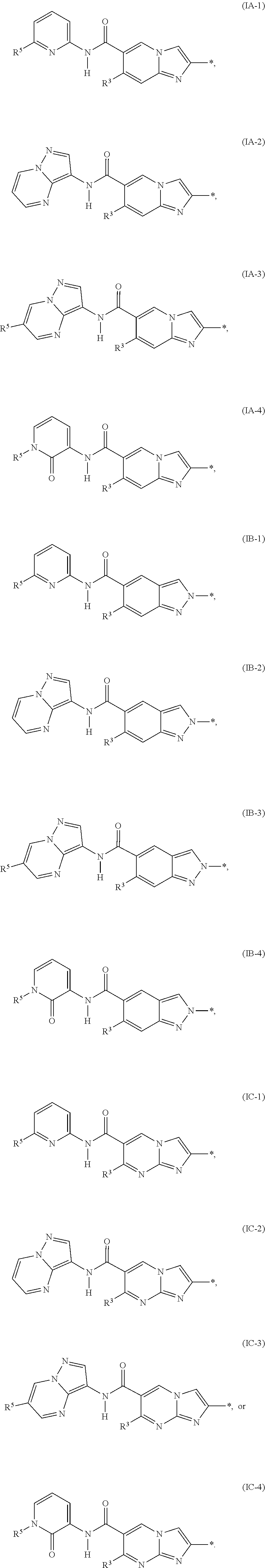

15. The compound of claim 1, or a pharmaceutically acceptable salt thereof, wherein IRAK is an IRAK4 binding moiety represented by one of the following formulae:

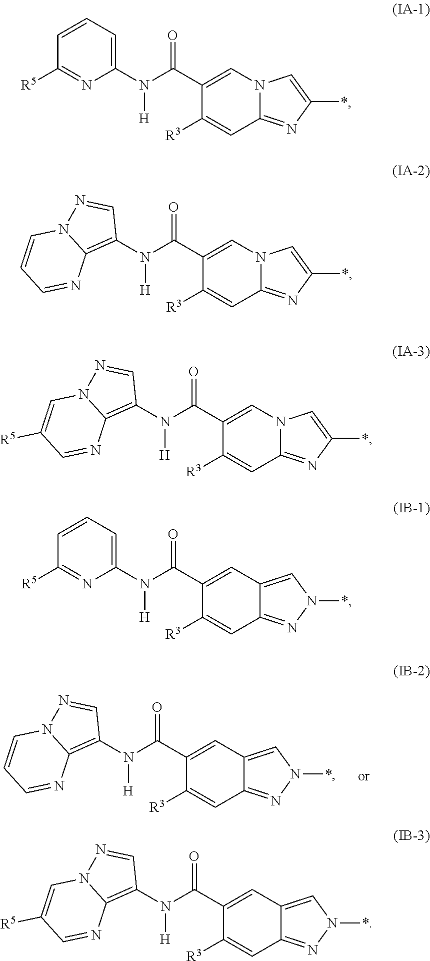

16. The compound of any one of claims 1 to 15, or a pharmaceutically acceptable salt thereof, wherein:
R³ is C₁₋₄alkyl or —OR⁶, wherein the C₁₋₄alkyl is optionally substituted with at least one halogen; and
R⁶ is C₁₋₅alkyl.

17. The compound of any one of claims 1 to 15, or a pharmaceutically acceptable salt thereof, wherein:
R³ is —CF₃ or —O—CH(CH₃)₂.

18. The compound of any one of claims 1 to 15, or a pharmaceutically acceptable salt thereof, wherein R³ is —O—CH(CH₃)₂.

19. The compound of any one of claims 1 to 18, or a pharmaceutically acceptable salt thereof, wherein R⁵ for each occurrence, is independently selected from C₁₋₄ alkyl, halogen, C₁₋₄ haloalkyl, and C₃₋₄cycloalkyl, and wherein said C₃₋₄cycloalkyl is optionally substituted with 1 halo.

20. The compound of any one of claims 1 to 18, or a pharmaceutically acceptable salt thereof, wherein R⁵ for each occurrence, is independently selected from C₁₋₄ alkyl, halogen, and C₁₋₄ haloalkyl.

21. The compound of claim 19 or 20, or a pharmaceutically acceptable salt thereof, wherein R⁵ for each occurrence, is independently selected from —CH₃, —CHF₂, —CF₃, F, cyclopropyl, and

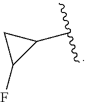 [SMILES: *C1CC1F]

22. The compound of claim 19 or 20, or a pharmaceutically acceptable salt thereof, wherein R⁵ for each occurrence, is independently selected from —CH₃, —CHF₂, —CF₃, and F.

23. The compound of claim 1, or a pharmaceutically acceptable salt thereof, wherein IRAK is an IRAK binding moiety represented by one of the following formulae: 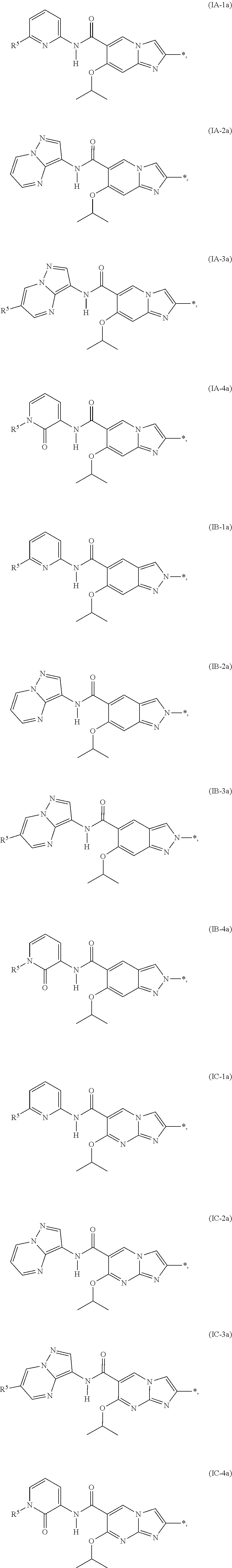
wherein R⁵ is C₁₋₃ alkyl C₁₋₃ haloalkyl, or C₃₋₄cycloalkyl, and wherein said C₃₋₄cycloalkyl is optionally substituted with 1 halo.

24. The compound of claim 1, or a pharmaceutically acceptable salt thereof, wherein IRAK is an IRAK binding moiety represented by one of the following formulae: 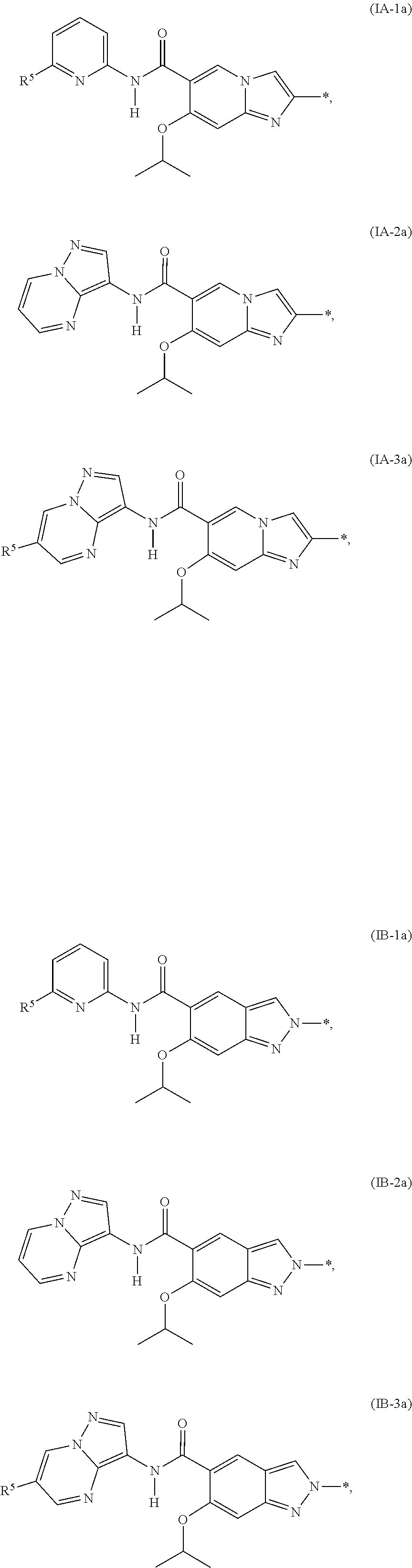
wherein R⁵ is C₁₋₃ alkyl or C₁₋₃ haloalkyl.

25. The compound of claim 23 or 24, or a pharmaceutically acceptable salt thereof, wherein R⁵ is CH₃, CHF₂, CF₃, cyclopropyl, or

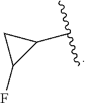 [SMILES: *C1CC1F]

26. The compound of claim 23 or 24, or a pharmaceutically acceptable salt thereof, wherein R⁵ is CH₃, CHF₂, or CF₃.

27. The compound of any one of claims 1 to 26, or a pharmaceutically acceptable salt thereof, wherein DSM is a degradation signaling moiety of formula (D): 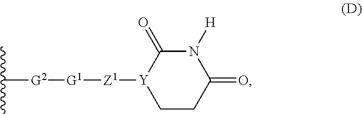
wherein: 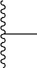
represents a bond to the linker L;
Y is CR^{D1} or N;
Z¹ is selected from a bond, —NR^{D2}—, —O— and —CH₂—;
G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more R^{D3};
G² is selected from Het₁, *—NR^{D4}—C₄₋₆ cycloalkyl-*, *—NR^{D4}-Het₁-*, *—NR^{D4}-Het₁-C₁₋₄ alkyl-*, *—C₁₋₄ alkyl-C(R^{D1})=Het₁-*, *—C(O)—C₁₋₄ alkyl-Het₁-*, *-Het₁-C₁₋₆ alkyl-*, *-Het₁-O—*, *—C(O)—C₁₋₄ alkyl-Het₁-C(O)—*, *—C(O)-Het₁-C(O)—*, *—C(O)-phenyl-C₁₋₄ alkyl-NHC(O)—*, *—C(O)—C₁₋₆ alkyl-NR^{D4}—, *—NR^{D4}-cycloalkyl-**, *—O-Het₁-*, or *—NR^{D4}—C₁₋₄alkyl-Het₁-*; wherein *- represents a bond to the linker L, and *- represents a bond to G¹;
Het₁ is 4- to 7-membered monocyclic heterocycle or 7- to 11-membered bicyclic heterocycle, each of which is optionally substituted with one or more R^{D5};
R^{D1} is selected from H, C₁₋₆ alkyl or halogen;
R^{D2} is H or C₁₋₃ alkyl;
R^{D3} is, for each occurrence, independently selected from H, halogen, C₁₋₄ alkyl and C₁₋₄haloalkyl;
R^{D4} is H or C₁₋₃ alkyl; and
R^{D5} is, for each occurrence, independently selected from H, halogen, hydroxyl, C₁₋₄ alkyl, C₁₋₄haloalkyl and C₁₋₄ alkoxy.

28. The compound of any one of claims 1 to 26, or a pharmaceutically acceptable salt thereof, wherein DSM is a degradation signaling moiety of formula (D): 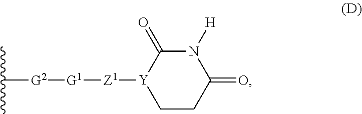
wherein: 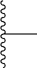
represents a bond to the linker L;
Y is CR^{D1} or N;
Z¹ is selected from a bond, —NR^{D2}—, —O— and —CH₂—;
G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more R^{D3};
G² is selected from Het₁, *—NR^{D4}-Het₁-*, *—NR^{D4}-Het₁-C₁₋₄ alkyl-*, *—C₁₋₄ alkyl-C(R^{D1})=Het₁-*, *—C(O)—C₁₋₄ alkyl-Het₁-*, *-Het₁-C₁₋₆ alkyl-*, *-Het₁-O—*, *—C(O)—C₁₋₄ alkyl-Het₁-C(O)—*, *—C(O)-Het₁-C(O)—*, *—C(O)-phenyl-C₁₋₄ alkyl-NHC(O)—*, wherein *-represents a bond to the linker L, and *- represents a bond to G¹;
Het₁ is 4- to 7-membered monocyclic heterocycle or 7- to 11-membered bicyclic heterocycle, each of which is optionally substituted with one or more R^{D5};
R^{D1} is selected from H, C₁₋₆ alkyl or halogen;
R^{D2} is H or C₁₋₃ alkyl;
R^{D3} is, for each occurrence, independently selected from H, halogen, C₁₋₄ alkyl and C₁₋₄haloalkyl;
R^{D4} is H or C₁₋₃ alkyl; and
R^{D5} is, for each occurrence, independently selected from H, halogen, hydroxyl, C₁₋₄ alkyl, C₁₋₄haloalkyl and C₁₋₄ alkoxy.

29. The compound of claim 27 or 28, or a pharmaceutically acceptable salt thereof, wherein Het₁ is a 4 to 7 membered monocyclic saturated heterocycle containing 1 or 2 nitrogen atoms or a 7 to 8 membered saturated spiro bicyclic heterocycle containing 1 or 2 nitrogen atoms, each of which is optionally substituted with 1 or 2 R^{D5}.

30. The compound of claim 27 or 28, or a pharmaceutically acceptable salt thereof, wherein Het₁ is piperidine, piperazine, 1,4-diazepane, morpholine, 2-azaspiro[3.3]heptane, 2,5-diazaspiro[3.4]octane, 2,7-diazaspiro[3.5]nonane, or 2,6-diazaspiro[3.3]heptane, each of which is optionally substituted with 1 or 2 R^{D5}.

31. The compound of claim 27 or 28, or a pharmaceutically acceptable salt thereof, wherein Het₁ is piperidine, piperazine, 2-azaspiro[3.3]heptane, or 2,6-diazaspiro[3.3]heptane, each of which is optionally substituted with 1 or 2 R^{D5}.

32. The compound of claim 30, or a pharmaceutically acceptable salt thereof, wherein Het₁ is represented by any one of the following formulae: 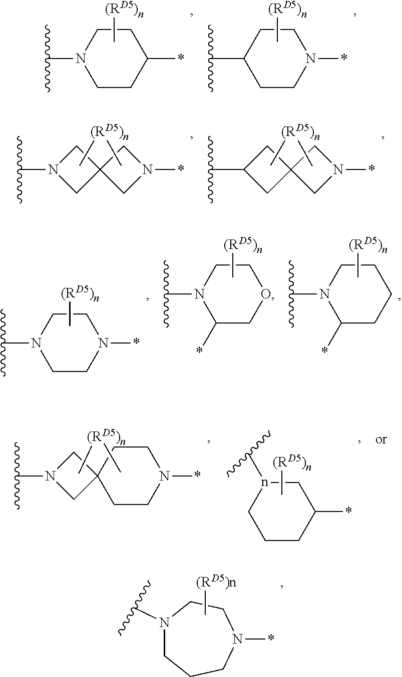
wherein n is 0, 1 or 2, 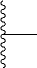
represents a bond directly or indirectly to the linker L, and -* represents directly or indirectly to G¹.

33. The compound of any one of claims 1 to 32, or a pharmaceutically acceptable salt thereof, wherein DSM is a degradation signaling moiety of formula (D-I), (D-II), (D-III) (D-IV) or (D-V): 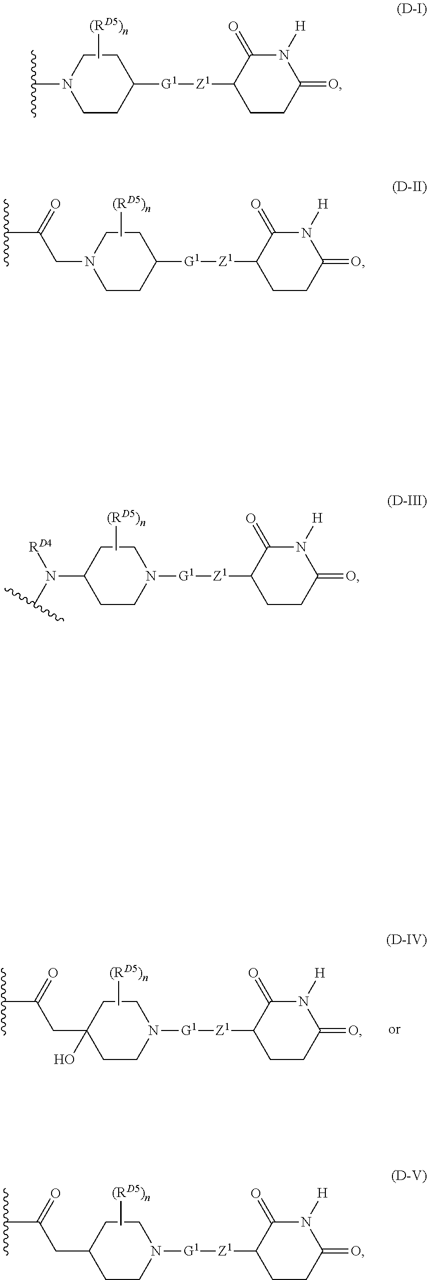
wherein: 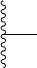
represents a bond to the linker L;
Z¹ is selected from a bond, —NR^{D2}— and —O—;
G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more R^{D3};
R^{D2} is C₁₋₃ alkyl;
R^{D3} is, for each occurrence, independently selected from H, halogen and C₁₋₄ alkyl;
R^{D4} is H or C₁₋₃ alkyl;
R^{D5} is halogen; and
n is 0, 1 or 2.

34. The compound of any one of claims 1 to 32, or a pharmaceutically acceptable salt thereof, wherein DSM is a degradation signaling moiety of formula (D-I), (D-II), (D-III) (D-IV) or (D-V): 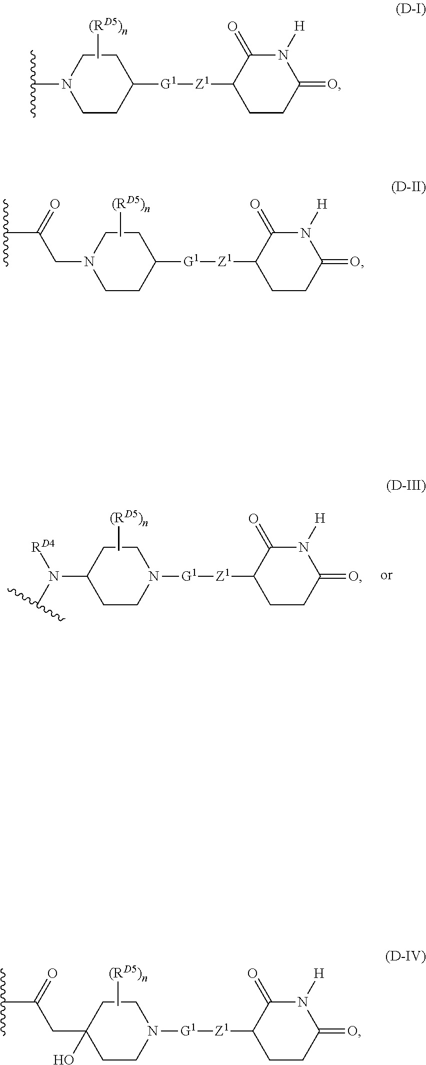
wherein 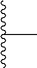
represents a bond to the linker L;
Z¹ is selected from a bond, —NR^{D2}— and —O—;
G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more R^{D3};
R^{D2} is C₁₋₃ alkyl;
R^{D3} is, for each occurrence, independently selected from H, halogen and C₁₋₄ alkyl;
R^{D4} is C₁₋₃ alkyl;
R^{D5} is halogen; and
n is 0, 1 or 2.

35. The compound of any one of claims 27 to 34, or a pharmaceutically acceptable salt thereof, wherein G¹ is selected from phenyl, pyrazolyl, pyridinyl, pyrimidinyl, 1,3-dihydro-2H-benzo[d]imidazol-2-one, benzo[d]oxazol-2(3H)-one, 7,9-dihydro-8H-purin-8-one, 1,3-dihydro-2H-imidazo[4,5-b]pyridin-2-one, pyrazinyl, indazolyl, and indolyl, each of which is optionally substituted with 1 or 2 R^{D3}.

36. The compound of any one of claims 27 to 34, or a pharmaceutically acceptable salt thereof, wherein G¹ is selected from phenyl, pyrazolyl, pyridinyl and pyrimidinyl, 1,3-dihydro-2H-benzo[d]imidazol-2-one, pyrazolo[1,5-a]pyridinyl, imidazo[1,2-a]pyridinyl, indazolyl, and indolyl, each of which is optionally substituted with 1 or 2 R^{D3}.

37. The compound of any one of claims 27 to 34, or a pharmaceutically acceptable salt thereof, wherein G¹ is represented by any one of the following formulae: 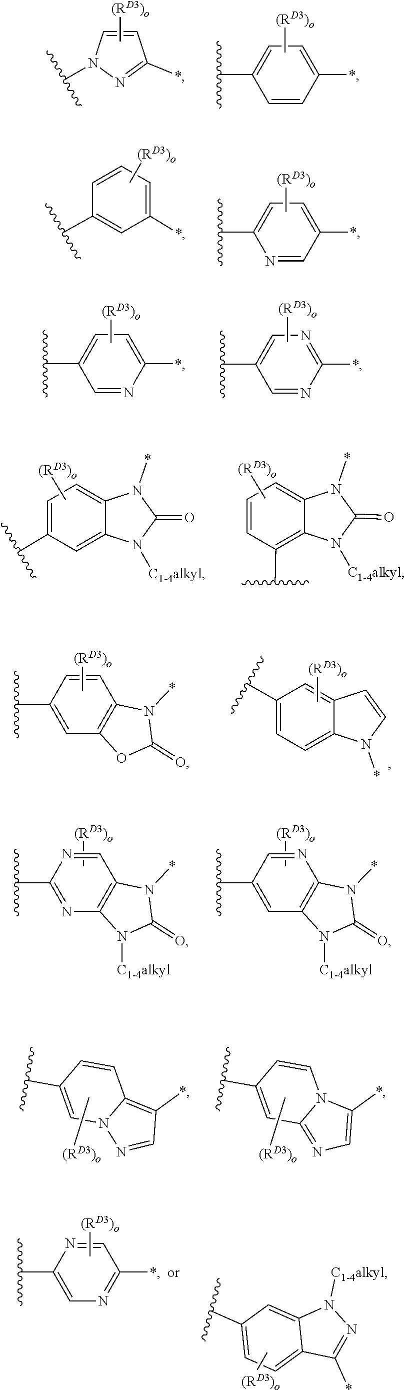
wherein o is 0, 1 or 2, 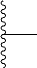
represents a bond to G², and -* represents a bond to Z¹.

38. The compound of any one of claims 27 to 34, or a pharmaceutically acceptable salt thereof, wherein G¹ is 6- to 10-membered aryl or 5- to 10-membered heteroaryl; wherein the 6- to 10-membered aryl and 5- to 10-membered heteroaryl represented by G¹ are each optionally substituted with 1 or 2 R^{D3}.

39. The compound of any one of claims 27 to 34, or a pharmaceutically acceptable salt thereof, wherein G¹ is represented by any one of the following formulae: 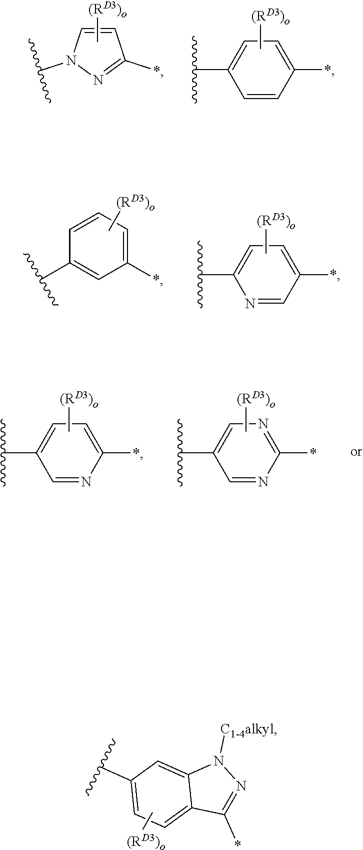
wherein o is 0, 1 or 2, 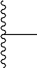
represents a bond to G², and -* represents a bond to Z¹.

40. The compound of any one of claims 1 to 39, or a pharmaceutically acceptable salt thereof, wherein R^{D1} is H, —CH₃ or F.

41. The compound of any one of claims 1 to 40, or a pharmaceutically acceptable salt thereof, wherein R^{D2} is H.

42. The compound of any one of claims 1 to 41, or a pharmaceutically acceptable salt thereof, wherein R^{D3} is, for each occurrence, independently selected from H, Cl, F and —CH₃.

43. The compound of any one of claims 1 to 42, or a pharmaceutically acceptable salt thereof, wherein R^{D4} is —CH₃.

44. The compound of any one of claims 1 to 43, or a pharmaceutically acceptable salt thereof, wherein R^{D5} for each occurrence, is independently F or OH.

45. The compound of any one of claims 1 to 26, or a pharmaceutically acceptable salt thereof, wherein DSM represents any one of the following attached to L:

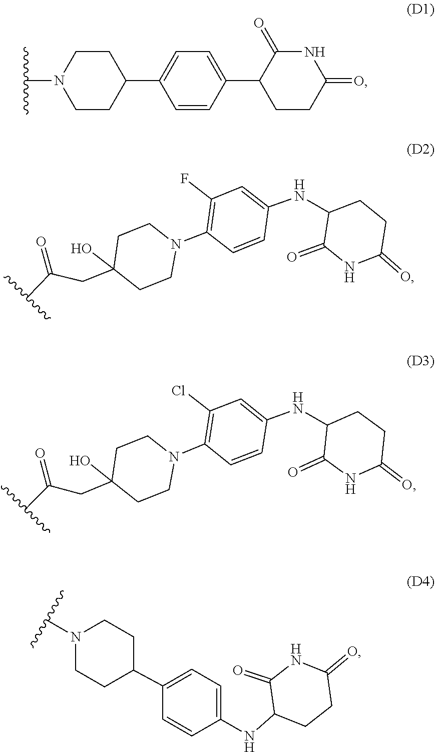 [SMILES: *N1CCC(c2ccc(C3CCC(=O)NC3=O)cc2)CC1 ; *C(=O)CC1(O)CCN(c2ccc(NC3CCC(=O)NC3=O)cc2F)CC1 ; *C(=O)CC1(O)CCN(c2ccc(NC3CCC(=O)NC3=O)cc2Cl)CC1 ; *N1CCC(c2ccc(NC3CCC(=O)NC3=O)cc2)CC1]

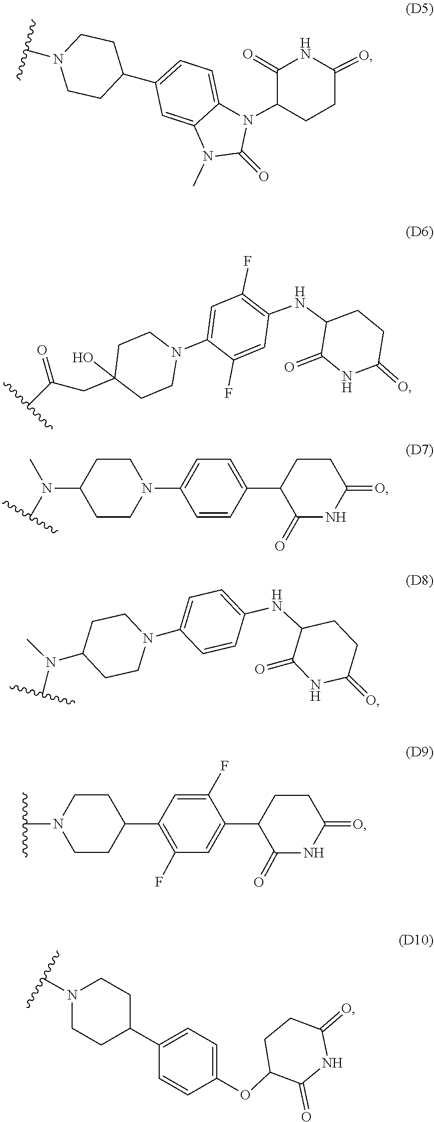 [SMILES: *N1CCC(c2ccc3c(c2)n(C)c(=O)n3C2CCC(=O)NC2=O)CC1 ; *C(=O)CC1(O)CCN(c2cc(F)c(NC3CCC(=O)NC3=O)cc2F)CC1 ; *N(C)C1CCN(c2ccc(C3CCC(=O)NC3=O)cc2)CC1 ; *N(C)C1CCN(c2ccc(NC3CCC(=O)NC3=O)cc2)CC1 ; *N1CCC(c2cc(F)c(C3CCC(=O)NC3=O)cc2F)CC1 ; *N1CCC(c2ccc(OC3CCC(=O)NC3=O)cc2)CC1]

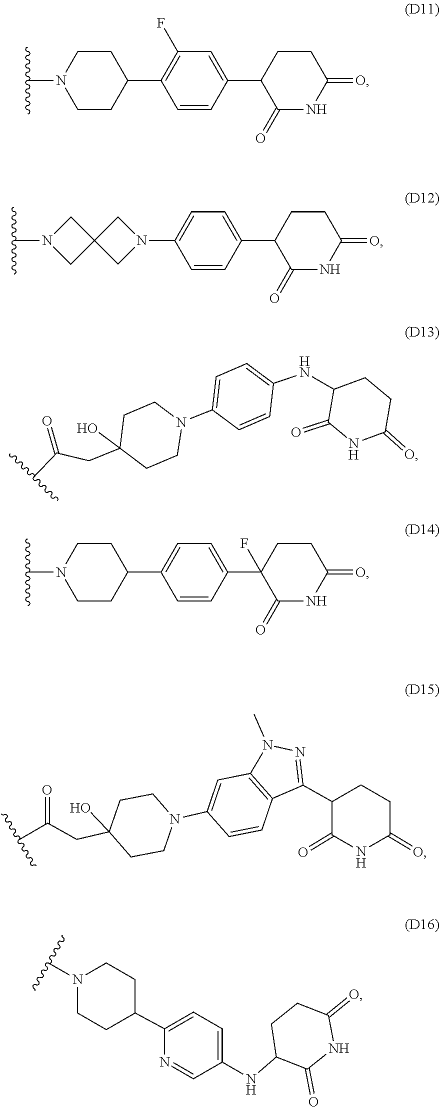 [SMILES: *N1CCC(c2ccc(C3CCC(=O)NC3=O)cc2F)CC1 ; *N1CC2(C1)CN(c1ccc(C3CCC(=O)NC3=O)cc1)C2 ; *C(=O)CC1(O)CCN(c2ccc(NC3CCC(=O)NC3=O)cc2)CC1 ; *N1CCC(c2ccc(C3(F)CCC(=O)NC3=O)cc2)CC1 ; *C(=O)CC1(O)CCN(c2ccc3c(C4CCC(=O)NC4=O)nn(C)c3c2)CC1 ; *N1CCC(c2ccc(NC3CCC(=O)NC3=O)cn2)CC1]

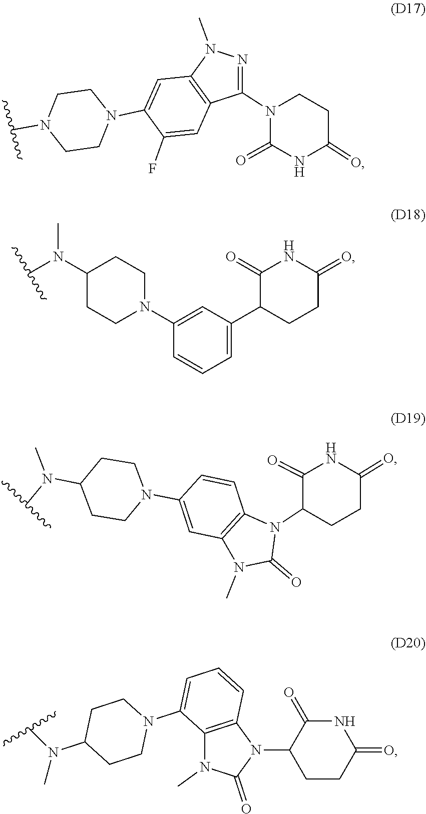 [SMILES: *N1CCN(c2cc3c(cc2F)c(N2CCC(=O)NC2=O)nn3C)CC1 ; *N(C)C1CCN(c2cccc(C3CCC(=O)NC3=O)c2)CC1 ; *N(C)C1CCN(c2ccc3c(c2)n(C)c(=O)n3C2CCC(=O)NC2=O)CC1 ; *N(C)C1CCN(c2cccc3c2n(C)c(=O)n3C2CCC(=O)NC2=O)CC1]

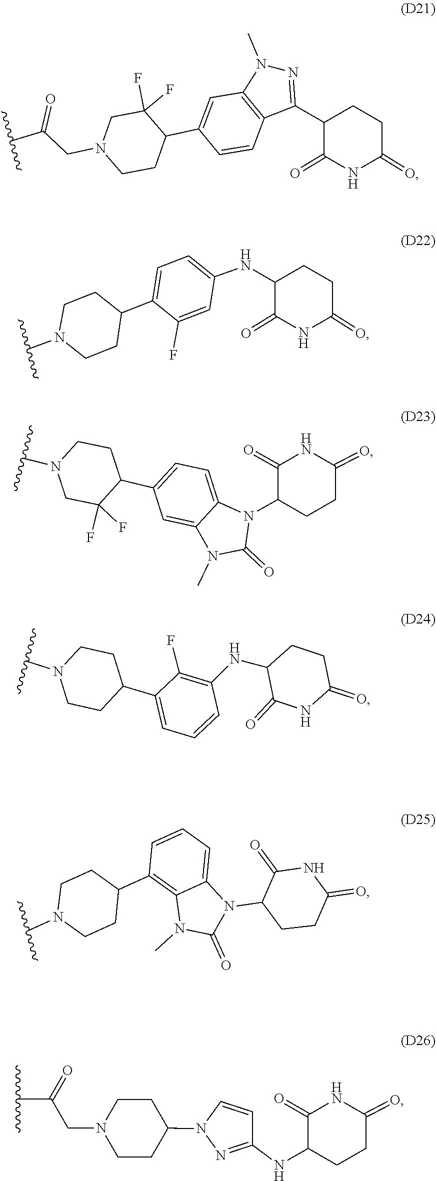 [SMILES: *C(=O)CN1CCC(c2ccc3c(C4CCC(=O)NC4=O)nn(C)c3c2)C(F)(F)C1 ; *N1CCC(c2ccc(NC3CCC(=O)NC3=O)cc2F)CC1 ; *N1CCC(c2ccc3c(c2)n(C)c(=O)n3C2CCC(=O)NC2=O)C(F)(F)C1 ; *N1CCC(c2cccc(NC3CCC(=O)NC3=O)c2F)CC1 ; *N1CCC(c2cccc3c2n(C)c(=O)n3C2CCC(=O)NC2=O)CC1 ; *C(=O)CN1CCC(n2ccc(NC3CCC(=O)NC3=O)n2)CC1]

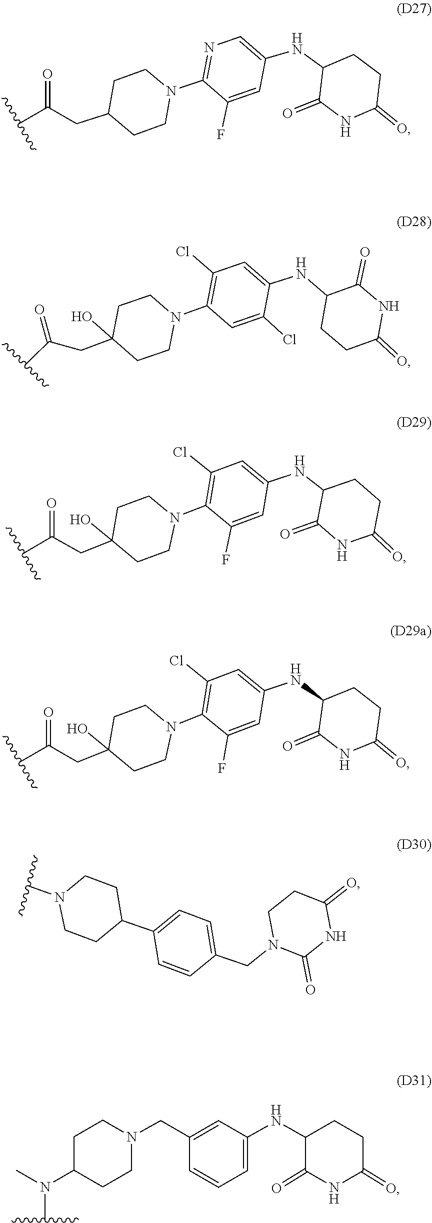 [SMILES: *C(=O)CC1CCN(c2ncc(NC3CCC(=O)NC3=O)cc2F)CC1 ; *C(=O)CC1(O)CCN(c2cc(Cl)c(NC3CCC(=O)NC3=O)cc2Cl)CC1 ; *C(=O)CC1(O)CCN(c2c(F)cc(NC3CCC(=O)NC3=O)cc2Cl)CC1 ; *C(=O)CC1(O)CCN(c2c(F)cc(N[C@H]3CCC(=O)NC3=O)cc2Cl)CC1 ; *N1CCC(c2ccc(CN3CCC(=O)NC3=O)cc2)CC1 ; *N(C)C1CCN(Cc2cccc(NC3CCC(=O)NC3=O)c2)CC1]

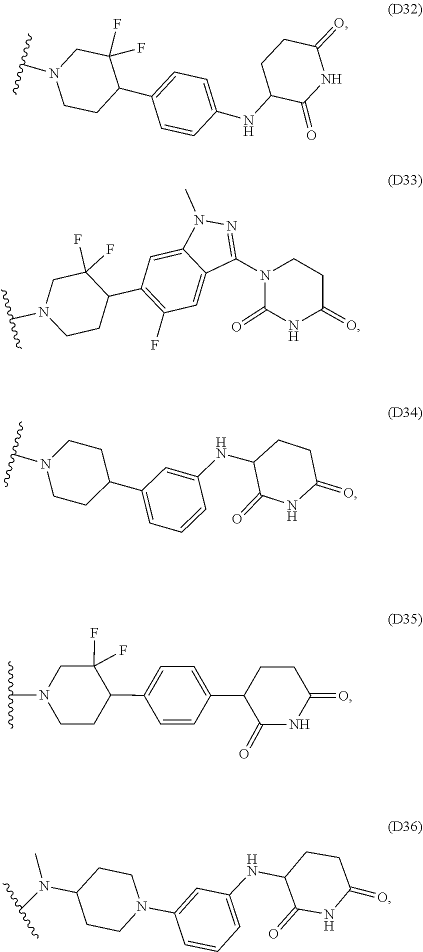 [SMILES: *N1CCC(c2ccc(NC3CCC(=O)NC3=O)cc2)C(F)(F)C1 ; *N1CCC(c2cc3c(cc2F)c(N2CCC(=O)NC2=O)nn3C)C(F)(F)C1 ; *N1CCC(c2cccc(NC3CCC(=O)NC3=O)c2)CC1 ; *N1CCC(c2ccc(C3CCC(=O)NC3=O)cc2)C(F)(F)C1 ; *N(C)C1CCN(c2cccc(NC3CCC(=O)NC3=O)c2)CC1]

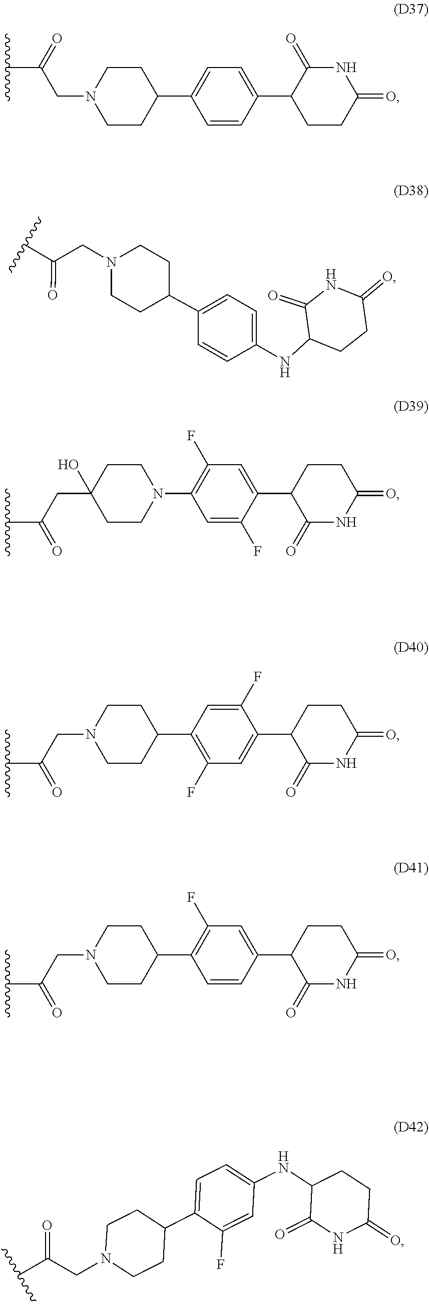 [SMILES: *C(=O)CN1CCC(c2ccc(C3CCC(=O)NC3=O)cc2)CC1 ; *C(=O)CN1CCC(c2ccc(NC3CCC(=O)NC3=O)cc2)CC1 ; *C(=O)CC1(O)CCN(c2cc(F)c(C3CCC(=O)NC3=O)cc2F)CC1 ; *C(=O)CN1CCC(c2cc(F)c(C3CCC(=O)NC3=O)cc2F)CC1 ; *C(=O)CN1CCC(c2ccc(C3CCC(=O)NC3=O)cc2F)CC1 ; *C(=O)CN1CCC(c2ccc(NC3CCC(=O)NC3=O)cc2F)CC1]

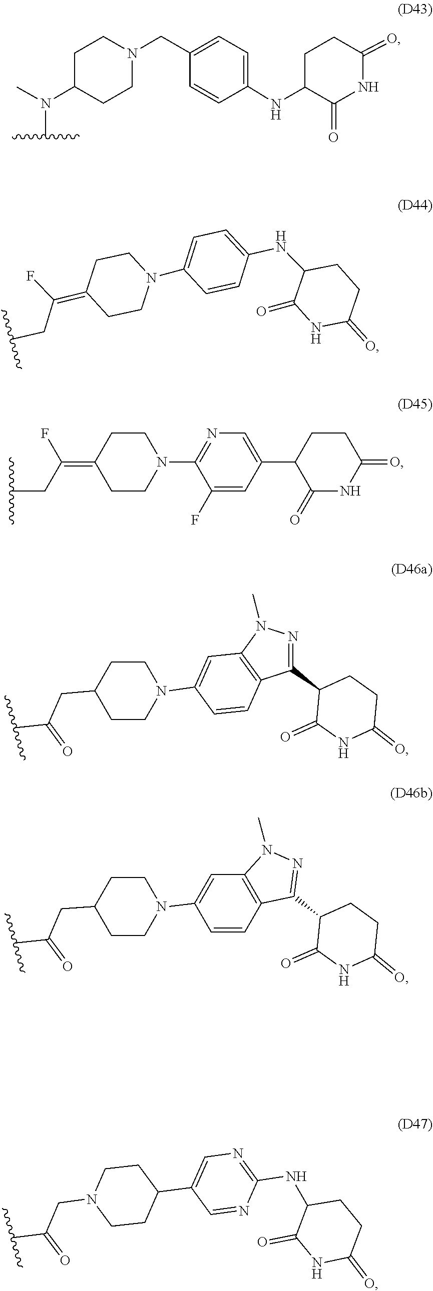 [SMILES: *N(C)C1CCN(Cc2ccc(NC3CCC(=O)NC3=O)cc2)CC1 ; *CC(F)=C1CCN(c2ccc(NC3CCC(=O)NC3=O)cc2)CC1 ; *CC(F)=C1CCN(c2ncc(C3CCC(=O)NC3=O)cc2F)CC1 ; *C(=O)CC1CCN(c2ccc3c([C@H]4CCC(=O)NC4=O)nn(C)c3c2)CC1 ; *C(=O)CC1CCN(c2ccc3c([C@@H]4CCC(=O)NC4=O)nn(C)c3c2)CC1 ; *C(=O)CN1CCC(c2cnc(NC3CCC(=O)NC3=O)nc2)CC1]

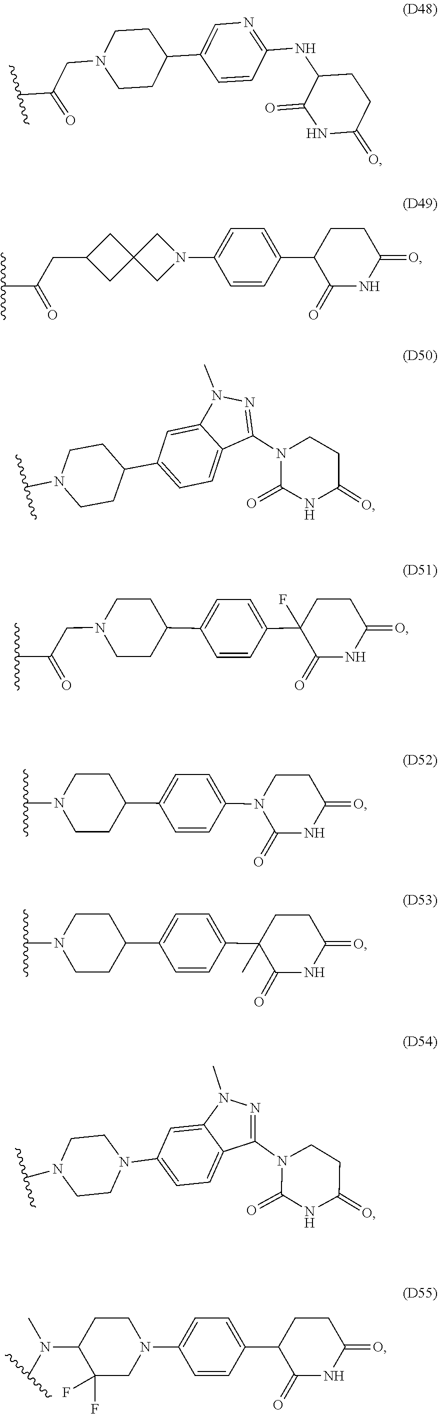

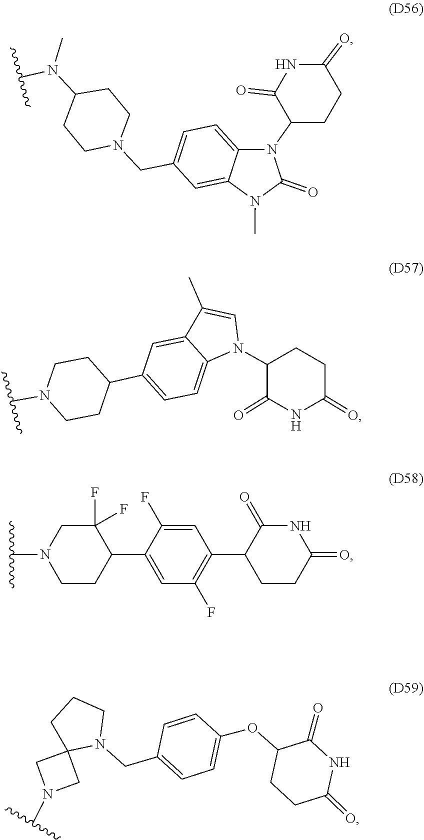 [SMILES: *N(C)C1CCN(Cc2ccc3c(c2)n(C)c(=O)n3C2CCC(=O)NC2=O)CC1 ; *N1CCC(c2ccc3c(c2)c(C)cn3C2CCC(=O)NC2=O)CC1 ; *N1CCC(c2cc(F)c(C3CCC(=O)NC3=O)cc2F)C(F)(F)C1 ; *N1CC2(CCCN2Cc2ccc(OC3CCC(=O)NC3=O)cc2)C1]

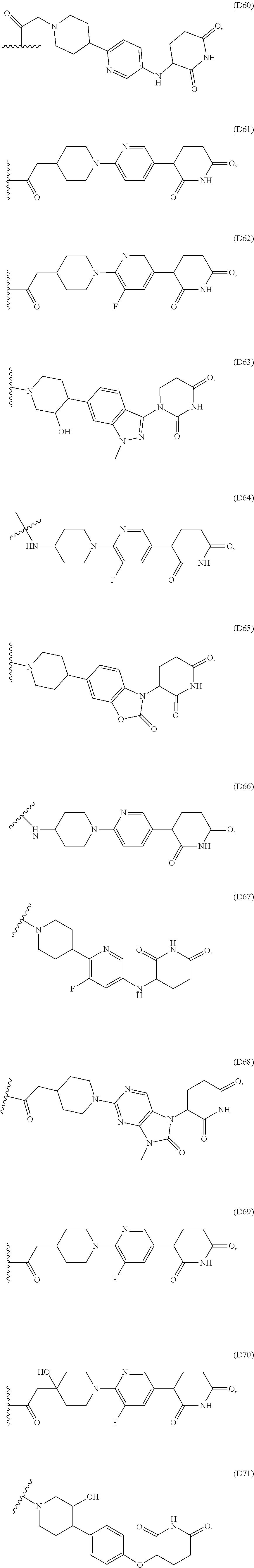

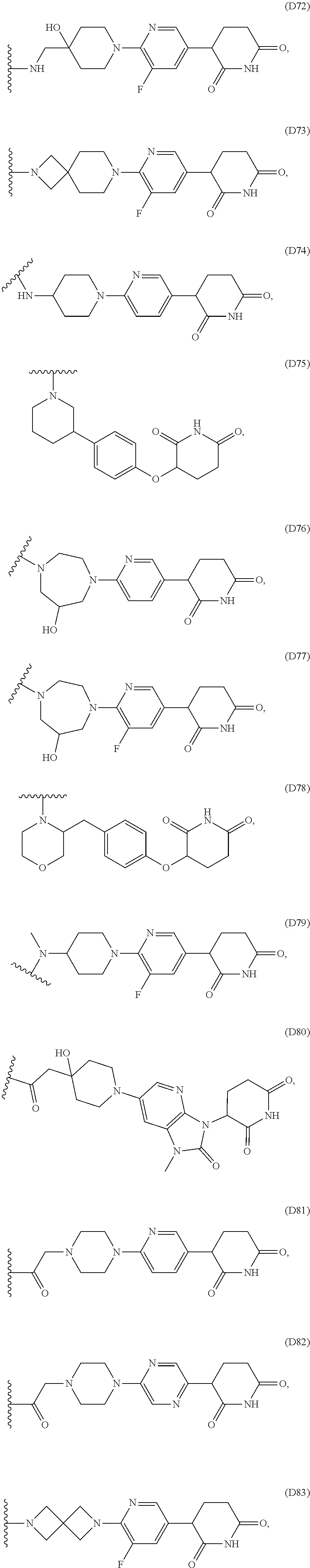

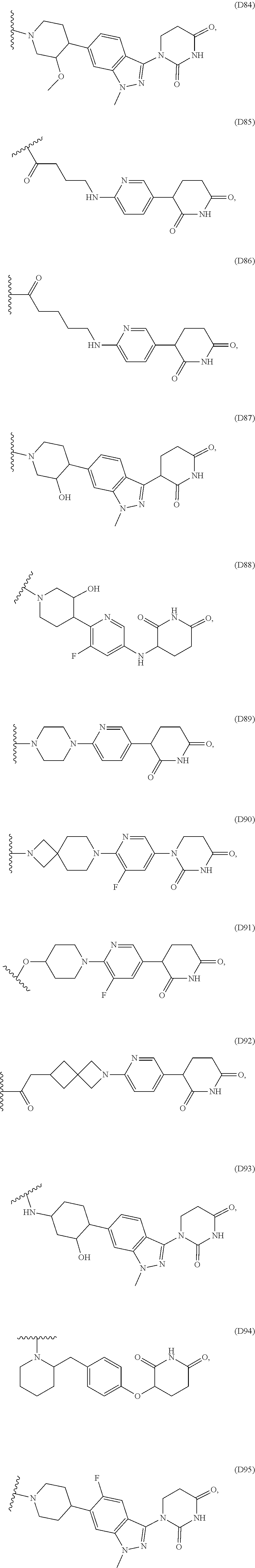

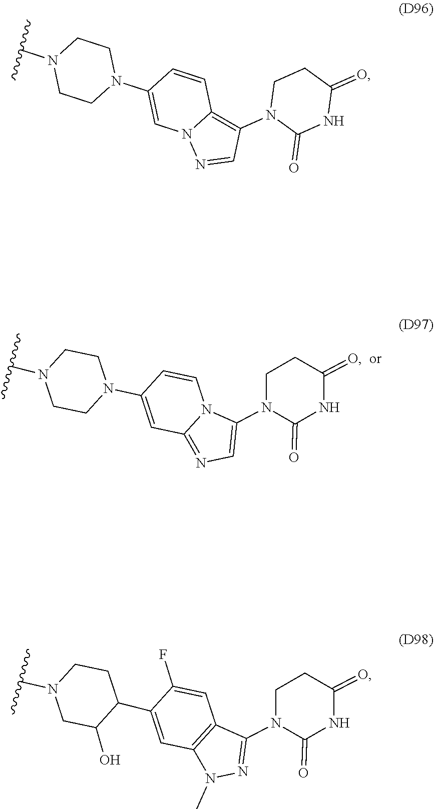

46. The compound of any one of claims 1 to 45, or a pharmaceutically acceptable salt thereof, wherein L is a bond, C₁₋₈ alkyl or is represented by formula (L-1), (L-2) or (L-3): 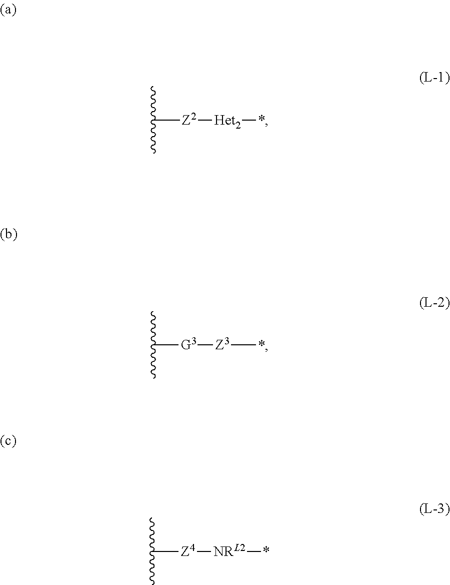
wherein:
Z² is a bond or C₁₋₄ alkyl optionally substituted with one or more halogen;
Het₂ is 4- to 7-membered heterocycle optionally substituted by one or more R^{L1};
G³ is C₃₋₇ cycloalkyl or 4- to 7-membered heterocycle; wherein the C₃₋₇ cycloalkyl and 4- to 7-membered heterocycle represented by G³ are each optionally substituted with one or more R^{L3};
Z³ is C₁₋₄ alkyl, —C(O)—, or *—C₁₋₄ alkyl-C(O)—*, wherein *- represents a bond connected to G³; -* is a bond connected to the DSM; and the C₁₋₄ alkyl is optionally substituted with one or more halogen;
Z⁴ is C₁₋₄ alkyl optionally substituted by R^{L4};
R^{L1} is, for each occurrence, independently selected from H, halogen, C₁₋₄ alkyl and C₁₋₄haloalkyl;
R^{L2} is H or C₁₋₄ alkyl;
R^{L3} is, for each occurrence, independently selected from H, halogen, C₁₋₄ alkyl and C₁₋₄haloalkyl;
R^{L4} is halo, —OR^{L5}, or C₁₋₄ alkyl optionally substituted by halogen, C₃₋₇ cycloalkyl, phenyl, 4- to 7-membered monocyclic saturated heterocycle, or 5- to 6-membered heteroaryl, wherein the C₃₋₇ cycloalkyl, phenyl, 4- to 7-membered monocyclic saturated heterocycle, and 5- to 6-membered heteroaryl are each optionally substituted with one to three substituents independently selected from halogen, C₁₋₄ alkyl, C₁₋₄ haloalkyl, C₁₋₄ alkoxy and C₁₋₄ haloalkoxy;
R^{L5} is H, C₁₋₄ alkyl or C₁₋₄ haloalkyl; 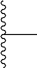
a bond to the IRAK binding moiety; and
-* represents a bond to the degradation signaling moiety DSM.

47. The compound of any one of claims 1 to 45, or a pharmaceutically acceptable salt thereof, wherein L is a bond, C₁₋₈ alkyl or is represented by formula (L-1), (L-2) or (L-3): 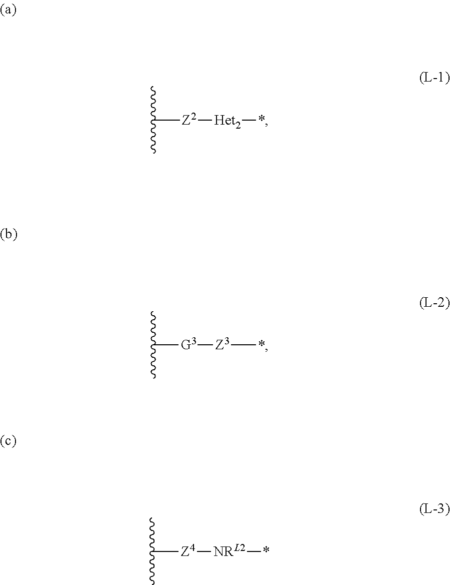
wherein:
Z² is a bond or C₁₋₄ alkyl optionally substituted with one or more halogen;
Het₂ is 4- to 7-membered heterocycle optionally substituted by one or more R^{L1};
G³ is C₃₋₇ cycloalkyl or 4- to 7-membered heterocycle; wherein the C₃₋₇ cycloalkyl and 4- to 7-membered heterocycle represented by G³ are each optionally substituted with one or more R^{L3};
Z³ is C₁₋₄ alkyl or *—C₁₋₄ alkyl-C(O)—*, wherein *- represents a bond connected to G3; -* is a bond connected to the DSM; and the C₁₋₄ alkyl is optionally substituted with one or more halogen;
Z⁴ is C₁₋₄ alkyl optionally substituted by R^{L4};
R^{L1} is, for each occurrence, independently selected from H, halogen, C₁₋₄ alkyl and C₁₋₄haloalkyl;
R^{L2} is H or C₁₋₄ alkyl;
R^{L3} is, for each occurrence, independently selected from H, halogen, C₁₋₄ alkyl and C₁₋₄haloalkyl;
R^{L4} is halo, —OR^{L5}, or C₁₋₄ alkyl optionally substituted by halogen, C₃₋₇ cycloalkyl, phenyl, 4- to 7-membered monocyclic saturated heterocycle, or 5- to 6-membered heteroaryl, wherein the C₃₋₇ cycloalkyl, phenyl, 4- to 7-membered monocyclic saturated heterocycle, and 5- to 6-membered heteroaryl are each optionally substituted with one to three substituents independently selected from halogen, C₁₋₄ alkyl, C₁₋₄ haloalkyl, C₁₋₄ alkoxy and C₁₋₄ haloalkoxy;
R^{L5} is H, C₁₋₄ alkyl or C₁₋₄ haloalkyl; 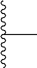
represents a bond to the IRAK binding moiety; and
-* represents a bond to the degradation signaling moiety DSM.

48. The compound of claim 46 or 47, or a pharmaceutically acceptable salt thereof, wherein:
Z² is a bond or —CH₂—;
Het₂ is selected from azetidinyl, piperidinyl and pyrrolidinyl; wherein the azetidinyl, piperidinyl and pyrrolidinyl represented by Het₂ are each optionally substituted by one or more R^{L1};
G³ is cyclohexyl or piperidinyl; wherein the cyclohexyl and piperidinyl represented by G³ are each optionally substituted with one or more R^{L3};
Z³ is —CH₂— or *—CH₂—C(O)—*; and
Z⁴ is —CH₂— optionally substituted by R^{L4}.

49. The compound of any one of claims 46 to 48, or a pharmaceutically acceptable salt thereof, wherein:
R^{L1} is H;
R^{L2} is H;
R^{L3} is H;
R^{L4} is benzyl.

50. The compound of any one of claims 46 to 48, or a pharmaceutically acceptable salt thereof, wherein L is represented by formula (L-1) and Het₂ is represented by one of the formulae: 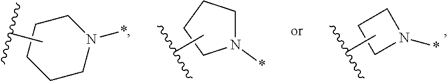
wherein: 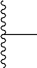
represents a bond to the IRAK binding moiety; and 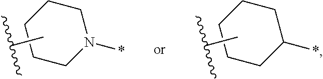
-* represents a bond to Z³.

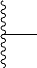

52. The compound of any one of claims 46 to 48, or a pharmaceutically acceptable salt thereof, wherein L is represented by formula (L-1) and Het₂ is: 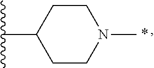 [SMILES: *C1CCN(C)CC1]
wherein: 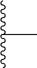
represents a bond to Z²; and
-* represents a bond to the degradation signaling moiety DSM.

53. The compound of any one of claims 46 to 48, or a pharmaceutically acceptable salt thereof, wherein L is represented by formula (L-2) and G³ is represented by: 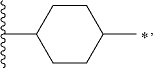 [SMILES: *C1CCC(C)CC1]
wherein: 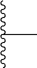
represents a bond to the IRAK binding moiety; and
-* represents a bond to Z³.

54. The compound of any one of claims 1 to 45, or a pharmaceutically acceptable salt thereof, wherein L is represented by any one of the following formulae: 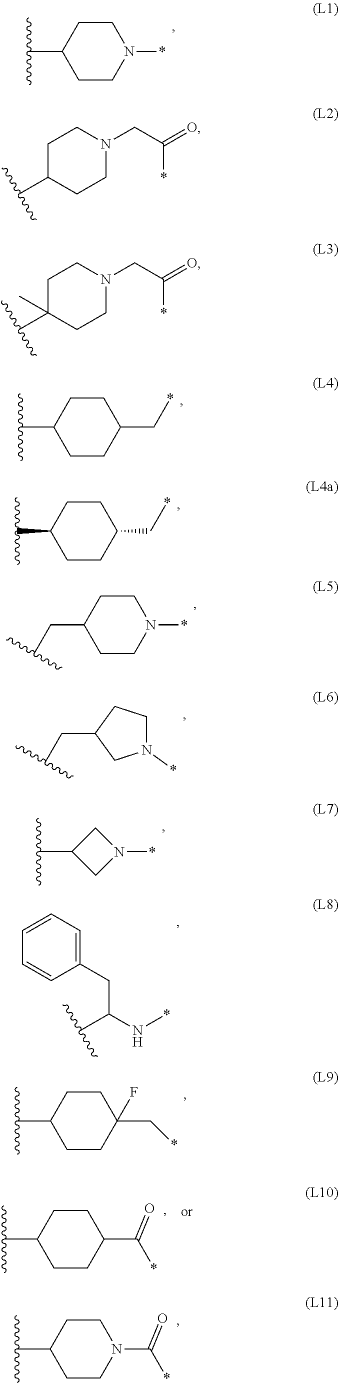
wherein: 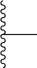
represents a bond to the IRAK binding moiety; and
-* represents a bond to the degradation signaling moiety DSM.

55. The compound of claim 1, wherein the compound is represented by the following formula: 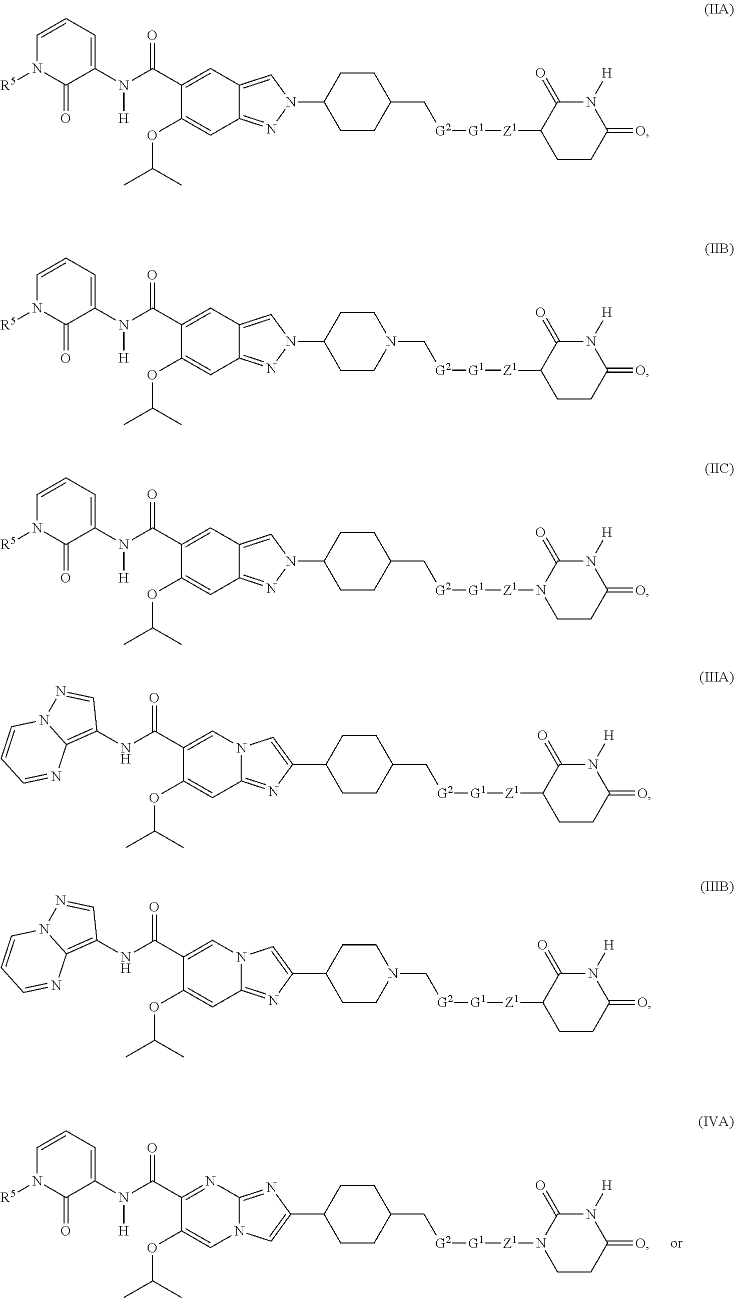 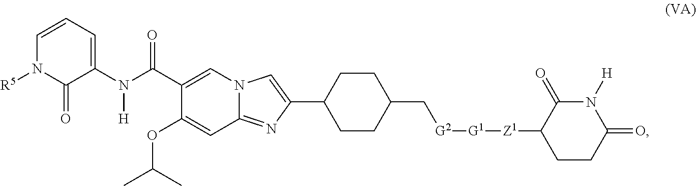
or a pharmaceutically acceptable salt thereof, wherein:
Z¹ is a bond or —O—;
G¹ is phenyl, 6-membered heteroaryl or 9-membered partially saturated bicyclic heterocycle, each of which is optionally substituted with 1 or 2 substituents independently selected from halo and C₁₋₂alkyl;
G² is Het₁, *—NR^{D4}-Het₁-*, or *—C(O)—C₁₋₂ alkyl-Het₁-*; wherein *- represents a bond to the linker L, and *- represents a bond to G¹;
Het₁ is piperidine optionally substituted with 1 or 2 halo or OH;
R⁵ is C₃₋₄cycloalkyl is optionally substituted with 1 halo; and
R^{D4} is H or C₁₋₂alkyl.

56. The compound of claim 55, or a pharmaceutically acceptable salt thereof, wherein:
G¹ is phenyl, pyridinyl, indazoyl, or 1,3-dihydro-2H-benzo[d]imidazol-2-one, each of which is optionally substituted with 1 or 2 substituents independently selected from halo and C₁₋₂alkyl;
G² is Het₁, *—NH-Het₁-*, or *—C(O)—CH₂—Het₁-*; wherein *- represents a bond to the linker L, and *- represents a bond to G¹;
Het₁ is piperidine optionally substituted with 1 or 2 halo or OH.

57. The compound of claim 56, or a pharmaceutically acceptable salt thereof, wherein:
G¹ is 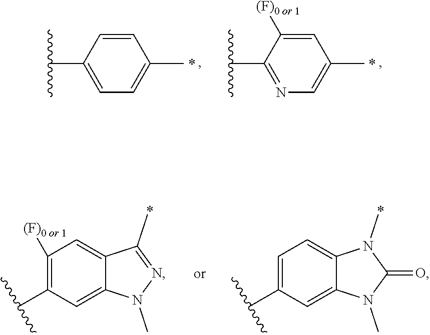
wherein 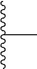
represents a bond to G², and -* represents a bond to Z¹;
Het₁ is 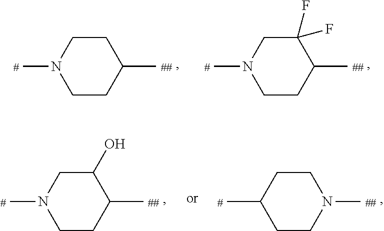
wherein #- represents a bond to the linker, —NH—, or —C(O)—CH₂— and ##- represents a bond to G¹;
R⁵ is cyclopropyl or

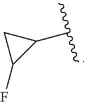 [SMILES: *C1CC1F]

58. The compound of claim 1, selected from a compound of any one of Examples 1 to 199 or a pharmaceutically acceptable salt thereof.

59. A pharmaceutical composition comprising a compound or a pharmaceutically acceptable salt thereof of any one of claims 1 to 58 and a pharmaceutically acceptable carrier.

60. A method of treating an IRAK4-mediated disease in a subject comprising administering to the subject a compound or a pharmaceutically acceptable salt thereof of any one of claims 1 to 58 or a pharmaceutical composition of claim 59.

61. The method of claim 60, wherein the IRAK4-mediated disease is selected from the group consisting of Rheumatoid Arthritis, Psoriatic arthritis, Osteoarthritis, Systemic Lupus Erythematosus, Lupus nephritis, Cutaneous Lupus Erythematosus, Ankylosing Spondylitis, Osteoporosis, Neuromyelitis optica, Systemic sclerosis, Psoriasis, Dermatomyositis, Atopic Dermatitis, Hidradenitis Suppurativa, Type I diabetes, Type II diabetes, Inflammatory Bowel Disease, Crohns's Disease, Ulcerative Colitis, Hyperimmunoglobulinemia D, periodic fever syndrome, Cryopyrin-associated periodic syndromes, Schnitzler's syndrome, Systemic juvenile idiopathic arthritis, Adult's onset Still's disease, Gout, Pseudogout, SAPHO syndrome, Castleman's disease, Sepsis, Stroke, Atherosclerosis, Celiac disease, Deficiency of IL-1 Receptor Antagonist, Alzheimer's disease, Parkinson's disease, Multiple Sclerosis and Cancer.

62. The method of claim 60, wherein the IRAK4-mediated disease is selected from the group consisting of an autoimmune disease, an inflammatory disease, bone diseases, metabolic diseases, neurological and neurodegenerative diseases and/or disorders, cardiovascular diseases, allergies, asthma, hormone-related diseases, Ischemic stroke, Cerebral Ischemia, hypoxia, Traumatic Brain Injury, Chronic Traumatic Encephalopathy, epilepsy, Parkinson's disease, and Amyotrophic Lateral Sclerosis.
